(19) United States
(12) Patent Application Publication
(10) Pub. No.: US 2025/0154154 A1
(43) Pub. Date: May 15, 2025

(54) SRC INHIBITORS AND USES THEREOF

(71) Applicants: Relay Binney Street, 2nd Floor, Cambridge, MA (US); D.E. Shaw Research, LLC, New York, NY (US)

(72) Inventors: Demetri T. MOUSTAKAS, Cambridge, MA (US); Lucian V. DIPIETRO, Cambridge, MA (US); Heike SCHOENHERR, Cambridge, MA (US); Bakary-Barry TOURÉ, Cambridge, MA (US); W. Patrick WALTERS, Cambridge, MA (US); Mark A. MURCKO, Cambridge, MA (US); Ravi KURUKULASURIYA, Cambridge, MA (US); Levi Charles Thomas PIERCE, Cambridge, MA (US); Fabrizio GIORDANETTO, New York, NY (US); Yibing SHAN, New York, NY (US); Shiori SAGAWA, New York, NY (US); Goran KRILOV, New York, NY (US); Fiona MCROBB, New York, NY (US); Eric THERRIEN, New York, NY (US)

(21) Appl. No.: 18/037,253

(22) PCT Filed: Nov. 17, 2021
(86) PCT No.: PCT/US2021/072449

§ 371 Date: May 16, 2023

Related U.S. Application Data
(60) Provisional application No. 63/114,765, filed on Nov. 17, 2020.

(51) Int. Cl. C07D 487/04; A61K 31/519; A61K 31/53; A61K 31/5377; A61K 31/538; A61K 31/55

(52) U.S. Cl. CPC C07D 487/04; A61K 31/519; A61K 31/53; A61K 31/5377; A61K 31/538; A61K 31/55

(57) ABSTRACT

The present invention relates to compounds and methods useful for inhibiting non-receptor tyrosine-protein kinase Src (“Src”). The invention also provides pharmaceutically acceptable compositions comprising compounds of the present invention and methods of using said compositions in the treatment of various disorders.

### CROSS-REFERENCE TO RELATED APPLICATIONS

This application claims the benefit of U.S. Provisional Application No. 63/114,765, filed on Nov. 17, 2020, the entirety of which is hereby incorporated by reference.

### TECHNICAL FIELD OF THE INVENTION

The present invention relates to compounds and methods useful for inhibiting non-receptor tyrosine-protein kinase Src (“Src”). The invention also provides pharmaceutically acceptable compositions comprising compounds of the present invention and methods of using said compositions in the treatment of various disorders.

### BACKGROUND OF THE INVENTION

Cancer is a disease that begins with mutation of critical genes: oncogenes and tumor suppressor genes. Mutation of critical genes allows a cancer cell to evolve, and ultimately results in pathogenic replication (a loss of normal regulatory control leading to excessive cell proliferation) of various types of cells in the human body. Conventional cancer treatments have focused mainly on killing cancerous cells. Such treatments threaten noncancerous cells too, are inherently stressful to the human body, produce many side effects, and are of uncertain efficacy. More importantly, such treatment regimens may not be directed toward the actual root of the cancer or its prevention.

Other diseases are also associated with excessive cell death. For example, diseases associated with the loss of neurons in different regions of the central nervous system (CNS), including, for example, brain tissue and the spinal cord, such as Alzheimer's disease, amyotrophic lateral sclerosis (“ALS” or “Lou Gehrig's disease”), Parkinson's disease, Huntington's disease, brain aging, Friedreich's ataxia, multiple sclerosis, diabetic necrosis, ischaemia, and stroke. These types of diseases are exemplary of diseases and disorders collectively referred to as “neurodegenerative diseases.” Treatment and prevention of neurodegenerative disorders remains elusive in that many proposed treatment methods are not practical since exogenous administration of numerous putative therapeutics is not efficacious due to their general inability to cross the blood-brain barrier.

Inflammation is a process by which the body's white blood cells react to infection by foreign substances, such as bacteria and viruses. It is usually characterized by pain, swelling, warmth and redness of the affected tissue. Effector substances known as cytokines and prostaglandins control this process, and are released in an ordered and self-limiting cascade into the blood or affected tissues. The release of such effector substances increases the blood flow to the area of injury or infection. Some of the effector substances cause a leak of fluid into the tissues, resulting in swelling. This protective process may stimulate nerves and cause pain. These changes, when occurring for a limited period in the relevant area, work to the benefit of the body.

Thus, there is a need in the art for therapeutic methods treating or preventing cancer, a neurodegenerative disease, or an inflammatory disease.

### SUMMARY OF THE INVENTION

In certain embodiments, the invention relates to a compound of Formula (I) or (II):

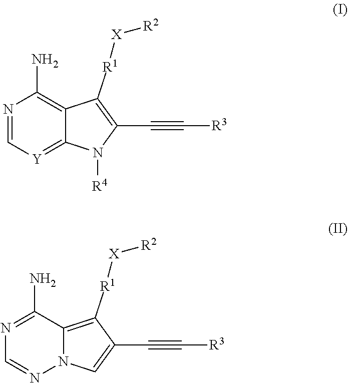

or a pharmaceutically acceptable salt thereof, wherein each variable is as defined and described in embodiments herein, both singly and in combination.

In certain embodiments, the present invention provides a compound of Formula (III) or (IV):

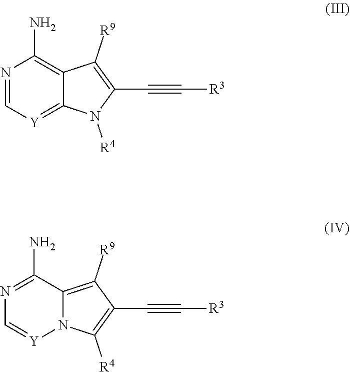

or a pharmaceutically acceptable salt thereof, wherein each variable is as defined and described in embodiments herein, both singly and in combination.

In certain embodiments, the present invention provides a compound of Formula (V) or (VI):

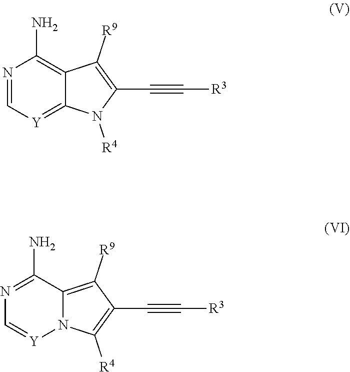

or a pharmaceutically acceptable salt thereof, wherein each variable is as defined and described in embodiments herein, both singly and in combination.

In certain embodiments, the invention relates to a method for treating or preventing cancer, a neurodegenerative disease, or an inflammatory disease, comprising the step of: administering to a subject in need thereof a therapeutically effective amount of any one of the compounds described herein.

### DETAILED DESCRIPTION OF THE INVENTION

In certain embodiments, the invention relates to compounds useful for the treatment of cancer, neurodegenerative diseases, or inflammatory diseases.

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/of” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl groups may be substituted or unsubstituted.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Likewise, preferred cycloalkyls have from 3-10 carbon atoms in their ring structure, and more preferably have 3-6 carbons in the ring structure. Cycloalkyl groups may be substituted or unsubstituted.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “alkylthio” refers to an alkyl group, as defined above, having a sulfur moiety attached thereto. In certain embodiments, the “alkylthio” moiety is represented by one of —(S)-alkyl, —(S)-alkenyl, —(S)-alkynyl, and —(S)—(CH₂)ₘ—R¹, wherein m and R¹ are defined below. Representative alkylthio groups include methylthio, ethylthio, and the like. The terms “alkoxyl” or “alkoxy” as used herein refers to an alkyl group, as defined below, having an oxygen moiety attached thereto. Representative alkoxyl groups include methoxy, ethoxy, propoxy, tert-butoxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₁₀, where m and R₁₀ are described below.

The terms “amine” and “amino” are art-recognized and refer to both unsubstituted and substituted amines, e.g., a moiety that can be represented by the formulae:

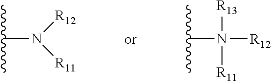

wherein R₁₁, R₁₂ and R₁₃ each independently represent a hydrogen, an alkyl, an alkenyl, —(CH₂)ₘ—R₁₀, or R₁₁ and R₁₂ taken together with the N atom to which they are attached complete a heterocycle having from 4 to 8 atoms in the ring structure; R₁₀ represents an alkenyl, aryl, cycloalkyl, a cycloalkenyl, a heterocyclyl, or a polycyclyl; and m is zero or an integer in the range of 1 to 8. In certain embodiments, only one of R₁₁ or R₁₂ can be a carbonyl, e.g., R₁₁, R₁₂, and the nitrogen together do not form an imide. In even more certain embodiments, R₁₁ and R₁₂ (and optionally R₁₃) each independently represent a hydrogen, an alkyl, an alkenyl, or —(CH₂)ₘ—R₁₀. Thus, the term “alkylamine” as used herein means an amine group, as defined above, having a substituted or unsubstituted alkyl attached thereto, i.e., at least one of R₁₁ and R₁₂ is an alkyl group. In certain embodiments, an amino group or an alkylamine is basic, meaning it has a conjugate acid with a pKₐ>7.00, i.e., the protonated forms of these functional groups have pKₐs relative to water above about 7.00.

The term “amide”, as used herein, refers to a group

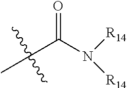

wherein each R₁₄ independently represent a hydrogen or hydrocarbyl group, or two R₁₄ are taken together with the N atom to which they are attached complete a heterocycle having from 4 to 8 atoms in the ring structure.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

Unless otherwise defined, the terms “heterocyclyl” or “heterocyclic group” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. Unless otherwise defined, the heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like. The terms “heterocycle” and “heterocyclic ring” refer to the same structures as “heterocyclyl” and “heterocyclic group”.

The term “carbonyl” is art-recognized and includes such moieties as can be represented by the formula:

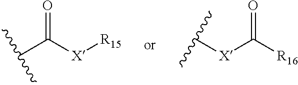

wherein X′ is a bond or represents an oxygen or a sulfur, and R₁₅ represents a hydrogen, an alkyl, an alkenyl, —(CH₂)ₘ—R₁₀ or a pharmaceutically acceptable salt, R₁₆ represents a hydrogen, an alkyl, an alkenyl or —(CH₂)ₘ—R₁₀, where m and R₁₀ are as defined above. Where X′ is an oxygen and R₁₅ or R₁₆ is not hydrogen, the formula represents an “ester.” Where X′ is an oxygen, and R₁₅ is as defined above, the moiety is referred to herein as a carboxyl group, and particularly when R₁₅ is a hydrogen, the formula represents a “carboxylic acid”. Where X′ is an oxygen, and R₁₆ is a hydrogen, the formula represents a “formate.” In general, where the oxygen atom of the above formula is replaced by a sulfur, the formula represents a “thiocarbonyl” group. Where X′ is a sulfur and R₁₅ or R₁₆ is not hydrogen, the formula represents a “thioester” group. Where X′ is a sulfur and R₁₅ is a hydrogen, the formula represents a “thiocarboxylic acid” group. Where X′ is a sulfur and R₁₆ is a hydrogen, the formula represents a “thioformate” group. On the other hand, where X′ is a bond, and R₁₅ is not hydrogen, the above formula represents a “ketone” group. Where X′ is a bond, and R₁₅ is a hydrogen, the above formula represents an “aldehyde” group.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “nitro” means —NO₂; the term “halogen” designates —F, —Cl, —Br, or —I; the term “sulfhydryl” means —SH; the term “hydroxyl” means —OH; the term “sulfonyl” means —SO₂—; the term “azido” means —N₃; the term “cyano” means —CN; the term “isocyanato” means —NCO; the term “thiocyanato” means —SCN; the term “isothiocyanato” means —NCS; and the term “cyanato” means —OCN.

The term “sulfamoyl” is art-recognized and includes a moiety that can be represented by the formula:

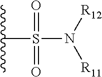

in which R₁₁ and R₁₂ are as defined above.

The term “sulfate” is art recognized and includes a moiety that can be represented by the formula:

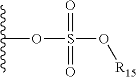

in which R₁₅ is as defined above.

The term “sulfonamide” is art recognized and includes a moiety that can be represented by the formula:

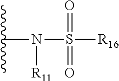

in which R₁₁ and R₁₆ are as defined above.

The term “sulfonate” is art-recognized and includes a moiety that can be represented by the formula:

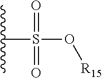

in which R₁₅ is as defined above.

The terms “sulfoxido” or “sulfinyl”, as used herein, refers to a moiety that can be represented by the formula:

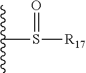

in which R₁₇ is selected from the group consisting of the group consisting of hydrogen, alkyl, alkenyl, alkynyl, cycloalkyl, heterocyclyl, aralkyl, or aryl.

The term “urea” is art-recognized and may be represented by the general formula

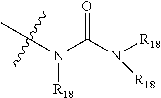

wherein each R₁₈ independently represents hydrogen or a hydrocarbyl, such as alkyl, or any occurrence of R₁₈ taken together with another and the intervening atom(s) complete a heterocycle having from 4 to 8 atoms in the ring structure.

As used herein, the definition of each expression, e.g., alkyl, m, n, etc., when it occurs more than once in any structure, is intended to be independent of its definition elsewhere in the same structure.

For purposes of this invention, the chemical elements are identified in accordance with the Periodic Table of the Elements, CAS version, Handbook of Chemistry and Physics, 67th Ed., 1986-87, inside cover.

Compounds of the present invention include those described generally herein, and are further illustrated by the classes, subclasses, and species disclosed herein. As used herein, the following definitions shall apply unless otherwise indicated. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

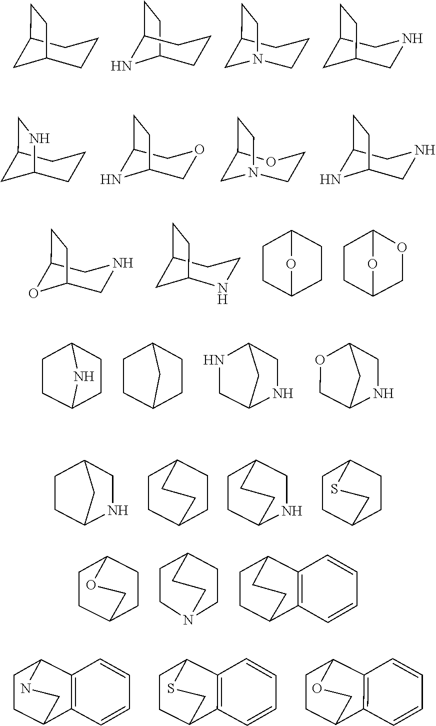

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, 9, or 10 ring atoms, more preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As used herein, unless otherwise defined, the terms “monocycle,” “monocyclic” and the like refer to a ring that shares no ring atoms with any other ring. Monocycles may be aromatic, partially unsaturated, or saturated, and may be carbocyclic or heterocyclic.

As used herein, unless otherwise defined, the terms “polycycle,” “polycyclic” and the like refer to a ring system comprising two or more rings wherein each of the component rings shares at least one ring atom with at least one other ring in the ring system. Each component ring of a polycycle may be aromatic, partially unsaturated, or saturated, and may be carbocyclic or heterocyclic.

As used herein, unless otherwise defined, the terms “bicycle,” “bicyclic” and the like refer to a polycycle ring system having two rings, wherein the two rings share at least one ring atom with each other. Each component ring of a bicycle may be aromatic, partially unsaturated, or saturated, and may be carbocyclic or heterocyclic.

As used herein, the terms “spirocycle,” “spirocyclic,” “spiro-” and the like refer to a polycyclic ring system, wherein two rings of the ring system share only one ring atom. Spirocycles may contain additional rings, including other spiro-fused rings.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)(OR^{∘})R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, =NNHC(O)R*, =NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a warhead moiety, R¹, of a provided compound comprises one or more deuterium atoms. In certain embodiments, Ring B of a provided compound may be substituted with one or more deuterium atoms.

As used herein, the term “inhibitor” is defined as a compound that binds to and/or inhibits Src with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P ³³P ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in a Src protein kinase activity between a sample comprising a compound of the present invention, or composition thereof, and a Src protein kinase, and an equivalent sample comprising an Src protein kinase, in the absence of said compound, or composition thereof.

### EXEMPLARY COMPOUNDS OF THE INVENTION

In certain embodiments, the invention relates to a compound of Formula (I) or (II):

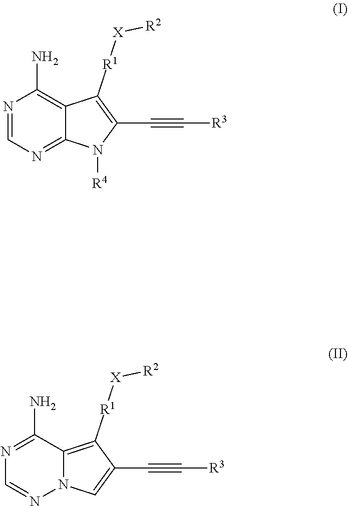

wherein:
R¹ is selected from optionally substituted arylene and heteroarylene;
R² is selected from optionally substituted aryl, heteroaryl, and cycloalkyl;
R³ is selected from

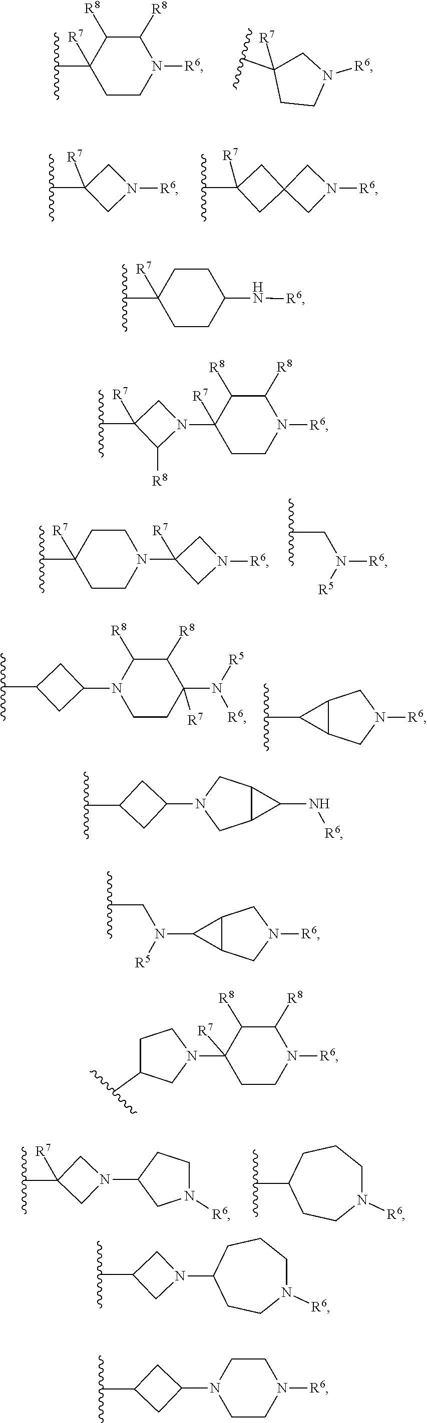

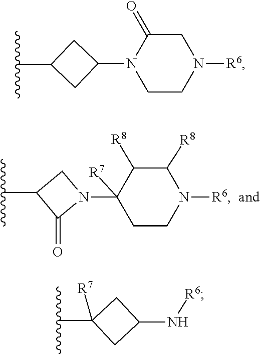

R⁴ is selected from H, alkyl, alkylene-OH, alkylene-O-alkyl, heterocycloalkyl, cycloalkyl-OH, alkylene-C(O)—NH₂, alkylene-C(O)—OR₅, alkylene-heterocycloalkyl, and alkylene-heteroaryl;
R⁵ is alkyl or H;
R⁶ is CN, —C(O)C(H)CH₂, —C(O)CH₂Cl, —C(O)C(H)(Cl)CH₃, —S(O)₂C(H)CH₂, —S(O)₂C(H)CH₂—CH₃, —C(O)CCMe, —C(O)-alkenylene-N(alkyl)₂, —C(O)-alkenylene-NH(alkyl), —C(O)-alkenylene-OMe, —C(O)-alkenylene-(N-heterocycloalkyl), —C(O)— alkylene-N(H)C(O)C(H)CH₂, —C(O)-alkylene-N(Me)C(O)C(H)CH₂, —C(O)C(CH₃)CH₂, or —C(O)C(H)C(H)CH₃;
R⁷ is independently selected from H, OH, Me, F, and OMe;
R⁸ is independently selected from H, OH, Me, F, CH₂OH, CH₂OMe and OMe; and
X is —O—, —N(H)—, or —C(H)(CN)—;
   or a pharmaceutically acceptable salt thereof.

In some embodiments, the compound is a compound of Formula (I), or a pharmaceutically acceptable salt thereof. In some embodiments, the compound is a compound of Formula (II), or a pharmaceutically acceptable salt thereof.

In certain embodiments, the present invention provides a compound of Formula (III) or (IV), or a pharmaceutically acceptable salt thereof:

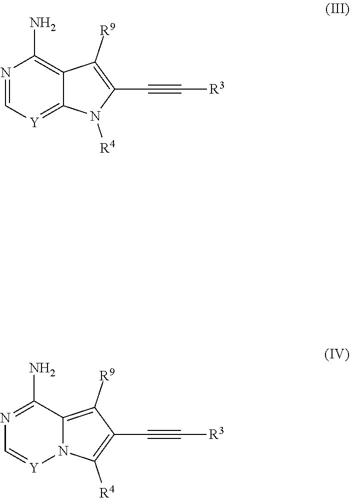

wherein:
Y is CR^{Y} or N;
R³ is —Z¹-L¹-Z²-L²-R⁶;
Z¹ is C₁₋₆ aliphatic; a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z¹ is substituted by m instances of R⁷, and n instances of R⁸;
Z² is a covalent bond; C₁₋₆ aliphatic; a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z² is substituted by m instances of R⁷, and n instances of R⁸;
each of L¹ and L² is independently a covalent bond, —N(H)—, —N(R⁵)—, or —C(O)—;
R⁴ is hydrogen, or an optionally substituted group selected from the group consisting of C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R⁶ is —CN,

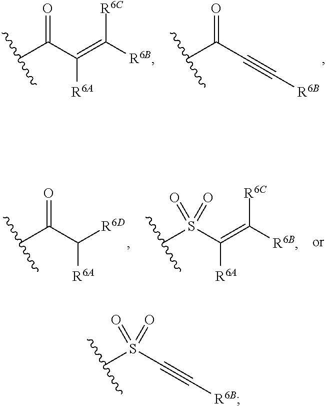

each of R^{6A}, R^{6B}, and R^{6C} is independently halogen, —CN, —C(O)R, —C(O)OR, —C(O)NR₂, —C(O)N(R)OR, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R^{6D} is halogen or —OS(O)₂R;
each instance of R⁵, R⁷, R⁸, and R¹⁰ is independently hydrogen or R^{L};
each instance of R^{L} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R^{Y} is hydrogen or R^{L};
R⁹ is —R¹—X—R²; or a ring selected from the group consisting of phenyl; a 5-6 membered monocyclic heteroaryl ring having 1-4 heteroatoms independently selected from nitrogen, oxygen, and sulfur; and an 8-10 membered bicyclic ring having 1-4 heteroatoms independently selected from nitrogen, oxygen, and sulfur; wherein said ring is substituted by p instances of R¹⁰;
R¹ is selected from arylene and heteroarylene; each of which is optionally substituted;
R² is selected from aryl, heteroaryl, and cycloalkyl; each of which is optionally substituted;
X is a C₁₋₂ bivalent hydrocarbon chain wherein one or more methylene units of the chain are optionally and independently replaced by —CH(R^{L})—, C₃₋₅ cycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each instance of m and n is independently 0-4; and
p is 0-4.

In some embodiments, the compound is a compound of Formula (III), or a pharmaceutically acceptable salt thereof. In some embodiments, the compound is a compound of Formula (IV), or a pharmaceutically acceptable salt thereof.

In certain embodiments, the present invention provides a compound of Formula (V) or (VI), or a pharmaceutically acceptable salt thereof:

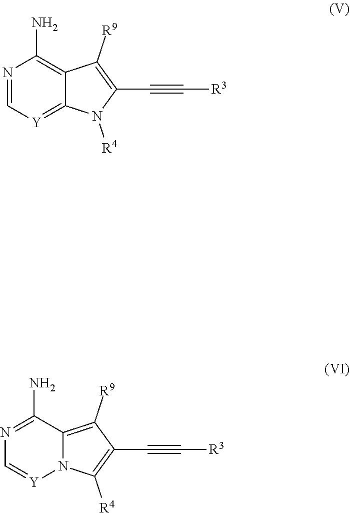

wherein:
Y is CR^{Y} or N;
R³ is —Z¹-L¹-Z²-L²-R⁶;
Z¹ is C₁₋₆ aliphatic; a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z¹ is substituted by m instances of R⁷, and n instances of R⁸;
Z² is a covalent bond; C₁₋₆ aliphatic; a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z² is substituted by m instances of R⁷, and n instances of R⁸;
each of L¹ and L² is independently a covalent bond, —N(H)—, —N(R⁵)—, or —C(O)—;
R⁴ is hydrogen, or an optionally substituted group selected from the group consisting of C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R⁶ is —CN,

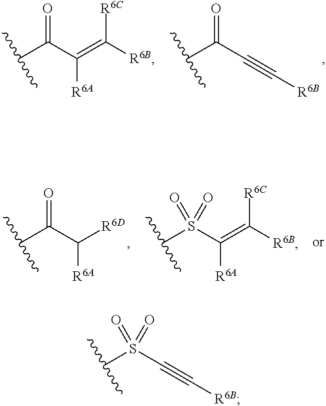

each of R^{6A}, R^{6B}, and R^{6C} is independently hydrogen, halogen, —CN, —C(O)R, —C(O)OR, —C(O)NR₂, —C(O)N(R)OR, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R^{6D} is halogen or —OS(O)₂R;
each instance of R⁵, R⁷, R⁸, and R¹⁰ is independently hydrogen or R^{L};
each instance of R^{L} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R^{Y} is hydrogen or R^{L};
R⁹ is —R¹—X—R²; or a ring selected from the group consisting of phenyl; a 5-6 membered monocyclic heteroaryl ring having 1-4 heteroatoms independently selected from nitrogen, oxygen, and sulfur; and an 8-10 membered bicyclic ring having 1-4 heteroatoms independently selected from nitrogen, oxygen, and sulfur; wherein said ring is substituted by p instances of R¹⁰;
R¹ is selected from arylene and heteroarylene; each of which is optionally substituted;
R² is selected from aryl, heteroaryl, and cycloalkyl; each of which is optionally substituted;
X is a C₁₋₂ bivalent hydrocarbon chain wherein one or more methylene units of the chain are optionally and independently replaced by —CH(R^{L})—, C₃₋₅ cycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each instance of m and n is independently 0-4; and
p is 0-4.

As defined generally above, Y is CR^{Y} or N.

In some embodiments, Y is CR^{Y}. In some embodiments, Y is N.

In some embodiments, Y is selected from those depicted in the compound tables below.

As defined generally above, R³ is —Z¹-L¹-Z²-L²-R⁶.

In some embodiments, R³ is

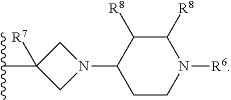

In some embodiments, R³ is

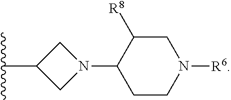

In some embodiments, R³ is

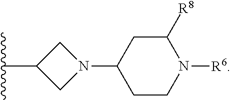

In some embodiments, R³ is

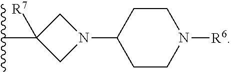

In some embodiments, R³ is

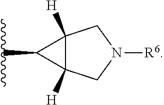

In some embodiments, R³ is

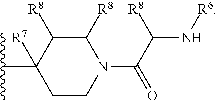

In some embodiments, R³ is

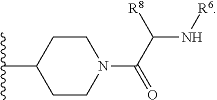

In some embodiments, R³ is:

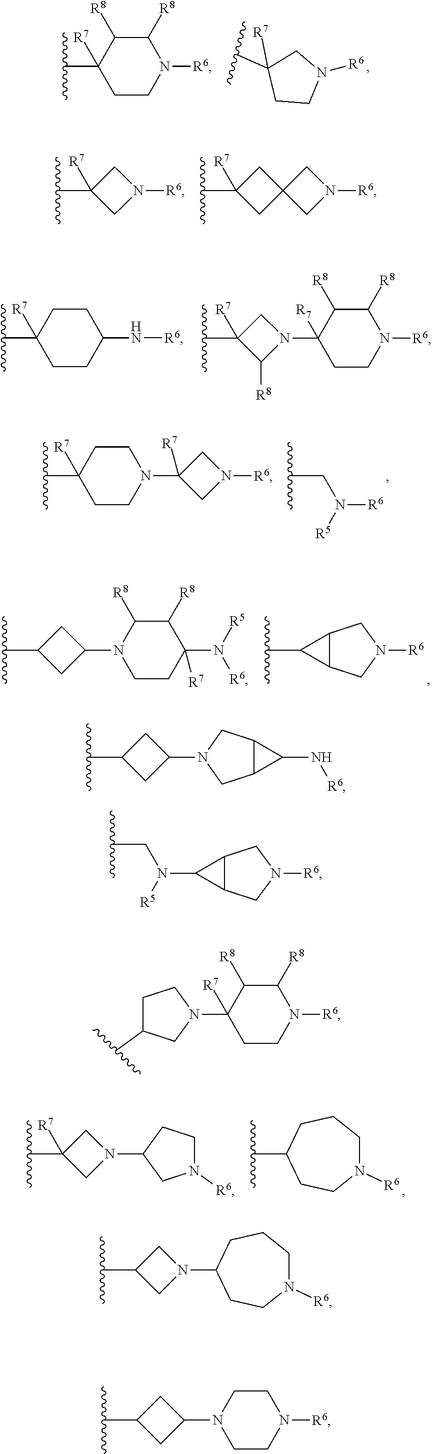

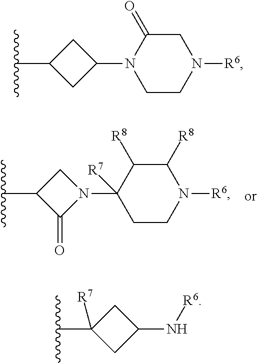

In some embodiments, R³ is

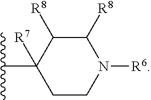

In some embodiments, R³ is R³ is

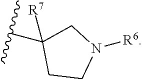

In some embodiments, R³ is

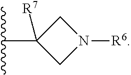

In some embodiments, R³ is

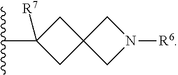

In some embodiments, R³ is

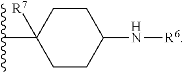

In some embodiments, R³ is

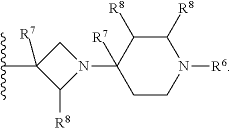

In some embodiments, R³ is

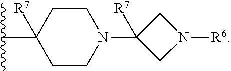

In some embodiments, R³ is

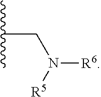

In some embodiments, R³ is

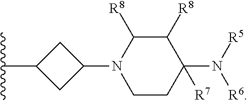

In some embodiments, R³ is

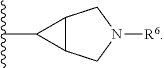

In some embodiments, R³ is

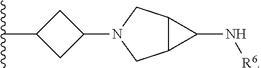

In some embodiments, R³ is

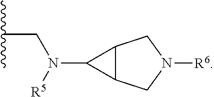

In some embodiments, R³ is

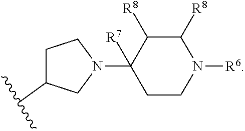

In some embodiments, R³ is

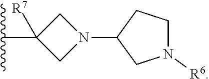

In some embodiments, R³ is

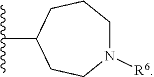

In some embodiments, R³ is

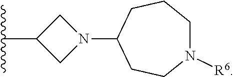

In some embodiments, R³ is

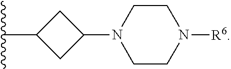

In some embodiments, R³ is

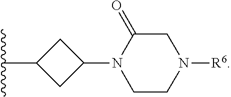

In some embodiments, R³ is

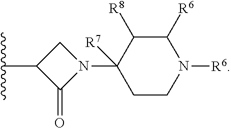

In some embodiments, R³ is

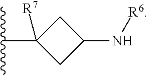

In some embodiments, R³ is

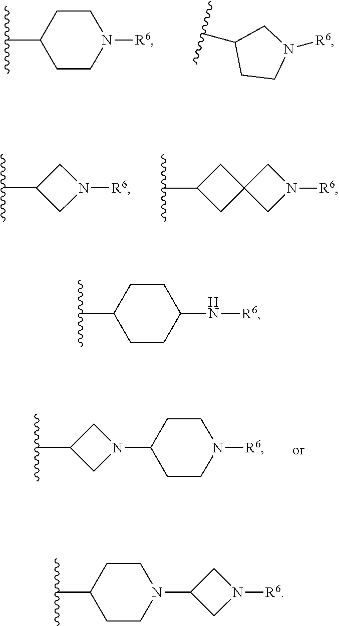

In some embodiments R³ is

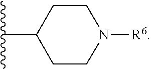

In some embodiments, R³ is

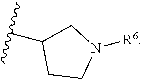

In some embodiments, R³ is

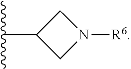

In some embodiments, R³ is

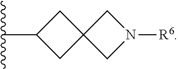

In some embodiments, R³ is

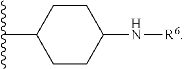

In some embodiments, R³ is

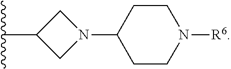

In some embodiments, R³ is

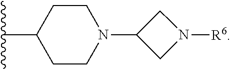

In some embodiments, R³ is selected from those depicted in the compound tables below.

As defined generally above, Z¹ is C₁₋₆ aliphatic; a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z¹ is substituted by m instances of R⁷, and n instances of R⁸.

In some embodiments, Z¹ is C₁₋₆ aliphatic; wherein Z¹ is substituted by m instances of R⁷, and n instances of R⁸. In some embodiments, Z¹ is a 3-7 membered monocyclic heterocyclic ring having 1-2 heteroatoms independently selected from nitrogen, oxygen, and sulfur; wherein Z¹ is substituted by m instances of R⁷, and n instances of R⁸. In some embodiments, Z¹ is a 6-10 membered bicyclic heterocyclic ring having 1-2 heteroatoms independently selected from nitrogen, oxygen, and sulfur; wherein Z¹ is substituted by m instances of R⁷, and n instances of R⁸.

In some embodiments, Z¹ is C₃₋₆ cycloaliphatic; a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z¹ is substituted by m instances of R⁷, and n instances of R⁸.

In some embodiments, Z¹ is C₃₋₆ cycloaliphatic or a 3-7 membered monocyclic heterocyclic ring having 1-2 heteroatoms independently selected from nitrogen, oxygen, and sulfur; wherein Z¹ is substituted by m instances of R⁷, and n instances of R⁸.

In some embodiments, Z¹ is a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z¹ is substituted by m instances of R⁷, and n instances of R⁸.

In some embodiments, Z¹ is selected from those depicted in the compound tables below.

As defined generally above, Z² is a covalent bond; C₁₋₆ aliphatic; a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z² is substituted by m instances of R⁷, and n instances of R⁸.

In some embodiments, Z² is a covalent bond. In some embodiments, Z² is C₁₋₆ aliphatic; a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z² is substituted by m instances of R⁷, and n instances of R⁸.

In some embodiments, Z² is C₁₋₆ aliphatic; wherein Z² is substituted by m instances of R⁷, and n instances of R⁸. In some embodiments, Z² is a 3-7 membered monocyclic heterocyclic ring having 1-2 heteroatoms independently selected from nitrogen, oxygen, and sulfur; wherein Z² is substituted by m instances of R⁷, and n instances of R⁸. In some embodiments, Z² is a 6-10 membered bicyclic heterocyclic ring having 1-2 heteroatoms independently selected from nitrogen, oxygen, and sulfur; wherein Z² is substituted by m instances of R⁷, and n instances of R⁸.

In some embodiments, Z² is C₃₋₆ cycloaliphatic; a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z² is substituted by m instances of R⁷, and n instances of R⁸. In some embodiments, Z² is a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z² is substituted by m instances of R⁷, and n instances of R⁸.

In some embodiments, at least one of Z¹ and Z² is a cyclic group.

In some embodiments, Z² is selected from those depicted in the compound tables below.

As defined generally above, L¹ is a covalent bond, —N(H)—, —N(R⁵)—, or —C(O)—.

In some embodiments, L¹ is a covalent bond. In some embodiments, L¹ is —N(H)—, —N(R⁵)—, or —C(O)—. In some embodiments, L¹ is —N(H)—. In some embodiments, L¹ is —N(R⁵)—. In some embodiments, L¹ is —C(O)—.

In some embodiments, L¹ is selected from those depicted in the compound tables below.

As defined generally above, L² is a covalent bond, —N(H)—, —N(R⁵)—, or —C(O)—.

In some embodiments, L² is a covalent bond. In some embodiments, L² is —N(H)—, —N(R⁵)—, or —C(O)—. In some embodiments, L² is —N(H)—. In some embodiments, L² is —N(R⁵)—. In some embodiments, L² is —C(O)—.

In some embodiments, L² is selected from those depicted in the compound tables below.

As defined generally above, R⁴ is hydrogen, or an optionally substituted group selected from the group consisting of C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁴ is hydrogen. In some embodiments, R⁴ is an optionally substituted group selected from the group consisting of C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁴ is an optionally substituted C₁₋₆ aliphatic. In some embodiments, R⁴ is an optionally substituted phenyl. In some embodiments, R⁴ is an optionally substituted 3-7 membered saturated or partially unsaturated heterocyclic ring having 1-2 heteroatoms independently selected from nitrogen, oxygen, and sulfur. In some embodiments, R⁴ is an optionally substituted 5-6 membered heteroaryl ring having 1-4 heteroatoms independently selected from nitrogen, oxygen, and sulfur.

In some embodiments, R⁴ is C₁₋₆ aliphatic. In some embodiments, R⁴ is phenyl. In some embodiments, R⁴ is a 3-7 membered saturated or partially unsaturated heterocyclic ring having 1-2 heteroatoms independently selected from nitrogen, oxygen, and sulfur. In some embodiments, R⁴ is a 5-6 membered heteroaryl ring having 1-4 heteroatoms independently selected from nitrogen, oxygen, and sulfur. In some embodiments, R⁴ is C₁₋₃ aliphatic. In some embodiments, R⁴ is i-propyl or methyl. In some embodiments, R⁴ is methyl.

In some embodiments, R⁴ is an optionally substituted C₁₋₆ aliphatic or phenyl. In some embodiments, R⁴ is an optionally substituted 3-7 membered saturated or partially unsaturated heterocyclic ring having 1-2 heteroatoms independently selected from nitrogen, oxygen, and sulfur, or an optionally substituted 5-6 membered heteroaryl ring having 1-4 heteroatoms independently selected from nitrogen, oxygen, and sulfur.

In some embodiments, R⁴ is an optionally substituted C₁₋₆ aliphatic or 3-7 membered saturated or partially unsaturated heterocyclic ring having 1-2 heteroatoms independently selected from nitrogen, oxygen, and sulfur. In some embodiments, R⁴ is an optionally substituted phenyl or 5-6 membered heteroaryl ring having 1-4 heteroatoms independently selected from nitrogen, oxygen, and sulfur.

In some embodiments, R⁴ is hydrogen, or an optionally substituted C₁₋₆ aliphatic or 3-7 membered saturated or partially unsaturated heterocyclic ring having 1-2 heteroatoms independently selected from nitrogen, oxygen, and sulfur. In some embodiments, R⁴ is an optionally substituted C₁₋₆ aliphatic or 5-6 membered saturated heterocyclic ring having 1-2 heteroatoms independently selected from nitrogen and oxygen.

In some embodiments, R⁴ is hydrogen or C₁₋₆ aliphatic optionally substituted with —OH, —O—(C₁₋₆ aliphatic), —C(O)NH₂, —C(O)OH, or —C(O)—O—(C₁₋₆ aliphatic). In some embodiments, R⁴ is C₁₋₆ aliphatic optionally substituted with —OH, —O—(C₁₋₆ aliphatic), —C(O)NH₂, —C(O)OH, —C(O)—O—(C₁₋₆ aliphatic).

In some embodiments, R⁴ is C₁₋₆ aliphatic substituted with —OH. In some embodiments, R⁴ is C₁₋₆ aliphatic substituted with —O—(C₁₋₆ aliphatic). In some embodiments, R⁴ is optionally substituted C₃₋₆ cycloalkyl. In some embodiments, R⁴ is C₃₋₆ cycloalkyl substituted with —OH. In some embodiments, R⁴ is C₃₋₆ cycloalkyl. In some embodiments, R⁴ is C₁₋₆ aliphatic substituted with —C(O)NH₂. In some embodiments, R⁴ is C₁₋₆ aliphatic substituted with —C(O)OH. In some embodiments, R⁴ is C₁₋₆ aliphatic substituted with —C(O)—O—(C₁₋₆ aliphatic).

In some embodiments, R⁴ is selected from those depicted in the compound tables below.

As defined generally above, R⁶ is —CN,

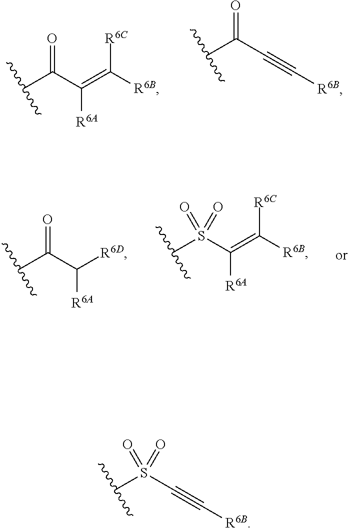

In some embodiments, R⁶ is —CN. In some embodiments, R⁶ is

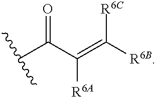

In some embodiments, R⁶ is

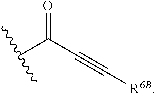

In some embodiments, R⁶ is

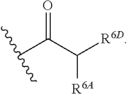

In some embodiments, R⁶ is

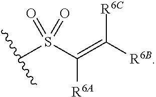

In some embodiments, R⁶ is

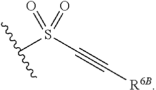

In some embodiments, R⁶ is —CN,

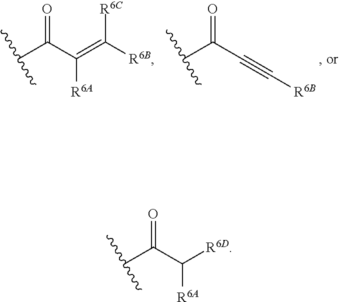

In some embodiments, R⁶ is

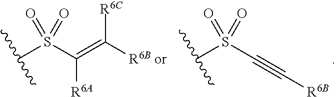

In some embodiments, R⁶ is

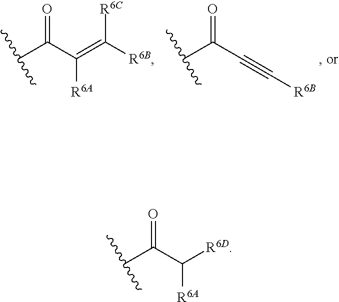

In some embodiments, R⁶ is

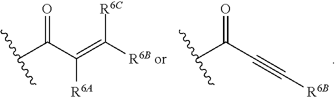

In some embodiments, R⁶ is

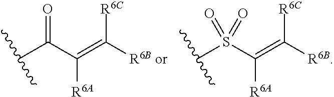

In some embodiments, R⁶ is

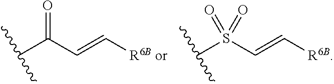

In some embodiments, R⁶ is

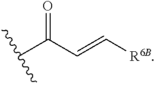

In some embodiments, R⁶ is

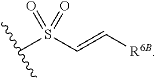

In some embodiments, R⁶ is CN, —C(O)C(H)CH₂, —C(O)CH₂Cl, —C(O)C(H)(Cl)CH₃, —S(O)₂C(H)CH₂, —S(O)₂C(H)CH—CH₃, —C(O)CCMe, —C(O)C(CH₃)CH₂, or —C(O)C(H)C(H)CH₃.

In some embodiments, R⁶ is —CN. In some embodiments, R⁶ is —C(O)C(H)CH₂. In some embodiments, R⁶ is —C(O)CH₂Cl. In some embodiments, R⁶ is —C(O)C(H)(Cl)CH₃. In some embodiments, R⁶ is —S(O)₂C(H)CH₂. In some embodiments, R⁶ is S(O)₂C(H)CH₂—CH₃. In some embodiments, R⁶ is —C(O)CCMe. In some embodiments, R⁶ is —C(O)C(CH₃)CH₂. In some embodiments, R⁶ is —C(O)C(H)C(H)CH₃.

In some embodiments, R⁶ is selected from those depicted in the compound tables below.

In some embodiments, R⁶ is -L-Y^{W}, wherein:
L is a covalent bond or a bivalent C₁₋₈ saturated or unsaturated, straight or branched, hydrocarbon chain, wherein one, two, or three methylene units of L are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—;
Y^{W} is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 R^{e} groups; and
each R^{e} is independently selected from -Q-Z, oxo, NO₂, halogen, CN, a suitable leaving group, or a C₁₋₆ aliphatic optionally substituted with oxo, halogen, NO₂, or CN, wherein:
Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and
Z is hydrogen or C₁₋₆ aliphatic optionally substituted with oxo, halogen, NO₂, or CN.

In certain embodiments, L is a covalent bond.

In certain embodiments, L is a bivalent C₁₋₈ saturated or unsaturated, straight or branched, hydrocarbon chain. In certain embodiments, L is —CH₂.

In certain embodiments, L is a covalent bond, —CH₂—, —NH—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—.

In some embodiments, L is a bivalent C₂₋₈ straight or branched, hydrocarbon chain wherein L has at least one double bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—.

In certain embodiments, L is a bivalent C₂₋₈ straight or branched, hydrocarbon chain wherein L has at least one double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—.

In some embodiments, L is a bivalent C₂₋₈ straight or branched, hydrocarbon chain wherein L has at least one double bond and at least one methylene unit of L is replaced by —C(O)—, and one or two additional methylene units of L are optionally and independently replaced by cyclopropylene, —O—, —N(R)—, or —C(O)—.

As described above, in certain embodiments, L is a bivalent C₂₋₈ straight or branched, hydrocarbon chain wherein L has at least one double bond. One of ordinary skill in the art will recognize that such a double bond may exist within the hydrocarbon chain backbone or may be “exo” to the backbone chain and thus forming an alkylidene group. By way of example, such an L group having an alkylidene branched chain includes —CH₂C(═CH₂)CH₂—. Thus, in some embodiments, L is a bivalent C₂₋₈ straight or branched, hydrocarbon chain wherein L has at least one alkylidenyl double bond. Exemplary L groups include —NHC(O)C(═CH₂)CH₂—.

In certain embodiments, L is a bivalent C₂₋₈ straight or branched, hydrocarbon chain wherein L has at least one double bond and at least one methylene unit of L is replaced by —C(O)—. In certain embodiments, L is —C(O)CH═CH(CH₃)—, —C(O)CH═CHCH₂NH(CH₃)—, —C(O)CH═CH(CH₃)—, —C(O)CH═CH—, —CH₂C(O)CH═CH—, —CH₂C(O)CH═CH(CH₃)—, —CH₂CH₂C(O)CH═CH—, —CH₂CH₂C(O)CH═CHCH₂—, —CH₂CH₂C(O)CH═CHCH₂NH(CH₃)—, or —CH₂CH₂C(O)CH═CH(CH₃)—, or —CH(CH₃)OC(O)CH═CH—.

In certain embodiments, L is a bivalent C₂₋₈ straight or branched, hydrocarbon chain wherein L has at least one double bond and at least one methylene unit of L is replaced by —OC(O)—.

In some embodiments, L is a bivalent C₂₋₈ straight or branched, hydrocarbon chain wherein L has at least one double bond and at least one methylene unit of L is replaced by —NRC(O)—, —C(O)NR—, —N(R)SO₂—, —SO₂N(R)—, —S—, —S(O)—, —SO₂, —OC(O)—, or —C(O)O—, and one or two additional methylene units of L are optionally and independently replaced by cyclopropylene, —O—, —N(R)—, or —C(O)—. In some embodiments, L is —CH₂OC(O)CH═CHCH₂—, —CH₂—OC(O)CH═CH—, or —CH(CH═CH₂)OC(O)CH═CH—.

In certain embodiments, L is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)C(O)—, —NRC(O)CH═CHCH₂N(CH₃)—, —NRSO₂CH═CH—, —NRSO₂CH═CHCH₂—, —NRC(O)CH═CHCH₂O NRC(O)C(═CH₂)CH₂—, —CH₂NRC(O)—, —CH₂NRC(O)CH═CH—, —CH₂CH₂NRC(O)—, or —CH₂NRC(O)cyclopropylene-, wherein each R is independently hydrogen or optionally substituted C₁₋₆ aliphatic.

In certain embodiments, L is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)C(O)—, —NHC(O)CH═CHCH₂N(CH₃)—, —NHSO₂CH═CH—, —NHSO₂CH═CHCH₂—, —NHC(O)CH═CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-.

In some embodiments, L is a bivalent C₂₋₈ straight or branched, hydrocarbon chain wherein L has at least one triple bond. In certain embodiments, L is a bivalent C₂₋₈ straight or branched, hydrocarbon chain wherein L has at least one triple bond and one or two additional methylene units of L are optionally and independently replaced by —NRC(O)—, —C(O)NR—, —S—, —S(O)—, —SO₂, —C(═S)—, —C(═NR)—, —O—, —N(R), or —C(O)—. In some embodiments, L has at least one triple bond and at least one methylene unit of L is replaced by —N(R)—, —N(R)C(O)—, —C(O)—, —C(O)O—, or —OC(O)—, or —O—.

Exemplary L groups include —C≡C—, —C≡CCH₂N(isopropyl)-, —NHC(O)C≡CCH₂CH₂—, —CH₂—C≡C≡CH₂—, —C≡CCH₂O—, —CH₂C(O)C≡C—, —C(O)C≡C—, or —CH₂OC(═O)C≡C—.

In certain embodiments, L is a bivalent C₂₋₈ straight or branched, hydrocarbon chain wherein one methylene unit of L is replaced by cyclopropylene and one or two additional methylene units of L are independently replaced by —C(O)—, —NRC(O)—, —C(O)NR—, —N(R)SO₂—, or —SO₂N(R)—. Exemplary L groups include —NHC(O)-cyclopropylene-SO₂— and —NHC(O)-cyclopropylene-.

As defined generally above, Y^{W} is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with at 1-4 R^{e} groups, each R^{e} is independently selected from -Q-Z, oxo, NO₂, halogen, CN, a suitable leaving group, or C₁₋₆ aliphatic, wherein Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and, Z is hydrogen or C₁₋₆ aliphatic optionally substituted with oxo, halogen, NO₂, or CN.

In certain embodiments, Y^{W} is hydrogen.

In certain embodiments, Y^{W} is C₁₋₆ aliphatic optionally substituted with oxo, halogen, NO₂, or CN. In some embodiments, Y^{W} is C₂₋₆alkenyl optionally substituted with oxo, halogen, NO₂, or CN. In other embodiments, Y^{W} is C₂₋₆alkynyl optionally substituted with oxo, halogen, NO₂, or CN. In some embodiments, Y^{W} is C₂₋₆alkenyl. In other embodiments, Y^{W} is C₂₋₄ alkynyl.

In other embodiments, Y^{W} is C₁₋₆ alkyl substituted with oxo, halogen, NO₂, or CN. Such Y^{W} groups include —CH₂F, —CH₂Cl, —CH₂CN, and —CH₂NO₂.

In certain embodiments, Y^{W} is a saturated 3-6 membered monocyclic ring having 0-3 heteroatoms independently selected from nitrogen, oxygen, or sulfur, wherein Y^{W} is substituted with 1-4 R^{e} groups, wherein each R^{e} is as defined above and described herein.

In some embodiments, Y^{W} is a saturated 3-4 membered heterocyclic ring having 1 heteroatom selected from oxygen or nitrogen wherein said ring is substituted with 1-2 R^{e} groups, wherein each R^{e} is as defined above and described herein. Exemplary such rings are epoxide and oxetane rings, wherein each ring is substituted with 1-2 R^{e} groups, wherein each R^{e} is as defined above and described herein.

In other embodiments, Y^{W} is a saturated 5-6 membered heterocyclic ring having 1-2 heteroatom selected from oxygen or nitrogen wherein said ring is substituted with 1-4 R^{e} groups, wherein each R^{e} is as defined above and described herein. Such rings include piperidine and pyrrolidine, wherein each ring is substituted with 1-4 R^{e} groups, wherein each R^{e} is as defined above and described herein. In certain embodiments, Y^{W} is

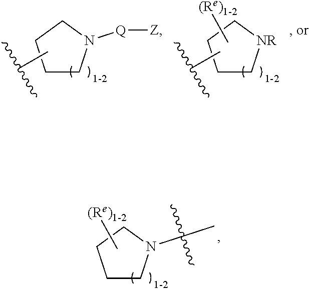

wherein each R, Q, Z, and R^{e} is as defined above and described herein.

In some embodiments, Y^{W} is a saturated 3-6 membered carbocyclic ring, wherein said ring is substituted with 1-4 R^{e} groups, wherein each R^{e} is as defined above and described herein. In certain embodiments, Y^{W} is cyclopropyl, cyclobutyl, cyclopentyl, or cyclohexyl, wherein each ring is substituted with 1-4 R^{e} groups, wherein each R^{e} is as defined above and described herein. In certain embodiments, Y^{W} is

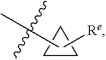

wherein R^{e} is as defined above and described herein.

In certain embodiments, Y^{W} is cyclopropyl optionally substituted with halogen, CN or NO₂.

In certain embodiments, Y^{W} is a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein.

In some embodiments, Y^{W} is a partially unsaturated 3-6 membered carbocyclic ring, wherein said ring is substituted with 1-4 R^{e} groups, wherein each R^{e} is as defined above and described herein. In some embodiments, Y^{W} is cyclopropenyl, cyclobutenyl, cyclopentenyl, or cyclohexenyl wherein each ring is substituted with 1-4 R^{e} groups, wherein each R^{e} is as defined 0-3 above and described herein. In certain embodiments, Y^{W} is

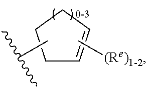

wherein each R^{e} is as defined above and described herein.

In certain embodiments, Y^{W} is a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein. In certain embodiments, Y is selected from:

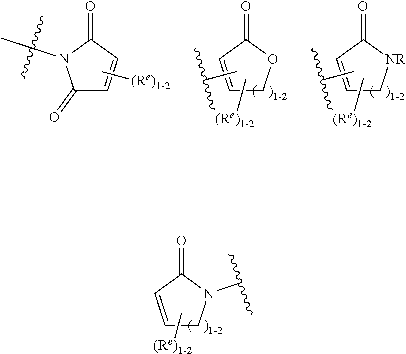

wherein each R and R^{e} is as defined above and described herein.

In certain embodiments, Y^{W} is a 6-membered aromatic ring having 0-2 nitrogens wherein said ring is substituted with 1-4 R^{e} groups, wherein each R^{e} group is as defined above and described herein. In certain embodiments, Y^{W} is phenyl, pyridyl, or pyrimidinyl, wherein each ring is substituted with 1-4 R^{e} groups, wherein each R^{e} is as defined above and described herein.

In some embodiments, Y^{W} is selected from:

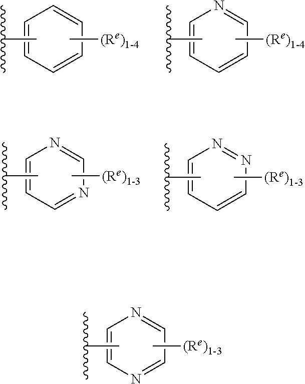

wherein each R^{e} is as defined above and described herein.

In other embodiments, Y^{W} is a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein. In some embodiments, Y^{W} is a 5 membered partially unsaturated or aryl ring having 1-3 heteroatoms independently selected from nitrogen, oxygen, and sulfur, wherein said ring is substituted with 1-4R^{e} groups, wherein each R^{e} group is as defined above and described herein. Exemplary such rings are isoxazolyl, oxazolyl, thiazolyl, imidazolyl, pyrazolyl, pyrrolyl, furanyl, thienyl, triazole, thiadiazole, and oxadiazole, wherein each ring is substituted with 1-3 R^{e} groups, wherein each R^{e} group is as defined above and described herein. In certain embodiments, Y^{W} is selected from:

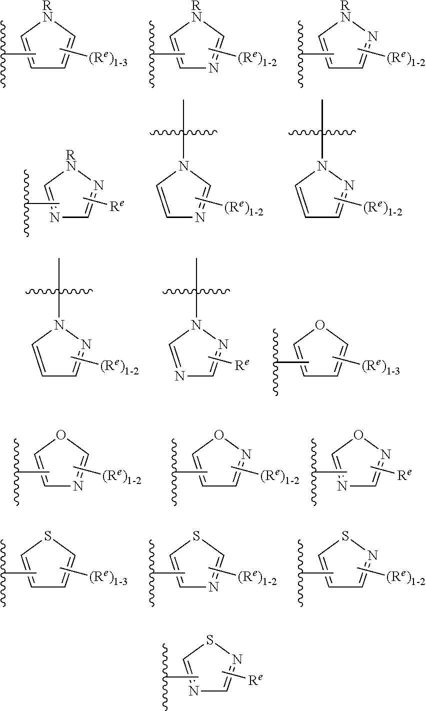

wherein each R and R^{e} is as defined above and described herein.

In certain embodiments, Y^{W} is an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein. According to another aspect, Y^{W} is a 9-10 membered bicyclic, partially unsaturated, or aryl ring having 1-3 heteroatoms independently selected from nitrogen, oxygen, or sulfur, wherein said ring is substituted with 1-4 R^{e} groups, wherein R^{e} is as defined above and described herein. Exemplary such bicyclic rings include 2,3-dihydrobenzo[d]isothiazole, wherein said ring is substituted with 1-4 R^{e} groups, wherein R^{e} is as defined above and described herein.

As defined generally above, each R^{e} group is independently selected from -Q-Z, oxo, NO₂, halogen, CN, a suitable leaving group, or C₁₋₆ aliphatic optionally substituted with oxo, halogen, NO₂, or CN, wherein Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and Z is hydrogen or C₁₋₆ aliphatic optionally substituted with oxo, halogen, NO₂, or CN.

In certain embodiments, R^{e} is C₁₋₆ aliphatic optionally substituted with oxo, halogen, NO₂, or CN. In other embodiments, R^{e} is oxo, NO₂, halogen, or CN.

In some embodiments, R^{e} is -Q-Z, wherein Q is a covalent bond and Z is hydrogen (i.e., R^{e} is hydrogen). In other embodiments, R^{e} is -Q-Z, wherein Q is a bivalent C₁₋₆ saturated or unsaturated, straight or branched, hydrocarbon chain, wherein one or two methylene units of Q are optionally and independently replaced by NR—, —NRC(O)—, —C(O)NR—, —S—, —O—, —C(O)—, —SO—, or —SO₂—. In other embodiments, Q is a bivalent C₂₋₆ straight or branched, hydrocarbon chain having at least one double bond, wherein one or two methylene units of Q are optionally and independently replaced by NR—, —NRC(O)—, —C(O)NR—, —S—, —O—, —C(O)—, —SO—, or —SO₂—. In certain embodiments, the Z moiety of the R^{e} group is hydrogen. In some embodiments, -Q-Z is —NHC(O)CH═CH₂ or —C(O)CH═CH₂.

In certain embodiments, each R^{e} is independently selected from oxo, NO₂, CN, fluoro, chloro, —NHC(O)CH═CH₂, —C(O)CH═CH₂, —CH₂CH═CH₂, —C≡CH, —C(O)OCH₂Cl, —C(O)OCH₂F, —C(O)OCH₂CN, —C(O)CH₂Cl, —C(O)CH₂F, —C(O)CH₂CN, or —CH₂C(O)CH₃.

In certain embodiments, R^{e} is a suitable leaving group, ie a group that is subject to nucleophilic displacement. A “suitable leaving” is a chemical group that is readily displaced by a desired incoming chemical moiety such as the thiol moiety of a cysteine of interest. Suitable leaving groups are well known in the art, e.g., see, “Advanced Organic Chemistry,” Jerry March, 5^{th} Ed., pp. 351-357, John Wiley and Sons, N.Y. Such leaving groups include, but are not limited to, halogen, alkoxy, sulphonyloxy, optionally substituted alkylsulphonyloxy, optionally substituted alkenylsulfonyloxy, optionally substituted arylsulfonyloxy, acyl, and diazonium moieties. Examples of suitable leaving groups include chloro, iodo, bromo, fluoro, acetoxy, methanesulfonyloxy (mesyloxy), tosyloxy, triflyloxy, nitro-phenylsulfonyloxy (nosyloxy), and bromo-phenylsulfonyloxy (brosyloxy).

In certain embodiments, the following embodiments and combinations of -L-Y^{W} apply:
(a) L is a bivalent C₂₋₈ straight or branched, hydrocarbon chain wherein L has at least one double bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—; and Y^{W} is hydrogen or C₁₋₆ aliphatic optionally substituted with oxo, halogen, NO₂, or CN; or
(b) L is a bivalent C₂₋₈ straight or branched, hydrocarbon chain wherein L has at least one double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—; and Y^{W} is hydrogen or C₁₋₆ aliphatic optionally substituted with oxo, halogen, NO₂, or CN; or
(c) L is a bivalent C₂₋₈ straight or branched, hydrocarbon chain wherein L has at least one double bond and at least one methylene unit of L is replaced by —C(O)—, and one or two additional methylene units of L are optionally and independently replaced by cyclopropylene, —O—, —N(R)—, or —C(O)—; and Y^{W} is hydrogen or C₁₋₆ aliphatic optionally substituted with oxo, halogen, NO₂, or CN; or
(d) L is a bivalent C₂₋₈ straight or branched, hydrocarbon chain wherein L has at least one double bond and at least one methylene unit of L is replaced by —C(O)—; and Y^{W} is hydrogen or C₁₋₆ aliphatic optionally substituted with oxo, halogen, NO₂, or CN; or
(e) L is a bivalent C₂₋₈ straight or branched, hydrocarbon chain wherein L has at least one double bond and at least one methylene unit of L is replaced by OC(O)—; and Y^{W} is hydrogen or C₁₋₆ aliphatic optionally substituted with oxo, halogen, NO₂, or CN; or
(f) L is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)—, —NRC(O)(C═N₂)C(O)—, —NRC(O)CH═CHCH₂N(CH₃)—, —NRSO₂CH═CH—, —NRSO₂CH═CHCH₂—, —NRC(O)CH═CHCH₂O—, —NRC(O)C(═CH₂)CH₂—, —CH₂NRC(O)—, —CH₂NRC(O)CH═CH—, —CH₂CH₂NRC(O)—, or —CH₂NRC(O)cyclopropylene-; wherein R is H or optionally substituted C₁₋₆ aliphatic; and Y^{W} is hydrogen or C₁₋₆ aliphatic optionally substituted with oxo, halogen, NO₂, or CN; or
(g) L is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)—, —NHC(O)(C═N₂)C(O)—, —NHC(O)CH═CHCH₂N(CH₃)—, —NHSO₂CH═CH—, —NHSO₂CH═CHCH₂—, —NHC(O)CH═CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-; and Y^{W} is hydrogen or C₁₋₆ aliphatic optionally substituted with oxo, halogen, NO₂, or CN; or
(h) L is a bivalent C₂₋₈ straight or branched, hydrocarbon chain wherein L has at least one alkylidenyl double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—; and Y^{W} is hydrogen or C₁₋₆ aliphatic optionally substituted with oxo, halogen, NO₂, or CN; or
(i) L is a bivalent C₂₋₈ straight or branched, hydrocarbon chain wherein L has at least one triple bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, or —C(O)O—, and Y^{W} is hydrogen or C₁₋₆ aliphatic optionally substituted with oxo, halogen, NO₂, or CN; or
(j) L is —C≡C—, —C≡CCH₂N(isopropyl)-, —NHC(O)C≡CCH₂CH₂—, —CH₂—C≡C≡CH₂, —C≡CCH₂O—, —CH₂C(O)C≡C—, —C(O)C≡C—, or —CH₂C(═O)C≡C; and Y^{W} is hydrogen or C₁₋₆ aliphatic optionally substituted with oxo, halogen, NO₂, or CN; or
(k) L is a bivalent C₂₋₈ straight or branched, hydrocarbon chain wherein one methylene unit of L is replaced by cyclopropylene and one or two additional methylene units of L are independently replaced by —NRC(O)—, —C(O)NR—, —N(R)SO₂—, —SO₂N(R)—, —S—, —S(O)—, —SO₂—, —OC(O)—, or —C(O)O—; and Y^{W} is hydrogen or C₁₋₆ aliphatic optionally substituted with oxo, halogen, NO₂, or CN; or
(l) L is a covalent bond and Y^{W} is selected from:
   (i) C₁₋₆ alkyl substituted with oxo, halogen, NO₂, or CN;
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

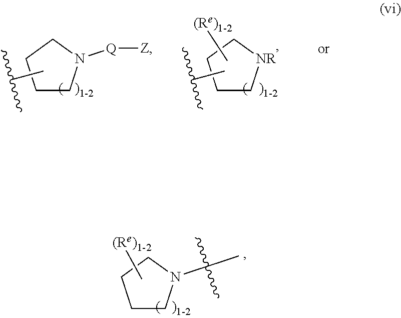

wherein each R, Q, Z, and R^{e} is as defined above and described herein; or
   (vii) a saturated 3-6 membered carbocyclic ring, wherein said ring is substituted with 1-4 R^{e} groups, wherein each R^{e} is as defined above and described herein; or
   (viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
   (ix) a partially unsaturated 3-6 membered carbocyclic ring, wherein said ring is substituted with 1-4 R^{e} groups, wherein each R^{e} is as defined above and described herein; or

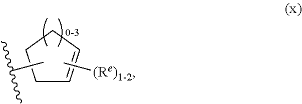

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

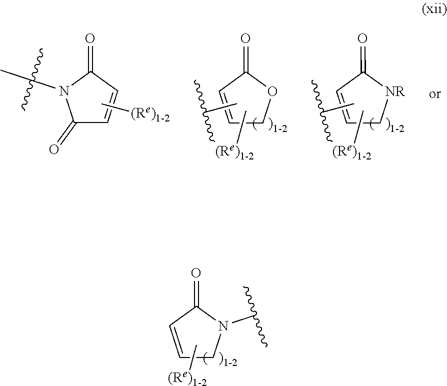

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

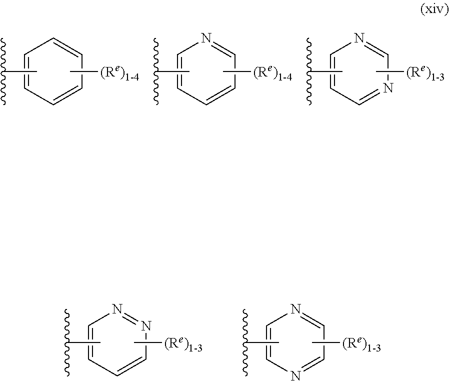

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

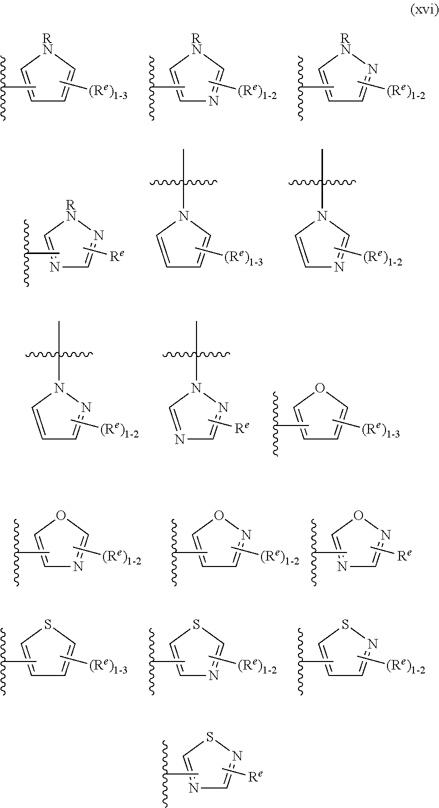

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(m) L is C(O) and Y^{W} is selected from:
   (i) C₁₋₆ alkyl substituted with oxo, halogen, NO₂, or CN; or
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

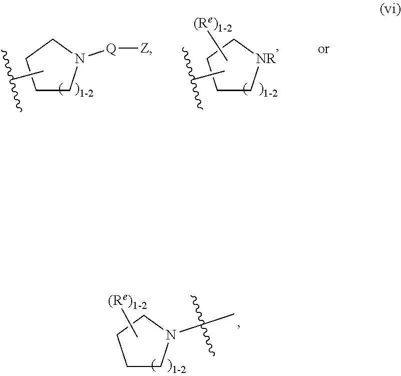

wherein each R, Q, Z, and R^{e} is as defined above and described herein; or
   (vii) a saturated 3-6 membered carbocyclic ring, wherein said ring is substituted with 1-4 R^{e} groups, wherein each R^{e} is as defined above and described herein; or
   (viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
   (ix) a partially unsaturated 3-6 membered carbocyclic ring, wherein said ring is substituted with 1-4 R^{e} groups, wherein each R^{e} is as defined above and described herein; or

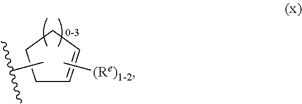

wherein each R^{e} is as defined above and described herein; or
   (xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

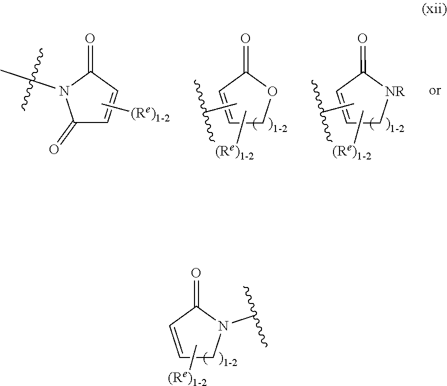

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

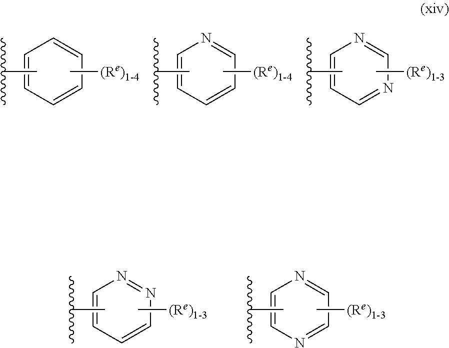

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

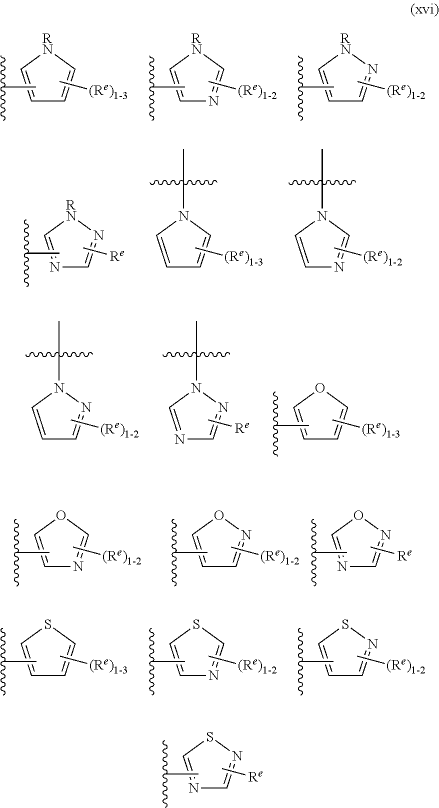

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(n) L is N(R)C(O) and Y^{W} is selected from:
   (i) C₁₋₆ alkyl substituted with oxo, halogen, NO₂, or CN; or
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

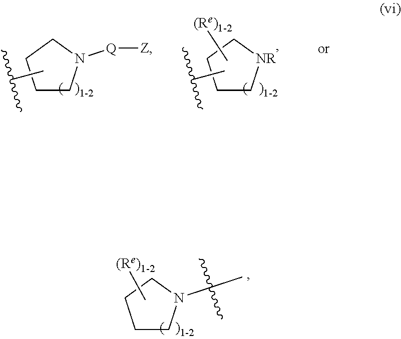

wherein each R, Q, Z, and R^{e} is as defined above and described herein; or
   (vii) a saturated 3-6 membered carbocyclic ring, wherein said ring is substituted with 1-4 R^{e} groups, wherein each R^{e} is as defined above and described herein; or
   (viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
   (ix) a partially unsaturated 3-6 membered carbocyclic ring, wherein said ring is substituted with 1-4 R^{e} groups, wherein each R^{e} is as defined above and described herein; or

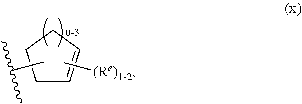

wherein each R^{e} is as defined above and described herein; or
   (xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

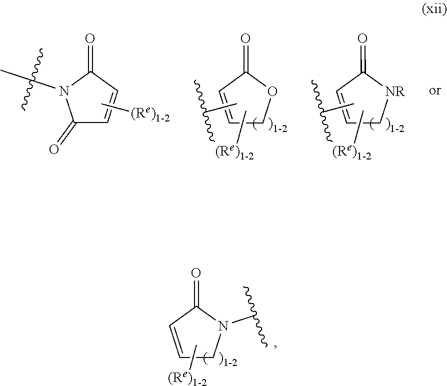

wherein each R and R^{e} is as defined above and described herein; or
   (xiii) a 6-membered aromatic ring having 0-2 nitrogens wherein said ring is substituted with 1-4 R^{e} groups, wherein each R^{e} group is as defined above and described herein; or

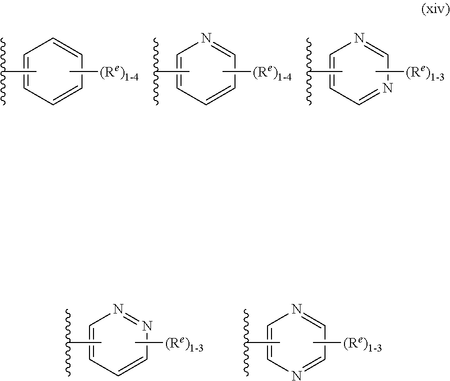

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

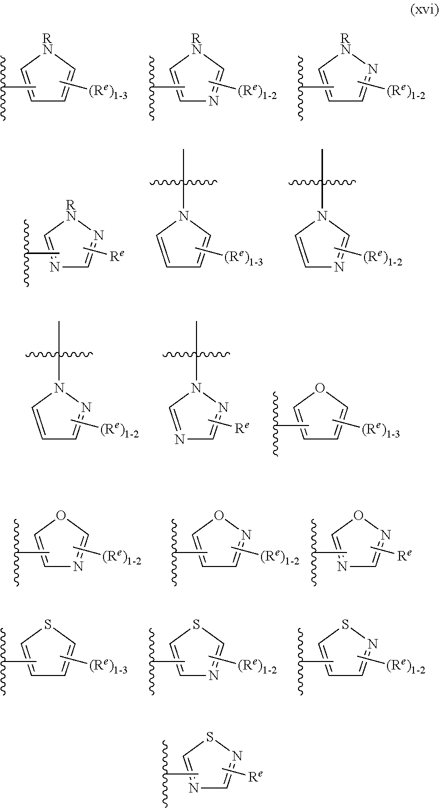

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(o) L is a bivalent C₁₋₈ saturated or unsaturated, straight or branched, hydrocarbon chain; and Y^{W} is selected from:
   (i) C₁₋₆ alkyl substituted with oxo, halogen, NO₂, or CN;
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

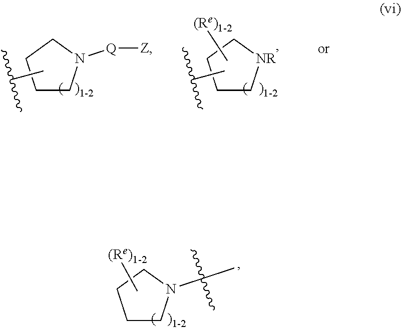

wherein each R, Q, Z, and R^{e} is as defined above and described herein; or
   (vii) a saturated 3-6 membered carbocyclic ring, wherein said ring is substituted with 1-4 R^{e} groups, wherein each R^{e} is as defined above and described herein; or
   (viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
   (ix) a partially unsaturated 3-6 membered carbocyclic ring, wherein said ring is substituted with 1-4 R^{e} groups, wherein each R^{e} is as defined above and described herein; or

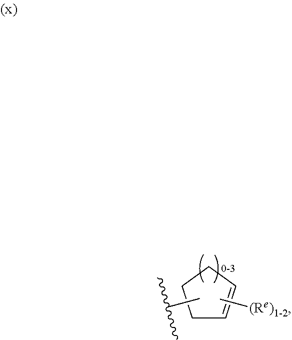

wherein each R^{e} is as defined above and described herein; or
   (xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

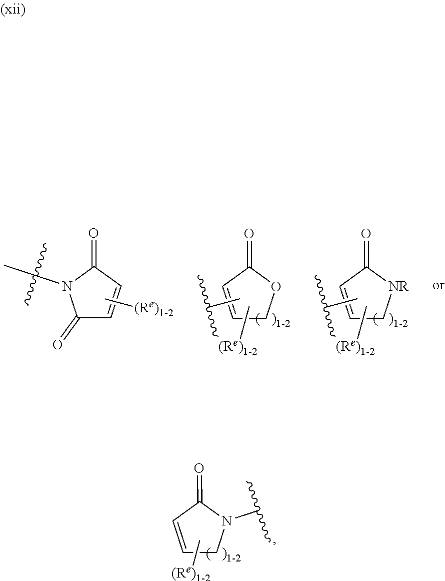

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

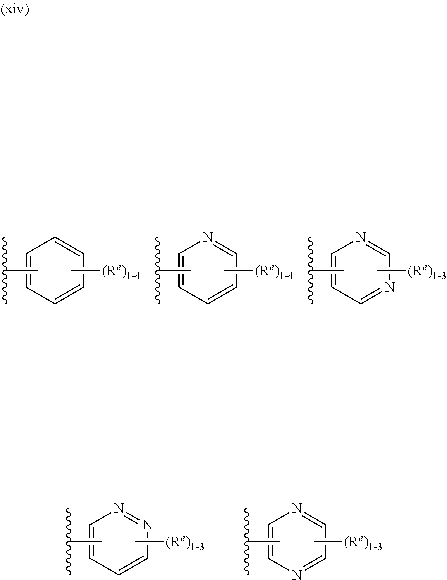

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

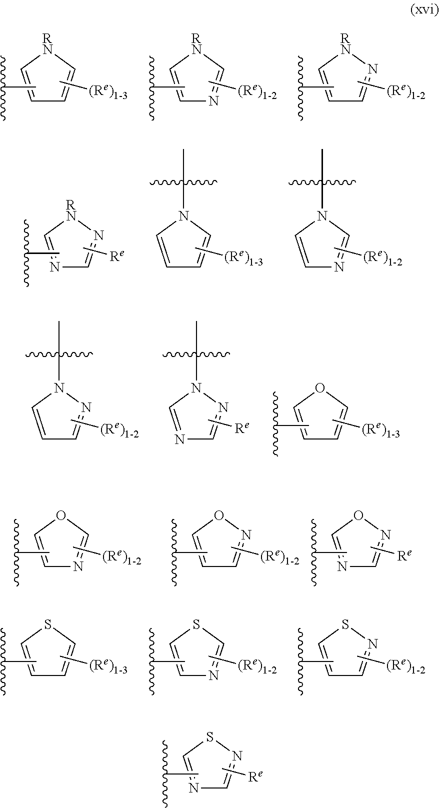

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(p) L is a covalent bond, —CH₂—, —NH—, —C(O)—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—; and Y^{W} is selected from:
   (i) C₁₋₆ alkyl substituted with oxo, halogen, NO₂, or CN; or
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

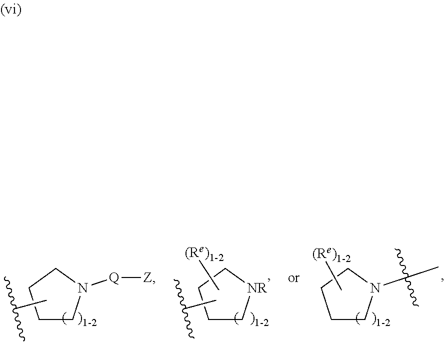

wherein each R, Q, Z, and R^{e} is as defined above and described herein; or
   (vii) a saturated 3-6 membered carbocyclic ring, wherein said ring is substituted with 1-4 R^{e} groups, wherein each R^{e} is as defined above and described herein; or
   (viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
   (ix) a partially unsaturated 3-6 membered carbocyclic ring, wherein said ring is substituted with 1-4 R^{e} groups, wherein each R^{e} is as defined above and described herein; or

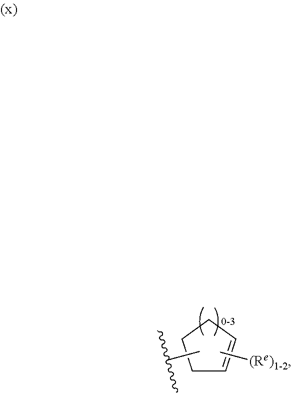

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

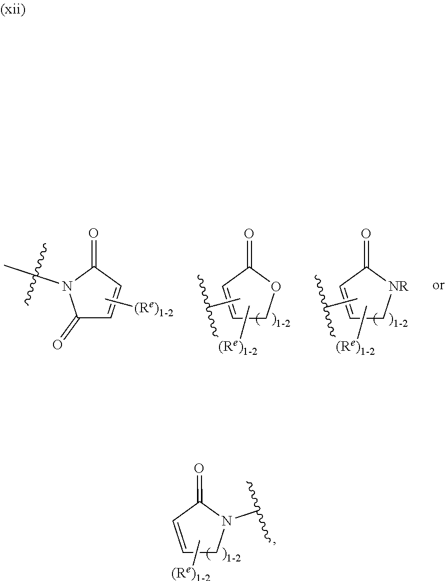

wherein each R and R^{e} is as defined above and described herein; or
   (xiii) a 6-membered aromatic ring having 0-2 nitrogens wherein said ring is substituted with 1-4 R^{e} groups, wherein each R^{e} group is as defined above and described herein; or

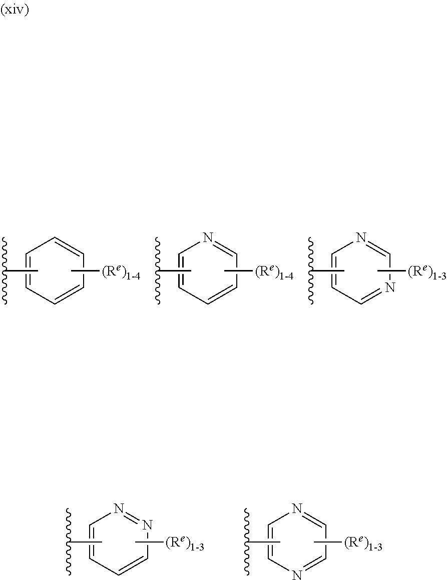

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

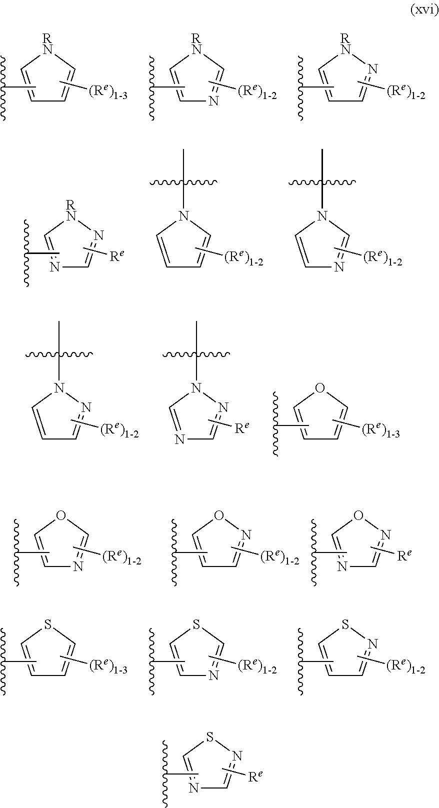

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein.

In certain embodiments, the Y^{W} group is selected from those set forth in Table 1 below, wherein each wavy line indicates the point of attachment to the rest of the molecule.

TABLE 1
Exemplary Y^{W} groups

| | |
|---|---|
| 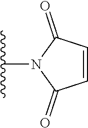 [SMILES: *N1C(=O)C=CC1=O] | a |
| 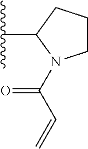 [SMILES: *C1CCCN1C(=O)C=C] | b |
| 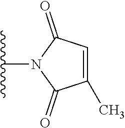 [SMILES: *N1C(=O)C=C(C)C1=O] | c |
| 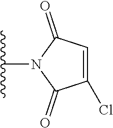 [SMILES: *N1C(=O)C=C(Cl)C1=O] | d |
| 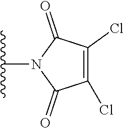 [SMILES: *N1C(=O)C(Cl)=C(Cl)C1=O] | e |
| 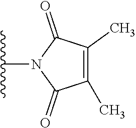 [SMILES: *N1C(=O)C(C)=C(C)C1=O] | f |
| 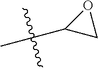 [SMILES: *C1CO1] | g |
| 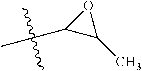 [SMILES: *C1OC1C] | h |
| 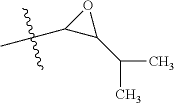 [SMILES: *C1OC1C(C)C] | i |
| 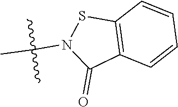 [SMILES: *n1sc2ccccc2c1=O] | j |
| 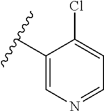 [SMILES: *c1cnccc1Cl] | k |
| 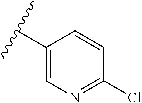 [SMILES: *c1ccc(Cl)nc1] | l |
| 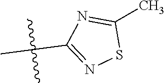 [SMILES: *c1nsc(C)n1] | m |
| 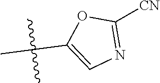 [SMILES: *c1cnc(C#N)o1] | n |
| 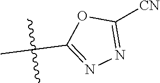 [SMILES: *c1nnc(C#N)o1] | o |
| 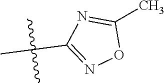 [SMILES: *c1noc(C)n1] | p |
| 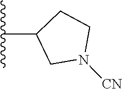 [SMILES: *C1CCN(C#N)C1] | q |
| 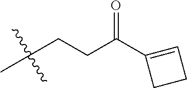 [SMILES: *CCC(=O)C1=CCC1] | r |
| 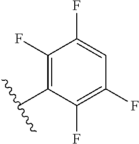 [SMILES: *c1c(F)c(F)cc(F)c1F] | s |
| 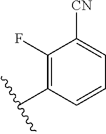 [SMILES: *c1cccc(C#N)c1F] | t |
| 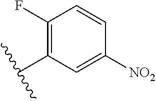 [SMILES: *c1cc([N+](=O)[O-])ccc1F] | u |
| 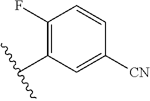 [SMILES: *c1cc(C#N)ccc1F] | v |
| 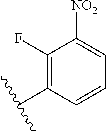 [SMILES: *c1cccc([N+](=O)[O-])c1F] | w |
| 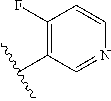 [SMILES: *c1cnccc1F] | x |
| 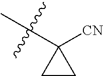 [SMILES: *C1(C#N)CC1] | y |
| 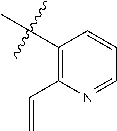 [SMILES: *c1cccnc1C=C] | z |
| 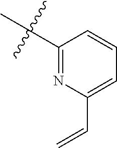 [SMILES: *c1cccc(C=C)n1] | aa |
| 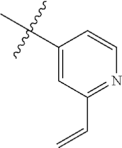 [SMILES: *c1ccnc(C=C)c1] | bb |
| 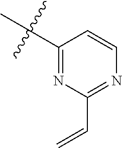 [SMILES: *c1ccnc(C=C)n1] | cc |
| 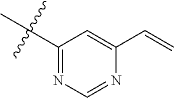 [SMILES: *c1cc(C=C)ncn1] | dd |
| 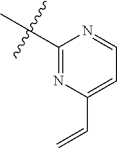 [SMILES: *c1nccc(C=C)n1] | ee |
| 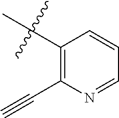 [SMILES: *c1cccnc1C#C] | ff |
| 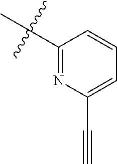 [SMILES: *c1cccc(C#C)n1] | gg |
| 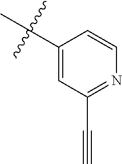 [SMILES: *c1ccnc(C#C)c1] | hh |
| 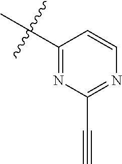 [SMILES: *c1ccnc(C#C)n1] | ii |
| 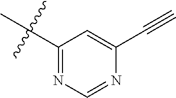 [SMILES: *c1cc(C#C)ncn1] | jj |
| 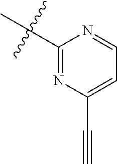 [SMILES: *c1nccc(C#C)n1] | kk |
| 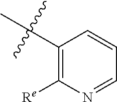 | ll |
| 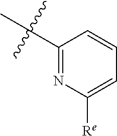 | mm |
| 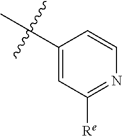 | nn |
| 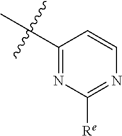 | oo |
| 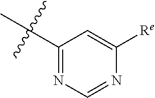 | pp |
| 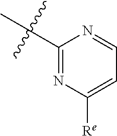 | qq |
| 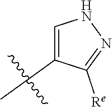 | rr |
| 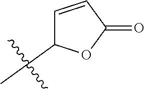 [SMILES: *C1C=CC(=O)O1] | ss |
| 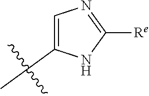 | tt |
| 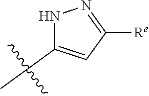 | uu |
| 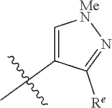 | vv |
| 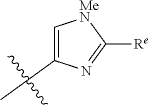 | ww |
| 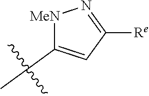 | xx |
| 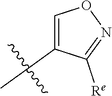 | yy |
| 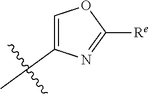 | zz |
| 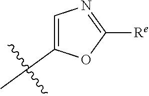 | aaa |
| 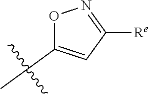 | bbb |
| 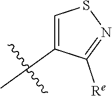 | ccc |
| 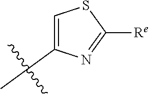 | ddd |
| 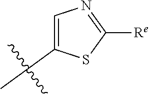 | eee |
| 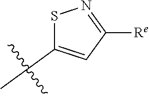 | fff |
| 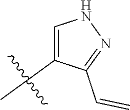 [SMILES: *c1c[nH]nc1C=C] | ggg |
| 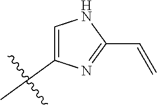 [SMILES: *c1c[nH]c(C=C)n1] | hhh |
| 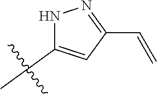 [SMILES: *c1cc(C=C)n[nH]1] | iii |
| 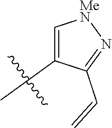 [SMILES: *c1cn(C)nc1C=C] | jjj |
| 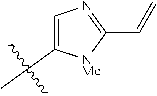 [SMILES: *c1cnc(C=C)n1C] | kkk |
| 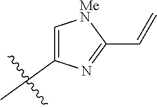 [SMILES: *c1cn(C)c(C=C)n1] | lll |
| 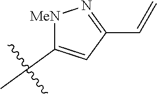 [SMILES: *c1cc(C=C)nn1C] | mmm |
| 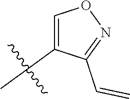 [SMILES: *c1conc1C=C] | nnn |
| 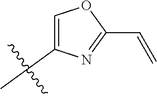 [SMILES: *c1coc(C=C)n1] | ooo |
| 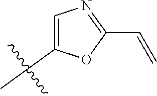 [SMILES: *c1cnc(C=C)o1] | ppp |
| 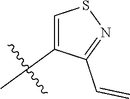 [SMILES: *c1csnc1C=C] | qqq |
| 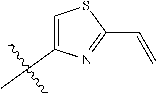 [SMILES: *c1csc(C=C)n1] | rrr |
| 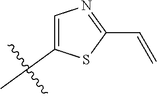 [SMILES: *c1cnc(C=C)s1] | sss |
| 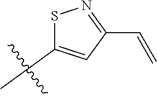 [SMILES: *c1cc(C=C)ns1] | ttt |
| 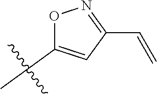 [SMILES: *c1cc(C=C)no1] | uuu |
| 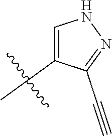 [SMILES: *c1c[nH]nc1C#C] | vvv |
| 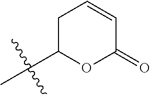 [SMILES: *C1CC=CC(=O)O1] | qqq |
| 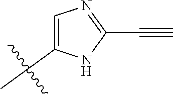 [SMILES: *c1cnc(C#C)[nH]1] | www |
| 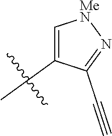 [SMILES: *c1cn(C)nc1C#C] | xxx |
| 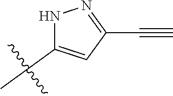 [SMILES: *c1cc(C#C)n[nH]1] | yyy |
| 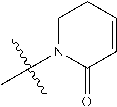 [SMILES: *N1CCC=CC1=O] | zzz |
| 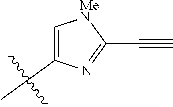 [SMILES: *c1cn(C)c(C#C)n1] | aaaa |
| 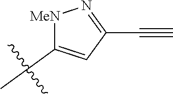 [SMILES: *c1cc(C#C)nn1C] | bbbb |
| 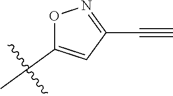 [SMILES: *c1cc(C#C)no1] | cccc |
| 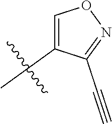 [SMILES: *c1conc1C#C] | dddd |
| 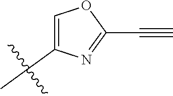 [SMILES: *c1coc(C#C)n1] | eeee |
| 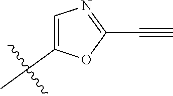 [SMILES: *c1cnc(C#C)o1] | ffff |
| 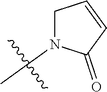 [SMILES: *N1CC=CC1=O] | gggg |
| 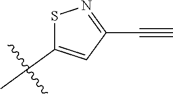 [SMILES: *c1cc(C#C)ns1] | hhhh |
| 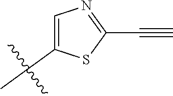 [SMILES: *c1cnc(C#C)s1] | iiii |
| 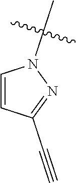 [SMILES: *n1ccc(C#C)n1] | jjjj |
| 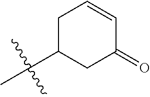 [SMILES: *C1CC=CC(=O)C1] | kkkk |
| 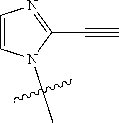 [SMILES: *n1ccnc1C#C] | llll |
| 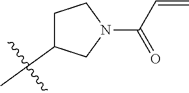 [SMILES: *C1CCN(C(=O)C=C)C1] | mmmm |
| 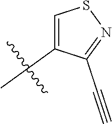 [SMILES: *c1csnc1C#C] | nnnn |
| 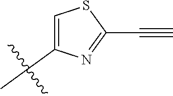 [SMILES: *c1csc(C#C)n1] | oooo |
| 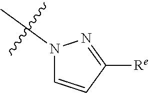 | pppp |
| 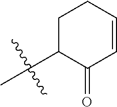 [SMILES: *C1CCC=CC1=O] | qqqq |
| 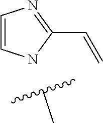 | rrrr |
| 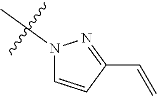 [SMILES: *n1ccc(C=C)n1] | ssss |
| 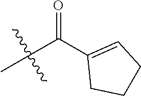 [SMILES: *C(=O)C1=CCCC1] | tttt |
| 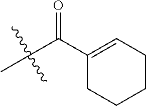 [SMILES: *C(=O)C1=CCCCC1] | uuuu |
| 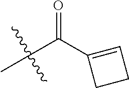 [SMILES: *C(=O)C1=CCC1] | vvvv |
| 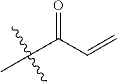 [SMILES: *C(=O)C=C] | wwww |
| 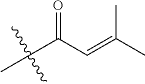 [SMILES: *C(=O)C=C(C)C] | xxxx |
| 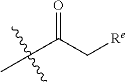 | yyyy |
| 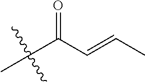 [SMILES: *C(=O)/C=C/C] | zzzz |
| 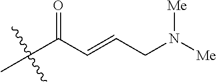 [SMILES: *C(=O)/C=C/CN(C)C] | aaaaa |
| 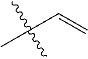 [SMILES: *C=C] | bbbbb |
| 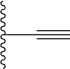 [SMILES: *C#C] | ccccc |

wherein each R^{e} is independently a suitable leaving group, NO₂, CN or oxo.

In certain embodiments, R⁶ is —C≡CH, —C≡CCH₂NH(isopropyl), —NHC(O)C≡CCH₂CH₃, —CH₂—C≡C≡CH₃, —C≡CCH₂OH, CH₂C(O)C≡CH, —C(O)C≡CH, or —CH₂C(═O)C≡CH. In some embodiments, R¹ is selected from —NHC(O)CH═CH₂, —NHC(O)CH═CHCH₂N(CH₃)₂, or —CH₂NHC(O)CH═CH₂.

In certain embodiments, R⁶ is selected from those set forth in Table 2, below, wherein each wavy line indicates the point of attachment to the rest of the molecule.

TABLE 2
Exemplary R6 Groups

| | |
|---|---|
| 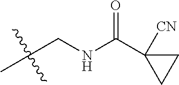 [SMILES: *CNC(=O)C1(C#N)CC1] | a |
| 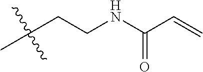 [SMILES: *CCNC(=O)C=C] | b |
| 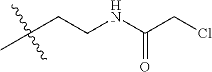 [SMILES: *CCNC(=O)CCl] | c |
| 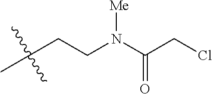 [SMILES: *CCN(C)C(=O)CCl] | d |
| 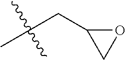 [SMILES: *CC1CO1] | e |
| 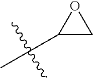 [SMILES: *C1CO1] | f |
| 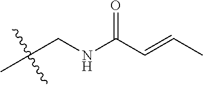 [SMILES: *CNC(=O)/C=C/C] | g |
| 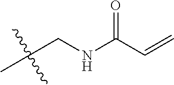 [SMILES: *CNC(=O)C=C] | h |
| 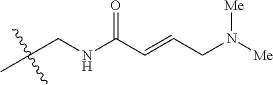 [SMILES: *CNC(=O)/C=C/CN(C)C] | i |
| 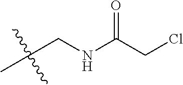 [SMILES: *CNC(=O)CCl] | j |
| 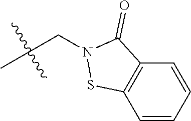 [SMILES: *Cn1sc2ccccc2c1=O] | k |
| 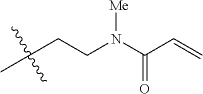 [SMILES: *CCN(C)C(=O)C=C] | l |
| 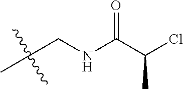 [SMILES: *CNC(=O)[C@H](C)Cl] | m |
| 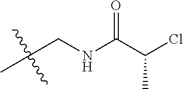 [SMILES: *CNC(=O)[C@@H](C)Cl] | n |
| 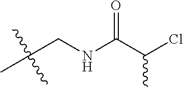 [SMILES: *CNC(=O)C(C)Cl] | o |
| 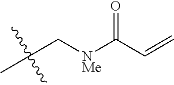 [SMILES: *CN(C)C(=O)C=C] | p |
| 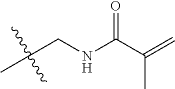 [SMILES: *CNC(=O)C(=C)C] | q |
| 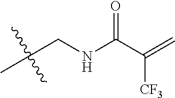 [SMILES: *CNC(=O)C(=C)C(F)(F)F] | r |
| 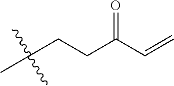 [SMILES: *CCC(=O)C=C] | s |
| 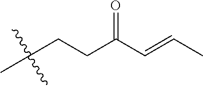 [SMILES: *CCC(=O)/C=C/C] | t |
| 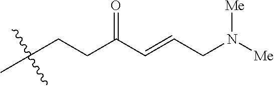 [SMILES: *CCC(=O)/C=C/CN(C)C] | u |
| 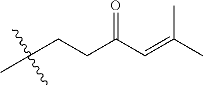 [SMILES: *CCC(=O)C=C(C)C] | v |
| 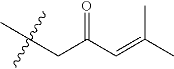 [SMILES: *CC(=O)C=C(C)C] | w |
| 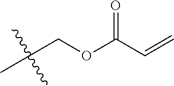 [SMILES: *COC(=O)C=C] | x |
| 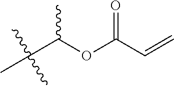 [SMILES: *C(C)OC(=O)C=C] | y |
| 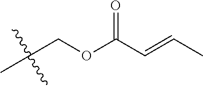 [SMILES: *COC(=O)/C=C/C] | z |
| 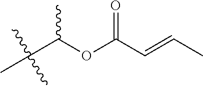 [SMILES: *C(C)OC(=O)/C=C/C] | aa |
| 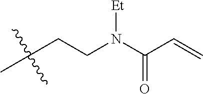 [SMILES: *CCN(CC)C(=O)C=C] | bb |
| 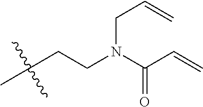 [SMILES: *CCN(CC=C)C(=O)C=C] | cc |
| 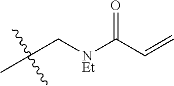 | dd |
| 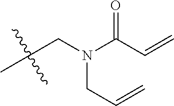 [SMILES: *CN(CC=C)C(=O)C=C] | ee |
| 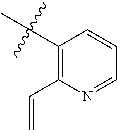 [SMILES: *c1cccnc1C=C] | ff |
| 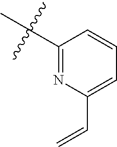 [SMILES: *c1cccc(C=C)n1] | gg |
| 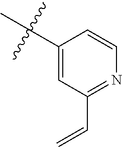 [SMILES: *c1ccnc(C=C)c1] | hh |
| 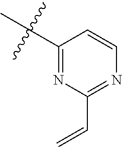 [SMILES: *c1ccnc(C=C)n1] | ii |
| 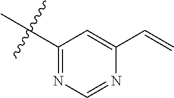 [SMILES: *c1cc(C=C)ncn1] | jj |
| 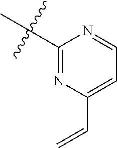 [SMILES: *c1nccc(C=C)n1] | kk |
| 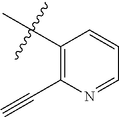 [SMILES: *c1cccnc1C#C] | ll |
| 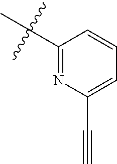 [SMILES: *c1cccc(C#C)n1] | mm |
| 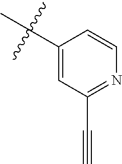 [SMILES: *c1ccnc(C#C)c1] | nn |
| 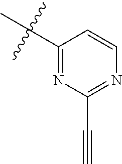 [SMILES: *c1ccnc(C#C)n1] | oo |
| 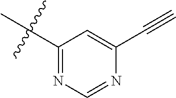 [SMILES: *c1cc(C#C)ncn1] | pp |
| 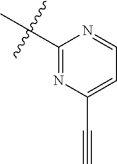 [SMILES: *c1nccc(C#C)n1] | qq |
| 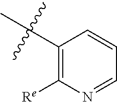 | rr |
| 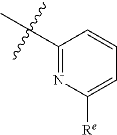 | ss |
| 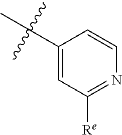 | tt |
| 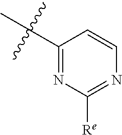 | uu |
| 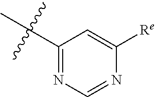 | vv |
| 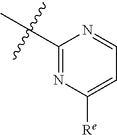 | ww |
| 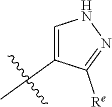 | xx |
| 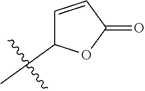 [SMILES: *C1C=CC(=O)O1] | yy |
| 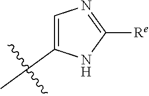 | zz |
| 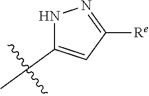 | aaa |
| 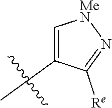 | bbb |
| 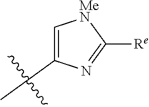 | ccc |
| 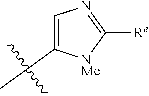 | ddd |
| 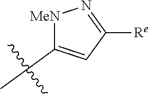 | eee |
| 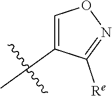 | fff |
| 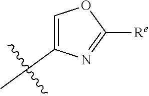 | ggg |
| 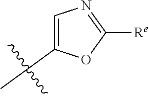 | hhh |
| 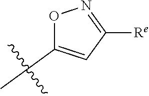 | iii |
| 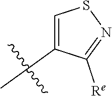 | jjj |
| 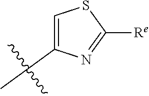 | kkk |
| 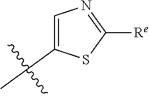 | lll |
| 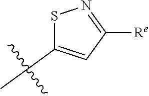 | mmm |
| 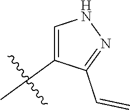 [SMILES: *c1c[nH]nc1C=C] | nnn |
| 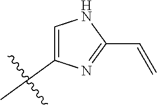 [SMILES: *c1c[nH]c(C=C)n1] | ooo |
| 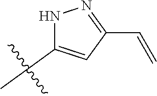 [SMILES: *c1cc(C=C)n[nH]1] | ppp |
| 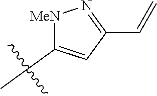 [SMILES: *c1cc(C=C)nn1C] | |
| 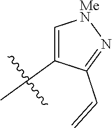 [SMILES: *c1cn(C)nc1C=C] | qqq |
| 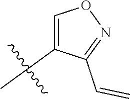 [SMILES: *c1conc1C=C] | rrr |
| 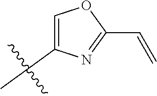 [SMILES: *c1coc(C=C)n1] | sss |
| 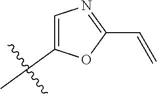 [SMILES: *c1cnc(C=C)o1] | ttt |
| 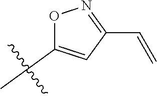 [SMILES: *c1cc(C=C)no1] | uuu |
| 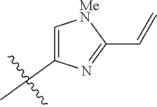 [SMILES: *c1cn(C)c(C=C)n1] | vvv |
| 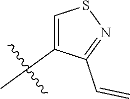 [SMILES: *c1csnc1C=C] | www |
| 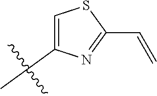 [SMILES: *c1csc(C=C)n1] | xxx |
| 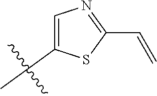 [SMILES: *c1cnc(C=C)s1] | yyy |
| 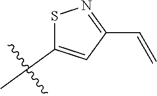 [SMILES: *c1cc(C=C)ns1] | zzz |
| 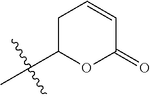 [SMILES: *C1CC=CC(=O)O1] | aaaa |
| 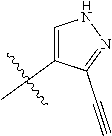 [SMILES: *c1c[nH]nc1C#C] | bbbb |
| 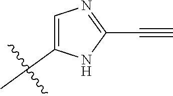 [SMILES: *c1cnc(C#C)[nH]1] | cccc |
| 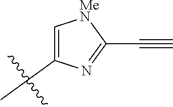 [SMILES: *c1cn(C)c(C#C)n1] | dddd |
| 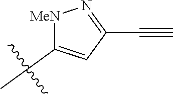 [SMILES: *c1cc(C#C)nn1C] | eeee |
| 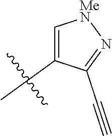 [SMILES: *c1cn(C)nc1C#C] | ffff |
| 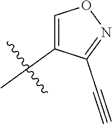 [SMILES: *c1conc1C#C] | gggg |
| 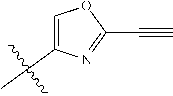 [SMILES: *c1coc(C#C)n1] | hhhh |
| 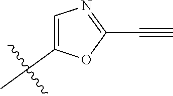 [SMILES: *c1cnc(C#C)o1] | iiii |
| 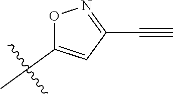 [SMILES: *c1cc(C#C)no1] | jjjj |
| 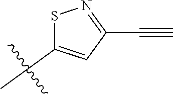 [SMILES: *c1cc(C#C)ns1] | kkkk |
| 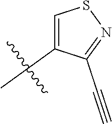 [SMILES: *c1csnc1C#C] | llll |
| 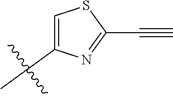 [SMILES: *c1csc(C#C)n1] | mmmm |
| 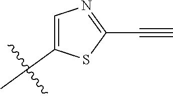 [SMILES: *c1cnc(C#C)s1] | nnnn |
| 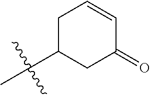 [SMILES: *C1CC=CC(=O)C1] | oooo |
| 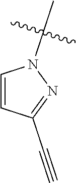 [SMILES: *n1ccc(C#C)n1] | pppp |
| 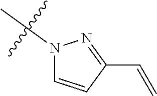 [SMILES: *n1ccc(C=C)n1] | qqqq |
| 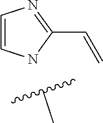 | rrrr |
| 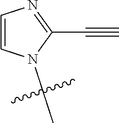 [SMILES: *n1ccnc1C#C] | ssss |
| 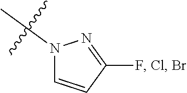 | tttt |
| 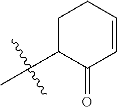 [SMILES: *C1CCC=CC1=O] | uuuu |
| 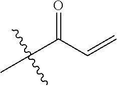 [SMILES: *C(=O)C=C] | vvvv |
| 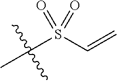 [SMILES: *S(=O)(=O)C=C] | wwww |
| 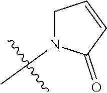 [SMILES: *N1CC=CC1=O] | xxxx |
| 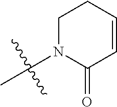 [SMILES: *N1CCC=CC1=O] | yyyy |
| 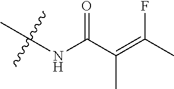 [SMILES: *NC(=O)/C(C)=C(/C)F] | zzzz |
| 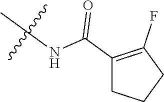 [SMILES: *NC(=O)C1=C(F)CCC1] | aaaaa |
| 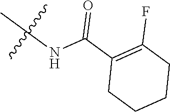 [SMILES: *NC(=O)C1=C(F)CCCC1] | bbbbb |
| 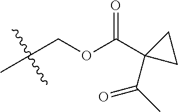 [SMILES: *COC(=O)C1(C(C)=O)CC1] | ccccc |
| 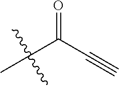 [SMILES: *C(=O)C#C] | ddddd |
| 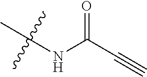 [SMILES: *NC(=O)C#C] | eeeee |
| 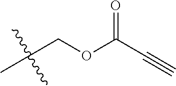 [SMILES: *COC(=O)C#C] | fffff |
| 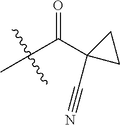 [SMILES: *C(=O)C1(C#N)CC1] | ggggg |
| 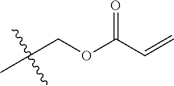 [SMILES: *COC(=O)C=C] | hhhhh |
| 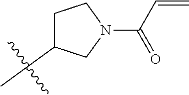 [SMILES: *C1CCN(C(=O)C=C)C1] | iiiii |
| 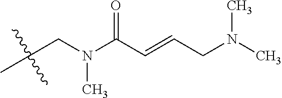 [SMILES: *CN(C)C(=O)/C=C/CN(C)C] | jjjjj |
| 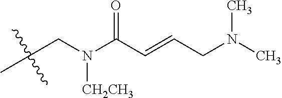 [SMILES: *CN(CC)C(=O)/C=C/CN(C)C] | kkkkk |
| 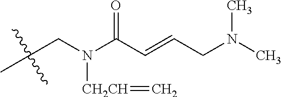 | lllll |
| 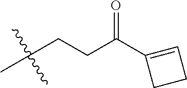 [SMILES: *CCC(=O)C1=CCC1] | mmmmm |
| 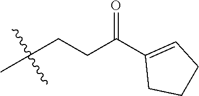 [SMILES: *CCC(=O)C1=CCCC1] | nnnnn |
| 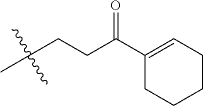 [SMILES: *CCC(=O)C1=CCCCC1] | ooooo |
| 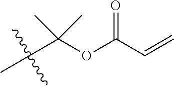 [SMILES: *C(C)(C)OC(=O)C=C] | ppppp |
| 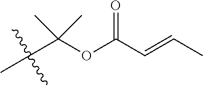 [SMILES: *C(C)(C)OC(=O)/C=C/C] | qqqqq |
| 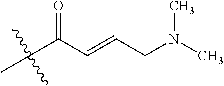 [SMILES: *C(=O)/C=C/CN(C)C] | rrrrr |
| 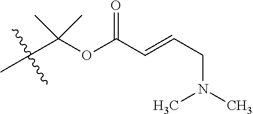 [SMILES: *C(C)(C)OC(=O)/C=C/CN(C)C] | sssss |
| 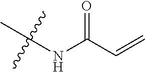 [SMILES: *NC(=O)C=C] | ttttt |
| 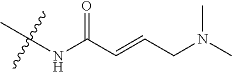 [SMILES: *NC(=O)/C=C/CN(C)C] | uuuuu |
| 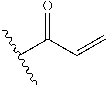 [SMILES: *C(=O)C=C] | vvvvv |
| 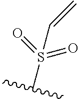 [SMILES: *S(=O)(=O)C=C] | wwwww |
| 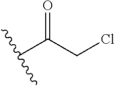 [SMILES: *C(=O)CCl] | xxxxx |
| 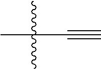 [SMILES: *C#C] | yyyyy |
| 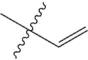 [SMILES: *C=C] | zzzzz |
| 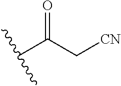 [SMILES: *C(=O)CC#N] | aaaaaa |
| 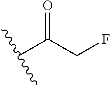 [SMILES: *C(=O)CF] | bbbbbb |
| 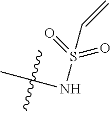 [SMILES: *NS(=O)(=O)C=C] | cccccc |
| 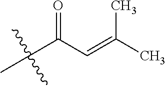 [SMILES: *C(=O)C=C(C)C] | dddddd |
| 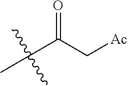 [SMILES: *C(=O)CC(C)=O] | eeeeee |
| 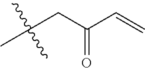 [SMILES: *CC(=O)C=C] | ffffff |
| 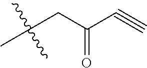 [SMILES: *CC(=O)C#C] | gggggg |
| 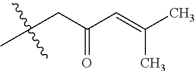 [SMILES: *CC(=O)C=C(C)C] | hhhhhh |
| 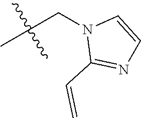 [SMILES: *Cn1ccnc1C=C] | iiiiiii |
| 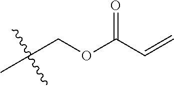 [SMILES: *COC(=O)C=C] | jjjjjj |
| 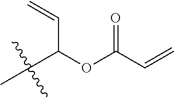 [SMILES: *C(C=C)OC(=O)C=C] | kkkkkk |
| 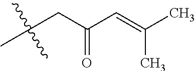 [SMILES: *CC(=O)C=C(C)C] | llllll |
| 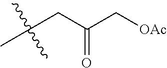 [SMILES: *CC(=O)COC(C)=O] | mmmmmm |
| 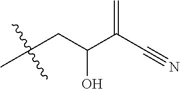 [SMILES: *CC(O)C(=C)C#N] | nnnnnn |
| 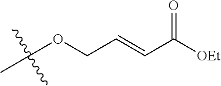 [SMILES: *OC/C=C/C(=O)OCC] | oooooo |
| 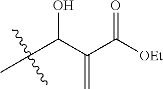 [SMILES: *C(O)C(=C)C(=O)OCC] | pppppp |
| 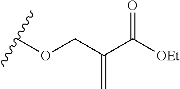 [SMILES: *OCC(=C)C(=O)OCC] | qqqqqq |
| 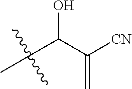 [SMILES: *C(O)C(=C)C#N] | rrrrrr |
| 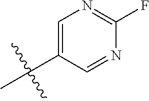 [SMILES: *c1cnc(F)nc1] | ssssss |
| 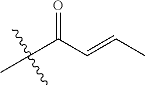 [SMILES: *C(=O)/C=C/C] | tttttt |
| 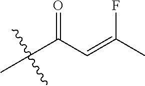 [SMILES: *C(=O)/C=C(/C)F] | uuuuuu |
| 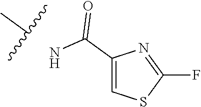 | vvvvvv |
| 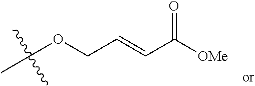 [SMILES: *OC/C=C/C(=O)OC] | wwwwww |
| 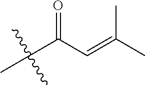 [SMILES: *C(=O)C=C(C)C] | xxxxxx |

wherein each R^{e} is independently a suitable leaving group, NO₂, CN or oxo.

As defined generally above, R⁶ comprises or is a warhead group. Without wishing to be bound by any particular theory, it is believed that such R⁶ groups, i.e. warhead groups, are particularly suitable for covalently binding to the sulfhydryl side chain moiety of Cys277 of Src.

Thus, in some embodiments, R⁶ is characterized in that the -L-Y^{W} moiety is capable of covalently binding to a cysteine residue thereby irreversibly inhibiting the enzyme. In certain embodiments, the cysteine residue is Cys277 of Src.

As defined generally above, R^{6A} is hydrogen, halogen, —CN, —C(O)R, —C(O)OR, —C(O)NR₂, —C(O)N(R)OR, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{6A} is halogen, —CN, —C(O)R, —C(O)OR, —C(O)NR₂, —C(O)N(R)OR, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{6A} is hydrogen, halogen, —CN, —C(O)R, —C(O)OR, —C(O)NR₂, or —C(O)N(R)OR. In some embodiments, R^{6A} is halogen, —CN, —C(O)R, —C(O)OR, —C(O)NR₂, or —C(O)N(R)OR. In some embodiments, R^{6A} is an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{6A} is hydrogen. In some embodiments, R^{6A} is halogen. In some embodiments, R^{6A} is —CN. In some embodiments, R^{6A} is —C(O)R. In some embodiments, R^{6A} is —C(O)OR. In some embodiments, R^{6A} is —C(O)NR₂. In some embodiments, R^{6A} is —C(O)N(R)OR. In some embodiments, R^{6A} is an optionally substituted C₁₋₆ aliphatic. In some embodiments, R^{6A} is an optionally substituted phenyl. In some embodiments, R^{6A} is an optionally substituted 3-7 membered saturated or partially unsaturated heterocyclic ring having 1-2 heteroatoms independently selected from nitrogen, oxygen, and sulfur. In some embodiments, R^{6A} is an optionally substituted 5-6 membered heteroaryl ring having 1-4 heteroatoms independently selected from nitrogen, oxygen, and sulfur.

In some embodiments, R^{6A} is hydrogen or optionally substituted C₁₋₆ aliphatic. In some embodiments, R^{6A} is hydrogen or C₁₋₆ aliphatic. In some embodiments, R^{6A} is hydrogen or methyl. In some embodiments, R^{6A} is C₁₋₆ aliphatic. In some embodiments, R^{6A} is methyl.

In some embodiments, R^{6A} is selected from those depicted in the compound tables below.

As defined generally above, R^{6B} is hydrogen, halogen, —CN, —C(O)R, —C(O)OR, —C(O)NR₂, —C(O)N(R)OR, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{6B} is halogen, —CN, —C(O)R, —C(O)OR, —C(O)NR₂, —C(O)N(R)OR, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{6B} is hydrogen, halogen, —CN, —C(O)R, —C(O)OR, —C(O)NR₂, or —C(O)N(R)OR. In some embodiments, R^{6B} is halogen, —CN, —C(O)R, —C(O)OR, —C(O)NR₂, or —C(O)N(R)OR. In some embodiments, R^{6B} is an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{6B} is hydrogen. In some embodiments, R^{6B} is halogen. In some embodiments, R^{6B} is —CN. In some embodiments, R^{6B} is —C(O)R. In some embodiments, R^{6B} is —C(O)OR. In some embodiments, R^{6B} is —C(O)NR₂. In some embodiments, R^{6B} is —C(O)N(R)OR. In some embodiments, R^{6B} is an optionally substituted C₁₋₆ aliphatic. In some embodiments, R^{6B} is an optionally substituted phenyl. In some embodiments, R^{6B} is an optionally substituted 3-7 membered saturated or partially unsaturated heterocyclic ring having 1-2 heteroatoms independently selected from nitrogen, oxygen, and sulfur. In some embodiments, R^{6B} is an optionally substituted 5-6 membered heteroaryl ring having 1-4 heteroatoms independently selected from nitrogen, oxygen, and sulfur.

In some embodiments, R^{6B} is hydrogen or optionally substituted C₁₋₆ aliphatic. In some embodiments, R^{6B} is hydrogen or C₁₋₆ aliphatic. In some embodiments, R^{6B} is hydrogen or methyl. In some embodiments, R^{6B} is C₁₋₆ aliphatic. In some embodiments, R^{6B} is methyl.

In some embodiments, R^{6B} is hydrogen or substituted C₁₋₆ aliphatic. In some embodiments, R^{6B} is hydrogen or C₁₋₆ aliphatic, wherein the C₁₋₆ aliphatic is substituted with —N(C₁₋₆ aliphatic)₂ or a 3-12-membered saturated monocyclic ring having 1-2 heteroatoms independently selected from nitrogen and oxygen. In some embodiments, R^{6B} is hydrogen or —CH₂—N(C₁₋₃ aliphatic)₂. In some embodiments, R^{6B} is substituted C₁₋₆ aliphatic. In some embodiments, R^{6B} is C₁₋₆ aliphatic, wherein the C₁₋₆ aliphatic is substituted with —N(C₁₋₆ aliphatic)₂ or a 3-12-membered saturated monocyclic ring having 1-2 heteroatoms independently selected from nitrogen and oxygen. In some embodiments, R^{6B} is —CH₂—N(C₁₋₃ aliphatic)₂.

In some embodiments, R^{6B} is selected from those depicted in the compound tables below.

As defined generally above, R^{6C} is hydrogen, halogen, —CN, —C(O)R, —C(O)OR, —C(O)NR₂, —C(O)N(R)OR, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{6C} is halogen, —CN, —C(O)R, —C(O)OR, —C(O)NR₂, —C(O)N(R)OR, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{6C} is hydrogen, halogen, —CN, —C(O)R, —C(O)OR, —C(O)NR₂, or —C(O)N(R)OR. In some embodiments, R^{6C} is halogen, —CN, —C(O)R, —C(O)OR, —C(O)NR₂, or —C(O)N(R)OR. In some embodiments, R^{6C} is an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{6C} is hydrogen. In some embodiments, R^{6C} is halogen. In some embodiments, R^{6C} is —CN. In some embodiments, R^{6C} is —C(O)R. In some embodiments, R^{6C} is —C(O)OR. In some embodiments, R^{6C} is —C(O)NR₂. In some embodiments, R^{6C} is —C(O)N(R)OR. In some embodiments, R^{6C} is an optionally substituted C₁₋₆ aliphatic. In some embodiments, R^{6C} is an optionally substituted phenyl. In some embodiments, R^{6C} is an optionally substituted 3-7 membered saturated or partially unsaturated heterocyclic ring having 1-2 heteroatoms independently selected from nitrogen, oxygen, and sulfur. In some embodiments, R^{6C} is an optionally substituted 5-6 membered heteroaryl ring having 1-4 heteroatoms independently selected from nitrogen, oxygen, and sulfur.

In some embodiments, R^{6C} is hydrogen or optionally substituted C₁₋₆ aliphatic. In some embodiments, R^{6C} is hydrogen or C₁₋₆ aliphatic. In some embodiments, R^{6C} is hydrogen or methyl. In some embodiments, R^{6C} is C₁₋₆ aliphatic. In some embodiments, R^{6C} is methyl.

In some embodiments, R^{6C} is selected from those depicted in the compound tables below.

As defined generally above, R^{6D} is halogen or —OS(O)₂R.

In some embodiments, R^{6D} is halogen. In some embodiments, R^{6D} is chloro. In some embodiments, R^{6D} is —OS(O)₂R.

In some embodiments, R^{6D} is selected from those depicted in the compound tables below.

As defined generally above, each instance of R⁵ is independently hydrogen or R^{L}. In some embodiments, R⁵ is hydrogen. In some embodiments, each instance of R⁵ is independently R^{L}.

In some embodiments, each instance of R⁵ is independently hydrogen or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur. In some embodiments, each instance of R⁵ is independently hydrogen or optionally substituted C₁₋₆ aliphatic. In some embodiments, each instance of R⁵ is independently hydrogen or C₁₋₆ aliphatic. In some embodiments, each instance of R⁵ is independently hydrogen or C₁₋₆ alkyl. In some embodiments, R⁵ is hydrogen or methyl.

In some embodiments, each instance of R⁵ is independently an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur. In some embodiments, each instance of R⁵ is independently optionally substituted C₁₋₆ aliphatic. In some embodiments, each instance of R⁵ is independently C₁₋₆ aliphatic. In some embodiments, each instance of R⁵ is independently C₁₋₆ alkyl. In some embodiments, R⁵ is methyl.

In some embodiments, R⁵ is selected from those depicted in the compound tables below.

As defined generally above, each instance of R⁷ is independently hydrogen or R^{L}. In some embodiments, R⁷ is hydrogen. In some embodiments, each instance of R⁷ is independently R^{L}.

In some embodiments, each instance of R⁷ is independently hydrogen, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, or an optionally substituted C₁₋₆ aliphatic. In some embodiments, each instance of R⁷ is independently hydrogen, halogen, —CN, —OR, —SR, —C(O)R, —C(O)OR, or an optionally substituted C₁₋₆ aliphatic. In some embodiments, each instance of R⁷ is independently hydrogen, halogen, —OR, or an optionally substituted C₁₋₆ aliphatic. In some embodiments, each instance of R⁷ is independently hydrogen, —OH, —CH₃, —F, or —OMe.

In some embodiments, each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, or an optionally substituted C₁₋₆ aliphatic. In some embodiments, each instance of R⁷ is independently halogen, —CN, —OR, —SR, —C(O)R, —C(O)OR, or an optionally substituted C₁₋₆ aliphatic. In some embodiments, each instance of R⁷ is independently halogen, —OR, or an optionally substituted C₁₋₆ aliphatic. In some embodiments, each instance of R⁷ is independently fluoro, —OH, —OMe, or methyl.

In some embodiments, R⁷ is —OH. In some embodiments, R⁷ is C₁₋₆ alkyl. In some embodiments, R⁷ is methyl, ethyl, i-propyl, n-propyl, i-butyl, t-butyl, or n-butyl. In some embodiments, R⁷ is methyl. In some embodiments, R⁷ is halogen. In some embodiments, R⁷ is —F or —Cl. In some embodiments, R⁷ is —OR. In some embodiments, R⁷ is —OMe.

In some embodiments, each instance of R⁷ is different. In some embodiments, two instances of R⁷ are the same.

In some embodiments, R⁷ is selected from those depicted in the compound tables below.

As defined generally above, each instance of R⁸ is independently hydrogen or R^{L}. In some embodiments, R⁸ is hydrogen. In some embodiments, each instance of R⁸ is independently R^{L}.

In some embodiments, each instance of R⁸ is independently hydrogen, oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, or an optionally substituted C₁₋₆ aliphatic. In some embodiments, each instance of R⁸ is independently hydrogen, halogen, —CN, —OR, —SR, —C(O)R, —C(O)OR, or an optionally substituted C₁₋₆ aliphatic. In some embodiments, each instance of R⁸ is independently hydrogen, halogen, —OR, or an optionally substituted C₁₋₆ aliphatic. In some embodiments, each instance of R⁸ is independently hydrogen, —OH, —CH₃, —F, —CH₂OH, —CH₂OMe, or —OMe.

In some embodiments, each instance of R⁸ is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, or an optionally substituted C₁₋₆ aliphatic. In some embodiments, each instance of R⁸ is independently halogen, —CN, —OR, —SR, —C(O)R, —C(O)OR, or an optionally substituted C₁₋₆ aliphatic. In some embodiments, each instance of R⁸ is independently halogen, —OR, or an optionally substituted C₁₋₆ aliphatic. In some embodiments, each instance of R⁸ is independently —OH, —CH₃, —F, —CH₂OH, —CH₂OMe, or —OMe.

In some embodiments, R⁸ is —OH. In some embodiments, R⁸ is C₁₋₆ alkyl. In some embodiments, R⁸ is methyl, ethyl, i-propyl, n-propyl, i-butyl, t-butyl, or n-butyl. In some embodiments, R⁸ is methyl. In some embodiments, R⁸ is —CH₂OH. In some embodiments, R⁸ is —CH₂OMe.

In some embodiments, each instance of R⁸ is different. In some embodiments, two instances of R⁸ are the same.

In some embodiments, R⁸ is selected from those depicted in the compound tables below.

As defined generally above, each instance of R¹⁰ is independently hydrogen or R^{L}. In some embodiments, R¹⁰ is hydrogen. In some embodiments, each instance of R¹⁰ is independently R^{L}.

In some embodiments, each instance of R¹⁰ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, or an optionally substituted C₁₋₆ aliphatic. In some embodiments, each instance of R¹⁰ is independently halogen, —CN, —OR, —SR, —S(O)₂R, or an optionally substituted C₁₋₆ aliphatic. In some embodiments, each instance of R¹⁰ is independently halogen, —CN, —O(C₁₋₆ aliphatic), or a C₁₋₆ aliphatic optionally substituted with —OH and/or halogen. In some embodiments, each instance of R¹⁰ is independently fluoro, chloro, bromo, —CN, —OH, —OMe, —OEt, —OiPr, —OcPr, —OCF₃, —OCHF₂, —OCH₂F, -Me, -Et, -iPr, -cPr, —CF₃, —CHF₂, —CH₂F, or —CH₂OH.

In some embodiments, R¹⁰ is selected from those depicted in the compound tables below.

As defined generally above, each instance of R^{L} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{L} is oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{L} is an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{L} is oxo. In some embodiments, R^{L} is halogen. In some embodiments, R^{L} is —CN. In some embodiments, R^{L} is —NO₂. In some embodiments, R^{L} is —OR.

In some embodiments, R^{L} is —SR. In some embodiments, R^{L} is —NR₂. In some embodiments, R^{L} is —S(O)₂R. In some embodiments, R^{L} is —S(O)₂NR₂. In some embodiments, R^{L} is —S(O)R. In some embodiments, R^{L} is —S(O)NR₂. In some embodiments, R^{L} is —C(O)R. In some embodiments, R^{L} is —C(O)OR. In some embodiments, R^{L} is —C(O)NR₂. In some embodiments, R^{L} is —C(O)N(R)OR. In some embodiments, R^{L} is —OC(O)R. In some embodiments, R^{L} is —OC(O)NR₂. In some embodiments, R^{L} is —N(R)C(O)OR. In some embodiments, R^{L} is —N(R)C(O)R. In some embodiments, R^{L} is —N(R)C(O)NR₂. In some embodiments, R^{L} is —N(R)C(NR)NR₂. In some embodiments, R^{L} is —N(R)S(O)₂NR₂. In some embodiments, R^{L} is —N(R)S(O)₂R.

In some embodiments, R^{L} is selected from those depicted in the compound tables below.

As defined generally above, R^{Y} is hydrogen or R^{L}.

In some embodiments, R^{Y} is hydrogen. In some embodiments, R^{Y} is R^{L}.

In some embodiments, R^{Y} is hydrogen, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, or an optionally substituted C₁₋₆ aliphatic. In some embodiments, R^{Y} is hydrogen, halogen, —CN, —OR, —SR, —C(O)R, —C(O)OR, or an optionally substituted C₁₋₆ aliphatic. In some embodiments, R^{Y} is hydrogen, halogen, —OR, or an optionally substituted C₁₋₆ aliphatic. In some embodiments, R^{Y} is hydrogen, fluoro, or methyl.

In some embodiments, R^{Y} is selected from those depicted in the compound tables below.

As defined generally above, R⁹ is —R^{Y}—X—R²; or a ring selected from the group consisting of phenyl; a 5-6 membered monocyclic heteroaryl ring having 1-4 heteroatoms independently selected from nitrogen, oxygen, and sulfur; and an 8-10 membered bicyclic ring having 1-4 heteroatoms independently selected from nitrogen, oxygen, and sulfur; wherein said ring is substituted by p instances of R¹⁰.

In some embodiments, R⁹ is —R^{Y}—X—R². In some embodiments, R⁹ is a ring selected from the group consisting of phenyl; a 5-6 membered monocyclic heteroaryl ring having 1-4 heteroatoms independently selected from nitrogen, oxygen, and sulfur; and an 8-10 membered bicyclic ring having 1-4 heteroatoms independently selected from nitrogen, oxygen, and sulfur; wherein said ring is substituted by p instances of R¹⁰.

In some embodiments, R⁹ is phenyl; wherein said phenyl is substituted by p instances of R¹⁰. In some embodiments, R⁹ is a 5-6 membered monocyclic heteroaryl ring having 1-4 heteroatoms independently selected from nitrogen, oxygen, and sulfur; wherein said ring is substituted by p instances of R¹⁰. In some embodiments, R⁹ is an 8-10 membered bicyclic ring having 1-4 heteroatoms independently selected from nitrogen, oxygen, and sulfur; wherein said ring is substituted by p instances of R¹⁰.

In some embodiments, R⁹ is phenyl or a 5-6 membered monocyclic heteroaryl ring having 1-4 heteroatoms independently selected from nitrogen, oxygen, and sulfur; each of which is substituted by p instances of R¹⁰. In some embodiments, R⁹ is a ring selected from a 5-6 membered monocyclic heteroaryl ring having 1-4 heteroatoms independently selected from nitrogen, oxygen, and sulfur; and an 8-10 membered bicyclic ring having 1-4 heteroatoms independently selected from nitrogen, oxygen, and sulfur; wherein said ring is substituted by p instances of R¹⁰.

In some embodiments, R⁹ is a 6-membered monocyclic heteroaryl ring having 1-4 heteroatoms independently selected from nitrogen, oxygen, and sulfur; wherein said ring is substituted by p instances of R¹⁰. In some embodiments, R⁹ is a 6-membered monocyclic heteroaryl ring having 1-2 nitrogen atoms; wherein said ring is substituted by p instances of R¹⁰. In some embodiments, R⁹ is 2-pyridinyl substituted by p instances of R¹⁰.

In some embodiments, R⁹ is a 5-membered monocyclic heteroaryl ring having 1-4 heteroatoms independently selected from nitrogen, oxygen, and sulfur; wherein said ring is substituted by p instances of R¹⁰. In some embodiments, R⁹ is a 5-membered monocyclic heteroaryl ring having 1-2 heteroatoms independently selected from nitrogen, oxygen, and sulfur; wherein said ring is substituted by p instances of R¹⁰.

In some embodiments, R⁹ is an 8-10 membered bicyclic ring having 1-2 heteroatoms independently selected from nitrogen and oxygen; wherein said ring is substituted by p instances of R¹⁰. In some embodiments, R⁹ is

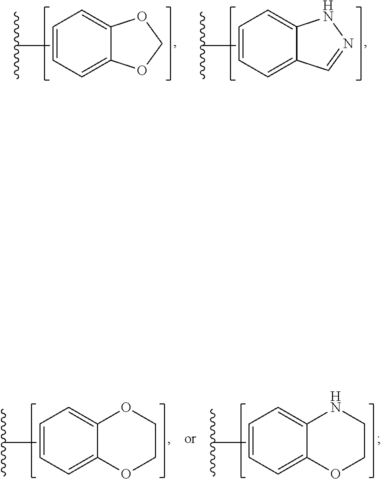

each of which is substituted by p instances of R¹⁰. In some embodiments, R⁹ is

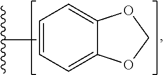

which is substituted by p instances of R¹⁰. In some embodiments, R⁹ is

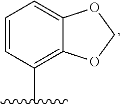 [SMILES: *c1cccc2c1OCO2]

which is substituted by p instances of R¹⁰. In some embodiments, R⁹ is

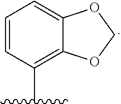 [SMILES: *c1cccc2c1OCO2]

In some embodiments, R⁹ is selected from those depicted in the compound tables below.

As defined generally above, R¹ is selected from arylene and heteroarylene; each of which is optionally substituted.

In some embodiments, R¹ is optionally substituted arylene. In some embodiments, R¹ is optionally substituted heteroarylene.

In some embodiments, R¹ is arylene. In some embodiments, R¹ is substituted arylene. In some embodiments, R¹ is optionally substituted phenylene. In some embodiments, R¹ is phenylene. In some embodiments, R¹ is substituted phenylene.

In some embodiments, R¹ is heteroarylene. In some embodiments, R¹ is substituted heteroarylene. In some embodiments, R¹ is optionally substituted monocyclic heteroarylene. In some embodiments, R¹ is monocyclic heteroarylene. In some embodiments, R¹ is substituted monocyclic heteroarylene. In some embodiments, R¹ is optionally substituted 6-membered heteroarylene. In some embodiments, R¹ is 6-membered heteroarylene. In some embodiments, R¹ is substituted 6-membered heteroarylene. In some embodiments, R¹ is optionally substituted pyridinylene. In some embodiments, R¹ is pyridinylene. In some embodiments, R¹ is substituted pyridinylene.

In some embodiments, R¹ is phenylene optionally substituted with p instances of R¹⁰. In some embodiments, R¹ is phenylene optionally substituted with 1-2 substituents independently selected from halogen, —CN, —O(C₁₋₆ aliphatic), —O(C₁₋₆ haloaliphatic), C₁₋₆ aliphatic, and C₁₋₆ haloaliphatic. In some embodiments, R¹ is phenylene optionally substituted with fluoro, chloro, —CN, —OMe, —OEt, —OCF₃, —OCHF₂, —OCH₂F, or —CH₃. In some embodiments, R¹ is phenylene optionally substituted with 1-2 substituents independently selected from halogen, —O(C₁₋₆ aliphatic), —O(C₁₋₆ haloaliphatic), C₁₋₆ aliphatic, and C₁₋₆ haloaliphatic. In some embodiments, R¹ is phenylene optionally substituted with fluoro, chloro, —OMe, —OEt, —OCF₃, —OCHF₂, —OCH₂F, or —CH₃.

In some embodiments, R¹ is phenylene substituted with p instances of R¹⁰. In some embodiments, R¹ is phenylene substituted with 1-2 substituents independently selected from halogen, —CN, —O(C₁₋₆ aliphatic), —O(C₁₋₆ haloaliphatic), C₁₋₆ aliphatic, and C₁₋₆ haloaliphatic. In some embodiments, R¹ is phenylene substituted with fluoro, chloro, —CN, —OMe, —OEt, —OCF₃, —OCHF₂, —OCH₂F, or —CH₃.

In some embodiments, R¹ is monocyclic heteroarylene optionally substituted with p instances of R¹⁰. In some embodiments, R¹ is 6-membered heteroarylene optionally substituted with p instances of R¹⁰. In some embodiments, R¹ is pyridinylene optionally substituted with p instances of R¹⁰. In some embodiments, R¹ is pyridinylene optionally substituted with 1-2 substituents independently selected from halogen, —CN, —O(C₁₋₆ aliphatic), —O(C₁₋₆ haloaliphatic), C₁₋₆ aliphatic, and C₁₋₆ haloaliphatic. In some embodiments, R¹ is pyridinylene optionally substituted with fluoro, chloro, —CN, —OMe, —OEt, —OCF₃, —OCHF₂, —OCH₂F, or —CH₃.

In some embodiments, R¹ is monocyclic heteroarylene substituted with p instances of R¹⁰. In some embodiments, R¹ is 6-membered heteroarylene substituted with p instances of R¹⁰. In some embodiments, R¹ is pyridinylene substituted with p instances of R¹⁰. In some embodiments, R¹ is pyridinylene substituted with 1-2 substituents independently selected from halogen, —CN, —O(C₁₋₆ aliphatic), —O(C₁₋₆ haloaliphatic), C₁₋₆ aliphatic, and C₁₋₆ haloaliphatic. In some embodiments, R¹ is pyridinylene substituted with fluoro, chloro, —CN, —OMe, —OEt, —OCF₃, —OCHF₂, —OCH₂F, or —CH₃.

In some embodiments, R¹ is phenylene or monocyclic heteroarylene, each of which is optionally substituted with p instances of R¹⁰. In some embodiments, R¹ is phenylene or 6-membered heteroarylene, each of which is optionally substituted with p instances of R¹⁰. In some embodiments, R¹ is phenylene or pyridinylene, each of which is optionally substituted with p instances of R¹⁰. In some embodiments, R¹ is phenylene or pyridinylene, each of which is optionally substituted with 1-2 substituents independently selected from halogen, —CN, —O(C₁₋₆ aliphatic), —O(C₁₋₆ haloaliphatic), C₁₋₆ aliphatic, and C₁₋₆ haloaliphatic. In some embodiments, R¹ is phenylene or pyridinylene, each of which is optionally substituted with fluoro, chloro, —CN, —OMe, —OEt, —OCF₃, —OCHF₂, —OCH₂F, or —CH₃.

In some embodiments, R¹ is selected from those depicted in the compound tables below.

As defined generally above, R² is selected from aryl, heteroaryl, and cycloalkyl; each of which is optionally substituted.

In some embodiments, R² is optionally substituted aryl. In some embodiments, R² is optionally substituted heteroaryl. In some embodiments, R² is optionally substituted cycloalkyl.

In some embodiments, R² is aryl or heteroaryl; each of which is optionally substituted. In some embodiments, R² is aryl or heteroaryl. In some embodiments, R² is substituted aryl or substituted heteroaryl. In some embodiments, R² is monocyclic aryl or heteroaryl, each of which is optionally substituted with p instances of R¹⁰. In some embodiments, R² is phenyl or 6-membered heteroaryl, each of which is optionally substituted with p instances of R¹⁰. In some embodiments, R² is phenyl or 6-membered heteroaryl, each of which is optionally substituted with 1-2 substituents independently selected from halogen, —CN, —O(C₁₋₆ aliphatic), —O(C₁₋₆ haloaliphatic), C₁₋₆ aliphatic, and C₁₋₆ haloaliphatic. In some embodiments, R² is phenyl or pyridinyl, each of which is optionally substituted with —F, —Cl, —CN, or —CH₃.

In some embodiments, R² is aryl. In some embodiments, R² is substituted aryl. In some embodiments, R² is optionally substituted phenyl. In some embodiments, R² is phenyl. In some embodiments, R² is substituted phenyl.

In some embodiments, R² is heteroaryl. In some embodiments, R² is substituted heteroaryl. In some embodiments, R² is optionally substituted monocyclic heteroaryl. In some embodiments, R² is monocyclic heteroaryl. In some embodiments, R² is substituted monocyclic heteroaryl. In some embodiments, R² is optionally substituted 6-membered heteroaryl. In some embodiments, R² is 6-membered heteroaryl. In some embodiments, R² is substituted 6-membered heteroaryl. In some embodiments, R² is optionally substituted pyridinyl. In some embodiments, R² is pyridinyl. In some embodiments, R² is substituted pyridinyl.

In some embodiments, R² is cycloalkyl. In some embodiments, R² is substituted cycloalkyl. In some embodiments, R² is optionally substituted C₃₋₆ cycloalkyl. In some embodiments, R² is C₃₋₆ cycloalkyl. In some embodiments, R² is substituted C₃₋₆ cycloalkyl. In some embodiments, R² is optionally substituted cyclopropyl. In some embodiments, R² is cyclopropyl. In some embodiments, R² is substituted cyclopropyl.

In some embodiments, R² is phenyl optionally substituted with p instances of R¹⁰. In some embodiments, R² is phenyl optionally substituted with 1-2 substituents independently selected from halogen, —CN, —O(C₁₋₆ aliphatic), —O(C₁₋₆ haloaliphatic), C₁₋₆ aliphatic, and C₁₋₆ haloaliphatic. In some embodiments, R² is phenyl optionally substituted with —F, —Cl, —CN, or —CH₃.

In some embodiments, R² is phenyl substituted with p instances of R¹⁰. In some embodiments, R² is phenyl substituted with 1-2 substituents independently selected from halogen, —CN, —O(C₁₋₆ aliphatic), —O(C₁₋₆ haloaliphatic), C₁₋₆ aliphatic, and C₁₋₆ haloaliphatic. In some embodiments, R² is phenyl substituted with —F, —Cl, —CN, or —CH₃.

In some embodiments, R² is monocyclic heteroaryl optionally substituted with p instances of R¹⁰. In some embodiments, R² is 6-membered heteroaryl optionally substituted with p instances of R¹⁰. In some embodiments, R² is pyridinyl optionally substituted with p instances of R¹⁰. In some embodiments, R² is pyridinyl optionally substituted with 1-2 substituents independently selected from halogen, —CN, —O(C₁₋₆ aliphatic), —O(C₁₋₆ haloaliphatic), C₁₋₆ aliphatic, and C₁₋₆ haloaliphatic. In some embodiments, R² is pyridinyl optionally substituted with —F, —Cl, —CN, or —CH₃.

In some embodiments, R² is monocyclic heteroaryl substituted with p instances of R¹⁰. In some embodiments, R² is 6-membered heteroaryl substituted with p instances of R¹⁰. In some embodiments, R² is pyridinyl substituted with p instances of R¹⁰. In some embodiments, R² is pyridinyl substituted with 1-2 substituents independently selected from halogen, —CN, —O(C₁₋₆ aliphatic), —O(C₁₋₆ haloaliphatic), C₁₋₆ aliphatic, and C₁₋₆ haloaliphatic. In some embodiments, R² is pyridinyl substituted with —F, —Cl, —CN, or —CH₃.

In some embodiments, R² is C₃₋₆ cycloalkyl substituted with p instances of R¹⁰. In some embodiments, R² is C₃₋₆ cycloalkyl substituted with 1-2 substituents independently selected from halogen, —CN, —O(C₁₋₆ aliphatic), —O(C₁₋₆ haloaliphatic), C₁₋₆ aliphatic, and C₁₋₆ haloaliphatic. In some embodiments, R² is cyclopropyl substituted with —F, —Cl, —CN, or —CH₃.

In some embodiments, R² is selected from those depicted in the compound tables below.

As defined generally above, X is a C₁₋₂ bivalent hydrocarbon chain wherein one or more methylene units of the chain are optionally and independently replaced by —CH(R^{L})—, C₃₋₅ cycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—.

In some embodiments, X is a C₁₋₂ bivalent hydrocarbon chain. In some embodiments, X is a C₁₋₂ bivalent hydrocarbon chain wherein one or more methylene units of the chain is independently replaced by —CH(R^{L})—, C₃₋₅ cycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—.

In some embodiments, X is a C₁₋₂ bivalent hydrocarbon chain wherein one or more methylene units of the chain are optionally replaced by —CH(R^{L})—. In some embodiments, X is a C₁₋₂ bivalent hydrocarbon chain wherein one or more methylene units of the chain are optionally replaced by C₃₋₅ cycloalkylene. In some embodiments, X is a C₁₋₂ bivalent hydrocarbon chain wherein one or more methylene units of the chain are optionally replaced by —N(R)—. In some embodiments, X is a C₁₋₂ bivalent hydrocarbon chain wherein one or more methylene units of the chain are optionally replaced by —N(R)C(O)—. In some embodiments, X is a C₁₋₂ bivalent hydrocarbon chain wherein one or more methylene units of the chain are optionally replaced by —C(O)N(R)—. In some embodiments, X is a C₁₋₂ bivalent hydrocarbon chain wherein one or more methylene units of the chain are optionally replaced by —N(R)S(O)₂—. In some embodiments, X is a C₁₋₂ bivalent hydrocarbon chain wherein one or more methylene units of the chain are optionally replaced by —S(O)₂N(R)—. In some embodiments, X is a C₁₋₂ bivalent hydrocarbon chain wherein one or more methylene units of the chain are optionally replaced by —O—. In some embodiments, X is a C₁₋₂ bivalent hydrocarbon chain wherein one or more methylene units of the chain are optionally replaced by —C(O)—. In some embodiments, X is a C₁₋₂ bivalent hydrocarbon chain wherein one or more methylene units of the chain are optionally replaced by —OC(O)—. In some embodiments, X is a C₁₋₂ bivalent hydrocarbon chain wherein one or more methylene units of the chain are optionally replaced by —C(O)O—. In some embodiments, X is a C₁₋₂ bivalent hydrocarbon chain wherein one or more methylene units of the chain are optionally replaced by —S—. In some embodiments, X is a C₁₋₂ bivalent hydrocarbon chain wherein one or more methylene units of the chain are optionally replaced by —S(O)—. In some embodiments, X is a C₁₋₂ bivalent hydrocarbon chain wherein one or more methylene units of the chain are optionally replaced by —S(O)₂—.

In some embodiments, X is —O—, —N(H)—, —OCH₂—, or —C(H)(CN)—. In some embodiments, X is —O—. In some embodiments, X is —N(H)—. In some embodiments, X is —C(H)(CN)—. In some embodiments, X is —OCH₂—.

In some embodiments, X is selected from those depicted in the compound tables below.

As defined generally above, each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R is an optionally substituted C₁₋₆ aliphatic. In some embodiments, R is an optionally substituted phenyl. In some embodiments, R is an optionally substituted 3-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur.

In some embodiments, R is selected from those depicted in the compound tables below.

As defined generally above, each instance of m is independently 0-4. In some embodiments, m is 0. In some embodiments, m is 1, 2, 3, or 4. In some embodiments, m is 1. In some embodiments, m is 2. In some embodiments, m is 3. In some embodiments, m is 4.

In some embodiments, m is 1, 2, or 3. In some embodiments, m is 1 or 2. In some embodiments, m is 1 or 3. In some embodiments, m is 2 or 3. In some embodiments, m is 2 or 4. In some embodiments, m is 1, 2, or 4. In some embodiments, m is 1, 3, or 4. In some embodiments, m is 2, 3, or 4.

In some embodiments, m is selected from those depicted in the compound tables below.

As defined generally above, each instance of n is independently 0-4. In some embodiments, n is 0. In some embodiments, n is 1, 2, 3, or 4. In some embodiments, n is 1. In some embodiments, n is 2. In some embodiments, n is 3. In some embodiments, n is 4.

In some embodiments, n is 1, 2, or 3. In some embodiments, n is 1 or 2. In some embodiments, n is 1 or 3. In some embodiments, n is 2 or 3. In some embodiments, n is 2 or 4. In some embodiments, n is 1, 2, or 4. In some embodiments, n is 1, 3, or 4. In some embodiments, n is 2, 3, or 4.

In some embodiments, n is selected from those depicted in the compound tables below.

As defined generally above, p is 0-4. In some embodiments, p is 0. In some embodiments, p is 1, 2, 3, or 4. In some embodiments, p is 1. In some embodiments, p is 2. In some embodiments, p is 3. In some embodiments, p is 4.

In some embodiments, p is 1, 2, or 3. In some embodiments, p is 1 or 2. In some embodiments, p is 1 or 3. In some embodiments, p is 2 or 3. In some embodiments, p is 2 or 4. In some embodiments, p is 1, 2, or 4. In some embodiments, p is 1, 3, or 4. In some embodiments, p is 2, 3, or 4.

In some embodiments, p is selected from those depicted in the compound tables below.

In some embodiments, the present invention provides a compound of Formulas (V) or (VI) wherein R⁹ is —R¹—X—R², X is O, and R¹ is phenylene or pyridinylene; each of which is optionally substituted with p instances of R¹⁰, thereby forming a compound of Formulas (VII), (VIII), (IX), (X), (XI), or (XII):

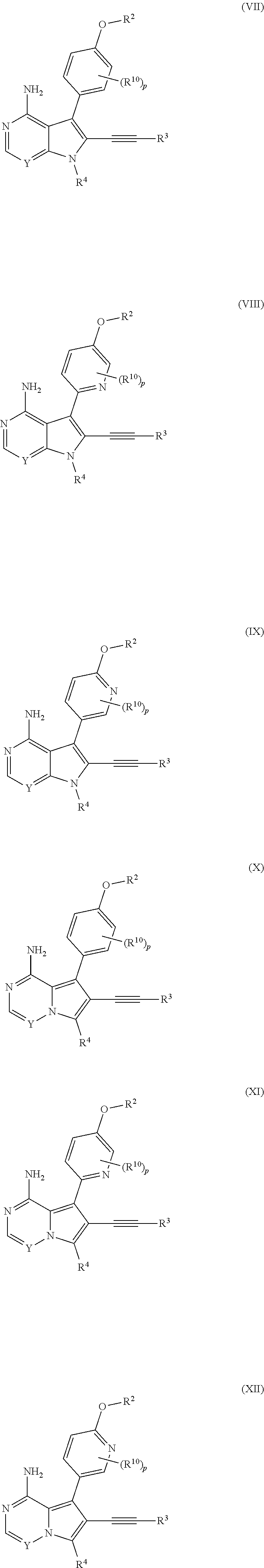

or a pharmaceutically acceptable salt thereof, wherein each of R², R³, R⁴, R¹⁰, Y, and p is as defined above and described in embodiments herein, both singly and in combination.

In some embodiments, the present invention provides a compound of Formulas (V) or (VI) wherein R⁹ is

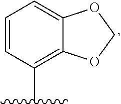 [SMILES: *c1cccc2c1OCO2]

which is substituted by p instances of R¹⁰, thereby forming a compound of Formulas (XIII) or (XIV):

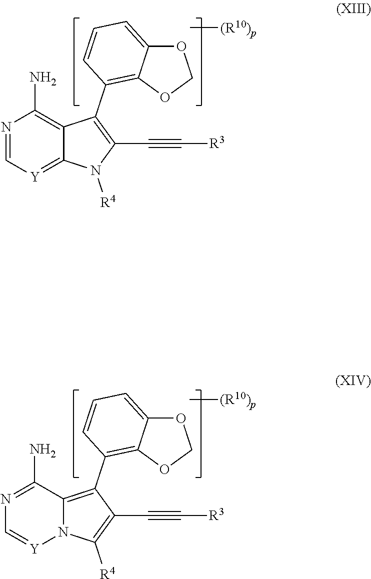

or a pharmaceutically acceptable salt thereof, wherein each of R³, R⁴, R¹⁰, Y, and p is as defined above and described in embodiments herein, both singly and in combination.

In some embodiments, the present invention provides a compound of Formulas (V) or (VI) wherein R³ is

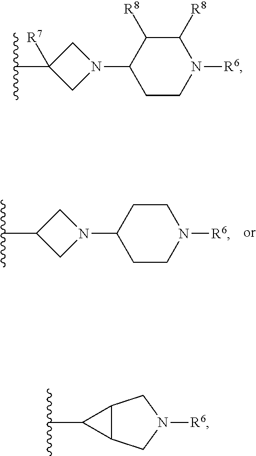

thereby forming a compound of Formulas (XV), (XVI), (XVII), (XVIII), (XIX), or (XX):

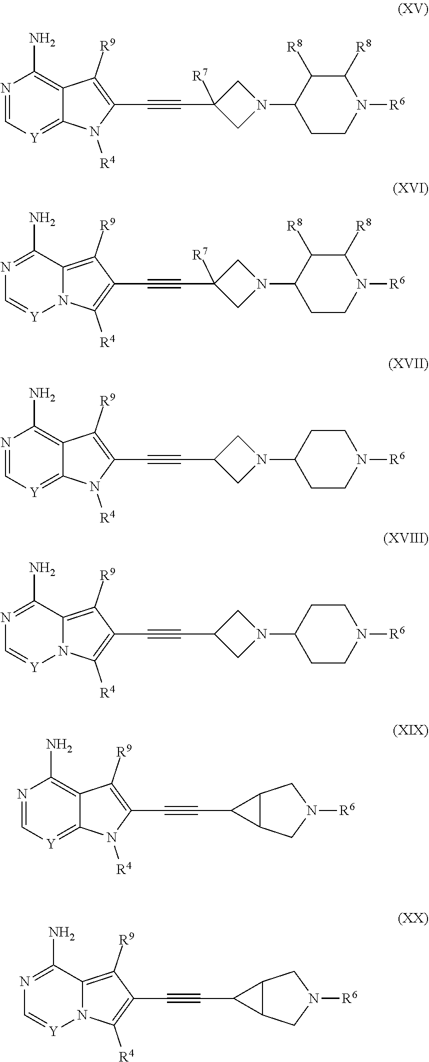

or a pharmaceutically acceptable salt thereof, wherein each of R⁴, R⁶, R⁷, R⁸, R⁹, and Y is as defined above and described in embodiments herein, both singly and in combination.

In some embodiments, the present invention provides a compound of Formulas (V), (VI), (VII), (VIII), (IX), (X), (XI), (XII), (XIII), (XIV), (XV), (XVI), (XVII), (XVIII), (XIX), or (XX) wherein Y is N, thereby forming a compound of Formulas (V-a), (VI-a), (VII-a), (VIII-a), (IX-a), (X-a), (XI-a), (XII-a), (XIII-a), (XIV-a), (XV-a), (XVI-a), (XVII-a), (XVIII-a), (XIX-a), or (XX-a):

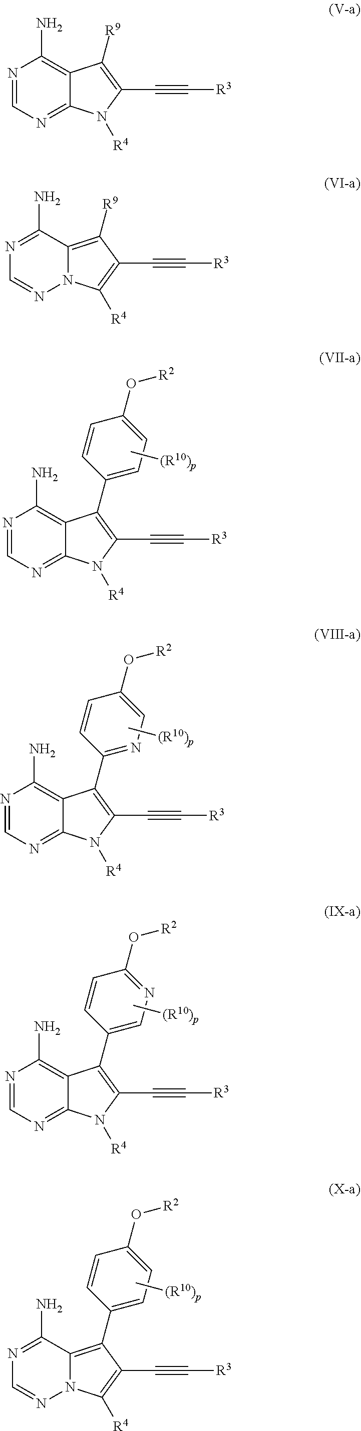

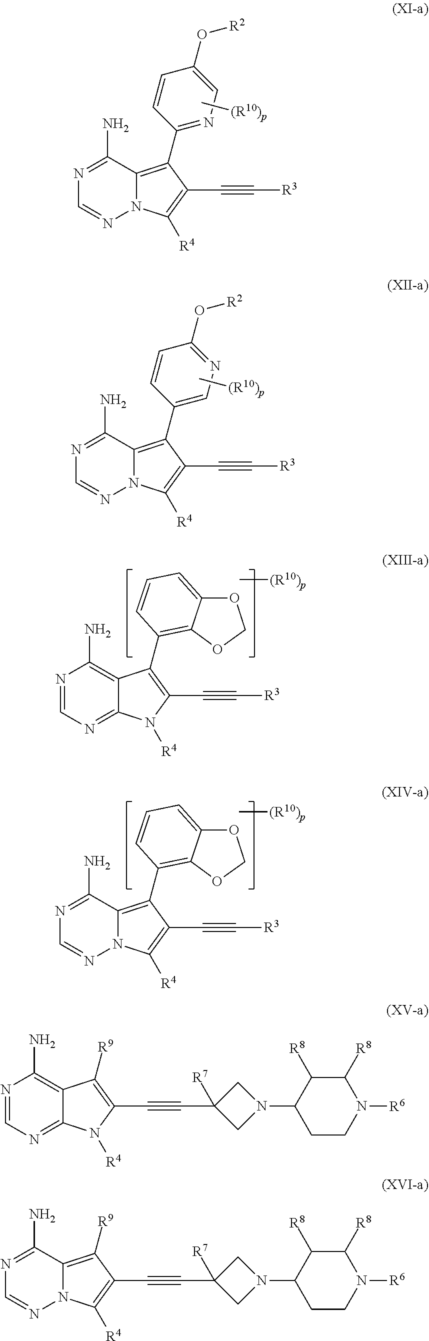

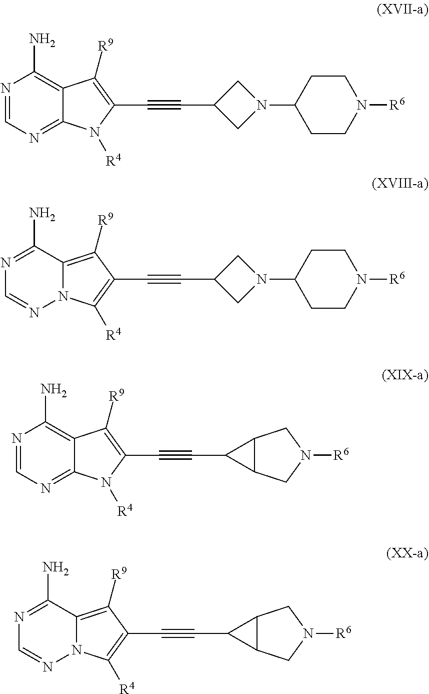

or a pharmaceutically acceptable salt thereof, wherein each of R², R³, R⁴, R⁶, R⁷, R⁸, R⁹, R¹⁰, and p is as defined above and described in embodiments herein, both singly and in combination.

In some embodiments, the present invention provides a compound of Formula (I), (II), (VII), (VIII), (IX), (X), (XI), (XII), (VII-a), (VIII-a), (IX-a), (X-a), (XI-a), or (XII-a), wherein R² is phenyl or pyridinyl, each of which is optionally substituted with —F, —Cl, —CN, or —CH₃.

In some embodiments, the present invention provides a compound of Formula (VII), (VIII), (IX), (X), (XI), (XII), (XIII), (XIV), (VII-a), (VIII-a), (IX-a), (X-a), (XI-a), (XII-a), (XIII-a), or (XIV-a) wherein p is 0. In some embodiments, the present invention provides a compound of Formula (VII), (VIII), (IX), (X), (XI), (XII), (XIII), (XIV), (VII-a), (VIII-a), (IX-a), (X-a), (XI-a), (XII-a), (XIII-a), or (XIV-a) wherein p is 1 and R¹⁰ is fluoro, chloro, —CN, —OMe, —OEt, —OCF₃, —OCHF₂, —OCH₂F, or —CH₃.

In some embodiments, the present invention provides a compound of Formula (I), (II), (XV), (XVI), (XVII), (XVIII), (XIX), (XX), (XV-a), (XVI-a), (XVII-a), (XVIII-a), (XIX-a), or (XX-a) wherein R⁶ is —C(O)C(H)CH₂.

In some embodiments, the present invention provides a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (V-a), (VI-a), (VII-a), (VIII-a), (IX-a), (X-a), (XI-a), (XII-a), (XIII-a), (XIV-a), (XV-a), (XVI-a), (XVII-a), (XVIII-a), (XIX-a), or (XX-a) wherein R⁴ is an optionally substituted C₁₋₆ aliphatic.

In some embodiments, the present invention provides a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (V-a), (VI-a), (VII-a), (VIII-a), (IX-a), (X-a), (XI-a), (XII-a), (XIII-a), (XIV-a), (XV-a), (XVI-a), (XVII-a), (XVIII-a), (XIX-a), or (XX-a) wherein R⁴ is C₁₋₃ aliphatic.

In some embodiments, the compound of the invention is selected from the group consisting of the compounds in Table 3, below:

TABLE 3
Selected Compounds

| Compound | Structure |
|---|---|
| I-1 | 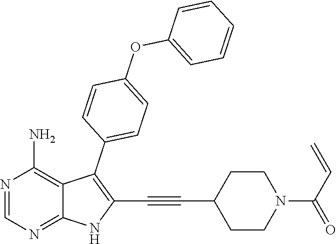 [SMILES: C=CC(=O)N1CCC(C#Cc2[nH]c3ncnc(N)c3c2-c2ccc(Oc3ccccc3)cc2)CC1] |
| I-2 | 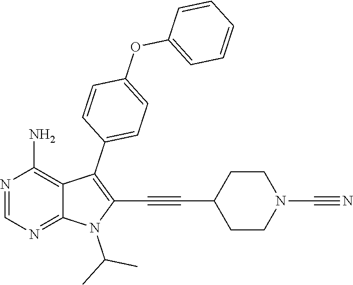 [SMILES: CC(C)n1c(C#CC2CCN(C#N)CC2)c(-c2ccc(Oc3ccccc3)cc2)c2c(N)ncnc21] |
| I-3 | 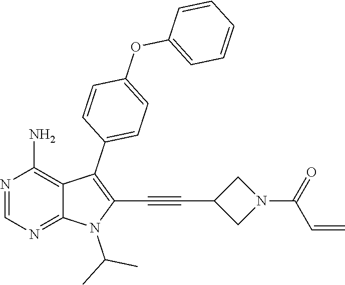 [SMILES: C=CC(=O)N1CC(C#Cc2c(-c3ccc(Oc4ccccc4)cc3)c3c(N)ncnc3n2C(C)C)C1] |
| I-4 | 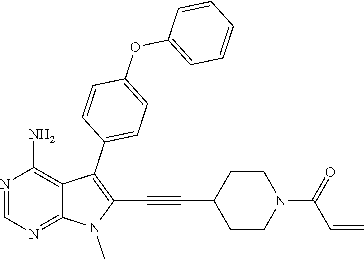 [SMILES: C=CC(=O)N1CCC(C#Cc2c(-c3ccc(Oc4ccccc4)cc3)c3c(N)ncnc3n2C)CC1] |
| I-5 | 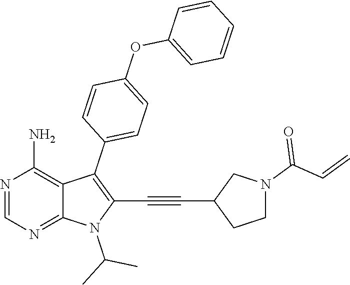 [SMILES: C=CC(=O)N1CCC(C#Cc2c(-c3ccc(Oc4ccccc4)cc3)c3c(N)ncnc3n2C(C)C)C1] |
| I-6 | 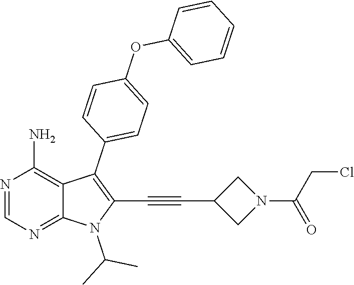 [SMILES: CC(C)n1c(C#CC2CN(C(=O)CCl)C2)c(-c2ccc(Oc3ccccc3)cc2)c2c(N)ncnc21] |
| I-7 | 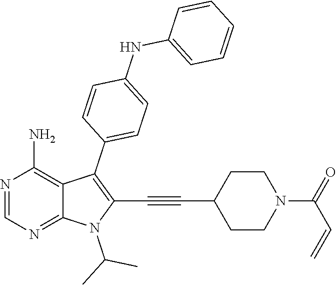 [SMILES: C=CC(=O)N1CCC(C#Cc2c(-c3ccc(Nc4ccccc4)cc3)c3c(N)ncnc3n2C(C)C)CC1] |
| I-8 | 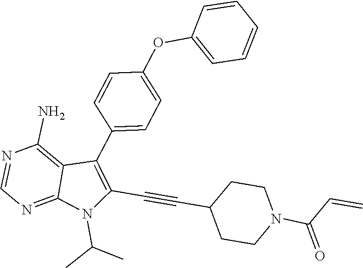 [SMILES: C=CC(=O)N1CCC(C#Cc2c(-c3ccc(Oc4ccccc4)cc3)c3c(N)ncnc3n2C(C)C)CC1] |
| I-9 | 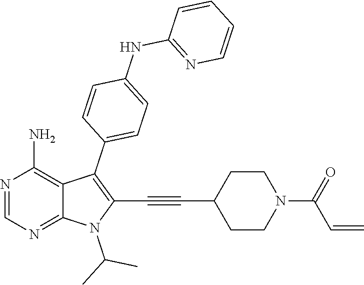 [SMILES: C=CC(=O)N1CCC(C#Cc2c(-c3ccc(Nc4ccccn4)cc3)c3c(N)ncnc3n2C(C)C)CC1] |
| I-10 | 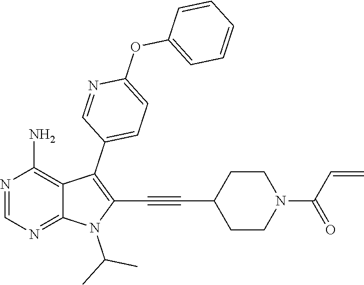 [SMILES: C=CC(=O)N1CCC(C#Cc2c(-c3ccc(Oc4ccccc4)nc3)c3c(N)ncnc3n2C(C)C)CC1] |
| I-11 | 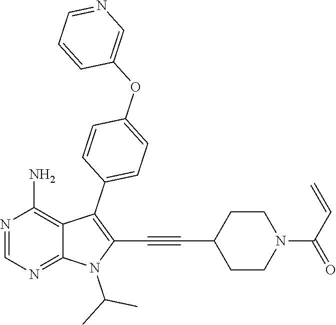 [SMILES: C=CC(=O)N1CCC(C#Cc2c(-c3ccc(Oc4cccnc4)cc3)c3c(N)ncnc3n2C(C)C)CC1] |
| I-12 | 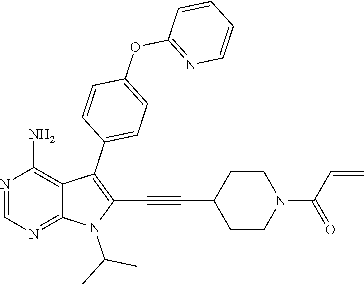 [SMILES: C=CC(=O)N1CCC(C#Cc2c(-c3ccc(Oc4ccccn4)cc3)c3c(N)ncnc3n2C(C)C)CC1] |
| I-13 | 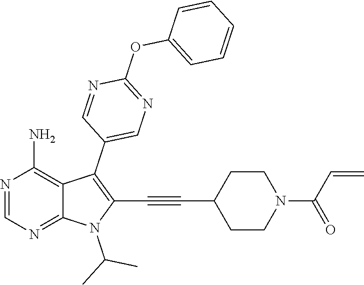 [SMILES: C=CC(=O)N1CCC(C#Cc2c(-c3cnc(Oc4ccccc4)nc3)c3c(N)ncnc3n2C(C)C)CC1] |
| I-14 | 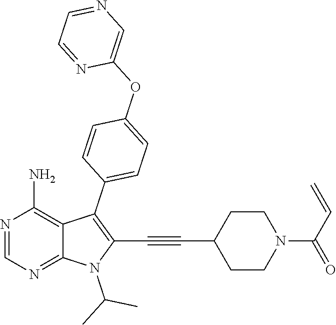 [SMILES: C=CC(=O)N1CCC(C#Cc2c(-c3ccc(Oc4cnccn4)cc3)c3c(N)ncnc3n2C(C)C)CC1] |
| I-15 | 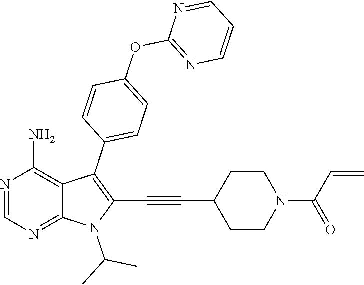 [SMILES: C=CC(=O)N1CCC(C#Cc2c(-c3ccc(Oc4ncccn4)cc3)c3c(N)ncnc3n2C(C)C)CC1] |
| I-16 | 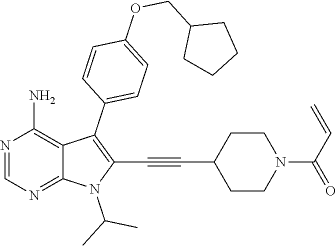 [SMILES: C=CC(=O)N1CCC(C#Cc2c(-c3ccc(OCC4CCCC4)cc3)c3c(N)ncnc3n2C(C)C)CC1] |
| I-17 | 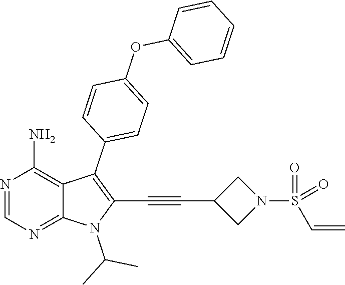 [SMILES: C=CS(=O)(=O)N1CC(C#Cc2c(-c3ccc(Oc4ccccc4)cc3)c3c(N)ncnc3n2C(C)C)C1] |
| I-18 | 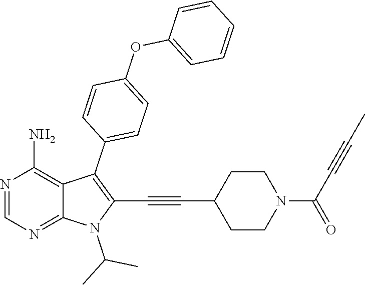 [SMILES: CC#CC(=O)N1CCC(C#Cc2c(-c3ccc(Oc4ccccc4)cc3)c3c(N)ncnc3n2C(C)C)CC1] |
| I-19 | 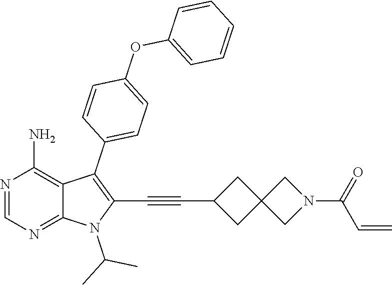 [SMILES: C=CC(=O)N1CC2(CC(C#Cc3c(-c4ccc(Oc5ccccc5)cc4)c4c(N)ncnc4n3C(C)C)C2)C1] |
| I-20 | 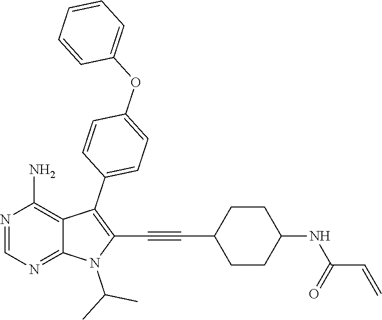 [SMILES: C=CC(=O)NC1CCC(C#Cc2c(-c3ccc(Oc4ccccc4)cc3)c3c(N)ncnc3n2C(C)C)CC1] |
| I-21 | 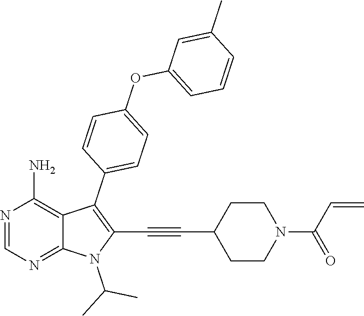 [SMILES: C=CC(=O)N1CCC(C#Cc2c(-c3ccc(Oc4cccc(C)c4)cc3)c3c(N)ncnc3n2C(C)C)CC1] |
| I-22 | 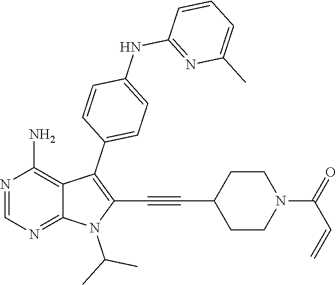 [SMILES: C=CC(=O)N1CCC(C#Cc2c(-c3ccc(Nc4cccc(C)n4)cc3)c3c(N)ncnc3n2C(C)C)CC1] |
| I-23 | 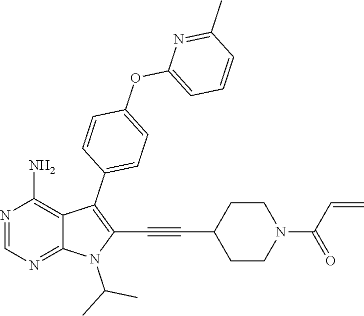 [SMILES: C=CC(=O)N1CCC(C#Cc2c(-c3ccc(Oc4cccc(C)n4)cc3)c3c(N)ncnc3n2C(C)C)CC1] |
| I-24 | 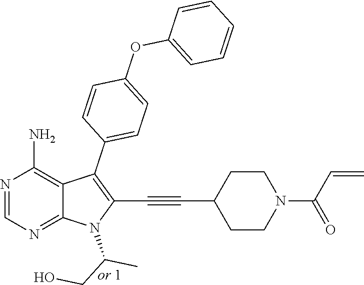 [SMILES: C=CC(=O)N1CCC(C#Cc2c(-c3ccc(Oc4ccccc4)cc3)c3c(N)ncnc3n2[C@H](C)CO)CC1] |
| I-25 | 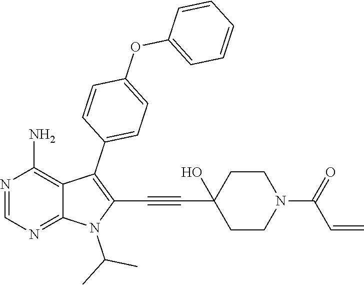 [SMILES: C=CC(=O)N1CCC(O)(C#Cc2c(-c3ccc(Oc4ccccc4)cc3)c3c(N)ncnc3n2C(C)C)CC1] |
| I-26 | 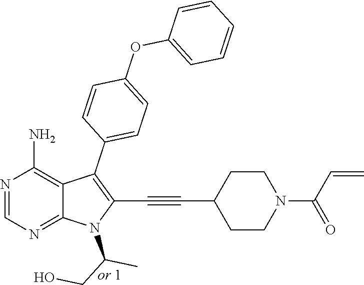 [SMILES: C=CC(=O)N1CCC(C#Cc2c(-c3ccc(Oc4ccccc4)cc3)c3c(N)ncnc3n2[C@@H](C)CO)CC1] |
| I-27 | 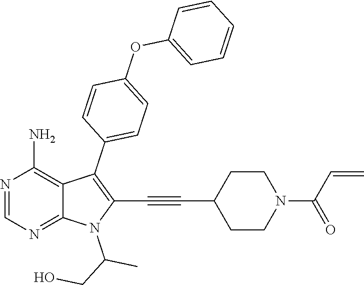 [SMILES: C=CC(=O)N1CCC(C#Cc2c(-c3ccc(Oc4ccccc4)cc3)c3c(N)ncnc3n2C(C)CO)CC1] |
| I-28 | 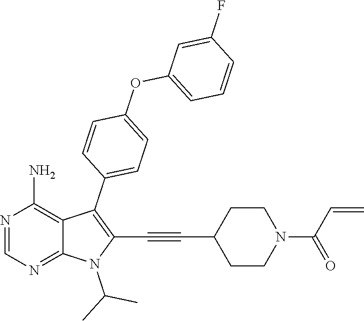 [SMILES: C=CC(=O)N1CCC(C#Cc2c(-c3ccc(Oc4cccc(F)c4)cc3)c3c(N)ncnc3n2C(C)C)CC1] |
| I-29 | 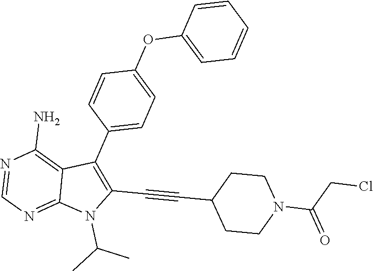 [SMILES: CC(C)n1c(C#CC2CCN(C(=O)CCl)CC2)c(-c2ccc(Oc3ccccc3)cc2)c2c(N)ncnc21] |
| I-30 | 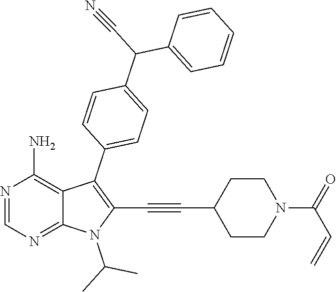 [SMILES: C=CC(=O)N1CCC(C#Cc2c(-c3ccc(C(C#N)c4ccccc4)cc3)c3c(N)ncnc3n2C(C)C)CC1] |
| I-31 | 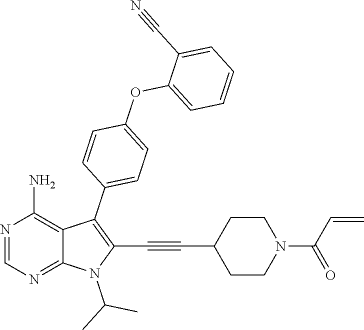 [SMILES: C=CC(=O)N1CCC(C#Cc2c(-c3ccc(Oc4ccccc4C#N)cc3)c3c(N)ncnc3n2C(C)C)CC1] |
| I-32 | 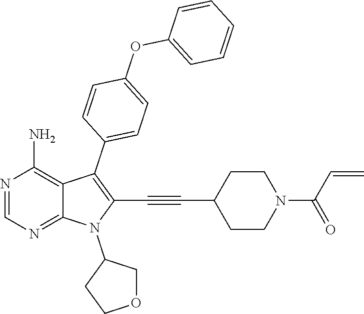 [SMILES: C=CC(=O)N1CCC(C#Cc2c(-c3ccc(Oc4ccccc4)cc3)c3c(N)ncnc3n2C2CCOC2)CC1] |
| I-33 | 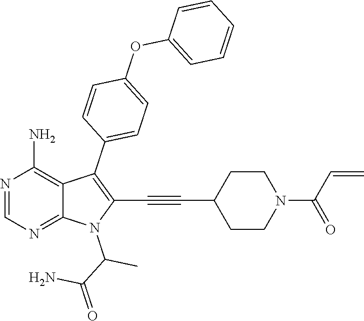 [SMILES: C=CC(=O)N1CCC(C#Cc2c(-c3ccc(Oc4ccccc4)cc3)c3c(N)ncnc3n2C(C)C(N)=O)CC1] |
| I-34 | 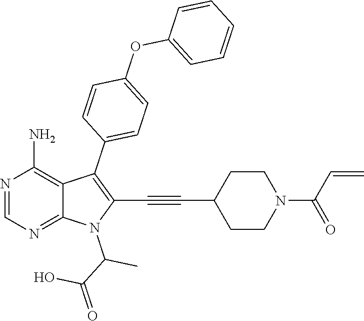 [SMILES: C=CC(=O)N1CCC(C#Cc2c(-c3ccc(Oc4ccccc4)cc3)c3c(N)ncnc3n2C(C)C(=O)O)CC1] |
| I-35 | 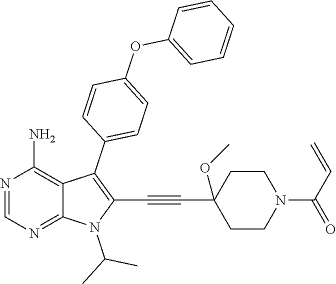 [SMILES: C=CC(=O)N1CCC(C#Cc2c(-c3ccc(Oc4ccccc4)cc3)c3c(N)ncnc3n2C(C)C)(OC)CC1] |
| I-36 | 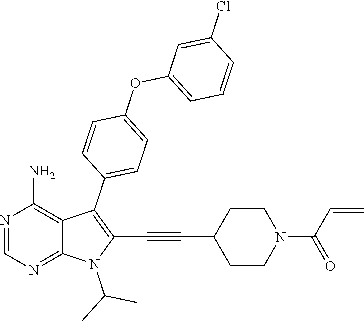 [SMILES: C=CC(=O)N1CCC(C#Cc2c(-c3ccc(Oc4cccc(Cl)c4)cc3)c3c(N)ncnc3n2C(C)C)CC1] |
| I-37 | 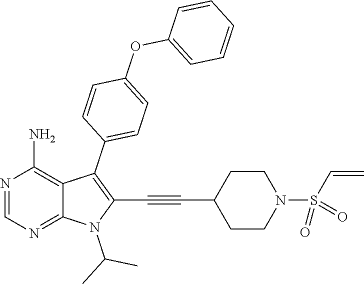 [SMILES: C=CS(=O)(=O)N1CCC(C#Cc2c(-c3ccc(Oc4ccccc4)cc3)c3c(N)ncnc3n2C(C)C)CC1] |
| I-38 | 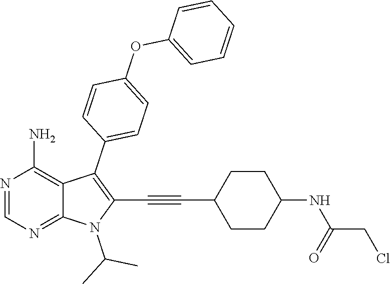 [SMILES: CC(C)n1c(C#CC2CCC(NC(=O)CCl)CC2)c(-c2ccc(Oc3ccccc3)cc2)c2c(N)ncnc21] |
| I-39 | 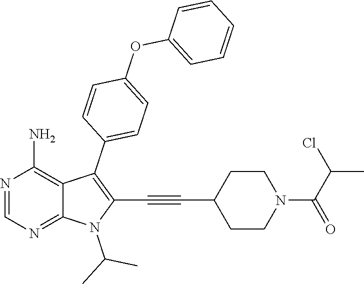 [SMILES: CC(Cl)C(=O)N1CCC(C#Cc2c(-c3ccc(Oc4ccccc4)cc3)c3c(N)ncnc3n2C(C)C)CC1] |
| I-40 | 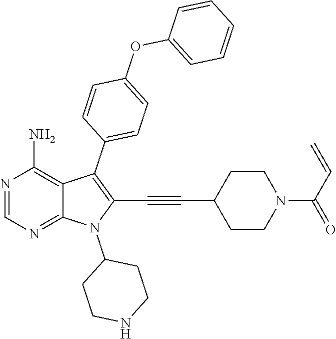 [SMILES: C=CC(=O)N1CCC(C#Cc2c(-c3ccc(Oc4ccccc4)cc3)c3c(N)ncnc3n2C2CCNCC2)CC1] |
| I-41 | 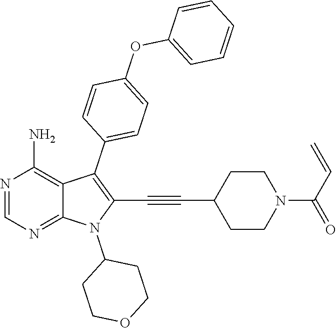 [SMILES: C=CC(=O)N1CCC(C#Cc2c(-c3ccc(Oc4ccccc4)cc3)c3c(N)ncnc3n2C2CCOCC2)CC1] |
| I-42 | 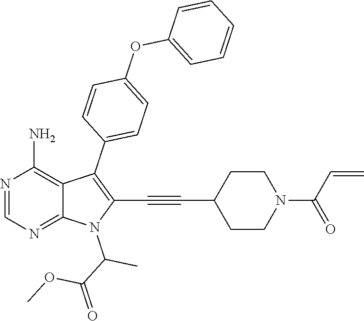 [SMILES: C=CC(=O)N1CCC(C#Cc2c(-c3ccc(Oc4ccccc4)cc3)c3c(N)ncnc3n2C(C)C(=O)OC)CC1] |
| I-43 | 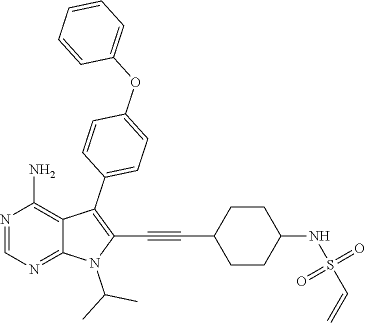 [SMILES: C=CS(=O)(=O)NC1CCC(C#Cc2c(-c3ccc(Oc4ccccc4)cc3)c3c(N)ncnc3n2C(C)C)CC1] |
| I-44 | 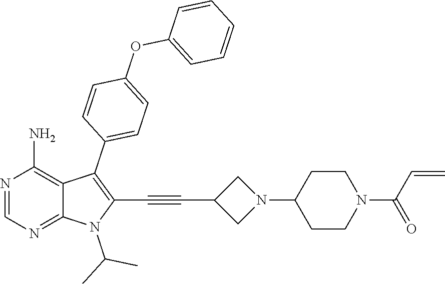 [SMILES: C=CC(=O)N1CCC(N2CC(C#Cc3c(-c4ccc(Oc5ccccc5)cc4)c4c(N)ncnc4n3C(C)C)C2)CC1] |
| I-45 | 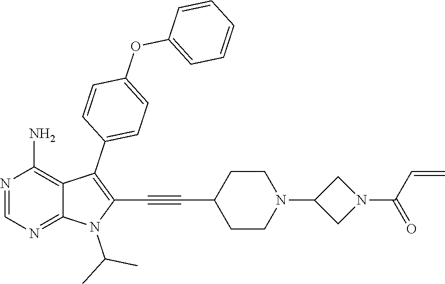 [SMILES: C=CC(=O)N1CC(N2CCC(C#Cc3c(-c4ccc(Oc5ccccc5)cc4)c4c(N)ncnc4n3C(C)C)CC2)C1] |
| I-46 | 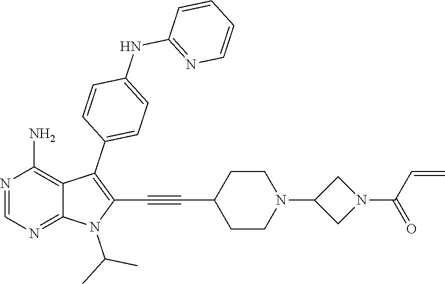 [SMILES: C=CC(=O)N1CC(N2CCC(C#Cc3c(-c4ccc(Nc5ccccn5)cc4)c4c(N)ncnc4n3C(C)C)CC2)C1] |
| I-47 | 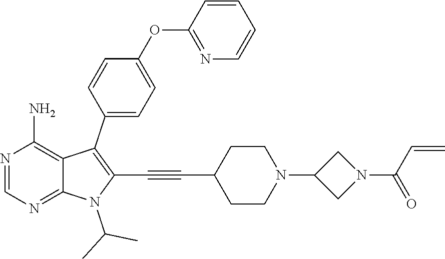 [SMILES: C=CC(=O)N1CC(N2CCC(C#Cc3c(-c4ccc(Oc5ccccn5)cc4)c4c(N)ncnc4n3C(C)C)CC2)C1] |
| I-48 | 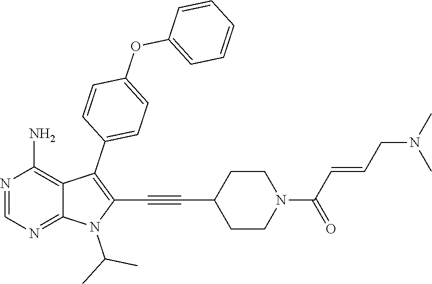 [SMILES: CC(C)n1c(C#CC2CCN(C(=O)/C=C/CN(C)C)CC2)c(-c2ccc(Oc3ccccc3)cc2)c2c(N)ncnc21] |
| I-49 | 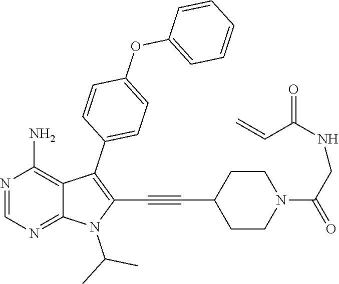 [SMILES: C=CC(=O)NCC(=O)N1CCC(C#Cc2c(-c3ccc(Oc4ccccc4)cc3)c3c(N)ncnc3n2C(C)C)CC1] |
| I-50 | 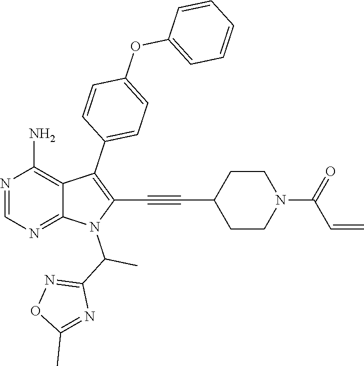 [SMILES: C=CC(=O)N1CCC(C#Cc2c(-c3ccc(Oc4ccccc4)cc3)c3c(N)ncnc3n2C(C)c2noc(C)n2)CC1] |
| I-51 | 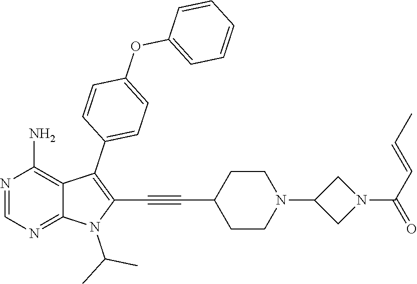 [SMILES: C/C=C/C(=O)N1CC(N2CCC(C#Cc3c(-c4ccc(Oc5ccccc5)cc4)c4c(N)ncnc4n3C(C)C)CC2)C1] |
| I-52 | 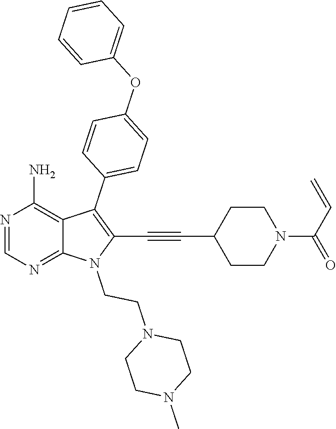 [SMILES: C=CC(=O)N1CCC(C#Cc2c(-c3ccc(Oc4ccccc4)cc3)c3c(N)ncnc3n2CCN2CCN(C)CC2)CC1] |
| I-53 | 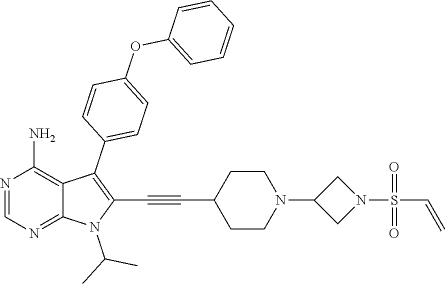 [SMILES: C=CS(=O)(=O)N1CC(N2CCC(C#Cc3c(-c4ccc(Oc5ccccc5)cc4)c4c(N)ncnc4n3C(C)C)CC2)C1] |
| I-54 | 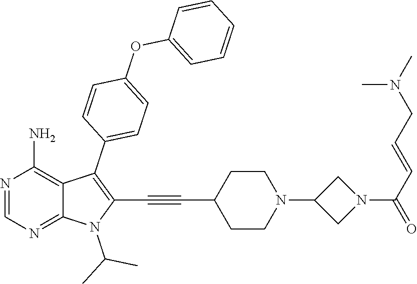 [SMILES: CC(C)n1c(C#CC2CCN(C3CN(C(=O)/C=C/CN(C)C)C3)CC2)c(-c2ccc(Oc3ccccc3)cc2)c2c(N)ncnc21] |

In some embodiments, the compound of the invention is selected from the group consisting of the compounds in Table 3, above, or a pharmaceutically acceptable salt thereof. In some embodiments, the compound of the invention is selected from the group consisting of a pharmaceutically acceptable salt of the compounds in Table 3, above.

In some embodiments, the compound of the invention is selected from the group consisting of the compounds in Table 4, below:

TABLE 4
Selected Compounds

| Com- | |
|---|---|
| pound | Structure |
| I-55 | 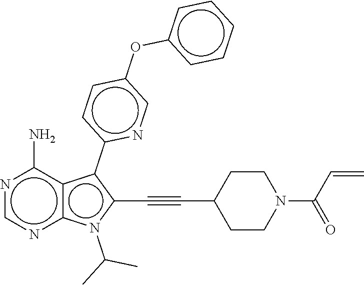 |
| I-56 | 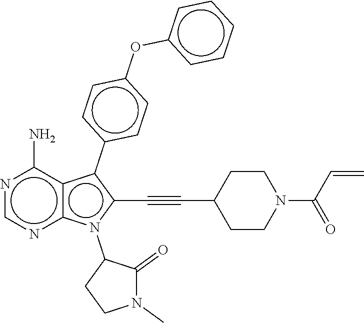 |
| I-57 | 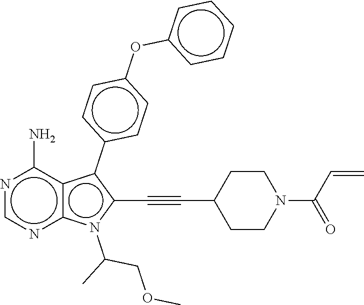 |
| I-58 | 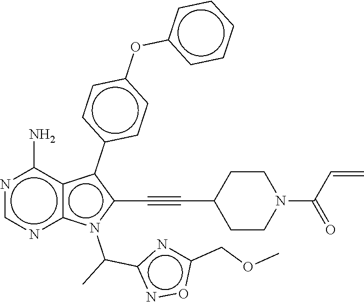 |
| I-59 | 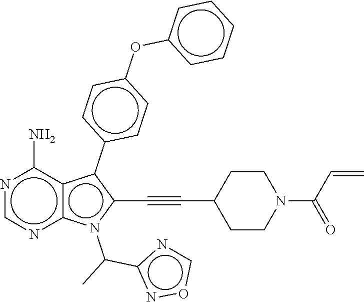 |
| I-60 | 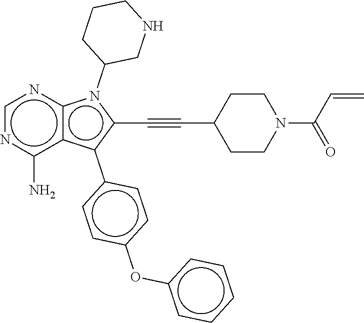 |
| I-61 | 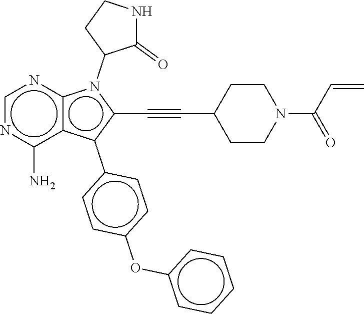 |
| I-62 | 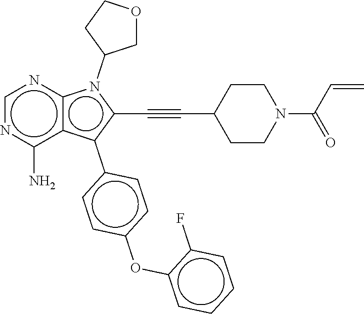 |
| I-63 | 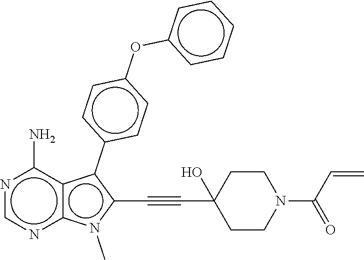 |
| I-64 | 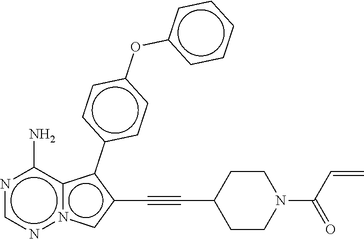 |
| I-65 | 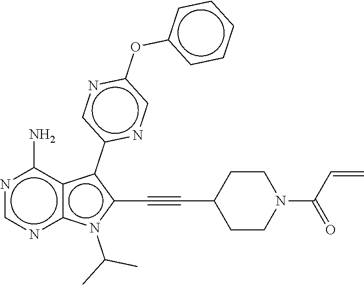 |
| I-66 | 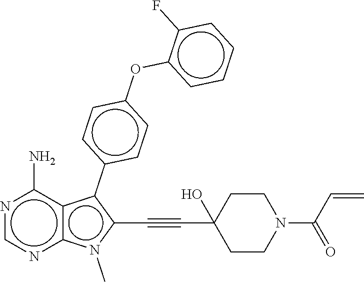 |
| I-67 | 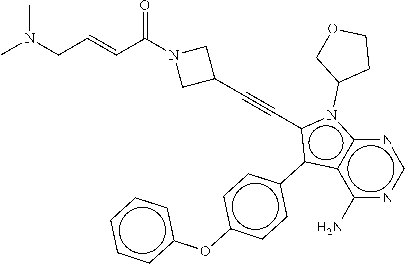 |
| I-68 | 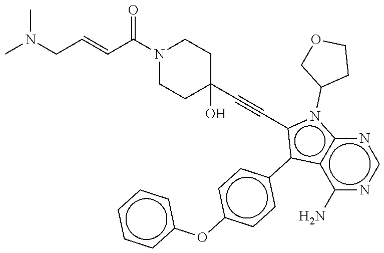 |
| I-69 | 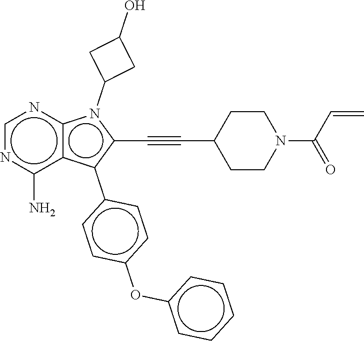 |
| I-70 | 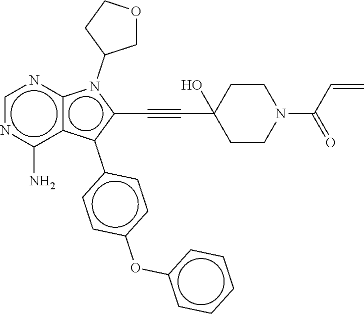 |
| I-71 | 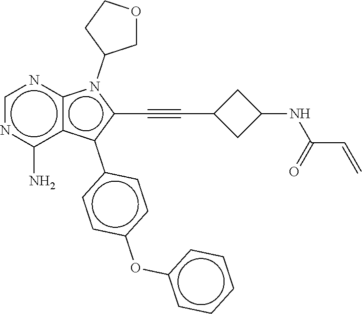 |

In some embodiments, the compound of the invention is selected from the group consisting of the compounds in Table 4, above, or a pharmaceutically acceptable salt thereof. In some embodiments, the compound of the invention is selected from the group consisting of a pharmaceutically acceptable salt of the compounds in Table 4, above.

In some embodiments, the compound of the invention is selected from the group consisting of the compounds in Tables 3-18, herein. In some embodiments, the compound of the invention is selected from the group consisting of the compounds in Tables 3-18, herein, or a pharmaceutically acceptable salt thereof.

In some embodiments, the compound of the invention is selected from the group consisting of the compounds in Table 19, below. In some embodiments, the compound of the invention is selected from the group consisting of the compounds in Table 19, below, or a pharmaceutically acceptable salt thereof.

In some embodiments, the present invention provides a compound in Table 19, wherein the compound is denoted as having a Src Biochemcial Assay Protocol IC₅₀ of “A”. In some embodiments, the present invention provides a compound in Table 19, wherein the compound is denoted as having a Src Biochemcial Assay Protocol IC₅₀ of “A” or “B”. In some embodiments, the present invention provides a compound in Table 19, wherein the compound is denoted as having a Src Biochemcial Assay Protocol IC₅₀ of “A” or “B” or “C”. In some embodiments, the present invention provides a compound in Table 19, wherein the compound is denoted as having a Src Biochemcial Assay Protocol IC₅₀ of “A” or “B” or “C” or “D”.

In some embodiments, the present invention provides a compound in Table 19, wherein the compound is denoted as having a Cell Viability Protocol IC₅₀ of “A”. In some embodiments, the present invention provides a compound in Table 19, wherein the compound is denoted as having a Cell Viability Protocol IC₅₀ of “A” or “B”. In some embodiments, the present invention provides a compound in Table 19, wherein the compound is denoted as having a Cell Viability Protocol IC₅₀ of “A” or “B” or “C”. In some embodiments, the present invention provides a compound in Table 19, wherein the compound is denoted as having a Cell Viability Protocol IC₅₀ of “A” or “B” or “C” or “D”.

In some embodiments, the present invention provides a compound in Table 19, wherein the compound is denoted as having a Cellular Proliferation Protocol IC₅₀ of “A”. In some embodiments, the present invention provides a compound in Table 19, wherein the compound is denoted as having a Cellular Proliferation Protocol IC₅₀ of “A” or “B”. In some embodiments, the present invention provides a compound in Table 19, wherein the compound is denoted as having a Cellular Proliferation Protocol IC₅₀ of “A” or “B” or “C”. In some embodiments, the present invention provides a compound in Table 19, wherein the compound is denoted as having a Cellular Proliferation Protocol IC₅₀ of “A” or “B” or “C” or “D”.

In some embodiments, the compound of the invention is selected from the group consisting of the compounds depicted in the Examples, below. In some embodiments, the compound of the invention is selected from the group consisting of the compounds in the Examples, below, or a pharmaceutically acceptable salt thereof. In some embodiments, the compound of the invention is selected from the group consisting of a pharmaceutically acceptable salt of the compounds in the Examples, below.

### EXEMPLARY PHARMACEUTICAL COMPOSITIONS

According to another embodiment, the invention provides a composition comprising a compound of this invention, or a pharmaceutically acceptable derivative thereof, and a pharmaceutically acceptable carrier, adjuvant, or vehicle. In some embodiments, the invention provides a pharmaceutical composition comprising a compound of this invention, and a pharmaceutically acceptable carrier. The amount of compound in compositions of this invention is such that is effective to measurably inhibit a Src kinase, or a mutant thereof, in a biological sample or in a patient. In certain embodiments, the amount of compound in compositions of this invention is such that it is effective to measurably inhibit a Src kinase, or a mutant thereof, in a biological sample or in a patient. In certain embodiments, a composition of this invention is formulated for administration to a patient in need of such composition. In some embodiments, a composition of this invention is formulated for oral administration to a patient.

The terms “subject” and “patient,” as used herein, means an animal (i.e., a member of the kingdom animal), preferably a mammal, and most preferably a human. In some embodiments, the subject is a human, mouse, rat, cat, monkey, dog, horse, or pig. In some embodiments, the subject is a human. In some embodiments, the subject is a mouse, rat, cat, monkey, dog, horse, or pig.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of a Src kinase, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal or vaginal administration. These can be prepared by mixing the agent with a suitable non-irritating excipient that is solid at room temperature but liquid at rectal or vaginal temperature and therefore will melt in the rectum or vagina to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the patient treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

The precise dose to be employed in the compositions will also depend on the route of administration, and should be decided according to the judgment of the practitioner and each subject's circumstances. In specific embodiments of the disclosure, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day. It will be recognized that any of the dosages listed herein may constitute an upper or lower dosage range, and may be combined with any other dosage to constitute a dosage range comprising an upper and lower limit.

In some embodiments, pharmaceutically acceptable compositions contain a provided compound and/or a pharmaceutically acceptable salt thereof at a concentration ranging from about 0.01 to about 90 wt %, about 0.01 to about 80 wt %, about 0.01 to about 70 wt %, about 0.01 to about 60 wt %, about 0.01 to about 50 wt %, about 0.01 to about 40 wt %, about 0.01 to about 30 wt %, about 0.01 to about 20 wt %, about 0.01 to about 2.0 wt %, about 0.01 to about 1 wt %, about 0.05 to about 0.5 wt %, about 1 to about 30 wt %, or about 1 to about 20 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient.

Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modern Pharmaceutics, 5^{th} Ed. (Alexander T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E. eds., Pharmaceutical Press (2012)) (each of which hereby incorporated by reference in its entirety).

The pharmaceutically acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain wetting or emulsifying agents, or pH buffering agents.

Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

### Exemplary Methods of the Invention

Compounds and compositions described herein are generally useful for the inhibition of kinase activity of one or more enzymes. In some embodiments the kinase inhibited by the compounds and methods of the invention is Src.

The activity of a compound utilized in this invention as an inhibitor of Src, or a mutant thereof, may be assayed in vitro, in vivo or in a cell line. In vitro assays include assays that determine inhibition of either the phosphorylation activity and/or the subsequent functional consequences, or ATPase activity of activated Src, or a mutant thereof. Alternate in vitro assays quantitate the ability of the inhibitor to bind to Src. Inhibitor binding may be measured by radiolabeling the inhibitor prior to binding, isolating the inhibitor/Src complex and determining the amount of radiolabel bound. Alternatively, inhibitor binding may be determined by running a competition experiment where new inhibitors are incubated with Src bound to known radioligands. Detailed conditions for assaying a compound utilized in this invention as an inhibitor of Src, or a mutant thereof, are set forth in the Examples below.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of Src and are therefore useful for treating one or more disorders associated with activity of Src or mutants thereof. Thus, in certain embodiments, the present invention provides a method for treating a Src-mediated disorder in a subject, comprising administering to the subject a compound of the present invention, or pharmaceutically acceptable composition thereof.

The terms “Src-mediated disease” or “Src-mediated condition”, as used herein mean any disease or other deleterious condition in which Src is known to play a role. The terms “Src-mediated disease” or “Src-mediated condition” also mean those diseases or conditions that are alleviated by treatment with a Src inhibitor. Such conditions include, without limitation, hypercalcemia, osteoporosis, osteoarthritis, cancer, symptomatic treatment of bone metastasis, and Paget's disease. Src protein kinase and its implication in various diseases has been described [Soriano, Cell, 1992, 69, 551; Soriano et al., Cell 1991, 64, 693; Takayanagi, J. Clin. Invest. 1999, 104, 137; Boschelli, Drugs of the Future 2000, 25 (7), 717; Talamonti, J. Clin. Invest. 1993, 91, 53; Lutz, Biochem. Biophys. Res. 1998, 243, 503; Rosen, J. Biol. Chem., 1986, 261, 13754; Bolen, Proc. Natl. Acad. Sci. USA 1987, 84, 2251; Masaki, Hepatology 1998, 27, 1257; Biscardi, Adv. Cancer Res. 1999, 76, 61; Lynch, Leukemia 1993, 7, 1416; Wiener, Clin. Cancer Res. 1999, 5, 2164; Staley, Cell Growth Diff, 1997, 8, 269]; the content of which are incorporated herein in their entireties by reference.

In some embodiments, the present invention provides a method for treating one or more disorders, diseases, and/or conditions wherein the disorder, disease, or condition includes, but is not limited to, a cellular proliferative disorder. In some embodiments, the present invention provides a method for treating one or more cellular proliferative disorders, said method comprising administering to a patient in need thereof, a compound of the present invention, or a pharmaceutically acceptable salt thereof. In some embodiments, the present invention provides a method for treating a proliferative disorder in a subject, said method comprising administering to the subject a therapeutically effective amount of a compound of the present invention, or a pharmaceutically acceptable salt thereof. In some embodiments, the present invention provides a method for treating one or more cellular proliferative disorders, said method comprising administering to a patient in need thereof, a pharmaceutical composition comprising an effective amount of a compound of the present invention, or a pharmaceutically acceptable salt thereof.

### Cellular Proliferative Disorders

The present invention features methods and compositions for the diagnosis and prognosis of cellular proliferative disorders (e.g., cancer) and the treatment of these disorders by targeting Src. Cellular proliferative disorders described herein include, e.g., cancer, obesity, and proliferation-dependent diseases. Such disorders may be diagnosed using methods known in the art.

### Cancer

Accordingly, in some embodiments, the present invention provides a method for treating cancer in a subject, said method comprising administering to the subject a therapeutically effective amount of a compound of the present invention, or a pharmaceutically acceptable salt thereof. In some embodiments, the cancer is biliary cancer or gall bladder cancer. In some embodiments, the biliary cancer is intrahepatic cholangiocarcinoma (ICC), or extrahepatic cholangiocarcinoma (ECC). In some embodiments, the ICC is IDH mutant ICC. In some embodiments, the cancer is liver cancer. In some embodiments, the liver cancer is hepatocellular carcinoma. In some embodiments, the cancer is colorectal cancer. In some embodiments, the colorectal cancer is metastatic colon cancer. In some embodiments, the cancer is a KRAS mutant cancer. In some embodiments, the KRAS mutant cancer is selected from lung cancer, pancreatic cancer, and ovarian cancer. In some embodiments, the KRAS mutant cancer is non-small cell lung adenocarcinoma (NSCLC). In some embodiments, the KRAS mutant cancer is pancreatic ductal adenocarcinoma (PDAC) or ovarian mucinous carcinoma (MCAS). In some embodiments, the cancer is a BRAF mutant cancer. In some embodiments, the BRAF mutant cancer is melanoma. In some embodiments, the cancer is an IDH mutant cancer. In some embodiments, the IDH mutant cancer is a solid tumor. In some embodiments, the IDH mutant cancer is a BRAF mutant melanoma that is resistant to treatment with a RAF inhibitor, an MEK inhibitor, or a RAF/MEK inhibitor.

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the present invention provides a method for treating a cancer that presents as a solid tumor, such as a sarcoma, carcinoma, or lymphoma, comprising the step of administering a disclosed compound, or a pharmaceutically acceptable salt thereof, to a patient in need thereof. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

Certain subtypes of hepatocellular carcinoma (HCC) having low alpha-fetoprotein (AFP) expression levels, such as the S1 subtype, are especially susceptible to Src inhibition. Hirschfield et al., Exp. & Mol. Med. (2018) 50, e419. In some embodiments, the present invention provides a method of treating hepatocellular carcinoma, comprising administering to a patient in need thereof a therapeutically effective amount of a Src inhibitor, wherein the hepatocellular carcinoma is comprised of cells of the S1 subtype. In some embodiments, the present invention provides a method of treating hepatocellular carcinoma, comprising administering to a patient in need thereof a therapeutically effective amount of a Src inhibitor, wherein the hepatocellular carcinoma is comprised of cells that express levels of alpha-fetoprotein lower than 400 ng/mL, 300 ng/mL, 200 ng/mL, or 100 ng/mL.

The present invention further features methods and compositions for the diagnosis, prognosis and treatment of viral-associated cancers, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892).

In some embodiments, the present invention provides a method for treating a tumor in a patient in need thereof, comprising administering to the patient any of the compounds, salts or pharmaceutical compositions described herein. In some embodiments, the tumor comprises any of the cancers described herein. In some embodiments, the tumor comprises melanoma cancer. In some embodiments, the tumor comprises breast cancer. In some embodiments, the tumor comprises lung cancer. In some embodiments the tumor comprises small cell lung cancer (SCLC). In some embodiments the tumor comprises non-small cell lung cancer (NSCLC).

In some embodiments, the tumor is treated by arresting further growth of the tumor. In some embodiments, the tumor is treated by reducing the size (e.g., volume or mass) of the tumor by at least 5%, 10%, 25%, 50%, 75%, 90% or 99% relative to the size of the tumor prior to treatment. In some embodiments, tumors are treated by reducing the quantity of the tumors in the patient by at least 5%, 10%, 25%, 50%, 75%, 90% or 99% relative to the quantity of tumors prior to treatment.

### Primary Immune Deficiencies

In some embodiments, the present invention provides a method for treating one or more disorders, diseases, and/or conditions wherein the disorder, disease, or condition includes, but is not limited to, a primary immunodeficiency disease or disorder, comprising administering to a patient in need thereof an effective amount of a disclosed compound. Primary immune deficiencies treatable by the methods of the present invention include: warts, hypogammaglobulinemia, infections, myelokathexis (WHIMs) syndrome; severe congenital neutropenia (SCN), especially those arising from G6PC3 deficiency (McDermott et al. (2010) Blood 116:2793-2802); GATA2 deficiency (Mono MAC syndrome) (Maciejweski-Duval et al. (2015) J. Leukoc. Biol. 5MA0815-288R (Epub. ahead of printing); idiopathic CD4+T lymphocytopenia (ICL); and Wiskott-Aldrich Syndrome.

### Neurodegenerative Diseases, Disorders or Conditions

Compounds as described herein are useful in the treatment of neurodegenerative diseases. In some embodiments, the neurodegenerative disease is selected from Alzheimer's Disease (AD), Parkinson's Disease (PD), Huntington's Disease (HD), Multiple Sclerosis (MS), amyotrophic lateral sclerosis (ALS), chronic traumatic encephalopathy (CTE), or a neurodegenerative condition caused by a virus, alcoholism, tumor, toxin, or repetitive brain injuries. In some embodiments, the neurodegenerative disease is selected from Alzheimer's Disease (AD), Parkinson's Disease (PD), Huntington's Disease (HD), Multiple Sclerosis (MS), amyotrophic lateral sclerosis (ALS), chronic traumatic encephalopathy (CTE). In some embodiments, the neurodegenerative disease is a neurodegenerative condition caused by a virus, alcoholism, tumor, toxin, or repetitive brain injuries.

In some embodiments, the neurodegenerative disease is Alzheimer's Disease (AD). In some embodiments, the neurodegenerative disease is Parkinson's Disease (PD). In some embodiments, the neurodegenerative disease is Huntington's Disease (HD). In some embodiments, the neurodegenerative disease is Multiple Sclerosis (MS). In some embodiments, the neurodegenerative disease is amyotrophic lateral sclerosis (ALS). In some embodiments, the neurodegenerative disease is chronic traumatic encephalopathy (CTE).

In some embodiments, the neurodegenerative disease is selected from Alzheimer's disease, amyotrophic lateral sclerosis (ALS), Parkinson's disease, Huntington's disease, brain aging, Friedreich's ataxia, multiple sclerosis, diabetic necrosis, ischaemia, and stroke.

In other embodiments, the neurodegenerative condition is due to toxic neuropathies, meningoencephalopathies, neurodegeneration caused by a genetic disorder, age-related neurodegeneration, or a vascular disease; or another disease disclosed in U.S. Pat. No. 8,691,775, which is hereby incorporated by reference.

### Inflammatory Diseases, Disorders or Conditions

Compounds as described herein are useful in the treatment of inflammatory or obstructive airways diseases, resulting, for example, in reduction of tissue damage, airways inflammation, bronchial hyperreactivity, remodeling or disease progression. In some embodiments, an inflammatory disease, disorder, or condition is inflammatory or obstructive airways diseases including, but not limited to, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Compounds as described herein are useful in the treatment of heteroimmune diseases. In some embodiments, an inflammatory disease, disorder, or condition is heteroimmune diseases including, but not limited to, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, such as therapy for or intended to restrict or abort symptomatic attack when it occurs, for example antiinflammatory or bronchodilatory. Prophylactic benefit in asthma may in particular be apparent in subjects prone to “morning dipping”. “Morning dipping” is a recognized asthmatic syndrome, common to a substantial percentage of asthmatics and characterised by asthma attack, e.g. between the hours of about 4 to 6 am, i.e. at a time normally substantially distant form any previously administered symptomatic asthma therapy.

In some embodiments, an inflammatory disease, disorder, or condition is selected from acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. In some embodiments, an inflammatory disease, disorder, or condition is bronchitis, wherein the bronchitis is of whatever type or genesis including, but not limited to, acute, arachidic, catarrhal, croupus, chronic or phthinoid bronchitis. In some embodiments, an inflammatory disease, disorder, or condition is pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

In some embodiments, an inflammatory disease, disorder, or condition is an eosinophil related disorder, e.g. eosinophilia. In some embodiments, an eosinophil related disorder is an eosinophil related disorder of the airways (e.g. involving morbid eosinophilic infiltration of pulmonary tissues) including hypereosinophilia as it effects the airways and/or lungs as well as, for example, eosinophil-related disorders of the airways consequential or concomitant to Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Compounds as described herein are also useful in the treatment of inflammatory or allergic conditions of the skin. In some embodiments, an inflammatory or allergic condition of the skin is selected from psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

In some embodiments, an inflammatory disease, disorder, or condition is a disease or condition having an inflammatory component, for example, diseases and conditions of the eye such as ocular allergy, conjunctivitis, keratoconjunctivitis sicca, and vernal conjunctivitis, diseases and conditions affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, Muckle-Wells syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments, an inflammatory disease, disorder, or condition is an inflammatory disease, disorder, or condition of the skin. In some embodiments, an inflammatory disease, disorder, or condition of the skin is selected from contact dermatitits, atompic dermatitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, and other inflammatory or allergic conditions of the skin.

In some embodiments, an inflammatory disease, disorder, or condition is selected from acute and chronic gout, chronic gouty arthritis, psoriasis, psoriatic arthritis, rheumatoid arthritis, Juvenile rheumatoid arthritis, Systemic jubenile idiopathic arthritis (SJIA), Cryopyrin Associated Periodic Syndrome (CAPS), Muckle-Wells syndrome, and osteoarthritis.

In some embodiments, an inflammatory disease, disorder, or condition is a TH17 mediated disease. In some embodiments, a TH17 mediated disease is selected from Systemic lupus erythematosus, Multiple sclerosis, and inflammatory bowel disease (including Crohn's disease or ulcerative colitis).

In some embodiments, an inflammatory disease, disorder, or condition is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

In some embodiments, an inflammatory disease, disorder, or condition is associated with transplantation. In some embodiments, an inflammatory disease, disorder, or condition is associated with organ transplantation, organ transplant rejection, and/or graft versus host disease.

In some embodiments, an inflammatory disease, disorder, or condition is an autoimmune disorder. In some embodiments an autoimmune disorder is type 1 diabetes, systemic lupus erythematosus, multiple sclerosis, psoriasis, Behçet's disease, POEMS syndrome, Crohn's disease, ulcerative colitis, ankylosing spondylitis, axial spondyloarthritis, primary biliary cirrhosis, autoimmune hepatitis, or inflammatory bowel disease.

In some embodiments, an inflammatory disease, disorder, or condition is an inflammatory disorder. In some embodiments, an inflammatory disorder is rheumatoid arthritis, asthma, chronic obstructive pulmonary disease, psoriasis, hepatomegaly, Crohn's disease, ulcerative colitis, ankylosing spondylitis, axial spondyloarthritis, primary biliary cirrhosis, polymyalgia rheumatica, giant cell arteritis, or inflammatory bowel disease.

### Routes of Administration

The compounds and compositions, according to the method of the present invention, may be administered using any amount and any route of administration effective for treating or lessening the severity of a cancer, an autoimmune disorder, a primary immune deficiency, a proliferative disorder, an inflammatory disorder, a neurodegenerative or neurological disorder, schizophrenia, a bone-related disorder, liver disease, or a cardiac disorder. The exact amount required will vary from subject to subject, depending on the species, age, and general condition of the subject, the severity of the disease or condition, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Inhibiting Src Activity

According to one embodiment, the invention relates to a method of inhibiting Src activity in a biological sample comprising the step of contacting said biological sample with a compound of this invention, or a composition comprising said compound.

According to another embodiment, the invention relates to a method of inhibiting Src, or a mutant thereof, activity in a biological sample comprising the step of contacting said biological sample with a compound of this invention, or a composition comprising said compound. In certain embodiments, the invention relates to a method of irreversibly inhibiting Src, or a mutant thereof, activity in a biological sample comprising the step of contacting said biological sample with a compound of this invention, or a composition comprising said compound. In some embodiments, the invention relates to a method of irreversibly inhibiting Src, or a mutant thereof, wherein a compound of this invention forms a covalent bond with Src, or a mutant thereof. In some embodiments, the invention relates to a method of irreversibly inhibiting Src, or a mutant thereof, wherein a compound of this invention forms a covalent bond between R⁶ of the compound and a cysteine of the Src, or a mutant thereof. In some embodiments, the invention relates to a method of irreversibly inhibiting Src, or a mutant thereof, wherein a compound of this invention forms a covalent bond between R⁶ of the compound and Cys277 of the Src, or a mutant thereof.

According to another embodiment, the invention relates to a Src, or a mutant thereof, irreversibly inhibited by a compound of this invention. In some embodiments, the invention relates to a Src, or a mutant thereof, covalently bonded to a compound of this invention. In some embodiments, the invention relates to a Src, or a mutant thereof, covalently bonded to a compound of this invention, wherein the covalent bond is between R⁶ of the compound and a cysteine of the Src, or a mutant thereof. In some embodiments, the invention relates to a Src, or a mutant thereof, covalently bonded to a compound of this invention, wherein the covalent bond is between R⁶ of the compound and Cys277 of the Src, or a mutant thereof.

The term “biological sample”, as used herein, includes, without limitation, cell cultures or extracts thereof; biopsied material obtained from a mammal or extracts thereof, and blood, saliva, urine, feces, semen, tears, or other body fluids or extracts thereof.

Another embodiment of the present invention relates to a method of inhibiting Src in a patient comprising the step of administering to said patient a compound of the present invention, or a composition comprising said compound.

According to another embodiment, the invention relates to a method of inhibiting Src, or a mutant thereof, activity in a patient comprising the step of administering to said patient a compound of the present invention, or a composition comprising said compound. According to certain embodiments, the invention relates to a method of irreversibly inhibiting Src, or a mutant thereof, activity in a patient comprising the step of administering to said patient a compound of the present invention, or a composition comprising said compound. In some embodiments, the invention relates to a method of irreversibly inhibiting Src, or a mutant thereof, activity in a patient comprising the step of administering to said patient a compound of the present invention, or a composition comprising said compound, wherein the compound forms a covalent bond with the Src, or a mutant thereof. In some embodiments, the invention relates to a method of irreversibly inhibiting Src, or a mutant thereof, activity in a patient comprising the step of administering to said patient a compound of the present invention, or a composition comprising said compound, wherein the compound forms a covalent bond between R⁶ of the compound and a cysteine of the Src, or a mutant thereof. In some embodiments, the invention relates to a method of irreversibly inhibiting Src, or a mutant thereof, activity in a patient comprising the step of administering to said patient a compound of the present invention, or a composition comprising said compound, wherein the compound forms a covalent bond between R⁶ of the compound and Cys277 of the Src, or a mutant thereof.

In other embodiments, the present invention provides a method for treating a disorder mediated by Src, or a mutant thereof, in a patient in need thereof, comprising the step of administering to said patient a compound according to the present invention or pharmaceutically acceptable composition thereof. Such disorders are described in detail herein. In some embodiments, the present invention provides a method for treating a disorder mediated by Src, or a mutant thereof, in a patient in need thereof, comprising the step of administering to said patient a compound according to the present invention or pharmaceutically acceptable composition thereof, wherein the compound irreversibly inhibits the Src, or a mutant thereof. In some embodiments, the present invention provides a method for treating a disorder mediated by Src, or a mutant thereof, in a patient in need thereof, comprising the step of administering to said patient a compound according to the present invention or pharmaceutically acceptable composition thereof, wherein the compound forms a covalent bond with the Src, or a mutant thereof. In some embodiments, the present invention provides a method for treating a disorder mediated by Src, or a mutant thereof, in a patient in need thereof, comprising the step of administering to said patient a compound according to the present invention or pharmaceutically acceptable composition thereof, wherein the compound forms a covalent bond between R⁶ of the compound and a cysteine of the Src, or a mutant thereof. In some embodiments, the present invention provides a method for treating a disorder mediated by Src, or a mutant thereof, in a patient in need thereof, comprising the step of administering to said patient a compound according to the present invention or pharmaceutically acceptable composition thereof, wherein the compound forms a covalent bond between R⁶ of the compound and Cys277 of the Src, or a mutant thereof.

Co-Administration with One or More Other Therapeutic Agent

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

As used herein, the term “combination,” “combined,” and related terms refers to the simultaneous or sequential administration of therapeutic agents in accordance with this invention. For example, a compound of the present invention may be administered with one or more other therapeutic agent simultaneously or sequentially in separate unit dosage forms or together in a single unit dosage form. Accordingly, the present invention provides a single unit dosage form comprising a compound of the current invention, one or more other therapeutic agent, and a pharmaceutically acceptable carrier, adjuvant, or vehicle.

The amount of a compound of the invention and one or more other therapeutic agent (in those compositions which comprise an additional therapeutic agent as described above) that may be combined with the carrier materials to produce a single dosage form will vary depending upon the host treated and the particular mode of administration. Preferably, a composition of the invention should be formulated so that a dosage of between 0.01-100 mg/kg body weight/day of a compound of the invention can be administered.

In those compositions which comprise one or more other therapeutic agent, the one or more other therapeutic agent and a compound of the invention may act synergistically. Therefore, the amount of the one or more other therapeutic agent in such compositions may be less than that required in a monotherapy utilizing only that therapeutic agent. In such compositions a dosage of between 0.01-1,000 μg/kg body weight/day of the one or more other therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approved for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Other Therapeutic Agents

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis); abemaciclib (Ly2835219, Eli Lilly); and trilaciclib (G1T28, G1 Therapeutics).

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, one or more other therapeutic agent is a platinum-based therapeutic, also referred to as platins. Platins cause cross-linking of DNA, such that they inhibit DNA repair and/or DNA synthesis, mostly in rapidly reproducing cells, such as cancer cells. In some embodiments, a platinum-based therapeutic is selected from cisplatin (Platinol®, Bristol-Myers Squibb); carboplatin (Paraplatin®, Bristol-Myers Squibb; also, Teva; Pfizer); oxaliplatin (Eloxitin® Sanofi-Aventis); nedaplatin (Aqupla®, Shionogi), picoplatin (Poniard Pharmaceuticals); and satraplatin (JM-216, Agennix).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is a proteasome inhibitor. Approved proteasome inhibitors useful in the present invention include bortezomib (Velcade®, Takeda); carfilzomib (Kyprolis®, Amgen); and ixazomib (Ninlaro®, Takeda).

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFβ trap compound (NCT02699515); and (NCT02517398). M7824 is comprised of a fully human IgG1 antibody against PD-L1 fused to the extracellular domain of human TGF-beta receptor II, which functions as a TGFβ “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™ Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™ Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cisplatin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MM1270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; Zd₆474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgG1 antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g. Patents International (e.g. IMS World Publications).

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1 h68/GLV-1 h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials. gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams ET. AL., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams ET. AL. In some embodiments, an immuno-oncology agent is a small molecule targeting an immuno-oncoloby target selected from those listed in Table 2 of Jerry L. Adams ET. AL. In some embodiments, an immuno-oncology agent is a small molecule agent selected from those listed in Table 2 of Jerry L. Adams ET. AL.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺(αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PDl antibody), CT-011 (anti-PDl antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMIP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

The invention now being generally described, it will be more readily understood by reference to the following examples, which are included merely for purposes of illustration of certain aspects and embodiments of the present invention, and are not intended to limit the invention.

### EXAMPLES

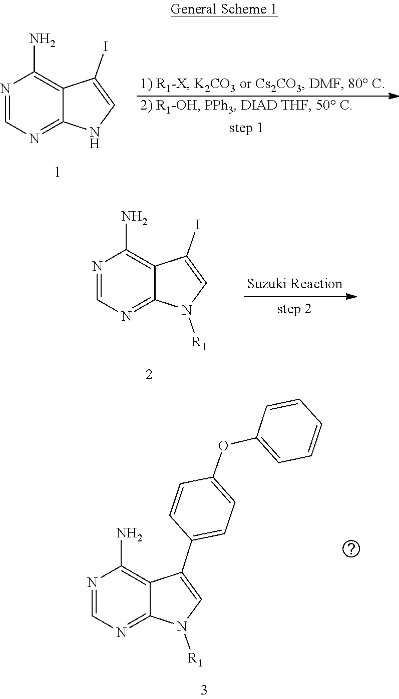

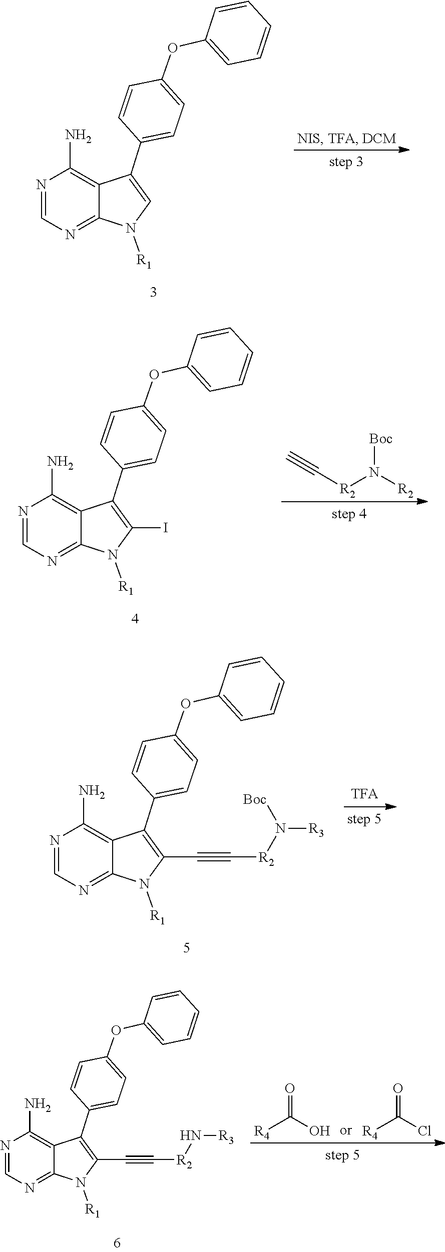

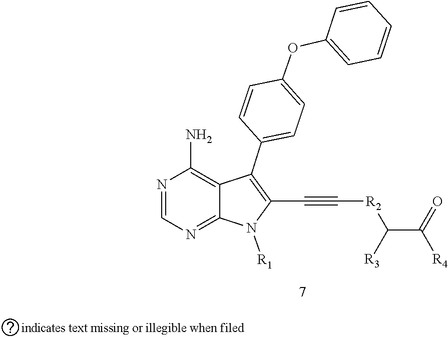

### Example 1

### 1-(4-((4-amino-7-isopropyl-5-(4-phenoxyphenyl)-7H-pyrrolo[2,3-d]pyrimidin-6-yl)ethynyl)piperidin-1-yl)prop-2-en-1-one (Compound I-8)

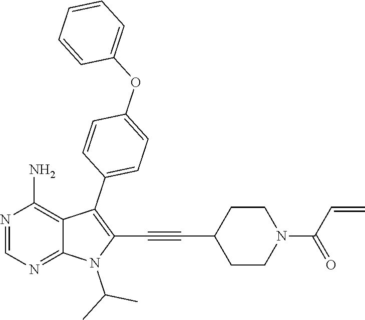 [SMILES: C=CC(=O)N1CCC(C#Cc2c(-c3ccc(Oc4ccccc4)cc3)c3c(N)ncnc3n2C(C)C)CC1]

### 5-iodo-7-isopropyl-7H-pyrrolo[2,3-d]pyrimidin-4-amine

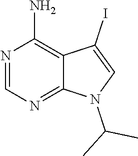

### Step 1

A round bottomed flask was charged with 5-iodo-7H-pyrrolo[2,3-d]pyrimidin-4-amine (12 g, 46.1 mmol), 2-iodopropane (11.7 g, 69.1 mmol), K₂CO₃ (19.0 g, 138 mmol) and a stirbar. DMF (100 mL) was added, and the solution was stirred for 1 h at 80° C. The reaction mixture was diluted with H₂O (200 mL), and the aqueous phase was extracted with ethyl acetate (300 mL) three times. The combined organic layers were washed with saturated brines, dried over sodium sulfate, filtered, and concentrated in vacuo. The resulting crude material was purified by silica gel chromatography (eluting with PE/EA=1/2). Concentration in vacuo resulted in 5-iodo-7-isopropyl-7H-pyrrolo [2,3-d]pyrimidin-4-amine (6.5 g, 46.6% yield) as a off-white amorphous solid.

### 7-isopropyl-5-(4-phenoxyphenyl)-7H-pyrrolo[2,3-d]pyrimidin-4-amine

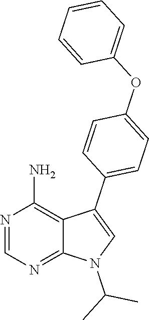 [SMILES: CC(C)n1cc(-c2ccc(Oc3ccccc3)cc2)c2c(N)ncnc21]

### Step 2

A resealable reaction vial was charged with 5-iodo-7-isopropyl-7H-pyrrolo[2,3-d]pyrimidin-4-amine (4.5 g, 14.8 mmol), (4-phenoxyphenyl)boronic acid (3.46 g, 16.2 mmol), Pd(DtBPF)Cl₂ (1 g, 1.48 mmol), K₂CO₃ (6.12 g, 44.4 mmol), and a stirbar before being evacuated and purged with nitrogen three times. Dioxane (80 mL) and H₂O (20 mL) was added, and the mixture was stirred for 1 h at 90° C. The reaction mixture was diluted with H₂O (200 mL), and the aqueous phase was extracted with ethyl acetate (300 mL) three times. The combined organic layers were washed with brine, dried over sodium sulfate, filtered, and concentrated in vacuo. The resulting crude material was purified by silica gel chromatography (eluting with PE/EA=1/1). Concentration in vacuo resulted in 7-isopropyl-5-(4-phenoxyphenyl)-7H-pyrrolo[2,3-d]pyrimidin-4-amine (4.20 g, 82% yield) as a off-white amorphous solid.

### 6-iodo-7-isopropyl-5-(4-phenoxyphenyl)-7H-pyrrolo[2,3-d]pyrimidin-4-amine

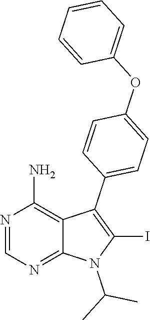 [SMILES: CC(C)n1c(I)c(-c2ccc(Oc3ccccc3)cc2)c2c(N)ncnc21]

### Step 3

A resealable reaction vial was charged with 7-isopropyl-5-(4-phenoxyphenyl)-7H-pyrrolo[2,3-d]pyrimidin-4-amine (4.2 g, 12.1 mmol), 1-iodopyrrolidine-2,5-dione (4.07 g, 18.1 mmol), DCM (100 mL), TFA (2.75 g, 24.2 mmol) and a stirbar before being evacuated and purged with nitrogen three times. The mixture was stirred for 2 h at room temperature. The reaction mixture was diluted with aqueous Na₂SO₃ (150 mL), and the aqueous phase was extracted with ethyl acetate (150 mL) three times. The combined organic layers were washed with saturated brines, dried over sodium sulfate, filtered, and concentrated in vacuo. The resulting crude material was purified by silica gel chromatography (eluting with PE/EA=3/1). Concentration in vacuo resulted in 6-iodo-7-isopropyl-5-(4-phenoxyphenyl)-7H-pyrrolo[2,3-d]pyrimidin-4-amine (2.75 g, 48% yield) as a yellow amorphous solid.

### tert-butyl 4-((4-amino-7-isopropyl-5-(4-phenoxyphenyl)-7H-pyrrolo[2,3-d]pyrimidin-6-yl)ethynyl)piperidine-1-carboxylate

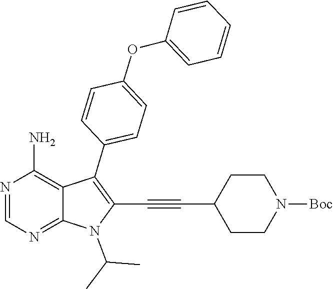 [SMILES: CC(C)n1c(C#CC2CCN(C(=O)OC(C)(C)C)CC2)c(-c2ccc(Oc3ccccc3)cc2)c2c(N)ncnc21]

### Step 4

A resealable reaction vial was charged with 6-iodo-7-isopropyl-5-(4-phenoxyphenyl)-7H-pyrrolo[2,3-d]pyrimidin-4-amine (1 g, 2.12 mmol), tert-butyl-4-ethynylpiperidine-1-carboxylate (887 mg, 4.24 mmol), Pd(PPh₃)₂Cl₂ (296 mg, 424 μmol), CuI (161 mg, 848 μmol), TEA (1.07 g, 10.6 mmol), THF (20 mL) was added and a stirbar before being evacuated and purged with nitrogen three times. The mixture was stirred for 4 h at room temperature. The reaction mixture was diluted with H₂O (100 mL), and the aqueous phase was extracted with ethyl acetate (150 mL) three times. The combined organic layers were washed with saturated brines, dried over sodium sulfate, filtered, and concentrated in vacuo. The resulting crude material was purified by silica gel chromatography (PE/EA=2/1). Concentration in vacuo resulted in tert-butyl 4-((4-amino-7-isopropyl-5-(4-phenoxyphenyl)-7H-pyrrolo[2,3-d]pyrimidin-6-yl)ethynyl)piperidine-1-carboxylate (0.9 g, 77% yield) as a yellow amorphous solid.

### 7-isopropyl-5-(4-phenoxyphenyl)-6-(piperidin-4-ylethynyl)-7H-pyrrolo[2,3-d]pyrimidin-4-amine

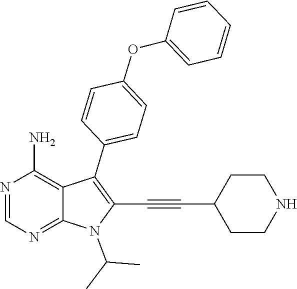 [SMILES: CC(C)n1c(C#CC2CCNCC2)c(-c2ccc(Oc3ccccc3)cc2)c2c(N)ncnc21]

### Step 5

A round bottomed flask was charged with tert-butyl 4-((4-amino-7-isopropyl-5-(4-phenoxyphenyl)-7H-pyrrolo[2,3-d]pyrimidin-6-yl)ethynyl)piperidine-1-carboxylate (1 g, 1.81 mmol) and a stirbar. DCM (10 mL) and TFA (2 mL) was added, and the solution was stirred for 1 h at room temperature. The PH of the mixture was adjusted to 9-10 with Na₂CO₃ and the aqueous phase was extracted with EA (100 mL) three times. The combined organic layers were washed with brine, dried over sodium sulfate, filtered, and concentrated in vacuo. 7-isopropyl-5-(4-phenoxyphenyl)-6-(piperidin-4-ylethynyl)-7H-pyrrolo[2,3-d]pyrimidin-4-amine (750 mg, 91% yield) as a yellow amorphous oil.

### 1-(4-((4-amino-7-isopropyl-5-(4-phenoxyphenyl)-7H-pyrrolo[2,3-d]pyrimidin-6-yl)ethynyl)piperidin-1-yl)prop-2-en-1-one

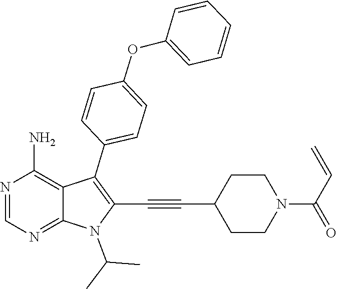 [SMILES: C=CC(=O)N1CCC(C#Cc2c(-c3ccc(Oc4ccccc4)cc3)c3c(N)ncnc3n2C(C)C)CC1]

### Step 6

A round bottomed flask was charged with 7-isopropyl-5-(4-phenoxyphenyl)-6-(piperidin-4-ylethynyl)-7H-pyrrolo[2,3-d]pyrimidin-4-amine (160 mg, 354 μmol), DCM (5 mL), TEA (107 mg, 1.06 mmol) and a stirbar. The solution was cooled to 0° C., then acryloyl chloride (32.0 mg, 354 μmol) was added, and the mixture was stirred for 1 h at 0° C. The reaction mixture was quenched with MeOH (1 mL), and the solution was concentrated in vacuo. The resulting crude material was purified by HPLC. Column: XBridge Prep C18 OBD Column, 5 um, 19*150 mm; Mobile Phase A:Water (10 MMOL/L NH₄HCO₃), Mobile Phase B: ACN; Flow rate: 20 mL/min; Gradient: 45% B to 80% B in 7 min; 254&220 nm; Rt: 6.5 min. Lyophilization yielded 1-(4-((4-amino-7-isopropyl-5-(4-phenoxyphenyl)-7H-pyrrolo[2,3-d]pyrimidin-6-yl)ethynyl)piperidin-1-yl)prop-2-en-1-one (54 mg, 30.3%) as a off-white amorphous solid.

### Example 2

### (E)-1-(4-((4-amino-7-isopropyl-5-(4-phenoxyphenyl)-7H-pyrrolo[2,3-d]pyrimidin-6-yl)ethynyl)piperidin-1-yl)-4-(dimethylamino)but-2-en-1-one (Compound I-48)

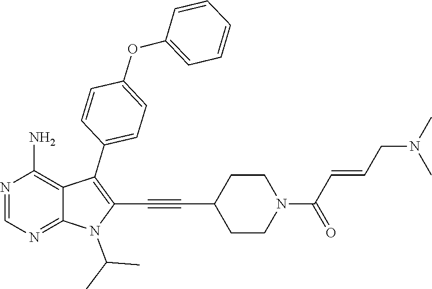 [SMILES: CC(C)n1c(C#CC2CCN(C(=O)/C=C/CN(C)C)CC2)c(-c2ccc(Oc3ccccc3)cc2)c2c(N)ncnc21]

### Step 1

A round bottomed flask was charged with 7-isopropyl-5-(4-phenoxyphenyl)-6-(piperidin-4-ylethynyl)-7H-pyrrolo[2,3-d]pyrimidin-4-amine (100 mg, 221 μmol), (E)-4-(dimethylamino)but-2-enoic acid hydrochloride (55 mg, 331 μmol), DIEA (141 mg, 1.10 mmol), HATU (100 mg, 265 μmol) and a stirbar. DMF (3 mL) was added, and the mixture was stirred for 1 h at room temperature. The reaction mixture was diluted with water (3 mL), and the aqueous phase was extracted with EA (15 mL) three times. The combined organic layers were washed with brine, dried over sodium sulfate, filtered, and concentrated in vacuo. The resulting crude material was purified by HPLC. Column: XBridge Prep OBD C18 Column 30×150 mm 5 um; Mobile Phase A:Water (10 MMOL/L NH₄HCO₃), Mobile Phase B: ACN; Flow rate: 60 mL/min; Gradient: 30% B to 65% B in 7 min; 220 nm; Rt: 6.52 min. Lyophilization yielded (E)-1-(4-((4-amino-7-isopropyl-5-(4-phenoxyphenyl)-7H-pyrrolo[2,3-d]pyrimidin-6-yl)ethynyl)piperidin-1-yl)-4-(dimethylamino)but-2-en-1-one (44.1 mg, 35.5%) as a off-white amorphous solid.

### Example 3

### 1-(4-((4-amino-5-(4-phenoxyphenyl)-7-(tetrahydro-2H-pyran-4-yl)-7H-pyrrolo[2,3-d]pyrimidin-6-yl)ethynyl)piperidin-1-yl)prop-2-en-1-one (Compound I-41)

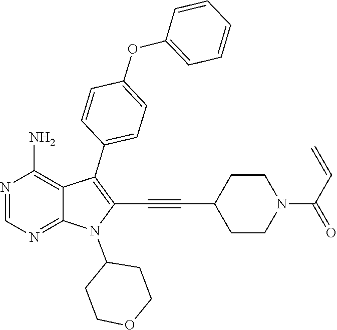 [SMILES: C=CC(=O)N1CCC(C#Cc2c(-c3ccc(Oc4ccccc4)cc3)c3c(N)ncnc3n2C2CCOCC2)CC1]

### 5-iodo-7-(tetrahydro-2H-pyran-4-yl)-7H-pyrrolo[2,3-d]pyrimidin-4-amine

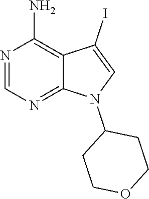 [SMILES: Nc1ncnc2c1c(I)cn2C1CCOCC1]

### Step 1

A round bottomed flask was charged with 5-iodo-7H-pyrrolo[2,3-d]pyrimidin-4-amine (800 mg, 3.07 mmol), oxan-4-ol (1.56 g, 15.3 mmol), PPh₃ (2.41 g, 9.20 mmol) and a stirbar, THF (12 mL) was added, DIAD (1.85 g, 9.20 mmol) was dropped at 0° C. The solution was stirred at 50° C. for 2.5 hours under N₂ atmosphere. The resulting crude material was purified by HPLC (acetonitrile/water). yielded 5-iodo-7-(oxan-4-yl)-7H-pyrrolo[2,3-d]pyrimidin-4-amine (310 mg, 900 μmol) as a off-white amorphous solid.

### Example 4

### N-(3-(4-amino-7-methyl-5-(4-phenoxyphenyl)-7H-pyrrolo[2,3-d]pyrimidin-6-yl)prop-2-ynyl)-N-methylacrylamide (Compound I-72)

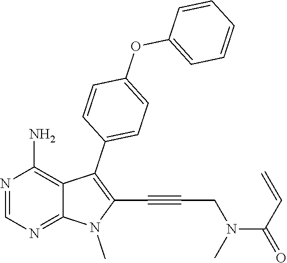 [SMILES: C=CC(=O)N(C)CC#Cc1c(-c2ccc(Oc3ccccc3)cc2)c2c(N)ncnc2n1C]

### tert-butyl methyl(prop-2-ynyl)carbamate

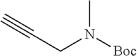

### Step 1

A round bottomed flask was charged with tert-butyl N-(prop-2-yn-1-yl)carbamate (2 g, 12.8 mmol), dimethylformamide (20 mL) was added, and the solution was stirred at 0° C. under a N₂ atmosphere. NaH (460 mg, 19.2 mmol) was added to the reaction mixture, and the solution was stirred at 0° C. for 20 min. CH₃I (2.72 g, 19.2 mmol) was added to the reaction mixture, and the solution was stirred at 0° C. for 3 hours. The reaction mixture was dropped into 100 mL of water, then extracted with EtOAc (3×100 mL). The organic layers were combined, dried and concentrated under vacuum. The resulting crude material was purified by silica gel chromatography. Concentration in vacuo resulted in tert-butyl N-methyl-N-(prop-2-yn-1-yl)carbamate (2.00 g, 11.8 mmol) as a off-white oil.

### Example 5 (Chiral Separation)

### (S,E)-1-(4-((4-amino-5-(4-phenoxyphenyl)-7-(tetrahydrofuran-3-yl)-7H-pyrrolo[2,3-d]pyrimidin-6-yl)ethynyl)piperidin-1-yl)-4-morpholinobut-2-en-1-one (Compound I-73)

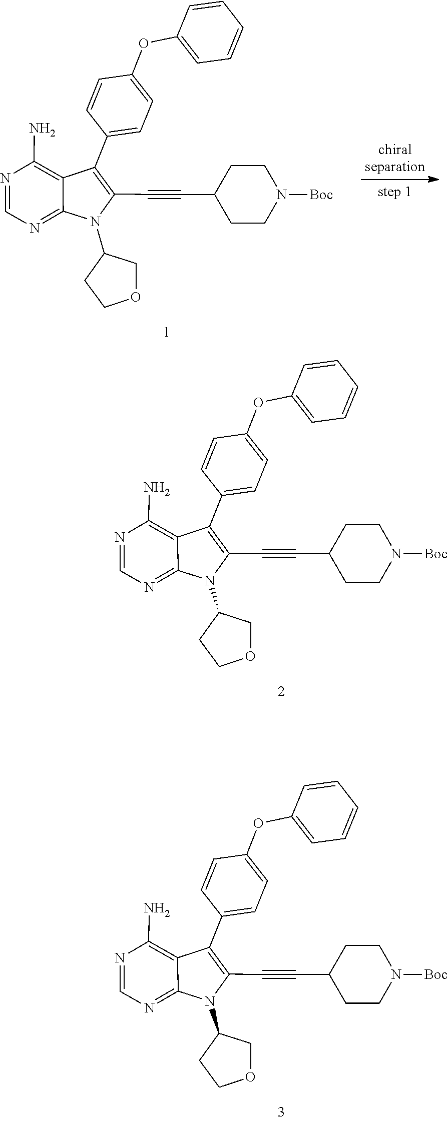 [SMILES: CC(C)(C)OC(=O)N1CCC(C#Cc2c(-c3ccc(Oc4ccccc4)cc3)c3c(N)ncnc3n2C2CCOC2)CC1 ; CC(C)(C)OC(=O)N1CCC(C#Cc2c(-c3ccc(Oc4ccccc4)cc3)c3c(N)ncnc3n2[C@H]2CCOC2)CC1 ; CC(C)(C)OC(=O)N1CCC(C#Cc2c(-c3ccc(Oc4ccccc4)cc3)c3c(N)ncnc3n2[C@@H]2CCOC2)CC1]

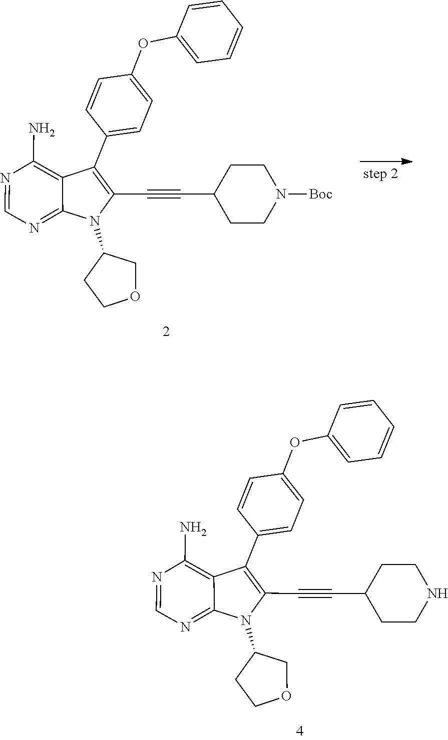 [SMILES: CC(C)(C)OC(=O)N1CCC(C#Cc2c(-c3ccc(Oc4ccccc4)cc3)c3c(N)ncnc3n2[C@H]2CCOC2)CC1 ; Nc1ncnc2c1c(-c1ccc(Oc3ccccc3)cc1)c(C#CC1CCNCC1)n2[C@H]1CCOC1]

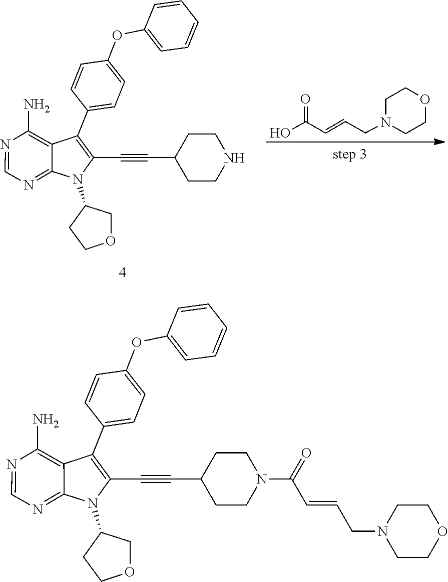 [SMILES: Nc1ncnc2c1c(-c1ccc(Oc3ccccc3)cc1)c(C#CC1CCNCC1)n2[C@H]1CCOC1 ; O=C(O)/C=C/CN1CCOCC1 ; Nc1ncnc2c1c(-c1ccc(Oc3ccccc3)cc1)c(C#CC1CCN(C(=O)/C=C/CN3CCOCC3)CC1)n2[C@H]1CCOC1]

### (S)-tert-butyl 4-((4-amino-5-(4-phenoxyphenyl)-7-(tetrahydrofuran-3-yl)-7H-pyrrolo[2,3-d]pyrimidin-6-yl)ethynyl)piperidine-1-carboxylate

### (R)-tert-butyl 4-((4-amino-5-(4-phenoxyphenyl)-7-(tetrahydrofuran-3-yl)-7H-pyrrolo[2,3-d]pyrimidin-6-yl)ethynyl)piperidine-1-carboxylate

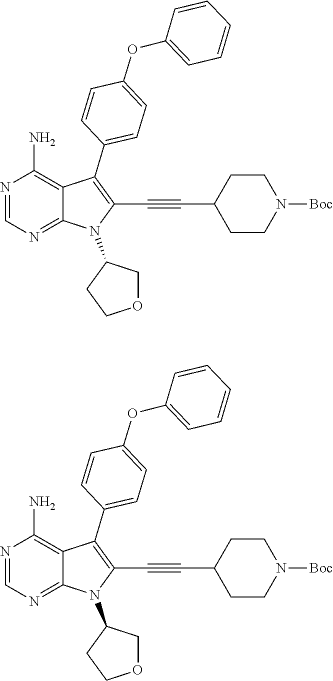 [SMILES: CC(C)(C)OC(=O)N1CCC(C#Cc2c(-c3ccc(Oc4ccccc4)cc3)c3c(N)ncnc3n2[C@H]2CCOC2)CC1 ; CC(C)(C)OC(=O)N1CCC(C#Cc2c(-c3ccc(Oc4ccccc4)cc3)c3c(N)ncnc3n2[C@@H]2CCOC2)CC1]

### Step 1

The resulting crude material was chiral separation by HPLC Enantiocel-C1, 5*25 cm, 5 um; Mobile Phase A:CO₂:50, Mobile Phase B: MeOH-Preparative:50; Flow rate: 180 mL/min; 220 nm; RT1:9.97; RT2:11.91. Lyophilization yielded tert-butyl-4-(2-{4-amino-7-[(3S)-oxolan-3-yl]-5-(4-phenoxyphenyl)-7H-pyrrolo[2,3-d]pyrimidin-6-yl}ethynyl)piperidine-1-carboxylate (230 mg, 46.0%) as a white amorphous solid and tert-butyl 4-(2-{4-amino-7-[(3R)-oxolan-3-yl]-5-(4-phenoxyphenyl)-7H-pyrrolo[2,3-d]pyrimidin-6-yl}ethynyl)piperidine-1-carboxylate (230 mg, 46.0%) as a white amorphous solid.

### (S)-5-(4-phenoxyphenyl)-6-(piperidin-4-ylethynyl)-7-(tetrahydrofuran-3-yl)-7H-pyrrolo[2,3-d]pyrimidin-4-amine

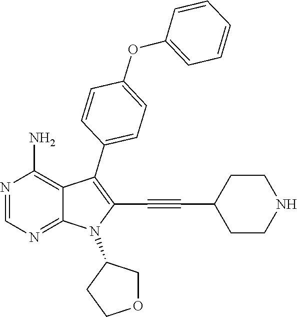 [SMILES: Nc1ncnc2c1c(-c1ccc(Oc3ccccc3)cc1)c(C#CC1CCNCC1)n2[C@H]1CCOC1]

### Step 2

A round bottomed flask was charged with tert-butyl 4-{2-[4-amino-7-(oxolan-3-yl)-5-(4-phenoxyphenyl)-7H-pyrrolo[2,3-d]pyrimidin-6-yl]ethynyl}piperidine-1-carboxylate (230 mg, 465 μmol), DCM (4 mL), TFA (1 mL) and a stirbar. The solution was stirred for 1 h at room temperature. The reaction mixture was diluted with H₂O (10 mL), and the pH of the aqueous phase was adjusted to 9-10 with Na₂CO₃ solution. The aqueous phase was extracted with EA (50 mL) three times. The combined organic layers were washed with brines, dried over sodium sulfate, filtered, and concentrated in vacuo resulted in (S)-5-(4-phenoxyphenyl)-6-(piperidin-4-ylethynyl)-7-(tetrahydrofuran-3-yl)-7H-pyrrolo[2,3-d]pyrimidin-4-amine (160 mg, 85.0%) as a yellow amorphous solid.

### (S,E)-1-(4-((4-amino-5-(4-phenoxyphenyl)-7-(tetrahydrofuran-3-yl)-7H-pyrrolo[2,3-d]pyrimidin-6-yl)ethynyl)piperidin-1-yl)-4-morpholinobut-2-en-1-one

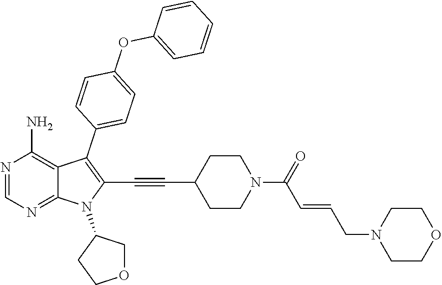 [SMILES: Nc1ncnc2c1c(-c1ccc(Oc3ccccc3)cc1)c(C#CC1CCN(C(=O)/C=C/CN3CCOCC3)CC1)n2[C@H]1CCOC1]

### Step 3

A round bottomed flask was charged with 7-(oxolan-3-yl)-5-(4-phenoxyphenyl)-6-[2-(piperidin-4-yl)ethynyl]-7H-pyrrolo[2,3-d]pyrimidin-4-amine (50 mg, 104 μmol), (2E)-4-(morpholin-4-yl)but-2-enoic acid (17.8 mg, 104 μmol), HATU (47.1 mg, 124 μmol), DMF (3 mL), DIEA (40.2 mg, 312 μmol) and a stirbar. The solution was stirred for 1 h at room temperature. The resulting crude material was purified by HPLC Column: XBridge Prep C18 OBD Column 19×150 mm 5 um; Mobile Phase A:Water (10 MMOL/L NH₄HCO₃), Mobile Phase B: ACN; Flow rate: 20 mL/min; Gradient: 48% B to 67% B in 7 min; 254/220 nm; Rt: 6.7 min. Lyophilization yielded (S,E)-1-(4-((4-amino-5-(4-phenoxyphenyl)-7-(tetrahydrofuran-3-yl)-7H-pyrrolo[2,3-d]pyrimidin-6-yl)ethynyl)piperidin-1-yl)-4-morpholinobut-2-en-1-one (23.0 mg, 34.9%) as a white amorphous solid.

Compounds prepared by substantially the same methods, and their characterization data, are listed in Table 5, below.

TABLE 5
Selected Compounds

| | | Proton NMR & |
|---|---|---|
| Compound | Structure | Mass Spec. (obs.) |
| 1-(4-((4-amino-7- isopropyl-5-(4- phenoxyphenyl)-7H- pyrrolo[2,3- d]pyrimidin-6- yl)ethynyl)piperidin- 1-yl)prop-2-en-1-one | 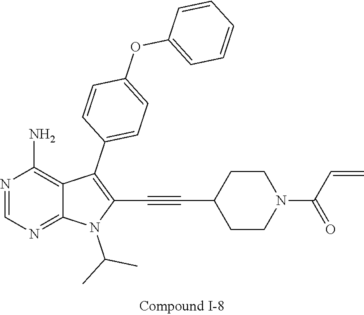 [SMILES: C=CC(=O)N1CCC(C#Cc2c(-c3ccc(Oc4ccccc4)cc3)c3c(N)ncnc3n2C(C)C)CC1] | ¹H NMR (300 MHz, DMSO-d₆) δ 8.16 (s, 1H), 7.51-7.40 (m, 4H), 7.20-7.07 (m, 5H), 6.79 (dd, J = 16.7, 10.5 Hz, 1H), 6.10 (dd, J = 16.7, 2.5 Hz, 1H), 5.67 (dd, J = 10.4, 2.5 Hz, 1H), 5.12 (p, J = 7.0 Hz, 1H), 3.65 (s, 2H), 3.43-3.39 (m, 2H), 3.03 (m, 1H), 1.80 (s, 2H), 1.62 (d, J = 6.9 Hz, 6H), 1.53 (s, 2H). MS: 506.3 |
| 1-(4-((4-amino-7- isopropyl-5-(4- phenoxyphenyl)-7H- pyrrolo[2,3- d]pyrimidin-6- yl)ethynyl)piperidin- 1-yl)-2- chloroethanone | 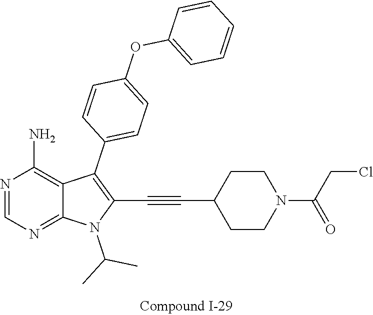 [SMILES: CC(C)n1c(C#CC2CCN(C(=O)CCl)CC2)c(-c2ccc(Oc3ccccc3)cc2)c2c(N)ncnc21] | ¹H NMR (300 MHz, Chloroform-d) δ 8.33 (s, 1H), 7.50-7.47 (m, 2H), 7.42-7.37 (m, 2H), 7.20-7.17 (m, 1H), 7.15-7.06 (m, 4H), 5.22 (q, J = 6.9 Hz, 1H), 5.18 (s, 2H), 4.06 (s, 2H), 3.58 (ddd, J = 11.7, 7.7, 3.7 Hz, 1H), 3.56 (td, J = 7.1, 3.7 Hz, 2H), 3.44-3.35 (m, 1H), 2.98 (tt, J = 7.4, 4.0 Hz, 1H), 1.92-1.84 (m, 2H), 1.78-1.70 (m, 2H), 1.69 (d, J = 6.9 Hz, 6H). MS: 528.3 |
| N-(4-((4-amino-7- isopropyl-5-(4- phenoxyphenyl)-7H- pyrrolo[2,3- d]pyrimidin-6- yl)ethynyl)cyclo- hexyl)acrylamide | 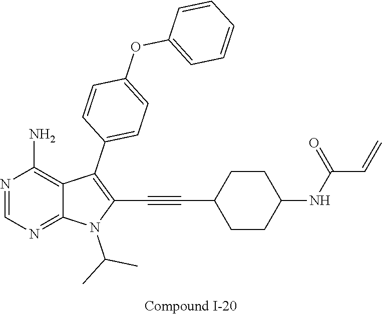 [SMILES: C=CC(=O)NC1CCC(C#Cc2c(-c3ccc(Oc4ccccc4)cc3)c3c(N)ncnc3n2C(C)C)CC1] | ¹H NMR (300 MHz, DMSO) δ 8.17 (s, 1H), 7.99 (d, J = 7.7 Hz, 1H), 7.48 (dt, J = 24.0, 8.2 Hz, 4H), 7.16 (dd, J = 21.0, 7.8 Hz, 5H), 6.22 (dd, J = 17.1, 10.0 Hz, 1H), 6.08 (dd, J = 17.1, 2.7 Hz, 1H), 5.57 (dd, J = 9.9, 2.6 Hz, 1H), 5.14 (p, J = 6.7 Hz, 1H), 3.64 (m, 1H), 2.62 (d, J = 11.1 Hz, 1H), 1.97 (d, J = 12.6 Hz, 2H), 1.82 (d, J = 12.0 Hz, 2H), 1.64 (d, J = 6.9 Hz, 6H), 1.46 (q, J = 12.1 Hz, 2H), 1.35-1.17 (m, 2H). MS: 520.3 |
| 7-isopropyl-5-(4- phenoxyphenyl)-6- ((1- (vinylsulfonyl) azetidin-3-yl)ethynyl)- 7H-pyrrolo[2,3- d]pyrimidin-4-amine | 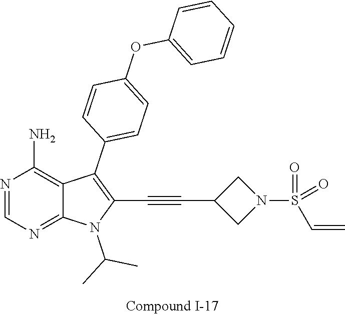 [SMILES: C=CS(=O)(=O)N1CC(C#Cc2c(-c3ccc(Oc4ccccc4)cc3)c3c(N)ncnc3n2C(C)C)C1] | ¹H NMR (400 MHz, DMSO-d₆) δ 8.18 (s, 1H), 7.52-7.50 (m, 2H), 7.45 (ddd, J = 10.3, 6.0, 2.3 Hz, 2H), 7.21-7.12 (m, 5H), 7.02 (dd, J = 16.6, 10.1 Hz, 1H), 6.26 (d, J = 10.0 Hz, 1H), 6.19 (d, J = 16.6 Hz, 1H), 5.09 (p, J = 6.9 Hz, 1H), 4.09 (d, J = 5.9 Hz, 2H), 3.77 (t, J = 3.7 Hz, 3H), 1.62 (d, J = 6.9 Hz, 6H). MS: 514.2 |
| N-(4-((4-amino-7- isopropyl-5-(4- phenoxyphenyl)-7H- pyrrolo[2,3- d]pyrimidin-6- yl)ethynyl)cyclohexyl) ethenesulfonamide | 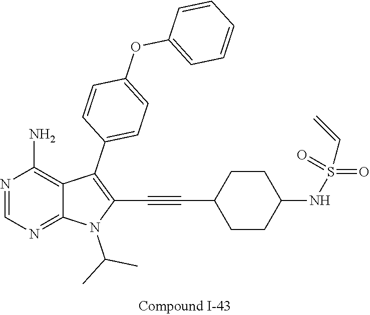 [SMILES: C=CS(=O)(=O)NC1CCC(C#Cc2c(-c3ccc(Oc4ccccc4)cc3)c3c(N)ncnc3n2C(C)C)CC1] | ¹H NMR (400 MHz, Methanol) δ 8.14 (s, 1H), 7.55-7.47 (m, 2H), 7.46-7.37 (m, 2H), 7.22-7.13 (m, 1H), 7.18-7.05 (m, 4H), 6.67 (dd, J = 16.5, 9.9 Hz, 1H), 6.15 (d, J = 16.6 Hz, 1H), 5.92 (d, J = 9.9 Hz, 1H), 5.19 (p, J = 6.9 Hz, 1H), 3.10 (t, J = 10.1 Hz, 1H), 2.54 (d, J = 10.5 Hz, 1H), 2.07-1.93 (m, 4H), 1.68 (d, J = 7.0 Hz, 6H), 1.55-1.28 (m, 4H). MS: 556.2 |
| 1-(3-((4-amino-7- isopropyl-5-(4- phenoxyphenyl)-7H- pyrrolo[2,3- d]pyrimidin-6- yl)ethynyl)azetidin- 1-yl)prop-2-en-1-one | 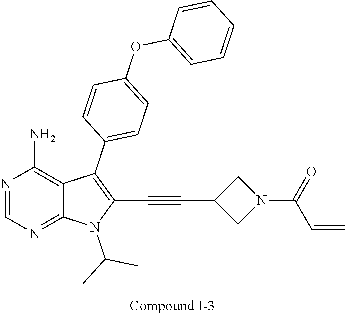 [SMILES: C=CC(=O)N1CC(C#Cc2c(-c3ccc(Oc4ccccc4)cc3)c3c(N)ncnc3n2C(C)C)C1] | ¹H NMR (400 MHz, DMSO-d₆) δ 8.17 (s, 1H), 7.52-7.50 (m, 2H), 7.45-7.41 (m, 2H), 7.19 (dtt, J = 8.5, 7.1, 1.1 Hz, 1H), 7.13-7.09 (m, 4H), 6.29 (dd, J = 17.0, 10.2 Hz, 1H), 6.11 (dd, J = 17.0, 2.2 Hz, 1H), 5.67 (dd, J = 10.3, 2.3 Hz, 1H), 5.10 (p, J = 6.8 Hz, 1H), 4.53 (t, J = 8.4 Hz, 1H), 4.25 (t, J = 8.9 Hz, 1H), 4.21-4.13 (m, 1H), 3.82 (ddt, J = 23.1, 8.7, 6.2 Hz, 2H), 1.62 (d, J = 6.9 Hz, 6H). MS: 478.2 |
| N-(4-((4-amino-7- isopropyl-5-(4- phenoxyphenyl)-7H- pyrrolo[2,3- d]pyrimidin-6- yl)ethynyl)cyclohexyl)- 2-chloroacetamide | 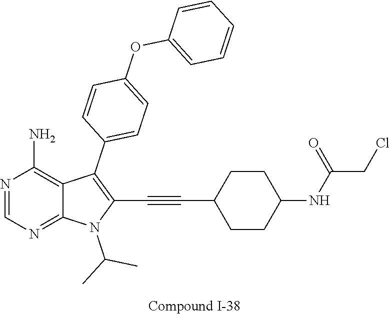 [SMILES: CC(C)n1c(C#CC2CCC(NC(=O)CCl)CC2)c(-c2ccc(Oc3ccccc3)cc2)c2c(N)ncnc21] | ¹H NMR (400 MHz, DMSO) δ 8.16 (s, 1H), 8.11 (d, J = 8.0, 1H), 7.56- 7.45 (m, 2H), 7.48-7.36 (m, 2H), 7.17 (tt, J = 7.3, 1.1 Hz, 1H), 7.16- 7.03 (m, 4H), 6.13 (s, 2H), 5.12 (hept, J = 7.2 Hz, 1H), 3.98 (s, 2H), 3.60-3.51 (m, 1H), 2.63-2.53 (m, 1H), 1.99-1.89 (m, 2H), 1.83-1.73 (m, 2H), 1.62 (dd, J = 8.9, 6.9 Hz, 6H), 1.54-1.36 (m, 2H), 1.33-1.19 (m, 2H). MS: 542.2 |
| (E)-1-(4-((4-amino- 7-isopropyl-5-(4- phenoxyphenyl)-7H- pyrrolo[2,3- d]pyrimidin-6- yl)ethynyl)piperidin- 1-yl)-4- (dimethylamino) but-2-en-1-one | 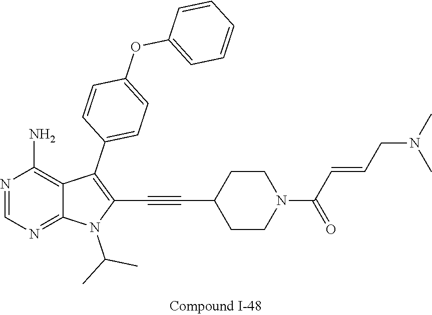 [SMILES: CC(C)n1c(C#CC2CCN(C(=O)/C=C/CN(C)C)CC2)c(-c2ccc(Oc3ccccc3)cc2)c2c(N)ncnc21] | ¹H NMR (300 MHz, CDCl₃) δ 8.31 (s, 1H), 7.48 (d, J = 8.6 Hz, 2H), 7.43-7.35 (m, 2H), 7.16 (t, J = 7.4, 7.4 Hz, 1H), 7.12-7.05 (m, 4H), 6.83 (dt, J = 15.2, 6.0, 6.0 Hz, 1H), 6.47 (d, J = 15.2 Hz, 1H), 5.21 (p, J = 7.0, 7.0, 6.9, 6.9 Hz, 1H), 5.07 (s, 2H), 3.80 (s, 1H), 3.65 (s, 1H), 3.48 (s, 2H), 3.14 (d, J = 6.1 Hz, 2H), 2.95 (dd, J = 7.7, 3.8 Hz, 1H), 2.32 (s, 6H), 1.94-1.82 (m, 2H), 1.68 (d, J = 7.0 Hz, 8H). MS: 563.3 |
| 1-(4-((4-amino-7- isopropyl-5-(4- phenoxyphenyl)-7H- pyrrolo[2,3- d]pyrimidin-6-yl) ethynyl)piperidin-1- yl)but-2-yn-1-one | 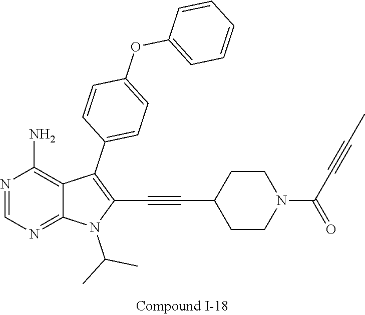 [SMILES: CC#CC(=O)N1CCC(C#Cc2c(-c3ccc(Oc4ccccc4)cc3)c3c(N)ncnc3n2C(C)C)CC1] | ¹H NMR (300 MHz, CDCl₃) δ 8.31 (s, 1H), 7.52-7.46 (m, 2H), 7.44- 7.35 (m, 2H), 7.20-7.13 (m, 1H), 7.12-7.05 (m, 4H), 5.19 (p, J = 6.9, 6.9, 6.9, 6.9 Hz, 3H), 3.80 (ddd, J = 13.6, 7.5, 3.6 Hz, 1H), 3.67 (dddd, J = 21.0, 13.5, 7.5, 3.7 Hz, 2H), 3.58- 3.47 (m, 1H), 2.96 (tt, J = 7.6, 7.6, 4.0, 4.0 Hz, 1H), 2.01 (s, 3H), 1.86 (tq, J = 11.7, 11.7, 4.0, 3.9, 3.9 Hz, 2H), 1.76 (s, 2H), 1.66 (d, J = 7.0 Hz, 6H). MS: 518.2 |
| 7-isopropyl-5-(4- phenoxyphenyl)-6- ((1- (vinylsulfonyl) piperidin-4-yl)ethynyl)- 7H-pyrrolo[2,3- d]pyrimidin-4-amine | 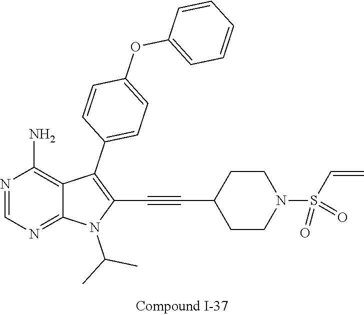 [SMILES: C=CS(=O)(=O)N1CCC(C#Cc2c(-c3ccc(Oc4ccccc4)cc3)c3c(N)ncnc3n2C(C)C)CC1] | ¹H NMR (300 MHz, DMSO) δ 8.17 (s, 1H), 7.58-7.41 (m, 4H), 7.29- 7.08 (m, 5H), 6.76 (dd, J = 16.4, 10.0 Hz, 1H), 6.19-6.04 (m, 3H), 5.17- 5.07 (m, 1H), 1.89 (s, 2H), 1.62 (d, J = 6.9 Hz, 8H). MS: 542.4 |
| 1-(6-((4-amino-7- isopropyl-5-(4- phenoxyphenyl)-7H- pyrrolo[2,3- d]pyrimidin-6- yl)ethynyl)-2- azaspiro[3.3]heptan- 2-yl)prop-2-en-1-one | 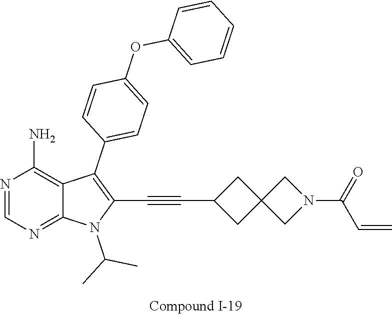 [SMILES: C=CC(=O)N1CC2(CC(C#Cc3c(-c4ccc(Oc5ccccc5)cc4)c4c(N)ncnc4n3C(C)C)C2)C1] | ¹H NMR (300 MHz, DSMO) δ 8.17 (s, 1H), 7.54-7.37 (m, 4H), 7.14- 7.11 (m, 5H), 6.30-6.03 (m, 3H), 5.64 (d, J = 8.8 Hz, 1H), 5.13-5.06 (m, 1H), 4.15 (d, J = 35.0 Hz, 2H), 3.88 (d, J = 18.8 Hz, 2H), 2.58 (d, J = 10.9 Hz, 2H), 2.27-2.24 (m, 2H), 1.61 (d, J = 6.9 Hz, 6H). MS: 518.2 |
| 1-(4-((4-amino-7-(1- methoxypropan-2- yl)-5-(4- phenoxyphenyl)-7H- pyrrolo[2,3- d]pyrimidin-6- yl)ethynyl)piperidin- 1-yl)prop-2-en-1-one | 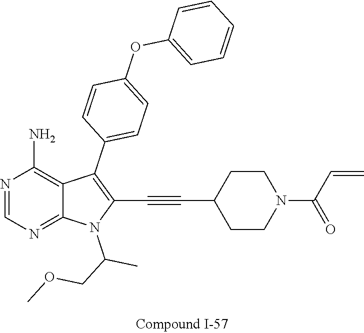 [SMILES: C=CC(=O)N1CCC(C#Cc2c(-c3ccc(Oc4ccccc4)cc3)c3c(N)ncnc3n2C(C)COC)CC1] | ¹H NMR (400 MHz, DMSO-₆) δ 8.15 (s, 1H), 7.51 (d, J = 8.2 Hz, 2H), 7.43 (t, J = 7.7 Hz, 2H), 7.18 (t, J = 7.4 Hz, 1H), 7.10 (d, J = 8.1 Hz, 4H), 6.79 (dd, J = 16.6, 10.4 Hz, 1H), 6.09 (d, J = 16.5 Hz, 1H), 5.71- 5.63 (m, 1H), 5.11 (d, J = 8.0 Hz, 1H), 4.20 (t, J = 9.6 Hz, 1H), 3.70 (dd, J = 10.3, 5.5 Hz, 3H), 3.49 (m, 2H), 3.22 (s, 3H), 3.03 (s, 1H), 1.80 (s, 2H), 1.55 (d, J = 7.1 Hz, 5H). MS: 536.25 |
| 1-(4-((4-amino-5-(4- phenoxyphenyl)-7- (tetrahydrofuran-3- yl)-7H-pyrrolo[2,3- d]pyrimidin-6- yl)ethynyl)piperidin- 1-yl)prop-2-en-1-one | 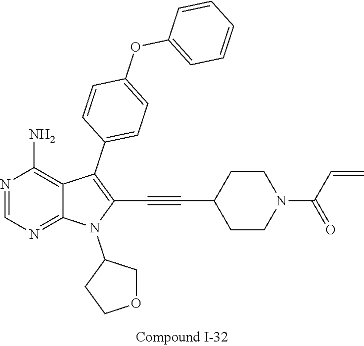 [SMILES: C=CC(=O)N1CCC(C#Cc2c(-c3ccc(Oc4ccccc4)cc3)c3c(N)ncnc3n2C2CCOC2)CC1] | ¹H NMR (400 MHz, DMSO-d₆) δ 8.19 (s, 1H), 7.51 (d, J = 8.5 Hz, 2H), 7.43 (t, J = 7.7 Hz, 2H), 7.18 (t, J = 7.3 Hz, 1H), 7.11 (d, J = 8.1 Hz, 4H), 6.80 (dd, J = 16.7, 10.5 Hz, 1H), 6.07 (dd, J = 16.8, 2.5 Hz, 1H), 5.67 (dd, J = 10.4, 2.4 Hz, 1H), 5.52 (p, J = 7.9 Hz, 1H), 4.19 (q, J = 7.8 Hz, 1H), 4.08-4.03 (m, 2H), 3.93 (q, J = 7.3 Hz, 1H), 3.65 (s, 2H), 3.44-3.35 (m, 1H), 3.03 (d, J = 4.1 Hz, 1H), 2.63 (dt, J = 14.5, 7.0 Hz, 1H), 2.36 (dt, J = 14.5, 7.0 Hz, 1H), 1.80 (s, 2H), 1.54 (s, 2H). MS: 534.2 |
| 3-(6-((1- acryloylpiperidin-4- yl)ethynyl)-4-amino- 5-(4- phenoxyphenyl)-7H- pyrrolo[2,3- d]pyrimidin-7- yl)pyrrolidin-2-one | 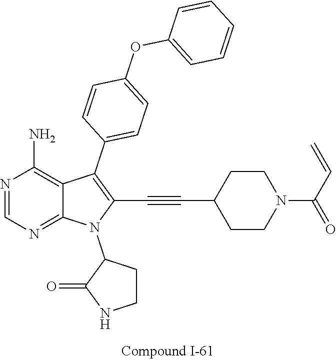 [SMILES: C=CC(=O)N1CCC(C#Cc2c(-c3ccc(Oc4ccccc4)cc3)c3c(N)ncnc3n2C2CCNC2=O)CC1] | ¹H NMR (400 MHz, DMSO-d₆) δ 8.14 (d, J = 16.8 Hz, 2H), 7.61- 7.49 (m, 2H), 7.49-7.36 (m, 2H), 7.18 (t, J = 7.4 Hz, 1H), 7.15-7.06 (m, 4H), 6.79 (dd, J = 16.7, 10.5 Hz, 1H), 6.09 (dd, J = 16.7, 2.4 Hz, 1H), 5.67 (dd, J = 10.4, 2.4 Hz, 1H), 3.67 (s, 2H), 3.41 (s, 4H), 2.99 (s, 1H), 2.67 (s, 3H), 1.78 (s, 2H), 1.53 (s, 2H). MS: 547.3 |
| 4-((4-amino-7- isopropyl-5-(4- phenoxyphenyl)-7H- pyrrolo[2,3- d]pyrimidin-6- yl)ethynyl)piperidine- 1-carbonitrile | 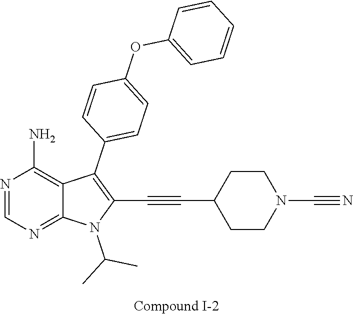 | ¹H NMR (300 MHz, DMSO-d₆) δ 8.16 (s, 1H), 7.52-7.41 (m, 4H), 7.20-7.10 (m, 5H), 6.17 (s, 2H), 5.11 (h, J = 6.7 Hz, 1H), 3.28-3.20 (m, 2H), 3.17-3.09 (m, 2H), 2.96 (tt, J = 7.8, 3.8 Hz, 1H), 1.87 (ddt, J = 13.8, 7.2, 3.7 Hz, 2H), 1.68-1.57 (m, 2H). MS: 447.25 |
| 1-(4-((4-amino-7- methyl-5-(4- phenoxyphenyl)-7H- pyrrolo[2,3- d]pyrimidin-6- yl)ethynyl)piperidin- 1-yl)prop-2-en-1-one | 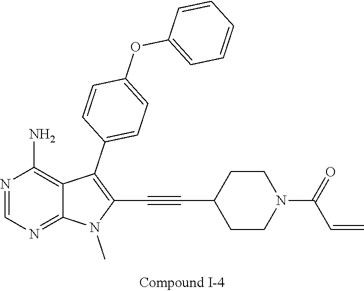 [SMILES: C=CC(=O)N1CCC(C#Cc2c(-c3ccc(Oc4ccccc4)cc3)c3c(N)ncnc3n2C)CC1] | ¹H NMR (300 MHz, DMSO) δ 8.18 (s, 1H), 7.57-7.48 (m, 2H), 7.46- 7.36 (m, 2H), 7.21-7.06 (m, 5H), 6.79 (dd, J = 16.7, 10.5 Hz, 1H), 6.21 (s, 2H), 6.09 (dd, J = 16.7, 2.5 Hz, 1H), 5.67 (dd, J = 10.4, 2.4 Hz, 1H), 3.72 (s, 3H), 3.71-3.60 (m, 2H), 3.53- 3.39 (m, 2H), 3.04 (dq, J = 7.6, 3.8 Hz, 1H), 1.81 (s, 2H), 1.57 (s, 2H). MS: 478.20 |
| 1-(3-((4-amino-7- isopropyl-5-(4- phenoxyphenyl)-7H- pyrrolo[2,3- d]pyrimidin-6- yl)ethynyl)pyrrolidin- 1-yl)prop-2-en-1-one | 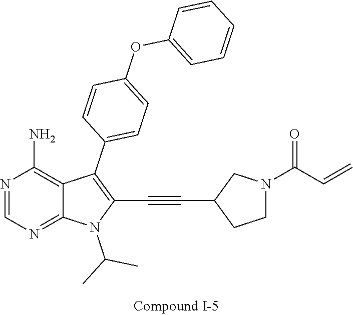 [SMILES: C=CC(=O)N1CCC(C#Cc2c(-c3ccc(Oc4ccccc4)cc3)c3c(N)ncnc3n2C(C)C)C1] | ¹H NMR (300 MHz, DMSO-d₆) δ 8.17 (s, 1H), 7.50-7.42 (m, 4H), 7.19-7.08 (m, 5H), 6.56 (ddd, J = 16.7, 10.3, 3.0 Hz, 1H), 6.14 (dt, J = 16.7, 2.9 Hz, 1H), 5.65 (ddd, J = 9.7, 7.0, 2.4 Hz, 1H), 5.10 (d, J = 2.5 Hz, 1H), 3.85 (dd, J = 10.0, 6.7 Hz, 1H), 3.64-3.59 (m, 1H), 3.47-3.44 (m, 2H), 2.21 (ddd, J = 25.0, 12.6, 6.5 Hz, 1H), 1.98 (ddd, J = 27.2, 12.6, 6.5 Hz, 1H). MS: 492.2 |
| 1-(4-((4-amino-7-(2- (4-methylpiperazin- 1-yl)ethyl)-5-(4- phenoxyphenyl)-7H- pyrrolo[2,3- d]pyrimidin-6- yl)ethynyl)piperidin- 1-yl)prop-2-en-1-one | 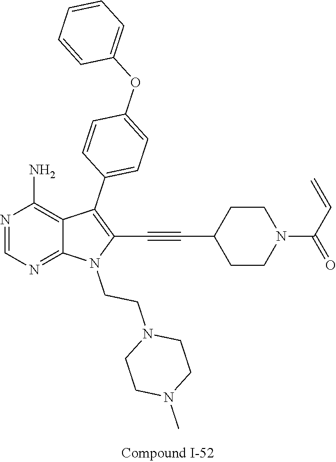 [SMILES: C=CC(=O)N1CCC(C#Cc2c(-c3ccc(Oc4ccccc4)cc3)c3c(N)ncnc3n2CCN2CCN(C)CC2)CC1] | ¹H NMR (300 MHz, DMSO-d₆) δ 8.18 (s, 1H), 7.53-7.40 (m, 4H), 7.20-7.10 (m, 5H), 6.80 (dd, J = 16.7, 10.4 Hz, 1H), 6.10 (dd, J = 16.7, 2.5 Hz, 1H), 5.67 (dd, J = 10.5, 2.5 Hz, 1H), 4.31 (t, J = 7.1 Hz, 2H), 3.68 (s, 2H), 3.43 (s, 2H), 3.04 (s, 1H), 2.68 (t, J = 7.0 Hz, 2H), 2.45 (s, 4H), 2.28 (s, 4H), 2.13 (s, 3H), 1.81 (s, 2H), 1.55 (s, 2H). MS: 590.4 |
| 1-(4-((4-amino-5-(4- phenoxyphenyl)-7- (tetrahydro-2H- pyran-4-yl)-7H- pyrrolo[2,3- d]pyrimidin-6- yl)ethynyl)piperidin- 1-yl)prop-2-en-1-one | 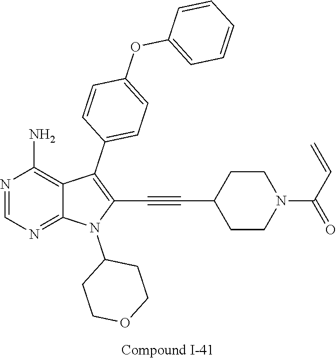 [SMILES: C=CC(=O)N1CCC(C#Cc2c(-c3ccc(Oc4ccccc4)cc3)c3c(N)ncnc3n2C2CCOCC2)CC1] | ¹H NMR (300 MHz, DMSO-d₆) δ 8.179 (s, 1H), 7.552-7.407 (m, 4H), 7.206-7.116 (m, 1H), 7.094-7.088 (m, 4H), 6.847-6.756 (dd, J = 16.7, 10.5 Hz, 1H), 6.128-6.065 (dd, J = 16.7, 2.5 Hz, 1H), 5.692-5.649 (dd, J = 10.5, 2.5 Hz, 1H), 4.948-4.906 (d, J = 12.2 Hz, 1H), 4.054-4.004 (dd, J = 11.3, 4.1 Hz, 2H), 3.698-3.662 (s, 2H), 3.501-3.433 (t, J = 11.8 Hz, 4H), 3.070-3.020 (dd, J = 7.7, 4.0 Hz, 1H), 2.816-2.708 (m, 2H), 1.804-1.713 (m, 4H), 1.549 (s, 2H). MS: 548.25 |
| 1-(4-((4-amino-5-(4- phenoxyphenyl)-7- (piperidin-4-yl)-7H- pyrrolo[2,3- d]pyrimidin-6- yl)ethynyl)piperidin- 1-yl)prop-2-en-1-one | 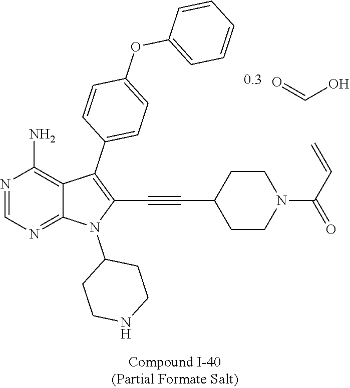 | ¹H NMR (400 MHz, DMSO-d₆) δ 8.27 (s, 0.3H), 8.18 (s, 1H), 7.51- 7.49 (m, 2H), 7.44-7.40 (m, 2H), 7.19 (t, J = 7.4 Hz, 1H), 7.11-7.08 (m, 4H), 6.80 (dd, J = 16.7, 10.5 Hz, 1H), 6.10 (dd, J = 16.7, 2.4 Hz, 1H), 5.68 (dd, J = 10.4, 2.4 Hz, 1H), 4.87 (t, J = 12.2 Hz, 1H), 3.61 (s, 2H), 3.42 (s, 2H), 3.36 (d, J = 12.0 Hz, 2H), 3.06 (tt, J = 7.7, 4.0 Hz, 1H), 2.98 (t, J = 12.6 Hz, 2H), 2.81 (q, J = 12.6 Hz, 2H), 1.90-1.88 (m, 2H), 1.81 (s, 2H), 1.57 (s, 2H). MS: 547.35 |
| 1-(4-((4-amino-5-(4- phenoxyphenyl)-7- (piperidin-3-yl)-7H- pyrrolo[2,3-d] pyrimidin-6- yl)ethynyl)piperidin- 1-yl)prop-2-en-1-one | 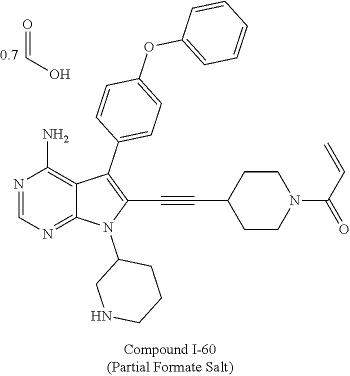 [SMILES: C=CC(=O)N1CCC(C#Cc2c(-c3ccc(Oc4ccccc4)cc3)c3c(N)ncnc3n2C2CCCNC2)CC1 ; O=CO] | ¹H NMR (400 MHz, DMSO-d₆) δ 8.21 (s, 1H), 8.16 (s, 1H), 7.50- 7.39 (m, 4H), 7.18 (t, J = 7.4 Hz, 1H), 7.10 (dd, J = 8.5, 2.3 Hz, 4H), 6.79 (dd, J = 16.7, 10.5 Hz, 1H), 6.30 (s, 1H), 6.09 (dd, J = 16.7, 2.4 Hz, 1H), 5.66 (dd, J = 10.5, 2.4 Hz, 1H), 4.78 (d, J = 11.1 Hz, 1H), 3.68 (s, 2H), 3.56 (t, J = 11.4 Hz, 1H), 3.45 (s, 3H), 3.03 (dd, J = 29.6, 7.9 Hz, 3H), 2.66 (d, J = 3.9 Hz, 1H), 1.85 (d, J = 34.1 Hz, 4H), 1.51 (s, 3H). MS: 547.30 |
| 1-(3-((4-amino-7- isopropyl-5-(4- phenoxyphenyl)-7H- pyrrolo[2,3- d]pyrimidin-6- yl)ethynyl)azetidin- 1-yl)-2-chloroethan- 1-one | 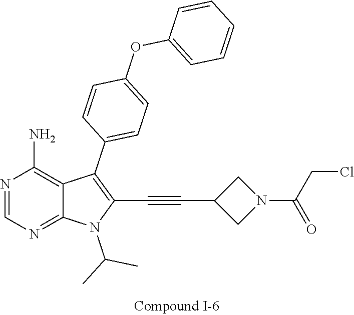 [SMILES: CC(C)n1c(C#CC2CN(C(=O)CCl)C2)c(-c2ccc(Oc3ccccc3)cc2)c2c(N)ncnc21] | ¹H NMR (400 MHz, Methanol-d₄) δ 8.15 (s, 1H), 7.53-7.50 (m, 2H), 7.42- 7.38 (m, 2H), 7.18-7.08 (m, 5H), 5.17 (p, J = 7.0 Hz, 1H), 4.60 (t, J = 8.6 Hz, 1H), 4.34 (t, J = 9.3 Hz, 1H), 4.26 (dd, J = 8.6, 6.1 Hz, 1H), 4.02- 3.95 (m, 3H), 3.81-3.77 (m, 1H), 1.71 (d, J = 7.0 Hz, 6H). MS: 500.15 |
| 1-(4-((4-amino-7- isopropyl-5-(4- phenoxyphenyl)-7H- pyrrolo[2,3- d]pyrimidin-6- yl)ethynyl)piperidin- 1-yl)-2- chloropropan-1-one | 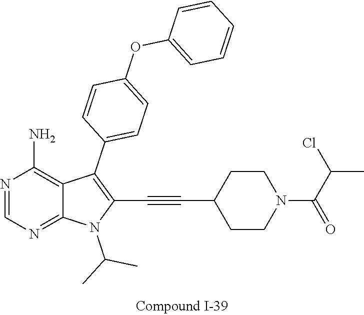 [SMILES: CC(Cl)C(=O)N1CCC(C#Cc2c(-c3ccc(Oc4ccccc4)cc3)c3c(N)ncnc3n2C(C)C)CC1] | ¹H NMR (300 MHz, Methanol-d₄) δ 8.14 (s, 1H), 7.51 (dd, J = 8.7, 2.5 Hz, 2H), 7.43-7.37 (m, 2H), 7.18- 7.05 (m, 5H), 5.18 (q, J = 6.9 Hz, 1H), 4.93 (d, J = 7.6 Hz, 1H), 3.72- 3.44 (m, 4H), 3.08 (s, 1H), 1.91 (s, 2H), 1.84-1.57 (m, 11H). MS: 542.2 |
| 1-(4-((4-amino-7- isopropyl-5-(4- phenoxyphenyl)-7H- pyrrolo[2,3- d]pyrimidin-6- yl)ethynyl)-4- hydroxypiperidin-1- yl)prop-2-en-1-one | 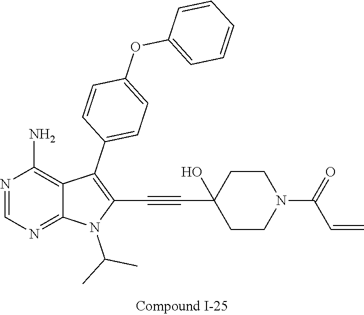 [SMILES: C=CC(=O)N1CCC(O)(C#Cc2c(-c3ccc(Oc4ccccc4)cc3)c3c(N)ncnc3n2C(C)C)CC1] | ¹H NMR (300 MHz, DMSO) δ 8.18 (s, 1H), 7.56-7.37 (m, 4H), 7.24- 7.05 (m, 5H), 6.79 (dd, J = 16.7, 10.5 Hz, 1H), 6.09 (dd, J = 16.7, 2.4 Hz, 1H), 5.87 (s, 1H), 5.67 (dd, J = 10.4, 2.4 Hz, 1H), 5.13 (p, J = 6.9 Hz, 1H), 3.91-3.71 (m, 2H), 3.28-3.12 (m, 2H), 1.89-1.79 (m, 2H), 1.70-1.56 (m, 8H). MS: 522.3 |
| 1-(4-((4-amino-7-iso propyl-5-(4-phenoxy phenyl)-7H-pyrrolo [2,3-d]pyrimidin-6- yl)ethynyl)-4- methoxypiperidin-1-yl) prop-2-en-1-one | 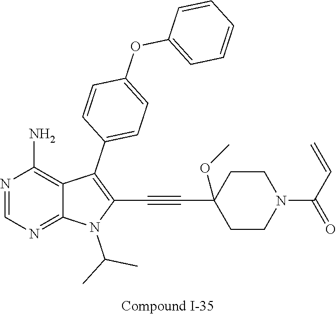 [SMILES: C=CC(=O)N1CCC(C#Cc2c(-c3ccc(Oc4ccccc4)cc3)c3c(N)ncnc3n2C(C)C)(OC)CC1] | ¹H NMR (300 MHz, CDCl₃) δ 8.34 (s, 1H), 7.55-7.46 (m, 2H), 7.45- 7.36 (m, 2H), 7.18 (t, J = 7.4, 7.4 Hz, 1H), 7.14-7.04 (m, 4H), 6.58 (dd, J = 16.8, 10.5 Hz, 1H), 6.30 (dd, J = 16.9, 2.0 Hz, 1H), 5.72 (dd, J = 10.5, 2.0 Hz, 1H), 5.39-5.07 (m, 3H), 3.93 (s, 1H), 3.71 (s, 1H), 3.45 (s, 2H), 3.37 (s, 3H), 1.96 (s, 2H), 1.90- 1.81 (m, 2H), 1.73 (d, J = 7.0 Hz, 6H). MS: 536.3 |
| 3-(6-((1- acryloylpiperidin-4- yl)ethynyl)-4-amino- 5-(4- phenoxyphenyl)-7H- pyrrolo[2,3- d]pyrimidin-7-yl)-1- methylpyrrolidin-2- one | 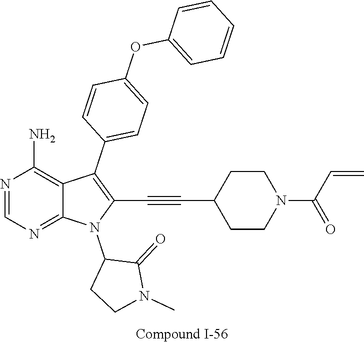 [SMILES: C=CC(=O)N1CCC(C#Cc2c(-c3ccc(Oc4ccccc4)cc3)c3c(N)ncnc3n2C2CCN(C)C2=O)CC1] | ¹H NMR (400 MHz, DMSO-d₆) δ 8.18 (br, 1H), 7.52 (d, J = 8.3 Hz, 2H), 7.48-7.38 (m, 2H), 7.18 (td, J = 7.3, 1.1 Hz, 1H), 7.18-7.07 (m, 4H), 6.80 (dd, J = 16.7, 10.5 Hz, 1H), 6.09 (dd, J = 16.7, 2.5 Hz, 1H), 5.78 (s, 1H), 5.67 (dd, J = 10.5, 2.4 Hz, 1H), 3.70 (s, 2H), 3.50 (m, 4H), 3.0 (s, 1H), 2.83 (s, 3H), 2.50 (s, 3H), 1.77 (s, 2H), 1.46 (s, 2H). MS: 561.20 |
| (E)-1-(3-((4-amino- 5-(4- phenoxyphenyl)-7- (tetrahydrofuran-3- yl)-7H-pyrrolo[2,3- d]pyrimidin-6- yl)ethynyl)azetidin- 1-yl)-4- (dimethylamino)but- 2-en-1-one | 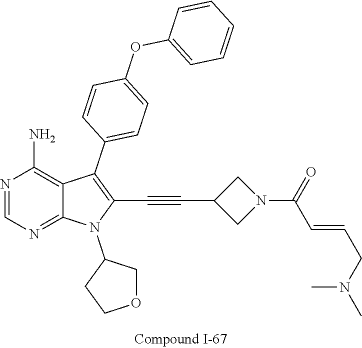 [SMILES: CN(C)C/C=C/C(=O)N1CC(C#Cc2c(-c3ccc(Oc4ccccc4)cc3)c3c(N)ncnc3n2C2CCOC2)C1] | ¹H NMR (400 MHz, DMSO-d₆) δ 8.19 (s, 1H), 7.57-7.40 (m, 4H), 7.25-7.15 (m, 1H), 7.15-7.08 (m, 4H), 6.60 (dt, J = 15.4, 6.1 Hz, 1H), 6.08 (d, J = 15.4 Hz, 1H), 5.59-5.51 (m, 1H), 4.51 (t, J = 8.4 Hz, 1H), 4.28-4.14 (m, 3H), 4.14-4.03 (m, 2H), 3.89 (q, J = 6.9, 6.0 Hz, 2H), 3.78 (q, J = 7.3, 6.7 Hz, 1H), 3.00 (dd, J = 6.1, 1.6 Hz, 2H), 2.61 (dq, J = 14.4, 7.2 Hz, 1H), 2.47-2.30 (m, 1H), 2.13 (s, 6H). MS: 563.3 |
| 1-(4-((4-amino-5-(4- (2- fluorophenoxy)phenyl)- 7- (tetrahydrofuran-3- yl)-7H-pyrrolo[2,3- d]pyrimidin-6- yl)ethynyl)piperidin- 1-yl)prop-2-en-1-one | 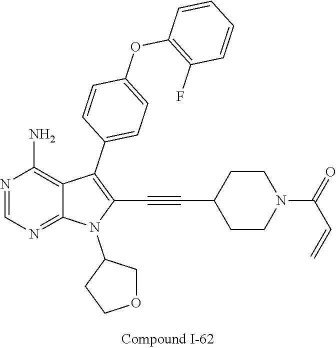 [SMILES: C=CC(=O)N1CCC(C#Cc2c(-c3ccc(Oc4ccccc4F)cc3)c3c(N)ncnc3n2C2CCOC2)CC1] | ¹H NMR (400 MHz, DMSO-d₆) δ 8.18 (s, 1H), 7.51 (d, J = 8.4 Hz, 2H), 7.44 (d, J = 8.3 Hz, 1H), 7.28 (q, J = 4.0, 3.1 Hz, 3H), 7.09 (d, J = 8.4 Hz, 2H), 6.80 (dd, J = 16.7, 10.5 Hz, 1H), 6.10 (dd, J = 16.7, 2.5 Hz, 1H), 5.67 (dd, J = 10.4, 2.4 Hz, 1H), 5.57-5.48 (m, 1H), 4.19 (q, J = 7.7 Hz, 1H), 4.14-3.88 (m, 3H), 3.72- 3.62 (m, 2H), 3.48-3.38 (m, 2H), 3.06-2.99 (m, 1H), 2.67-2.58 (m, 1H), 2.41-2.32 (m, 1H), 1.85-1.76 (m, 2H), 1.59-1.48 (m, 2H). MS: 552.2 |
| 1-(4-((4-amino-7- methyl-5-(4- phenoxyphenyl)-7H- pyrrolo[2,3- d]pyrimidin-6- yl)ethynyl)-4- hydroxypiperidin-1- yl)prop-2-en-1-one | 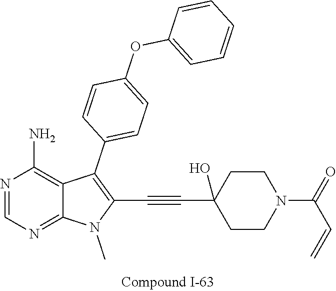 [SMILES: C=CC(=O)N1CCC(O)(C#Cc2c(-c3ccc(Oc4ccccc4)cc3)c3c(N)ncnc3n2C)CC1] | ¹H NMR (400 MHz, DMSO-d₆) δ 8.20 (s, 1H), 7.58-7.48 (m, 2H), 7.49-7.39 (m, 2H), 7.22-7.12 (m, 1H), 7.15-7.06 (m, 4H), 6.80 (dd, J = 16.7, 10.5 Hz, 1H), 6.09 (dd, J = 16.7, 2.4 Hz, 1H), 5.91 (s, 1H), 5.67 (dd, J = 10.4, 2.4 Hz, 1H), 3.94-3.89 (m, 1H), 3.83-3.76 (m, 1H), 3.75 (s, 3H), 3.31-3.28 (m, 1H), 3.19 (t, J = 10.9 Hz, 1H), 1.87 (d, J = 12.9 Hz, 2H), 1.63 (d, J = 12.5 Hz, 2H). MS: 494.2 |
| 1-(4-((4-amino-5-(4- (2- fluorophenoxy)phenyl)- 7-methyl-7H-pyrrolo [2,3-d]pyrimidin-6- yl)ethynyl)-4- hydroxypiperidin-1- yl)prop-2-en-1-one | 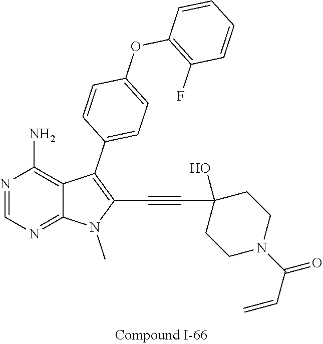 [SMILES: C=CC(=O)N1CCC(O)(C#Cc2c(-c3ccc(Oc4ccccc4F)cc3)c3c(N)ncnc3n2C)CC1] | ¹H NMR (400 MHz, DMSO-d₆) δ 8.20 (s, 1H), 7.52 (d, J = 8.6 Hz, 2H), 7.47-7.36 (m, 1H), 7.27 (q, J = 3.9, 3.1 Hz, 3H), 7.16-7.02 (m, 2H), 6.80 (dd, J = 16.7, 10.5 Hz, 1H), 6.52 (s, 1H), 6.10 (dd, J = 16.7, 2.4 Hz, 1H), 5.93 (s, 1H), 5.68 (dd, J = 10.4, 2.4 Hz, 1H), 4.00-3.82 (m, 1H), 3.74 (s, 4H), 3.30 (t, J = 8.6 Hz, 1H), 3.20 (t, J = 10.6 Hz, 1H), 1.85 (s, 2H), 1.68-1.51 (m, 2H). MS: 512.2 |
| 1-(4-((4-amino-7-(3- hydroxycyclobutyl)- 5-(4- phenoxyphenyl)-7H- pyrrolo[2,3- d]pyrimidin-6- yl)ethynyl)piperidin- 1-yl)prop-2-en-1-one | 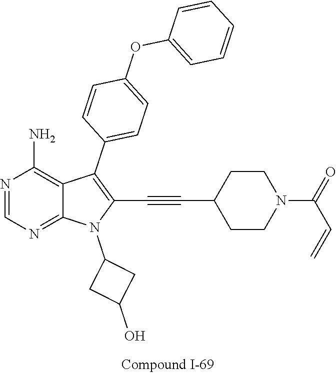 [SMILES: C=CC(=O)N1CCC(C#Cc2c(-c3ccc(Oc4ccccc4)cc3)c3c(N)ncnc3n2C2CC(O)C2)CC1] | ¹H NMR (400 MHz, DMSO-d₆) δ 8.18 (s, 1H), 7.50 (d, J = 8.2 Hz, 2H), 7.43 (t, J = 7.8 Hz, 2H), 7.18 (t, J = 7.5 Hz, 1H), 7.10 (d, J = 8.1 Hz, 4H), 6.79 (dd, J = 16.6, 10.5 Hz, 1H), 6.09 (d, J = 16.7 Hz, 1H), 5.66 (d, J = 10.4 Hz, 1H), 5.33 (d, J = 5.0 Hz, 1H), 4.90-4.76 (m, 1H), 4.04 (d, J = 7.3 Hz, 1H), 3.65 (s, 2H), 3.42 (s, 2H), 3.05 (d, J = 10.0 Hz, 2H), 2.68 (s, 2H), 1.81 (s, 2H), 1.59 (s, 2H). MS: 534.0 |
| N-(3-((4-amino-5- (4-phenoxyphenyl)- 7-(tetrahydrofuran- 3-yl)-7H- pyrrolo[2,3- d]pyrimidin-6- yl)ethynyl)cyclobutyl) acrylamide | 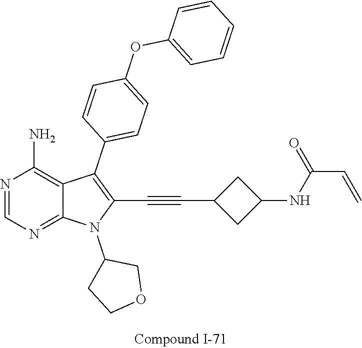 [SMILES: C=CC(=O)NC1CC(C#Cc2c(-c3ccc(Oc4ccccc4)cc3)c3c(N)ncnc3n2C2CCOC2)C1] | ¹H NMR (400 MHz, DMSO-d₆) δ 8.45 (dd, J = 13.3, 7.6 Hz, 1H), 8.19 (d, J = 2.3 Hz, 1H), 7.60-7.48 (m, 2H), 7.42 (ddd, J = 8.5, 7.3, 4.0 Hz, 2H), 7.27-7.00 (m, 5H), 6.38-6.02 (m, 3H), 5.60 (dt, J = 9.6, 2.6 Hz, 1H), 5.50 (dt, J = 17.3, 8.4 Hz, 1H), 4.55-4.16 (m, 2H), 4.12-4.05 (m, 2H), 4.00-3.85 (m, 1H), 3.25 (s, 1H), 3.00 (s, 1H), 2.68-2.53 (m, 2H), 2.33 (t, J = 8.6 Hz, 3H), 2.04 (q, J = 10.2 Hz, 1H). MS: 520.2 |
| 1-(4-((4-amino-5-(4- phenoxyphenyl)-7- (tetrahydrofuran-3- yl)-7H-pyrrolo[2,3- d]pyrimidin-6- yl)ethynyl)-4- hydroxypiperidin-1- yl)prop-2-en-1-one | 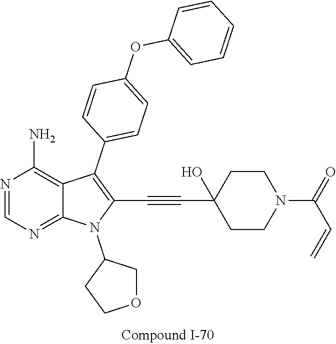 [SMILES: C=CC(=O)N1CCC(O)(C#Cc2c(-c3ccc(Oc4ccccc4)cc3)c3c(N)ncnc3n2C2CCOC2)CC1] | ¹H NMR (400 MHz, DMSO) δ 8.19 (d, J = 2.2 Hz, 1H), 7.51 (d, J = 8.2 Hz, 2H), 7.42 (t, J = 7.9 Hz, 2H), 7.17 (t, J = 7.6 Hz, 1H), 7.09 (d, J = 8.1 Hz, 4H), 6.79 (dd, J = 16.7, 10.4 Hz, 1H), 6.30 (s, 2H), 6.09 (d, J = 16.5 Hz, 1H), 5.88 (s, 1H), 5.66 (d, J = 10.5 Hz, 1H), 5.56-5.47 (m, 1H), 4.24 (d, J = 7.4 Hz, 1H), 4.07 (d, J = 8.2 Hz, 2H), 3.95 (t, J = 7.7 Hz, 1H), 3.86 (s, 1H), 3.77 (d, J = 13.6 Hz, 1H), 3.16 (s, 2H), 2.64 (dd, J = 13.1, 6.8 Hz, 1H), 2.36 (s, 2H), 1.83 (s, 2H), 1.60 (d, J = 12.3 Hz, 2H). MS: 550.25 |
| (E)-1-(3-((4-amino- 7-methyl-5-(4- phenoxyphenyl)-7H- pyrrolo[2,3- d]pyrimidin-6- yl)ethynyl)-3- hydroxyazetidin-1- yl)-4- (dimethylamino)but- 2-en-1-one | 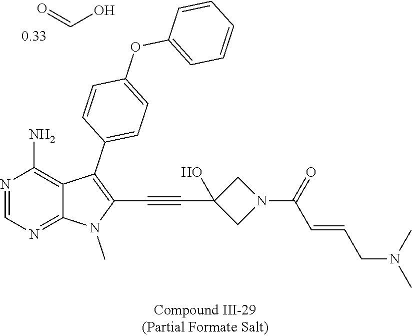 [SMILES: CN(C)C/C=C/C(=O)N1CC(O)(C#Cc2c(-c3ccc(Oc4ccccc4)cc3)c3c(N)ncnc3n2C)C1 ; O=CO] | ¹H NMR (400 MHz, DMSO-d6) δ 8.21 (s, 1H), 8.15 (s, 0.33H), 7.53 (d, J = 8.1 Hz, 2H), 7.44 (t, J = 7.7 Hz, 2H), 7.20-7.11 (m, 5H), 6.79 (s, 1H), 6.62 (dd, J = 14.6, 7.0 Hz, 1H), 6.31 (s, 1H), 6.12 (d, J = 15.3 Hz, 1H), 4.48 (d, J = 9.0 Hz, 1H), 4.26 (d, J = 9.1 Hz, 1H), 4.18 (d, J = 10.2 Hz, 1H), 3.98 (d, J = 10.2 Hz, 1H), 3.75 (s, 3H), 3.03 (d, J = 6.1 Hz, 2H), 2.15 (s, 6H). MS: 523.15 |
| (E)-1-(6-((4-amino- 7-methyl-5-(4- phenoxyphenyl)-7H- pyrrolo[2,3- d]pyrimidin-6- yl)ethynyl)-2- azaspiro[3.3]heptan- 2-yl)-4- (dimethylamino) but-2-en-1-one formate | 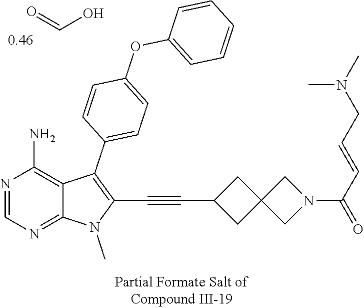 [SMILES: CN(C)C/C=C/C(=O)N1CC2(CC(C#Cc3c(-c4ccc(Oc5ccccc5)cc4)c4c(N)ncnc4n3C)C2)C1 ; O=CO] | ¹H NMR (400 MHz, DMSO-d₆) δ 8.19 (s, 1H), 8.14 (s, 0.46H), 7.51 (dd, J = 8.5, 4.3 Hz, 2H), 7.43 (t, J = 7.7 Hz, 2H), 7.15 (dt, J = 14.0, 7.9 Hz, 5H), 6.61-6.50 (m, 1H), 6.14- 6.03 (m, 1H), 4.21 (s, 1H), 4.11 (s, 1H), 3.91 (s, 1H), 3.86 (s, 1H), 3.72 (s, 3H), 3.18 (m, 3H), 3.14 (s, 2H), 2.25 (t, J = 11.7 Hz, 8H). MS: 547.25 |
| (E)-1-(6-((4-amino- 7-methyl-5-(4- phenoxyphenyl)-7H- pyrrolo[2,3- d]pyrimidin-6- yl)ethynyl)-6- hydroxy-2- azaspiro[3.3]heptan- 2-yl)-4- (dimethylamino) but-2-en-1-one | 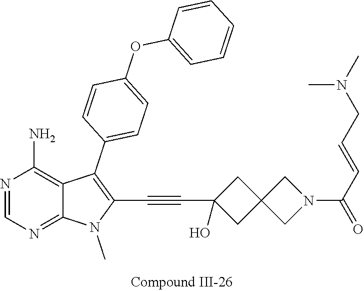 [SMILES: CN(C)C/C=C/C(=O)N1CC2(C1)CC(O)(C#Cc1c(-c3ccc(Oc4ccccc4)cc3)c3c(N)ncnc3n1C)C2] | ¹H NMR (400 MHz, DMSO-d₆) δ 8.20 (s, 1H), 7.54 (t, J = 8.0 Hz, 2H), 7.41 (t, J = 7.9 Hz, 2H), 7.23-7.03 (m, 5H), 6.53 (dt, J = 15.1, 8.5 Hz, 1H), 6.21 (s, 1H), 6.12-5.88 (m, 2H), 4.17 (s, 1H), 4.01 (s, 1H), 3.86 (d, J = 5.6 Hz, 2H), 3.75 (s, 3H), 2.97 (dd, J = 21.5, 6.1 Hz, 2H), 2.61 (d, J = 11.2 Hz, 2H), 2.42 (d, J = 10.9 Hz, 2H), 2.10 (d, J = 23.5 Hz, 6H). MS: 563.30 |
| (E)-1-(3-((4-amino- 7-methyl-5-(4- phenoxyphenyl)-7H- pyrrolo[2,3- d]pyrimidin-6- yl)ethynyl)pyrrolidin- 1-yl)-4- (dimethylamino)but- 2-en-1-one | 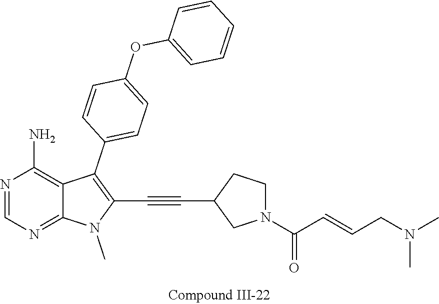 [SMILES: CN(C)C/C=C/C(=O)N1CCC(C#Cc2c(-c3ccc(Oc4ccccc4)cc3)c3c(N)ncnc3n2C)C1] | ¹H NMR (400 MHz, DMSO-d₆) δ 8.19 (s, 1H), 7.50-7.42 (m, 4H), 7.20-7.16 (m, 2H), 7.11 (dd, J = 5.7, 2.7 Hz, 3H), 6.61 (m, 1H), 6.35 (d, J = 14.3 Hz, 1H), 6.29-6.00 (br, 1H), 3.71 (s, 3H), 3.63-3.60 (m, 2H), 3.47-3.43 (m, 3H), 2.99 (d, J = 6.2 Hz, 2H), 2.30-2.14 (m, 1H), 2.13 (s, 6H), 2.12-1.92 (m, 1H). MS: 521.30 |
| 1-(3-((4-amino-7- methyl-5-(4- phenoxyphenyl)-7H- pyrrolo[2,3- d]pyrimidin-6- yl)ethynyl)pyrrolidin- 1-yl)prop-2-en-1-one | 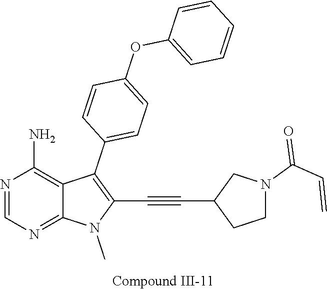 [SMILES: C=CC(=O)N1CCC(C#Cc2c(-c3ccc(Oc4ccccc4)cc3)c3c(N)ncnc3n2C)C1] | ¹H NMR (400 MHz, DMSO-d₆) δ 8.19 (s, 1H), 7.50-7.42 (m, 4H), 7.18-7.10 (m, 5H), 6.56 (ddd, J = 16.7, 10.3, 3.9 Hz, 1H), 6.42-6.28 (br, 1H), 6.14 (dt, J = 16.6, 2.6 Hz, 1H), 5.65 (ddd, J = 9.0, 6.2, 2.4 Hz, 1H), 3.87 (dd, J = 10.0, 6.9 Hz, 1H), 3.71 (s, 3H), 3.71-3.67 (m, 1H), 3.65-3.60 (m, 1H), 3.47-3.36 (m, 2H), 2.27-2.16 (m, 1H), 2.06-1.94 (m, 1H). MS: 464.20 |
| 1-(3-((4-amino-7- methyl-5-(4- phenoxyphenyl)-7H- pyrrolo[2,3- d]pyrimidin-6- yl)ethynyl)-3- hydroxypyrrolidin-1- yl)prop-2-en-1-one | 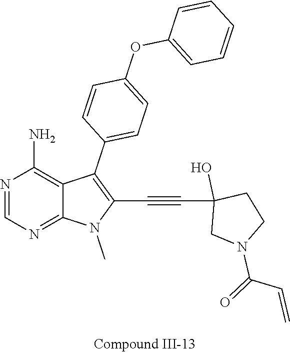 [SMILES: C=CC(=O)N1CCC(O)(C#Cc2c(-c3ccc(Oc4ccccc4)cc3)c3c(N)ncnc3n2C)C1] | ¹H NMR (400 MHz, DMSO-d₆) δ 8.21 (s, 1H), 7.55-7.48 (m, 2H), 7.48-7.39 (m, 2H), 7.19 (d, J = 7.4 Hz, 1H), 7.15-7.11 (m, 4H), 6.55 (ddd, J = 24.1, 16.7, 10.3 Hz, 1H), 6.28 (s, 1H), 6.20-6.09 (m, 2H), 5.66 (ddd, J = 16.7, 10.2, 2.4 Hz, 1H), 3.74 (s, 4H), 3.67 (dd, J = 14.4, 7.4 Hz, 1H), 3.57 (d, J = 12.4 Hz, 1H), 3.47 (dt, J = 12.0, 8.1 Hz, 0.58H), 2.24 (q, J = 6.5, 4.5 Hz, 1H), 2.21-2.10 (m, 1H). MS: 480.20 |
| (E)-1-(3-((4-amino- 7-isopropyl-5-(4- phenoxyphenyl)-7H- pyrrolo[2,3- d]pyrimidin-6- yl)ethynyl)pyrrolidin- 1-yl)-4- (dimethylamino)but- 2-en-1-one | 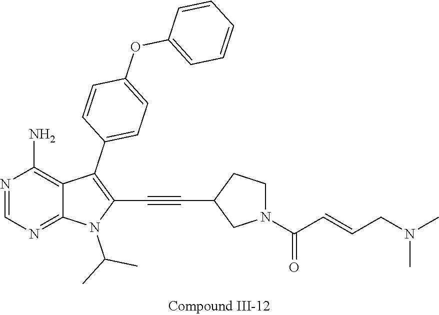 [SMILES: CC(C)n1c(C#CC2CCN(C(=O)/C=C/CN(C)C)C2)c(-c2ccc(Oc3ccccc3)cc2)c2c(N)ncnc21] | ¹H NMR (400 MHz, DMSO-d₆) δ 8.16 (s, 1H), 7.49-7.41 (m, 4H), 7.20-7.16 (t, J = 7.5 Hz, 1H), 7.14- 7.09 (td, J = 8.8, 8.4, 2.6 Hz, 4H), 6.65-6.60 (dq, J = 17.4, 6.0 Hz, 1H), 6.37-6.318 (dd, J = 15.2, 4.0 Hz, 1H), 6.10 (s, 1H), 5.12-5.08 (td, J = 6.9, 2.5 Hz, 1H), 3.83-3.81 (dd, J = 9.9, 6.8 Hz, 0.5H), 3.63-3.52 (m, 2H), 3.45-3.40 (m, 2H), 3.36- 3.34 (m, 0.5H), 3.00-2.98 (d, J = 6.2 Hz, 2H), 2.22-2.12 (m, 7H), 2.09- 2.02 (ddt, J = 43.7, 13.4, 6.5 Hz, 1H), 1.60-1.58 (d, J = 6.9 Hz, 6H). MS: 549.25 |
| 1-(3-((4-amino-7- isopropyl-5-(4- phenoxyphenyl)-7H- pyrrolo[2,3- d]pyrimidin-6- yl)ethynyl)-3- hydroxypyrrolidin-1- yl)prop-2-en-1-one | 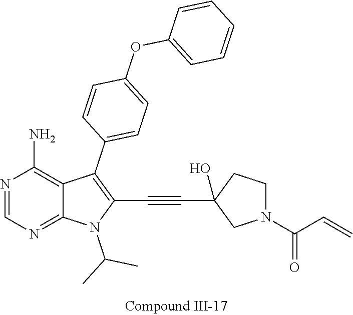 [SMILES: C=CC(=O)N1CCC(O)(C#Cc2c(-c3ccc(Oc4ccccc4)cc3)c3c(N)ncnc3n2C(C)C)C1] | ¹H NMR (400 MHz, DMSO-d₆) δ 8.18 (s, 1H), 7.52-7.41 (m, 4H), 7.20-7.10 (m, 5H), 6.59-6.48 (ddd, J = 26.6, 16.8, 10.2 Hz, 1H), 6.18- 6.09 (m, 2H), 5.70-5.62 (ddd, J = 20.0, 10.2, 2.4 Hz, 1H), 5.15-5.10 (h, J = 6.8 Hz, 1H), 3.74-3.47 (m, 4H), 2.22-2.20 (d, J = 7.8 Hz, 1H), 2.15- 2.12 (t, J = 7.2 Hz, 1H), 1.65-1.58 (d, J = 7.0 Hz, 6H). MS: 508.15 |
| 1-(3-((4-amino-5-(4- phenoxyphenyl)-7- (tetrahydrofuran-3- yl)-7H-pyrrolo[2,3- d]pyrimidin-6- yl)ethynyl)pyrrolidin- 1-yl)prop-2-en-1-one | 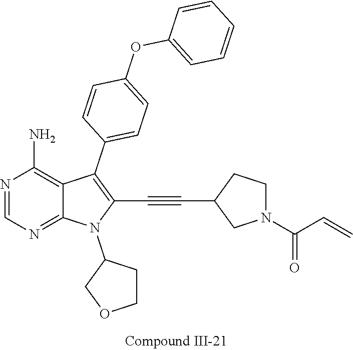 [SMILES: C=CC(=O)N1CCC(C#Cc2c(-c3ccc(Oc4ccccc4)cc3)c3c(N)ncnc3n2C2CCOC2)C1] | ¹H NMR (400 MHz, DMSO) δ 8.18 (s, 1H), 7.53-7.39 (m, 4H), 7.23- 7.06 (m, 5H), 6.55 (ddd, J = 16.7, 10.3, 1.0 Hz, 1H), 6.13 (ddd, J = 16.8, 3.6, 2.4 Hz, 1H), 5.66 (ddd, J = 10.0, 7.3, 2.4 Hz, 1H), 5.51 (s, 1H), 4.19 (t, J = 6.5, 6.5 Hz, 1H), 4.12- 4.00 (m, 2H), 3.96-3.80 (m, 2H), 3.61 (dtd, J = 21.4, 10.5, 9.9, 5.4 Hz, 2H), 3.51-3.33 (m, 2H), 2.66-2.56 (m, 1H), 2.41-2.32 (m, 1H), 2.20 (ddq, J = 32.5, 13.4, 6.8, 6.8, 6.7 Hz, 1H), 1.99 (ddt, J = 35.9, 12.3, 6.5, 6.5 Hz, 1H). MS: 520.25 |
| (E)-1-(4-((4-amino- 7-methyl-5-(4- phenoxyphenyl)-7H- pyrrolo[2,3- d]pyrimidin-6- yl)ethynyl)piperidin- 1-yl)-4- (dimethylamino)but- 2-en-1-one | 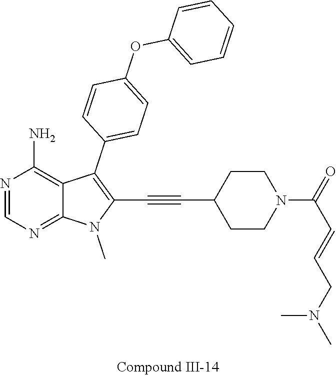 [SMILES: CN(C)C/C=C/C(=O)N1CCC(C#Cc2c(-c3ccc(Oc4ccccc4)cc3)c3c(N)ncnc3n2C)CC1] | ¹H NMR (400 MHz, DMSO-d₆) δ 8.19 (s, 1H), 7.51 (d, J = 8.5 Hz, 2H), 7.45-7.41 (m, 2H), 7.17 (t, J = 7.4 Hz, 1H), 7.11 (dd, J = 8.6, 2.8 Hz, 5H), 6.58 (s, 2H), 6.22 (s, 1H), 3.69 (s, 3H), 3.64 (d, J = 18.8 Hz, 2H), 3.45 (s, 2H), 3.03 (dd, J = 14.9, 3.5 Hz, 3H), 2.12 (s, 6H), 1.80 (s, 2H), 1.56 (s, 2H). MS: 535.35 |
| (E)-1-(4-((4-amino- 7-methyl-5-(4- phenoxyphenyl)-7H- pyrrolo[2,3- d]pyrimidin-6- yl)ethynyl)-4- hydroxypiperidin-1- yl)-4- (dimethylamino)but- 2-en-1-one | 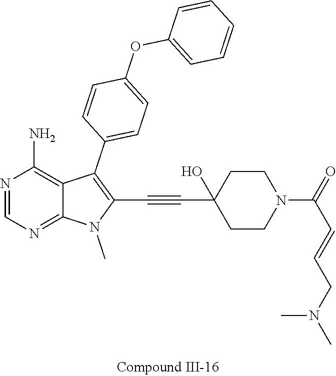 [SMILES: CN(C)C/C=C/C(=O)N1CCC(O)(C#Cc2c(-c3ccc(Oc4ccccc4)cc3)c3c(N)ncnc3n2C)CC1] | ¹H NMR (400 MHz, DMSO-d₆) δ 8.20 (s, 1H), 7.57-7.48 (m, 2H), 7.49- 7.38 (m, 2H), 7.22-7.13 (m, 1H), 7.18-7.06 (m, 4H), 6.59 (s, 2H), 6.23 (s, 2H), 5.91 (s, 1H), 3.91 (d, J = 8.5 Hz, 1H), 3.84-3.76 (m, 1H), 3.74 (s, 3H), 3.31 (d, J = 12.6 Hz, 1H), 3.16 (d, J = 12.6 Hz, 1H), 3.03-2.97 (m, 2H), 2.13 (s, 6H), 1.85 (s, 2H), 1.63 (s, 2H). MS: 551.25 |
| (E)-1-(4-((4-amino- 7-isopropyl-5-(4- phenoxyphenyl)-7H- pyrrolo[2,3- d]pyrimidin-6-yl) ethynyl)-4-hydroxy- piperidin-1-yl)-4- (dimethylamino) but-2-en-1-one | 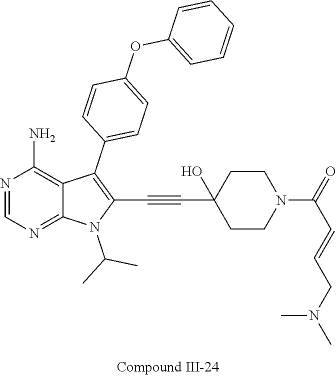 [SMILES: CC(C)n1c(C#CC2(O)CCN(C(=O)/C=C/CN(C)C)CC2)c(-c2ccc(Oc3ccccc3)cc2)c2c(N)ncnc21] | ¹H NMR (400 MHz, DMSO-d6) δ 8.17 (s, 1H), 7.51 (d, J = 8.2 Hz, 2H), 7.43 (t, J = 7.8 Hz, 2H), 7.17 (t, J = 7.3 Hz, 1H), 7.09 (d, J = 8.1 Hz, 4H), 6.58 (d, J = 2.6 Hz, 2H), 6.24 (s, 1H), 5.87 (s, 1H), 5.14 (q, J = 7.0 Hz, 1H), 3.85 (s, 1H), 3.74 (s, 1H), 3.34 (s, 1H), 3.15 (s, 1H), 3.00 (d, J = 3.4 Hz, 2H), 2.13 (s, 6H), 1.82 (s, 2H), 1.63 (d, J = 6.9 Hz, 8H). MS: 579.30 |
| (E)-1-(4-((4-amino- 5-(4- phenoxyphenyl)-7- (tetrahydrofuran-3- yl)-7H-pyrrolo[2,3- d]pyrimidin-6- yl)ethynyl)piperidin- 1-yl)-4- (dimethylamino)but- 2-en-1-one | 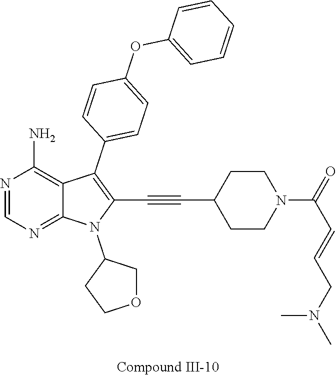 [SMILES: CN(C)C/C=C/C(=O)N1CCC(C#Cc2c(-c3ccc(Oc4ccccc4)cc3)c3c(N)ncnc3n2C2CCOC2)CC1] | ¹H NMR (400 MHz, DMSO) δ 8.19 (s, 1H), 7.51 (d, J = 8.2 Hz, 2H), 7.43 (t, J = 7.8, 7.8 Hz, 2H), 7.17 (t, J = 7.6, 7.6 Hz, 1H), 7.10 (d, J = 8.0 Hz, 4H), 6.58 (s, 2H), 5.53 (p, J = 8.3, 8.3, 8.1, 8.1 Hz, 1H), 4.19 (q, J = 7.6, 7.6, 7.4 Hz, 1H), 4.05 (q, J = 7.9, 7.9, 6.6 Hz, 2H), 3.93 (q, J = 7.7, 7.6, 7.6 Hz, 1H), 3.63 (s, 2H), 3.34 (s, 2H), 3.01 (s, 3H), 2.62 (dd, J = 12.5, 6.8 Hz, 1H), 2.35 (s, 1H), 2.14 (s, 6H), 1.79 (s, 2H), 1.53 (s, 2H). MS: 591.25 |
| (E)-1-(4-((4-amino- 5-(4- phenoxyphenyl)-7- (tetrahydrofuran-3- yl)-7H-pyrrolo[2,3- d]pyrimidin-6- yl)ethynyl)-4- hydroxypiperidin-1- yl)-4- (dimethylamino)but- 2-en-1-one | 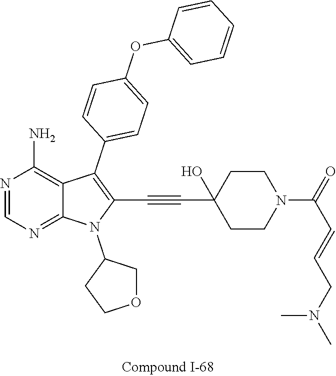 [SMILES: CN(C)C/C=C/C(=O)N1CCC(O)(C#Cc2c(-c3ccc(Oc4ccccc4)cc3)c3c(N)ncnc3n2C2CCOC2)CC1] | ¹H NMR (400 MHz, DMSO) δ 8.22 (s, 1H), 7.56-7.47 (m, 2H), 7.48- 7.37 (m, 2H), 7.22-7.12 (m, 1H), 7.14-7.05 (m, 4H), 6.59 (d, J = 2.1 Hz, 2H), 6.26 (s, 2H), 5.86 (s, 1H), 5.58-5.45 (m, 1H), 4.25 (td, J = 8.2, 5.9 Hz, 1H), 4.07 (dd, J = 8.0, 2.0 Hz, 2H), 3.94 (q, J = 7.5 Hz, 1H), 3.91- 3.81 (m, 1H), 3.80-3.70 (m, 1H), 3.33-3.21 (m, 1H), 3.20-3.10 (m, 1H), 3.06 (s, 2H), 2.64 (dt, J = 14.2, 7.3 Hz, 1H), 2.37 (td, J = 11.5, 10.3, 6.0 Hz, 1H), 2.18 (s, 6H), 1.89-1.79 (m, 2H), 1.68-1.54 (m, 2H). MS: 607.25 |
| 1-(3-((4-amino-7-(4- (dimethylamino)butyl)- 5-(4-phenoxyphenyl)- 7H-pyrrolo[2,3-d] pyrimidin-6-yl) ethynyl)pyrrolidin-1- yl)prop-2-en-1-one | 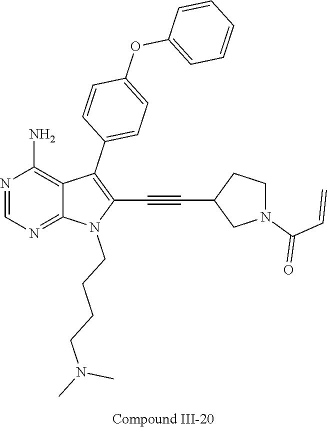 [SMILES: C=CC(=O)N1CCC(C#Cc2c(-c3ccc(Oc4ccccc4)cc3)c3c(N)ncnc3n2CCCCN(C)C)C1] | ¹H NMR (400 MHz, DMSO-d6) δ 8.19 (s, 1H), 7.59-7.39 (m, 4H), 7.28-7.04 (m, 5H), 6.56 (dd, J = 16.8, 10.3 Hz, 1H), 6.21-6.08 (m, 1H), 5.66 (t, J = 9.1 Hz, 1H), 4.21 (t, J = 7.1 Hz, 2H), 3.89-3.85 (m, 1H), 3.70-3.63 (m, 1H), 3.39-3.34 (m, 1H), 3.04-2.98 (m, 1H), 2.29-2.24 (m, 3H), 2.14 (s, 6H), 2.10-1.86 (m, 2H), 1.81-1.73 (m, 2H), 1.44-1.37 (m, 2H). MS: 549.30 |
| 1-(3-((4-amino-7-(3- (dimethylamino) propyl)-5-(4- phenoxyphenyl)-7H- pyrrolo[2,3- d]pyrimidin-6- yl)ethynyl)pyrrolidin- 1-yl)prop-2-en-1-one | 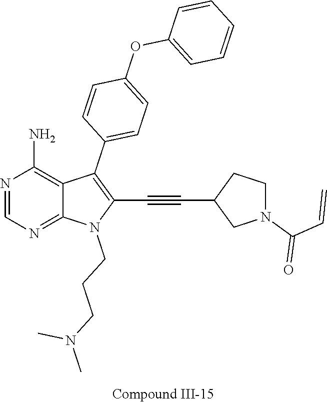 [SMILES: C=CC(=O)N1CCC(C#Cc2c(-c3ccc(Oc4ccccc4)cc3)c3c(N)ncnc3n2CCCN(C)C)C1] | ¹H NMR (400 MHz, DMSO-d₆) δ 8.18 (s, 1H), 7.50 (d, J = 8.2 Hz, 2H), 7.46-7.40 (m, 2H), 7.18 (t, J = 7.4 Hz, 1H), 7.15-7.09 (m, 4H), 6.57 (ddd, J = 16.9, 10.3, 1.8 Hz, 1H), 6.14 (dt, J = 16.8, 3.0 Hz, 1H), 5.66 (ddd, J = 9.7, 7.0, 2.3 Hz, 1H), 4.22 (t, J = 7.4 Hz, 2H), 3.87-3.60 (m, 2H), 3.58-3.36 (m, 3H), 2.29- 2.16 (m, 3H), 2.13 (s, 6H), 1.99 (ddd, J = 34.9, 12.5, 6.3 Hz, 1H), 1.86 (p, J = 7.3 Hz, 2H). MS: 535.30 |
| 1-(3-((4-amino-7-(5- (dimethylamino) pentyl)-5-(4- phenoxyphenyl)-7H- pyrrolo[2,3- d]pyrimidin-6- yl)ethynyl)pyrrolidin- 1-yl)prop-2-en-1-one | 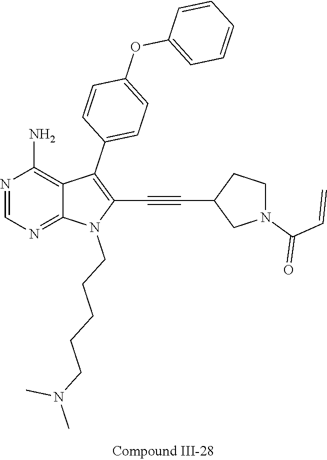 [SMILES: C=CC(=O)N1CCC(C#Cc2c(-c3ccc(Oc4ccccc4)cc3)c3c(N)ncnc3n2CCCCCN(C)C)C1] | ¹H NMR (400 MHz, DMSO-d6) δ 8.18 (s, 1H), 7.61-7.36 (m, 4H), 7.25-7.02 (m, 5H), 6.56 (dd, J = 16.8, 10.3 Hz, 1H), 6.14 (dt, J = 16.8, 2.9 Hz, 1H), 5.65 (ddd, J = 10.4, 8.1, 2.4 Hz, 1H), 4.20 (t, J = 7.2 Hz, 2H), 3.87 (dd, J = 10.1, 6.8 Hz, 0.5H), 3.73-3.41 (m, 3.5H), 3.06- 2.98 (m, 1H), 2.30-2.17 (m, 1H), 2.13 (t, J = 7.3 Hz, 2H), 2.07 (s, 6H), 2.05-1.89 (m, 1H), 1.80-1.73 (m, 2H), 1.49-1.35 (m, 2H), 1.29-1.22 (m, 2H). MS: 563.25 |
| (E)-1-(4-((4-amino- 7-methyl-5-(4- phenoxyphenyl)-7H- pyrrolo[2,3- d]pyrimidin-6- yl)ethynyl)-4- hydroxypiperidin-1- yl)-4- morpholinobut-2-en-1- one | 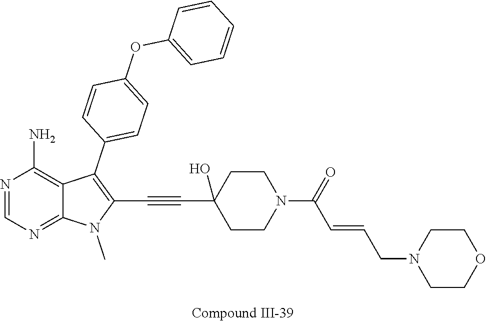 [SMILES: Cn1c(C#CC2(O)CCN(C(=O)/C=C/CN3CCOCC3)CC2)c(-c2ccc(Oc3ccccc3)cc2)c2c(N)ncnc21] | ¹H NMR (400 MHz, DMSO-d₆) δ 8.20 (s, 0H), 7.54-7.51 (m, 2H), 7.45-7.40 (m, 2H), 7.19-7.15 (m, 1H), 7.11-7.09 (m, 4H), 6.64-6.54 (m, 21H), 6.23 (br, 1H), 5.89 (s, 1H), 3.92-3.90 (d, J = 12.1 Hz, 1H), 3.75 (s, 4H), 3.58-3.57 (t, J = 4.6 Hz, 4H), 3.32 (s, 1H), 3.16-3.13 (m, J = 4.8 Hz, 1H), 3.08-3.07 (d, J = 4.8 Hz, 2H), 2.36-2.34 (t, J = 4.6 Hz, 4H), 1.85 (s, 2H), 1.63 (s, 2H). MS: 593.25 |
| (E)-1-(4-((4-amino- 7-methyl-5-(4- phenoxyphenyl)-7H- pyrrolo[2,3- d]pyrimidin-6- yl)ethynyl)-4- hydroxypiperidin-1- yl)-4-(azetidin-1- yl)but-2-en-1-one | 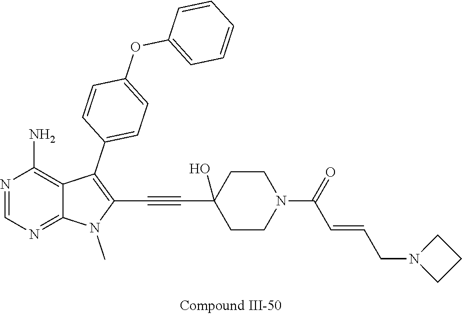 [SMILES: Cn1c(C#CC2(O)CCN(C(=O)/C=C/CN3CCC3)CC2)c(-c2ccc(Oc3ccccc3)cc2)c2c(N)ncnc21] | ¹H NMR (400 MHz, DMSO-d₆) δ 8.20 (s, 1H), 7.53-7.51 (m, 2H), 7.45-7.41 (t, J = 7.6 Hz, 2H), 7.20- 7.16 (d, J = 7.9 Hz, 1H), 7.12-7.10 (m, 4H), 6.49 (s, 2H), 5.89 (s, 1H), 3.91-3.86 (s, 1H), 3.79-3.75 (s, 4H), 3.30 (s, 1H), 3.13-3.10 (m, 7H), 1.97-1.93 (m, 2H), 1.85 (s, 2H), 1.61 (s, 2H). MS: 563.25 |
| 1-(4-((4-amino-7- methyl-5-(4- phenoxyphenyl)-7H- pyrrolo[2,3- d]pyrimidin-6- yl)ethynyl)-4- hydroxypiperidin-1- yl)-2-methylprop-2- en-1-one | 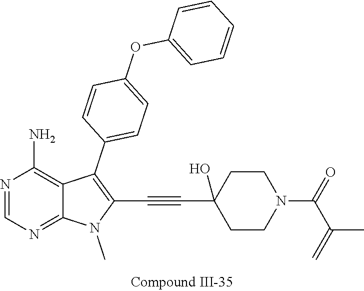 [SMILES: C=C(C)C(=O)N1CCC(O)(C#Cc2c(-c3ccc(Oc4ccccc4)cc3)c3c(N)ncnc3n2C)CC1] | ¹H NMR (400 MHz, DMSO-d₆) δ 8.20 (d, J = 1.9 Hz, 1H), 7.54-7.52 (d, J = 8.1 Hz, 2H), 7.48-7.41 (t, J = 7.8 Hz, 2H), 7.20-7.17 (d, J = 7.8 Hz, 1H), 7.12-7.10 (m, 4H), 6.46- 6.07 (br, 1H), 5.92 (d, J = 2.0 Hz, 1H), 5.15 (s, 1H), 4.96 (s, 1H), 3.92- 3.57 (t, J = 1.7 Hz, 5H), 3.21-3.20 (s, 2H), 1.88-1.83 (s, 5H), 1.66-1.63 (m, 2H). MS: 508.15 |
| 1-(4-((4-amino-7- methyl-5-(4- phenoxyphenyl)-7H- pyrrolo[2,3- d]pyrimidin-6- yl)ethynyl)piperidin- 1-yl)-2-methylprop- 2-en-1-one | 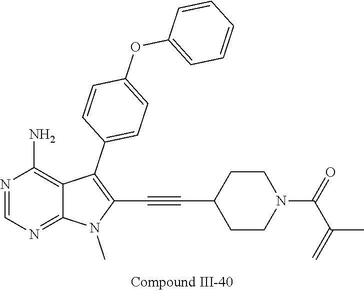 | ¹H NMR (400 MHz, DMSO-d₆) δ 8.19 (s, 1H), 7.51 (d, J = 8.1 Hz, 2H), 7.43 (t, J = 7.7 Hz, 2H), 7.18 (t, J = 7.5 Hz, 1H), 7.12-7.10 (m, 4H), 6.18 (s, 2H), 5.14 (s, 1H), 4.93 (s, 1H), 3.93 (s, 3H), 3.73 (s, 2H), 3.36 (s, 2H), 3.05 (s, 1H), 2.08 (d, J = 1.6 Hz, 1H), 1.84 (s, 5H), 1.57 (d, J = 10.9 Hz, 2H). MS: 429.25 |
| (E)-1-(4-((4-amino- 7-methyl-5-(4- phenoxyphenyl)-7H- pyrrolo[2,3- d]pyrimidin-6- yl)ethynyl)piperidin- 1-yl)-4- morpholinobut-2-en- 1-one | 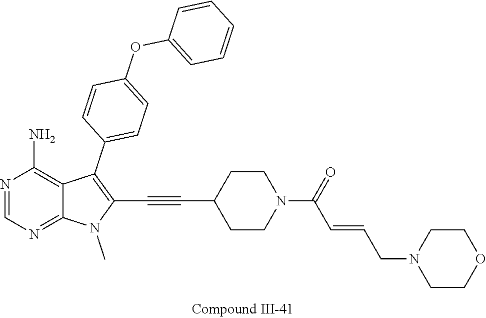 [SMILES: Cn1c(C#CC2CCN(C(=O)/C=C/CN3CCOCC3)CC2)c(-c2ccc(Oc3ccccc3)cc2)c2c(N)ncnc21] | ¹H NMR (400 MHz, DMSO-d₆) δ 8.19 (s, 1H), 7.53-7.51 (m, 2H), 7.45- 7.41 (m, 2H), 7.20-7.16 (m, 1H), 7.12-7.10 (m, 4H), 6.66-6.55 (m, 2H), 6.21 (s, 1H), 3.80 (s, 3H), 3.50 (d, 8H), 3.21-3.04 (m, 3H), 2.67 (s, 2H), 2.42 (d, 2H), 1.81 (s, 2H), 1.57 (s, 2H). MS: 577.30 |
| 1-((2S,4S)-4-((4- amino-7-methyl-5- (4-phenoxyphenyl)- 7H-pyrrolo[2,3- d]pyrimidin-6- yl)ethynyl)-2- methylpiperidin-1- yl)prop-2-en-1-one | 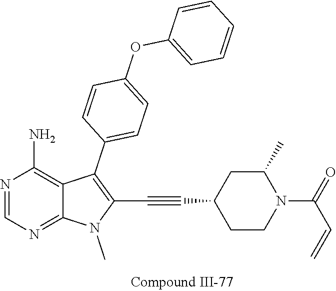 [SMILES: C=CC(=O)N1CC[C@H](C#Cc2c(-c3ccc(Oc4ccccc4)cc3)c3c(N)ncnc3n2C)C[C@@H]1C] | ¹H NMR (400 MHz, DMSO-d₆) δ 8.19 (s, 1H), 7.50 (d, J = 8.1 Hz, 2H), 7.43 (t, J = 7.6 Hz, 2H), 7.18 (t, J = 7.4 Hz, 1H), 7.12 (d, J = 8.1 Hz, 4H), 6.77 (dd, J = 16.9, 10.5 Hz, 1H), 6.26-6.12 (br, 1H), 6.07 (d, J = 16.8 Hz, 1H), 5.65 (d, J = 10.2 Hz, 1H), 4.40 (s, 1H), 3.71 (s, 3H), 3.08 (t, J = 12.4 Hz, 1H), 2.42 (s, 2H), 1.97 (d, J = 13.2 Hz, 1H), 1.87 (d, J = 13.4 Hz, 1H), 1.65 (s, 1H), 1.40 (s, 1H), 1.25 (s, 3H). MS: 492.25 |
| 1-((2S,4R)-4-((4- amino-7-methyl-5- (4-phenoxyphenyl)- 7H-pyrrolo[2,3- d]pyrimidin-6- yl)ethynyl)-2- methylpiperidin-1- yl)prop-2-en-1-one | 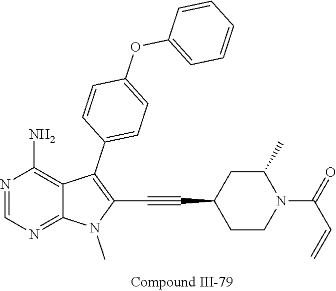 [SMILES: C=CC(=O)N1CC[C@@H](C#Cc2c(-c3ccc(Oc4ccccc4)cc3)c3c(N)ncnc3n2C)C[C@@H]1C] | ¹H NMR (400 MHz, DMSO-d₆) δ 8.19 (s, 1H), 7.50 (d, J = 8.2 Hz, 2H), 7.43 (t, J = 7.8 Hz, 2H), 7.16 (dd, J = 25.8, 7.7 Hz, 5H), 6.77 (dd, J = 16.8, 10.5 Hz, 1H), 6.20 (br, 1H), 6.07 (dd, J = 16.7, 2.3 Hz, 1H), 5.65 (dd, J = 10.5, 2.4 Hz, 1H), 4.40 (s, 1H), 3.71 (s, 3H), 3.06 (t, J = 12.5 Hz, 1H), 2.56 (s, 2H), 1.97 (d, J = 13.2 Hz, 1H), 1.87 (d, J = 13.4 Hz, 1H), 1.65 (s, 1H), 1.40 (s, 1H), 1.25 (s, 3H). MS: 492.25 |
| (R,E)-1-(4-((4- amino-5-(4- phenoxyphenyl)-7- (tetrahydrofuran-3- yl)-7H-pyrrolo[2,3- d]pyrimidin-6- yl)ethynyl) piperidin-1-yl)-4- morpholinobut-2-en- 1-one | 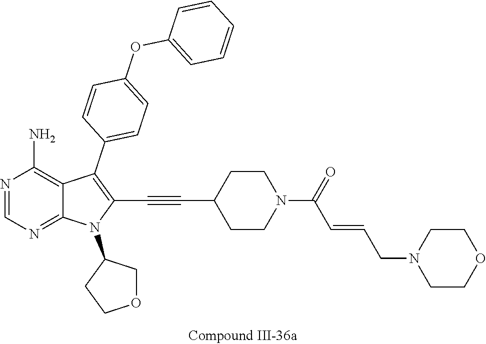 [SMILES: Nc1ncnc2c1c(-c1ccc(Oc3ccccc3)cc1)c(C#CC1CCN(C(=O)/C=C/CN3CCOCC3)CC1)n2[C@@H]1CCOC1] | ¹H NMR (400 MHz, DMSO-d₆) δ 8.18 (s, 1H), 7.55-7.47 (m, 2H), 7.47-7.38 (m, 2H), 7.17 (tt, J = 7.4, 1.2 Hz, 1H), 7.15-7.06 (m, 4H), 6.66-6.51 (m, 2H), 6.20 (s, 1H), 5.53 (p, J = 7.8 Hz, 1H), 4.25-4.14 (m, 1H), 4.14-4.01 (m, 2H), 3.93 (q, J = 7.3 Hz, 1H), 3.63 (s, 2H), 3.57 (d, J = 4.7 Hz, 4H), 3.40 (s, 2H), 3.08 (d, J = 4.6 Hz, 2H), 3.02 (dd, J = 7.9, 4.0 Hz, 1H), 2.63 (dq, J = 14.5, 7.4 Hz, 1H), 2.35 (t, J = 4.8 Hz, 5H), 1.79 (s, 2H), 1.54 (s, 2H). MS: 633.25 |
| (S,E)-1-(4-((4- amino-5-(4- phenoxyphenyl)-7- (tetrahydrofuran-3- yl)-7H-pyrrolo[2,3- d]pyrimidin-6- yl)ethynyl)piperidin- 1-yl)-4- morpholinobut-2-en- 1-one | 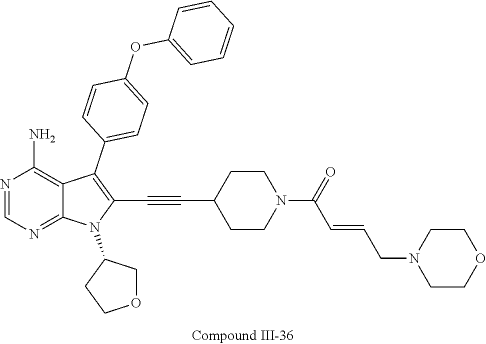 [SMILES: Nc1ncnc2c1c(-c1ccc(Oc3ccccc3)cc1)c(C#CC1CCN(C(=O)/C=C/CN3CCOCC3)CC1)n2[C@H]1CCOC1] | ¹H NMR (400 MHz, DMSO-d₆) δ 8.19 (s, 1H), 7.51 (d, J = 8.2 Hz, 2H), 7.43 (t, J = 7.8 Hz, 2H), 7.18 (d, J = 7.3 Hz, 1H), 7.10 (d, J = 8.1 Hz, 4H), 6.60 (s, 2H), 6.49-5.88 (br, 1H), 5.57-5.49 (m, 1H), 4.20 (d, J = 7.4 Hz, 1H), 4.07 (p, J = 8.6 Hz, 2H), 3.93 (q, J = 7.3 Hz, 1H), 3.63 (s, 2H), 3.57 (d, J = 5.2 Hz, 4H), 3.32 (s, 2H), 3.08-3.00 (m, 3H), 2.63 (d, J = 9.3 Hz, 1H), 2.35 (s, 5H), 1.79 (s, 2H), 1.53 (s, 2H). MS: 633.30 |
| (S)-1-(4-((4-amino- 5-(4- phenoxyphenyl)-7- (tetrahydrofuran-3- yl)-7H-pyrrolo[2,3- d]pyrimidin-6- yl)ethynyl) piperidin-1-yl)-2- methylprop-2-en-1- one | 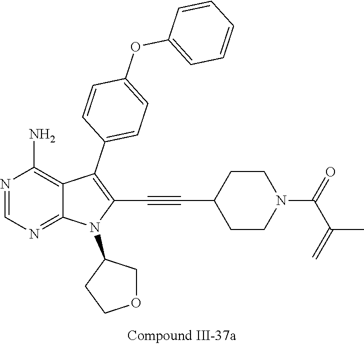 [SMILES: C=C(C)C(=O)N1CCC(C#Cc2c(-c3ccc(Oc4ccccc4)cc3)c3c(N)ncnc3n2[C@@H]2CCOC2)CC1] | ¹H NMR (400 MHz, DMSO-d₆) δ 8.19 (s, 1H), 7.52 (d, J = 8.2 Hz, 2H), 7.43 (t, J = 7.9 Hz, 2H), 7.19 (d, J = 7.4 Hz, 1H), 7.11 (dd, J = 8.4, 3.3 Hz, 4H), 6.23 (s, 1H), 5.57-5.48 (m, 1H), 5.14 (s, 1H), 4.94 (s, 1H), 4.20 (q, J = 7.9 Hz, 1H), 4.14-4.01 (m, 2H), 3.93 (q, J = 7.4 Hz, 1H), 3.56 (s, 2H), 3.35 (s, 2H), 3.03 (s, 1H), 2.63 (dd, J = 12.3, 6.6 Hz, 1H), 2.36 (s, 1H), 1.81 (d, J = 18.1 Hz, 5H), 1.54 (d, J = 10.8 Hz, 2H). MS: 548.20 |
| (S)-1-(4-((4-amino- 5-(4- phenoxyphenyl)-7- (tetrahydrofuran-3- yl)-7H-pyrrolo[2,3- d]pyrimidin-6- yl)ethynyl)piperidin- 1-yl)-2-methylprop- 2-en-1-one | 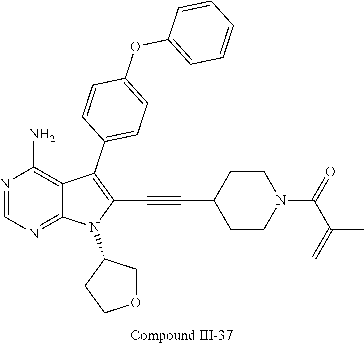 [SMILES: C=C(C)C(=O)N1CCC(C#Cc2c(-c3ccc(Oc4ccccc4)cc3)c3c(N)ncnc3n2[C@H]2CCOC2)CC1] | ¹H NMR (400 MHz, DMSO-d₆) δ 8.19 (s, 1H), 7.52 (d, J = 8.2 Hz, 2H), 7.43 (t, J = 7.8 Hz, 2H), 7.18 (t, J = 7.5 Hz, 1H), 7.11 (t, J = 5.1 Hz, 4H), 6.27-6.14 (br, 1H), 5.53 (t, J = 8.5 Hz, 1H), 5.14 (s, 1H), 4.94 (s, 1H), 4.21-4.17 (m, 1H), 4.07 (p, J = 8.9 Hz, 2H), 3.96-3.90 (m, 1H), 3.57 (s, 2H), 3.36 (d, J = 9.7 Hz, 2H), 3.03 (s, 1H), 2.62 (s, 1H), 2.36 (s, 1H), 1.81 (d, J = 18.0 Hz, 5H), 1.54 (d, J = 10.7 Hz, 2H). MS: 548.20 |
| 1-(6-[2-[4-amino-7- methyl-5-(4- phenoxyphenyl)-7H- pyrrolo[2,3- d]pyrimidin-6- yl]ethynyl]-3- azabicyclo[3.1.0] hexan-3-yl)prop-2-en- 1-one | 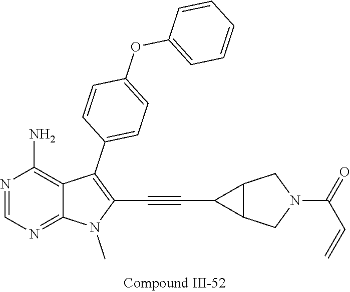 [SMILES: C=CC(=O)N1CC2C(C#Cc3c(-c4ccc(Oc5ccccc5)cc4)c4c(N)ncnc4n3C)C2C1] | ¹H NMR (400 MHz, DMSO-d6) δ 8.18 (s, 1H), 7.50-7.42 (m, 4H), 7.21-7.10 (m, 5H), 6.53-6.47 (dd, J = 16.7, 10.4 Hz, 1H), 6.13-6.09 (m, 1H), 5.67-5.64 (m, 1H), 3.90- 3.87 (d, J = 10.9 Hz, 1H), 3.77-3.73 (d, J = 12.5 Hz, 1H), 3.70 (s, 3H), 3.66-3.62 (dd, J = 10.9, 4.2 Hz, 1H), 3.42-3.38 (dd, J = 12.5, 4.3 Hz, 1H), 2.11-2.10 (m, 1H), 2.06- 2.03 (m, 1H), 1.48 (t, J = 3.5 Hz, 1H). MS: 476.10 |
| N-((1s,4s)-4-((4- amino-7-methyl-5- (4-phenoxyphenyl)- 7H-pyrrolo[2,3- d]pyrimidin-6- yl)ethynyl)cyclohexyl) acrylamide | 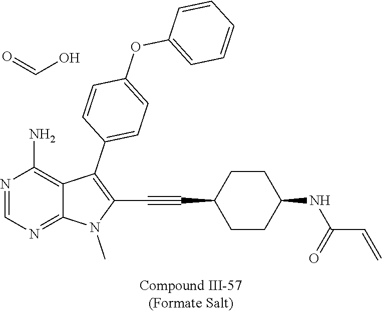 | ¹H NMR (400 MHz, DMSO-d₆) δ 8.18 (s, 1H), 7.98 (d, J = 7.7 Hz, 1H), 7.51 (d, J = 8.3 Hz, 2H), 7.43 (t, J = 7.8 Hz, 2H), 7.16 (dd, J = 22.9, 7.9 Hz, 5H), 6.21 (dd, J = 17.1, 10.0 Hz, 2H), 6.07 (d, J = 16.9 Hz, 1H), 5.56 (d, J = 10.1 Hz, 1H), 3.71 (s, 3H), 3.65 (s, 1H), 2.60 (s, 1H), 1.98 (d, J = 13.1 Hz, 2H), 1.82 (d, J = 10.4 Hz, 2H), 1.47 (q, J = 12.3 Hz, 2H), 1.33-1.22 (m, 2H). MS: 492.20 |
| N-((1r,4r)-4-((4- amino-7-methyl-5- (4-phenoxy phenyl)- 7H-pyrrolo[2,3- d]pyrimidin-6- yl)ethynyl) cyclohexyl) acrylamide | 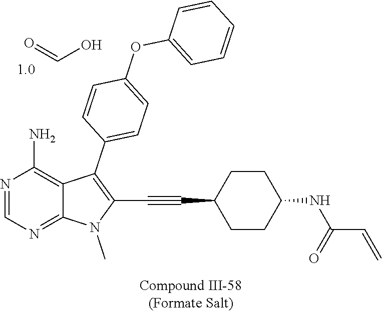 | ¹H NMR (400 MHz, DMSO-d₆) δ 8.18 (s, 1H), 7.98 (d, J = 7.7 Hz, 1H), 7.56-7.47 (m, 2H), 7.48-7.38 (m, 2H), 7.22-7.09 (m, 5H), 6.22 (dd, J = 17.1, 10.1 Hz, 2H), 6.07 (dd, J = 17.1, 2.4 Hz, 1H), 5.56 (dd, J = 10.0, 2.4 Hz, 1H), 3.71 (s, 3H), 3.66- 3.57 (m, 1H), 2.60 (t, J = 10.8 Hz, 1H), 2.02-1.93 (m, 2H), 1.87-1.78 (m, 2H), 1.47 (q, J = 12.0 Hz, 2H), 1.33-1.19 (m, 2H). MS: 492.20 |
| (R,E)-1-(3-((4- amino-5-(4- phenoxyphenyl)-7- (tetrahydrofuran-3- yl)-7H-pyrrolo[2,3- d]pyrimidin-6- yl)ethynyl)azetidin- 1-yl)-4- (dimethylamino)but- 2-en-1-one | 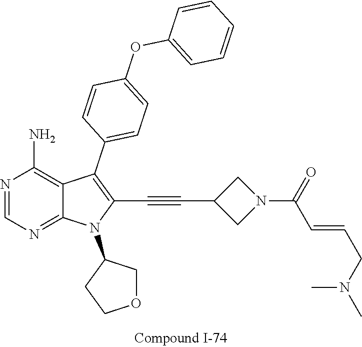 [SMILES: CN(C)C/C=C/C(=O)N1CC(C#Cc2c(-c3ccc(Oc4ccccc4)cc3)c3c(N)ncnc3n2[C@@H]2CCOC2)C1] | ¹H NMR (400 MHz, DMSO-d6) δ 8.20 (s, 1H), 7.53 (d, J = 8.3 Hz, 2H), 7.44 (t, J = 7.7 Hz, 2H), 7.20- 7.11 (m, 5H), 6.68-6.55 (m, 1H), 6.08 (d, J = 15.4 Hz, 1H), 5.60-5.51 (m, 1H), 4.51 (t, J = 8.5 Hz, 1H), 4.25-4.14 (m, 3H), 4.12-4.00 (m, 2H), 3.96-3.84 (m, 2H), 3.82-3.76 (m, 1H), 3.00 (d, J = 6.1 Hz, 2H), 2.68-2.58 (m, 1H), 2.41-2.33 (s, 1H), 2.14 (s, 6H). MS: 563.25 |
| (S,E)-1-(3-((4- amino-5-(4- phenoxyphenyl)-7- (tetrahydrofuran-3- yl)-7H-pyrrolo[2,3- d]pyrimidin-6- yl)ethynyl)azetidin- 1-yl)-4- (dimethylamino)but- 2-en-1-one | 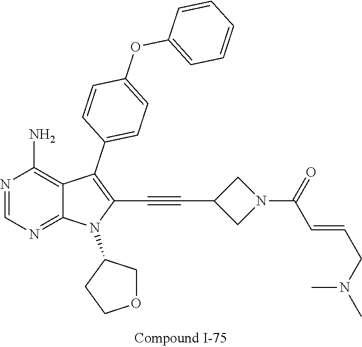 [SMILES: CN(C)C/C=C/C(=O)N1CC(C#Cc2c(-c3ccc(Oc4ccccc4)cc3)c3c(N)ncnc3n2[C@H]2CCOC2)C1] | ¹H NMR (400 MHz, DMSO-d6) δ 8.20 (s, 1H), 7.53 (d, J = 8.3 Hz, 2H), 7.44 (t, J = 7.7 Hz, 2H), 7.20- 7.11 (m, 5H), 6.68-6.55 (m, 1H), 6.08 (d, J = 15.4 Hz, 1H), 5.60-5.51 (m, 1H), 4.51 (t, J = 8.5 Hz, 1H), 4.25-4.14 (m, 3H), 4.12-4.00 (m, 2H), 3.96-3.84 (m, 2H), 3.82-3.76 (m, 1H), 3.00 (d, J = 6.1 Hz, 2H), 2.68-2.58 (m, 1H), 2.41-2.33 (s, 1H), 2.14 (s, 6H). MS: 563.25 |
| (E)-1-(3-((4-amino- 7-methyl-5-(4- phenoxyphenyl)-7H- pyrrolo[2,3- d]pyrimidin-6- yl)ethynyl)azetidin- 1-yl)-4- morpholinobut-2-en-1- one | 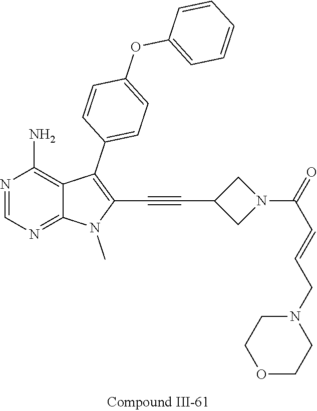 [SMILES: Cn1c(C#CC2CN(C(=O)/C=C/CN3CCOCC3)C2)c(-c2ccc(Oc3ccccc3)cc2)c2c(N)ncnc21] | ¹H NMR (400 MHz, DMSO-d₆) δ 8.20 (s, 1H), 7.54-7.52 (m, 2H), 7.46-7.42 (m, 2H), 7.20-7.16 (m, 1H), 7.14-7.11 (m, 4H), 6.61-6.57 (dt, J = 15.4, 6.3 Hz, 1H), 6.40-6.20 (br, 1H), 6.12-6.08 (dd, J = 15.4, 1.6 Hz, 1H), 4.53 (t, J = 8.4 Hz, 1H), 4.24- 4.19 (dt, J = 17.2, 8.2 Hz, 2H), 3.88- 3.86 (m, 1H), 3.80 (m, 1H), 3.75 (s, 3H), 3.57 (t, J = 4.6 Hz, 4H), 3.07 (dd, J = 6.2, 1.5 Hz, 2H), 2.35 (t, J = 4.7 Hz, 4H). MS: 549.25 |
| (E)-1-(3-((4-amino- 7-methyl-5-(4- phenoxyphenyl)-7H- pyrrolo[2,3- d]pyrimidin-6- yl)ethynyl)azetidin- 1-yl)-4-(azetidin-1- yl)but-2-en-1-one | 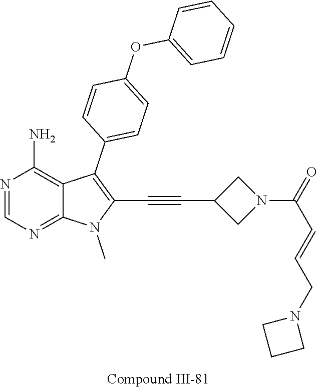 [SMILES: Cn1c(C#CC2CN(C(=O)/C=C/CN3CCC3)C2)c(-c2ccc(Oc3ccccc3)cc2)c2c(N)ncnc21] | ¹H NMR (400 MHz, DMSO-d₆) δ 8.21-8.19 (d, J = 8.2 Hz, 1H), 7.53- 7.51 (m, 2H), 7.45-7.41 (dd, J = 8.5, 7.3 Hz, 2H), 7.20-7.18 (m, 1H), 7.16-7.10 (m, 4H), 6.53-6.47 (m, 1H), 6.09-5.98 (dd, J = 31.4, 15.4 Hz, 1H), 4.54-4.50 (t, J = 8.5 Hz, 1H), 4.25-4.16 (dt, J = 15.2, 7.7 Hz, 2H), 3.89-3.85 (m, 1H), 3.83- 3.77 (m, 1H), 3.74 (s, 3H), 3.22 (s, 1H), 3.13-3.10 (dd, J = 8.5, 5.5 Hz, 4H), 3.00 (d, J = 6.0 Hz, 1H), 2.13 (s, 1H), 1.99-1.93 (p, J = 7.1 Hz, 1H). MS: 519.20 |
| (E)-1-(3-((4-amino- 7-methyl-5-(4- phenoxyphenyl)-7H- pyrrolo[2,3- d]pyrimidin-6- yl)ethynyl)azetidin- 1-yl)-4-(3- hydroxyazetidin-1- yl)but-2-en-1-one | 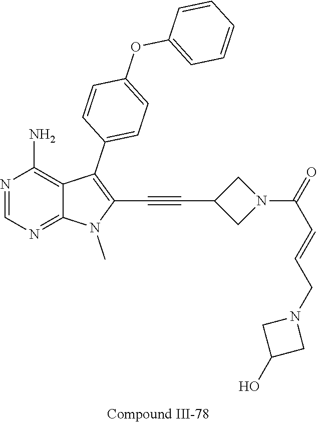 [SMILES: Cn1c(C#CC2CN(C(=O)/C=C/CN3CC(O)C3)C2)c(-c2ccc(Oc3ccccc3)cc2)c2c(N)ncnc21] | ¹H NMR (400 MHz, DMSO-d₆) δ 8.19 (s, 1H), 7.53-7.51 (m 2H), 7.45-7.41 (m, 2H), 7.20-7.16 (m, 1H), 7.14-7.10 (m, 4H), 6.52- 6.49 (dt, J = 15.4, 5.2 Hz, 1H), 6.01- 5.97 (dt, J = 15.5, 1.8 Hz, 1H), 5.30- 5.28 (d, J = 6.5 Hz, 1H), 4.52-4.49 (t, J = 8.4 Hz, 1H), 4.23-4.15 (m, 3H), 3.89-3.77 (m, 2H), 3.74 (s, 3H), 3.54-3.50 (td, J = 6.1, 2.1 Hz, 2H), 3.14-3.12 (dd, J = 5.2, 1.8 Hz, 2H), 2.72-2.69 (dd, J = 7.9, 6.0 Hz, 2H). MS: 535.25 |
| (E)-1-(6-((4-amino- 7-methyl-5-(4- phenoxyphenyl)-7H- pyrrolo[2,3- d]pyrimidin-6- yl)ethynyl)-2- azaspiro[3.3]heptan- 2-yl)-4- morpholinobut-2-en- 1-one | 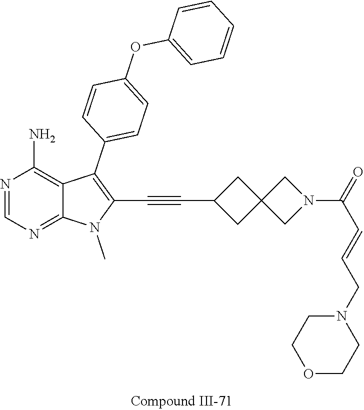 [SMILES: Cn1c(C#CC2CC3(C2)CN(C(=O)/C=C/CN2CCOCC2)C3)c(-c2ccc(Oc3ccccc3)cc2)c2c(N)ncnc21] | ¹H NMR (400 MHz, DMSO-d6) δ 8.18 (s, 1H), 7.51 (t, J = 6.9 Hz, 2H), 7.42 (t, J = 7.8 Hz, 2H), 7.14 (dq, J = 13.7, 7.2 Hz, 5H), 6.53 (dq, J = 12.8, 6.1 Hz, 1H), 6.23 (br, 1H), 6.09- 6.00 (m, 1H), 4.20 (s, 1H), 4.10 (s, 1H), 3.87 (d, J = 16.5 Hz, 2H), 3.71 (s, 3H), 3.56 (p, J = 4.3 Hz, 4H), 3.20 (p, J = 7.9 Hz, 1H), 3.09-3.00 (m, 2H), 2.58 (m, 2H), 2.29 (d, J = 23.2 Hz, 6H). MS: 589.30 |
| 1-((2R)-4-((4-amino- 7-methyl-5-(4- phenoxyphenyl)-7H- pyrrolo[2,3- d]pyrimidin-6- yl)ethynyl)-4- hydroxy-2- methylpiperidin-1- yl)prop-2-en-1-one | 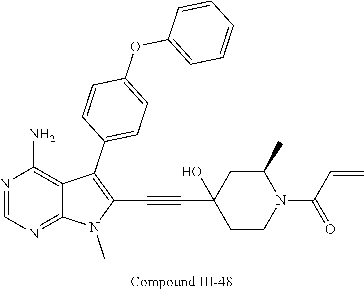 [SMILES: C=CC(=O)N1CCC(O)(C#Cc2c(-c3ccc(Oc4ccccc4)cc3)c3c(N)ncnc3n2C)C[C@H]1C] | ¹H NMR (400 MHz, DMSO-d₆) δ 8.19 (d, J = 2.1 Hz, 1H), 7.52 (d, J = 8.2 Hz, 2H), 7.44 (t, J = 7.9 Hz, 2H), 7.22-7.16 (m, 1H), 7.13 (d, J = 8.0 Hz, 4H), 6.76 (dd, J = 16.8, 10.5 Hz, 1H), 6.24 (br, 1H), 6.07 (d, J = 16.6 Hz, 1H), 5.77 (s, 1H), 5.64 (d, J = 10.6 Hz, 1H), 4.50 (s, 1H), 4.06 (s 1H), 3.72 (s, 3H), 3.20 (s, 1H), 1.93 (s, 3H), 1.78 (d, J= 13.3 Hz, 1H), 1.28 (d, J = 7.0 Hz, 3H). MS: 508.25 |
| (E)-1-(6-((4-amino- 5-(4- phenoxyphenyl)-7- (tetrahydrofuran-3- yl)-7H-pyrrolo[2,3- d]pyrimidin-6- yl)ethynyl)-2- azaspiro[3.3]heptan- 2-yl)-4- (dimethylamino)but- 2-en-1-one | 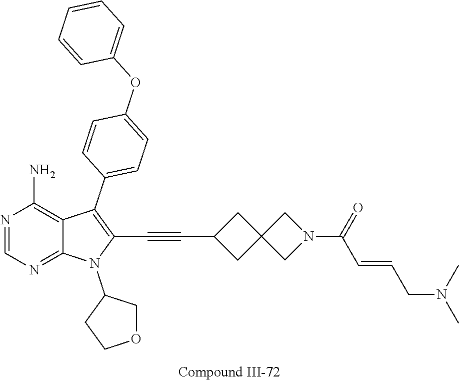 [SMILES: CN(C)C/C=C/C(=O)N1CC2(CC(C#Cc3c(-c4ccc(Oc5ccccc5)cc4)c4c(N)ncnc4n3C3CCOC3)C2)C1] | ¹H NMR (400 MHz, DMSO-d₆) δ 8.18 (s, 1H), 7.53-7.50 (dd, J = 8.4, 4.8 Hz, 2H), 7.45-7.41 (t, J = 7.4 Hz, 2H), 7.19-7.11 (m, 5H), 6.57- 6.52 (dt, J = 15.5, 6.0 Hz, 1H), 6.45- 6.1 (s, 1H) 6.06-5.98 (m, 1H), 5.50 (p, J = 8.2 Hz, 1H), 4.23-4.17 (d, J = 5.2 Hz, 2H), 4.11-4.06 (dd, J = 7.9, 2.7 Hz, 3H), 3.96-3.84 (m, 3H), 3.22-3.18 (p, J = 7.6 Hz, 1H), 3.01-2.97 (dd, J = 9.9, 6.2 Hz, 2H), 2.68-2.57 (m, 3H), 2.45-2.37 (dt, J = 11.7, 6.6 Hz, 1H), 2.36-2.26 (d, J = 10.9 Hz, 2H), 2.13-2.11 (d, J = 8.9 Hz, 6H). MS: 603.25 |
| (E)-1-(3-((4-amino- 7-methyl-5-(4- phenoxyphenyl)-7H- pyrrolo[2,3- d]pyrimidin-6- yl)ethynyl)azetidin- 1-yl)-4- (dimethylamino)but- 2-en-1-one | 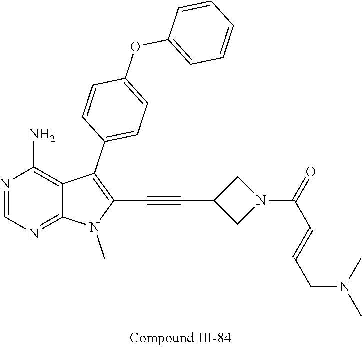 [SMILES: CN(C)C/C=C/C(=O)N1CC(C#Cc2c(-c3ccc(Oc4ccccc4)cc3)c3c(N)ncnc3n2C)C1] | ¹H NMR (400 MHz, DMSO-d₆) δ 8.20 (s, 1H), 7.54-7.52 (d, J = 8.1 Hz, 2H), 7.46-7.42 (t, J = 7.7 Hz, 2H), 7.20-7.17 (m, 1H), 7.14- 7.11 (m, 4H), 6.64-6.57 (m, 1H), 6.29 (br, 1H), 6.10-6.06 (d, J = 15.4 Hz, 1H), 4.55-4.51 (t, J = 8.6 Hz, 1H), 4.27-4.18 (dt, J = 19.4, 8.1 Hz, 2H), 3.91-3.87 (m, 1H), 3.84-3.76 (p, J = 7.6 Hz, 1H), 3.75 (s, 3H), M 3.33 (s, 1H), 3.01-3.00 (d, J = 6.1 Hz, 2H), 2.14 (s, 6H). MS: 507.25 |
| (E)-1-(6-((4-amino- 7-methyl-5-(4- phenoxyphenyl)-7H- pyrrolo[2,3- d]pyrimidin-6- yl)ethynyl)-2- azaspiro[3.3]heptan- 2-yl)-4- (dimethylamino)but- 2-en-1-one | 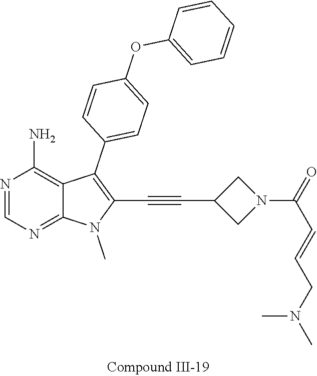 | ¹H NMR (400 MHz, DMSO-d₆) δ 8.18 (s, 1H), 7.55-7.47 (m, 2H), 7.42 (t, J = 7.4 Hz, 2H), 7.21-7.08 (m, 5H), 6.54 (dq, J = 15.3, 6.1 Hz, 1H), 6.22 (s, 1H), 6.08-5.95 (m, 1H), 4.19 (s, 1H), 4.09 (s, 1H), 3.89 (s, 1H), 3.85 (s, 1H), 3.71 (s, 3H), 3.21 (p, J = 7.8 Hz, 1H), 2.99 (td, J = 6.8, 3.3 Hz, 2H), 2.58 (d, J = 4.8 Hz, 2H), 2.30-2.22 (m, 2H), 2.12 (d, J = 9.0 Hz, 6H). MS: 547.25 |

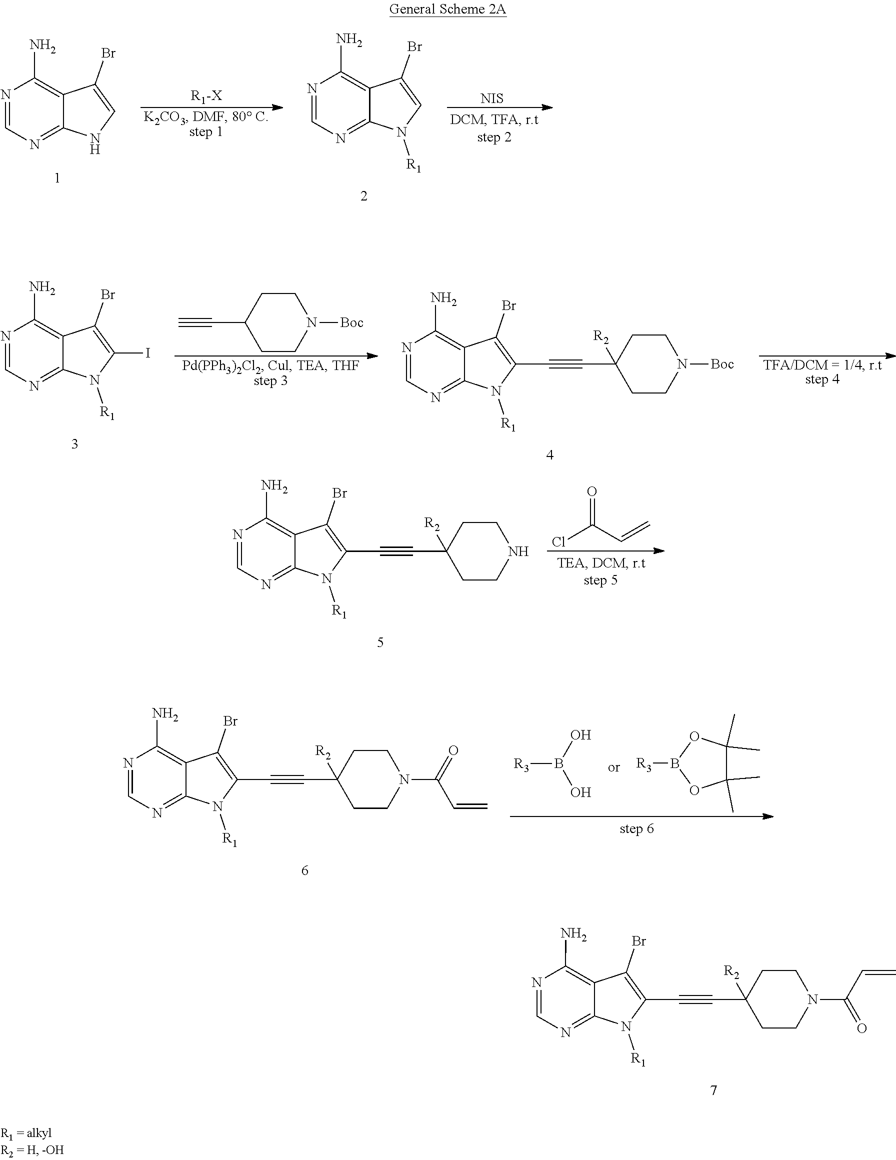

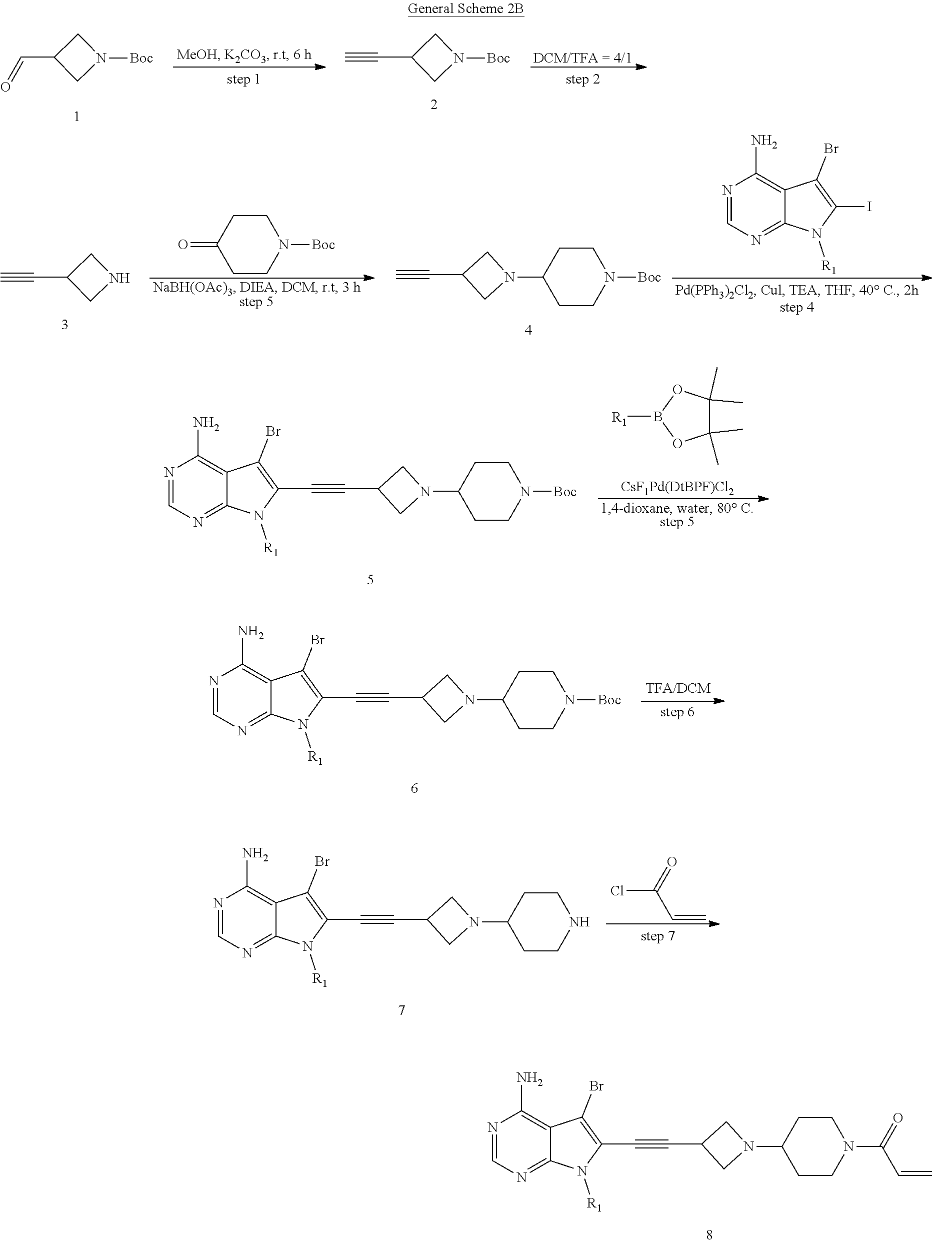

### Example 6

### 2-(4-(6-((1-acryloylpiperidin-4-yl)ethynyl)-4-amino-7-isopropyl-7H-pyrrolo[2,3-d]pyrimidin-5-yl)phenoxy)benzonitrile

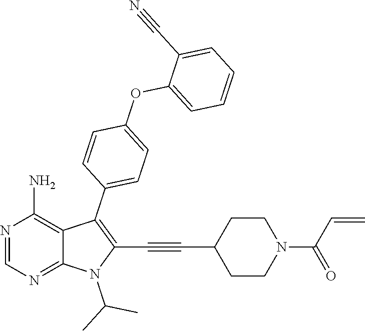 [SMILES: C=CC(=O)N1CCC(C#Cc2c(-c3ccc(Oc4ccccc4C#N)cc3)c3c(N)ncnc3n2C(C)C)CC1]

### 5-bromo-7-isopropyl-7H-pyrrolo[2,3-d]pyrimidin-4-amine

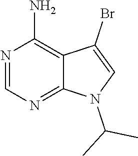

### Step 1

A round bottomed flask was charged with 5-bromo-7H-pyrrolo[2,3-d]pyrimidin-4-amine (6 g, 28.1 mmol), K₂CO₃ (11.6 g, 84.3 mmol), DMF (60 mL), 2-iodopropane (5.72 g, 33.7 mmol) and a stirbar. The solution was stirred for 1 h at 80° C. The reaction mixture was diluted with H₂O (200 mL), and the aqueous phase was extracted with ethyl acetate (200 mL) three times. The combined organic layers were washed with saturated brines, dried over sodium sulfate, filtered, and concentrated in vacuo. The resulting crude material was purified by silica gel chromatography (eluting with PE/EA=2/1). Concentration in vacuo resulted in 5-bromo-7-isopropyl-7H-pyrrolo[2,3-d]pyrimidin-4-amine (4.50 g, 63%) as a yellow amorphous solid.

### 5-bromo-6-iodo-7-isopropyl-7H-pyrrolo[2,3-d]pyrimidin-4-amine

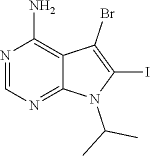

### Step 2

A round bottomed flask was charged with 5-bromo-7-isopropyl-7H-pyrrolo[2,3-d]pyrimidin-4-amine (4.5 g, 17.6 mmol), 1-iodopyrrolidine-2,5-dione (4.74 g, 21.1 mmol), DMF (50 mL) and a stirbar. The solution was stirred for 1 h at 80° C. The reaction mixture was diluted with saturated Na₂SO₃ solution (150 mL), and the aqueous phase was extracted with EA (150 mL) three times. The combined organic layers were washed with saturated brines, dried over sodium sulfate, filtered, and concentrated in vacuo. The resulting crude material was purified by silica gel chromatography (eluting with PE/EA=3/1). Concentration in vacuo resulted in 5-bromo-6-iodo-7-isopropyl-7H-pyrrolo[2,3-d]pyrimidin-4-amine (3.00 g, 45%) as a yellow amorphous solid.

### tert-butyl 4-((4-amino-5-bromo-7-isopropyl-7H-pyrrolo[2,3-d]pyrimidin-6-yl)ethynyl)piperidine-1-carboxylate

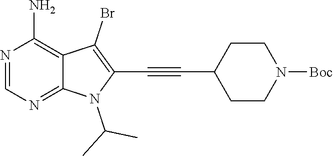 [SMILES: CC(C)n1c(C#CC2CCN(C(=O)OC(C)(C)C)CC2)c(Br)c2c(N)ncnc21]

### Step 3

A resealable reaction vial was charged with 5-bromo-6-iodo-7-isopropyl-7H-pyrrolo[2,3-d]pyrimidin-4-amine (3 g, 7.87 mmol), tert-butyl-4-ethynylpiperidine-1-carboxylate (3.28 g, 15.7 mmol), Pd(PPh₃)₂Cl₂ (1.65 g, 2.36 mmol), CuI (896 mg, 4.72 mmol), THE (50 mL), TEA (3.96 g, 39.3 mmol) and a stirbar before being evacuated and purged with nitrogen three times. The mixture was stirred for 2 h at room temperature. The reaction mixture was diluted with H₂O (150 mL), and the aqueous phase was extracted with EA (200 mL) three times. The combined organic layers were washed with saturated brines, dried over sodium sulfate, filtered, and concentrated in vacuo. The resulting crude material was purified by silica gel chromatography (eluting with PE/EA=2/1). Concentration in vacuo resulted in tert-butyl-4-((4-amino-5-bromo-7-isopropyl-7H-pyrrolo[2,3-d]pyrimidin-6-yl)ethynyl)piperidine-1-carboxylate (2.40 g, 66%) as a yellow amorphous solid.

### 5-bromo-7-isopropyl-6-(piperidin-4-ylethynyl)-7H-pyrrolo[2,3-d]pyrimidin-4-amine

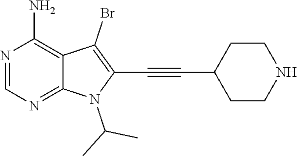 [SMILES: CC(C)n1c(C#CC2CCNCC2)c(Br)c2c(N)ncnc21]

### Step 4

A round bottomed flask was charged with tert-butyl-4-((4-amino-5-bromo-7-isopropyl-7H-pyrrolo[2,3-d]pyrimidin-6-yl)ethynyl)piperidine-1-carboxylate (2.4 g, 5.19 mmol), DCM (20 mL), TFA (5 mL) and a stirbar. The solution was stirred for 1 h at room temperature. The reaction mixture was diluted with H₂O (50 mL), and the pH of the mixture was adjusted to ˜9-10 with Na₂CO₃ aqueous phase was extracted with DCM (300 mL) three times. The combined organic layers were washed with saturated brines, dried over sodium sulfate, filtered, and concentrated in vacuo resulted in 5-bromo-7-isopropyl-6-(piperidin-4-ylethynyl)-7H-pyrrolo[2,3-d]pyrimidin-4-amine (1.80 g, 96%) as a yellow oil.

### 1-(4-((4-amino-5-bromo-7-isopropyl-7H-pyrrolo[2,3-d]pyrimidin-6-yl)ethynyl)piperidin-1-yl)prop-2-en-1-one

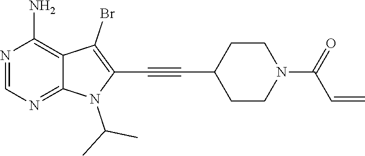 [SMILES: C=CC(=O)N1CCC(C#Cc2c(Br)c3c(N)ncnc3n2C(C)C)CC1]

### Step 5

A resealable reaction vial was charged with 5-bromo-7-isopropyl-6-(piperidin-4-ylethynyl)-7H-pyrrolo[2,3-d]pyrimidin-4-amine (1.8 g, 4.96 mmol), DCM (20 mL), TEA (1.49 g, 14.8 mmol) and a stirbar before being evacuated and purged with nitrogen three times. The mixture was cooled to 0° C. and acryloyl chloride (448 mg, 4.96 mmol) was added. The mixture was stirred for 1 h at 0° C. The reaction mixture was quenched with MeOH (5 mL) and diluted with H₂O (50 mL), and the aqueous phase was extracted with ethyl acetate (150 mL) three times. The combined organic layers were washed with saturated brines, dried over sodium sulfate, filtered, and concentrated in vacuo. The resulting crude material was purified by silica gel chromatography (eluting with PE/EA; 1/1). Concentration in vacuo resulted in 1-(4-((4-amino-5-bromo-7-isopropyl-7H-pyrrolo[2,3-d]pyrimidin-6-yl)ethynyl)piperidin-1-yl)prop-2-en-1-one (1.10 g, 53%) as a yellow amorphous solid.

### 2-(4-(6-((1-acryloylpiperidin-4-yl)ethynyl)-4-amino-7-isopropyl-7H-pyrrolo[2,3-d]pyrimidin-5-yl)phenoxy)benzonitrile

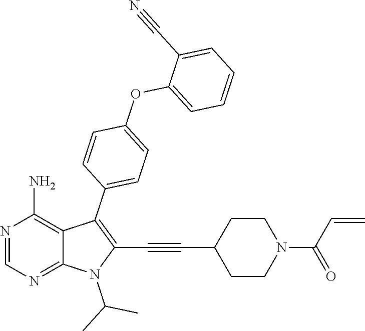 [SMILES: C=CC(=O)N1CCC(C#Cc2c(-c3ccc(Oc4ccccc4C#N)cc3)c3c(N)ncnc3n2C(C)C)CC1]

### Step 6

A resealable reaction vial was charged with 1-(4-((4-amino-5-bromo-7-isopropyl-7H-pyrrolo[2,3-d]pyrimidin-6-yl)ethynyl)piperidin-1-yl)prop-2-en-1-one (60 mg, 144 μmol), 2-(4-(4,4,5,5-tetramethyl-1,3,2-dioxaborolan-2-yl)phenoxy)benzonitrile (50.7 mg, 158 μmol), Pd(DtBPF)Cl₂ (9.37 mg, 14.4 μmol), K₂CO₃ (59.6 mg, 432 μmol), dioxane (4 mL), H₂O (1 mL) and a stirbar before being evacuated and purged with nitrogen three times. The mixture was stirred for 1 h at 80° C. The reaction mixture was diluted with H₂O (10 mL), and the aqueous phase was extracted with EA (20 mL) three times. The combined organic layers were washed with saturated brines, dried over sodium sulfate, filtered, and concentrated in vacuo. The resulting crude material was purified by silica gel chromatography (eluting with PE/EA=2/3). Concentration in vacuo. The resulting crude material was purified by HPLC. Column: XBridge Prep C18 OBD Column 19×150 mm 5 um; Mobile Phase A:Water (10 MMOL/L NH₄HCO₃), Mobile Phase B: ACN; Flow rate: 20 mL/min; Gradient: 50% B to 75% B in 7 min; 254&220 nm; Rt: 5.8 min. Lyophilization yielded 2-(4-(6-((1-acryloylpiperidin-4-yl)ethynyl)-4-amino-7-isopropyl-7H-pyrrolo[2,3-d]pyrimidin-5-yl)phenoxy)benzonitrile (10.3 mg, 19.4 μmol) as a yellow amorphous solid.

### Example 7

### 1-(4-(3-((4-amino-7-methyl-5-(3-methyl-4-phenoxyphenyl)-7H-pyrrolo[2,3-d]pyrimidin-6-yl)ethynyl)azetidin-1-yl)piperidin-1-yl)prop-2-en-1-one

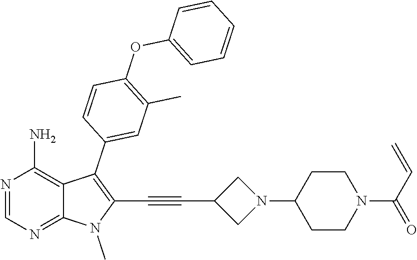 [SMILES: C=CC(=O)N1CCC(N2CC(C#Cc3c(-c4ccc(Oc5ccccc5)c(C)c4)c4c(N)ncnc4n3C)C2)CC1]

### tert-butyl 3-ethynylazetidine-1-carboxylate

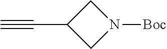

### Step 1

A round bottomed flask was charged with tert-butyl 3-formylazetidine-1-carboxylate (6.5 g, 35.0 mmol), K₂CO₃ (14.4 g, 105 mmol), MeOH (100 mL) and a stirbar. Then the solution was cooled to 0° C. and dimethyl (1-diazo-2-oxopropyl)phosphonate (10.0 g, 52.5 mmol) was added, and the solution was stirred for 16 h at room temperature. The reaction mixture was diluted with H₂O (100 mL), and the aqueous phase was extracted with DCM (200 mL) three times. The combined organic layers were washed with brines, dried over sodium sulfate, filtered, and concentrated in vacuo. The resulting crude material was purified by silica gel chromatography (eluting with EA/PE=1/10). Concentration in vacuo resulted in tert-butyl 3-ethynylazetidine-1-carboxylate (4.00 g, 63%) as a yellow oil.

### 3-ethynylazetidine

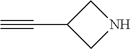

### Step 2

A round bottomed flask was charged with tert-butyl 3-ethynylazetidine-1-carboxylate (4 g, 22.0 mmol), DCM (16 mL), TFA (4 mL) and a stirbar. The solution was stirred for 4 h at room temperature. The solution was concentration in vacuo resulted in 3-ethynylazetidine (1.80 g, 22.1 mmol) as a yellow oil, without future purification and direct used next step.

### tert-butyl 4-(3-ethynylazetidin-1-yl)piperidine-1-carboxylate

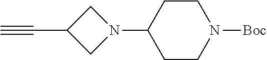 [SMILES: C#CC1CN(C2CCN(C(=O)OC(C)(C)C)CC2)C1]

### Step 3

A round bottomed flask was charged with 3-ethynylazetidine (1.8 g, 22.1 mmol), tert-butyl 4-oxopiperidine-1-carboxylate (8.80 g, 44.2 mmol), DCM (100 mL), DIEA (14.1 g, 110 mmol) and a stirbar. The mixture was cooled to 0° C. and NaBH(OAc)₃ (9.37 g, 44.2 mmol) was added, the solution was stirred for 3 h at room temperature. The reaction mixture was diluted with H₂O (100 mL), and the aqueous phase was extracted with DCM (200 mL) three times. The combined organic layers were washed with brines, dried over sodium sulfate, filtered, and concentrated in vacuo. The resulting crude material was purified by silica gel chromatography (eluting with EA/PE=2/1). Concentration in vacuo resulted in tert-butyl 4-(3-ethynylazetidin-1-yl)piperidine-1-carboxylate (5.40 g, 92.4%) as a yellow oil.

### tert-butyl-4-(3-((4-amino-5-bromo-7-methyl-7H-pyrrolo[2,3-d]pyrimidin-6-yl)ethynyl)azetidin-1-yl)piperidine-1-carboxylate

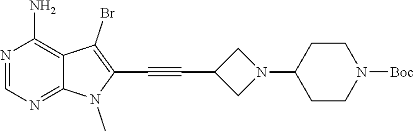 [SMILES: Cn1c(C#CC2CN(C3CCN(C(=O)OC(C)(C)C)CC3)C2)c(Br)c2c(N)ncnc21]

### Step 4

A resealable reaction vial was charged with 5-bromo-6-iodo-7-methyl-7H-pyrrolo[2,3-d]pyrimidin-4-amine (1.5 g, 4.24 mmol), tert-butyl 4-(3-ethynylazetidin-1-yl)piperidine-1-carboxylate (1.68 g, 6.36 mmol), Pd(PPh₃)₂Cl₂ (593 mg, 848 μmol), CuI (321 mg, 1.69 mmol), THE (20 mL), TEA (4.28 g, 42.4 mmol) and a stirbar before being evacuated and purged with nitrogen three times. The mixture was stirred for 2 h at 40° C. The reaction mixture was diluted with H₂O (100 mL), and the aqueous phase was extracted with EA (150 mL) three times. The combined organic layers were washed with brines, dried over sodium sulfate, filtered, and concentrated in vacuo. The resulting crude material was purified by silica gel chromatography (eluting with MeOH/DCM=1/40). Concentration in vacuo resulted in tert-butyl 4-[3-(2-{4-amino-5-bromo-7-methyl-7H-pyrrolo[2,3-d]pyrimidin-6-yl}ethynyl)azetidin-1-yl]piperidine-1-carboxylate (1.50 g, 72.4%) as a yellow amorphous solid.

### tert-butyl-4-(3-((4-amino-7-methyl-5-(3-methyl-4-phenoxyphenyl)-7H-pyrrolo[2,3-d]pyrimidin-6-yl)ethynyl)azetidin-1-yl)piperidine-1-carboxylate

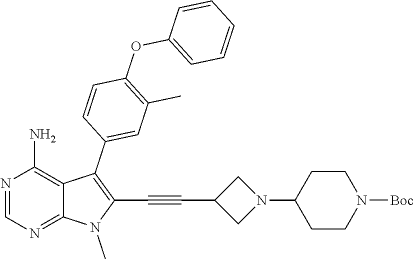 [SMILES: Cc1cc(-c2c(C#CC3CN(C4CCN(C(=O)OC(C)(C)C)CC4)C3)n(C)c3ncnc(N)c23)ccc1Oc1ccccc1]

### Step 5

To a stirred solution of tert-butyl 4-[3-(2-[4-amino-5-bromo-7-methyl-7H-pyrrolo[2,3-d]pyrimidin-6-yl]ethynyl)azetidin-1-yl]piperidine-1-carboxylate (200 mg, 0.41 mmol) and 4,4,5,5-tetramethyl-2-(3-methyl-4-phenoxyphenyl)-1,3,2-dioxaborolane (253.5 mg, 0.82 mmol) in dioxane (4 mL) were added Pd(DtBPF)Cl₂(26.6 mg, 0.04 mmol), H₂O (1 mL) and CsF (186.2 mg, 1.23 mmol). The resulting mixture was stirred for 3 h at 80° C. under nitrogen atmosphere. The resulting mixture was extracted with EtOAc. The combined organic layers were dried over anhydrous Na₂SO₄. After filtration, the filtrate was concentrated under reduced pressure. The residue was purified by Prep-TLC (PE/EtOAc 1:1) to afford tert-butyl 4-(3-[2-[4-amino-7-methyl-5-(3-methyl-4-phenoxyphenyl)-7H-pyrrolo[2,3-d]pyrimidin-6-yl]ethynyl]azetidin-1-yl)piperidine-1-carboxylate (100 mg, 41.3%) as a brown solid.

### 7-methyl-5-(3-methyl-4-phenoxyphenyl)-6-((1-(piperidin-4-yl)azetidin-3-yl)ethynyl)-7H-pyrrolo[2,3-d]pyrimidin-4-amine

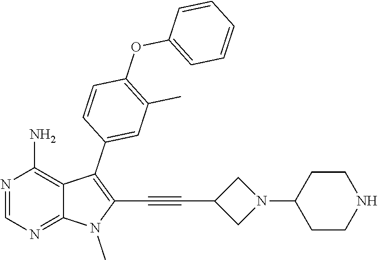 [SMILES: Cc1cc(-c2c(C#CC3CN(C4CCNCC4)C3)n(C)c3ncnc(N)c23)ccc1Oc1ccccc1]

### Step 6

To a stirred solution of tert-butyl 4-(3-[2-[4-amino-7-methyl-5-(3-methyl-4-phenoxyphenyl)-7H-pyrrolo[2,3-d]pyrimidin-6-yl]ethynyl]azetidin-1-yl)piperidine-1-carboxylate (100 mg, 1 equiv) in DCM (0.8 mL) was added TFA (0.2 mL). The resulting mixture was stirred for 1 h at room temperature. The mixture was basified to pH 7 with saturated NaHCO₃ (aq.). The resulting mixture was extracted with CH₂Cl₂. The combined organic layers were dried over anhydrous Na₂SO₄. After filtration, the filtrate was concentrated under reduced pressure to afford 7-methyl-5-(3-methyl-4-phenoxyphenyl)-6-[2-[1-(piperidin-4-yl)azetidin-3-yl]ethynyl]-7H-pyrrolo[2,3-d]pyrimidin-4-amine (50 mg, 60.2%) as a brown solid.

### 1-(4-(3-((4-amino-7-methyl-5-(3-methyl-4-phenoxyphenyl)-7H-pyrrolo[2,3-d]pyrimidin-6-yl)ethynyl)azetidin-1-yl)piperidin-1-yl)prop-2-en-1-one

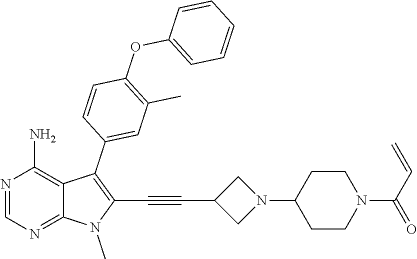 [SMILES: C=CC(=O)N1CCC(N2CC(C#Cc3c(-c4ccc(Oc5ccccc5)c(C)c4)c4c(N)ncnc4n3C)C2)CC1]

### Step 7

To a stirred solution of 7-methyl-5-(3-methyl-4-phenoxyphenyl)-6-[2-[1-(piperidin-4-yl)azetidin-3-yl]ethynyl]-7H-pyrrolo[2,3-d]pyrimidin-4-amine (50 mg, 0.10 mmol) and TEA (30.8 mg, 0.30 mmol) in DCM (2 mL) were added prop-2-enoyl chloride (8.3 mg, 0.09 mmol) dropwise at 0° C. The resulting mixture was stirred for 3 h at 0° C. The resulting mixture was concentrated under vacuum. The crude product was purified by Prep-HPLC with the following conditions (Column: XBridge Shield RP18 OBD Column 30*150 mm, 5 um; Mobile Phase A:Water (10 MMOL/L NH₄HCO₃), Mobile Phase B: ACN; Flow rate: 60 mL/min; Gradient: 25% B to 55% B in 7 min; 254 nm; Rt: 6.40 min) to afford 1-[4-(3-[2-[4-amino-7-methyl-5-(3-methyl-4-phenoxyphenyl)-7H-pyrrolo[2,3-d]pyrimidin-6-yl]ethynyl]azetidin-1-yl)piperidin-1-yl]prop-2-en-1-one (14.8 mg, 26.7%) as a white solid.

Compounds prepared by substantially the same methods, and their characterization data, are listed in Table 6, below.

TABLE 6
Selected Compounds

| Compound | Structure | Proton NMR & Mass Spec. (obs.) |
|---|---|---|
| 1-(4-((4-amino-5-(4- (3-chlorophenoxy)- phenyl)-7-isopropyl- 7H-pyrrolo[2,3-d] pyrimidin-6-yl)eth- ynyl)piperidin-1-yl)- prop-2-en-1-one | 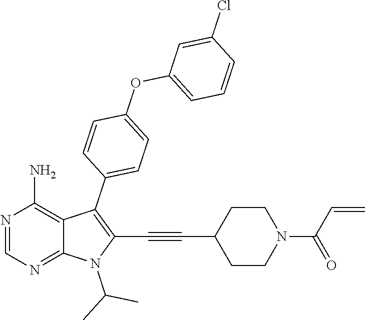 [SMILES: C=CC(=O)N1CCC(C#Cc2c(-c3ccc(Oc4cccc(Cl)c4)cc3)c3c(N)ncnc3n2C(C)C)CC1] | ¹H NMR (400 MHz, DMSO) δ 8.16 (s, 1H), 7.54-7.51 (m, 2H), 7.42 (t, J = 8.1 Hz, 1H), 7.22-7.14 (m, 4H), 7.07 (dd, J = 8.4, 2.4 Hz, 1H), 6.77 (dd, J = 16.7, 10.5 Hz, 1H), 6.08 (dd, J = 16.7, 2.4 Hz, 2H), 5.66 (dd, J = 10.4, 2.4 Hz, 1H), 3.69 (s, 2H), 3.49-3.38 (s, 2H), 3.02 (dt, J = 8.0, 4.0 Hz, 1H), 1.80 (s, 2H), 1.61 (d, J = 6.9 Hz, 6H), 1.52 (s, 2H). MS: 540.2 |
| 1-(4-((4-amino-5-(4- (3-fluorophenoxy)- phenyl)-7-isopropyl- 7H-pyrrolo[2,3-d] pyrimidin-6-yl)eth- ynyl)piperidin-1-yl)- prop-2-en-1-one | 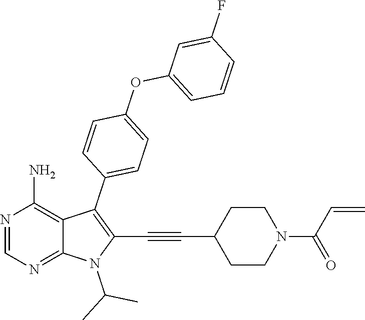 [SMILES: C=CC(=O)N1CCC(C#Cc2c(-c3ccc(Oc4cccc(F)c4)cc3)c3c(N)ncnc3n2C(C)C)CC1] | ¹H NMR (400 MHz, DMSO) δ 8.16 (s, 1H), 7.53-7.46 (m, 2H), 7.46- 7.40 (m, 1H), 7.16-7.14 (m, 2H), 7.01-6.91 (m, 3H), 6.77 (dd, J = 16.7, 10.5 Hz, 1H), 6.08 (dd, J = 16.7, 2.4 Hz, 1H), 5.65 (dd, J = 10.5, 2.4 Hz, 1H), 5.11 (p, J = 6.9 Hz, 1H), 3.68 (s, 2H), 3.41 (s, 2H), 3.02 (dt, J = 8.0, 4.0 Hz, 1H), 1.80 (s, 2H), 1.61 (d, J = 6.9 Hz, 6H), 1.52 (s, 2H). MS: 524.2 |
| 1-(4-((4-amino-7- isopropyl-5-(4-(m- tolyloxy)phenyl)- 7H-pyrrolo[2,3-d] pyrimidin-6-yl)eth- ynyl)piperidin-1-yl)- prop-2-en-1-one | 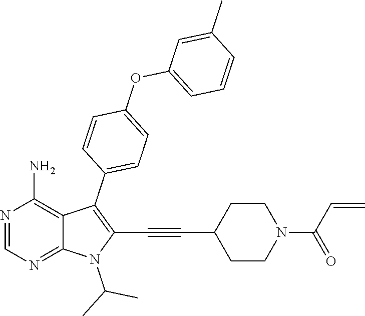 [SMILES: C=CC(=O)N1CCC(C#Cc2c(-c3ccc(Oc4cccc(C)c4)cc3)c3c(N)ncnc3n2C(C)C)CC1] | ¹H NMR (400 MHz, DMSO) δ 8.16 (s, 1H), 7.50-7.48 (m, 2H), 7.29 (t, J = 7.8 Hz, 1H), 7.09-7.07 (m, 2H), 6.99 (d, J = 7.7 Hz, 1H), 6.93 (d, J = 2.2 Hz, 1H), 6.87 (dd, J = 8.1, 2.5 Hz, 1H), 6.78 (dd, J = 16.7, 10.5 Hz, 1H), 6.08 (dd, J = 16.7, 2.4 Hz, 1H), 5.65 (dd, J = 10.5, 2.5 Hz, 1H), 5.11 (p, J = 6.9 Hz, 1H), 3.66 (s, 2H), 3.47-3.38 (s, 2H), 3.02 (dt, J = 7.9, 3.9 Hz, 1H), 2.31 (s, 3H), 1.79 (s, 2H), 1.61 (d, J = 6.9 Hz, 6H), 1.52 (s, 2H). MS: 520.2 |
| 1-(4-((4-amino-7- isopropyl-5-(4-(6- methylpyridin-2-yl- oxy)phenyl)-7H-pyr- rolo[2,3-d]pyrimi- din-6-yl)ethynyl)- piperidin-1-yl)prop- 2-en-1-one | 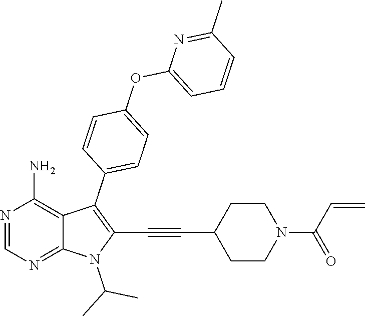 [SMILES: C=CC(=O)N1CCC(C#Cc2c(-c3ccc(Oc4cccc(C)n4)cc3)c3c(N)ncnc3n2C(C)C)CC1] | ¹H NMR (300 MHz, DMSO-d₆) δ 8.17 (s, 1H), 7.78-7.73 (m, 1H), 7.54-7.51 (m, 2H), 7.22-7.19 (m, 2H), 7.04 (d, J = 7.4 Hz, 1H), 6.83- 6.72 (m, 2H), 6.09 (dd, J = 16.7, 2.5 Hz, 2H), 5.64 (dd, J = 10.4, 2.4 Hz, 1H), 5.13 (p, J = 7.0 Hz, 1H), 3.74 (s, 2H), 3.42 (s, 2H), 3.04 (dt, J = 8.1, 4.0 Hz, 1H), 2.36 (s, 3H), 1.82 (s, 2H), 1.61 (d, J = 6.9 Hz, 6H), 1.53 (s, 2H). MS: 521.2 |
| 2-(4-(6-((1-acrylo- ylpiperidin-4-yl)- ethynyl)-4-amino- 7-isopropyl-7H-pyr- rolo[2,3-d]pyrimi- din-5-yl)phenoxy)- benzonitrile | 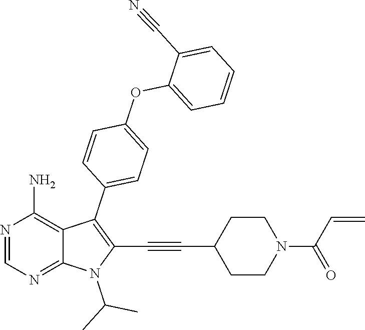 [SMILES: C=CC(=O)N1CCC(C#Cc2c(-c3ccc(Oc4ccccc4C#N)cc3)c3c(N)ncnc3n2C(C)C)CC1] | ¹H NMR (400 MHz, DMSO-d₆) δ 8.17 (s, 1H), 7.93 (dd, J = 7.8, 1.7 Hz, 1H), 7.70 (ddd, J = 8.9, 7.5, 1.7 Hz, 1H), 7.58-7.56 (m, 2H), 7.33- 7.24 (m, 3H), 7.14 (d, J = 8.5 Hz, 1H), 6.78 (dd, J = 16.7, 10.5 Hz, 1H), 6.08 (dd, J = 16.7, 2.4 Hz, 1H), 5.66 (dd, J = 10.5, 2.4 Hz, 1H), 5.12 (p, J = 6.9 Hz, 1H), 3.71 (s, 2H), 3.43 (s, 2H), 3.03 (dq, J = 8.1, 4.1 Hz, 1H), 1.82 (s, 2H), 1.63 (d, J = 6.9 Hz, 6H), 1.54 (s, 2H). MS: 531.3 |
| 1-(4-((4-amino-7- isopropyl-5-(4-(pyr- idin-2-yloxy)phenyl)- 7H-pyrrolo[2,3-d] pyrimidin-6-yl)eth- ynyl)piperidin-1-yl)- prop-2-en-1-one | 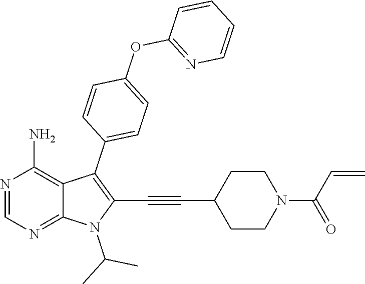 [SMILES: C=CC(=O)N1CCC(C#Cc2c(-c3ccc(Oc4ccccn4)cc3)c3c(N)ncnc3n2C(C)C)CC1] | ¹H NMR (400 MHz, DMSO) δ 8.20 (ddd, J = 4.9, 2.0, 0.8 Hz, 1H), 8.17 (s, 1H), 7.88 (ddd, J = 8.3, 7.2, 2.0 Hz, 1H), 7.55-7.52 (m, 2H), 7.25- 7.22 (m, 2H), 7.17 (ddd, J = 7.2, 4.9, 1.0 Hz, 1H), 7.08 (dt, J = 8.3, 0.9 Hz, 1H), 6.77 (dd, J = 16.7, 10.5 Hz, 1H), 6.07 (dd, J = 16.7, 2.4 Hz, 1H), 5.64 (dd, J = 10.4, 2.4 Hz, 1H), 5.12 (p, J = 6.8 Hz, 1H), 3.69 (s, 2H), 3.41 (s, 2H), 3.02 (dq, J = 8.0, 4.0 Hz, 1H), 1.81 (s, 2H), 1.62 (d, J = 6.9 Hz, 6H), 1.53 (s, 2H). MS: 507.2 |
| 1-(4-((4-amino-7- isopropyl-5-(4-(pyr- idin-3-yloxy)phenyl)- 7H-pyrrolo[2,3-d] pyrimidin-6-yl)eth- ynyl)piperidin-1-yl)- prop-2-en-1-one | 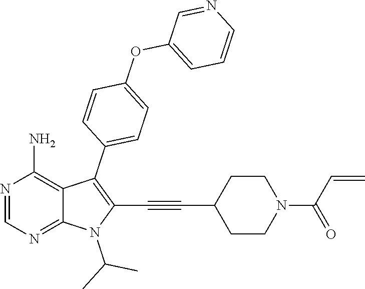 [SMILES: C=CC(=O)N1CCC(C#Cc2c(-c3ccc(Oc4cccnc4)cc3)c3c(N)ncnc3n2C(C)C)CC1] | ¹H NMR (400 MHz, DMSO) δ 8.47 (d, J = 2.8 Hz, 1H), 8.40 (d, J = 4.7 Hz, 1H), 8.16 (s, 1H), 7.58-7.50 (m, 3H), 7.46 (dd, J = 8.4, 4.6 Hz, 1H), 7.16 (d, J = 8.2 Hz, 2H), 6.79 (dd, J = 16.7, 10.5 Hz, 1H), 6.30 (s, 2H), 6.09 (dd, J = 16.6, 2.4 Hz, 1H), 5.66 (dd, J = 10.5, 2.4 Hz, 1H), 5.12 (p, J = 7.1 Hz, 1H), 3.69 (s, 2H), 3.42 (s, 2H), 3.03 (s, 1H), 1.81 (s, 2H), 1.62 (d, J = 6.9 Hz, 6H), 1.53 (s, 2H). MS: 507.25 |
| 1-(4-((4-amino-7- isopropyl-5-(5-phen- oxypyridin-2-yl)-7H- pyrrolo[2,3-d]pyr- imidin-6-yl)ethyn- yl)piperidin-1-yl)- prop-2-en-1-one | 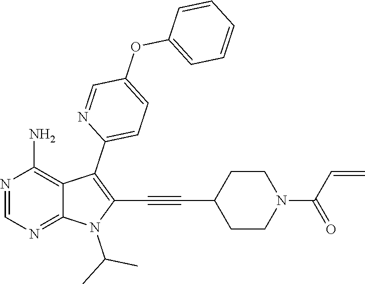 [SMILES: C=CC(=O)N1CCC(C#Cc2c(-c3ccc(Oc4ccccc4)cn3)c3c(N)ncnc3n2C(C)C)CC1] | ¹H NMR (400 MHz, Methanol-d₄) δ 8.44-8.34 (m, 2H), 8.11 (s, 1H), 7.54-7.42 (m, 3H), 7.24 (t, J = 7.4 Hz, 1H), 7.14 (d, J = 8.0 Hz, 2H), 6.79 (dd, J = 16.7, 10.7 Hz, 1H), 6.21 (d, J = 16.9 Hz, 1H), 5.80-5.72 (m, 1H), 5.32-5.22 (m, 1H), 4.09 (s, 1H), 3.95 (d, J = 14.3 Hz, 1H), 3.52 (d, J = 13.0 Hz, 1H), 3.40 (m, 1H), 3.23-3.15 (m, 1H), 2.06 (s, 2H), 1.78 (s, 2H), 1.72 (d, J = 7.0 Hz, 6H). MS: 507.15 |
| 1-(4-((4-amino-7- isopropyl-5-(6-phen- oxypyridin-3-yl)-7H- pyrrolo[2,3-d]pyrim- idin-6-yl)ethynyl)- piperidin-1-yl)prop- 2-en-1-one | 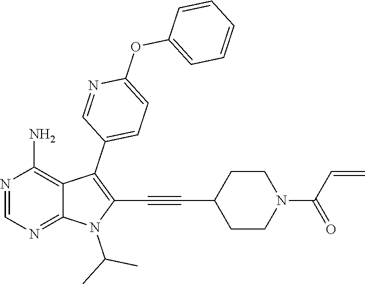 [SMILES: C=CC(=O)N1CCC(C#Cc2c(-c3ccc(Oc4ccccc4)nc3)c3c(N)ncnc3n2C(C)C)CC1] | ¹H NMR (400 MHz, DMSO-d₆) δ 8.26-8.20 (m, 1H), 8.17 (s, 1H), 7.92 (dd, J = 8.5, 2.5 Hz, 1H), 7.49- 7.39 (m, 2H), 7.27-7.15 (m, 3H), 7.11 (dd, J = 8.5, 0.7 Hz, 1H), 6.81 (dd, J = 16.7, 10.5 Hz, 1H), 6.29 (s, 2H), 6.11 (dd, J = 16.6, 2.4 Hz, 1H), 5.68 (dd, J = 10.4, 2.4 Hz, 1H), 5.11 (p, J = 6.9 Hz, 1H), 3.70 (s, 2H), 3.43 (d, J = 10.1 Hz, 1H), 3.2 (m, 1H) 3.03 (tt, J = 8.0, 4.1 Hz, 1H), 1.81 (s, 2H), 1.62 (d, J = 6.9 Hz, 6H), 1.51 (d, J = 10.7 Hz, 2H). MS: 507.25 |
| 1-(4-((4-amino-7- isopropyl-5-(4-(pyr- imidin-2-yloxy)- phenyl)-7H-pyrrolo [2,3-d]pyrimidin-6- yl)ethynyl)piperidin- 1-yl)prop-2-en-1-one | 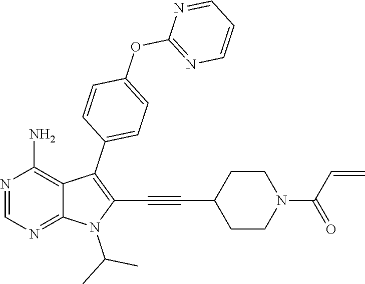 [SMILES: C=CC(=O)N1CCC(C#Cc2c(-c3ccc(Oc4ncccn4)cc3)c3c(N)ncnc3n2C(C)C)CC1] | ¹H NMR (300 MHz, DMSO-d₆) δ 8.69 (d, J = 4.8 Hz, 2H), 8.18 (s, 1H), 7.58-7.55 (m, 2H), 7.30 (dd, J = 9.2, 4.8 Hz, 3H), 6.77 (dd, J = 16.7, 10.5 Hz, 1H), 6.07 (dd, J = 16.7, 2.5 Hz, 2H), 5.64 (dd, J = 10.5, 2.5 Hz, 1H), 5.14 (p, J = 6.8 Hz, 1H), 3.71 (s, 2H), 3.43 (s, 2H), 3.04 (dt, J = 8.1, 4.1 Hz, 1H), 1.81 (s, 2H), 1.63 (d, J = 6.9 Hz, 6H), 1.55 (s, 2H). MS: 508.2 |
| 1-(4-((4-amino-7- isopropyl-5-(4-(pyr- azin-2-yloxy)phenyl)- 7H-pyrrolo[2,3-d] pyrimidin-6-yl)eth- ynyl)piperidin-1- yl)prop-2-en-1-one | 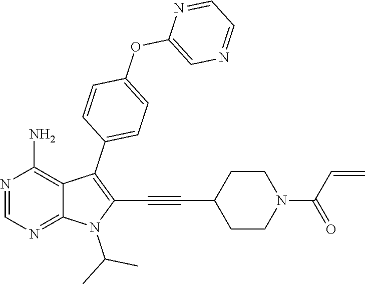 [SMILES: C=CC(=O)N1CCC(C#Cc2c(-c3ccc(Oc4cnccn4)cc3)c3c(N)ncnc3n2C(C)C)CC1] | ¹H NMR (400 MHz, DMSO) δ 8.60 (d, J =1.4 Hz, 1H), 8.42 (d, J = 2.7 Hz, 1H), 8.27 (dd, J = 2.7, 1.4 Hz, 1H), 8.18 (s, 1H), 7.60-7.53 (m, 2H), 7.37-7.29 (m, 2H), 6.78 (dd, J = 16.7, 10.5 Hz, 1H), 6.07 (dd, J = 16.7, 2.4 Hz, 1H), 6.02 (s, 2H), 5.64 (dd, J = 10.5, 2.4 Hz, 1H), 5.13 (p, J = 6.9 Hz, 1H), 3.69 (d, J = 19.4 Hz, 2H), 3.43 (d, J = 10.3 Hz, 1H), 3.03 (dq, J = 7.9, 4.0 Hz, 1H), 1.82 (s, 2H), 1.63 (d, J = 6.9 Hz, 6H), 1.54 (s, 2H). MS: 508.20 |
| 1-(4-((4-amino-7- isopropyl-5-(5-phen- oxypyrazin-2-yl)- 7H-pyrrolo[2,3-d] pyrimidin-6-yl)eth- ynyl)piperidin-1- yl)prop-2-en-1-one | 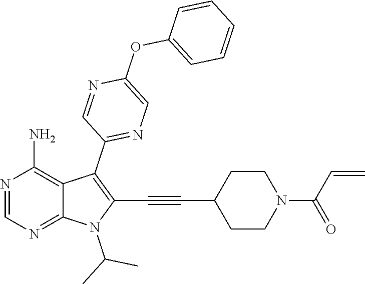 [SMILES: C=CC(=O)N1CCC(C#Cc2c(-c3cnc(Oc4ccccc4)cn3)c3c(N)ncnc3n2C(C)C)CC1] | ¹H NMR (400 MHz, Methanol-d₄) δ 9.11 (d, J = 1.4 Hz, 1H), 8.50 (d, J = 1.3 Hz, 1H), 8.17 (s, 1H), 7.54-7.43 (m, 2H), 7.30 (t, J = 7.5 Hz, 1H), 7.27-7.19 (m, 2H), 6.77 (dd, J = 16.8, 10.7 Hz, 1H), 6.21 (dd, J = 16.8, 2.0 Hz, 1H), 5.76 (dd, J = 10.7, 2.0 Hz, 1H), 5.26 (p, J = 7.0 Hz, 1H), 4.07 (d, J = 13.6 Hz, 1H), 3.92 (d, J = 14.6 Hz, 1H), 3.49 (t, J = 11.2 Hz, 1H), 3.40 (d, J = 10.8 Hz, 1H), 3.18 (dt, J = 8.7, 4.5 Hz, 1H), 1.99 (d, J = 29.1 Hz, 2H), 1.79-1.70 (m, 8H). MS: 508.2 |
| 1-(4-((4-amino-7- isopropyl-5-(2-phen- oxypyrimidin-5-yl)- 7H-pyrrolo[2,3-d] pyrimidin-6-yl)eth- ynyl)piperidin-1- yl)prop-2-en-1-one | 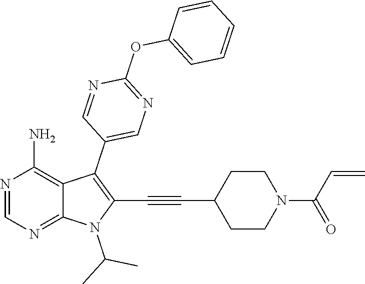 [SMILES: C=CC(=O)N1CCC(C#Cc2c(-c3cnc(Oc4ccccc4)nc3)c3c(N)ncnc3n2C(C)C)CC1] | ¹H NMR (300 MHz, DMSO) δ 8.65 (s, 2H), 8.17 (s, 1H), 7.45 (t, J = 7.8 Hz, 2H), 7.25 (dd, J = 10.0, 8.6 Hz, 3H), 6.81 (dd, J = 16.7, 10.5 Hz, 1H), 6.53 (s, 2H), 6.10 (dd, J = 16.7, 2.5 Hz, 1H), 5.68 (dd, J = 10.5, 2.4 Hz, 1H), 5.13-5.09 (m, 1H), 3.70 (s, 2H), 3.43 (s, 1H), 3.26 (s, 1H), 3.03 (s, 1H), 1.81 (s, 2H), 1.61 (d, J = 6.9 Hz, 6H), 1.51 (s, 2H). MS: 508.2 |
| 1-(3-(4-((4-amino-7- isopropyl-5-(4-(pyr- idin-2-yloxy)phenyl)- 7H-pyrrolo[2,3-d] pyrimidin-6-yl)eth- ynyl)piperidin-1-yl)- azetidin-1-yl)prop-2- en-1-one formate | 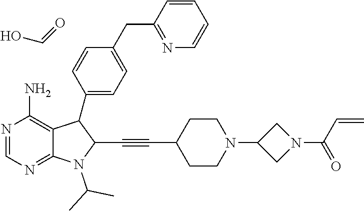 | ¹H NMR (400 MHz, DMSO) δ 8.21 (dd, J = 5.0, 2.0 Hz, 2H), 8.17 (s, 1H), 7.91-7.86 (m, 1H), 7.54-7.52 (m, 2H), 7.26-7.23 (m, 2H), 7.16 (dd, J = 7.1, 4.9 Hz, 1H), 7.09 (d, J = 8.3 Hz, 1H), 6.28 (dd, J = 17.0, 10.3 Hz, 1H), 6.08 (dd, J = 17.0, 2.3 Hz, 1H), 5.65 (dd, J = 10.2, 2.3 Hz, 1H), 5.13 (p, J = 6.9 Hz, 1H), 4.18 (t, J = 8.0 Hz, 1H), 3.96 (dd, J = 8.9, 5.0 Hz, 1H), 3.88 (dd, J = 10.2, 7.2 Hz, 1H), 3.68 (dd, J = 10.4, 5.1 Hz, 1H), 3.10-3.05 (m, 1H), 2.77 (s, 1H), 2.42 |
| | | (s, 2H), 2.12 (s, 2H), 1.82 (s, 2H), |
| | | 1.62 (d, J = 6.9 Hz, 8H). MS: 562.3 |
| 1-(3-(4-((4-amino-7- isopropyl-5-(4-(pyr- idin-2-ylamino)phen- yl)-7H-pyrrolo[2,3- d]pyrimidin-6-yl)- ethynyl)piperidin-1- yl)azetidin-1-yl)prop- 2-en-1-one | 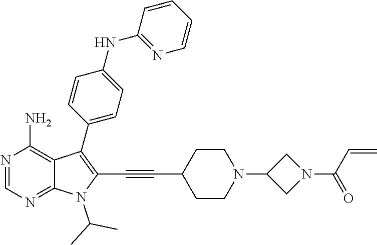 [SMILES: C=CC(=O)N1CC(N2CCC(C#Cc3c(-c4ccc(Nc5ccccn5)cc4)c4c(N)ncnc4n3C(C)C)CC2)C1] | ¹H NMR (400 MHz, DMSO) δ 9.21 (s, 1H), 8.23-8.13 (m, 2H), 7.89- 7.82 (m, 2H), 7.59 (ddd, J = 8.8, 7.2, 2.0 Hz, 1H), 7.44-7.36 (m, 2H), 6.88 (d, J = 8.4 Hz, 1H), 6.81-6.73 (m, 1H), 6.26 (dd, J = 17.0, 10.2 Hz, 1H), 6.08 (dd, J = 17.0, 2.3 Hz, 1H), 5.65 (dd, J = 10.2, 2.3 Hz, 1H), 5.11 (p, J = 6.9 Hz, 1H), 4.14 (t, J = 8.0 Hz, 1H), 3.94 (dd, J = 8.9, 5.1 Hz, 1H), 3.88 (dd, J = 10.2, 7.2 Hz, 1H), 3.66 (dd, J = 10.3, 5.2 Hz, 1H), 3.06 (h, J = 6.6, 5.8 Hz, 1H), 2.79 (s, 1H), 2.42 (s, 2H), 2.14 (s, 2H), 1.82 (s, 2H), 1.63 (d, J = 6.9 Hz, 6H), 1.60- |
| | | 1.53 (m, 2H). MS: 561.15 |
| 1-(4-((4-amino-5-(4- (cyclopentylmeth- oxy)phenyl)-7-iso- propyl-7H-pyrrolo [2,3-d]pyrimidin-6- yl)ethynyl)piperidin- 1-yl)prop-2-en-1-one | 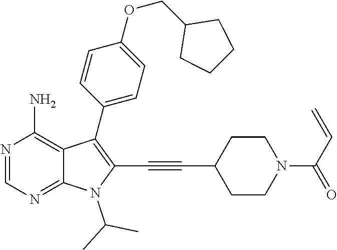 [SMILES: C=CC(=O)N1CCC(C#Cc2c(-c3ccc(OCC4CCCC4)cc3)c3c(N)ncnc3n2C(C)C)CC1] | ¹H NMR (400 MHz, DMSO-d₆) δ 8.142 (s, 1H), 7.409-7.387 (m, 2H), 7.037-7.015 (m, 2H), 6.819-6.751 (dd, J = 16.7, 10.5 Hz, 1H), 6.108- 6.060 (dd, J = 16.7, 2.5 Hz, 1H), 5.675-5.643 (dd, J = 10.5, 2.4 Hz, 1H), 5.134-5.065 (p, J = 6.8 Hz, 1H), 3.882-3.865 (d, J = 7.0 Hz, 2H), 3.663 (s, 2H), 3.334 (s, 2H), 3.032-2.972 (dt, J = 8.0, 3.9 Hz, 1H), 2.357-2.283 (p, J = 7.4 Hz, 1H), 1.797-1.778 (d, J = 8.1 Hz, 4H), 1.616-1.599 (d, J = 6.9 Hz, 12H), 1.567-1.529 (dq, J = 13.2, 6.9, 6.1 Hz, 2H). MS: 512.30 |
| 1-(4-((4-amino-7- isopropyl-5-(4-(phen- ylamino)phenyl)-7H- pyrrolo[2,3-d]pyrim- idin-6-yl)ethynyl)- piperidin-1-yl)prop- 2-en-1-one | 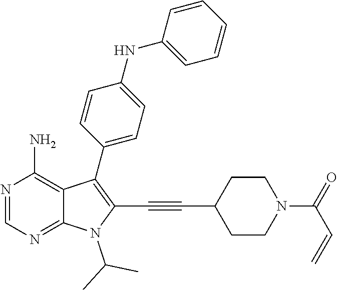 [SMILES: C=CC(=O)N1CCC(C#Cc2c(-c3ccc(Nc4ccccc4)cc3)c3c(N)ncnc3n2C(C)C)CC1] | ¹H NMR (400 MHz, DMSO) δ 8.35 (s, 1H), 8.14 (s, 1H), 7.36 (d, J = 8.5 Hz, 2H), 7.26 (dd, J = 8.4, 7.2 Hz, 2H), 7.17-7.11 (m, 4H), 6.88-6.75 (m, 2H), 6.07 (dd, J = 16.7, 2.4 Hz, 1H), 5.64 (dd, J = 10.5, 2.4 Hz, 1H), 5.10 (p, J = 6.9 Hz, 1H), 3.72 (s, 2H), 3.44 (s, 2H), 3.03 (dt, J = 8.0, 4.0 Hz, 1H), 1.82 (s, 2H), 1.61 (d, J = 6.9 Hz, 6H), 1.54 (s, 2H), 1.23 (s, 1H). MS: 505.3 |
| 1-(4-((4-amino-7- isopropyl-5-(4-(pyr- idin-2-ylamino)phen- yl)-7H-pyrrolo[2,3- d]pyrimidin-6-yl)- ethynyl)piperidin-1- yl)prop-2-en-1-one | 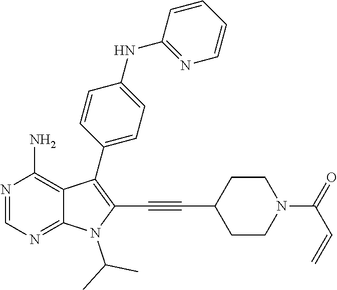 [SMILES: C=CC(=O)N1CCC(C#Cc2c(-c3ccc(Nc4ccccn4)cc3)c3c(N)ncnc3n2C(C)C)CC1] | ¹H NMR (300 MHz, DMSO) δ 9.20 (s, 1H), 8.24-8.12 (m, 2H), 7.82 (d, J = 8.5 Hz, 2H), 7.57 (ddd, J = 8.6, 7.2, 2.0 Hz, 1H), 7.46-7.36 (m, 2H), 6.88 (d, J = 8.4 Hz, 1H), 6.85-6.69 (m, 2H), 6.06 (dd, J = 16.7, 2.5 Hz, 1H), 5.63 (dd, J = 10.5, 2.4 Hz, 1H), 5.12 (p, J = 6.8 Hz, 1H), 3.88-3.67 (m, 2H), 3.40 (s, 1H), 3.33 (s, 1H), 3.04 (dt, J = 8.1, 4.1 Hz, 1H), 1.82 (s, 2H), 1.63 (d, J = 6.9 Hz, 6H), 1.59-1.46 (m, 2H). MS: 506.30 |
| 1-(4-((4-amino-7- isopropyl-5-(4-(6- methylpyridin-2-yl- amino)phenyl)-7H- pyrrolo[2,3-d]pyrim- idin-6-yl)ethynyl)- piperidin-1-yl)prop- 2-en-1-one | 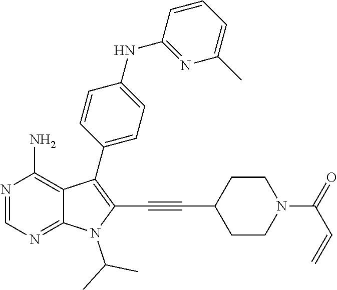 [SMILES: C=CC(=O)N1CCC(C#Cc2c(-c3ccc(Nc4cccc(C)n4)cc3)c3c(N)ncnc3n2C(C)C)CC1] | ¹H NMR (400 MHz, DMSO-d₆) δ 9.12 (s, 1H), 8.16 (s, 1H), 7.83-7.81 (m, 2H), 7.50-7.46 (m, 1H), 7.41- 7.39 (m, 2H), 6.77 (dd, J = 16.7, 10.5 Hz, 1H), 6.69 (d, J = 8.2 Hz, 1H), 6.65 (d, J = 7.3 Hz, 1H), 6.07 (dd, J = 16.7, 2.4 Hz, 1H), 5.64 (dd, J = 10.5, 2.5 Hz, 1H), 5.13 (p, J = 7.0 Hz, 1H), 3.78 (s, 1H), 3.70 (s, 1H), 3.42 (s, 2H), 3.04 (dt, J = 8.1, 4.0 Hz, 1H), 2.40 (s, 3H), 1.83 (s, 2H), 1.63 (d, J = 6.9 Hz, 6H), 1.55 (s, 2H). MS: 520.3 |
| 2-(4-(6-((1-acryloyl- piperidin-4-yl)eth- ynyl)-4-amino-7-iso- propyl-7H-pyrrolo [2,3-d]pyrimidin-5- yl)phenyl)-2-phen- ylacetonitrile | 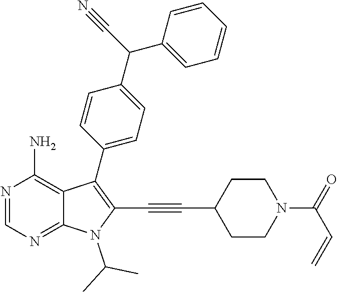 [SMILES: C=CC(=O)N1CCC(C#Cc2c(-c3ccc(C(C#N)c4ccccc4)cc3)c3c(N)ncnc3n2C(C)C)CC1] | ¹H NMR (400 MHz, DMSO-d₆) δ 8.17 (s, 1H), 7.56-7.40 (m, 8H), 7.43-7.32 (m, 1H), 6.78 (dd, J = 16.7, 10.5 Hz, 1H), 6.11 (dd, J = 16.7, 2.4 Hz, 1H), 5.99 (s, 1H), 5.89 (s, 1H), 5.69 (dd, J = 10.4, 2.4 Hz, 1H), 5.12 (p, J = 6.9 Hz, 1H), 3.67 (s, 2H), 3.31 (s, 2H), 3.01 (tt, J = 8.0, 4.0 Hz, 1H), 1.78 (s, 2H), 1.61 (d, J = 6.9 Hz, 6H), 1.49 (s, 2H). MS: 529 |
| 5-(6-((1-acryloyl-4- hydroxypiperidin-4- yl)ethynyl)-4-amino- 7-methyl-7H-pyr- rolo[2,3-d]pyrimidin- 5-yl)-2-phenoxy- benzonitrile | 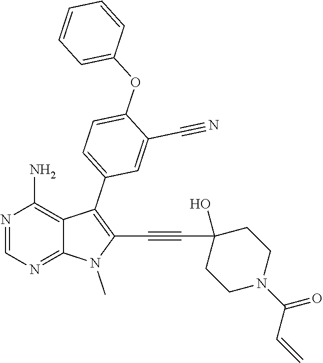 [SMILES: C=CC(=O)N1CCC(O)(C#Cc2c(-c3ccc(Oc4ccccc4)c(C#N)c3)c3c(N)ncnc3n2C)CC1] | ¹H NMR (400 MHz, DMSO-d6) δ 8.21 (s, 1H), 7.95 (s, 1H), 7.74 (d, J = 8.5 Hz, 1H), 7.50 (t, J = 7.7 Hz, 2H), 7.37-7.19 (m, 3H), 7.07 (d, J = 8.7 Hz, 1H), 6.79 (dd, J = 16.7, 10.5 Hz, 1H), 6.45 (s, 2H), 6.09 (d, J = 16.3 Hz, 1H), 5.93 (s, 1H), 5.76-5.61 (m, 1H), 3.96-3.89 (m, 1H), 3.83- 3.78 (m, 1H), 3.37 (s, 3H), 3.41-3.35 (m, 1H), 3.25-3.18 (m, 1H), 1.92- 1.85 (m, 2H), 1.70-1.61 (m, 2H). MS: 519.15 |
| 1-(4-((4-amino-5-(3- chloro-4-phenoxy- phenyl)-7-methyl- 7H-pyrrolo[2,3-d] pyrimidin-6-yl)eth- ynyl)-4-hydroxy- piperidin-1-yl)prop- 2-en-1-one | 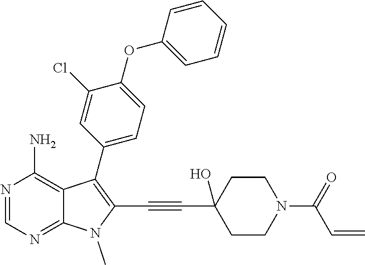 [SMILES: C=CC(=O)N1CCC(O)(C#Cc2c(-c3ccc(Oc4ccccc4)c(Cl)c3)c3c(N)ncnc3n2C)CC1] | ¹H NMR (400 MHz, DMSO-d₆) δ 8.27 (s, 1H), 7.71 (d, J = 2.1 Hz, 1H), 7.45-7.34 (m, 3H), 7.19-7.16 (m, 2H), 7.14-7.06 (m, 2H), 6.78 (dd, J = 16.7, 10.5 Hz, 1H), 6.41- 6.33 (m, 2H), 6.08 (dd, J = 16.7, 2.4 Hz, 1H), 5.97 (s, 1H), 5.66 (dd, J = 10.5, 2.4 Hz, 1H), 3.94 (s, 1H), 3.81 (s, 1H), 3.76 (s, 3H), 3.32 (s, 1H), 3.22 (s, 1H), 1.88 (s, 2H), 1.65 (s, 2H). MS: 528.15 |
| 1-(4-[2-[4-amino-5- (3-fluoro-4-phenoxy- phenyl)-7-methyl- 7H-pyrrolo[2,3-d] pyrimidin-6-yl]eth- ynyl]-4-hydroxy- piperidin-1-yl)prop- 2-en-1-one | 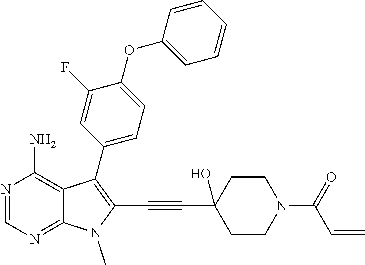 [SMILES: C=CC(=O)N1CCC(O)(C#Cc2c(-c3ccc(Oc4ccccc4)c(F)c3)c3c(N)ncnc3n2C)CC1] | ¹H NMR (400 MHz, DMSO-d6) δ 8.21 (s, 1H), 7.51-7.48 (dd, J = 11.9, 2.1 Hz, 1H), 7.43-7.38 (m, 2H), 7.35-7.32 (dd, 1H), 7.27-7.23(t, J = 8.5 Hz, 1H), 7.17-7.13 (m, 1H), 7.10-7.08 (m, 2H), 6.82-6.75 (dd, J = 16.7, 10.5 Hz, 1H), 6.4 (br, 2H), 6.11-6.06 (dd, J = 16.7, 2.4 Hz, 1H), 5.93 (s, 1H), 5.67-5.64 (dd, J = 10.5, 2.4 Hz, 1H), 3.93-3.90 (d, J = 12.5 Hz, 1H), 3.84-3.82 (d, J = 19.0 Hz, 1H), 3.75 (s, 3H), 3.36-3.32 (d, 1H), 3.26-3.23 (d, J = 14.5 Hz, 1H), 1.89-1.87 (d, 2H), 1.67-1.64 (d, J = 12.2 Hz, 2H). MS: 512.15 |
| 1-(4-(3-((4-amino-7- methyl-5-(3-methyl- 4-phenoxyphenyl)- 7H-pyrrolo[2,3-d] pyrimidin-6-yl)eth- ynyl)azetidin-1-yl)- piperidin-1-yl)prop- 2-en-1-one | 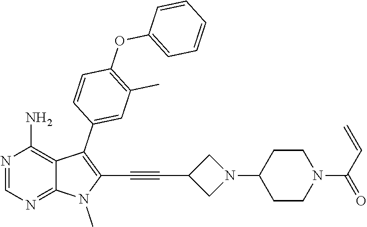 [SMILES: C=CC(=O)N1CCC(N2CC(C#Cc3c(-c4ccc(Oc5ccccc5)c(C)c4)c4c(N)ncnc4n3C)C2)CC1] | ¹H NMR (400 MHz, DMSO-d₆) δ 8.19 (s, 1H), 7.48 (d, J = 2.2 Hz, 1H), 7.43-7.30 (m, 3H), 7.11 (tt, J = 7.3, 1.1 Hz, 1H), 7.05-6.96 (m, 3H), 6.77 (dd, J = 16.7, 10.5 Hz, 1H), 6.24 (br, 1H), 6.06 (dd, J = 16.7, 2.4 Hz, 1H), 5.64 (dd, J = 10.5, 2.4 Hz, 1H), 3.96 (d, J = 13.1 Hz, 1H), 3.79 (d, J = 13.8 Hz, 1H), 3.72 (s, 3H), 3.56 (t, J = 7.0 Hz, 2H), 3.43 (p, J = 7.5 Hz, 1H), 3.13 (t, J = 11.7 Hz, 1H), 2.98 (q, J = 9.0, 7.9 Hz, 3H), 2.29-2.15 (m, 4H), 1.57 (s, 2H), 1.05 (s, 2H). MS: 547.25 |
| 5-(6-((1-(1-acryloyl- piperidin-4-yl)azeti- din-3-yl)ethynyl)-4- amino-7-methyl-7H- pyrrolo[2,3-d]pyrim- idin-5-y1)-2-phenoxy- benzonitrile | 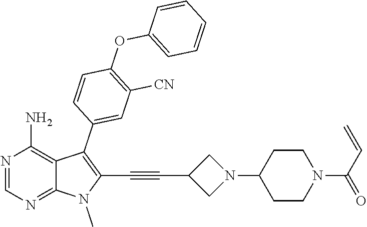 [SMILES: C=CC(=O)N1CCC(N2CC(C#Cc3c(-c4ccc(Oc5ccccc5)c(C#N)c4)c4c(N)ncnc4n3C)C2)CC1] | ¹H NMR (400 MHz, DMSO-d₆) δ 8.20 (s, 1H), 7.96 (d, J = 2.2 Hz, 1H), 7.74 (dd, J = 8.7, 2.3 Hz, 1H), 7.54-7.44 (m, 2H), 7.33-7.20 (m, 3H), 7.10 (d, J = 8.7 Hz, 1H), 6.78 (dd, J = 16.7, 10.5 Hz, 1H), 6.42 (s, 2H), 6.06 (dd, J = 16.7, 2.5 Hz, 1H), 5.64 (dd, J = 10.5, 2.5 Hz, 1H), 3.97 (d, J = 13.7 Hz, 1H), 3.81 (d, J = 13.6 Hz, 1H), 3.72 (s, 3H), 3.57 (t, J = 7.0 Hz, 2H), 3.44 (p, J = 7.5 Hz, 1H), 3.15 (t, J = 11.7 Hz, 1H), 3.05- 2.91 (m, 3H), 2.27 (dt, J = 8.7, 4.8 Hz, 1H), 1.59 (s, 2H), 1.06 (s, 2H). |
| | | MS: 558.20 |
| 1-(4-(3-((4-amino-5- (2-fluoro-4-phenoxy- phenyl)-7-methyl- 7H-pyrrolo[2,3-d] pyrimidin-6-yl)eth- ynyl)azetidin-1-yl)- piperidin-1-yl)prop- 2-en-1-one | 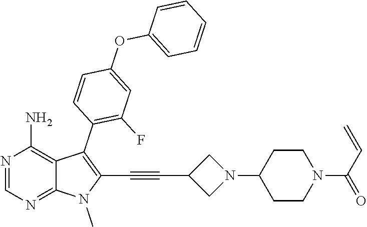 [SMILES: C=CC(=O)N1CCC(N2CC(C#Cc3c(-c4ccc(Oc5ccccc5)cc4F)c4c(N)ncnc4n3C)C2)CC1] | ¹H NMR (400 MHz, CDCl₃) δ 8.36 (s, 1H), 7.49-7.36 (m, 3H), 7.29- 7.20 (m, 1H), 7.18-7.10 (m, 2H), 6.92 (dd, J = 8.5, 2.5 Hz, 1H), 6.87 (dd, J = 10.8, 2.4 Hz, 1H), 6.58 (dd, J = 16.8, 10.6 Hz, 1H), 6.27 (dd, J = 16.8, 1.9 Hz, 1H), 5.69 (dd, J = 10.6, 2.0 Hz, 1H), 5.09 (s, 1H), 4.26 (d, J = 13.2 Hz, 1H), 3.86 (s, 4H), 3.72 (t, J = 7.0, 7.0 Hz, 2H), 3.48 (p, J = 7.8, 7.8, 7.4, 7.4 Hz, 1H), 3.20 (d, J = 12.7 Hz, 1H), 3.10 (d, J = 8.2 Hz, 3H), 2.39-2.30 (m, 1H), 1.72 (d, J = 13.2 Hz, 2H), 1.38-1.27 (m, |
| | | 2H). MS: 551.20 |
| 1-(4-(3-((4-amino-5- (2-methoxy-4-phen- oxyphenyl)-7-meth- yl-7H-pyrrolo[2,3-d] pyrimidin-6-yl)eth- ynyl)azetidin-1-yl)- piperidin-1-yl)prop- 2-en-1-one | 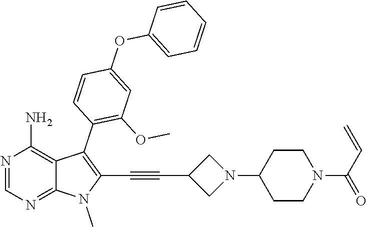 [SMILES: C=CC(=O)N1CCC(N2CC(C#Cc3c(-c4ccc(Oc5ccccc5)cc4OC)c4c(N)ncnc4n3C)C2)CC1] | ¹H NMR (400 MHz, DMSO-d6) δ 8.14 (s, 1H), 7.51-7.44 (m, 2H), 7.35 (d, J = 8.2 Hz, 1H), 7.22 (d, J = 7.5 Hz, 1H), 7.14-7.12 (m, 2H), 6.88 (d, J = 2.4 Hz, 1H), 6.77 (dd, J = 16.7, 10.5 Hz, 1H), 6.60 (dd, J = 8.3, 2.3 Hz, 1H), 6.06 (dd, J = 16.7, 2.5 Hz, 1H), 5.89 (br, 1H), 5.64 (dd, J = 10.5, 2.5 Hz, 1H), 3.94 (s, 1H), 3.77 (s, 1H), 3.71 (d, J = 10.4 Hz, 6H), 3.52 (s, 2H), 3.40 (q, J = 7.5 Hz, 1H), 3.17 (d, J = 13.3 Hz, 1H), 3.96- 2.90 (m, 3H), 2.22 (dd, J = 8.3, 4.3 Hz, 1H), 1.57 (s, 2H), 1.04 (s, 2H). |
| | | MS: 563.15 |
| 1-(4-(3-((4-amino-7- methyl-5-(4-(6-meth- ylpyridin-2-yloxy)- phenyl)-7H-pyrrolo [2,3-d]pyrimidin-6- yl)ethynyl)azetidin- 1-yl)piperidin-1-yl)- prop-2-en-1-one | 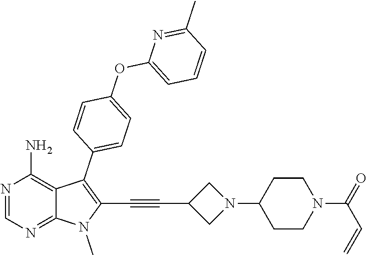 [SMILES: C=CC(=O)N1CCC(N2CC(C#Cc3c(-c4ccc(Oc5cccc(C)n5)cc4)c4c(N)ncnc4n3C)C2)CC1] | ¹H NMR (400 MHz, DMSO-d6) δ 8.20 (s, 1H), 7.76 (t, J = 7.8 Hz, 1H), 7.60-7.51 (m, 2H), 7.31-7.21(m, 2H), 7.04 (d, J = 7.3 Hz, 1H), 6.84 (d, J = 8.2 Hz, 1H), 6.77 (dd, J = 16.7, 10.5 Hz, 1H), 6.23 (s, 2H), 6.06 (dd, J = 16.7, 2.5 Hz, 1H), 5.64 (dd, J = 10.4, 2.5 Hz, 1H), 3.93 (d, J = 70.6 Hz, 3H), 3.74 (s, 3H), 3.55 (d, J = 49.5 Hz, 3H), 3.02 (d, J = 82.5 Hz, 4H), 2.36 (s, 3H), 1.63 (s, 2H), 1.06 (d, J = 15.1 Hz, 2H). MS: 548.25 |

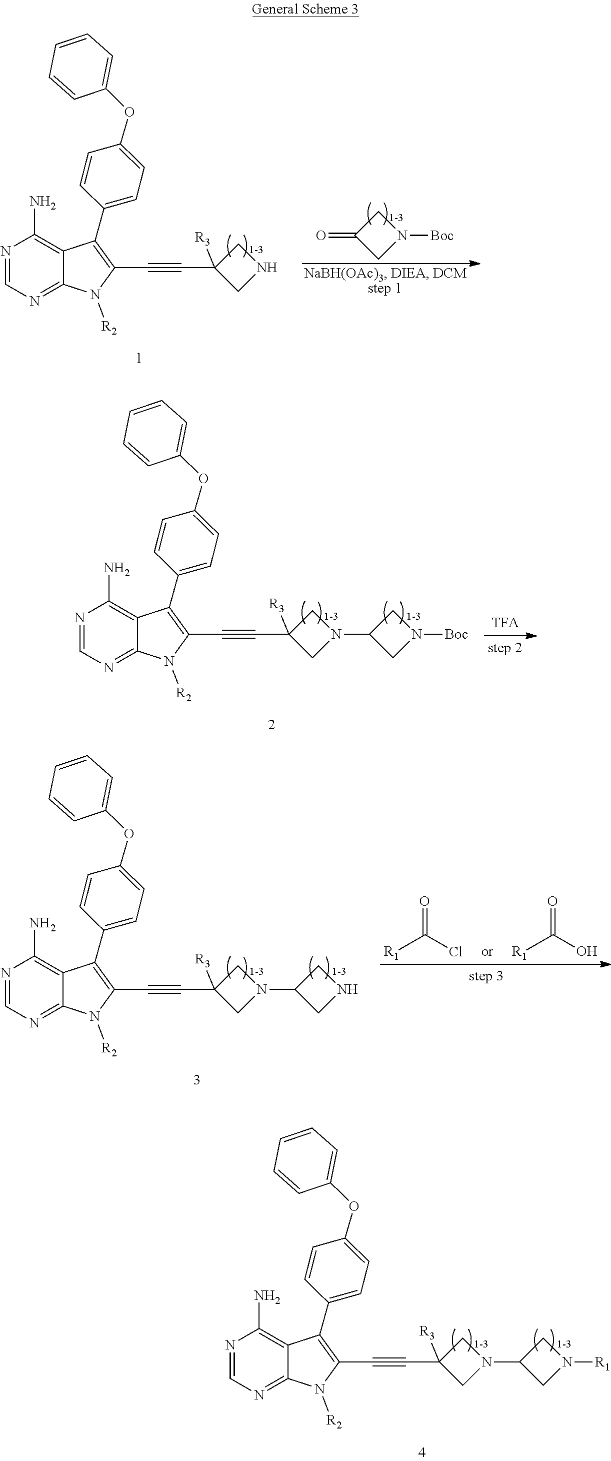

### Example 8

### 1-(3-(4-((4-amino-7-isopropyl-5-(4-phenoxyphenyl)-7H-pyrrolo[2,3-d]pyrimidin-6-yl)ethynyl)piperidin-1-yl)azetidin-1-yl)prop-2-en-1-one

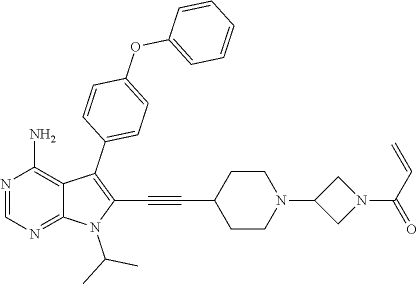 [SMILES: C=CC(=O)N1CC(N2CCC(C#Cc3c(-c4ccc(Oc5ccccc5)cc4)c4c(N)ncnc4n3C(C)C)CC2)C1]

### tert-butyl-3-(4-((4-amino-7-isopropyl-5-(4-phenoxyphenyl)-7H-pyrrolo[2,3-d]pyrimidin-6-yl)ethynyl)piperidin-1-yl)azetidine-1-carboxylate

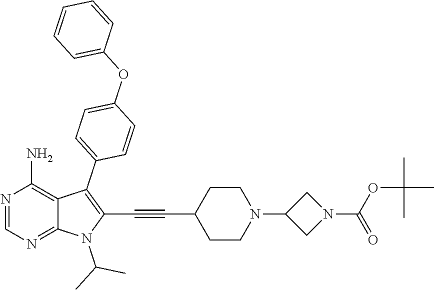 [SMILES: CC(C)n1c(C#CC2CCN(C3CN(C(=O)OC(C)(C)C)C3)CC2)c(-c2ccc(Oc3ccccc3)cc2)c2c(N)ncnc21]

### Step 1

A resealable reaction vial was charged with 7-isopropyl-5-(4-phenoxyphenyl)-6-(piperidin-4-ylethynyl)-7H-pyrrolo[2,3-d]pyrimidin-4-amine (500 mg, 1.10 mmol), tert-butyl 3-oxoazetidine-1-carboxylate (376 mg, 2.20 mmol), NaBH(OAc)₃ (466 mg, 2.20 mmol), DCM (10 mL), DIEA (425 mg, 3.30 mmol), and a stirbar before being evacuated and purged with nitrogen three times. The mixture was stirred for 1 h at room temperature. The reaction mixture was diluted with H₂O (50 mL), and the aqueous phase was extracted with EA (100 mL) three times. The combined organic layers were washed with saturated brines, dried over sodium sulfate, filtered, and concentrated in vacuo. The resulting crude material was purified by silica gel chromatography (PE/EA=3/2). Concentration in vacuo resulted in tert-butyl-3-(4-((4-amino-7-isopropyl-5-(4-phenoxyphenyl)-7H-pyrrolo[2,3-d]pyrimidin-6-yl)ethynyl)piperidin-1-yl)azetidine-1-carboxylate (450 mg,) as a yellow amorphous solid.

### 6-((1-(azetidin-3-yl)piperidin-4-yl)ethynyl)-7-isopropyl-5-(4-phenoxyphenyl)-7H-pyrrolo[2,3-d]pyrimidin-4-amine

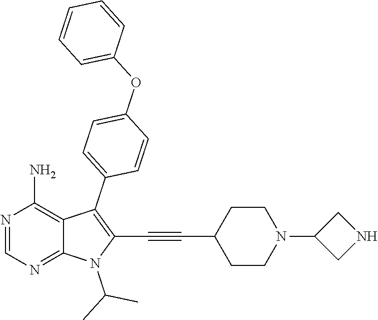 [SMILES: CC(C)n1c(C#CC2CCN(C3CNC3)CC2)c(-c2ccc(Oc3ccccc3)cc2)c2c(N)ncnc21]

### Step 2

A round bottomed flask was charged with tert-butyl-3-(4-((4-amino-7-isopropyl-5-(4-phenoxyphenyl)-7H-pyrrolo[2,3-d]pyrimidin-6-yl)ethynyl)piperidin-1-yl)azetidine-1-carboxylate (500 mg, 725 μmol) and a stirbar. DCM (10 mL) and TFA (2 mL) was added, and the solution was stirred for 1 h at room temperature. The reaction mixture was diluted with H₂O (20 mL), and the pH of the solution was adjusted to ˜9-10 with Na₂CO₃. The aqueous phase was extracted with EA (100 mL) three times. The combined organic layers were washed with saturated brines, dried over sodium sulfate, filtered, and concentrated in vacuo to get 6-((1-(azetidin-3-yl)piperidin-4-yl)ethynyl)-7-isopropyl-5-(4-phenoxyphenyl)-7H-pyrrolo[2,3-d]pyrimidin-4-amine (380 mg,) as a yellow oil.

### 1-(3-(4-((4-amino-7-isopropyl-5-(4-phenoxyphenyl)-7H-pyrrolo[2,3-d]pyrimidin-6-yl)ethynyl)piperidin-1-yl)azetidin-1-yl)prop-2-en-1-one

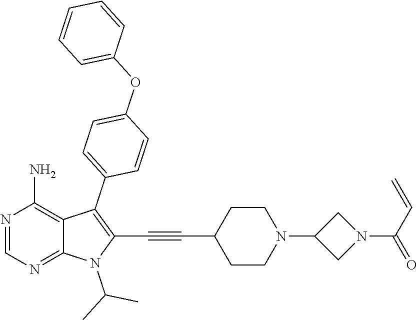 [SMILES: C=CC(=O)N1CC(N2CCC(C#Cc3c(-c4ccc(Oc5ccccc5)cc4)c4c(N)ncnc4n3C(C)C)CC2)C1]

### Step 3

A resealable reaction vial was charged with 6-((1-(azetidin-3-yl)piperidin-4-yl)ethynyl)-7-isopropyl-5-(4-phenoxyphenyl)-7H-pyrrolo[2,3-d]pyrimidin-4-amine (50 mg, 98.6 μmol), DCM (3 mL), TEA (29.7 mg, 295 μmol) and a stirbar before being evacuated and purged with nitrogen three times. Then the mixture was cooled to 0° C., and acryloyl chloride (8.92 mg, 98.6 μmol) was added, the mixture was stirred for 1 h at room temperature. The reaction mixture was diluted with MeOH (1 mL) and concentrated in vacuo. The resulting crude material was purified by HPLC. Column: XBridge Prep OBD C18 Column 30×150 mm 5 um; Mobile Phase A:Water (10 MMOL/L NH₄HCO₃+0.1% NH₃·H₂O), Mobile Phase B: ACN; Flow rate: 60 mL/min; Gradient: 48% B to 49% B in 8 min; 254/220 nm; Rt: 6.30 min. Lyophilization yielded 1-(3-(4-((4-amino-7-isopropyl-5-(4-phenoxyphenyl)-7H-pyrrolo[2,3-d]pyrimidin-6-yl)ethynyl)piperidin-1-yl)azetidin-1-yl)prop-2-en-1-one (19.0 mg, 33.8 μmol) as a white amorphous solid.

Compounds prepared by substantially the same methods, and their characterization data, are listed in Table 7, below.

TABLE 7
Selected Compounds

| | | Proton NMR & |
|---|---|---|
| Compound | Structure | Mass Spec. (obs.) |
| 1-(3-(4-((4-amino-7- isopropyl-5-(4- phenoxyphenyl)-7H- pyrrolo[2,3- d]pyrimidin-6- yl)ethynyl)piperidin- 1-yl)azetidin-1- yl)prop-2-en-1-one | 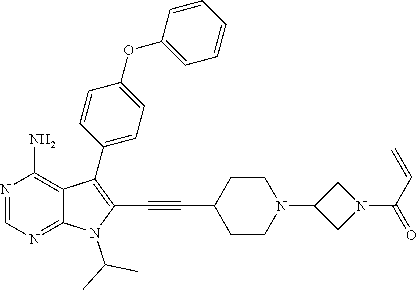 [SMILES: C=CC(=O)N1CC(N2CCC(C#Cc3c(-c4ccc(Oc5ccccc5)cc4)c4c(N)ncnc4n3C(C)C)CC2)C1] | ¹H NMR (300 MHz, DMSO-d₆) δ 8.15 (s, 1H), 7.51 − 7.40 (m, 4H), 7.11 (dd, J = 21.6, 7.9 Hz, 5H), 6.28 (dd, J = 17.0, 10.2 Hz, 1H), 6.05 (dd, J = 17.0, 2.4 Hz, 2H), 5.66 (dd, J = 10.2, 2.4 Hz, 1H), 5.13 (t, J = 6.8 Hz, 1H), 4.19 (t, J = 8.0 Hz, 1H), 4.0 − 3.86 (m, 2H), 3.69 (dd, J = 10.5, 5.0 Hz, 1H), 3.06 (s, 1H), 2.77 (s, 1H), 2.45 (s, 2H), 2.13 (s, 2H), 1.80 (s, 2H), 1.62 (d, J = 6.9 Hz, 6H), 1.55 (s, 2H). MS: 561.2 |
| 7-isopropyl-5-(4- phenoxyphenyl)-6- ((1-(1- (vinylsulfonyl) azetidin-3- yl)piperidin-4- yl)ethynyl)-7H- pyrrolo[2,3- d]pyrimidin-4-amine | 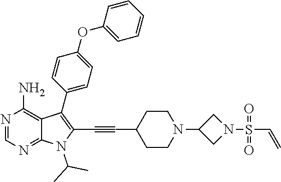 [SMILES: C=CS(=O)(=O)N1CC(N2CCC(C#Cc3c(-c4ccc(Oc5ccccc5)cc4)c4c(N)ncnc4n3C(C)C)CC2)C1] | ¹H NMR (400 MHz, DMSO-d₆) δ 8.15 (s, 1H), 7.50 − 7.41 (m, 4H), 7.18 (ddt, J = 8.4, 7.2, 1.1 Hz, 1H), 7.13 − 7.09 (m, 4H), 7.00 (dd, J = 16.5, 10.0 Hz, 1H), 6.30 (d, J = 10.0 Hz, 1H), 6.18 (d, J = 16.6 Hz, 1H), 5.12 (p, J = 6.9 Hz, 1H), 3.75 (dd, J = 8.3, 7.1 Hz, 2H), 3.63 (dd, J = 8.3, 6.3 Hz, 2H), 3.06 (p, J = 6.6 Hz, 1H), 2.76 (s, 1H), 2.40 (s, 2H), 2.10 (s, 2H), 1.81 (d, J = 11.1 Hz, 2H), 1.62 (d, J = 6.9 Hz, 6H), 1.55 (s, 2H). MS: 597.2 |
| (E)-1-(3-(4-((4- amino-7-isopropyl- 5-(4- phenoxyphenyl)-7H- pyrrolo[2,3- d]pyrimidin-6- yl)ethynyl)piperidin- 1-yl)azetidin-1-yl)- 4- (dimethylamino)but- 2-en-1-one | 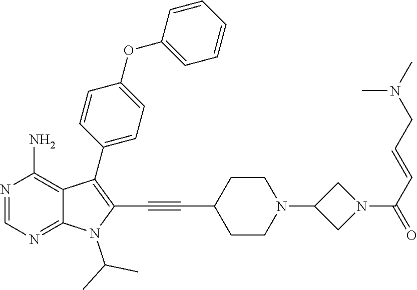 [SMILES: CC(C)n1c(C#CC2CCN(C3CN(C(=O)/C=C/CN(C)C)C3)CC2)c(-c2ccc(Oc3ccccc3)cc2)c2c(N)ncnc21] | ¹H NMR (400 MHz, DMSO-d₆) δ 8.16 (s, 1H), 7.50 (d, J = 8.2 Hz, 2H), 7.42 (t, J = 7.7 Hz, 2H), 7.17 (t, J = 7.4 Hz, 1H), 7.12 (d, J = 8.1 Hz, 4H), 6.57 (dt, J = 13.7, 6.2 Hz, 1H), 6.08 (d, J = 15.4 Hz, 1H), 5.14 − 5.11 (m, 1H), 4.15 (t, J = 8.2 Hz, 1H), 3.95 (s, 1H), 3.88 (t, J = 8.8 Hz, 1H), 3.67 (dd, J = 10.0, 5.1 Hz, 1H), 3.06 − 3.00 (m, 3H), 2.78 (s, 1H), 2.43 (s, 2H), 2.14 (s, 8H), 1.81 (s, 2H), 1.62 (d, J = 6.9 Hz, 8H). MS: 618.5 |
| (E)-1-(3-(4-((4- amino-7-isopropyl- 5-(4- phenoxyphenyl)-7H- pyrrolo[2,3- d]pyrimidin-6- yl)ethynyl)piperidin- 1-yl)azetidin-1- yl)but-2-en-1-one | 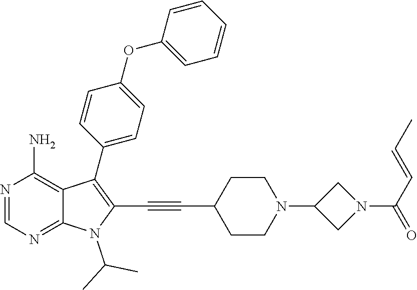 [SMILES: C/C=C/C(=O)N1CC(N2CCC(C#Cc3c(-c4ccc(Oc5ccccc5)cc4)c4c(N)ncnc4n3C(C)C)CC2)C1] | ¹H NMR (400 MHz, DMSO-d₆) δ 8.16 (s, 1H), 7.52 − 7.46 (m, 4H), 7.12 − 7.09 (m, 5H), 6.71 − 6.57 (m, 1H), 5.97 (dd, J = 15.2, 1.8 Hz, 1H), 5.12 (p, J = 6.9 Hz, 1H), 4.14 (t, J = 7.9 Hz, 1H), 3.96 − 3.81 (m, 2H), 3.65 (dd, J = 10.1, 5.2 Hz, 1H), 3.03 (q, J = 6.8, 6.1 Hz, 1H), 2.77 (s, 1H), 2.45 (s, 2H), 2.12 (s, 2H), 1.82 (dd, J = 6.8, 1.7 Hz, 5H), 1.62 (d, J = 6.9 Hz, 6H), 1.56 (s, 2H). MS: 575.2 |
| 1-(4-(3-((4-amino-7- isopropyl-5-(4- phenoxyphenyl)-7H- pyrrolo[2,3- d]pyrimidin-6- yl)ethynyl)azetidin- 1-yl)piperidin-1- yl)prop-2-en-1-one | 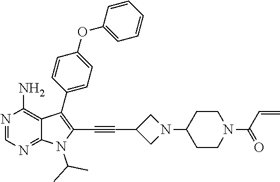 [SMILES: C=CC(=O)N1CCC(N2CC(C#Cc3c(-c4ccc(Oc5ccccc5)cc4)c4c(N)ncnc4n3C(C)C)C2)CC1] | ¹H NMR (400 MHz, DMSO-d₆) δ 8.17 (s, 1H), 7.56 − 7.47 (m, 2H), 7.48 − 7.38 (m, 2H), 7.23 − 7.08 (m, 5H), 6.78 (dd, J = 16.7, 10.4 Hz, 1H), 6.07 (dd, J = 16.7, 2.4 Hz, 1H), 5.65 (dd, J = 10.4, 2.5 Hz, 1H), 5.10 (p, J = 6.8 Hz, 1H), 3.93 (s, 1H), 3.79 (d, J = 13.3 Hz, 1H), 3.53 (s, 2H), 3.40 (t, J = 7.4 Hz, 1H), 3.16 (t, J = 11.4 Hz, 1H), 3.0 (s, 3H), 2.22 (s, 1H), 1.62 (d, J = 6.9 Hz, 8H), 1.06 (s, 2H). MS: 561.30 |
| 1-(4-(3-((4-amino-7- methyl-5-(4- phenoxyphenyl)-7H- pyrrolo[2,3- d]pyrimidin-6- yl)ethynyl)azetidin- 1-yl)piperidin-1- yl)prop-2-en-1-one | 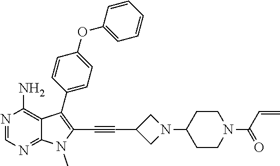 [SMILES: C=CC(=O)N1CCC(N2CC(C#Cc3c(-c4ccc(Oc5ccccc5)cc4)c4c(N)ncnc4n3C)C2)CC1] | ¹H NMR (400 MHz, DMSO-d₆) δ 8.19 (s, 1H), 7.52 (d, J = 8.1 Hz, 2H), 7.43 (t, J = 7.7 Hz, 2H), 7.20 − 7.12 (m, 5H), 6.78 (dd, J = 16.8, 10.5 Hz, 1H), 6.23-6.13 (br, 1H), 6.07 (dd, J = 16.6, 2.2 Hz, 1H), 5.65 (dd, J = 10.8, 2.1 Hz, 1H), 3.96 (d, J = 12.9 Hz, 1H), 3.80 (d, J = 13.7 Hz, 1H), 3.72 (s, 3H), 3.56 (t, J = 6.9 Hz, 2H), 3.42 (p, J = 7.2 Hz, 1H), 3.16 (t, J = 10.5 Hz, 1H), 3.00 (t, J = 7.1 Hz, 3H), 2.22 (s, 1H), 1.58 (s, 2H), 1.05 (s, 2H). MS: 533.20 |
| 1-(4-(3-((4-amino-7- methyl-5-(4- phenoxyphenyl)-7H- pyrrolo[2,3- d]pyrimidin-6- yl)ethynyl)-3- hydroxyazetidin-1- yl)piperidin-1- yl)prop-2-en-1-one | 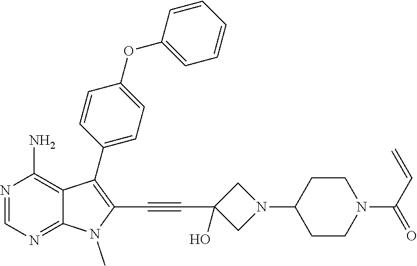 [SMILES: C=CC(=O)N1CCC(N2CC(O)(C#Cc3c(-c4ccc(Oc5ccccc5)cc4)c4c(N)ncnc4n3C)C2)CC1] | ¹H NMR (400 MHz, DMSO-d₆) δ 8.20 (s, 1H), 7.54 (d, J = 8.5 Hz, 2H), 7.43 (t, J = 7.8 Hz, 2H), 7.18 (t, J = 7.4 Hz, 1H), 7.11 (dd, J = 8.2, 6.0 Hz, 4H), 6.77 (dd, J = 16.7, 10.5 Hz, 1H), 6.29 (s, 1H), 6.06 (dd, J = 16.7, 2.4 Hz, 1H), 5.64 (dd, J = 10.4, 2.4 Hz, 1H), 4.00 (d, J = 12.6 Hz, 1H), 3.82 (d, J = 13.6 Hz, 1H), 3.74 (s, 3H), 3.52 (d, J = 5.6 Hz, 2H), 3.14 (d, J = 7.1 Hz, 3H), 2.95 (t, J = 11.4 Hz, 1H), 2.27 (d, J = 9.2 Hz, 1H), 1.60 (s, 2H), 1.06 (s, 2H). MS: 549.25 |
| 1-(4-(3-((4-amino-7- isopropyl-5-(4- phenoxyphenyl)-7H- pyrrolo[2,3- d]pyrimidin-6- yl)ethynyl)-3- hydroxyazetidin-1- yl)piperidin-1- yl)prop-2-en-1-one | 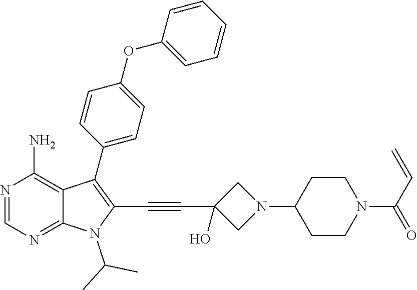 [SMILES: C=CC(=O)N1CCC(N2CC(O)(C#Cc3c(-c4ccc(Oc5ccccc5)cc4)c4c(N)ncnc4n3C(C)C)C2)CC1] | ¹H NMR (400 MHz, DMSO-d₆) δ 8.18 (s, 1H), 7.53 (d, J = 8.6 Hz, 2H), 7.43 (t, J = 7.9 Hz, 2H), 7.19 (t, J = 7.4 Hz, 1H), 7.10 (t, J = 8.7 Hz, 4H), 6.77 (dd, J = 16.7, 10.4 Hz, 1H), 6.25 (s, 1H), 6.08-6.03 (dd, J = 16.7, 2.5 Hz, 1H), 5.66- 5.63 (dd, J = 10.4, 2.4 Hz, 1H), 5.18-5.13 (t, J = 6.9 Hz, 1H), 3.94 (t, 1H), 3.78 (m, 1H), 3.50 (s, 2H), 3.20 − 3.09 (m, 3H), 3.01 (m, 1H), 2.28 (m, 1H), 1.63 (d, J = 6.9 Hz, 8H), 1.08 (s, 2H). MS: 577.35 |
| 1-((3R,4S)-4-(3-((4- amino-7-methyl-5- (4-phenoxyphenyl)- 7H-pyrrolo[2,3- d]pyrimidin-6- yl)ethynyl)azetidin- 1-yl)-3- methylpiperidin-1- yl)prop-2-en-1-one | 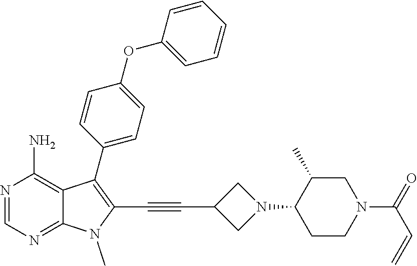 [SMILES: C=CC(=O)N1CC[C@H](N2CC(C#Cc3c(-c4ccc(Oc5ccccc5)cc4)c4c(N)ncnc4n3C)C2)[C@H](C)C1] | ¹H NMR (400 MHz, DMSO-d₆) δ 8.19 (s, 1H), 7.56 − 7.48 (m, 2H), 7.43 (t, J = 7.9 Hz, 2H), 7.23 − 7.09 (m, 5H), 6.78 (dd, J = 16.3, 9.7 Hz, 1H), 6.07 (t, J = 15.5 Hz, 1H), 5.65 (d, J = 11.2 Hz, 1H), 4.39 − 4.16 (m, 1H), 3.97 − 3.82 (m, 1H), 3.72 (s, 3H), 3.53 (q, J = 6.1 Hz, 2H), 3.42 (p, J = 7.5 Hz, 1H), 3.14 − 2.93 (m, 3H), 2.78 − 2.53 (m, 1H), 2.28 − 2.20 (m, 1H), 1.82 (s, 1H), 1.40 (d, J = 13.0 Hz, 1H), 1.28 − 1.02 (m, 1H), 0.70 (d, J = 7.0 Hz, 3H). MS: 547.30 |
| 1-((3R,4R)-4-(3-((4- amino-7-methyl-5- (4-phenoxyphenyl)- 7H-pyrrolo[2,3- d]pyrimidin-6- yl)ethynyl)azetidin- 1-yl)-3- methylpiperidin-1- yl)prop-2-en-1-one | 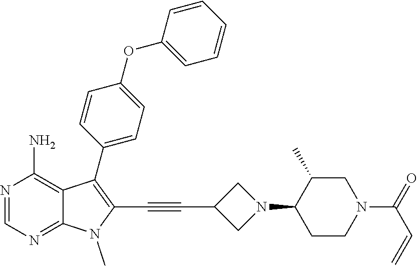 [SMILES: C=CC(=O)N1CC[C@@H](N2CC(C#Cc3c(-c4ccc(Oc5ccccc5)cc4)c4c(N)ncnc4n3C)C2)[C@H](C)C1] | ¹H NMR (400 MHz, DMSO-d₆) δ 8.19 (s, 1H), 7.56 − 7.48 (m, 2H), 7.43 (t, J = 7.9 Hz, 2H), 7.23 − 7.10 (m, 5H), 6.78 (dd, J = 16.3, 9.7 Hz, 1H), 6.07 (t, J = 15.5 Hz, 1H), 5.65 (d, J = 11.2 Hz, 1H), 4.39 − 4.16 (m, 1H), 3.97 − 3.82 (m, 1H), 3.72 (s, 3H), 3.53 (q, J = 6.1 Hz, 2H), 3.42 (p, J = 7.5 Hz, 1H), 3.14 − 2.93 (m, 3H), 2.78 − 2.53 (m, 1H), 2.28 − 2.20 (m, 1H), 1.81 (s, 1H), 1.40 (d, J = 13.0 Hz, 1H), 1.28 − 1.02 (m, 1H), 0.70 (d, J = 7.0 Hz, 3H). MS: 547.30 |
| 1-((3S,4S)-4-(3-((4- amino-7-methyl-5- (4-phenoxyphenyl)- 7H-pyrrolo[2,3- d]pyrimidin-6- yl)ethynyl)azetidin- 1-yl)-3- methylpiperidin-1- yl)prop-2-en-1-one | 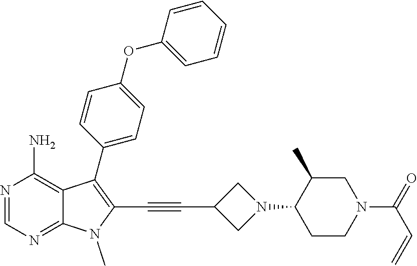 [SMILES: C=CC(=O)N1CC[C@H](N2CC(C#Cc3c(-c4ccc(Oc5ccccc5)cc4)c4c(N)ncnc4n3C)C2)[C@@H](C)C1] | ¹H NMR (400 MHz, DMSO-d₆) δ 8.19 (s, 1H), 7.57 − 7.48 (m, 2H), 7.48 − 7.37 (m, 2H), 7.23 − 7.07 (m, 5H), 6.83 − 6.71 (m, 1H), 6.21 (s, 2H), 6.13 − 6.02 (m, 1H), 5.64 (dd, J = 10.4, 2.5 Hz, 1H), 3.72 (s, 3H), 3.62 (t, J = 6.7 Hz, 1H), 3.53 (d, J = 12.2 Hz, 2H), 3.43 (q, J = 7.6 Hz, 2H), 3.32 (s, 1H), 3.09-3.01 (m, 2H), 2.06 (q, J = 4.7 Hz, 1H), 1.69 − 1.46 (m, 2H), 1.26 − 1.17 (m, 1H), 0.85 (d, J = 6.9 Hz, 3H). MS: 547.25 |
| 1-(4-(3-((4-amino-7- (1-hydroxypropan-2- yl)-5-(4- phenoxyphenyl)-7H- pyrrolo[2,3- d]pyrimidin-6- yl)ethynyl)azetidin- 1-yl)piperidin-1- yl)prop-2-en-1-one | 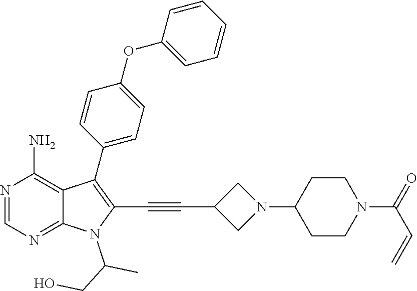 [SMILES: C=CC(=O)N1CCC(N2CC(C#Cc3c(-c4ccc(Oc5ccccc5)cc4)c4c(N)ncnc4n3C(C)CO)C2)CC1] | ¹H NMR (400 MHz, DMSO-d₆) δ 8.16 (s, 1H), 7.53 − 7.51 (m, 2H), 7.43 (t, J = 7.8 Hz, 2H), 7.18 (t, J = 7.4 Hz, 1H), 7.12 (dd, J = 8.2, 4.5 Hz, 4H), 6.77 (dd, J = 16.7, 10.5 Hz, 1H), 6.07 (dd, J = 16.7, 2.5 Hz, 1H), 5.64 (dd, J = 10.5, 2.5 Hz, 1H), 5.02 (t, J = 5.7 Hz, 1H), 4.89 (h, J = 7.1 Hz, 1H), 4.07 (ddd, J = 10.9, 8.0, 6.1 Hz, 1H), 3.94 (d, J = 13.1 Hz, 1H), 3.90 − 3.75 (m, 2H), 3.53 (t, J = 6.9 Hz, 2H), 3.39 (t, J = 7.4 Hz, 1H), 3.16 (t, J = 11.4 Hz, 1H), 2.98 (q, J = 9.5, 6.5 Hz, 3H), 2.22 (tt, J = 8.2, 3.6 Hz, 1H), 1.63 − 1.50 (m, 5H), 1.06 (t, J = 11.9 Hz, 2H). MS: 577.30 |
| 1-(4-(3-((4-amino-7- methyl-5-(4- phenoxyphenyl)-7H- pyrrolo[2,3- d]pyrimidin-6- yl)ethynyl)azetidin- 1-yl)-3- methylpiperidin-1- yl)prop-2-en-1-one | 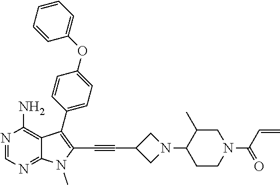 [SMILES: C=CC(=O)N1CCC(N2CC(C#Cc3c(-c4ccc(Oc5ccccc5)cc4)c4c(N)ncnc4n3C)C2)C(C)C1] | ¹H NMR (400 MHz, DMSO-d₆) δ 8.20 (s, 1H), 8.14 (s, 0.52H), 7.57 − 7.49 (m, 2H), 7.48 − 7.38 (m, 2H), 7.23 − 7.09 (m, 5H), 6.79 (dd, J = 17.4, 9.5 Hz, 1H), 6.09 (t, J = 14.5 Hz, 1H), 5.70 − 5.62 (m, 1H), 4.43-4.15 (m, 1H), 4.02-3.70 (m, 5H), 3.70-3.40 (m, 2H), 3.12 (d, J = 13.8 Hz, 1H), 3.03 − 2.96 (m, 1H), 2.74 (d, J = 13.1 Hz, 1H), 2.56 (d, J = 9.8 Hz, 1H), 1.86 (s, 1H), 1.54 (s, 1H), 1.44 (s, 1H), 0.88 (s, 1H), 0.72 (d, J = 6.9 Hz, 3H). MS: 547.30 |
| 1-((2S)-4-(3-((4- amino-7-methyl-5- (4-phenoxyphenyl)- 7H-pyrrolo[2,3- d]pyrimidin-6- yl)ethynyl)azetidin- 1-yl)-2- (hydroxymethyl) piperidin- 1-yl)prop-2-en- 1-one | 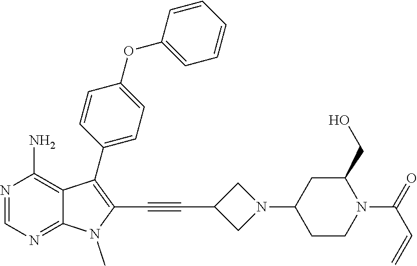 [SMILES: C=CC(=O)N1CCC(N2CC(C#Cc3c(-c4ccc(Oc5ccccc5)cc4)c4c(N)ncnc4n3C)C2)C[C@H]1CO] | ¹H NMR (400 MHz, DMSO-d₆) δ 8.18 (s, 1H), 7.56 − 7.47 (m, 2H), 7.47 − 7.37 (m, 2H), 7.22 − 7.13 (m, 1H), 7.15 − 7.08 (m, 4H), 6.77 (dd, J = 16.8, 10.5 Hz, 1H), 6.40 − 6.11 (br, 1H), 6.04 (dd, J = 16.7, 2.5 Hz, 1H), 5.63 − 5.55 (m, 1H), 4.88 (s, 1H), 4.53 − 3.65 (m, 6H), 3.54 (t, J = 7.1 Hz, 3H), 3.39 (q, J = 7.2 Hz, 1H), 3.03- 3.01 (m, 1H), 3.00-2.98 (m, 1H), 2.91- 2.74 (br, 1H), 2.38 (s, 1H), 1.63 − 1.60 (m, 1H), 1.56 − 1.45 (m, 2H), 1.36 (s, 1H). MS: 563.30 |
| 1-(4-(3-((4-amino-7- methyl-5-(4- phenoxyphenyl)-7H- pyrrolo[2,3- d]pyrimidin-6- yl)ethynyl)azetidin- 1-yl)piperidin-1-yl)- 2-methylprop-2-en- 1-one | 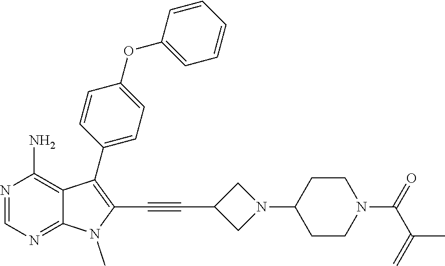 [SMILES: C=C(C)C(=O)N1CCC(N2CC(C#Cc3c(-c4ccc(Oc5ccccc5)cc4)c4c(N)ncnc4n3C)C2)CC1] | ¹H NMR (400 MHz, DMSO-d₆) δ 8.19 (s, 1H), 7.53 (d, J = 8.3 Hz, 2H), 7.43 (t, J = 7.8 Hz, 2H), 7.23 − 7.10 (m, 5H), 6.23 (s, 1H), 5.13 (s, 1H), 4.92 (s, 1H), 3.98 − 3.80 (m, 1H), 3.72 (s, 3H), 3.75 − 3.62 (m, 1H), 3.55 (t, J = 6.6 Hz, 2H), 3.48 − 3.79 (m, 1H), 3.16 − 2.88 (m, 4H), 2.25 (s, 1H), 1.83 (s, 3H), 1.59 (d, J = 12.8 Hz, 2H), 1.06 (d, J = 11.1 Hz, 2H). MS: 547.30 |
| (R)-1-(4-(3-((4- amino-5-(4- phenoxyphenyl)-7- (tetrahydrofuran-3- yl)-7H-pyrrolo[2,3- d]pyrimidin-6- yl)ethynyl)azetidin- 1-yl)piperidin-1- yl)prop-2-en-1-one | 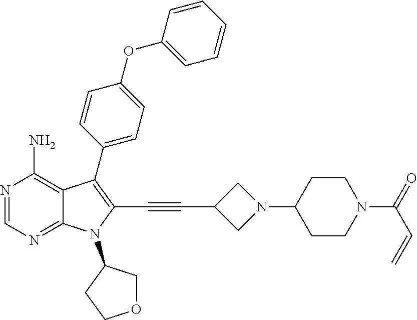 [SMILES: C=CC(=O)N1CCC(N2CC(C#Cc3c(-c4ccc(Oc5ccccc5)cc4)c4c(N)ncnc4n3[C@@H]3CCOC3)C2)CC1] | ¹H NMR (400 MHz, DMSO-d₆) δ 8.19 (s, 1H), 7.52 − 7.50 (m, 2H), 7.43 (t, J = 7.8 Hz, 2H), 7.18 (t, J = 7.4 Hz, 1H), 7.13 − 7.11 (m, 4H), 6.78 (dd, J = 16.7, 10.5 Hz, 1H), 6.40-6.20 (br, 1H), 6.08 (dd, J = 16.7, 2.5 Hz, 1H), 5.64 (dd, J = 10.5, 2.4 Hz, 1H), 5.51 (p, J = 7.7 Hz, 1H), 4.22 (q, J = 8.0 Hz, 1H), 4.08 − 4.05 (m, 2H), 3.93 (q, J = 7.6 Hz, 2H), 3.79 (d, J = 13.2 Hz, 1H), 3.53 (t, J = 7.0 Hz, 2H), 3.39 (p, J = 7.5 Hz, 1H), 3.16 (m, 1H), 2.99 (m, 3H), 2.62 (dt, J = 14.3, 7.0 Hz, 1H), 2.36 (m, J = 5.1 Hz, 1H), 2.23 (m, 1H), 1.59 (m, 2H), 1.06 (m, 2H). MS: 589.30 |
| (S)-1-(4-(3-((4- amino-5-(4- phenoxyphenyl)-7- (tetrahydrofuran-3- yl)-7H-pyrrolo[2,3- d]pyrimidin-6- yl)ethynyl)azetidin- 1-yl)piperidin-1- yl)prop-2-en-1-one | 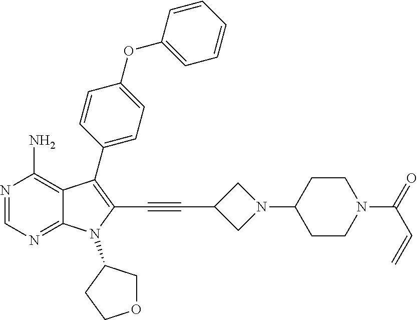 [SMILES: C=CC(=O)N1CCC(N2CC(C#Cc3c(-c4ccc(Oc5ccccc5)cc4)c4c(N)ncnc4n3[C@H]3CCOC3)C2)CC1] | ¹H NMR (400 MHz, DMSO-d₆) δ 8.19 (s, 1H), 7.52 − 7.50 (m, 2H), 7.43 (t, 2H), 7.19 (t, J = 7.4 Hz, 1H), 7.13 − 7.11 (m, 4H), 6.78 (dd, J = 16.7, 10.5 Hz, 1H), 6.40-6.20 (br, 1H), 6.08-6.04 (dd, J = 16.7, 2.4 Hz, 1H), 5.65 (dd, J = 10.5, 2.5 Hz, 1H), 5.53 − 5.49 (m, 1H), 4.22 − 4.21 (m, 1H), 4.08 − 4.05 (m, 2H), 3.94 (q, J = 7.5 Hz, 2H), 3.80 (d, J = 12.8 Hz, 1H), 3.53 (t, J = 7.0 Hz, 2H), 3.40 (q, J = 7.4 Hz, 1H), 3.16 (m, 1H), 3.01 (d, J = 7.9 Hz, 3H), 2.62 (dt, J = 14.1, 6.8 Hz, 1H), 2.37 (m, 1H), 2.22 (m, 1H), 1.56 (m, 2H), 1.06 (m, 2H). MS: 589.30 |
| 1-((3R,4R)-4-(3-((4- amino-7-methyl-5- (4-phenoxyphenyl)- 7H-pyrrolo[2,3- d]pyrimidin-6- yl)ethynyl)azetidin- 1-yl)-3- fluoropiperidin-1- yl)prop-2-en-1-one | 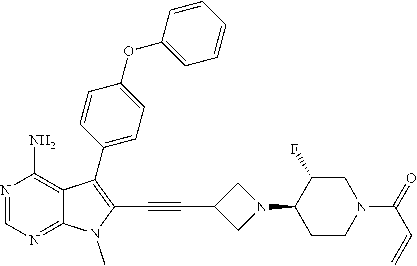 [SMILES: C=CC(=O)N1CC[C@@H](N2CC(C#Cc3c(-c4ccc(Oc5ccccc5)cc4)c4c(N)ncnc4n3C)C2)[C@H](F)C1] | ¹H NMR (400 MHz, DMSO-d₆) δ 8.19 (s, 1H), 7.53-7.51 (d, J = 8.1 Hz, 2H), 7.45-7.41 (t, J = 7.7 Hz, 2H), 7.20 − 7.13 (m, 5H), 6.82 − 6.72 (m, 1H), 6.22 (br, 1H), 6.11-6.07 (dd, J = 16.6, 2.3 Hz, 1H), 5.68-5.66 (d, J = 10.7 Hz, 1H), 4.71-4.59 (d, 1H), 4.37-3.87 (m, 2H), 3.72 (s, 3H), 3.61 − 3.51 (m, 2H), 3.49-3.46 (q, J = 7.5 Hz, 1H), 3.29-3.25(m, 1H), 3.15-3.06(q, J = 8.2, 7.2 Hz, 3H), 2.78 (t, J = 12.2 Hz, 0.5H), 2.45- 2.41 (m, 0.5H), 1.52 (s, 1H), 1.43 − 1.24 (m, 1H). MS: 551.25 |
| 1-((3S,4S)-4-(3-((4- amino-7-methyl-5- (4-phenoxyphenyl)- 7H-pyrrolo[2,3- d]pyrimidin-6- yl)ethynyl)azetidin- 1-yl)-3- fluoropiperidin-1- yl)prop-2-en-1-one | 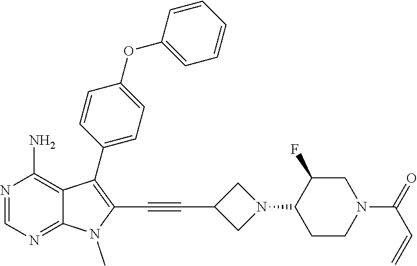 | ¹H NMR (400 MHz, DMSO-d₆) δ 8.19 (s, 1H), 7.53-7.51 (d, J = 8.1 Hz, 2H), 7.45-7.41 (t, J = 7.7 Hz, 2H), 7.20 − 7.12 (m, 5H), 6.82 − 6.72 (m, 1H), 6.22 (br, 1H), 6.11-6.07 (dd, J = 16.6, 2.3 Hz, 1H), 5.68-5.66 (d, J = 10.7 Hz, 1H), 4.71-4.59 (d, 1H), 4.37-3.87 (m, 2H), 3.72 (s, 3H), 3.61 − 3.51 (m, 2H), 3.49-3.46 (q, J = 7.5 Hz, 1H), 3.29-3.25(m, 1H), 3.15-3.06(q, J = 8.2, 7.2 Hz, 3H), 2.78 (t, J = 12.2 Hz, 0.5H), 2.45- 2.41 (m, 0.5H), 1.52 (s, 1H), 1.43 − 1.24 (m, 1H). MS: 551.25 |
| 1-(4-(3-((4-amino-7- methyl-5-(4- phenoxyphenyl)-7H- pyrrolo[2,3- d]pyrimidin-6- yl)ethynyl)azetidin- 1-yl)azepan-1- yl)prop-2-en-1-one | 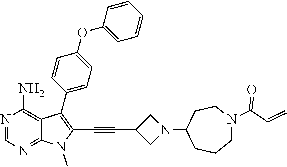 [SMILES: C=CC(=O)N1CCCC(N2CC(C#Cc3c(-c4ccc(Oc5ccccc5)cc4)c4c(N)ncnc4n3C)C2)CC1] | ¹H NMR (400 MHz, DMSO-d₆) δ 8.19 (s, 1H), 7.53 − 7.51 (m, 2H), 7.45 − 7.41 (m, 2H), 7.19 (t, J = 7.4 Hz, 1H), 7.17 − 7.12 (m, 4H), 6.74 − 6.67 (m, 1H), 6.12 (dr, 1H), 6.14 − 6.09 (m, 1H), 5.64 (dt, J = 10.3, 3.1 Hz, 1H), 3.72 (s, 3H), 3.54 − 3.51 (m, 4H), 3.47 − 3.34 (m, 3H), 2.96 (t, J = 6.8 Hz, 2H), 2.15 (s, 1H), 1.66 (s, 1H), 1.51 (d, J = 12.0 Hz, 1H), 1.52 − 1.49 (m, 2H), 1.36 − 1.14 (m, 2H). MS: 547.25 |
| 1-((2R)-4-(3-((4- amino-7-methyl-5- (4-phenoxyphenyl)- 7H-pyrrolo[2,3- d]pyrimidin-6- yl)ethynyl)azetidin- 1-yl)-2- methylpiperidin-1- yl)prop-2-en-1-one | 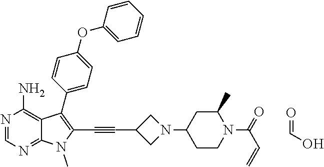 [SMILES: C=CC(=O)N1CCC(N2CC(C#Cc3c(-c4ccc(Oc5ccccc5)cc4)c4c(N)ncnc4n3C)C2)C[C@H]1C ; O=CO] | ¹H NMR (400 MHz, DMSO-d₆) δ 8.19 (s, 1H), 8.14 (s, 0.2H), 7.53 − 7.51 (m, 2H), 7.43 (t, J = 7.3 Hz, 2H), 7.18 (t, J = 7.3 Hz, 1H), 7.12 (d, J = 8.1 Hz, 4H), 6.73 (dd, J = 16.8, 10.7 Hz, 1H), 6.40-6.15(br, 1H) ,6.06 (d, J = 16.7 Hz, 1H), 5.63 (dd, J = 10.5, 2.9 Hz, 1H), 4.33 (m, 1H), 3.89 (m, 1H), 3.72 (d, J = 2.5 Hz, 3H), 3.58 (m, 1H), 3.51 (m, 1H), 3.40 (m, 1H), 3.07 (m, 2H), 2.96 (m, 1H), 2.42 (m, 1H), 1.55 (m, 1H), 1.48- 1.40 (m, 3H), 1.29-1.20 (m, 3H). MS: 547.35 |
| 1-[(2S)-4-(3-[2-[4- amino-7-methyl-5- (4-phenoxyphenyl)- 7H-pyrrolo[2,3- d]pyrimidin-6- yl]ethynyl]azetidin- 1-yl)-2- methylpiperidin-1- yl]prop-2-en-1-one | 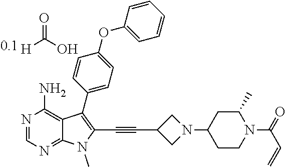 | ¹H NMR (400 MHz, DMSO-d₆) δ 8.19 (s, 1H), 7.53 − 7.51 (d, 2H), 7.45 − 7.41 (t, 2H), 7.20 − 7.16 (t, 1H), 7.13 − 7.10 (dd, 4H), 6.77 − 6.70 (dd, J = 16.7, 10.5 Hz, 1H), 6.20 (br, 1H), 6.08- 6.03 (dd, J = 16.7, 2.5 Hz, 1H), 5.64 − 5.61 (dd, J = 10.5, 2.5 Hz, 1H), 4.33 (br, 1H), 3.89 (br, 1H), 3.72 (s, 3H), 3.57 −3.55(t, J = 7.0 Hz, 1H), 3.50 −3.49 (t, J = 6.7 Hz, 1H), 3.42 −3.38 (q, J = 7.1 Hz, 1H), 3.06 −3.03 (t, J = 6.4 Hz, 1H), 2.96 − 2.92 (t, J = 6.5 Hz, 1H), 2.42 −2.40 (t, J = 3.7 Hz, 1H), 1.54 − 1.45 (m, 4H), 1.30 − 1.28 (d, |
| | | 3H). MS: 547.30 |
| 1-(4-(3-((4-amino-5- (4-phenoxyphenyl)- 7-(tetrahydrofuran- 3-yl)-7H- pyrrolo[2,3- d]pyrimidin-6- yl)ethynyl)azetidin- 1-yl)piperidin-1- yl)prop-2-en-1-one | 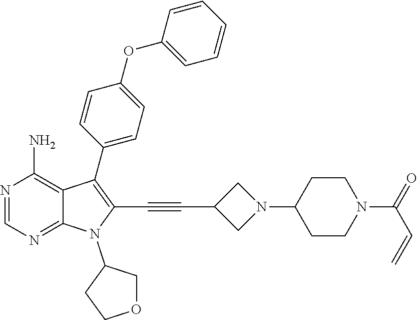 [SMILES: C=CC(=O)N1CCC(N2CC(C#Cc3c(-c4ccc(Oc5ccccc5)cc4)c4c(N)ncnc4n3C3CCOC3)C2)CC1] | ¹H NMR (400 MHz, DMSO-d₆) δ 8.19 (s, 1H), 7.56-7.50 (d, J = 8.6 Hz, 2H), 7.45 − 7.41 (t, 2H), 7.20 − 7.16 (t, 1H), 7.14-7.11(m, 4H), 6.78 (dd, J = 16.7, 10.5 Hz, 1H), 6.08 (dd, J = 16.6, 2.4 Hz, 1H), 5.63 (dd, J = 10.4, 2.5 Hz, 1H), 5.51 (p, J = 8.0 Hz, 1H), 4.22 (q, J = 8.0 Hz, 1H), 4.08 − 4.05 (m, 2H), 3.93 (q, J = 7.7 Hz, 2H), 3.78 (m, 1H), 3.52 (t, J = 7.0 Hz, 2H), 3.40 (m, J = 7.5 Hz, 1H), 3.16 (m, 1H), 2.99 (m, 3H), 2.62 (dt, J = 14.4, 6.7 Hz, 1H), 2.35 (m, 1H), 2.23 (m, 1H), 1.59 (m, 2H), 1.06 (m, 2H). MS: 589.30 |
| 1-((2R)-4-(3-((4- amino-7-methyl-5- (4-phenoxyphenyl)- 7H-pyrrolo[2,3- d]pyrimidin-6- yl)ethynyl)azetidin- 1-yl)-2- (hydroxymethyl) piperidin-1- yl)prop-2-en- 1-one | 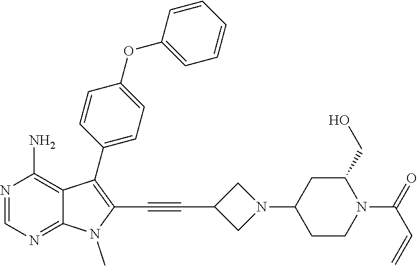 [SMILES: C=CC(=O)N1CCC(N2CC(C#Cc3c(-c4ccc(Oc5ccccc5)cc4)c4c(N)ncnc4n3C)C2)C[C@@H]1CO] | ¹H NMR (400 MHz, CDCl₃) δ 8.33 (s, 1H), 7.52 (d, J = 8.3 Hz, 2H), 7.43 (t, J = 7.8, 7.8 Hz, 2H), 7.21 (t, J = 7.4, 7.4 Hz, 1H), 7.13 (dd, J = 8.3, 6.2 Hz, 4H), 6.59 (dd, J = 16.9, 10.6 Hz, 1H), 6.28 (d, J = 16.8 Hz, 1H), 5.87 (br, 2H), 5.69 (dd, J = 10.6, 1.9 Hz, 1H), 4.72 (s, 1H), 3.89 (s, 4H), 3.74 (dt, J = 25.0, 7.1, 7.1 Hz, 4H), 3.54 − 3.43 (m, 1H), 3.23 (dt, J = 22.8, 7.0, 7.0 Hz, 2H), 2.61 (s, 1H), 1.94 (ddd, J = 14.7, 7.3, 3.8 Hz, 2H), 1.66 (m, 4H). MS: 563.35 |
| 1-(4-(3-((4-amino-7- methyl-5-(4- phenoxyphenyl)-7H- pyrrolo[2,3- d]pyrimidin-6- yl)ethynyl)pyrrolidin- 1-yl)piperidin-1- yl)prop-2-en-1-one formate | 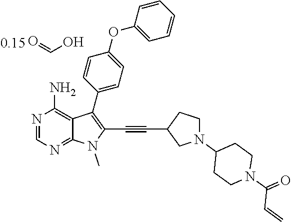 | ¹H NMR (400 MHz, DMSO-d₆) δ 8.17 (s, 1H), 7.51 − 7.44 (m, 2H), 7.41 (t, J = 7.9 Hz, 2H), 7.17 (t, J = 7.4 Hz, 1H), 7.16 − 7.10 (m, 4H), 6.77 (t, J = 13.8 Hz, 1H), 6.18 (s, 1H), 6.06 (dd, J = 16.7, 2.5 Hz, 1H), 5.64 (dd, J = 10.4, 2.5 Hz, 1H), 4.18 (d, J = 13.2 Hz, 1H), 3.91 (d, J = 13.3 Hz, 1H), 3.70 (s, 3H), 3.19 (q, J = 7.5 Hz, 1H), 3.10 (t, J = 12.4 Hz, 1H), 2.98 (t, J = 8.3 Hz, 1H), 2.81 (t, J = 12.3 Hz, 1H), 2.66 (s, 1H), 2.54 (s, 1H), 2.50 − 2.45 (m, 1H), 2.30 − 2.25 (m, 1H), 2.15 (dq, J = 16.7, 7.6 Hz, 1H), 1.80 (q, J = 5.8 Hz, 3H), 1.25 (s, 2H). MS: 547.35 |

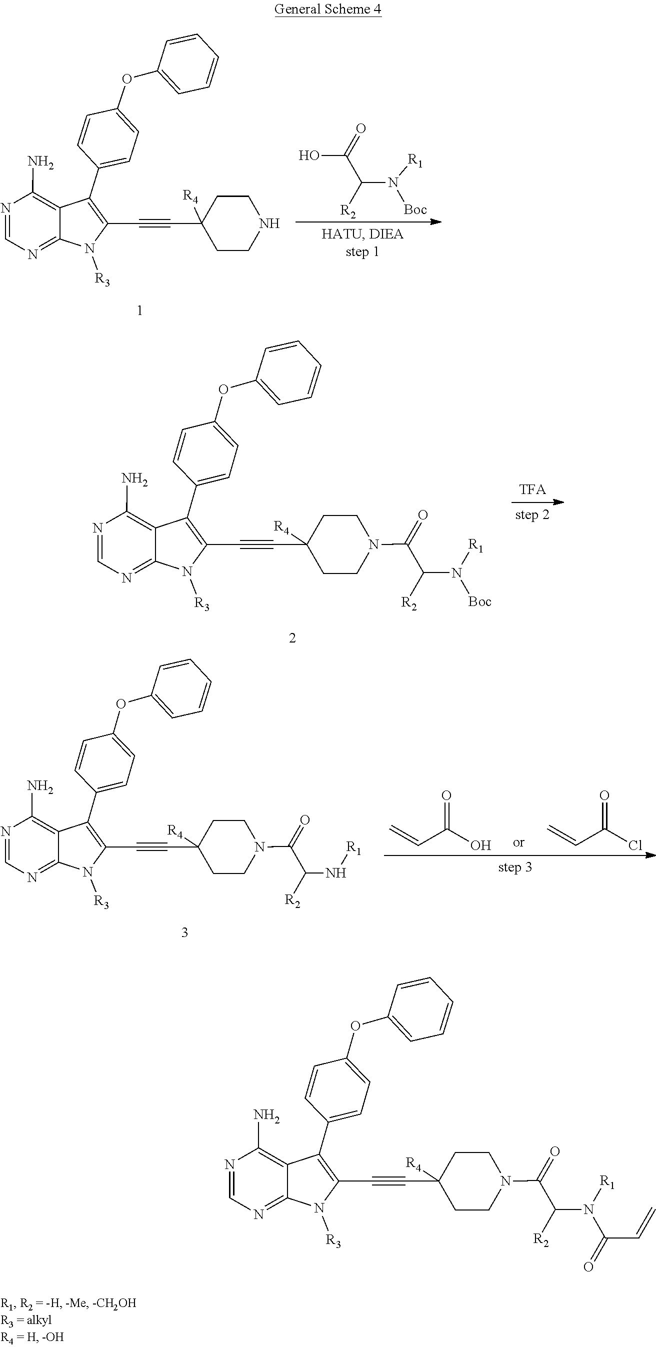

### Example 9

### N-(2-(4-((4-amino-7-isopropyl-5-(4-phenoxyphenyl)-7H-pyrrolo[2,3-d]pyrimidin-6-yl)ethynyl)piperidin-1-yl)-2-oxoethyl)acrylamide

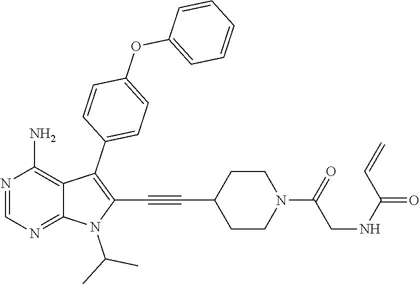 [SMILES: C=CC(=O)NCC(=O)N1CCC(C#Cc2c(-c3ccc(Oc4ccccc4)cc3)c3c(N)ncnc3n2C(C)C)CC1]

### 1-(4-((4-amino-7-isopropyl-5-(4-phenoxyphenyl)-7H-pyrrolo[2,3-d]pyrimidin-6-yl)ethynyl)piperidin-1-yl)-2-chloroethanone

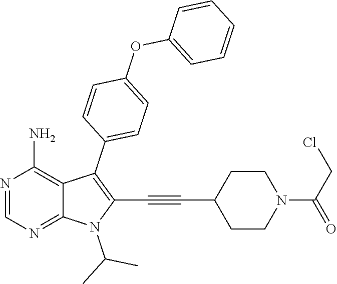 [SMILES: CC(C)n1c(C#CC2CCN(C(=O)CCl)CC2)c(-c2ccc(Oc3ccccc3)cc2)c2c(N)ncnc21]

### Step 1

A round bottomed flask was charged with 7-isopropyl-5-(4-phenoxyphenyl)-6-(piperidin-4-ylethynyl)-7H-pyrrolo[2,3-d]pyrimidin-4-amine (150 mg, 332 μmol), DCM (4 mL), TEA (100 mg, 996 μmol) and a stirbar. The solution was cooled to 0° C., then 2-chloroacetyl chloride (33.6 mg, 298 μmol) was added, and the mixture was stirred for 1 h at 0° C. The reaction mixture was quenched with MeOH (1 mL), and the solution was concentrated in vacuo. The resulting crude material was purified by Flash chromatography (eluting with acetonitrile/water=7/3). Concentration in vacuo resulted in 1-(4-((4-amino-7-isopropyl-5-(4-phenoxyphenyl)-7H-pyrrolo[2,3-d]pyrimidin-6-yl)ethynyl)piperidin-1-yl)-2-chloroethanone (90 mg, 51.4%) as a yellow amorphous solid.

### 2-amino-1-(4-((4-amino-7-isopropyl-5-(4-phenoxyphenyl)-7H-pyrrolo[2,3-d]pyrimidin-6-yl)ethynyl)piperidin-1-yl)ethanone

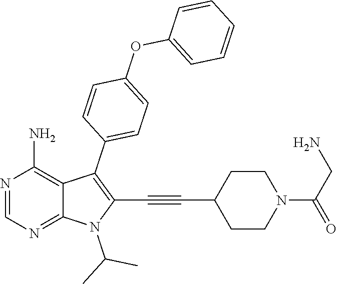 [SMILES: CC(C)n1c(C#CC2CCN(C(=O)CN)CC2)c(-c2ccc(Oc3ccccc3)cc2)c2c(N)ncnc21]

### Step 2

A round bottomed flask was charged with 1-(4-((4-amino-7-isopropyl-5-(4-phenoxyphenyl)-7H-pyrrolo[2,3-d]pyrimidin-6-yl)ethynyl)piperidin-1-yl)-2-chloroethanone (80 mg, 151 μmol), NH₃/MeOH (2 mL) and a stirbar. The mixture was stirred overnight at 40° C. The solution was concentrated in vacuo and the resulting crude material was purified by Flash chromatography (eluting with acetonitrile/water=31/69). Concentration in vacuo resulted in 2-amino-1-(4-((4-amino-7-isopropyl-5-(4-phenoxyphenyl)-7H-pyrrolo[2,3-d]pyrimidin-6-yl)ethynyl)piperidin-1-yl)ethanone (60 mg, 78.1%) as a tan solid.

### N-(2-(4-((4-amino-7-isopropyl-5-(4-phenoxyphenyl)-7H-pyrrolo[2,3-d]pyrimidin-6-yl)ethynyl)piperidin-1-yl)-2-oxoethyl)acrylamide

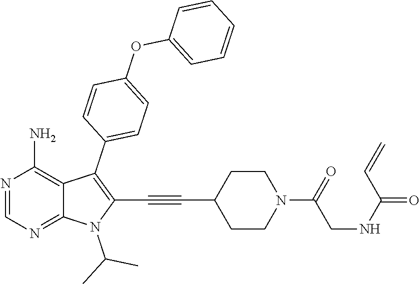 [SMILES: C=CC(=O)NCC(=O)N1CCC(C#Cc2c(-c3ccc(Oc4ccccc4)cc3)c3c(N)ncnc3n2C(C)C)CC1]

### Step 3

A round bottomed flask was charged with 2-amino-1-(4-((4-amino-7-isopropyl-5-(4-phenoxyphenyl)-7H-pyrrolo[2,3-d]pyrimidin-6-yl)ethynyl)piperidin-1-yl)ethanone (50 mg, 98 mol), DCM (2 mL), TEA (30 mg, 294 μmol) and a stirbar. The solution was cooled to 0° C., then acryloyl chloride (8 mg, 88 μmol) was added, and the mixture was stirred for 1 h at 0° C. The reaction mixture was quenched with MeOH (1 mL), and the solution was concentrated in vacuo. The resulting crude material was purified by HPLC. Column: XBridge Prep C18 OBD Column, 5 um, 19*150 mm; Mobile Phase A:Water (10 MMOL/L NH₄HCO₃), Mobile Phase B: MeOH-HPLC; Flow rate: 20 mL/min; Gradient: 67% B to 67% B in 8 min, then from 67% B to 0% B, From 8 to 0 min; 254/220 nm; RT1:7.15. Lyophilization yielded N-(2-(4-((4-amino-7-isopropyl-5-(4-phenoxyphenyl)-7H-pyrrolo[2,3-d]pyrimidin-6-yl)ethynyl)piperidin-1-yl)-2-oxoethyl)acrylamide (25.3 mg, 45.8%) as a off-white amorphous solid.

### Example 10

### N-(2-(4-((4-amino-7-methyl-5-(4-phenoxyphenyl)-7H-pyrrolo[2,3-d]pyrimidin-6-yl)ethynyl)piperidin-1-yl)-2-oxoethyl)-N-methylacrylamide

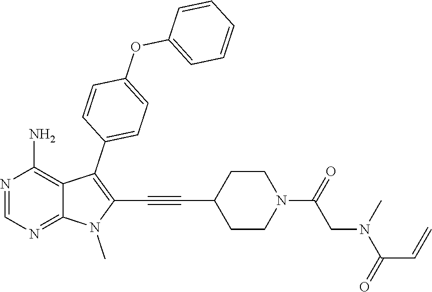 [SMILES: C=CC(=O)N(C)CC(=O)N1CCC(C#Cc2c(-c3ccc(Oc4ccccc4)cc3)c3c(N)ncnc3n2C)CC1]

### tert-butyl-2-(4-((4-amino-7-methyl-5-(4-phenoxyphenyl)-7H-pyrrolo[2,3-d]pyrimidin-6-yl)ethynyl)piperidin-1-yl)-2-oxoethyl(methyl)carbamate

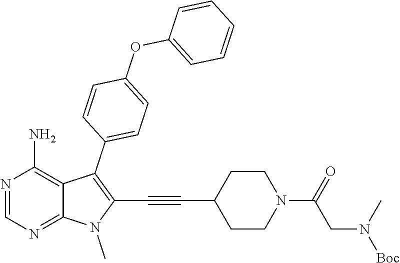 [SMILES: CN(CC(=O)N1CCC(C#Cc2c(-c3ccc(Oc4ccccc4)cc3)c3c(N)ncnc3n2C)CC1)C(=O)OC(C)(C)C]

### Step 1

2-{[(tert-butoxy)carbonyl](methyl)amino}acetic acid (44.6 mg, 0.2357 mmol) was added to HATU (134 mg, 0.3541 mmol) and TEA (71.5 mg, 0.7083 mmol) in DMF (2 mL) at 25° C. and the resulting solution was stirred at RT for 10 minutes. 7-methyl-5-(4-phenoxyphenyl)-6-[2-(piperidin-4-yl)ethynyl]-7H-pyrrolo[2,3-d]pyrimidin-4-amine (100 mg, 0.2361 mmol) was added to the mixture solution and the reaction mixture was stirred at RT for 30 minutes. The crude product was purified by flash C18-flash chromatography, elution gradient 0 to 100% MeCN in water. Pure fractions were evaporated to dryness to afford tert-butyl N-[2-(4-{2-[4-amino-7-methyl-5-(4-phenoxyphenyl)-7H-pyrrolo[2,3-d]pyrimidin-6-yl]ethynyl}piperidin-1-yl)-2-oxoethyl]-N-methylcarbamate (100 mg, 71.4%) as a yellow solid.

### 1-(4-((4-amino-7-methyl-5-(4-phenoxyphenyl)-7H-pyrrolo[2,3-d]pyrimidin-6-yl)ethynyl)piperidin-1-yl)-2-(methylamino)ethanone

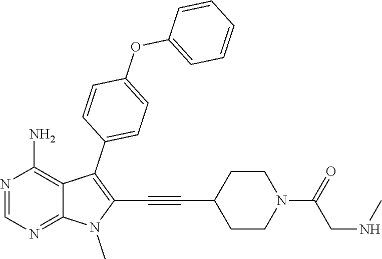 [SMILES: CNCC(=O)N1CCC(C#Cc2c(-c3ccc(Oc4ccccc4)cc3)c3c(N)ncnc3n2C)CC1]

### Step 2

TFA (0.5 mL, 0.1681 mmol) was added to tert-butyl N-[2-(4-{2-[4-amino-7-methyl-5-(4-phenoxyphenyl)-7H-pyrrolo[2,3-d]pyrimidin-6-yl]ethynyl}piperidin-1-yl)-2-oxoethyl]-N-methylcarbamate (100 mg, 0.1681 mmol) in DCM (2 mL) at 25° C. The resulting solution was stirred at RT for 30 minutes. The solvent was removed under reduced pressure. The product was used in the next step directly without further purification.

### N-(2-(4-((4-amino-7-methyl-5-(4-phenoxyphenyl)-7H-pyrrolo[2,3-d]pyrimidin-6-yl)ethynyl)piperidin-1-yl)-2-oxoethyl)-N-methylacrylamide

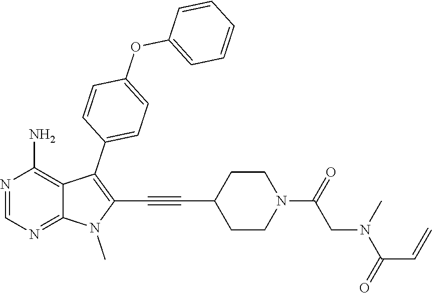 [SMILES: C=CC(=O)N(C)CC(=O)N1CCC(C#Cc2c(-c3ccc(Oc4ccccc4)cc3)c3c(N)ncnc3n2C)CC1]

### Step 3

prop-2-enoic acid (9.61 mg, 0.1334 mmol) was added to HATU (46.0 mg, 0.1213 mmol) and TEA (12.2 mg, 0.1213 mmol) in DMF (2 mL) at 25° C. and the resulting solution was stirred at RT for 10 minutes. 1-(4-{2-[4-amino-7-methyl-5-(4-phenoxyphenyl)-7H-pyrrolo[2,3-d]pyrimidin-6-yl]ethynyl}piperidin-1-yl)-2-(methylamino)ethan-1-one (60 mg, 0.1213 mmol) was added to the mixture solution and the reaction mixture was stirred at RT for 30 minutes. The crude product was purified by preparative HPLC (Column: XBridge Shield RP18 OBD Column, 5 um, 19*150 mm; Mobile Phase A:Water (10 MMOL/L NH4HCO3), Mobile Phase B: ACN; Flow rate: 20 mL/min; Gradient: 10% B to 60% B in 7 min; 220 nm; Rt: Array min). Fractions containing the desired compound were evaporated to dryness to afford N-[2-(4-{2-[4-amino-7-methyl-5-(4-phenoxyphenyl)-7H-pyrrolo[2,3-d]pyrimidin-6-yl]ethynyl}piperidin-1-yl)-2-oxoethyl]-N-methylprop-2-enamide (11.8 mg, 17.70) as a off-white amorphous solid.

Compounds prepared by substantially the same methods, and their characterization data, are listed in Table 8, below.

TABLE 8
Selected Compounds

| | | Proton NMR & |
|---|---|---|
| Compound | Structure | Mass Spec. (obs.) |
| N-(2-(4-((4-amino- 7-isopropyl-5-(4- phenoxyphenyl)- 7Hpyrrolo [2,3-d]pyrimidin-6- yl) ethynyl)piperidin-1- yl)-2-oxoethyl) acrylamide | 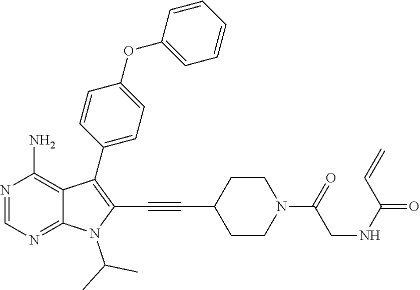 [SMILES: C=CC(=O)NCC(=O)N1CCC(C#Cc2c(-c3ccc(Oc4ccccc4)cc3)c3c(N)ncnc3n2C(C)C)CC1] | ¹H NMR (300 MHz, CDCl₃) δ 8.32 (s, 1H), 7.55 − 7.46 (m, 2H), 7.42 (dd, J = 8.5, 7.3 Hz, 2H), 7.19 (t, J = 7.4, 7.4 Hz, 1H), 7.16 − 7.05 (m, 4H), 6.76 (s, 1H), 6.34 (dd, J = 17.0, 1.8 Hz, 1H), 6.22 (dd, J = 17.0, 9.9 Hz, 1H), 5.71 (dd, J = 9.9, 1.8 Hz, 1H), 5.41 (s, 2H), 5.23 (p, J = 6.8, 6.8, 6.7, 6.7 Hz, 1H), 4.15 (t, J = 4.1, 4.1 Hz, 2H), 3.82 − 3.69 (m, 1H), 3.53 (dd, J = 23.2, 11.0 Hz, 2H), 3.33 (dd, J = 11.4, 6.3 Hz, 1H), 3.06 − 2.97 (m, 1H), 1.88 (d, J = 13.2 Hz, 2H), 1.75-1.70 (m, 8H). MS: 563.3 |
| N-(2-(4-((4-amino- 7-methyl-5-(4- phenoxyphenyl)-7H- pyrrolo[2,3- d]pyrimidin-6- yl)ethynyl)piperidin- 1-yl)-2-oxoethyl)-N- methylacrylamide | 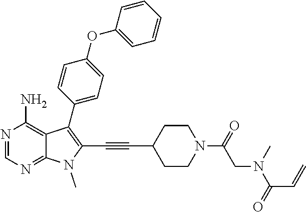 [SMILES: C=CC(=O)N(C)CC(=O)N1CCC(C#Cc2c(-c3ccc(Oc4ccccc4)cc3)c3c(N)ncnc3n2C)CC1] | ¹H NMR (400 MHz, DMSO-d₆) δ 8.19 − 8.16 (m, 1H), 7.52 (t, J = 7.3 Hz, 2H), 7.44 (q, J = 7.2 Hz, 2H), 7.18 − 7.11 (m, 5H), 6.23 (br, 2H), 5.28 − 4.84 (m, 1H), 4.30 − 4.14 (m, 2H), 3.64 − 3.50(m, 6H), 3.27 (s, 1H), 3.09 − 2.91 (m, 3H), 2.83 − 2.73 (m, 1H), 2.35 (q, J = 11.6, 9.6 Hz, 2H), 1.80 (s, 2H), 1.53 (s, 2H). MS: 549.55 |
| N-(2-(4-((4-amino- 7-methyl-5-(4- phenoxyphenyl)-7H- pyrrolo[2,3- d]pyrimidin-6- yl)ethynyl)-4- hydroxypiperidin-1- yl)-2- oxoethyl)acrylamide | 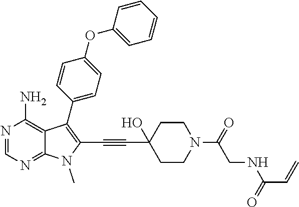 [SMILES: C=CC(=O)NCC(=O)N1CCC(O)(C#Cc2c(-c3ccc(Oc4ccccc4)cc3)c3c(N)ncnc3n2C)CC1] | ¹H NMR (400 MHz, DMSO-d₆) δ 8.22-8.20 (d, J = 3.9 Hz, 2H), 7.54 − 7.52 (m, 2H), 7.45 − 7.41 (m, 2H), 7.19-7.15(m, 1H),7.14 − 7.10 (m, 4H), 6.42-6.36 (dd, J = 17.1, 10.3 Hz, 1H), 6.12-6.07 (dd, J = 17.1, 2.2 Hz, 1H), 5.90 (s, 1H), 5.62-5.59 (dd, J = 10.2, 2.2 Hz, 1H), 4.07 − 4.03 (m, 2H), 3.83 (d, J = 13.1 Hz, 1H), 3.75 (s, 3H), 3.62 (s, 1H), 3.25(s,1H),3.16 (s, 1H), 1.88 −1.82(m, 2H), 1.70 (s, 1H), 1.59 (d, J = 10.1 Hz, 1H). MS: 551.30 |
| (S)-N-(1-(4-((4- amino-7-methyl-5- (4-phenoxyphenyl)- 7H-pyrrolo[2,3- d]pyrimidin-6- yl)ethynyl)piperidin- 1-yl)-1-oxopropan- 2-yl)acrylamide | 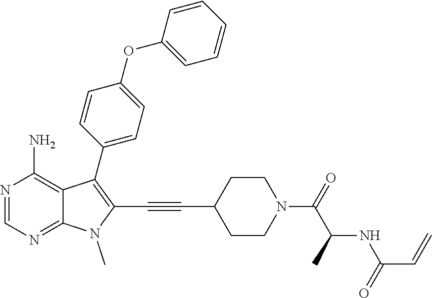 [SMILES: C=CC(=O)N[C@@H](C)C(=O)N1CCC(C#Cc2c(-c3ccc(Oc4ccccc4)cc3)c3c(N)ncnc3n2C)CC1] | ¹H NMR (400 MHz, Methanol-d₄) δ 8.20 (s, 1H), 7.56 (d, J = 6.8 Hz, 2H), 7.42 (s, 2H), 7.18 (s, 1H), 7.13 (dd, J = 16.3, 8.3 Hz, 4H), 6.32 − 6.23 (m, 2H), 5.69 (t, J = 8.5 Hz, 1H), 4.90 (t, J = 8.5 Hz, 1H), 3.83 (d, J = 3.7 Hz, 3H), 3.72 (s, 1H), 3.62 (s, 1H), 3.52 (s, 1H), 3.10 (d, J = 23.9 Hz, 2H), 1.91 (d, J = 34.6 Hz, 2H), 1.35 (t, J = 7.4 Hz, 3H). MS: 549.30 |
| (R)-N-(1-(4-((4- amino-7-methyl-5- (4-phenoxyphenyl)- 7H-pyrrolo[2,3- d]pyrimidin-6- yl)ethynyl)piperidin- 1-yl)-1-oxopropan- 2-yl)acrylamide | 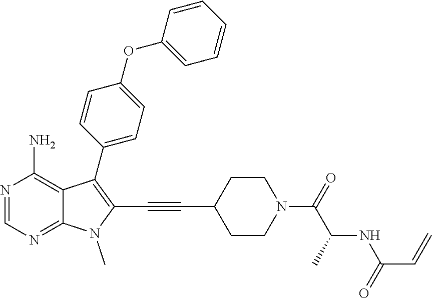 [SMILES: C=CC(=O)N[C@H](C)C(=O)N1CCC(C#Cc2c(-c3ccc(Oc4ccccc4)cc3)c3c(N)ncnc3n2C)CC1] | ¹H NMR (400 MHz, Methanol-d₄) δ 8.19 (s, 1H), 7.54 (m, 2H), 7.44 − 7.39 (m, 2H), 7.15 (td, J = 15.6, 6.4 Hz, 5H), 6.32 − 6.21 (m, 2H), 5.70 − 5.66 (m, 1H), 4.95 (s, 1H), 3.83 (d, J = 3.9 Hz, 4H), 3.65 (d, J = 5.6 Hz, 1H), 3.53 − 3.50 (m, 1H), 3.11-3.05 (m, 1H), 3.05 (s, 1H), 1.99-1.89 (m, 2H), 1.75-1.66 (m, 2H), 1.32 (t, J = 7.5 Hz, 3H). MS: 549.30 |
| N-((R)-1-(4-((4- amino-5-(4- phenoxyphenyl)-7- ((S)-tetrahydrofuran- 3-yl)-7H- pyrrolo[2,3- d]pyrimidin-6- yl)ethynyl)piperidin- 1-yl)-1-oxopropan- 2-yl)acrylamide | 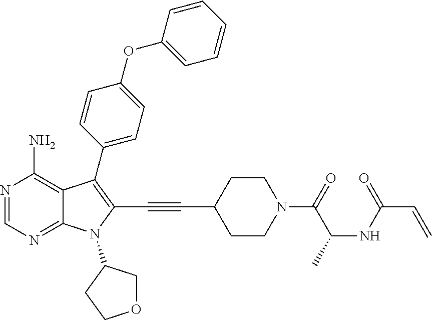 | ¹H NMR (400 MHz, DMSO-d₆) δ 8.40 (s, 1H), 8.18 (s, 1H), 7.51 (d, J = 8.0 Hz, 2H), 7.43 (t, J = 7.8 Hz, 2H), 7.19 − 7.10 (m, 5H), 6.31 (dd, J = 17.1, 10.2 Hz, 1H), 6.09 (d, J = 16.8 Hz, 1H), 5.60 − 5.50 (m, 2H), 4.82 (d, J = 8.8 Hz, 1H), 4.20 − 4.17 (m, 1H), 4.08 (q, J = 7.7, 6.4 Hz, 2H), 3.93 − 3.91 (m, 1H), 3.85 − 3.60 (m, 1H), 3.51 (s, 1H), 3.43 (s, 1H), 3.15 (s, 1H), 3.04 (s, 1H), 2.62 (s, 1H), 2.49 (s, 1H), 1.80 (s, 2H), 1.54 (s, 2H), 1.17 (d, J = 7.0 Hz, 3H). MS: 605.25 |

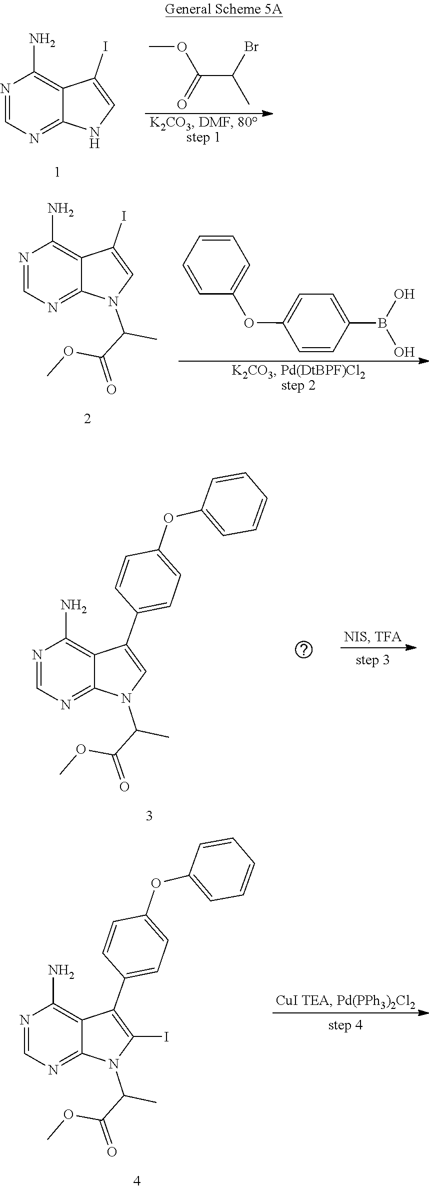

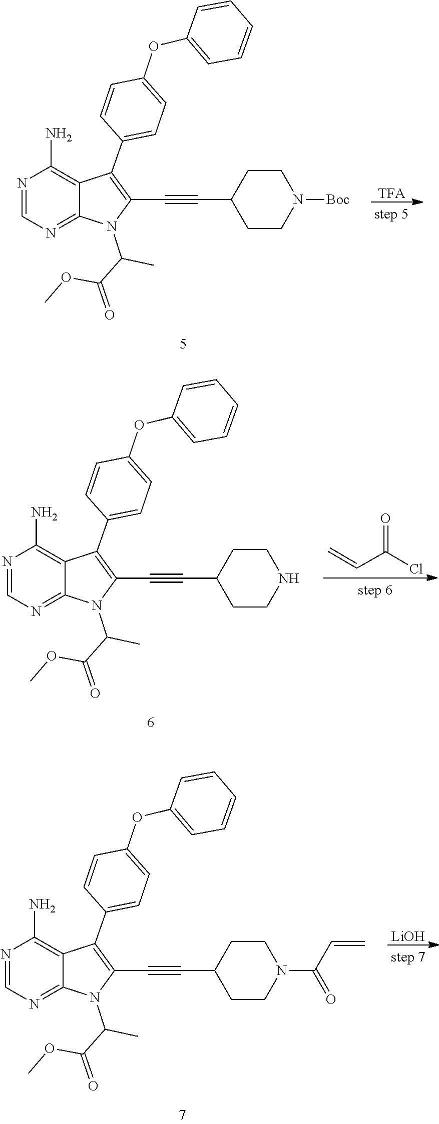

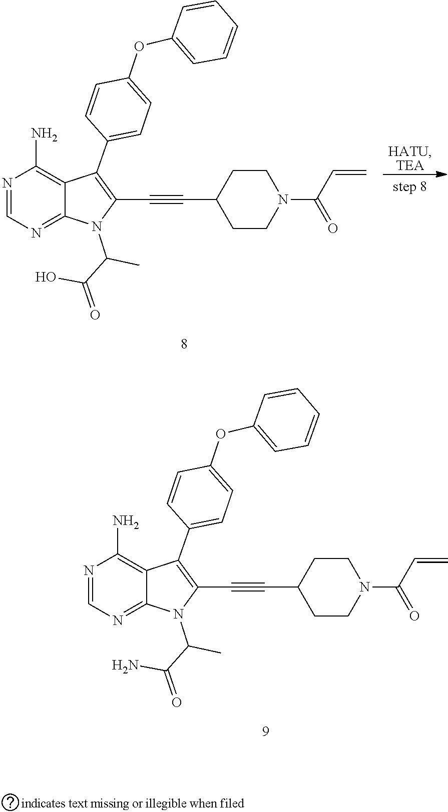

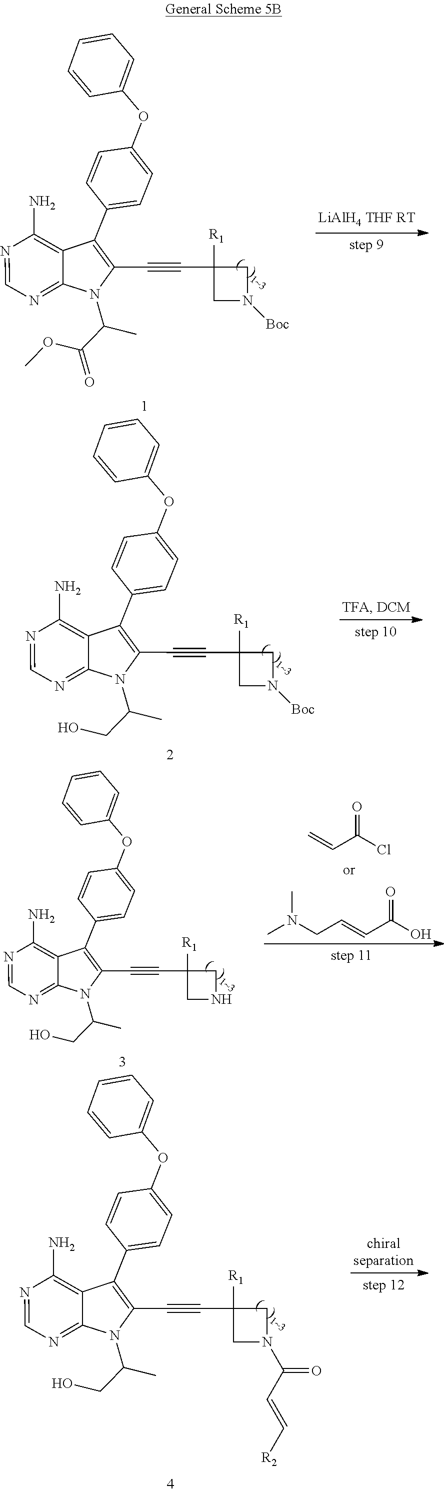

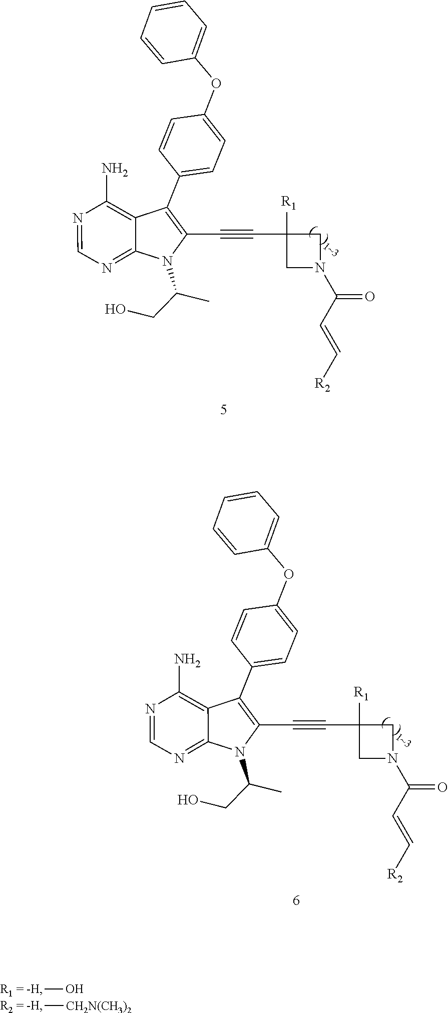

### Example 11

### 2-(6-((1-acryloylpiperidin-4-yl)ethynyl)-4-amino-5-(4-phenoxyphenyl)-7H-pyrrolo[2,3-d]pyrimidin-7-yl)propanamide

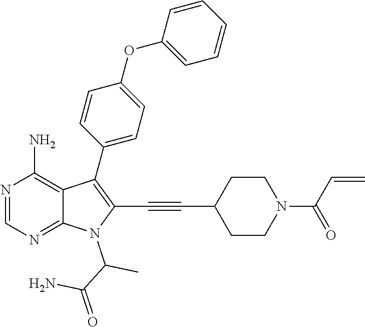 [SMILES: C=CC(=O)N1CCC(C#Cc2c(-c3ccc(Oc4ccccc4)cc3)c3c(N)ncnc3n2C(C)C(N)=O)CC1]

### methyl 2-(6-((1-acryloylpiperidin-4-yl)ethynyl)-4-amino-5-(4-phenoxyphenyl)-7H-pyrrolo[2,3-d]pyrimidin-7-yl)propanoate

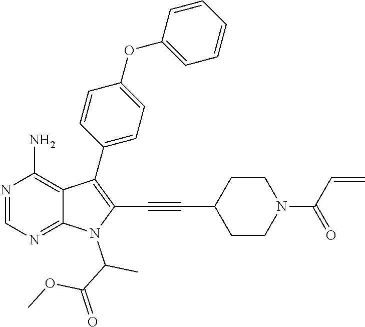 [SMILES: C=CC(=O)N1CCC(C#Cc2c(-c3ccc(Oc4ccccc4)cc3)c3c(N)ncnc3n2C(C)C(=O)OC)CC1]

### 2-(6-((1-acryloylpiperidin-4-yl)ethynyl)-4-amino-5-(4-phenoxyphenyl)-7H-pyrrolo[2,3-d]pyrimidin-7-yl)propanoic acid, and

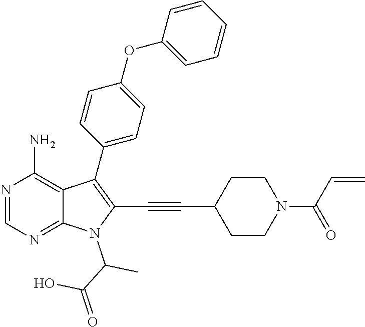 [SMILES: C=CC(=O)N1CCC(C#Cc2c(-c3ccc(Oc4ccccc4)cc3)c3c(N)ncnc3n2C(C)C(=O)O)CC1]

### methyl 2-(4-amino-5-(4-phenoxyphenyl)-6-(piperidin-4-ylethynyl)-7H-pyrrolo[2,3-d]pyrimidin-7-yl)propanoate

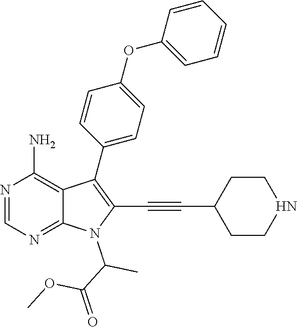 [SMILES: COC(=O)C(C)n1c(C#CC2CCNCC2)c(-c2ccc(Oc3ccccc3)cc2)c2c(N)ncnc21]

### methyl 2-(4-amino-5-iodo-7H-pyrrolo[2,3-d]pyrimidin-7-yl)propanoate

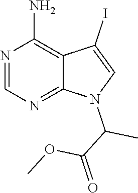 [SMILES: COC(=O)C(C)n1cc(I)c2c(N)ncnc21]

### Step 1

methyl 2-bromopropanoate (1.68 g, 10.1 mmol) was added to 5-iodo-7H-pyrrolo[2,3-d]pyrimidin-4 amine (2.2 g, 8.46 mmol) and K₂CO₃ (3.49 g, 25.3 mmol) in DMF (10 mL) at 25° C. The resulting solution was stirred at 80° C. for 2 hours. The reaction mixture was diluted with H₂O (100 mL), and the aqueous phase was extracted with ethyl acetate (400 mL) three times. The combined organic layers were washed with saturated brines, dried over sodium sulfate, filtered, and concentrated in vacuo. The crude product was purified by flash C18-flash chromatography, elution gradient 0 to 100% MeCN in water. Pure fractions were evaporated to dryness to afford methyl 2-(4-amino-5-iodo-7H-pyrrolo[2,3-d]pyrimidin-7-yl)propanoate (2.40 g, 82.1%) as a tan amorphous solid.

### methyl 2-(4-amino-5-(4-phenoxyphenyl)-7H-pyrrolo[2,3-d]pyrimidin-7-yl)propanoate

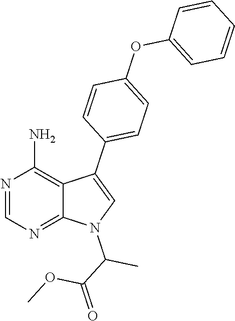 [SMILES: COC(=O)C(C)n1cc(-c2ccc(Oc3ccccc3)cc2)c2c(N)ncnc21]

### Step 2

Pd(DtBPF)Cl₂ (374 mg, 576 μmol) was added to methyl 2-(4-amino-5-iodo-7H-pyrrolo[2,3-d]pyrimidin-7-yl)propanoate (1 g, 2.88 mmol), (4-phenoxyphenyl)boronic acid (1.23 g, 5.76 mmol) and K₂CO₃ (1.19 g, 8.63 mmol) in water (2 mL) and 1,4-dioxane (16 mL) at 25° C. under nitrogen. The resulting solution was stirred at 100° C. for 2 hours. The reaction mixture was diluted with H₂O (20 mL), and the aqueous phase was extracted with ethyl acetate (200 mL) three times. The combined organic layers were washed with saturated brines, dried over sodium sulfate, filtered, and concentrated in vacuo. The crude product was purified by flash C18-flash chromatography, elution gradient 0 to 100% MeCN in water. Pure fractions were evaporated to dryness to afford methyl 2-(4-amino-5-(4-phenoxyphenyl)-7H-pyrrolo[2,3-d]pyrimidin-7-yl)propanoate (910 mg, 80.9%) as a tan amorphous solid.

### methyl 2-(4-amino-6-iodo-5-(4-phenoxyphenyl)-7H-pyrrolo[2,3-d]pyrimidin-7-yl)propanoate

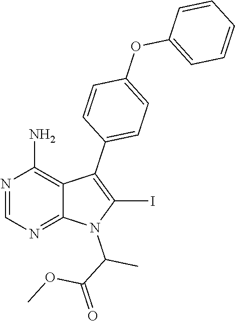

### Step 3

TFA (291 mg, 2.56 mmol) was added to methyl 2-(4-amino-5-(4-phenoxyphenyl)-7H-pyrrolo[2,3-d]pyrimidin-7-yl)propanoate (500 mg, 1.28 mmol) and NIS (315 mg, 1.40 mmol) in DCM (10 mL) at the room temperature. The resulting solution was stirred at RT for 5 hours. The solvent was removed under reduced pressure, The crude product was purified by flash C18-flash chromatography, elution gradient 0 to 100% MeCN in water. Pure fractions were evaporated to dryness to afford methyl 2-(4-amino-6-iodo-5-(4-phenoxyphenyl)-7H-pyrrolo[2,3-d]pyrimidin-7-yl)propanoate (400 mg, 60.7%) as a off-white amorphous solid.

### tert-butyl 4-((4-amino-7-(1-methoxy-1-oxopropan-2-yl)-5-(4-phenoxyphenyl)-7H-pyrrolo[2,3-d]pyrimidin-6-yl)ethynyl)piperidine-1-carboxylate

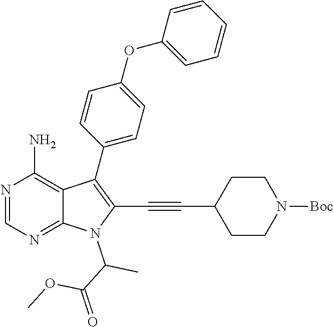 [SMILES: COC(=O)C(C)n1c(C#CC2CCN(C(=O)OC(C)(C)C)CC2)c(-c2ccc(Oc3ccccc3)cc2)c2c(N)ncnc21]

### Step 4

TEA (382 mg, 3.79 mmol) was added to methyl 2-(4-amino-6-iodo-5-(4-phenoxyphenyl)-7H-pyrrolo[2,3-d]pyrimidin-7-yl)propanoate (390 mg, 758 μmol), tert-butyl 4-ethynylpiperidine-1-carboxylate (316 mg, 1.51 mmol), CuI (28.6 mg, 151 μmol) and Pd(PPh₃)₂Cl₂ (105 mg, 151 μmol) in THF (10 mL) at RT. The resulting solution was stirred at 60° C. for 2 hours. The solvent was removed under reduced pressure, The crude product was purified by flash C18-flash chromatography, elution gradient 0 to 100% MeCN in water. Pure fractions were evaporated to dryness to afford tert-butyl 4-((4-amino-7-(1-methoxy-1-oxopropan-2-yl)-5-(4-phenoxyphenyl)-7H-pyrrolo[2,3-d]pyrimidin-6-yl)ethynyl)piperidine-1-carboxylate (450 mg, 60.9%) as a off-white amorphous solid.

### methyl 2-(4-amino-5-(4-phenoxyphenyl)-6-(piperidin-4-ylethynyl)-7H-pyrrolo[2,3-d]pyrimidin-7-yl)propanoate

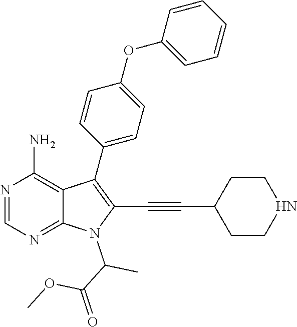 [SMILES: COC(=O)C(C)n1c(C#CC2CCNCC2)c(-c2ccc(Oc3ccccc3)cc2)c2c(N)ncnc21]

### Step 5

TFA (1 mL, 100 μmol) was added to tert-butyl 4-((4-amino-7-(1-methoxy-1-oxopropan-2-yl)-5-(4-phenoxyphenyl)-7H-pyrrolo[2,3-d]pyrimidin-6-yl)ethynyl)piperidine-1-carboxylate (60 mg, 100 μmol) in DCM (4 mL) at RT. The resulting solution was stirred at RT for 2 hours. The solvent was removed under reduced pressure, The crude product was purified by preparative HPLC (Column: XBridge Prep C18 OBD Column 19×150 mm 5 um; Mobile Phase A:Water (10 MMOL/L NH₄HCO₃), Mobile Phase B: ACN; Flow rate: 20 mL/min; Gradient: 25% B to 55% B in 7 min; 254/220 nm; Rt: 6.5 min). Fractions containing the desired compound were evaporated to dryness to afford methyl 2-(4-amino-5-(4-phenoxyphenyl)-6-(piperidin-4-ylethynyl)-7H-pyrrolo[2,3-d]pyrimidin-7-yl)propanoate (12.4 mg, 25%) as a off-white amorphous solid.

### methyl 2-(6-((1-acryloylpiperidin-4-yl)ethynyl)-4-amino-5-(4-phenoxyphenyl)-7H-pyrrolo[2,3-d]pyrimidin-7-yl)propanoate

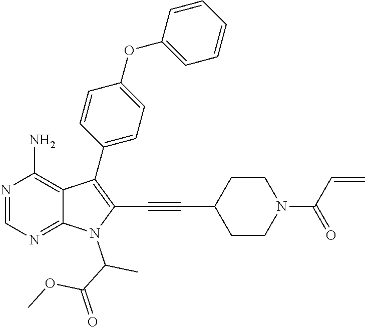 [SMILES: C=CC(=O)N1CCC(C#Cc2c(-c3ccc(Oc4ccccc4)cc3)c3c(N)ncnc3n2C(C)C(=O)OC)CC1]

### Step 6

acryloyl chloride (7.30 mg, 80.7 μmol) was dropwise added to methyl 2-(4-amino-5-(4-phenoxyphenyl)-6-(piperidin-4-ylethynyl)-7H-pyrrolo[2,3-d]pyrimidin-7-yl)propanoate (40 mg, 80.7 μmol) and TEA (24.4 mg, 242 μmol) in DCM (2 mL) at 0° C. The resulting solution was stirred at RT for 1 hour. The crude product was purified by preparative HPLC (Column: XBridge Prep C18 OBD Column 19×150 mm 5 um; Mobile Phase A:Water (10 MMOL/L NH₄HCO₃), Mobile Phase B: ACN; Flow rate: 20 mL/min; Gradient: 35% B to 60% B in 7 min; 254&220 nm; Rt: 6.5 min). Fractions containing the desired compound were evaporated to dryness to afford methyl 2-(6-((1-acryloylpiperidin-4-yl)ethynyl)-4-amino-5-(4-phenoxyphenyl)-7H-pyrrolo[2,3-d]pyrimidin-7-yl)propanoate (11.0 mg, 25%) as a white amorphous solid.

### 2-(6-((1-acryloylpiperidin-4-yl)ethynyl)-4-amino-5-(4-phenoxyphenyl)-7H-pyrrolo[2,3-d]pyrimidin-7-yl)propanoic acid

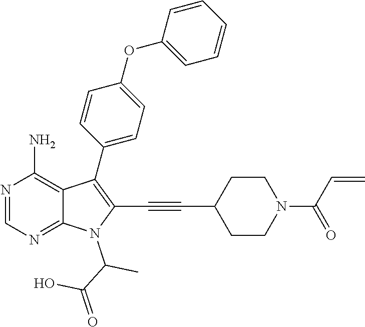 [SMILES: C=CC(=O)N1CCC(C#Cc2c(-c3ccc(Oc4ccccc4)cc3)c3c(N)ncnc3n2C(C)C(=O)O)CC1]

### Step 7

LiOH (1 mL, 109 μmol) was added to methyl 2-(6-((1-acryloylpiperidin-4-yl)ethynyl)-4-amino-5-(4-phenoxyphenyl)-7H-pyrrolo[2,3-d]pyrimidin-7-yl)propanoate (30 mg, 54.5 μmol) in THE (1 mL) and MeOH (1 mL) at RT. The resulting solution was stirred at R^{†} for 2 hours. and acidified to pH 5 with 1N HCl, whereupon ethyl acetate (50 mL) was added. The combined organic layers were washed with brine (10 mL), dried over magnesium sulfate, and concentrated. The crude product was purified by preparative HPLC (Column: XSelect CSH Prep C18 OBD Column, 5 um, 19*150 mm; Mobile Phase A:Water (0.1% FA), Mobile Phase B: ACN; Flow rate: 20 mL/min; Gradient: 20% B to 40% B in 10 min; 254/220 nm; Rt: 10.2 min). Fractions containing the desired compound were evaporated to dryness to afford 2-(6-((1-acryloylpiperidin-4-yl)ethynyl)-4-amino-5-(4-phenoxyphenyl)-7H-pyrrolo[2,3-d]pyrimidin-7-yl)propanoic acid (9.00 mg, 30.6%) as a off-white amorphous solid.

### 2-(6-((1-acryloylpiperidin-4-yl)ethynyl)-4-amino-5-(4-phenoxyphenyl)-7H-pyrrolo[2,3-d]pyrimidin-7-yl)propanamide

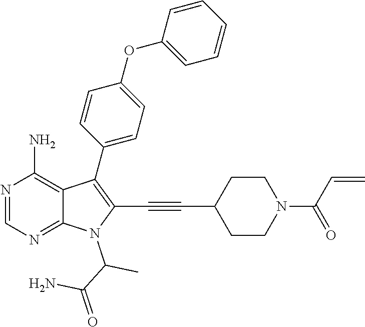 [SMILES: C=CC(=O)N1CCC(C#Cc2c(-c3ccc(Oc4ccccc4)cc3)c3c(N)ncnc3n2C(C)C(N)=O)CC1]

### Step 8

HATU (42.1 mg, 111 μmol) was added to 2-(6-((1-acryloylpiperidin-4-yl)ethynyl)-4-amino-5-(4-phenoxyphenyl)-7H-pyrrolo[2,3-d]pyrimidin-7-yl)propanoic acid (40 mg, 74.6 μmol), TEA (22.5 mg, 223 μmol) and NH4Cl (12.0 mg, 223 μmol) in DMF (1 mL) at RT. The resulting solution was stirred at RT for 1 hour. The crude product was purified by preparative HPLC (Column: XBridge Prep C18 OBD Column 19×150 mm 5 um; Mobile Phase A:Water (10 MMOL/L NH₄HCO₃), Mobile Phase B: ACN; Flow rate: 20 mL/min; Gradient: 27% B to 43% B in 7 min; 254&220 nm; Rt: 5.8 min). Fractions containing the desired compound were evaporated to dryness to afford 2-(6-((1-acryloylpiperidin-4-yl)ethynyl)-4-amino-5-(4-phenoxyphenyl)-7H-pyrrolo[2,3-d]pyrimidin-7-yl)propanamide (10.3 mg, 25.8%) as a white amorphous solid.

### Example 12

### 1-(4-((4-amino-7-(1-hydroxypropan-2-yl)-5-(4-phenoxyphenyl)-7H-pyrrolo[2,3-d]pyrimidin-6-yl)ethynyl)piperidin-1-yl)prop-2-en-1-one

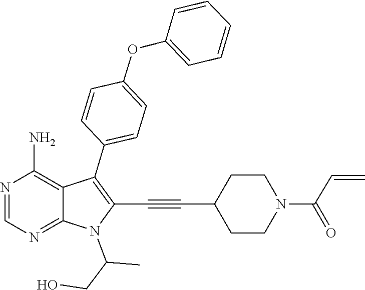 [SMILES: C=CC(=O)N1CCC(C#Cc2c(-c3ccc(Oc4ccccc4)cc3)c3c(N)ncnc3n2C(C)CO)CC1]

### (R)-1-(4-((4-amino-7-(1-hydroxypropan-2-yl)-5-(4-phenoxyphenyl)-7H-pyrrolo[2,3-d]pyrimidin-6-yl)ethynyl)piperidin-1-yl)prop-2-en-1-one, and

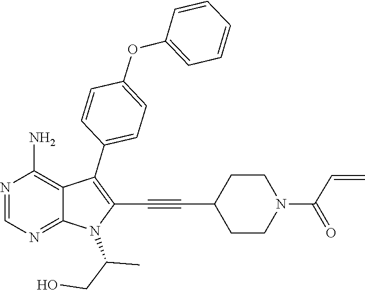 [SMILES: C=CC(=O)N1CCC(C#Cc2c(-c3ccc(Oc4ccccc4)cc3)c3c(N)ncnc3n2[C@H](C)CO)CC1]

### (S)-1-(4-((4-amino-7-(1-hydroxypropan-2-yl)-5-(4-phenoxyphenyl)-7H-pyrrolo[2,3-d]pyrimidin-6-yl)ethynyl)piperidin-1-yl)prop-2-en-1-one

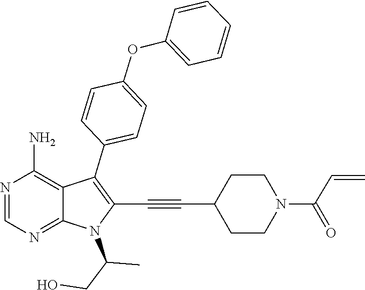 [SMILES: C=CC(=O)N1CCC(C#Cc2c(-c3ccc(Oc4ccccc4)cc3)c3c(N)ncnc3n2[C@@H](C)CO)CC1]

### tert-butyl 4-((4-amino-7-(1-hydroxypropan-2-yl)-5-(4-phenoxyphenyl)-7H-pyrrolo[2,3-d]pyrimidin-6-yl)ethynyl)piperidine-1-carboxylate

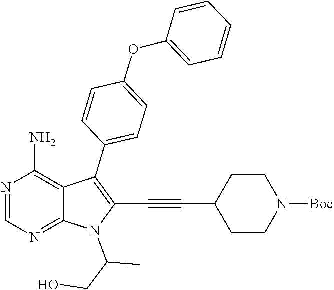 [SMILES: CC(CO)n1c(C#CC2CCN(C(=O)OC(C)(C)C)CC2)c(-c2ccc(Oc3ccccc3)cc2)c2c(N)ncnc21]

### Step 9

LiAlH₄ (88.9 mg, 2.34 mmol) was added to tert-butyl 4-{2-[4-amino-7-(1-methoxy-1-oxopropan-2-yl)-5-(4-phenoxyphenyl)-7H-pyrrolo[2,3-d]pyrimidin-6-yl]ethynyl}piperidine-1-carboxylate (700 mg, 1.17 mmol) in THF (10 mL) at 25° C. The resulting solution was stirred at RT for 2 hours. The crude product was purified by flash C18-flash chromatography, elution gradient 0 to 100% MeCN in water. Pure fractions were evaporated to dryness to afford tert-butyl 4-{2-[4-amino-7-(1-hydroxypropan-2-yl)-5-(4-phenoxyphenyl)-7H-pyrrolo[2,3-d]pyrimidin-6-yl]ethynyl}piperidine-1-carboxylate (480 mg, 72.2%) as a off-white amorphous solid.

### 2-(4-amino-5-(4-phenoxyphenyl)-6-(piperidin-4-ylethynyl)-7H-pyrrolo[2,3-d]pyrimidin-7-yl)propan-1-ol

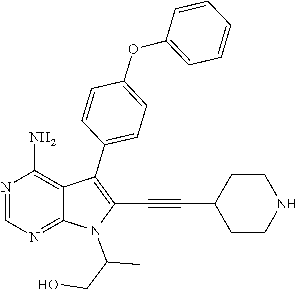 [SMILES: CC(CO)n1c(C#CC2CCNCC2)c(-c2ccc(Oc3ccccc3)cc2)c2c(N)ncnc21]

### Step 10

TFA (2 mL, 845 μmol) was added to tert-butyl 4-{2-[4-amino-7-(1-hydroxypropan-2-yl)-5-(4-phenoxyphenyl)-7H-pyrrolo[2,3-d]pyrimidin-6-yl]ethynyl}piperidine-1-carboxylate (480 mg, 845 μmol) in DCM (8 mL) at 25° C. The resulting solution was stirred at RT for 1 hour. The solvent was removed under reduced pressure. The product was used in the next step directly without further purification.

### 1-(4-((4-amino-7-(1-hydroxypropan-2-yl)-5-(4-phenoxyphenyl)-7H-pyrrolo[2,3-d]pyrimidin-6-yl)ethynyl)piperidin-1-yl)prop-2-en-1-one

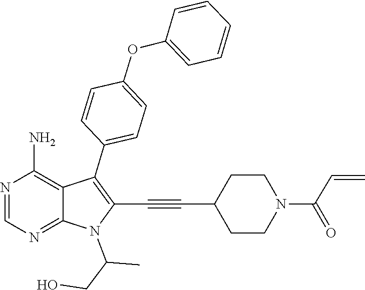 [SMILES: C=CC(=O)N1CCC(C#Cc2c(-c3ccc(Oc4ccccc4)cc3)c3c(N)ncnc3n2C(C)CO)CC1]

### Step 11

prop-2-enoyl chloride (63.8 mg, 705 μmol) was added to 2-[4-amino-5-(4-phenoxyphenyl)-6-[2-(piperidin-4-yl)ethynyl]-7H-pyrrolo[2,3-d]pyrimidin-7-yl]propan-1-ol (330 mg, 705 μmol) and TEA (284 mg, 2.82 mmol) in DCM (8 mL) at 25° C. The resulting solution was stirred at RT for 1 hour. The crude product was purified by preparative HPLC (Column: XBridge Prep C18 OBD Column 19×150 mm 5 um; Mobile Phase A:Water (10 MMOL/L NH₄HCO₃), Mobile Phase B: ACN; Flow rate: 20 mL/min; Gradient: 30% B to 42% B in 16 min; 254&220 nm; Rt: 16.5 min). Fractions containing the desired compound were evaporated to dryness to afford 1-(4-{2-[4-amino-7-(1-hydroxypropan-2-yl)-5-(4-phenoxyphenyl)-7H-pyrrolo[2,3-d]pyrimidin-6-yl]ethynyl}piperidin-1-yl)prop-2-en-1-one (92.5 mg, 25.2%) as a white amorphous solid.

### (R)-1-(4-((4-amino-7-(1-hydroxypropan-2-yl)-5-(4-phenoxyphenyl)-7H-pyrrolo[2,3-d]pyrimidin-6-yl)ethynyl)piperidin-1-yl)prop-2-en-1-one (S)-1-(4-((4-amino-7-(1-hydroxypropan-2-yl)-5-(4-phenoxyphenyl)-7H-pyrrolo[2,3-d]pyrimidin-6-yl)ethynyl)piperidin-1-yl)prop-2-en-1-one

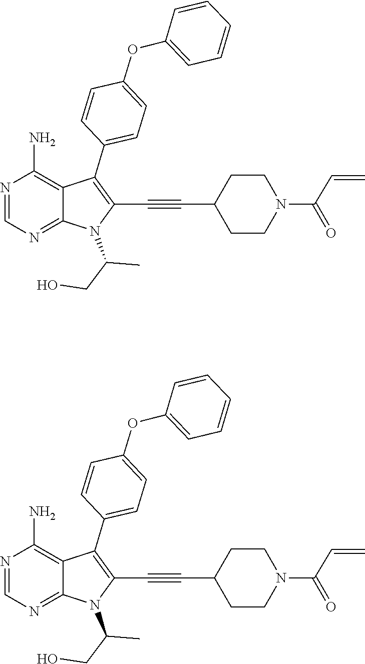 [SMILES: C=CC(=O)N1CCC(C#Cc2c(-c3ccc(Oc4ccccc4)cc3)c3c(N)ncnc3n2[C@H](C)CO)CC1 ; C=CC(=O)N1CCC(C#Cc2c(-c3ccc(Oc4ccccc4)cc3)c3c(N)ncnc3n2[C@@H](C)CO)CC1]

### Step 12

1-(4-{2-[4-amino-7-(1-hydroxypropan-2-yl)-5-(4-phenoxyphenyl)-7H-pyrrolo[2,3-d]pyrimidin-6-yl]ethynyl}piperidin-1-yl)prop-2-en-1-one (50 mg, 95.8 μmol) was purified by preparative chiral-HPLC on a Column: CHIRAL ART Cellulose-SB S-5 um, 2*25 cm, 5 um; Mobile Phase A:Hex (0.1% DEA)-HPLC, Mobile Phase B: EtOH-HPLC; Flow rate: 20 mL/min; Gradient: 25 B to 25 B in 21 min; 220/254 nm; RT1:15.833; RT2:18.018 The fractions containing the desired compound were evaporated to dryness to afford (R)-1-(4-((4-amino-7-(1-hydroxypropan-2-yl)-5-(4-phenoxyphenyl)-7H-pyrrolo[2,3-d]pyrimidin-6-yl)ethynyl)piperidin-1-yl)prop-2-en-1-one (11.2 mg, 22.4%) and (S)-1-(4-((4-amino-7-(1-hydroxypropan-2-yl)-5-(4-phenoxyphenyl)-7H-pyrrolo[2,3-d]pyrimidin-6-yl)ethynyl)piperidin-1-yl)prop-2-en-1-one (14.7 mg, 29.4%) as a white amorphous solid.

Compounds prepared by substantially the same methods, and their characterization data, are listed in Table 9, below.

TABLE 9
Selected Compounds

| | | Proton NMR & |
|---|---|---|
| Compound | Structure | Mass Spec. (obs.) |
| 1-(4-((4-amino-7-(1- hydroxypropan-2- yl)-5-(4- phenoxyphenyl)-7H- pyrrolo[2,3- d]pyrimidin-6- yl)ethynyl)piperidin- 1-yl)prop-2-en-1-one | 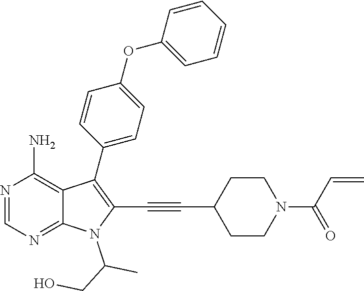 [SMILES: C=CC(=O)N1CCC(C#Cc2c(-c3ccc(Oc4ccccc4)cc3)c3c(N)ncnc3n2C(C)CO)CC1] | ¹H NMR (400 MHz, DMSO-d₆) δ 8.15 (s, 1H), 7.52 − 7.49 (m, 2H), 7.45 − 7.41 (m, 2H), 7.20 − 7.16 (m, 1H), 7.11 − 7.09 (m, 4H), 6.79 (dd, J = 16.7, 10.5 Hz, 1H), 6.09 (dd, J = 16.7, 2.4 Hz, 2H), 5.67 (dd, J = 10.5, 2.4 Hz, 1H), 5.04 (t, J = 5.7 Hz, 1H), 4.91 (q, J = 7.0 Hz, 1H), 4.07 (dt, J = 10.9, 7.5 Hz, 1H), 3.86 (dt, J = 11.0, 5.6 Hz, 1H), 3.64 (s, 2H), 3.44 − 3.38 (m, 2H), 3.02 (dt, J = 7.8, 3.8 Hz, 1H), 1.80 (s, 2H), 1.53 (d, J = 7.0 Hz, 5H). MS: 522.25 |
| 2-(6-((1- acryloylpiperidin-4- yl)ethynyl)-4-amino- 5-(4- phenoxyphenyl)-7H- pyrrolo[2,3- d]pyrimidin-7- yl)propanamide | 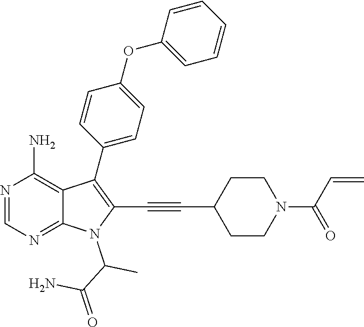 [SMILES: C=CC(=O)N1CCC(C#Cc2c(-c3ccc(Oc4ccccc4)cc3)c3c(N)ncnc3n2C(C)C(N)=O)CC1] | ¹H NMR (400 MHz, DMSO-d₆) δ 8.16 (s, 1H), 7.52 (d, J = 8.6 Hz, 2H), 7.48 − 7.39 (m, 2H), 7.26 (s, 2H), 7.18 (t, J = 7.4 Hz, 1H), 7.15 − 7.07 (m, 4H), 6.79 (dd, J = 16.7, 10.5 Hz, 1H), 6.09 (dd, J = 16.7, 2.4 Hz, 1H), 5.67 (dd, J = 10.5, 2.4 Hz, 1H), 5.43 (q, J = 7.1 Hz, 1H), 3.65 (s, 2H), 3.44 − 3.38 (m, 2H), 2.97 (dd, J = 7.7, 3.9 Hz, 1H), 1.73 (d, J = 7.3 Hz, 5H), 1.53 (s, 2H). MS: 535.35 |
| methyl 2-(6-((1- acryloylpiperidin-4- yl)ethynyl)-4-amino- 5-(4- phenoxyphenyl)-7H- pyrrolo[2,3- d]pyrimidin-7- yl)propanoate | 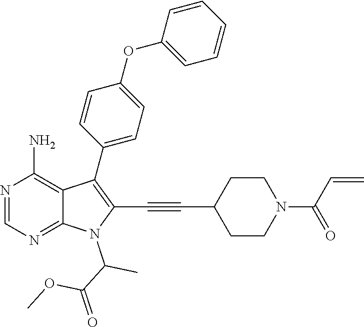 [SMILES: C=CC(=O)N1CCC(C#Cc2c(-c3ccc(Oc4ccccc4)cc3)c3c(N)ncnc3n2C(C)C(=O)OC)CC1] | ¹H NMR (300 MHz, DMSO-d₆) δ 8.16 (s, 1H), 7.54 (d, J = 8.6 Hz, 2H), 7.50 − 7.38 (m, 2H), 7.25 − 7.07 (m, 5H), 6.81 (dd, J = 16.7, 10.5 Hz, 1H), 6.32 (s, 1H), 6.10 (dd, J = 16.7, 2.5 Hz, 1H), 5.75 − 5.64 (m, 2H), 3.66 (s, 5H), 3.44 − 3.38 (m, 2H), 2.99 (t, J = 3.9 Hz, 1H), 1.80 (s, 2H), 1.70 (d, J = 7.1 Hz, 3H), 1.52 (s, 2H). MS: 550.2 |
| 2-(6-((1- acryloylpiperidin-4- yl)ethynyl)-4-amino- 5-(4- phenoxyphenyl)-7H- pyrrolo[2,3- d]pyrimidin-7- yl)propanoic acid | 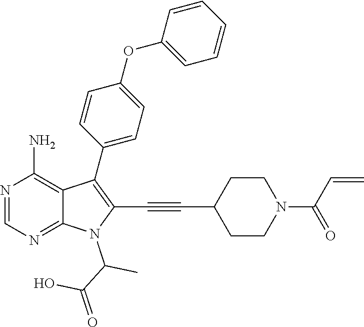 [SMILES: C=CC(=O)N1CCC(C#Cc2c(-c3ccc(Oc4ccccc4)cc3)c3c(N)ncnc3n2C(C)C(=O)O)CC1] | ¹H NMR (400 MHz, DMSO-d₆) δ 8.14 (s, 1H), 7.52 − 7.50 (m, 2H), 7.44 − 7.40 (m, 2H), 7.17 (t, J = 7.4 Hz, 1H), 7.11 − 7.09 (m, 4H), 6.78 (dd, J = 16.7, 10.4 Hz, 1H), 6.06 (dd, J = 16.7, 2.5 Hz, 1H), 5.66 (dd, J = 10.5, 2.5 Hz, 1H), 5.43 (s, 1H), 3.65 (s, 2H), 3.54 − 3.50 (m, 2H), 2.98 − 2.94 (m, 1H), 1.76 − 1.70 (m, 5H), 1.53 (s, 2H). MS: 536.3 |
| methyl 2-(4-amino- 5-(4- phenoxyphenyl)-6- (piperidin-4- ylethynyl)-7H- pyrrolo[2,3- d]pyrimidin-7- yl)propanoate | 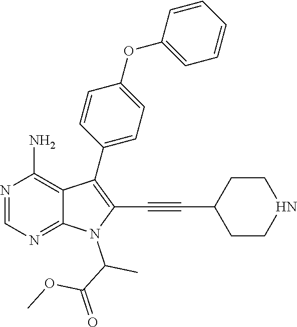 [SMILES: COC(=O)C(C)n1c(C#CC2CCNCC2)c(-c2ccc(Oc3ccccc3)cc2)c2c(N)ncnc21] | ¹H NMR (300 MHz, DMSO-d₆) δ 8.16 (s, 1H), 7.54 − 7.50 (m, 2H), 7.45 − 7.40 (m, 2H), 7.20 − 7.10 (m, 5H), 6.28 (s, 1H), 5.69 (q, J = 7.0 Hz, 1H), 3.65 (s, 3H), 2.86 − 2.71 (m, 3H), 2.55 (s, 2H), 1.70-1.68 (m, 5H), 1.48 − 1.41 (m, 2H). MS: 496.2 |
| (R)-1-(4-((4-amino- 7-(1-hydroxypropan- 2-yl)-5-(4- phenoxyphenyl)-7H- pyrrolo[2,3- d]pyrimidin-6- yl)ethynyl)piperidin- 1-yl)prop-2-en-1-one | 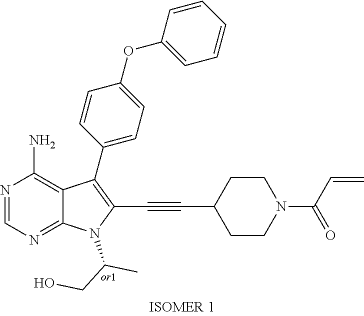 [SMILES: C=CC(=O)N1CCC(C#Cc2c(-c3ccc(Oc4ccccc4)cc3)c3c(N)ncnc3n2[C@H](C)CO)CC1] | ¹H NMR (300 MHz, Methanol-d4) δ 8.16 (s, 1H), 7.54 − 7.51 (m, 2H), 7.44 − 7.39 (m, 2H), 7.20 − 7.07 (m, 5H), 6.77 (dd, J = 16.8, 10.6 Hz, 1H), 6.22 (dd, J = 16.8, 2.0 Hz, 1H), 5.74 (dd, J = 10.6, 2.0 Hz, 1H), 5.04 (td, J = 7.5, 5.1 Hz, 1H), 4.30 (dd, J = 11.6, 8.0 Hz, 1H), 4.01 (dd, J = 11.5, 5.0 Hz, 1H), 3.68 − 3.59 (m, 4H), 3.07 (tt, J = 7.3, 4.1 Hz, 1H), 1.88 (s, 2H), 1.70 − 1.62 (m, 5H). MS: 522.2 |
| (S)-1-(4-((4-amino- 7-(1-hydroxypropan- 2-yl)-5-(4- phenoxyphenyl)-7H- pyrrolo[2,3- d]pyrimidin-6- yl)ethynyl)piperidin- 1-yl)prop-2-en-1-one | 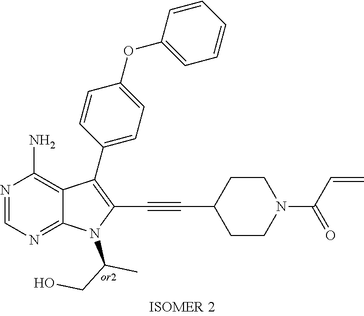 [SMILES: C=CC(=O)N1CCC(C#Cc2c(-c3ccc(Oc4ccccc4)cc3)c3c(N)ncnc3n2[C@@H](C)CO)CC1] | ¹H NMR (300 MHz, Methanol-d4) δ 8.13 (s, 1H), 7.51 − 7.48 (m, 2H), 7.41 − 7.36 (m, 2H), 7.17 − 7.03 (m, 5H), 6.73 (dd, J = 16.8, 10.7 Hz, 1H), 6.20 (dd, J = 16.8, 2.0 Hz, 1H), 5.72 (dd, J = 10.6, 2.0 Hz, 1H), 5.00 (td, J = 7.5, 5.1 Hz, 1H), 4.26 (dd, J = 11.5, 8.0 Hz, 1H), 3.98 (dd, J = 11.6, 5.0 Hz, 1H), 3.65 − 3.55 (m, 4H), 3.03 (tt, J = 7.4, 4.1 Hz, 1H), 1.86 (s, 2H), 1.67 − 1.58 (m, 5H). MS: 522.2 |
| (E)-1-(3-((4-amino- 7-(1-hydroxypropan- 2-yl)-5-(4- phenoxyphenyl)-7H- pyrrolo[2,3- d]pyrimidin-6- yl)ethynyl) pyrrolidin- 1-yl)-4- (dimethylamino)but- 2-en-1-one | 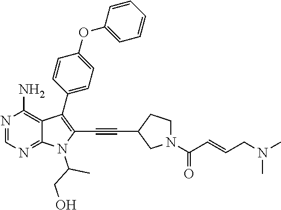 [SMILES: CC(CO)n1c(C#CC2CCN(C(=O)/C=C/CN(C)C)C2)c(-c2ccc(Oc3ccccc3)cc2)c2c(N)ncnc21] | ¹H NMR (400 MHz, DMSO-d₆) δ 8.15 (s, 1H), 7.50 − 7.44 (m, 4H), 7.12 (dt, J = 26.5, 8.0 Hz, 5H), 6.64 (d, J = 12.7 Hz, 1H), 6.60 (d, J = 15.3 Hz, 1H), 6.24 (d, 1H), 5.02 (s, 1H), 4.89 (s, 1H), 4.05 (s, 1H), 3.82 (d, J = 8.3 Hz, 2H), 3.60 (d, J = 7.6 Hz, 2H), 3.00 − 2.98(m, 2H), 2.23 (s, 1H), 2.12 (s, 6H), 1.96 (s, 1H), 1.51 (d, J = 7.0 Hz, 3H). MS: 565.30 |
| 1-(3-((4-amino-7-(1- hydroxypropan-2- yl)-5-(4- phenoxyphenyl)-7H- pyrrolo[2,3- d]pyrimidin-6- yl)ethynyl)pyrrolidin- 1-yl)prop-2-en-1- one | 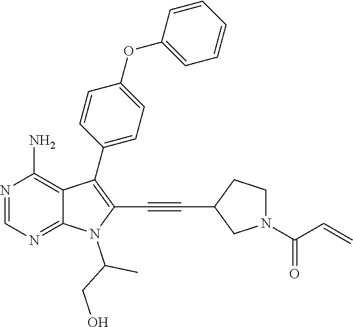 [SMILES: C=CC(=O)N1CCC(C#Cc2c(-c3ccc(Oc4ccccc4)cc3)c3c(N)ncnc3n2C(C)CO)C1] | ¹H NMR (400 MHz, DMSO-d₆) δ 8.15 (s, 1H), 7.47 − 7.42 (m, 4H), 7.15 (dt, J = 28.9, 8.6 Hz, 5H), 6.58 − 6.52 (m, 1H), 6.14 (d, J = 16.8 Hz, 1H), 5.65 (t, J = 9.7 Hz, 1H), 5.02 (s, 1H), 5.02 (s, 1H), 4.90 (s, 1H), 4.05 (s, 1H), 3.84 (d, J = 9.2 Hz, 1H), 3.65 − 3.61 (m, 2H), 3.54 − 3.52 (m, 2H), 2.23 (d, J = 33.0 Hz, 1H), 2.01 (d, J = 33.8 Hz, 1H), 1.51 (d, J = 7.0 Hz, 3H). MS: 508.25 |
| (E)-1-(4-((4-amino- 7-(1-hydroxypropan- 2-yl)-5-(4- phenoxyphenyl)-7H- pyrrolo[2,3- d]pyrimidin-6- yl)ethynyl)-4- hydroxypiperidin-1- yl)-4- (dimethylamino)but- 2-en-1-one | 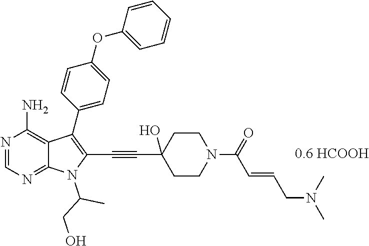 [SMILES: CC(CO)n1c(C#CC2(O)CCN(C(=O)/C=C/CN(C)C)CC2)c(-c2ccc(Oc3ccccc3)cc2)c2c(N)ncnc21] | ¹H NMR (400 MHz, DMSO-d₆) δ 8.16 (s, 2H), 7.52 − 7.50 (m, 2H), 7.45 − 7.41 (m, 2H), 7.18 (t, J = 7.4 Hz, 1H), 7.16 − 7.11 (m, 4H), 6.67 − 6.55 (m, 2H), 6.19 (br, 1H), 5.86 (s, 1H), 4.99 (d, J = 18.2 Hz, 1H), 4.94 (q, J = 7.1 Hz, 1H), 4.08 (t, J = 9.5 Hz, 1H), 3.87 (dd, J = 11.1, 6.0 Hz, 2H), 3.75 (d, J = 13.5 Hz, 1H), 3.32 − 3.29 (m, 1H), 3.18 (d, J = 5.8 Hz, 3H), 2.27 (s, 6H), 1.82 (s, 2H), 1.62 (s, 2H), 1.55 (d, J = 7.0 Hz, 3H). MS: 595.25 |

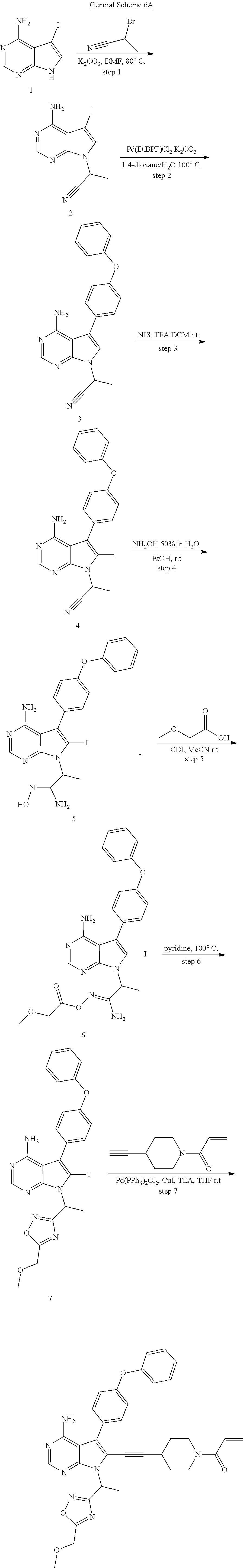

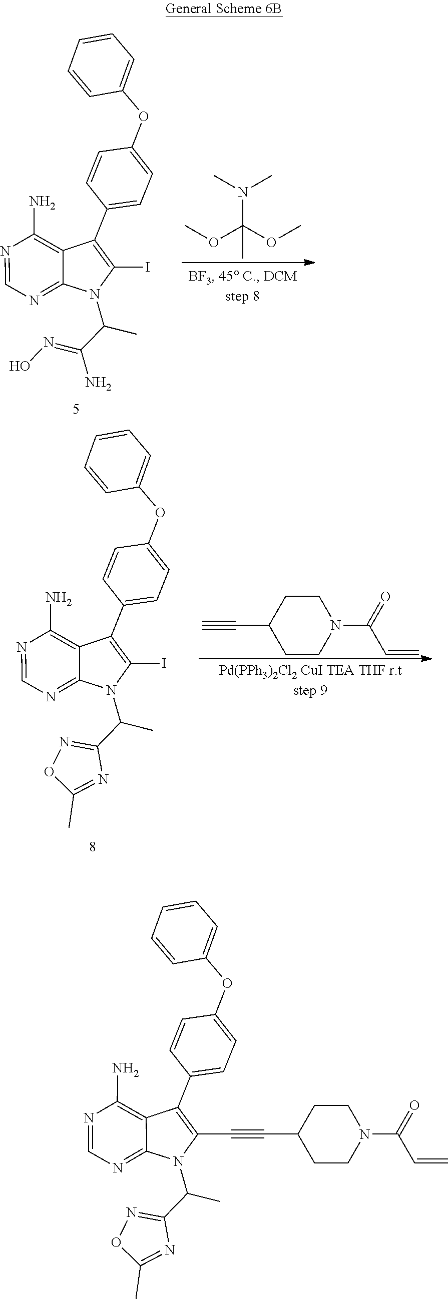

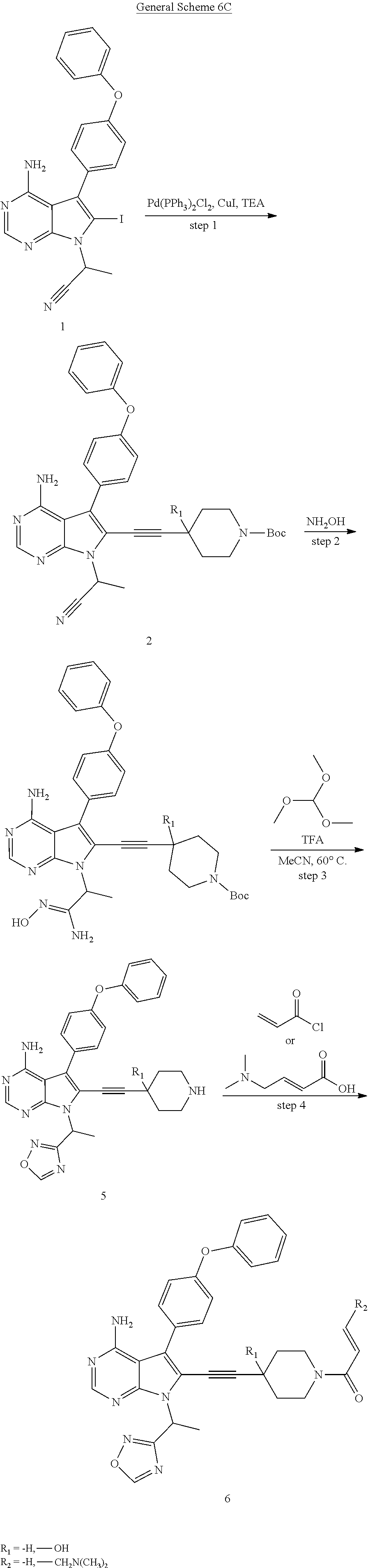

### Example 13

### 1-(4-((4-amino-7-(1-(5-(methoxymethyl)-1,2,4-oxadiazol-3-yl)ethyl)-5-(4-phenoxyphenyl)-7H-pyrrolo[2,3-d]pyrimidin-6-yl)ethynyl)piperidin-1-yl)prop-2-en-1-one

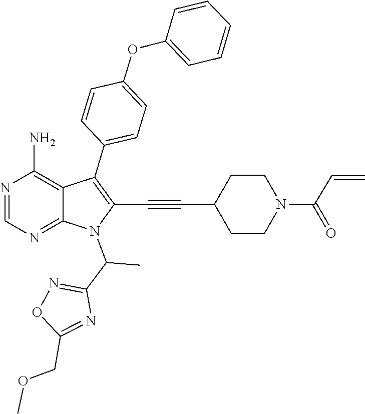 [SMILES: C=CC(=O)N1CCC(C#Cc2c(-c3ccc(Oc4ccccc4)cc3)c3c(N)ncnc3n2C(C)c2noc(COC)n2)CC1]

### 2-(4-amino-5-iodo-7H-pyrrolo[2,3-d]pyrimidin-7-yl)propanenitrile

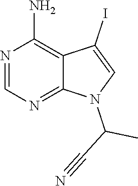

### Step 1

2-bromopropanenitrile (1.23 g, 9.22 mmol) was added to 5-iodo-7H-pyrrolo[2,3-d]pyrimidin-4-amine (2 g, 7.69 mmol) and K₂CO₃ (3.17 g, 23.0 mmol) in DMF (10 mL) at r.t. The resulting solution was stirred at 80° C. for 2 hours. The reaction mixture was diluted with H₂O (100 mL), and the aqueous phase was extracted with ethyl acetate (400 mL) three times. The combined organic layers were washed with saturated brines, dried over sodium sulfate, filtered, and concentrated in vacuo. The crude product was purified by flash C18-flash chromatography, elution gradient 0 to 100% MeCN in water. Pure fractions were evaporated to dryness to afford 2-{4-amino-5-iodo-7H-pyrrolo[2,3-d]pyrimidin-7-yl}propanenitrile (1.80 g, 75%) as a yellow amorphous solid.

### 2-(4-amino-5-(4-phenoxyphenyl)-7H-pyrrolo[2,3-d]pyrimidin-7-yl)propanenitrile

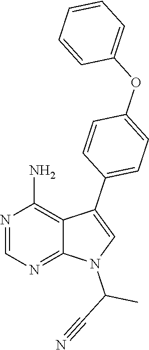 [SMILES: CC(C#N)n1cc(-c2ccc(Oc3ccccc3)cc2)c2c(N)ncnc21]

### Step 2

Pd(DtBPF)Cl₂ (540 mg, 830 μmol) was added to 2-{4-amino-5-iodo-7H-pyrrolo[2,3-d]pyrimidin-7-yl}propanenitrile (1.3 g, 4.15 mmol), (4-phenoxyphenyl)boronic acid (1.77 g, 8.30 mmol) and K₂CO₃ (1.71 g, 12.4 mmol) in water (2 mL) and 1,4-dioxane (16 mL) at RT under nitrogen. The resulting solution was stirred at 100° C. for 2 hours. The reaction mixture was diluted with H₂O (50 mL), and the aqueous phase was extracted with ethyl acetate (300 mL) three times. The combined organic layers were washed with saturated brines, dried over sodium sulfate, filtered, and concentrated in vacuo. The crude product was purified by flash C18-flash chromatography, elution gradient 0 to 100% MeCN in water. Pure fractions were evaporated to dryness to afford 2-[4-amino-5-(4-phenoxyphenyl)-7H-pyrrolo[2,3-d]pyrimidin-7-yl]propanenitrile (1.20 g, 81.6%) as a yellow amorphous solid.

### 2-(4-amino-6-iodo-5-(4-phenoxyphenyl)-7H-pyrrolo[2,3-d]pyrimidin-7-yl)propanenitrile

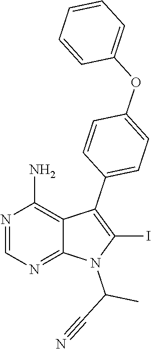 [SMILES: CC(C#N)n1c(I)c(-c2ccc(Oc3ccccc3)cc2)c2c(N)ncnc21]

### Step 3

TFA (768 mg, 6.74 mmol) was added to 2-[4-amino-5-(4-phenoxyphenyl)-7H-pyrrolo[2,3-d]pyrimidin-7-yl]propanenitrile (1.2 g, 3.37 mmol) and NIS (754 mg, 3.37 mmol) in DCM (20 mL) at RT. The resulting solution was stirred at RT for 1 hour. The solvent was removed under reduced pressure. The crude product was purified by flash C18-flash chromatography, elution gradient 0 to 100% MeCN in water. Pure fractions were evaporated to dryness to afford 2-[4-amino-6-iodo-5-(4-phenoxyphenyl)-7H-pyrrolo[2,3-d]pyrimidin-7-yl]propanenitrile (600 mg, 37%) as a white amorphous solid.

### (Z)-2-(4-amino-6-iodo-5-(4-phenoxyphenyl)-7H-pyrrolo[2,3-d]pyrimidin-7-yl)-N′-hydroxypropanimidamide

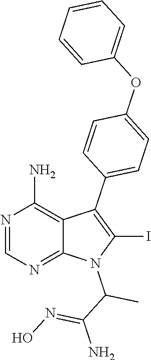 [SMILES: CC(/C(N)=N/O)n1c(I)c(-c2ccc(Oc3ccccc3)cc2)c2c(N)ncnc21]

### Step 4

Hydroxy amine solution (50% in water) (205 mg, 6.22 mmol) was added to 2-[4-amino-6-iodo-5-(4-phenoxyphenyl)-7H-pyrrolo[2,3-d]pyrimidin-7-yl]propanenitrile (300 mg, 623 μmol) in EtOH (6 mL) at RT. The resulting solution was stirred at RT for 4 hours. The solvent was removed under reduced pressure. The product was used in the next step directly without further purification.

### (Z)-2-(4-amino-6-iodo-5-(4-phenoxyphenyl)-7H-pyrrolo[2,3-d]pyrimidin-7-yl)-N′-(2-methoxyacetoxy)propanimidamide

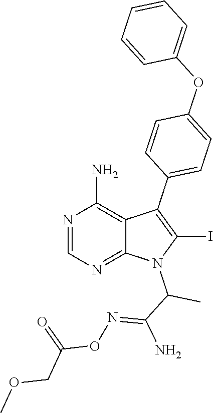 [SMILES: COCC(=O)O/N=C(\N)C(C)n1c(I)c(-c2ccc(Oc3ccccc3)cc2)c2c(N)ncnc21]

### Step 5

To a stirred solution of CDI (113 mg, 699 μmol) in MeCN 2-methoxyacetic acid (57.7 mg, 641 μmol) was added. The mixture was stirred at 45° C. for 1 h, and then (Z)-2-[4-amino-6-iodo-5-(4-phenoxyphenyl)-7H-pyrrolo[2,3-d]pyrimidin-7-yl]-N′-hydroxypropanimidamide (300 mg, 583 μmol) was added. The reaction mixture was stirred 3 hours at room temperature. The crude product was purified by flash silica chromatography, elution gradient 25% MeOH in DCM. Pure fractions were evaporated to dryness to afford (Z)-{1-amino-2-[4-amino-6-iodo-5-(4-phenoxyphenyl)-7H-pyrrolo[2,3-d]pyrimidin-7-yl]propylidene}amino 2-methoxyacetate (300 mg, 87.9%) as a white amorphous solid.

### 6-iodo-7-(1-(5-(methoxymethyl)-1,2,4-oxadiazol-3-yl)ethyl)-5-(4-phenoxyphenyl)-7H-pyrrolo[2,3-d]pyrimidin-4-amine

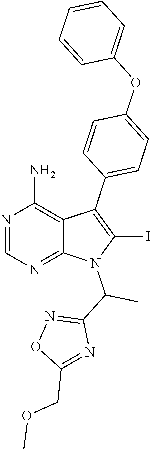 [SMILES: COCc1nc(C(C)n2c(I)c(-c3ccc(Oc4ccccc4)cc3)c3c(N)ncnc32)no1]

### Step 6

To (Z)-{1-amino-2-[4-amino-6-iodo-5-(4-phenoxyphenyl)-7H-pyrrolo[2,3-d]pyrimidin-7-yl]propylidene}amino 2-methoxyacetate (200 mg, 341 μmol) pyridine (3 mL) was added. The reaction mixture was stirred at 100° C. for 5 hours, then cooled to room temperature, and concentrated. The crude product was purified by flash C18-flash chromatography, elution gradient 0 to 100% MeCN in water. Pure fractions were evaporated to dryness to afford 6-iodo-7-{1-[5-(methoxymethyl)-1,2,4-oxadiazol-3-yl]ethyl}-5-(4-phenoxyphenyl)-7H-pyrrolo[2,3-d]pyrimidin-4-amine (120 mg, 62.1%) as a off-white amorphous solid.

### 1-(4-((4-amino-7-(1-(5-(methoxymethyl)-1,2,4-oxadiazol-3-yl)ethyl)-5-(4-phenoxyphenyl)-7H-pyrrolo[2,3-d]pyrimidin-6-yl)ethynyl)piperidin-1-yl)prop-2-en-1-one

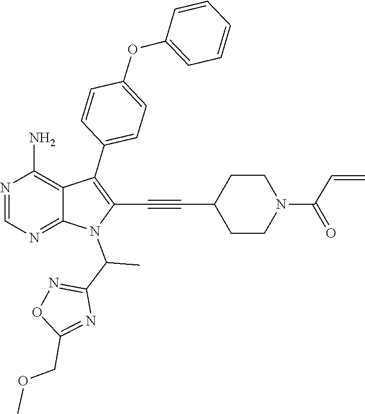 [SMILES: C=CC(=O)N1CCC(C#Cc2c(-c3ccc(Oc4ccccc4)cc3)c3c(N)ncnc3n2C(C)c2noc(COC)n2)CC1]

### Step 7

TEA (1 mL, 122 μmol) was added to 6-iodo-7-{1-[5-(methoxymethyl)-1,2,4-oxadiazol-3-yl]ethyl}-5-(4-phenoxyphenyl)-7H-pyrrolo[2,3-d]pyrimidin-4-amine (70 mg, 123 μmol), Pd(PPh₃)₂Cl₂ (25.8 mg, 36.9 μmol), 1-(4-ethynylpiperidin-1-yl)prop-2-en-1-one (39.9 mg, 245 μmol) and CuI (9.32 mg, 49.1 μmol) in THE (2 mL) at RT under nitrogen. The resulting solution was stirred at RT for 4 hours. The crude product was purified by preparative HPLC (Column: XBridge Shield RP18 OBD Column, 5 um, 19*150 mm; Mobile Phase A:Water (10 MMOL/L NH₄HCO₃), Mobile Phase B: ACN; Flow rate: 20 mL/min; Gradient: 43% B to 62% B in 7 min; 254 nm; Rt: 6.12 min). Fractions containing the desired compound were evaporated to dryness to afford 1-{4-[2-(4-amino-7-{1-[5-(methoxymethyl)-1,2,4-oxadiazol-3-yl]ethyl}-5-(4-phenoxyphenyl)-7H-pyrrolo[2,3-d]pyrimidin-6-yl)ethynyl]piperidin-1-yl}prop-2-en-1-one (24.1 mg, 32.7%) as a white amorphous solid.

### Example 14

### 1-(4-((4-amino-7-(1-(5-methyl-1,2,4-oxadiazol-3-yl)ethyl)-5-(4-phenoxyphenyl)-7H-pyrrolo[2,3-d]pyrimidin-6-yl)ethynyl)piperidin-1-yl)prop-2-en-1-one

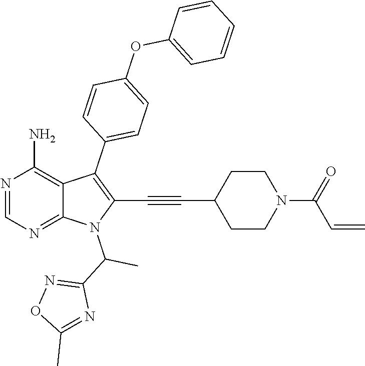 [SMILES: C=CC(=O)N1CCC(C#Cc2c(-c3ccc(Oc4ccccc4)cc3)c3c(N)ncnc3n2C(C)c2noc(C)n2)CC1]

### 6-iodo-7-(1-(5-methyl-1,2,4-oxadiazol-3-yl)ethyl)-5-(4-phenoxyphenyl)-7H-pyrrolo[2,3-d]pyrimidin-4-amine

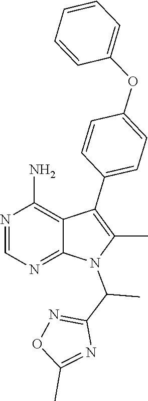 [SMILES: Cc1nc(C(C)n2c(C)c(-c3ccc(Oc4ccccc4)cc3)c3c(N)ncnc32)no1]

### Step 8

To a stirred solution of (Z)-2-[4-amino-6-iodo-5-(4-phenoxyphenyl)-7H-pyrrolo[2,3-d]pyrimidin-7-yl]-N′-hydroxypropanimidamide (180 mg, 349 μmol) in DCM (2 mL) (1,1-dimethoxyethyl)dimethylamine (185 mg, 1.39 mmol), boron trifluoride etherate (49.5 mg, 349 μmol) were added. The reaction mixture was stirred at 45° C. for 4 hours. The solvent was removed under reduced pressure. The crude product was purified by flash C18-flash chromatography, elution gradient 0 to 100% MeCN in water. Pure fractions were evaporated to dryness to afford 6-iodo-7-[1-(5-methyl-1,2,4-oxadiazol-3-yl)ethyl]-5-(4-phenoxyphenyl)-7H-pyrrolo[2,3-d]pyrimidin-4-amine (140 mg, 74.8%) as a white amorphous solid.

### 1-(4-((4-amino-7-(1-(5-methyl-1,2,4-oxadiazol-3-yl)ethyl)-5-(4-phenoxyphenyl)-7H-pyrrolo[2,3-d]pyrimidin-6-yl)ethynyl)piperidin-1-yl)prop-2-en-1-one

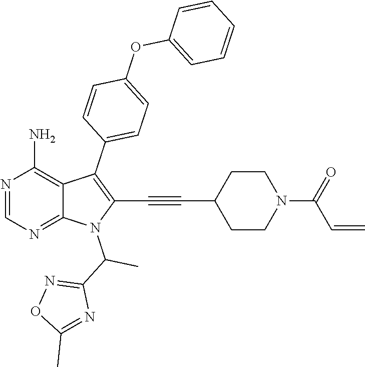 [SMILES: C=CC(=O)N1CCC(C#Cc2c(-c3ccc(Oc4ccccc4)cc3)c3c(N)ncnc3n2C(C)c2noc(C)n2)CC1]

### Step 9

TEA (1 mL, 241 μmol) was added to 6-iodo-7-[1-(5-methyl-1,2,4-oxadiazol-3-yl)ethyl]-5-(4-phenoxyphenyl)-7H-pyrrolo[2,3-d]pyrimidin-4-amine (130 mg, 241 μmol), Pd(PPh₃)₂Cl₂ (50.6 mg, 72.3 μmol), 1-(4-ethynylpiperidin-1-yl)prop-2-en-1-one (78.6 mg, 482 μmol) and CuI (18.3 mg, 96.4 μmol) in THE (2 mL) at RT under nitrogen. The resulting solution was stirred at RT for 4 hours. The crude product was purified by preparative HPLC (Column: XBridge Prep OBD C18 Column 30×150 mm 5 um; Mobile Phase A:Water (10 MMOL/L NH₄HCO₃), Mobile Phase B: ACN; Flow rate: 60 mL/min; Gradient: 30% B to 60% B in 7 min; 220 nm; Rt: 6.8 min). Fractions containing the desired compound were evaporated to dryness to afford 1-[4-(2-{4-amino-7-[1-(5-methyl-1,2,4-oxadiazol-3-yl)ethyl]-5-(4-phenoxyphenyl)-7H-pyrrolo[2,3-d]pyrimidin-6-yl}ethynyl)piperidin-1-yl]prop-2-en-1-one (23.0 mg, 17%) as a white amorphous solid.

### Example 15

### 1-(4-((7-(1-(1,2,4-oxadiazol-3-yl)ethyl)-4-amino-5-(4-phenoxyphenyl)-7H-pyrrolo[2,3-d]pyrimidin-6-yl)ethynyl)piperidin-1-yl)prop-2-en-1-one

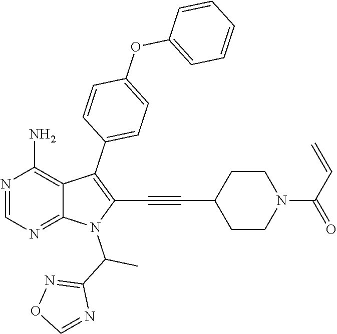 [SMILES: C=CC(=O)N1CCC(C#Cc2c(-c3ccc(Oc4ccccc4)cc3)c3c(N)ncnc3n2C(C)c2ncon2)CC1]

### tert-butyl 4-((4-amino-7-(1-cyanoethyl)-5-(4-phenoxyphenyl)-7H-pyrrolo[2,3-d]pyrimidin-6-yl)ethynyl)piperidine-1-carboxylate

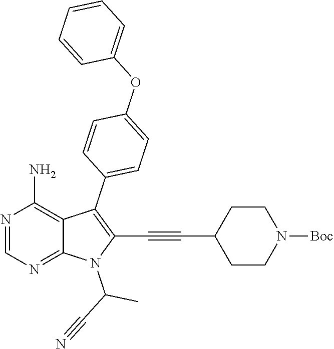 [SMILES: CC(C#N)n1c(C#CC2CCN(C(=O)OC(C)(C)C)CC2)c(-c2ccc(Oc3ccccc3)cc2)c2c(N)ncnc21]

### Step 1

A round bottomed flask was charged with 2-[4-amino-6-iodo-5-(4-phenoxyphenyl)-7H-pyrrolo[2,3-d]pyrimidin-7-yl]propanenitrile (500 mg, 1.03 mmol), tert-butyl 4-ethynylpiperidine-1-carboxylate (322 mg, 1.54 mmol), Pd(PPh₃)₂Cl₂ (144 mg, 206 μmol), CuI (19.5 mg, 103 μmol), TEA (312 mg, 3.09 mmol) and a stirbar in THF (15 mL) under N₂, and the solution was stirred at 25° C. for 16 hours under N₂. The solution was diluted with EA (50 mL) and washed with brine (50 mL). Dried and concentrated. The resulting crude material was purified by FLASH C18 (acetonitrile/water/0.1% formic acid). Concentrated yielded tert-butyl 4-{2-[4-amino-7-(1-cyanoethyl)-5-(4-phenoxyphenyl)-7H-pyrrolo[2,3-d]pyrimidin-6-yl]ethynyl}piperidine-1-carboxylate (400 mg, 710 μmol) as a yellow amorphous solid.

### (Z)-tert-butyl 4-((4-amino-7-(1-amino-1-(hydroxyimino)propan-2-yl)-5-(4-phenoxyphenyl)-7H-pyrrolo[2,3-d]pyrimidin-6-yl)ethynyl)piperidine-1-carboxylate

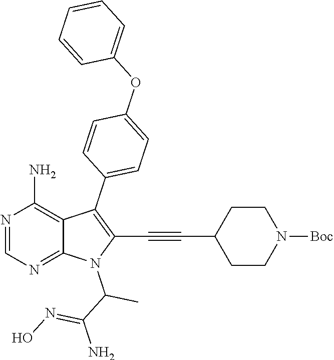 [SMILES: CC(/C(N)=N/O)n1c(C#CC2CCN(C(=O)OC(C)(C)C)CC2)c(-c2ccc(Oc3ccccc3)cc2)c2c(N)ncnc21]

### Step 5

A round bottomed flask was charged with tert-butyl 4-{2-[4-amino-7-(1-cyanoethyl)-5-(4-phenoxyphenyl)-7H-pyrrolo[2,3-d]pyrimidin-6-yl]ethynyl}piperidine-1-carboxylate (400 mg, 710 μmol), hydroxylamine (158 mg, 4.79 mmol, 50% in H₂O), and a stirbar in EtOH (10 mL), and the solution was stirred at 25° C. for 1 hour. Concentrated under vacuum. This resulted in tert-butyl 4-[2-(4-amino-7-{1-[(Z)—N′-hydroxycarbamimidoyl]ethyl}-5-(4-phenoxyphenyl)-7H-pyrrolo[2,3-d]pyrimidin-6-yl)ethynyl]piperidine-1-carboxylate (360 mg, 604 μmol) as yellow solid, used directly for next step with out further purification.

### 7-(1-(1,2,4-oxadiazol-3-yl)ethyl)-5-(4-phenoxyphenyl)-6-(piperidin-4-ylethynyl)-7H-pyrrolo[2,3-d]pyrimidin-4-amine

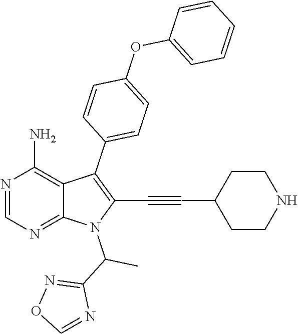 [SMILES: CC(c1ncon1)n1c(C#CC2CCNCC2)c(-c2ccc(Oc3ccccc3)cc2)c2c(N)ncnc21]

### Step 6

A round bottomed flask was charged with tert-butyl 4-[2-(4-amino-7-{1-[(Z)—N′-hydroxycarbamimidoyl]ethyl}-5-(4-phenoxyphenyl)-7H-pyrrolo[2,3-d]pyrimidin-6-yl)ethynyl]piperidine-1-carboxylate (360 mg, 604 μmol), trimethoxymethane (639 mg, 6.03 mmol), TFA (687 mg, 6.03 mmol) and a stirbar in acetonitrile (15 mL), and the solution was stirred at 60° C. for 1 hour. Concentrated under vacuum. The resulting crude material was purified by FLASH C18 (acetonitrile/water/0.1% formic acid). Concentrated yielded 7-[1-(1,2,4-oxadiazol-3-yl)ethyl]-5-(4-phenoxyphenyl)-6-[2-(piperidin-4-yl)ethynyl]-7H-pyrrolo[2,3-d]pyrimidin-4-amine (198 mg, 391 μmol) as a yellow oil.

### 1-(4-((7-(1-(1,2,4-oxadiazol-3-yl)ethyl)-4-amino-5-(4-phenoxyphenyl)-7H-pyrrolo[2,3-d]pyrimidin-6-yl)ethynyl)piperidin-1-yl)prop-2-en-1-one

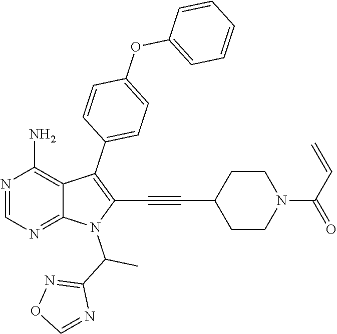 [SMILES: C=CC(=O)N1CCC(C#Cc2c(-c3ccc(Oc4ccccc4)cc3)c3c(N)ncnc3n2C(C)c2ncon2)CC1]

### Step 7

A round bottomed flask was charged with 7-[1-(1,2,4-oxadiazol-3-yl)ethyl]-5-(4-phenoxyphenyl)-6-[2-(piperidin-4-yl)ethynyl]-7H-pyrrolo[2,3-d]pyrimidin-4-amine (190 mg, 375 μmol), TEA (113 mg, 1.12 mmol), and a stirbar in dichloromethane (10 mL), the solution was cooled to 0° C., then, prop-2-enoyl chloride (33.9 mg, 375 μmol) was added slowly, and the solution was stirred at 0° C. for 30 minutes. Concentrated under vacuum at rt. The resulting crude material was purified by HPLC (acetonitrile/water/0.1% formic acid). Lyophilization yielded 1-[4-(2-{4-amino-7-[1-(1,2,4-oxadiazol-3-yl)ethyl]-5-(4-phenoxyphenyl)-7H-pyrrolo[2,3-d]pyrimidin-6-yl}ethynyl)piperidin-1-yl]prop-2-en-1-one (50.0 mg, 89.3 μmol) as a white amorphous solid.

Compounds prepared by substantially the same methods, and their characterization data, are listed in Table 10, below.

TABLE 10
Selected Compounds

| | | Proton NMR & |
|---|---|---|
| Compound | Structure | Mass Spec. (obs.) |
| 1-(4-((7-(1-(1,2,4- oxadiazol-3- yl)ethyl)-4-amino-5- (4-phenoxyphenyl)- 7H-pyrrolo[2,3- d]pyrimidin-6- yl)ethynyl)piperidin- 1-yl)prop-2-en-1-one | 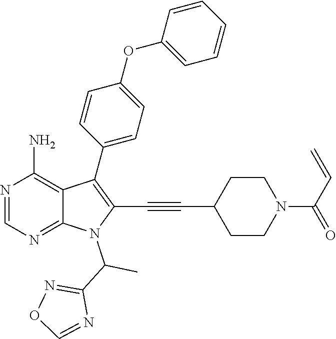 [SMILES: C=CC(=O)N1CCC(C#Cc2c(-c3ccc(Oc4ccccc4)cc3)c3c(N)ncnc3n2C(C)c2ncon2)CC1] | ¹H NMR (400 MHz, DMSO-d₆) δ 9.582 (s, 1H), 8.165 (s, 1H), 7.496-7.515 (d, J = 8.0 Hz, 2H), 7.426-7.444 (t, J = 7.6 Hz, 2H), 7.159- 7.194 (t, J = 7.5 Hz, 1H), 7.093-7.112 (d, J = 8.0 Hz, 4H), 6.747-6.815 (dd, J = 16.6, 10.6 Hz, 1H), 6.326-6.343 (d, J = 6.5 Hz, 1H), 6.064-6.105 (d, J = 16.5 Hz, 1H), 5.650-5.674 (d, J = 10.4 Hz, 1H), 3.616 (s, 2H), 3.286 (s, 2H),2.854 (s, 1H), 1.988-2.004 (d, J = 7.0 Hz, 3H), 1.684-1.695 (s, 2H), 1.370 (s, 2H). MS: 560 |
| 1-(4-((4-amino-7-(1- (5-(methoxymethyl)- 1,2,4-oxadiazol-3- yl)ethyl)-5-(4- phenoxyphenyl)-7H- pyrrolo[2,3- d]pyrimidin-6- yl)ethynyl)piperidin- 1-yl)prop-2-en-1-one | 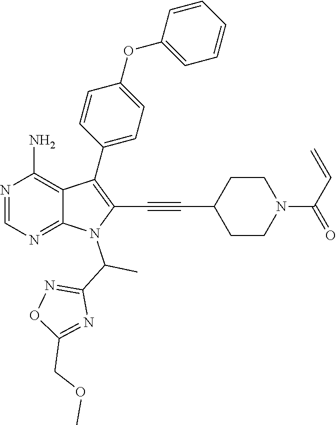 [SMILES: C=CC(=O)N1CCC(C#Cc2c(-c3ccc(Oc4ccccc4)cc3)c3c(N)ncnc3n2C(C)c2noc(COC)n2)CC1] | ¹H NMR (400 MHz, DMSO-d₆) δ 8.17 (s, 1H), 7.51 (d, J = 8.1 Hz, 2H), 7.43 (t, J = 7.7 Hz, 2H), 7.18 (t, J = 7.3 Hz, 1H), 7.10 (d, J = 8.0 Hz, 4H), 6.78 (dd, J = 16.7, 10.5 Hz, 1H), 6.32 (d, J = 7.5 Hz, 1H), 6.09 (d, J = 16.7 Hz, 1H), 5.66 (d, J = 10.5 Hz, 1H), 4.71 (s, 2H), 3.63 (s, 2H), 3.43 − 3.37 (m, 1H), 3.33 − 3.24 (m, 4H), 2.86 (s, 1H), 1.97 (d, J = 6.9 Hz, 3H), 1.70 (s, 2H), 1.36 (d, J = 13.5 Hz, 2H). MS: 604.25 |
| 1-(4-((4-amino-7-(1- (5-methyl-1,2,4- oxadiazol-3- yl)ethyl)-5-(4- phenoxyphenyl)-7H- pyrrolo [2,3- d]pyrimidin-6- yl)ethynyl)piperidin- 1-yl)prop-2-en-1-one | 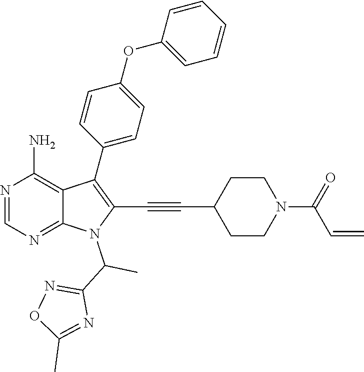 [SMILES: C=CC(=O)N1CCC(C#Cc2c(-c3ccc(Oc4ccccc4)cc3)c3c(N)ncnc3n2C(C)c2noc(C)n2)CC1] | ¹H NMR (300 MHz, DMSO-d₆) δ 8.17 (s, 1H), 7.53 − 7.50 (m, 2H), 7.46 − 7.40 (m, 2H), 7.20 − 7.18 (m, 1H), 7.12 − 7.09 (m, 4H), 6.79 (dd, J = 16.7, 10.5 Hz, 1H), 6.26 (q, J = 7.0 Hz, 1H), 6.10 (dd, J = 16.7, 2.5 Hz, 1H), 5.67 (dd, J = 10.4, 2.5 Hz, 1H), 2.88 (dt, J = 7.9, 4.0 Hz, 1H), 2.55 (s, 3H), 1.96 (d, J = 7.0 Hz, 3H), 1.70 (s, 2H), 1.40 (s, 2H). MS: 574.25 |
| (E)-1-(4-((7-(1- (1,2,4-oxadiazol-3- yl)ethyl)-4-amino-5- (4-phenoxyphenyl)- 7H-pyrrolo[2,3- d]pyrimidin-6- yl)ethynyl)-4- hydroxypiperidin-1- yl)-4- (dimethylamino)but- 2-en-1-one | 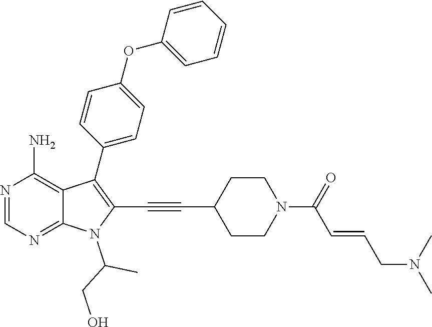 [SMILES: CC(CO)n1c(C#CC2CCN(C(=O)/C=C/CN(C)C)CC2)c(-c2ccc(Oc3ccccc3)cc2)c2c(N)ncnc21] | ¹H NMR (400 MHz, DMSO-d₆) δ 8.15 (s, 1H), 7.68 − 7.53 (m, 1H), 7.54 − 7.47 (m, 2H), 7.43 (t, J = 7.7 Hz, 2H), 7.17 (t, J = 7.4 Hz, 1H), 7.14 − 7.07 (m, 4H), 6.58 (d, J = 2.4 Hz, 2H), 6.23 (s, 1H), 5.03 (s, 1H), 4.91 (q, J = 7.0 Hz, 1H), 4.06 (q, J = 8.3, 7.2 Hz, 1H), 3.84 (dd, J = 11.1, 5.6 Hz, 1H), 3.35 (s, 2H), 3.62 (s, 2H), 3.04 − 2.98 (m, 3H), 2.14 (s, 6H), 1.79 (s, 2H), 1.53 (d, J = 7.0 Hz, 5H). MS: 579.25 |
| (E)-2-(4-amino-6- ((1-(4- (dimethylamino)but- 2-enoyl)-4- hydroxypiperidin-4- yl)ethynyl)-5-(4- phenoxyphenyl)-7H- pyrrolo[2,3- d]pyrimidin-7- yl)propanenitrile | 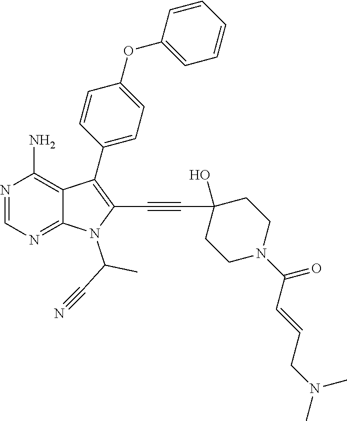 [SMILES: CC(C#N)n1c(C#CC2(O)CCN(C(=O)/C=C/CN(C)C)CC2)c(-c2ccc(Oc3ccccc3)cc2)c2c(N)ncnc21] | ¹H NMR (400 MHz, DMSO-d₆) δ 8.26 (s, 1H), 7.56 − 7.54 (m, 2H), 7.45-7.41 (t, J = 7.9 Hz, 2H), 7.20-7.16 (t, J = 7.4 Hz, 1H), 7.13- 7.10 (dd, J = 8.6, 2.7 Hz, 4H), 6.59 −6.58(d, J = 2.5 Hz, 2H), 6.18-6.13 (q, J = 7.1 Hz, 1H), 5.92 (s, 1H), 3.83-3.74 (m, 2H), 3.36(m, 1H),3.25-3.22(m, 1H),3.01-3.00 (d, J = 3.4 Hz, 2H), 2.14 (s, 6H), 1.92-1.90 (d, J = 7.2 Hz, 5H), 1.65 (s, 2H). MS: 590.20 |

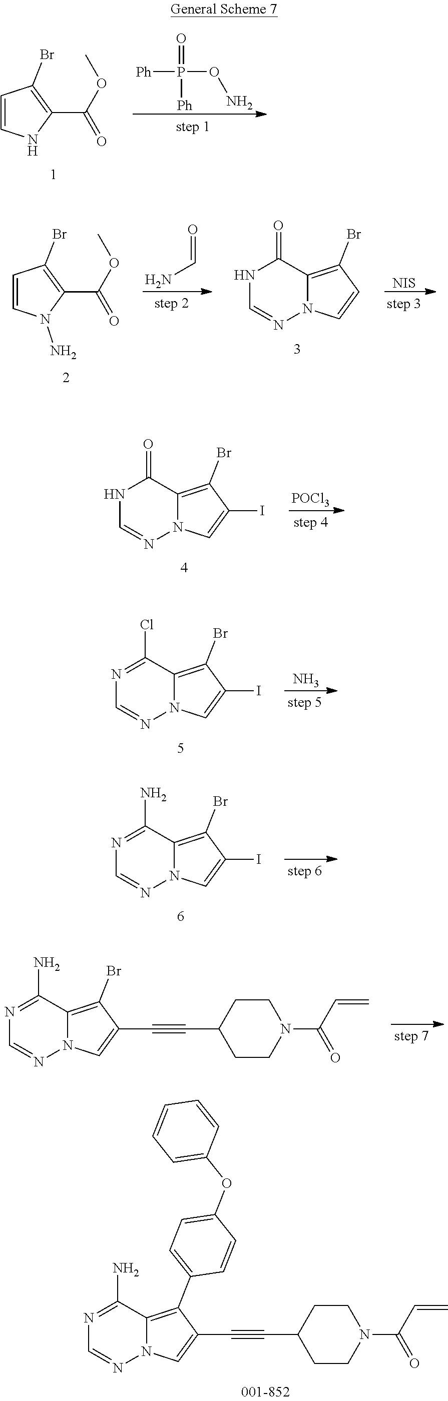

### Example 16

### 1-(4-((4-amino-5-(4-phenoxyphenyl)pyrrolo[1,2-f][1,2,4]triazin-6-yl)ethynyl)piperidin-1-yl)prop-2-en-1-one

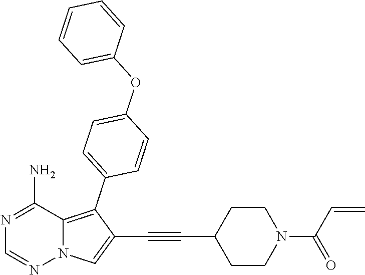 [SMILES: C=CC(=O)N1CCC(C#Cc2cn3ncnc(N)c3c2-c2ccc(Oc3ccccc3)cc2)CC1]

### methyl 1-amino-3-bromo-1H-pyrrole-2-carboxylate

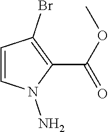

### Step 1

To a suspension of NaH (468 mg, 11.7 mmol) in dimethylformamide (1 mL) was added methyl 3-bromo-1H-pyrrole-2-carboxylate (2 g, 9.80 mmol) at 0° C. and the mixture was stirred for 30 min, followed by the addition of amino diphenylphosphinate (3.19 g, 13.7 mmol). The reaction mixture was stirred at RT for 4 h and quenched by aq. Na₂SO₃ solution. After stirred for another 5 min, the mixture was extracted with EtOAc (3*50 mL). The combined organic layers were washed with brine, dried over Na₂SO₄, filtered and concentrated to give crude product. The resulting crude material was purified by silica gel chromatography (50 g column; eluting with hexanes/ethyl acetate; ratio 10/1). Concentration in vacuo resulted in methyl 1-amino-3-bromo-1H-pyrrole-2-carboxylate (1.33 g, 62.1%) as a off-white amorphous solid.

### 5-bromopyrrolo[2,1-f][1,2,4]triazin-4(3H)-one

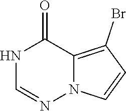 [SMILES: O=c1[nH]cnn2ccc(Br)c12]

### Step 2

A round bottomed flask was charged with methyl 1-amino-3-bromo-1H-pyrrole-2-carboxylate (1.9 g, 8.67 mmol), formamide (390 mg, 8.67 mmol) and a stirbar, and the solution was stirred at 180° C. for 3 hours. The resulting crude material was purified by silica gel chromatography (10 g column; eluting with hexanes/ethyl acetate; ratio 10/1). Concentration in vacuo resulted in 5-bromo-3H,4H-pyrrolo[2,1-f][1,2,4]triazin-4-one (855 mg, 46.2%) as a off-white amorphous solid.

### 5-bromo-6-iodopyrrolo[2,1-f][1,2,4]triazin-4(3H)-one

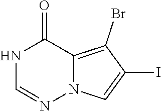 [SMILES: O=c1[nH]cnn2cc(I)c(Br)c12]

### Step 3

A round bottomed flask was charged with 5-bromo-3H,4H-pyrrolo[2,1-f][1,2,4]triazin-4-one (550 mg, 2.56 mmol), 2-iodocyclopentane-1,3-dione (687 mg, 3.07 mmol), TFA (21.2 mg, 0.18 mmol) and a stirbar. DMF (1.5 mL) was added, and the solution was stirred at 80° C. for 3 h. The resulting crude material was purified by silica gel chromatography (5 g column; eluting with hexanes/ethyl acetate; ratio 10/1). Concentration in vacuo resulted in 5-bromo-6-iodo-3H, 4H-pyrrolo[2,1-f][1,2,4]triazin-4-one (693 mg, 79.6%) as a white amorphous solid.

### 5-bromo-4-chloro-6-iodopyrrolo[2,1-f][1,2,4]triazine

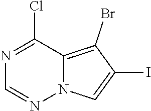 [SMILES: Clc1ncnn2cc(I)c(Br)c12]

### Step 4

A round bottomed flask was charged with 5-bromo-6-iodo-3H,4H-pyrrolo[2,1-f][1,2,4]triazin-4-one (770 mg, 2.26 mmol) and a stirbar. POCl₃(2 mL) was added, and the solution was stirred at 90° C. for 2 h. The resulting crude material was purified by silica gel chromatography (5 g column; eluting with hexanes/ethyl acetate; ratio 50/1). Concentration in vacuo resulted in 5-bromo-4-chloro-6-iodopyrrolo[2,1-f][1,2,4]triazine (390 mg, 42.8%) as a off-white amorphous solid.

### 5-bromo-6-iodopyrrolo[2,1-f][1,2,4]triazin-4-amine

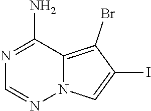 [SMILES: Nc1ncnn2cc(I)c(Br)c12]

### Step 5

A round bottomed flask was charged with 5-bromo-4-chloro-6-iodopyrrolo[2,1-f][1,2,4]triazine (360 mg, 1.00 mmol) and a stirbar. i-PrOH/NH₄OH (2 mL) was added, and the solution was stirred at 80° C. for 3 h. The resulting crude material was purified by silica gel chromatography (5 g column; eluting with hexanes/ethyl acetate; ratio 10/1). Concentration in vacuo resulted in 5-bromo-6-iodopyrrolo[2,1-f][1,2,4]triazin-4-amine (191 mg, 56.5%) as a white amorphous solid.

### 1-(4-((4-amino-5-bromopyrrolo[2,1-f][1,2,4]triazin-6-yl)ethynyl)piperidin-1-yl)prop-2-en-1-one

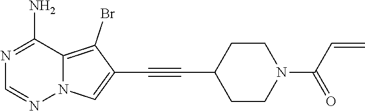 [SMILES: C=CC(=O)N1CCC(C#Cc2cn3ncnc(N)c3c2Br)CC1]

### Step 6

A round bottomed flask was charged with 5-bromo-6-iodopyrrolo[2,1-f][1,2,4]triazin-4-amine (220 mg, 0.6490 mmol), 1-(4-ethynylpiperidin-1-yl)prop-2-en-1-one (210 mg, 1.29 mmol), Pd(PPh₃)₂Cl₂ (136 mg, 0.19 mmol), CuI (73.9 mg, 0.39 mmol) and a stir-bar under N₂. THF/TEA (1.5 mL) was added, and the solution was stirred at 25° C. for overnight. The resulting crude material was purified by silica gel chromatography (5 g column; eluting with hexanes/ethyl acetate; ratio 10/1). Concentration in vacuo resulted in 1-[4-(2-{4-amino-5-bromopyrrolo[2,1-f][1,2,4]triazin-6-yl}ethynyl)piperidin-1-yl]prop-2-en-1-one (100 mg, 41.3%) as a off-white amorphous solid.

### 1-(4-((4-amino-5-(4-phenoxyphenyl)pyrrolo[2,1-f][1,2,4]triazin-6-yl)ethynyl)piperidin-1-yl)prop-2-en-1-one

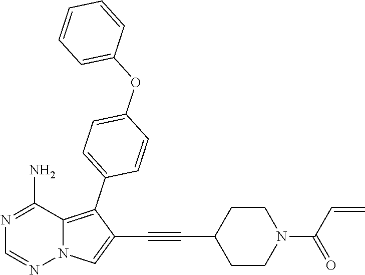 [SMILES: C=CC(=O)N1CCC(C#Cc2cn3ncnc(N)c3c2-c2ccc(Oc3ccccc3)cc2)CC1]

### Step 7

A round bottomed flask was charged with 1-[4-(2-{4-amino-5-bromopyrrolo[2,1-f][1,2,4]triazin-6-yl}ethynyl)piperidin-1-yl]prop-2-en-1-one (240 mg, 0.6412 mmol), (4-phenoxyphenyl)boronic acid (164 mg, 0.7694 mmol), Pd(DtBPF)Cl₂ (41.7 mg, 0.06412 mmol), K₂CO₃ (264 mg, 1.92 mmol) and a stirbar. 1,4-dioxane/water (2.5 mL) was added, and the solution was stirred at 80° C. for overnight. The resulting crude material was purified by HPLC (Column: XBridge Prep C₁₈ OBD Column 19×150 mm 5 um; Mobile Phase A:Water (10 MMOL/L NH₄HCO₃), Mobile Phase B: ACN; Flow rate: 20 mL/min; Gradient: 40% B to 56% B in 12 min; 254&220 nm; Rt: 8.18 11.25 min.). Lyophilization yielded 1-(4-{2-[4-amino-5-(4-phenoxyphenyl)pyrrolo[2,1-f][1,2,4]triazin-6-yl]ethynyl}piperidin-1-yl)prop-2-en-1-one (30 mg, 20%) as a white amorphous solid.

Characterization data for the product are listed in Table 11, below.

TABLE 11
Characterization Data

| | | Proton NMR & |
|---|---|---|
| Compound | Structure | Mass Spec. (obs.) |
| 1-(4-((4-amino- 5-(4- phenoxyphenyl) pyrrolo[2,1- f][1,2,4]triazin-6- yl)ethynyl)piperidin- 1-yl)prop-2-en-1- one | 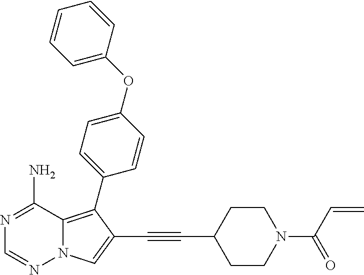 [SMILES: C=CC(=O)N1CCC(C#Cc2cn3ncnc(N)c3c2-c2ccc(Oc3ccccc3)cc2)CC1] | ¹H NMR (400 MHz, DMSO-d₆) δ 7.92 (d, J = 15.3 Hz, 2H), 7.53 − 7.45 (m, 2H), 7.43 − 7.40 (m, 1H), 7.20 − 7.14 (m, 1H), 7.14 − 7.04 (m, 4H), 6.77 (dd, J = 16.7, 10.5 Hz, 1H), 6.08 (dd, J = 16.7, 2.4 Hz, 1H), 5.66 (dd, J = 10.4, 2.4 Hz, 1H), 3.54 (s, 2H), 3.43 (s, 2H), 2.95 − 2.77 (m, 1H), 1.72 (s, 2H), 1.57 − 1.40 (m, 2H). MS: 464.6 |

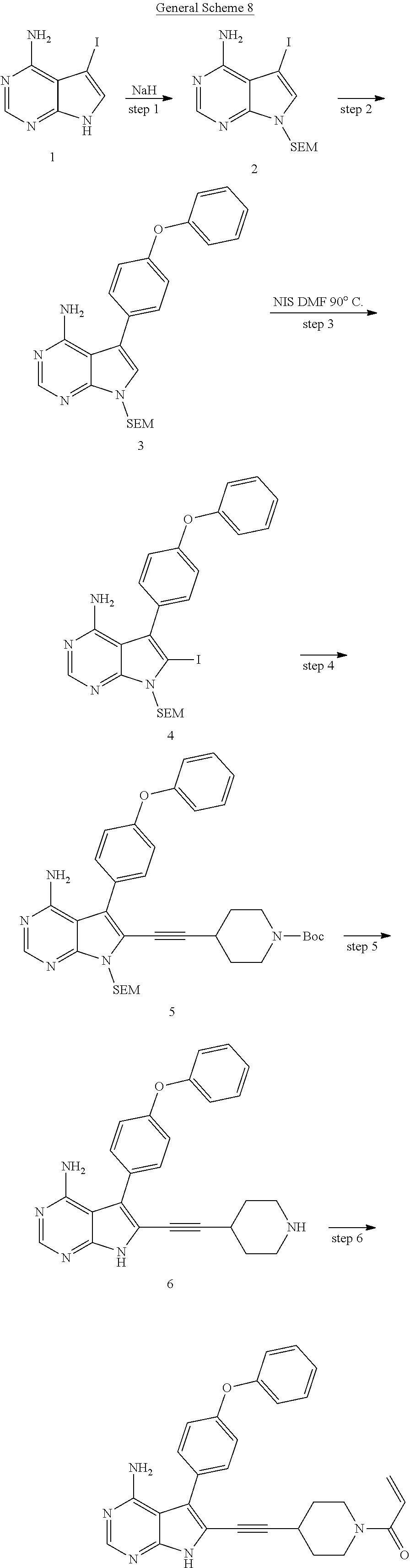

### Example 17

### 1-(4-((4-amino-5-(4-phenoxyphenyl)-7H-pyrrolo[2,3-d]pyrimidin-6-yl)ethynyl)piperidin-1-yl)prop-2-en-1-one

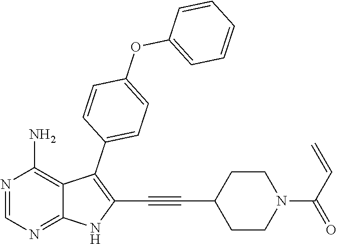 [SMILES: C=CC(=O)N1CCC(C#Cc2[nH]c3ncnc(N)c3c2-c2ccc(Oc3ccccc3)cc2)CC1]

### 5-iodo-7-((2-(trimethylsilyl)ethoxy)methyl)-7H-pyrrolo[2,3-d]pyrimidin-4-amine

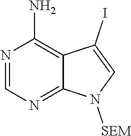 [SMILES: C[Si](C)(C)CCOCn1cc(I)c2c(N)ncnc21]

### Step 1

5-iodo-7H-pyrrolo[2,3-d]pyrimidin-4-amine (800 mg, 3.07 mmol) was dissolved anhydrous DMF (20 ml) under N₂. NaH (60% suspension in mineral oil; 146.7 mg, 3.68 mmol) was added at 0° C. The reaction mixture was stirred at 0° C. for 30 min. SEMCl (613 mg, 3.68 mmol) was added dropwise at 0° C. and stirring was continued at 0° C. for 1 hour. Then the reaction was quenched with water (100 mL) at 0° C. and separated with EtOAc (100 mL×3). The organic layers were dried over Na₂SO₄, filtered and concentrated in vacuo. The crude product was purified by silica gel chromatography (DCM/MeOH: 100:0˜60:1) to give 5-iodo-7H-pyrrolo[2,3-d]pyrimidin-4-amine (654 mg, yield: 54.5%) as a off-white amorphous solid.

### 5-(4-phenoxyphenyl)-7-((2-(trimethylsilyl)ethoxy)methyl)-7H-pyrrolo[2,3-d]pyrimidin-4-amine

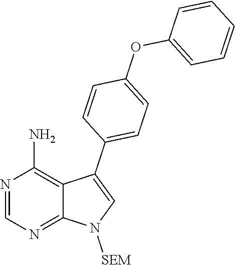 [SMILES: C[Si](C)(C)CCOCn1cc(-c2ccc(Oc3ccccc3)cc2)c2c(N)ncnc21]

### Step 2

A resealable reaction vial was charged with 5-iodo-7H-pyrrolo[2,3-d]pyrimidin-4-amine (640 mg, 1.64 mmol), (4-phenoxyphenyl)boronic acid (383 mg, 1.79 mmol), Pd(DtBPF)Cl₂ (105 mg, 163 μmol), K₂CO₃ (684 mg, 4.89 mmol), and a stirbar before being evacuated and purged with nitrogen three times. Dioxane (12 mL) and H₂O (3 mL) was added, and the mixture was stirred for 1 h at 90° C. The reaction mixture was diluted with H₂O (50 mL), and the aqueous phase was extracted with ethyl acetate (800 mL) three times. The combined organic layers were washed with brine, dried over sodium sulfate, filtered, and concentrated in vacuo. The resulting crude material was purified by silica gel chromatography (eluting with PE/EA=1/1). Concentration in vacuo resulted in 5-(4-phenoxyphenyl)-7-((2-(trimethylsilyl)ethoxy)methyl)-7H-pyrrolo[2,3-d]pyrimidin-4-amine (635 mg, 90.0% yield) as an orange oil.

### 6-iodo-5-(4-phenoxyphenyl)-7-((2-(trimethylsilyl)ethoxy)methyl)-7H-pyrrolo[2,3-d]pyrimidin-4-amine

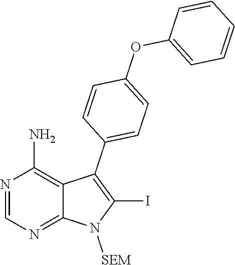 [SMILES: C[Si](C)(C)CCOCn1c(I)c(-c2ccc(Oc3ccccc3)cc2)c2c(N)ncnc21]

### Step 3

A resealable reaction vial was charged with 5-(4-phenoxyphenyl)-7-((2-(trimethylsilyl)ethoxy)methyl)-7H-pyrrolo[2,3-d]pyrimidin-4-amine (625 mg, 1.45 mmol), 1-iodopyrrolidine-2,5-dione (386 mg, 1.72 mmol), DMF (10 mL) and a stirbar before being evacuated and purged with nitrogen three times. The mixture was stirred for 2 h at 90° C. The reaction mixture was diluted with aqueous Na₂SO₃ (10 mL), and the aqueous phase was extracted with ethyl acetate (30 mL) three times. The combined organic layers were washed with saturated brines, dried over sodium sulfate, filtered, and concentrated in vacuo. The resulting crude material was purified by silica gel chromatography (eluting with PE/EA=3/1). Concentration in vacuo resulted in 6-iodo-5-(4-phenoxyphenyl)-7-((2-(trimethylsilyl)ethoxy)methyl)-7H-pyrrolo[2,3-d]pyrimidin-4-amine (280 mg, 34.7% yield) as a yellow amorphous solid.

### 1-(4-((4-amino-5-(4-phenoxyphenyl)-7-((2-(trimethylsilyl)ethoxy)methyl)-7H-pyrrolo[2,3-d]pyrimidin-6-yl)ethynyl)piperidin-1-yl)prop-2-en-1-one

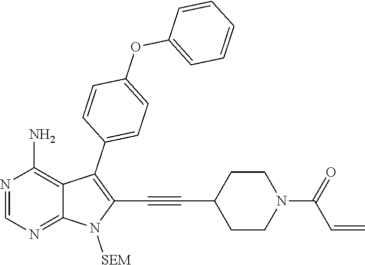 [SMILES: C=CC(=O)N1CCC(C#Cc2c(-c3ccc(Oc4ccccc4)cc3)c3c(N)ncnc3n2COCC[Si](C)(C)C)CC1]

### Step 4

A resealable reaction vial was charged with 6-iodo-5-(4-phenoxyphenyl)-7-((2-(trimethylsilyl)ethoxy)methyl)-7H-pyrrolo[2,3-d]pyrimidin-4-amine (270 mg, 484 mol), 1-(4-ethynylpiperidin-1-yl)prop-2-en-1-one (157.8 mg, 968 μmol), Pd(PPh₃)₂Cl₂ (67.5 mg, 96.8 μmol), CuI (18 mg, 96.8 μmol), TEA (185.5 mg, 1.836 mmol), DMF (7 mL) was added and a stirbar before being evacuated and purged with nitrogen three times. The mixture was stirred for 2 h at 60° C. The reaction mixture was diluted with H₂O (20 mL), and the aqueous phase was extracted with ethyl acetate (50 mL) three times. The combined organic layers were washed with saturated brines, dried over sodium sulfate, filtered, and concentrated in vacuo. The resulting crude material was purified by silica gel chromatography (DCM/MeOH: 100:0-80:1). Concentration in vacuo resulted in 1-(4-((4-amino-5-(4-phenoxyphenyl)-7-((2-(trimethylsilyl)ethoxy)methyl)-7H-pyrrolo[2,3-d]pyrimidin-6-yl)ethynyl)piperidin-1-yl)prop-2-en-1-one (79 mg, 27.5% yield) as a yellow amorphous solid.

### 1-(4-((4-amino-5-(4-phenoxyphenyl)-7H-pyrrolo[2,3-d]pyrimidin-6-yl)ethynyl)piperidin-1-yl)prop-2-en-1-one

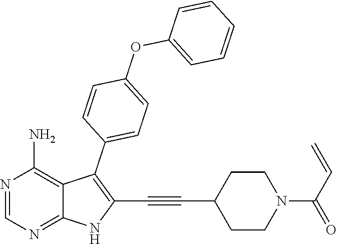 [SMILES: C=CC(=O)N1CCC(C#Cc2[nH]c3ncnc(N)c3c2-c2ccc(Oc3ccccc3)cc2)CC1]

### Step 5

A round bottomed flask was charged with 1-(4-((4-amino-5-(4-phenoxyphenyl)-7-((2-(trimethylsilyl)ethoxy)methyl)-7H-pyrrolo[2,3-d]pyrimidin-6-yl)ethynyl)piperidin-1-yl)prop-2-en-1-one (79 mg, 133 μmol) and a stirbar. DCM (2 mL) and TFA (0.5 mL) was added, and the solution was stirred for 1 h at room temperature. LCMS monitoring was done to check complete conversion to intermediate hydroxylmethyl derivative. After completion of reaction, the solvents were evaporated in vacuo and azeotroped with toluene to remove trance TFA, then dissolved in MeOH (4 ml) and ethylenediamine (0.1 ml) was added. The mixture reaction was stirred for 0.5 hour at rt before the mixture was purified by flash chromatography (Flash Column: Spec: C18, 20 g; Mobile Phase A:Water (0.5% NH₄HCO₃), Mobile Phase B: ACN; Flow rate: 60 mL/min; 254 nm) to give the crude product. Then the residue was purified by HPLC (Column: XBridge Shield RP18 OBD Column, 5 um, 19*150 mm; Mobile Phase A:Water (10 MMOL/L NH₄HCO₃), Mobile Phase B: ACN; Flow rate: 20 mL/min; Gradient: 30% B to 52% B in 8 min; 254 nm; Rt: 8.15 min) to give 1-(4-((4-amino-5-(4-phenoxyphenyl)-7H-pyrrolo[2,3-d]pyrimidin-6-yl)ethynyl)piperidin-1-yl)prop-2-en-1-one (22.6 mg, 36.6% yield) as a off-white amorphous solid.

Characterization data for the product are listed in Table 12, below.

TABLE 12
Characterization Data

| | | Proton NMR & |
|---|---|---|
| Compound | Structure | Mass Spec. (obs.) |
| 1-(4-((4-amino-5-(4- phenoxyphenyl)-7H- pyrrolo[2,3- d]pyrimidin-6- yl)ethynyl)piperidin- 1-yl)prop-2-en-1-one | 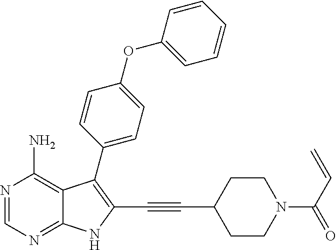 [SMILES: C=CC(=O)N1CCC(C#Cc2[nH]c3ncnc(N)c3c2-c2ccc(Oc3ccccc3)cc2)CC1] | ¹H NMR (300 MHz, DMSO-d₆) δ 8.13 (s, 1H), 7.56 − 7.37 (m, 4H), 7.23 − 7.05 (m, 5H), 6.79 (dd, J = 16.7, 10.5 Hz, 1H), 6.09 (dd, J = 16.6, 2.4 Hz, 1H), 5.66 (dd, J = 10.4, 2.4 Hz, 1H), 3.62 (m, 2H), 3.48 (m,2H), 2.99 (m, 1H), 1.77 (m, 2H), 1.54 (m, 2H). MS: 464.2 |

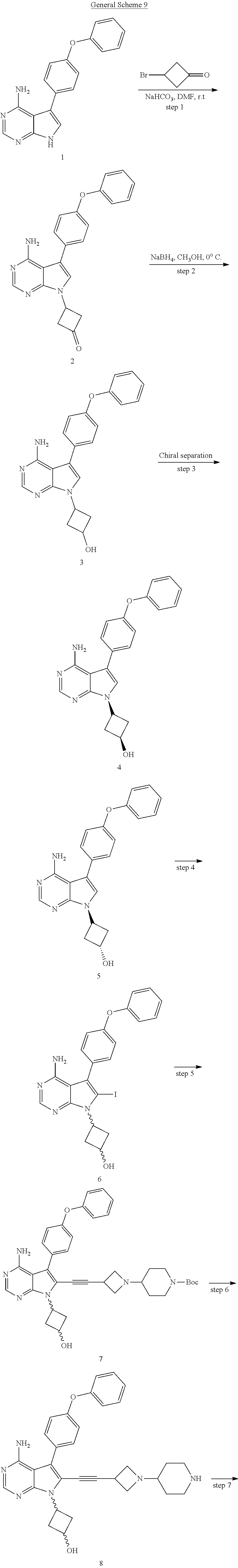

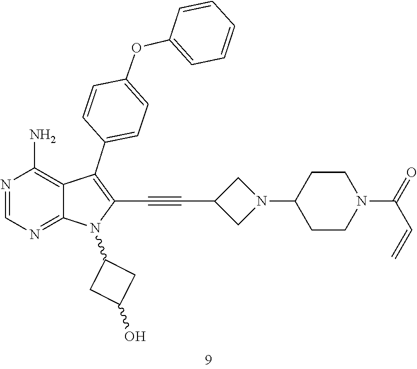 [SMILES: C=CC(=O)N1CCC(N2CC(C#Cc3c(-c4ccc(Oc5ccccc5)cc4)c4c(N)ncnc4n3C3CC(O)C3)C2)CC1]

### Example 18

### 1-(4-((4-amino-7-isopropyl-5-(4-phenoxyphenyl)-7H-pyrrolo[2,3-d]pyrimidin-6-yl)ethynyl)piperidin-1-yl)prop-2-en-1-one

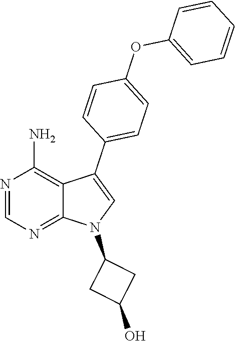 [SMILES: Nc1ncnc2c1c(-c1ccc(Oc3ccccc3)cc1)cn2[C@H]1C[C@@H](O)C1]

### 3-(4-amino-5-(4-phenoxyphenyl)-7H-pyrrolo[2,3-d]pyrimidin-7-yl)cyclobutanone

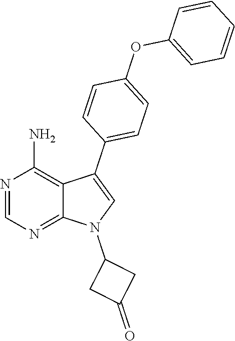 [SMILES: Nc1ncnc2c1c(-c1ccc(Oc3ccccc3)cc1)cn2C1CC(=O)C1]

### Step 1

A round bottomed flask was charged with 5-(4-phenoxyphenyl)-7H-pyrrolo[2,3-d]pyrimidin-4-amine (800 mg, 2.64 mmol), 3-bromocyclobutane-1-one (1.17 g, 7.92 mmol), NaHCO₃ (1.10 g, 13.2 mmol) and a stirbar. DMF (15 mL) was added, and the solution was stirred at 30° C. for 5 hours. The resulting crude material was purified by FLASH (acetonitrile/water). Concentration in vacuo, obtained 3-[4-amino-5-(4-phenoxyphenyl)-7H-pyrrolo[2,3-d]pyrimidin-7-yl]cyclobutan-1-one (650 mg, 66.5%) as a yellow solid.

### 3-(4-amino-5-(4-phenoxyphenyl)-7H-pyrrolo[2,3-d]pyrimidin-7-yl)cyclobutan-1-ol

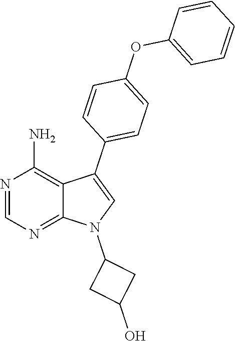 [SMILES: Nc1ncnc2c1c(-c1ccc(Oc3ccccc3)cc1)cn2C1CC(O)C1]

### Step 2

A round bottomed flask was charged with 3-[4-amino-5-(4-phenoxyphenyl)-7H-pyrrolo[2,3-d]pyrimidin-7-yl]cyclobutan-1-one (650 mg, 1.75 mmol), CH₃OH (20 mL) and a stirbar. NaBH₄ (166 mg, 4.37 mmol) was added, and the solution was stirred at 0° C. for 1.5 hours. The reaction mixture was poured into 150 mL of water, then extracted with CH₂Cl₂ (3×150 mL). The combined organic layer was dried over anhydrous sodium sulfate and concentrated under vacuum to give (650 mg, 93.7%) product as a yellow solid.

### (1s,3s)-3-(4-amino-5-(4-phenoxyphenyl)-7H-pyrrolo[2,3-d]pyrimidin-7-yl)cyclobutanol

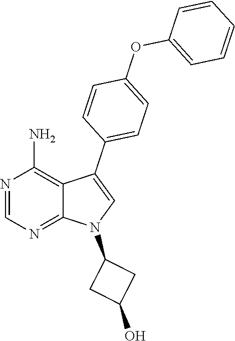 [SMILES: Nc1ncnc2c1c(-c1ccc(Oc3ccccc3)cc1)cn2[C@H]1C[C@@H](O)C1]

### (1r,3r)-3-(4-amino-5-(4-phenoxyphenyl)-7H-pyrrolo[2,3-d]pyrimidin-7-yl)cyclobutanol

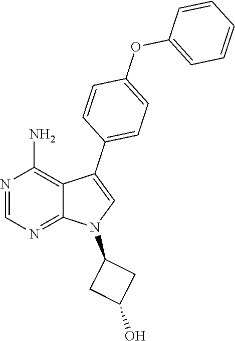 [SMILES: Nc1ncnc2c1c(-c1ccc(Oc3ccccc3)cc1)cn2[C@H]1C[C@H](O)C1]

### Step 3

The resulting crude material was purified by Chiral HPLC (Hex (0.2% IPAmine):EtOH=70:30).

Concentration in vacuo resulted in (1s,3s)-3-(4-amino-5-(4-phenoxyphenyl)-7H-pyrrolo[2,3-d]pyrimidin-7-yl)cyclobutanol (400 mg, 66.7%1) as a off-white amorphous solid.

Concentration in vacuo resulted in (1r,3r)-3-[4-amino-5-(4-phenoxyphenyl)-7H-pyrrolo[2,3-d]pyrimidin-7-yl]cyclobutan-1-ol (70.0 mg, 11.6%) as a off-white amorphous solid.

### (1s,3s)-3-(4-amino-6-iodo-5-(4-phenoxyphenyl)-7H-pyrrolo[2,3-d]pyrimidin-7-yl)cyclobutan-1-ol

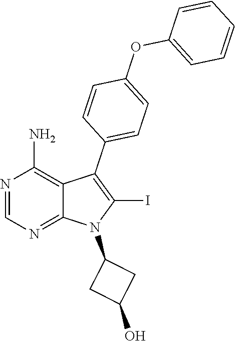 [SMILES: Nc1ncnc2c1c(-c1ccc(Oc3ccccc3)cc1)c(I)n2[C@H]1C[C@@H](O)C1]

### Step 4

A round bottomed flask was charged with 3-[4-amino-5-(4-phenoxyphenyl)-7H-pyrrolo[2,3-d]pyrimidin-7-yl]cyclobutan-1-ol (400 mg, 1.07 mmol), NIS (240 mg, 1.07 mmol), CF₃COOH (365 mg, 3.21 mmol) and a stirbar. CH₂Cl₂ (40 mL) was added, and the solution was stirred at 25° C. for 1.5 hours. The pH value of the solution was adjusted to 6-7 with NaHCO₃. The resulting solution was diluted with 100 mL of water, then extracted with CH₂Cl₂ (3×100 mL). The organic layers were combined, washed with sodium carbonate (aq.) and brine, dried and con centration in vacuo resulted in 3-[4-amino-6-iodo-5-(4-phenoxyphenyl)-7H-pyrrolo[2,3-d]pyrimidin-7-yl]cyclobutan-1-ol (350 mg, 65.6%) as a off-white amorphous solid.

### tert-butyl 4-(3-((4-amino-7-((1r,3r)-3-hydroxycyclobutyl)-5-(4-phenoxyphenyl)-7H-pyrrolo[2,3-d]pyrimidin-6-yl)ethynyl)azetidin-1-yl)piperidine-1-carboxylate

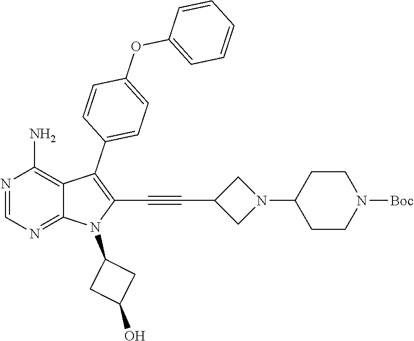 [SMILES: CC(C)(C)OC(=O)N1CCC(N2CC(C#Cc3c(-c4ccc(Oc5ccccc5)cc4)c4c(N)ncnc4n3[C@H]3C[C@@H](O)C3)C2)CC1]

### Step 5

A round bottomed flask was charged with 3-[4-amino-6-iodo-5-(4-phenoxyphenyl)-7H-pyrrolo[2,3-d]pyrimidin-7-yl]cyclobutan-1-ol (250 mg, 501 μmol), tert-butyl 4-(3-ethynylazetidin-1-yl)piperidine-1-carboxylate (264 mg, 1.00 mmol), CuI (56.9 mg, 300 μmol), Pd(PPh₃)₂Cl₂ (105 mg, 150 μmol), TEA (151 mg, 1.50 mmol) and a stirbar. THE (12 mL) was added, and the solution was stirred at 60° C. for 3 hours. The resulting crude material was purified by FLASH (acetonitrile/water), and concentrated in vacuo. tert-butyl 4-(3-{2-[4-amino-7-(3-hydroxycyclobutyl)-5-(4-phenoxyphenyl)-7H-pyrrolo[2,3-d]pyrimidin-6-yl]ethynyl}azetidin-1-yl)piperidine-1-carboxylate (125 mg, 39.4%) as a off-white amorphous solid.

### (1s,3s)-3-(4-amino-5-(4-phenoxyphenyl)-6-((1-(piperidin-4-yl)azetidin-3-yl)ethynyl)-7H-pyrrolo[2,3-d]pyrimidin-7-yl)cyclobutan-1-ol

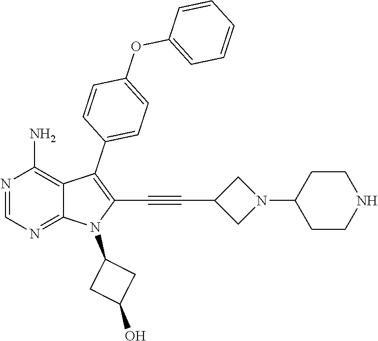 [SMILES: Nc1ncnc2c1c(-c1ccc(Oc3ccccc3)cc1)c(C#CC1CN(C3CCNCC3)C1)n2[C@H]1C[C@@H](O)C1]

### Step 6

A round bottomed flask was charged with tert-butyl 4-(3-{2-[4-amino-7-(3-hydroxycyclobutyl)-5-(4-phenoxyphenyl)-7H-pyrrolo[2,3-d]pyrimidin-6-yl]ethynyl}azetidin-1-yl)piperidine-1-carboxylate (125 mg, 196 μmol), and a stirbar. CH₂Cl₂ (8 mL) and CF₃COOH (2 mL) was added, and the solution was stirred at 25° C. for 30 minutes. The resulting reaction mixture was concentrated under vacuum to give 3-[4-amino-5-(4-phenoxyphenyl)-6-{2-[1-(piperidin-4-yl)azetidin-3-yl]ethynyl}-7H-pyrrolo[2,3-d]pyrimidin-7-yl]cyclobutan-1-ol (100 mg, 96.1%) as a off-white amorphous solid.

### 1-(4-(3-((4-amino-7-((1r,3r)-3-hydroxycyclobutyl)-5-(4-phenoxyphenyl)-7H-pyrrolo[2,3-d]pyrimidin-6-yl)ethynyl)azetidin-1-yl)piperidin-1-yl)prop-2-en-1-one

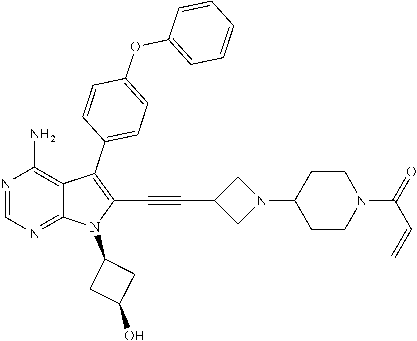 [SMILES: C=CC(=O)N1CCC(N2CC(C#Cc3c(-c4ccc(Oc5ccccc5)cc4)c4c(N)ncnc4n3[C@H]3C[C@@H](O)C3)C2)CC1]

### Step 7

A round bottomed flask was charged with 3-[4-amino-5-(4-phenoxyphenyl)-6-{2-[1-(piperidin-4-yl)azetidin-3-yl]ethynyl}-7H-pyrrolo[2,3-d]pyrimidin-7-yl]cyclobutan-1-ol (100 mg, 6.63 μmol), CH2Cl2 (8 mL), TEA (1.99 mg, 19.8 μmol) and a stirbar. prop-2-enoyl chloride (539 μg, 5.96 μmol) was added slowly, and the solution was stirred at 0° C. for 0.5 hour. The reaction mixture was poured into 10 mL of water, then extracted with CH₂Cl₂ (3×10 mL). The organic layers were combined, washed with sodium carbonate (aq.) and brine, dried and concentrated under vacuum.

The resulting crude material was purified by HPLC Column: XBridge Prep C18 OBD Column, 5 um, 19*150 mm; Mobile Phase A:Water (10 MMOL/L NH4HCO3), Mobile Phase B: ACN; Flow rate: 20 mL/min; Gradient: 30% B to 70% B in 7 min; 254&220 nm; Rt: 5.6 min. Lyophilization yielded 1-[4-(3-{2-[4-amino-7-(3-hydroxycyclobutyl)-5-(4-phenoxyphenyl)-7H-pyrrolo[2,3-d]pyrimidin-6-yl]ethynyl}azetidin-1-yl)piperidin-1-yl]prop-2-en-1-one (35.5 mg, 32.2%) as a off-white amorphous solid.

Compounds prepared by substantially the same methods, and their characterization data, are listed in Table 13, below.

TABLE 13
Selected Compounds

| | | Proton NMR & |
|---|---|---|
| Compound | Structure | Mass Spec. (obs.) |
| 1-(4-(3-((4-amino-7-((1r,3r)- 3-hydroxycyclobutyl)-5-(4- phenoxyphenyl)-7H- pyrrolo[2,3-d]pyrimidin-6- yl)ethynyl)azetidin-1- yl)piperidin-1-yl)prop-2-en- 1-one | 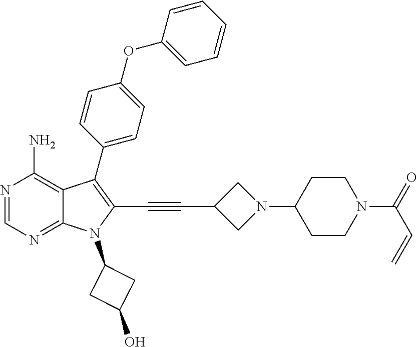 [SMILES: C=CC(=O)N1CCC(N2CC(C#Cc3c(-c4ccc(Oc5ccccc5)cc4)c4c(N)ncnc4n3[C@H]3C[C@@H](O)C3)C2)CC1] | ¹H NMR (400 MHz, DMSO-d₆) δ 8.18 (s, 1H), 7.52 − 7.50 (d, J = 8.2 Hz, 2H), 7.45 − 7.42 (t, J = 7.6 Hz, 2H), 7.21- 7.12 (m, J = 28.4, 7.8 Hz, 5H), 6.82 − 6.75 (dd, J = 16.7, 10.5 Hz, 1H), 6.127- 6.05 (m, 1H), 5.70 − 5.64 (m, 1H), 5.41 (d, J = 6.0 Hz, 1H), 4.87 − 4.81 (t, J = 8.8 Hz, 1H), 4.08 − 3.98 (m, 2H), 3.85 − 3.82 (d, J = 13.2 Hz, 1H), 3.53 − 3.49 (t, J = 7.0 Hz, 2H), 3.37 − 3.43 (t, J = 7.3 Hz, 1H), 3.13 − 3.06 (m, 5H), 2.95 − 2.90 (t, J = 11.3 Hz, 1H), 2.70 (d, J = 9.2 Hz, 2H), 2.27 (s, 1H), 1.59 (s, 2H), 1.05 (s, 2H). MS: 589.25 |
| 1-(4-(3-((4-amino-7-((1s,3s)- 3-hydroxycyclobutyl)-5-(4- phenoxyphenyl)-7H- pyrrolo[2,3-d]pyrimidin-6- yl)ethynyl)azetidin-1- yl)piperidin-1-yl)prop-2-en- 1-one | 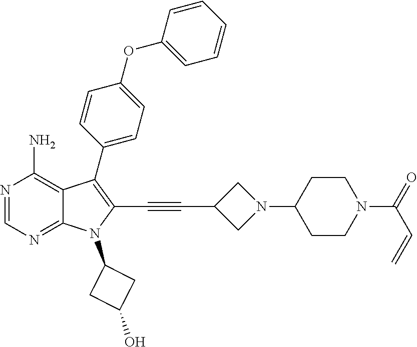 [SMILES: C=CC(=O)N1CCC(N2CC(C#Cc3c(-c4ccc(Oc5ccccc5)cc4)c4c(N)ncnc4n3[C@H]3C[C@H](O)C3)C2)CC1] | ¹H NMR (400 MHz, Chloroform-d) δ 8.35 (s, 1H), 7.53 − 7.51 (d, J = 8.3 Hz, 2H), 7.43 − 7.39 (t, J = 7.8 Hz, 2H), 7.28 − 7.19 (t, J = 7.4 Hz, 1H), 7.17 − 7.10 (t, 4H), 6.62 −6.55 (m, J = 16.8, 10.6 Hz, 1H), 6.29 − 6.25 (dd, J = 16.8, 1.9 Hz, 2H), 5.74 − 5.67 (m, 2H), 5.19 (s, 2H), 4.88 − 4.85 (t, J = 7.1 Hz, 1H), 4.26 (s, 1H), 3.89 −3.83 (s, 1H), 3.71 − 3.67 (t, J = 7.0 Hz, 2H), 3.56 − 3.45 (m, J = 14.5, 7.1 Hz, 3H), 3.20 (s, 1H), 3.19 − 3.08 (t, J = 7.2 Hz, 3H), 2.57 − 2.52 (t, J = 11.1 Hz, 2H), 2.33 − 2.31 (d, J = 9.5 Hz, 1H), 1.73 −1.70 (d, J = 13.1 Hz, 2H), 1.34 −1.28 (d, J = 13.0 Hz, 2H). MS: 589.25 |

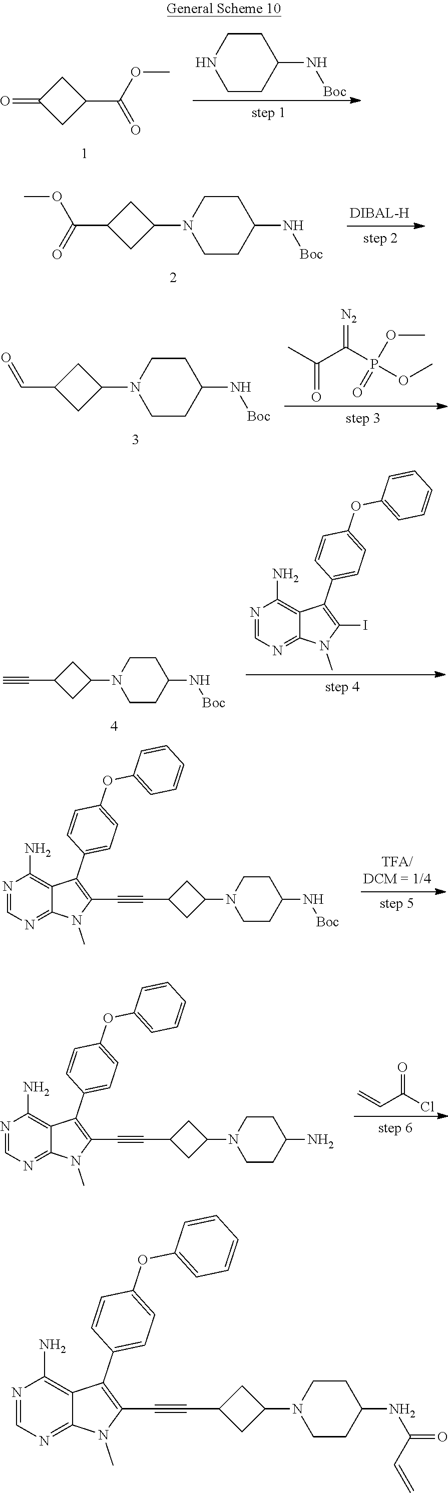

### Example 19

### N-(1-(3-((4-amino-7-methyl-5-(4-phenoxyphenyl)-7H-pyrrolo[2,3-d]pyrimidin-6-yl)ethynyl)cyclobutyl)piperidin-4-yl)acrylamide

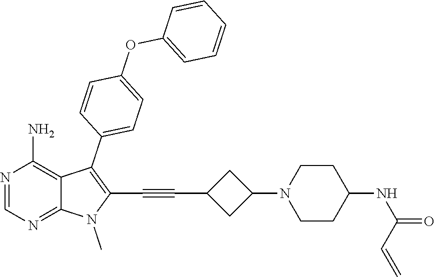 [SMILES: C=CC(=O)NC1CCN(C2CC(C#Cc3c(-c4ccc(Oc5ccccc5)cc4)c4c(N)ncnc4n3C)C2)CC1]

### methyl-3-(4-(tert-butoxycarbonylamino)piperidin-1-yl)cyclobutanecarboxylate

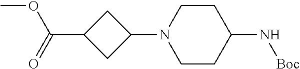 [SMILES: COC(=O)C1CC(N2CCC(NC(=O)OC(C)(C)C)CC2)C1]

### Step 1

A round bottomed flask was charged with methyl 3-oxocyclobutane-1-carboxylate (7.66 g, 59.8 mmol), tert-butyl N-(piperidin-4-yl)carbamate (8 g, 39.9 mmol), DCM (100 mL), DIEA (15.3 g, 119 mmol) and a stirbar. The mixture was stirred and cooled to 0° C., NaBH(OAc)₃ (16.9 g, 79.8 mmol) was added, and the solution was stirred for 3 h at room temperature. The reaction mixture was diluted with H₂O (100 mL), and the aqueous phase was extracted with DCM (200 mL) three times. The combined organic layers were washed with brines, dried over sodium sulfate, filtered, and concentrated in vacuo. The resulting crude material was purified by silica gel chromatography (eluting with EA/PE=1/4). Concentration in vacuo resulted in methyl 3-(4-{[(tert-butoxy)carbonyl]amino}piperidin-1-yl)cyclobutane-1-carboxylate (9.00 g, 72.5%) as a white amorphous solid.

### tert-butyl-1-(3-formylcyclobutyl)piperidin-4-ylcarbamate

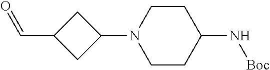 [SMILES: CC(C)(C)OC(=O)NC1CCN(C2CC(C=O)C2)CC1]

### Step 2

A resealable reaction vial was charged with methyl 3-(4-{[(tert-butoxy)carbonyl]amino}piperidin-1-yl)cyclobutane-1-carboxylate (3.5 g, 11.2 mmol), DCM (40 mL), and a stirbar before being evacuated and purged with nitrogen three times. The mixture was stirred and cooled to −78° C., then DIBAL-H (13.5 mL, 1 mol/mL dissolved in toluene) was added and the mixture was stirred for 4 h at −78° C. The reaction mixture was diluted with H₂O (10 mL), and the aqueous phase was extracted with DCM (50 mL) three times. The combined organic layers were washed with brines, dried over sodium sulfate, filtered, and concentrated in vacuo. The resulting crude material was purified by silica gel chromatography (eluting with EA/PE=1/2). Concentration in vacuo resulted in tert-butyl N-[1-(3-formylcyclobutyl)piperidin-4-yl]carbamate (2.50 g, 79.1%) as a yellow amorphous solid.

### tert-butyl-1-(3-ethylcyclobutyl)piperidin-4-ylcarbamate

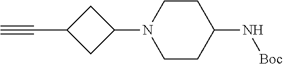 [SMILES: C#CC1CC(N2CCC(NC(=O)OC(C)(C)C)CC2)C1]

### Step 3

A round bottomed flask was charged with tert-butyl N-[1-(3-formylcyclobutyl)piperidin-4-yl]carbamate (2.45 g, 8.67 mmol), K₂CO₃ (1.19 g, 8.67 mmol), MeOH (30 mL) and a stirbar. The mixture was stirred and cooled to 0° C., dimethyl (1-diazo-2-oxopropyl)phosphonate (2.49 g, 13.0 mmol) was added, and the solution was stirred for 4 h at room temperature. The reaction mixture was diluted with H₂O (50 mL), and the aqueous phase was extracted with EA (150 mL) three times. The combined organic layers were washed with brines, dried over sodium sulfate, filtered, and concentrated in vacuo. The resulting crude material was purified by silica gel chromatography (eluting with EA/PE=1/5). Concentration in vacuo resulted in tert-butyl N-[1-(3-ethynylcyclobutyl)piperidin-4-yl]carbamate (1.80 g, 74.6%) as a yellow amorphous solid.

### tert-butyl-1-(3-((4-amino-7-methyl-5-(4-phenoxyphenyl)-7H-pyrrolo[2,3-d]pyrimidin-6-yl)ethynyl)cyclobutyl)piperidin-4-ylcarbamate

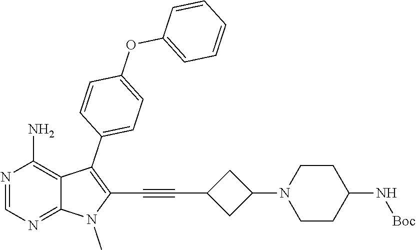 [SMILES: Cn1c(C#CC2CC(N3CCC(NC(=O)OC(C)(C)C)CC3)C2)c(-c2ccc(Oc3ccccc3)cc2)c2c(N)ncnc21]

### Step 4

A resealable reaction vial was charged with 6-iodo-7-methyl-5-(4-phenoxyphenyl)-7H-pyrrolo[2,3-d]pyrimidin-4-amine (700 mg, 1.58 mmol), tert-butyl N-[1-(3-ethynylcyclobutyl)piperidin-4-yl]carbamate (659 mg, 2.37 mmol), Pd(PPh₃)₂Cl₂ (221 mg, 316 μmol), CuI (120 mg, 632 μmol), DMF (10 mL), TEA (1.59 g, 15.8 mmol) and a stirbar before being evacuated and purged with nitrogen three times. The mixture was stirred for 2 h at 60° C. The reaction mixture was diluted with H₂O (50 mL), and the aqueous phase was extracted with EA (150 mL) three times. The combined organic layers were washed with brines, dried over sodium sulfate, filtered, and concentrated in vacuo. The resulting crude material was purified by silica gel chromatography (eluting with EA/MeOH=50/1). Concentration in vacuo resulted in tert-butyl N-[1-(3-{2-[4-amino-7-methyl-5-(4-phenoxyphenyl)-7H-pyrrolo[2,3-d]pyrimidin-6-yl]ethynyl}cyclobutyl)piperidin-4-yl]carbamate (700 mg, 74.7%) as a yellow amorphous solid.

### 6-((3-(4-aminopiperidin-1-yl)cyclobutyl)ethynyl)-7-methyl-5-(4-phenoxyphenyl)-7H-pyrrolo[2,3-d]pyrimidin-4-amine

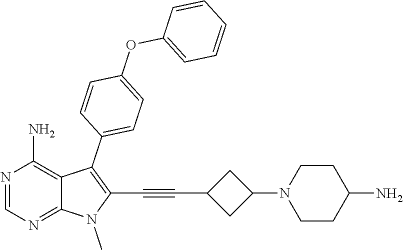 [SMILES: Cn1c(C#CC2CC(N3CCC(N)CC3)C2)c(-c2ccc(Oc3ccccc3)cc2)c2c(N)ncnc21]

### Step 5

A round bottomed flask was charged with tert-butyl N-[1-(3-{2-[4-amino-7-methyl-5-(4-phenoxyphenyl)-7H-pyrrolo[2,3-d]pyrimidin-6-yl]ethynyl}cyclobutyl)piperidin-4-yl]carbamate (690 mg, 1.16 mmol), DCM (6 mL), TFA (1.5 mL) and a stirbar. The solution was stirred for 1 h at room temperature. The reaction mixture was diluted with H₂O (20 mL), and the pH of the solution was adjusted to 9-10 with Na₂CO₃ saturated solution. Then the aqueous phase was extracted with EA (300 mL) three times. The combined organic layers were washed with brines, dried over sodium sulfate, filtered, and concentrated in vacuo resulted in 1-(3-{2-[4-amino-7-methyl-5-(4-phenoxyphenyl)-7H-pyrrolo[2,3-d]pyrimidin-6-yl]ethynyl}cyclobutyl)piperidin-4-amine (550 mg, 96.3%) as a yellow amorphous solid.

### N-(1-(3-((4-amino-7-methyl-5-(4-phenoxyphenyl)-7H-pyrrolo[2,3-d]pyrimidin-6-yl)ethynyl)cyclobutyl)piperidin-4-yl)acrylamide

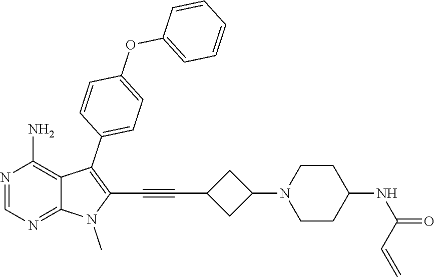 [SMILES: C=CC(=O)NC1CCN(C2CC(C#Cc3c(-c4ccc(Oc5ccccc5)cc4)c4c(N)ncnc4n3C)C2)CC1]

### Step 6

A round bottomed flask was charged with 1-(3-{2-[4-amino-7-methyl-5-(4-phenoxyphenyl)-7H-pyrrolo[2,3-d]pyrimidin-6-yl]ethynyl}cyclobutyl)piperidin-4-amine (300 mg, 608 μmol), DCM (100 mL), TEA (183 mg, 1.82 mmol) and a stirbar. The mixture was stirred and cooled to 0° C., then prop-2-enoyl chloride (49.5 mg, 547 μmol, dissolved in DCM (10 mL)) was added, and the solution was stirred for 1 h at room temperature. The reaction mixture was quenched with MeOH (5 mL), and the solution was concentrated in vacuo. The resulting crude material was purified by HPLC Column: Xselect CSH OBD Column 30*150 mm 5 um n; Mobile Phase A:Water (0.1% FA), Mobile Phase B: ACN; Flow rate: 60 mL/min; Gradient: 5% B to 35% B in 7 min; 220 nm; Rt: 6.17 min. Lyophilization yielded N-[1-(3-{2-[4-amino-7-methyl-5-(4-phenoxyphenyl)-7H-pyrrolo[2,3-d]pyrimidin-6-yl]ethynyl}cyclobutyl)piperidin-4-yl]prop-2-enamide (130 mg, 39.1%) as a white amorphous solid.

Characterization data for the product are listed in Table 14, below.

TABLE 14
Characterization Data

| | | Proton NMR & |
|---|---|---|
| Compound | Structure | Mass Spec. (obs.) |
| N-(1-(3-((4-amino-7-methyl- 5-(4-phenoxyphenyl)-7H- pyrrolo[2,3-d]pyrimidin-6- yl)ethynyl)cyclobutyl) piperidin-4-yl)acrylamide | 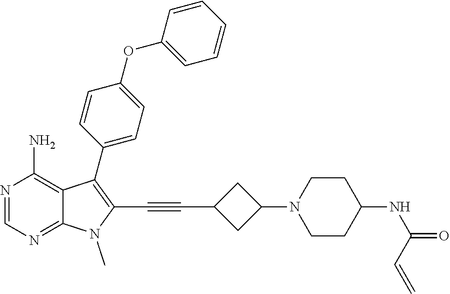 [SMILES: C=CC(=O)NC1CCN(C2CC(C#Cc3c(-c4ccc(Oc5ccccc5)cc4)c4c(N)ncnc4n3C)C2)CC1] | ¹H NMR (400 MHz, DMSO-d₆) δ 8.19 (d, J = 18.1 Hz, 1H), 8.14 (s, 1H), 7.51 (d, J = 8.2 Hz, 2H), 7.42 (t, J = 7.8 Hz, 2H), 7.18 (t, J = 7.3 Hz, 1H), 7.13 (d, J = 8.0 Hz, 4H), 6.50- 6.25 (br, 1H), 6.22 (dd, J = 17.0, 10.0 Hz, 1H), 6.08 (d, J = 16.5 Hz, 1H), 5.58 (d, J = 10.3 Hz, 1H), 3.72 (s, 3H), 3.64 (s, 1H), 3.00 (s, 1H), 2.68 (s, 3H), 2.47 (s, 2H), 1.85 (s, 3H) 1.79 (s, 3H), 1.42 (s, 2H). MS: 547.30 |

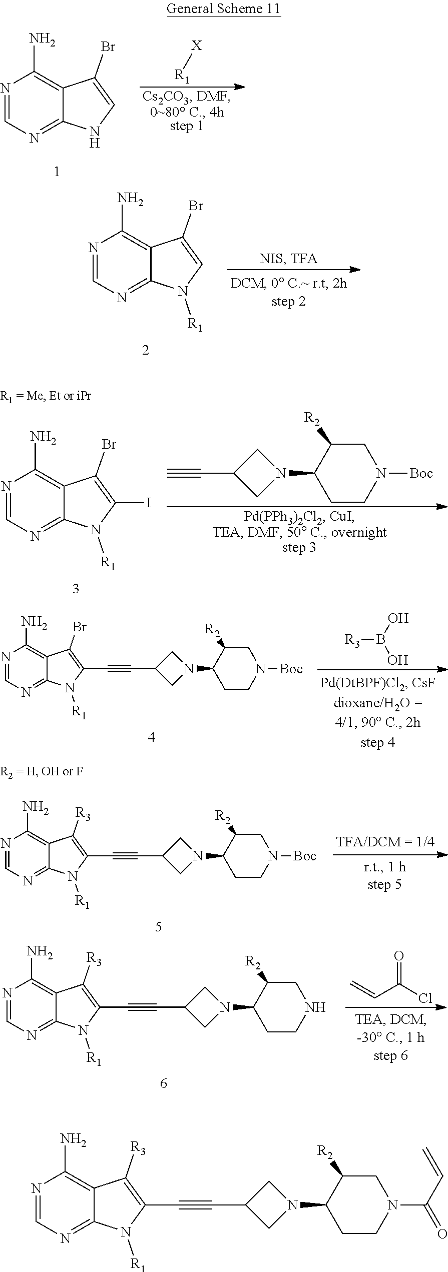

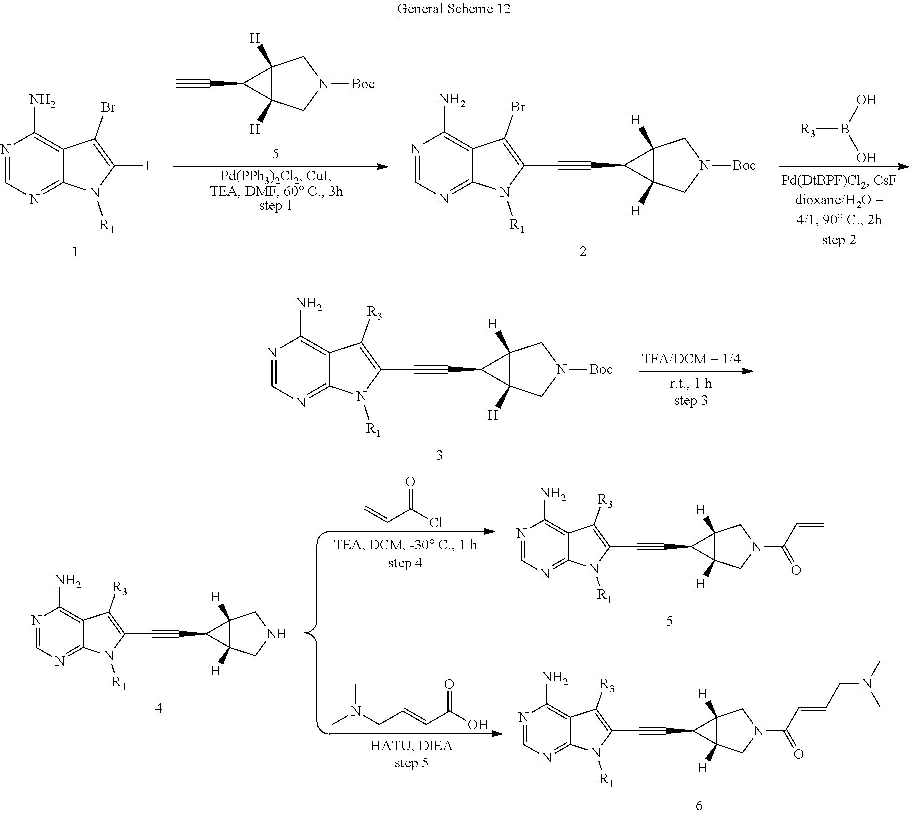

### Example 20

### 1-((3S,4R)-4-(3-((4-amino-7-methyl-5-(1-methyl-1H-indazol-4-yl)-7H-pyrrolo[2,3-d]pyrimidin-6-yl)ethynyl)azetidin-1-yl)-3-hydroxypiperidin-1-yl)prop-2-en-1-one (Compound II-63)

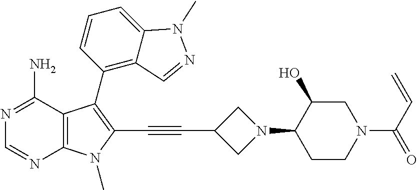 [SMILES: C=CC(=O)N1CC[C@@H](N2CC(C#Cc3c(-c4cccc5c4cnn5C)c4c(N)ncnc4n3C)C2)[C@@H](O)C1]

### 5-bromo-7-methyl-7H-pyrrolo[2,3-d]pyrimidin-4-amine

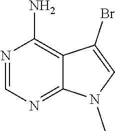

### Step 1

A round bottomed flask was charged with 5-bromo-7H-pyrrolo[2,3-d]pyrimidin-4-amine (3 g, 14.0 mmol), Cs₂CO₃ (9.10 g, 28.0 mmol), DMF (50 mL) and a stir bar. The mixture was cooled to 0° C. and iodomethane (1.98 g, 14.0 mmol) was added, and the solution was stirred for 3 h at 0° C. The reaction mixture was diluted with H₂O (300 mL), and the aqueous phase was extracted with EA (300 mL) three times. The combined organic layers were washed with brines, dried over sodium sulfate, filtered, and concentrated in vacuo. The resulting crude material was purified by silica gel chromatography (eluting with MeOH/DCM=1/80). Concentration in vacuo resulted in 5-bromo-7-methyl-7H-pyrrolo[2,3-d]pyrimidin-4-amine (2.20 g, 70%).

### 5-bromo-6-iodo-7-methyl-7H-pyrrolo[2,3-d]pyrimidin-4-amine

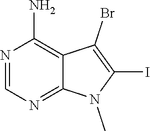

### Step 2

A round bottomed flask was charged with 5-bromo-7-methyl-7H-pyrrolo[2,3-d]pyrimidin-4-amine (3 g, 13.2 mmol), TFA (7.52 g, 66.0 mmol), DCM (50 mL) and a stir bar. The mixture was cooled to 0° C. and 1-iodopyrrolidine-2,5-dione (2.96 g, 13.2 mmol) was added, and the solution was stirred for 2 h at room temperature. The reaction mixture was diluted with saturated Na₂SO₃ solution (100 mL), The pH of the solution was adjusted to 7-8 with saturated Na₂SO₃ solution. The solid was filtered and washed with H₂O, then washed with a small amount of DCM and resulted in 5-bromo-6-iodo-7-methyl-7H-pyrrolo[2,3-d]pyrimidin-4-amine (3.70 g, 80%).

### (3S,4R)-tert-butyl 4-(3-((4-amino-5-bromo-7-methyl-7H-pyrrolo[2,3-d]pyrimidin-6-yl)ethynyl)azetidin-1-yl)-3-hydroxypiperidine-1-carboxylate

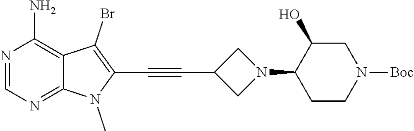 [SMILES: Cn1c(C#CC2CN([C@@H]3CCN(C(=O)OC(C)(C)C)C[C@@H]3O)C2)c(Br)c2c(N)ncnc21]

### Step 3

A resealable reaction vial was charged with 5-bromo-6-iodo-7-methyl-7H-pyrrolo[2,3-d]pyrimidin-4-amine (2 g, 5.66 mmol), tert-butyl (3S,4R)-4-(3-ethynylazetidin-1-yl)-3-hydroxypiperidine-1-carboxylate (1.90 g, 6.79 mmol), Pd(PPh₃)₂Cl₂ (790 mg, 1.13 mmol), CuI (429 mg, 2.26 mmol), DMF (20 mL), TEA (2.84 g, 28.2 mmol) and a stir bar before being evacuated and purged with nitrogen three times. The mixture was stirred for 2 h at 50° C. The reaction mixture was diluted with H₂O (150 mL), and the aqueous phase was extracted with EA (300 mL) three times. The combined organic layers were washed with brines, dried over sodium sulfate, filtered, and concentrated in vacuo. The resulting crude material was purified by silica gel chromatography (eluting with MeOH/DCM=1/30). Concentration in vacuo resulted in tert-butyl (3S,4R)-tert-butyl 4-(3-((4-amino-5-bromo-7-methyl-7H-pyrrolo[2,3-d]pyrimidin-6-yl)ethynyl)azetidin-1-yl)-3-hydroxypiperidine-1-carboxylate (2.0 g, 70%).

### (3S,4R)-tert-butyl 4-(3-((4-amino-7-methyl-5-(1-methyl-1H-indazol-4-yl)-7H-pyrrolo[2,3-d]pyrimidin-6-yl)ethynyl)azetidin-1-yl)-3-hydroxypiperidine-1-carboxylate

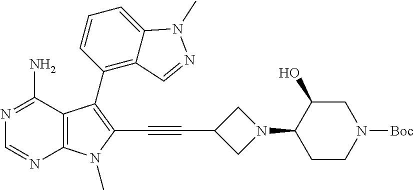 [SMILES: Cn1ncc2c(-c3c(C#CC4CN([C@@H]5CCN(C(=O)OC(C)(C)C)C[C@@H]5O)C4)n(C)c4ncnc(N)c34)cccc21]

### Step 4

A resealable reaction vial was charged with tert-butyl (3S,4R)-4-[3-(2-{4-amino-5-bromo-7-methyl-7H-pyrrolo[2,3-d]pyrimidin-6-yl}ethynyl)azetidin-1-yl]-3-hydroxypiperidine-1-carboxylate (200 mg, 395 μmol), (1-methyl-1H-indazol-4-yl)boronic acid (83.4 mg, 474 μmol), Pd(DtBPF)Cl₂ (25.7 mg, 39.5 μmol), CsF (240 mg, 1.58 mmol), dioxane (6 mL), H2O (1.5 mL) and a stir bar before being evacuated and purged with nitrogen three times. The mixture was stirred for 2 h at 90° C. The reaction mixture was concentrated in vacuo. The resulting crude material was purified by TLC (eluting with MeOH/DCM=1/15). Concentration in vacuo resulted in tert-butyl (3S,4R)-4-(3-{2-[4-amino-7-methyl-5-(1-methyl-1H-indazol-4-yl)-7H-pyrrolo[2,3-d]pyrimidin-6-yl]ethynyl}azetidin-1-yl)-3-hydroxypiperidine-1-carboxylate (160 mg, 72.6%).

### (3S,4R)-4-(3-((4-amino-7-methyl-5-(1-methyl-1H-indazol-4-yl)-7H-pyrrolo[2,3-d]pyrimidin-6-yl)ethynyl)azetidin-1-yl)piperidin-3-ol

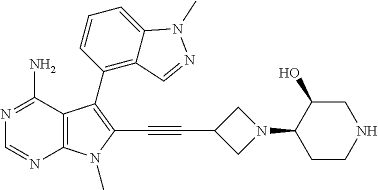 [SMILES: Cn1ncc2c(-c3c(C#CC4CN([C@@H]5CCNC[C@@H]5O)C4)n(C)c4ncnc(N)c34)cccc21]

### Step 5

A round bottomed flask was charged with tert-butyl (3S,4R)-4-(3-{2-[4-amino-7-methyl-5-(1-methyl-1H-indazol-4-yl)-7H-pyrrolo[2,3-d]pyrimidin-6-yl]ethynyl}azetidin-1-yl)-3-hydroxypiperidine-1-carboxylate (150 mg, 269 μmol), DCM (2 mL), TFA (0.5 mL) and a stirbar. The solution was stirred for 1 h at room temperature. The solution was concentration in vacuo resulted in crude (3S,4R)-4-(3-{2-[4-amino-7-methyl-5-(1-methyl-1H-indazol-4-yl)-7H-pyrrolo[2,3-d]pyrimidin-6-yl]ethynyl}azetidin-1-yl)piperidin-3-ol, which was used in the next step directly without further purification.

### 1-((3S,4R)-4-(3-((4-amino-7-methyl-5-(1-methyl-1H-indazol-4-yl)-7H-pyrrolo[2,3-d]pyrimidin-6-yl)ethynyl)azetidin-1-yl)-3-hydroxypiperidin-1-yl)prop-2-en-1-one

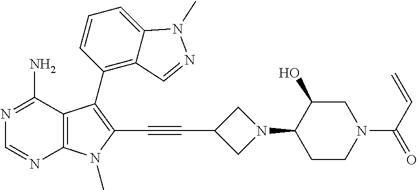 [SMILES: C=CC(=O)N1CC[C@@H](N2CC(C#Cc3c(-c4cccc5c4cnn5C)c4c(N)ncnc4n3C)C2)[C@@H](O)C1]

### Step 6

A round bottomed flask was charged with (3S,4R)-4-(3-{2-[4-amino-7-methyl-5-(1-methyl-1H-indazol-4-yl)-7H-pyrrolo[2,3-d]pyrimidin-6-yl]ethynyl}azetidin-1-yl)piperidin-3-ol (280 mg, 613 μmol), DCM (15 mL) and a stir bar. The pH of the mixture was adjusted to 9-10 with TEA and cooled to −30° C. prop-2-enoyl chloride (22.1 mg, 245 μmol) was added and the solution was stirred for 0.5 h at −30° C. The reaction mixture was quenched with MeOH, and concentrated in vacuo. The resulting crude material was purified by HPLC Column: XBridge Shield RP18 OBD Column 30*150 mm, 5 um; Mobile Phase A:Water (10 MMOL/L NH4HCO3), Mobile Phase B: ACN; Flow rate: 60 mL/min; Gradient: 15% B to 42% B in 7 min; 254 nm; Rt: 5.8 m Lyophilization yielded 1-[(3S,4R)-4-(3-{2-[4-amino-7-methyl-5-(1-methyl-5H-indazol-4-yl)-7H-pyrrolo[2,3-d]pyrimidin-6-yl]ethynyl}azetidin-1-yl)-3-hydroxypiperidin-1-yl]prop-2-en-1-one (77.0 mg, 50.2%).

Compounds prepared by substantially the same methods, and their characterization data, are listed in Table 15, below.

TABLE 15
Selected Compounds

| | | | MS |
|---|---|---|---|
| | | | [M + |
| Compound | Structure | Proton NMR | 1] |
| 1-[4-(3-[2-[4- amino-5-(3- methoxy-4- phenoxyphenyl)- 7-methyl-7H- pyrrolo[2,3- d]pyrimidin-6- yl]ethynyl] azetidin-1- yl)piperidin-1- yl]prop-2-en-1- one | 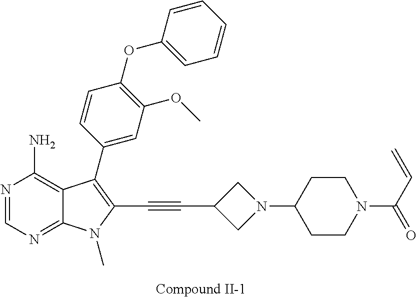 [SMILES: C=CC(=O)N1CCC(N2CC(C#Cc3c(-c4ccc(Oc5ccccc5)c(OC)c4)c4c(N)ncnc4n3C)C2)CC1] | ¹H NMR (400 MHz, DMSO-d₆) δ 8.20 (s, 1H), 7.35 (t, J = 7.9 Hz, 2H), 7.28 (s, 1H), 7.10 (dt, J = 22.1, 7.7 Hz, 3H), 6.96 (d, J = 8.0 Hz, 2H), 6.77 (dd, J = 16.7, 10.5 Hz, 1H), 6.34 (br, 1H), 6.06 (d, J = 16.6 Hz, 1H), 5.64 (d, J = 10.5 Hz, 1H), 3.97 (m, 1H), 3.80 (s, 4H), 3.73 (s, 3H), 3.56 (t, J = 6.7 Hz, 2H), 3.47 (q, J = 7.4 Hz, 1H), 3.14 (t, 1H), 3.06 − 2.89 (m, 3H), 2.24 (m, 1H), 1.59 (m, 2H), 1.05 (m, 2H). | 563.25 |
| 1-(4-(3-((4- amino-7- methyl-5-(2- methyl-4- phenoxyphenyl)- 7H- pyrrolo[2,3- d]pyrimidin-6- yl)ethynyl) azetidin-1- yl)piperidin-1- yl)prop-2-en-1- one | 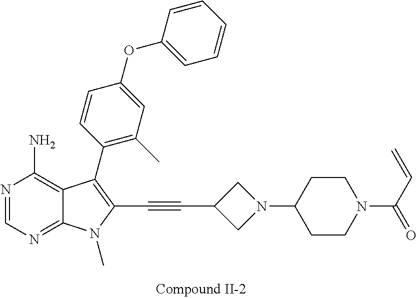 [SMILES: C=CC(=O)N1CCC(N2CC(C#Cc3c(-c4ccc(Oc5ccccc5)cc4C)c4c(N)ncnc4n3C)C2)CC1] | ¹H NMR (400 MHz, Methanol-d₄) δ 8.18 (s, 1H), 7.46 − 7.37 (m, 2H), 7.30 (d, J = 8.3 Hz, 1H), 7.18 (t, J = 7.4 Hz, 1H), 7.13 − 7.07 (m, 2H), 7.03 (d, J = 2.5 Hz, 1H), 6.94 (dd, J = 8.4, 2.6 Hz, 1H), 6.76 (dd, J = 16.8, 10.7 Hz, 1H), 6.18 (dd, J = 16.8, 1.9 Hz, 1H), 5.73 (dd, J = 10.6, 2.0 Hz, 1H), 4.36 (d, J = 12.2 Hz, 1H), 4.03 (d, J = 14.0 Hz, 1H), 3.83 (s, 3H), 3.67 (t, J = 7.3 Hz, 2H), 3.46 (q, J = 7.8 Hz, 1H), 3.25 − 3.10 (m, 3H), 2.92 (t, J = 12.1 Hz, 1H), 2.44 − 2.34 (m, 1H), 2.22 (s, 3H), 1.79 (d, J = 12.9 Hz, 2H), 1.22 − 1.12 (m, 2H). | 547.30 |
| 2-[4-amino-7- methyl-6-(2-[1- [1-(prop-2- enoyl)piperidin- 4-yl]azetidin-3- yl]ethynyl)-7H- pyrrolo[2,3- d]pyrimidin-5- yl]-5- phenoxybenzonitrile | 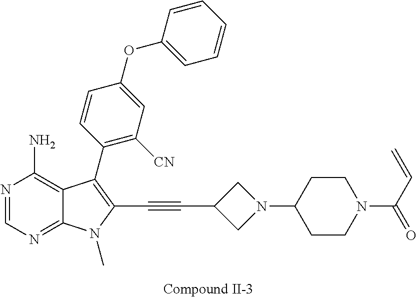 [SMILES: C=CC(=O)N1CCC(N2CC(C#Cc3c(-c4ccc(Oc5ccccc5)cc4C#N)c4c(N)ncnc4n3C)C2)CC1] | ¹H NMR (400 MHz, DMSO-d₆) δ 8.21 (s, 1H), 7.60 (d, J = 2.6 Hz, 1H), 7.49 (dt, 3H), 7.39 (dd, J = 8.6, 2.7 Hz, 1H), 7.24 (t, J = 7.4 Hz, 1H), 7.22 − 7.16 (m, 2H), 6.78 (dd, J = 16.7, 10.5 Hz, 1H), 6.35 (br, 1H), 6.07 (dd, J = 16.7, 2.5 Hz, 1H), 5.64 (dd, J = 10.5, 2.5 Hz, 1H), 3.95 (d, J = 13.2 Hz, 1H), 3.80 (d, J = 15.2 Hz, 1H), 3.75 (s, 3H), 3.54 (q, J = 6.7 Hz, 2H), 3.40 (p, J = 7.5 Hz, 1H), 3.18 (t, J = 11.8 Hz, 1H), 2.97 (dt, J = 22.4, 6.6 Hz, 3H), 2.23 (dt, J = 8.7, 4.6 Hz, 1H), 1.58 (m, 2H), 1.06 (m, 2H). | 558.25 |
| 1-(4-(3-((4- amino-5-(4-((6- methylpyridin- 2- yl)oxy)phenyl)- 7- (tetrahydrofuran- 3-yl)-7H- pyrrolo[2,3- d]pyrimidin-6- yl)ethynyl) azetidin-1- yl)piperidin-1- yl)prop-2-en-1- one | 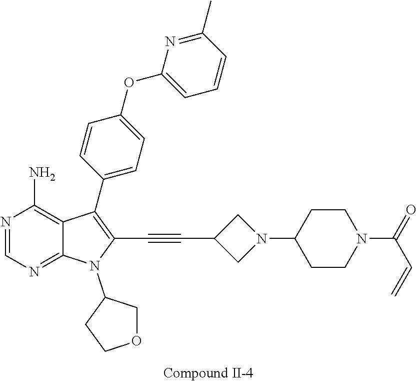 [SMILES: C=CC(=O)N1CCC(N2CC(C#Cc3c(-c4ccc(Oc5cccc(C)n5)cc4)c4c(N)ncnc4n3C3CCOC3)C2)CC1] | ¹H NMR (400 MHz, DMSO-d₆) δ 8.20 (s, 1H), 7.76 (t, J = 7.7 Hz, 1H), 7.54 (d, J = 8.1 Hz, 2H), 7.24 (d, J = 8.1 Hz, 2H), 7.04 (d, J = 7.4 Hz, 1H), 6.84 (d, J = 8.2 Hz, 1H), 6.77 (dd, J = 16.7, 10.5 Hz, 1H), 6.07 (dd, J = 17.0, 2.1 Hz, 1H), 5.69 − 5.60 (m, 1H), 5.53 (p, J = 7.9 Hz, 1H), 4.23 (q, J = 7.7 Hz, 1H), 4.15 − 4.04 (m, 2H), 3.94 (q, J = 7.6 Hz, 2H), 3.81 (s, 1H), 3.59(S, 2H), 3.46 (d, J = 10.0 Hz, 1H), 3.20 − 3.00 (m, 3H), 2.94 (s, 1H), 2.63 (dq, J = 14.0, 7.2 Hz, 1H), 2.37 (s, 5H), 1.61 (s, 2H), 1.07 (s, 2H) | 603.70 |
| 1-(4-(3-((4- amino-5-(4- phenoxyphenyl)- 7H- pyrrolo[2,3- d]pyrimidin-6- yl)ethynyl) azetidin-1- yl)piperidin-1- yl)prop-2-en-1- one | 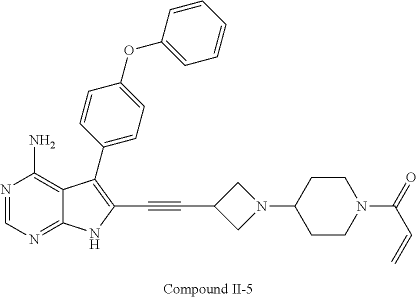 [SMILES: C=CC(=O)N1CCC(N2CC(C#Cc3[nH]c4ncnc(N)c4c3-c3ccc(Oc4ccccc4)cc3)C2)CC1] | ¹H NMR (400 MHz, DMSO-d₆) δ 12.15 (s, 1H), 8.14 (d, J = 5.5 Hz, 2H), 7.52 (d, J = 8.2 Hz, 2H), 7.43 (t, J = 7.7 Hz, 2H), 7.18 (t, J = 7.4 Hz, 1H), 7.12 (dd, J = 8.5, 3.7 Hz, 4H), 6.78 (dd, J = 16.6, 10.4 Hz, 1H), 6.07 (dd, J = 16.8, 2.3 Hz, 1H), 5.65 (dd, J = 10.9, 2.4 Hz, 1H), 4.00 (d, J = 13.0 Hz, 1H), 3.83 (d, J = 13.7 Hz, 1H), 3.58 (m, 2H), 3.40 (t, J = 7.5 Hz, 1H), 3.14 (t, J = 11.5 Hz, 1H), 3.05 (s, 2H), 2.96 (d, J = 12.0 Hz, 1H), 2.31 (s, 1H), 1.61 (s, 2H), 1.06 (s, 2H). | 519.30 |
| 1-(4-(3-((4- amino-5-(3- chloro-4-(6- methylpyridin-2- yloxy)phenyl)- 7-methyl-7H- pyrrolo[2,3- d]pyrimidin-6- yl)ethynyl) azetidin-1- yl)piperidin-1- yl)prop-2-en-1- one | 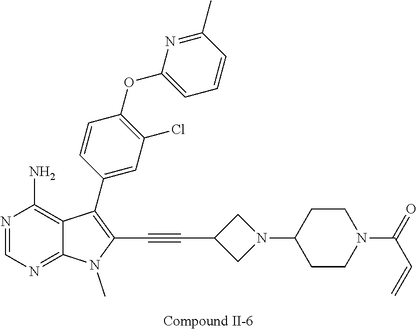 [SMILES: C=CC(=O)N1CCC(N2CC(C#Cc3c(-c4ccc(Oc5cccc(C)n5)c(Cl)c4)c4c(N)ncnc4n3C)C2)CC1] | ¹H NMR (400 MHz, DMSO-d₆) δ 8.20 (s, 1H), 7.81 − 7.73 (m, 1H), 7.73 (d, J = 2.1 Hz, 1H), 7.49 (dd, J = 8.4, 2.1 Hz, 1H), 7.40 (d, J = 8.3 Hz, 1H), 7.04 (d, J = 7.4 Hz, 1H), 6.88 (d, J = 8.1 Hz, 1H), 6.77 (dd, J = 16.7, 10.5 Hz, 1H), 6.35 (s, 1H), 6.06 (dd, J = 16.7, 2.5 Hz, 1H), 5.64 (dd, J = 10.5, 2.5 Hz, 1H), 3.95 (s, 1H), 3.79 (s, 1H), 3.74 (s, 3H), 3.59 (t, J = 6.9 Hz, 2H), 3.48 (q, J = 7.4 Hz, 1H), 3.12 (s, 1H), 3.04 (t, J = 6.9 Hz, 2H), 2.93 (t, J = 11.0 Hz, 1H), 2.34 (s, 3H), 2.26 (s, 1H), 1.57 (s, 2H), 1.06 (s, 2H). | 582.3 |
| 1-(4-(3-((4- amino-5-(4-((6- chloropyridin-2- yl)oxy)phenyl)- 7-methyl-7H- pyrrolo[2,3- d]pyrimidin-6- yl)ethynyl) azetidin-1- yl)piperidin-1- yl)prop-2-en-1- one | 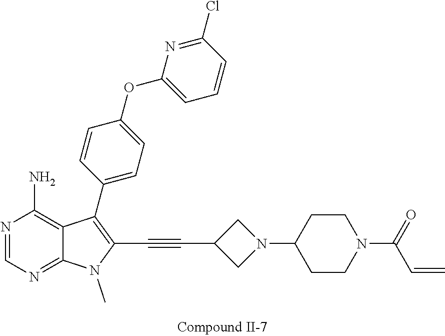 [SMILES: C=CC(=O)N1CCC(N2CC(C#Cc3c(-c4ccc(Oc5cccc(Cl)n5)cc4)c4c(N)ncnc4n3C)C2)CC1] | ¹H NMR (400 MHz, DMSO-d₆) δ 8.21 (s, 1H), 7.94 (t, J = 7.9 Hz, 1H), 7.63 − 7.56 (m, 2H), 7.36 − 7.27 (m, 3H), 7.10 (d, J = 8.1 Hz, 1H), 6.77 (dd, J = 16.7, 10.5 Hz, 1H), 6.06 (dd, J = 16.7, 2.5 Hz, 1H), 5.64 (dd, J = 10.5, 2.5 Hz, 1H), 3.96 (d, J = 13.2 Hz, 1H), 3.80 (d, J = 13.5 Hz, 1H), 3.74 (s, 3H), 3.57 (t, J = 6.9 Hz, 2H), 3.44 (p, J = 7.6 Hz, 1H), 3.21 − 3.10 (m, 1H), 3.03 (t, J = 6.8 Hz, 2H), 2.95 (d, J = 11.7 Hz, 1H), 2.26 (s, 1H), 1.60 (s, 2H), 1.07 (s, 2H). | 568.25 |
| 1-(4-(3-((4- amino-5-(4-((6- fluoropyridin-2- yl)oxy)phenyl)- 7-methyl-7H- pyrrolo[2,3- d]pyrimidin-6- yl)ethynyl) azetidin-1- yl)piperidin-1- yl)prop-2-en-1- one | 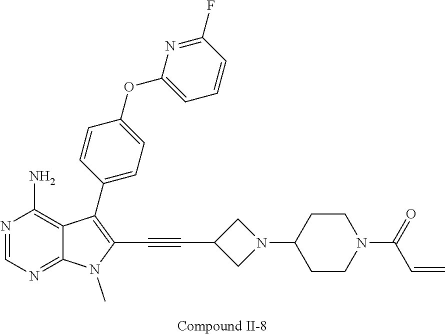 [SMILES: C=CC(=O)N1CCC(N2CC(C#Cc3c(-c4ccc(Oc5cccc(F)n5)cc4)c4c(N)ncnc4n3C)C2)CC1] | ¹H NMR (400 MHz, DMSO-d₆) δ 8.21 (s, 1H), 8.06 (q, J = 8.2 Hz, 1H), 7.62 − 7.56 (m, 2H), 7.36 − 7.30 (m, 2H), 7.02 (dd, J = 8.0, 1.7 Hz, 1H), 6.93 (dd, J = 7.9, 2.4 Hz, 1H), 6.78 (dd, J = 16.7, 10.5 Hz, 1H), 6.07 (dd, J = 16.6, 2.5 Hz, 1H), 5.64 (dd, J = 10.4, 2.4 Hz, 1H), 3.95 (s, 1H), 3.78 (m, 1H), 3.74 (s, 3H), 3.57 (t, J = 7.0 Hz, 2H), 3.45 (q, J = 7.4 Hz, 1H), 3.17 (t, 1H), 3.03 (t, J = 6.9 Hz, 2H), 2.95 (d, J = 11.1 Hz, 1H), 2.27 (t, 1H), 1.59 (s, 2H), 1.06 (s, 2H). | 552.35 |
| 1-(4-(3-((4- amino-7- methyl-5-(4-((2- methylpyridin- 3- yl)oxy)phenyl)- 7H-pyrrolo[2,3- d]pyrimidin-6- yl)ethynyl) azetidin-1- yl)piperidin-1- yl)prop-2-en-1- one | 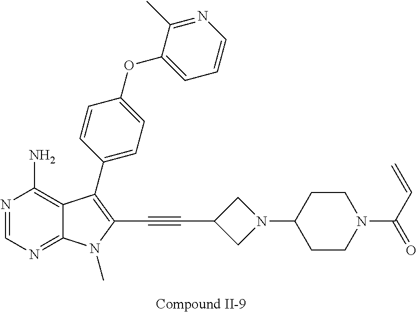 [SMILES: C=CC(=O)N1CCC(N2CC(C#Cc3c(-c4ccc(Oc5cccnc5C)cc4)c4c(N)ncnc4n3C)C2)CC1] | ¹H NMR (400 MHz, Methanol-d₄) δ 8.28 (d, J = 4.7 Hz, 1H), 8.20 (s, 1H), 7.60 (d, J = 8.3 Hz, 2H), 7.48 (d, J = 8.3 Hz, 1H), 7.37 − 7.30 (m, 1H), 7.14 (d, J = 8.3 Hz, 2H), 6.77 (dd, J = 16.8, 10.8 Hz, 1H), 6.18 (d, J = 17.7 Hz, 1H), 5.74 (d, J = 11.1 Hz, 1H), 4.35 (s, 1H), 4.04 (d, J = 13.7 Hz, 1H), 3.83 (s, 3H), 3.69 (t, J = 7.2 Hz, 2H), 3.52 (d, J = 7.6 Hz, 1H), 3.22 (t, J = 7.1 Hz, 3H), 2.93 (s, 1H), 2.55 (s, 3H), 2.41 (s, 1H), 1.80 (d, J = 13.1 Hz, 2H), 1.19 (s, 2H). | 548.3 |
| 1-(4-(3-((4- amino-7- methyl-5-(4-(1- methylcyclopropoxy) phenyl)- 7H-pyrrolo[2,3- d]pyrimidin-6- yl)ethynyl) azetidin-1- yl)piperidin-1- yl)prop-2-en-1- one | 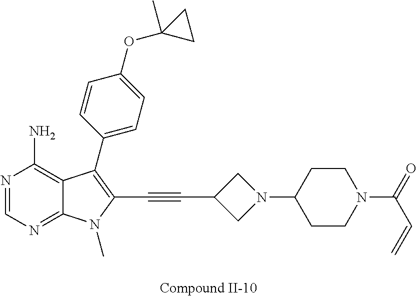 [SMILES: C=CC(=O)N1CCC(N2CC(C#Cc3c(-c4ccc(OC5(C)CC5)cc4)c4c(N)ncnc4n3C)C2)CC1] | ¹H NMR (400 MHz, Chloroform-d) δ 8.33 (s, 1H), 7.49 − 7.48 (m, 2H), 7.14 − 7.13 (m, 2H), 6.56 (dd, J = 16.8, 10.6 Hz, 1H), 6.27 (dd, J = 16.9, 2.0 Hz, 1H), 5.68(dd, J = 10.6, 1.9 Hz, 1H), 5.26 (s, 2H), 4.25 (d, J = 13.2 Hz, 1H), 3.85 (s, 4H), 3.69 (t, J = 7.1 Hz, 2H), 3.47 (q, J = 7.8 Hz, 1H), 3.23 − 3.18 (m, 1H), 3.09 (t, J = 7.1 Hz, 3H), 2.33 (dt, J = 8.9, 5.0 Hz, 1H), 1.71 (d, J = 13.0 Hz, 2H), 1.63 (s, 3H), 1.28 (dtd, J = 13.3, 9.3, 4.0 Hz, 2H), 1.08 − 1.05 (m, 2H), 0.80 − 0.77 (m, 2H). | 511.3 |
| 1-(4-(3-((4- amino-5-(5- chloro-6- phenoxypyridin- 3-yl)-7-methyl- 7H-pyrrolo[2,3- d]pyrimidin-6- yl)ethynyl) azetidin-1- yl)piperidin-1- yl)prop-2-en-1- one | 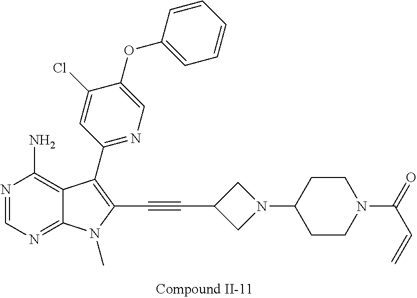 [SMILES: C=CC(=O)N1CCC(N2CC(C#Cc3c(-c4cc(Cl)c(Oc5ccccc5)cn4)c4c(N)ncnc4n3C)C2)CC1] | ¹H NMR (400 MHz, DMSO-d₆) δ 8.20 (s, 1H), 8.17 − 8.07 (m, 2H), 7.46 (t, J = 7.8 Hz, 2H), 7.30 − 7.17 (m, 3H), 6.78 (dd, J = 16.7, 10.5 Hz, 1H), 6.51 (s, 2H), 6.07 (dd, J = 16.7, 2.5 Hz, 1H), 5.65 (dd, J = 10.5, 2.5 Hz, 1H), 3.95 (s, 1H), 3.80 (s, 1H), 3.72 (s, 3H), 3.57 (t, J = 6.9 Hz, 2H), 3.45 (t, J = 7.5 Hz, 1H), 3.18 (s, 1H), 3.02 (t, J = 6.7 Hz, 3H), 2.27 (s, 1H), 1.59 (s, 2H), 1.09 (s, 2H). | 568.20 |
| 1-((3S,4R)-4-(3- ((4-amino-5-(4- (isothiazol-3- yloxy)phenyl)- 7-methyl-7H- pyrrolo[2,3- d]pyrimidin-6- yl)ethynyl)azetidin- 1-yl)-3- hydroxypiperidin- 1-yl)prop-2- en-1-one | 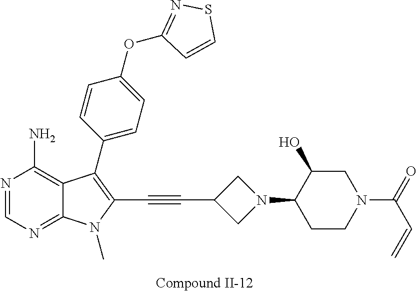 [SMILES: C=CC(=O)N1CC[C@@H](N2CC(C#Cc3c(-c4ccc(Oc5ccsn5)cc4)c4c(N)ncnc4n3C)C2)[C@@H](O)C1] | ¹H NMR (400 MHz, DMSO-d₆) δ 9.06 (d, J = 4.7 Hz, 1H), 8.20 (s, 1H), 7.60 − 7.52 (m, 2H), 7.38 (d, J = 8.3 Hz, 2H), 7.04 (d, J = 4.7 Hz, 1H), 6.74 (td, J = 17.7, 17.3, 10.3 Hz, 1H), 6.21 (s, 2H), 6.09 − 6.00 (m, 1H), 5.62 (d, J = 9.8 Hz, 1H), 4.39 (d, J = 3.2 Hz, 1H), 4.02 (d, J = 12.7 Hz, 1H), 3.98 − 3.52 (m, 7H), 3.45 (p, J = 7.5 Hz, 1H), 3.33 − 3.06 (m, 3H), 2.90 − 2.82 (m, 1H), 2.28 (s, 1H), 1.37 (d, J = 5.2 Hz, 2H). | 556.3 |
| 1-((3S,4R)-4-(3- ((4-amino-7- methyl-5-(4- (pyridin-2- yloxy)phenyl)- 7H-pyrrolo[2,3- d]pyrimidin-6- yl)ethynyl) azetidin-1-yl)-3- hydroxypiperidin- 1-yl)prop-2- en-1-one | 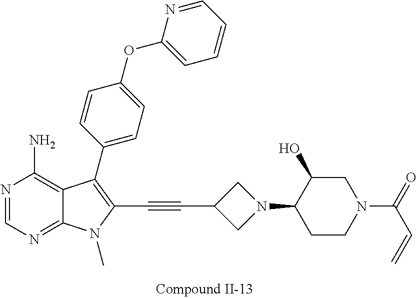 [SMILES: C=CC(=O)N1CC[C@@H](N2CC(C#Cc3c(-c4ccc(Oc5ccccn5)cc4)c4c(N)ncnc4n3C)C2)[C@@H](O)C1] | ¹H NMR (400 MHz, DMSO-d₆) δ 8.20 (s, 2H), 7.89 (s, 1H), 7.55 (s, 2H), 7.27 (s, 2H), 7.14 (d, J = 30.2 Hz, 2H), 6.72 (s, 1H), 6.16 (s, 1H), 6.04 (d, J = 16.7 Hz, 1H), 5.62 (s, 1H), 4.43 (d, J = 47.8 Hz, 1H), 4.00 (s, 1H), 3.80 − 3.59 (m, 4H), 3.58 (s, 2H), 3.45 (s, 1H), 3.10 (s, 3H), 2.85 (s, 1H), 2.33 (d, J = 30.7 Hz, 1H), 1.43 (d, J = 53.6 Hz, 2H). | 550.3 |
| 1-((3S,4R)-4-(3- ((4-amino-7- methyl-5-(4- (pyrazin-2- yloxy)phenyl)- 7H-pyrrolo[2,3- d]pyrimidin-6-yl) ethynyl)azetidin- 1-yl)-3- hydroxypiperidin- 1-yl)prop-2- en-1-one | 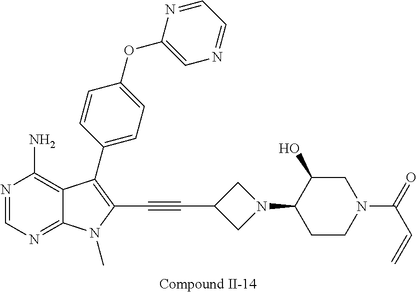 [SMILES: C=CC(=O)N1CC[C@@H](N2CC(C#Cc3c(-c4ccc(Oc5cnccn5)cc4)c4c(N)ncnc4n3C)C2)[C@@H](O)C1] | ¹H NMR (400 MHz, DMSO-d₆) δ 8.61 (s, 1H), 8.43 (s, 1H), 8.28 (s, 1H), 8.21 (d, J = 1.9 Hz, 1H), 7.59 (d, J = 8.1 Hz, 2H), 7.37 (d, J = 8.0 Hz, 2H), 6.72 (dd, J = 17.4, 10.5 Hz, 1H), 6.23 (s, 2H), 6.05 (d, J = 16.7 Hz, 1H), 5.62 (d, J = 10.7 Hz, 1H), 4.51- 4.39 (d, 1H), 4.00 (d, J = 12.6 Hz, 1H), 3.80 − 3.71 (m, 4H), 3.70 − 3.63 (m, 1H), 3.61 − 3.52 (m, 2H), 3.51- 3.39 (d, J = 2.1 Hz, 1H), 3.25 − 2.87 (m, 4H), 2.36 (d, 1H), 1.51- 1.31 (d, 2H). | 551.3 |
| | Compound II-15 | | |
| 1-((3S,4R)-4-(3- ((4-amino-7- methyl-5-(4- (pyridin-3- yloxy)phenyl)- 7H-pyrrolo[2,3- d]pyrimidin-6- yl)ethynyl)azetidin- 1-yl)-3- hydroxypiperidin- 1-yl)prop-2- en-1-one | 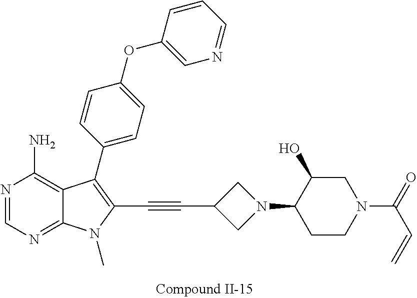 [SMILES: C=CC(=O)N1CC[C@@H](N2CC(C#Cc3c(-c4ccc(Oc5cccnc5)cc4)c4c(N)ncnc4n3C)C2)[C@@H](O)C1] | ¹H NMR (400 MHz, DMSO-d₆) δ 8.48 (s, 1H), 8.40 (s, 1H), 8.20 (s, 1H), 7.56 (m, 3H), 7.48 (m, 1H), 7.21 (m, 2H), 6.75 (m, 1H), 6.25 (s, 2H), 6.06(d, 1H), 5.64(d, 1H), 4.52 − 4.38 (d, 1H), 4.02 (d, 1H), 3.82 (m, 1H), 3.72 (m, 4H), 3.60 (m, 2H), 3.45 (m, 1H), 3.20 (m, 3H), 2.88 (s, 1H), 2.31 (m, 1H), 1.45 (d, 2H). | 550.4 |
| 1-((3S,4R)-4-(3- ((4-amino-7- methyl-5-(4-(1- methyl- cyclopropoxy) phenyl)- 7H-pyrrolo[2,3- d]pyrimidin-6- yl)ethynyl)azeti din-1-yl)-3- hydroxypiperidin- 1-yl)prop-2- en-1-one | 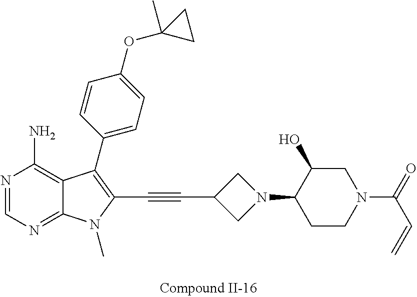 [SMILES: C=CC(=O)N1CC[C@@H](N2CC(C#Cc3c(-c4ccc(OC5(C)CC5)cc4)c4c(N)ncnc4n3C)C2)[C@@H](O)C1] | ¹H NMR (400 MHz, DMSO-d₆) δ 8.18 (s, 1H), 7.47- 7.40 (m, 2H), 7.16 − 7.10 (m, 2H), 6.75 (ddd, J = 21.0, 16.8, 10.4 Hz, 1H), 6.05 (dd, J = 16.7, 2.6 Hz, 2H), 5.67 − 5.58 (m, 1H), 4.58 − 4.35 (m, 1H), 4.02 (d, J = 13.1 Hz, 1H), 3.81-3.50 (m, 7H), 3.44 (q, J = 7.6 Hz, 1H), 3.29-3.13 (m, 1H), 3.15 − 3.03 (m, 2H), 2.93 − 2.85 (m, 1H), 2.41 − 2.29 (m, 1H), 1.55 (s, 3H), 1.38 (s, 2H), 0.94 (q, J = 5.1, 4.6 Hz, 2H), 0.80 (q, J = 5.1 Hz, 2H). | 527.3 |
| 1-((3S,4R)-4-(3- ((4-amino-5-(4- (2-fluorophenoxy) phenyl)-7- methyl-7H- pyrrolo[2,3- d]pyrimidin-6- yl)ethynyl) azetidin-1-yl)-3- hydroxypiperidin- 1-yl)prop-2- en-1-one | 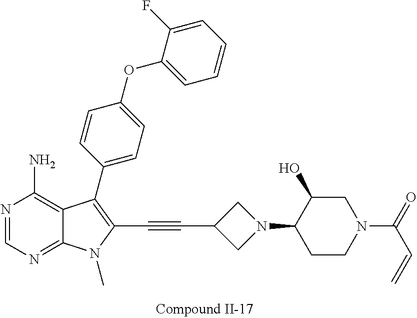 [SMILES: C=CC(=O)N1CC[C@@H](N2CC(C#Cc3c(-c4ccc(Oc5ccccc5F)cc4)c4c(N)ncnc4n3C)C2)[C@@H](O)C1] | ¹H NMR (400 MHz, DMSO-d₆) δ 8.19 (d, J = 1.7 Hz, 1H), 7.55 − 7.48 (m, 2H), 7.43 (t, J = 9.5 Hz, 1H), 7.29 (d, J = 10.7 Hz, 4H), 7.15 − 7.08 (m, 2H), 6.73 (dd, J = 17.7, 10.6 Hz, 1H), 6.19 (s, 2H), 6.05 (d, J = 16.7 Hz, 1H), 5.63 (d, J = 10.6 Hz, 1H), 4.45 (d, J = 49.0 Hz, 1H), 4.02 (s, 1H), 3.72 (d, J = 1.7 Hz, 4H), 3.66 (s, 1H), 3.58 (s, 2H), 3.52 − 3.37 (m, 1H), 3.26 − 3.13 (m, 1H), 3.08 (s, 2H), 2.87 (s, 1H), 2.31 (d, J = 26.7 Hz, 1H), 1.37 (s, 2H), 1.24 (s, 1H). | 567.3 |
| 1-((3S,4R)-4-(3- ((4-amino-5-(4-(3- fluorophenoxy) phenyl)-7- methyl-7H- pyrrolo[2,3- d]pyrimidin-6- yl)ethynyl)azetidin- 1-yl)-3- hydroxypiperidin- 1-yl)prop-2- en-1-one | 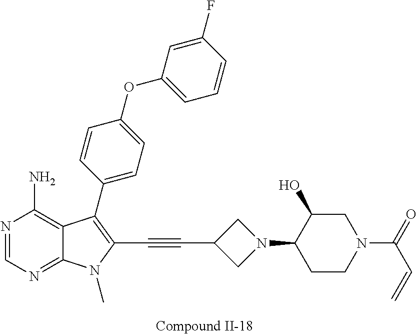 [SMILES: C=CC(=O)N1CC[C@@H](N2CC(C#Cc3c(-c4ccc(Oc5cccc(F)c5)cc4)c4c(N)ncnc4n3C)C2)[C@@H](O)C1] | ¹H NMR (400 MHz, DMSO-d₆) δ 8.19 (d, J = 1.6 Hz, 1H), 7.55 (d, J = 8.1 Hz, 2H), 7.45 (q, J = 7.9 Hz, 1H), 7.20 (d, J = 8.1 Hz, 2H), 7.04 − 6.93 (m, 3H), 6.74 (td, J = 17.5, 10.4 Hz, 1H), 6.26 (s, 1H), 6.05 (d, J = 16.7 Hz, 1H), 5.62 (d, J = 10.4 Hz, 1H), 4.51 − 4.39 (s, 1H), 4.01 (d, J = 12.7 Hz, 1H), 3.83 − 3.60 (m, 5H), 3.55 (s, 2H), 3.44 (p, J = 7.2 Hz, 1H), 3.32 − 3.02 (t, J = 7.8 Hz, 3H), 2.88 (d, J = 13.4 Hz, 1H), 2.35 − 2.28 (s, 1H), 1.50 − 1.37 (s, 2H). | 567.2 |
| 1-((3S,4R)-4-(3- ((4-amino-5-(2- methoxy-4-(6- methylpyridin- 2- yloxy)phenyl)- 7-methyl-7H- pyrrolo[2,3- d]pyrimidin-6- yl)ethynyl)azetidin- 1-yl)-3- hydroxypiperidin- 1-yl)prop-2-en- 1-one | 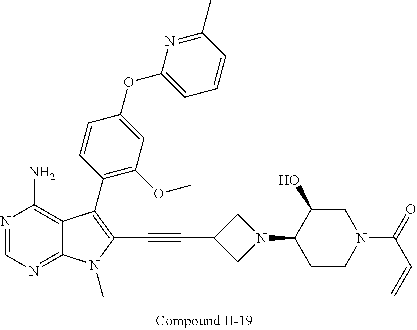 [SMILES: C=CC(=O)N1CC[C@@H](N2CC(C#Cc3c(-c4ccc(Oc5cccc(C)n5)cc4OC)c4c(N)ncnc4n3C)C2)[C@@H](O)C1] | ¹H NMR (400 MHz, DMSO-d₆) δ 8.15 (s, 1H), 7.77 (t, J = 7.7 Hz, 1H), 7.32 (d, J = 8.2 Hz, 1H), 7.05 (d, J = 7.4 Hz, 1H), 6.97 (d, J = 2.3 Hz, 1H), 6.84 (d, J = 8.1 Hz, 1H), 6.82 − 6.64 (m, 2H), 6.04 (dd, J = 16.7, 2.5 Hz, 2H), 5.62 (d, J = 10.2 Hz, 1H), 4.44 (d, J = 47.5 Hz, 1H), 3.99 (s, 1H), 3.80 (s, 1H), 3.73 (d, J = 8.8 Hz, 6H), 3.65 (s, 1H), 3.56 (s, 2H), 3.41 (p, J = 7.8 Hz, 1H), 3.17 (d, J = 13.7 Hz, 1H), 3.03 (d, J = 7.9 Hz, 2H), 2.84 (s, 1H), 2.39 (s, 4H), 2.30 (d, J = 30.5 Hz, 1H), 1.42 (d, J = 56.0 Hz, 2H). | 594.35 |
| 1-((3S,4R)-4-(3- ((4-amino-5-(3- chloro-4-((6- methylpyridin-2- yl)oxy)phenyl)- 7-methyl-7H- pyrrolo[2,3- d]pyrimidin-6- yl)ethynyl) azetidin-1-yl)-3- hydroxypiperidin- 1-yl)prop-2- en-1-one | 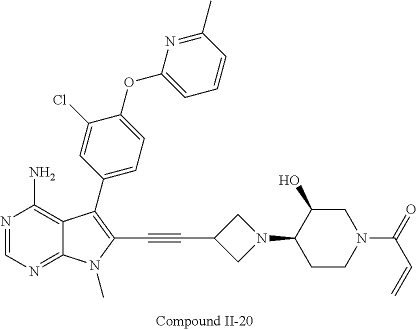 [SMILES: C=CC(=O)N1CC[C@@H](N2CC(C#Cc3c(-c4ccc(Oc5cccc(C)n5)c(Cl)c4)c4c(N)ncnc4n3C)C2)[C@@H](O)C1] | ¹H NMR (400 MHz, DMSO-d₆) δ 8.21 (s, 1H), 7.83 − 7.68 (m, 2H), 7.49 (d, J = 8.5 Hz, 1H), 7.40 (d, J = 8.4 Hz, 1H), 7.04 (d, J = 7.4 Hz, 1H), 6.87 (d, J = 8.2 Hz, 1H), 6.71 (dd, J = 17.2, 10.6 Hz, 1H), 6.35 (s, 2H), 6.04 (d, J = 16.6 Hz, 1H), 5.62 (d, J = 10.0 Hz, 1H), 4.44 (d, J = 45.3 Hz, 1H), 4.01 (d, J = 13.5 Hz, 1H), 3.75 − 3.43 (m, 8H), 3.12 (d, J = 14.9 Hz, 3H), 2.80 (s, 1H), 2.35 (s, 4H), 1.42 (d, J = 58.5 Hz, 2H). | 598.3 |
| 1-((3S,4R)-4-(3- ((4-amino-7- methyl-5-(4- (thiazol-2- yloxy)phenyl)- 7H-pyrrolo[2,3- d]pyrimidin-6- yl)ethynyl)azetidin- 1-yl)-3- hydroxypiperidin- 1-yl)prop-2- en-1-one | 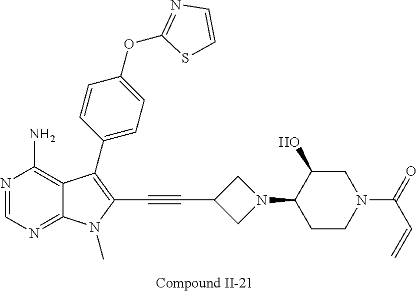 [SMILES: C=CC(=O)N1CC[C@@H](N2CC(C#Cc3c(-c4ccc(Oc5nccs5)cc4)c4c(N)ncnc4n3C)C2)[C@@H](O)C1] | ¹H NMR (400 MHz, DMSO-d₆) δ 8.20 (s, 1H), 7.69 − 7.56 (m, 2H), 7.49 (d, J = 8.3 Hz, 2H), 7.34 (d, J = 3.7 Hz, 1H), 7.29 (d, J = 3.7 Hz, 1H), 6.84 − 6.67 (m, 1H), 6.23 (s, 1H), 6.04 (d, J = 16.8 Hz, 1H), 5.62 (d, J = 10.2 Hz, 1H), 4.45 (d, J = 49.9 Hz, 2H), 4.01 (d, J = 12.8 Hz, 1H), 3.73 (s, 3H), 3.66 (s, 1H), 3.58 (s, 2H), 3.52 − 3.39 (m, 1H), 3.21 − 3.13 (m, 1H), 3.07 (s, 2H), 2.85 (s, 1H), 2.27 (s, 1H), 1.43 (d, J = 54.4 Hz, 2H), 1.24 (s, 1H). | 556.20 |
| 1-((1R,5S,6s)-6- ((4-amino-7- methyl-5-(4- (pyrazin-2- yloxy)phenyl)- 7H-pyrrolo[2,3- d]pyrimidin-6- yl)ethynyl)-3- aza- bicyclo[3.1.0] hexan- 3-yl)prop-2- en-1-one | 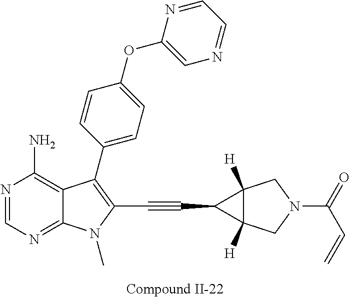 [SMILES: C=CC(=O)N1C[C@@H]2[C@@H](C#Cc3c(-c4ccc(Oc5cnccn5)cc4)c4c(N)ncnc4n3C)[C@@H]2C1] | ¹H NMR (400 MHz, DMSO-d₆) δ 8.62 (d, J = 1.4 Hz, 1H), 8.43 (d, J = 2.7 Hz, 1H), 8.28 (dd, J = 2.7, 1.4 Hz, 1H), 8.19 (s, 1H), 7.62 − 7.49 (m, 2H), 7.37 (d, J = 8.4 Hz, 2H), 6.50 (dd, J = 16.8, 10.4 Hz, 1H), 6.35 (br, 1H), 6.10 (dd, J = 16.8, 2.3 Hz, 1H), 5.65 (dd, J = 10.3, 2.3 Hz, 1H), 3.89 (d, J = 10.7 Hz, 1H), 3.76 (d, J = 12.6 Hz, 1H), 3.72 (s, 3H), 3.64 (dd, J = 10.8, 4.2 Hz, 1H), 3.40 (dd, J = 12.7, 4.4 Hz, 1H), 2.13 (d, J = 3.9 Hz, 1H), 2.10 − 2.02 (m, 1H), 1.49 (d, J = 3.6 Hz, 1H). | 478.15 |
| 1-[(1R,5S,6S)- 6-[2-[4-amino- 7-(oxolan-3-yl)- 5-[4-(pyridin-2- yloxy)phenyl]- 7H-pyrrolo[2,3- d]pyrimidin-6- yl]ethynyl]-3- azabicyclo[3.1.0] hexan-3- yl]prop-2-en-1- one | 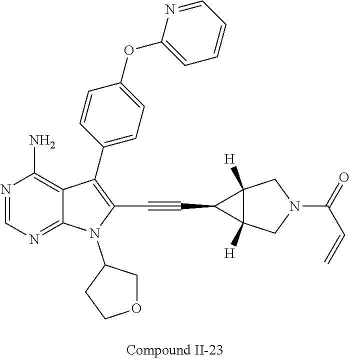 [SMILES: C=CC(=O)N1C[C@@H]2[C@@H](C#Cc3c(-c4ccc(Oc5ccccn5)cc4)c4c(N)ncnc4n3C3CCOC3)[C@@H]2C1] | ¹H NMR (400 MHz, DMSO-d₆) δ 8.23 (s, 1H), 8.22 (d, 1H), 7.88 (t, J = 7.8 Hz, 1H), 7.52 (d, J = 8.1 Hz, 2H), 7.27 (d, J = 8.1 Hz, 2H), 7.18 (dd, J = 7.4 Hz, 1H), 7.12 (d, J = 7.4 Hz, 1H), 6.49 (dd, 1H), 6.10 (d, J = 16.7 Hz, 2H), 5.65 (dd, J = 10.3 Hz, 1H), 5.52 (m, J = 8.1 Hz, 1H), 4.22 (d, J = 7.0 Hz, 1H), 4.07 (d, J = 8.0 Hz, 2H), 3.91 (dd, J = 18.0, 9.3 Hz, 2H), 3.76 (d, J = 12.6 Hz, 1H), 3.63 (dd, J = 7.8 Hz, 1H), 3.39 (d, 1H), 2.58 (dt, J = 14.2, 6.9 Hz, 1H), 2.37 (dt, 1H), 2.09 (d, J = 21.8 Hz, 2H), 1.48 (s, 1H). | 533.20 |
| 1-[(1R,5S,6S)- 6-[2-(4-amino- 5-[4-[(6- methylpyridin- 2- yl)oxy]phenyl]- 7-(oxolan-3-yl)- 7H-pyrrolo[2,3- d]pyrimidin-6- yl)ethynyl]-3- azabicyclo[3.1.0] hexan-3- yl]prop-2-en-1- one | 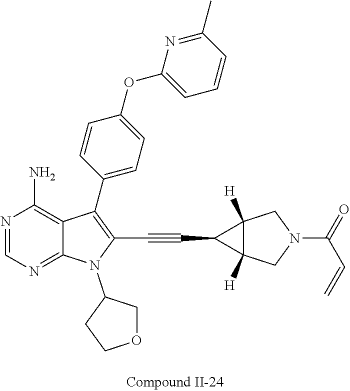 [SMILES: C=CC(=O)N1C[C@@H]2[C@@H](C#Cc3c(-c4ccc(Oc5cccc(C)n5)cc4)c4c(N)ncnc4n3C3CCOC3)[C@@H]2C1] | ¹H NMR (400 MHz, DMSO-d₆) δ 8.19 (s, 1H), 7.77 (t, J = 7.8 Hz, 1H), 7.51 (d, J = 8.1 Hz, 2H), 7.24 (d, J = 8.1 Hz, 2H), 7.05 (d, J = 7.4 Hz, 1H), 6.86 (d, J = 8.2 Hz, 1H), 6.49 (dd, 1H), 6.10 (d, J = 16.7 Hz, 2H), 5.65 (d, J = 10.3 Hz, 1H), 5.51 (p, J = 8.1 Hz, 1H), 4.22 (d, J = 7.0 Hz, 1H), 4.06 (d, J = 8.0 Hz, 2H), 3.91 (dd, J = 18.0, 9.3 Hz, 2H), 3.76 (d, J = 12.6 Hz, 1H), 3.63 (dd, J = 7.8 Hz, 1H), 3.45 − 3.35 (m, 1H), 2.59 (dt, J = 14.2, 6.9 Hz, 1H), 2.37 (s, 4H), 2.08 (d, J = 21.8 Hz, 2H), 1.48 (s, 1H). | 547.20 |
| 1-((3S,4R)-4-(3- ((4-amino-5-(4- methoxyphenyl)- 7-methyl-7H- pyrrolo[2,3- d]pyrimidin-6- yl)ethynyl)azetidin- 1-yl)-3- hydroxypiperidin- 1-yl)prop-2- en-1-one | 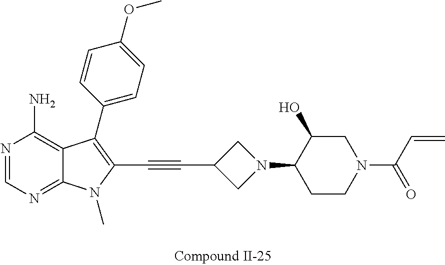 [SMILES: C=CC(=O)N1CC[C@@H](N2CC(C#Cc3c(-c4ccc(OC)cc4)c4c(N)ncnc4n3C)C2)[C@@H](O)C1] | ¹H NMR (400 MHz, DMSO-d₆) δ 8.19 (s, 1H), 7.45 (m, J = 8.1 Hz, 2H), 7.05 (m, J = 1.9 Hz, 2H), 6.75 (m, J = 8.1, 1.9 Hz, 1H), 6.25 (s, 2H), 6.05 (d, 1H), 5.68 (m, 1H), 4.48 (d, J = 3.6 Hz, 1H), 4.02 (m, J = 13.2 Hz, 1H), 3.90 (m, 4H), 3.76 (m, 3H), 3.65 (m, 1H), 3.56 (d, J = 6.5 Hz, 2H), 3.41 (t, J = 6.5 Hz, 1H), 3.18 (m, J = 7.5 Hz,3H), 2.91 (s, 1H), 2.31 (d, , 1H), 1.41 (m, 2H). | 487.3 |
| 1-((3S,4R)-4-(3- ((4-amino-5-(4- chlorophenyl)- 7-methyl-7H- pyrrolo[2,3- d]pyrimidin-6- yl)ethynyl)azetidin- 1-yl)-3- hydroxypiperidin- 1-yl)prop-2- en-1-one | 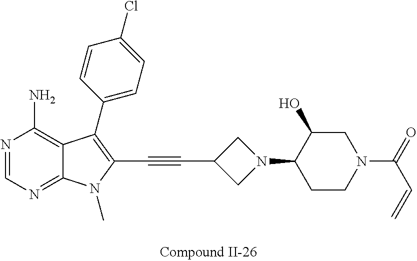 [SMILES: C=CC(=O)N1CC[C@@H](N2CC(C#Cc3c(-c4ccc(Cl)cc4)c4c(N)ncnc4n3C)C2)[C@@H](O)C1] | ¹H NMR (400 MHz, DMSO-d₆) δ 8.20 (s, H), 7.60 − 7.50 (m,4H), 6.84 − 6.67 (m, 1H), 6.21 (s, 2H), 6.05 (d, J = 16.8 Hz, 1H), 5.66 − 5.59 (m, 1H), 4.53- 4.40 (d, J = 3.3 Hz, 1H), 4.02 (d, J = 12.6 Hz, 1H), 3.75 (s, 3H), 3.65 (m, 1H), 3.52 (m, 2H), 3.45 (m, 1H), 3.10 (dt, J = 13.8, 7.0 Hz, 3H), 2.92 (d, J = 7.3 Hz, 1H), 2.30 (s, 1H), 1.40 (d, J = 5.3 Hz, 2H). | 491.3 |
| 1-((1R,5S,6s)-6- ((4-amino-5-(3- methoxy-4- phenoxyphenyl)- 7-methyl-7H- pyrrolo[2,3- d]pyrimidin-6- yl)ethynyl)-3- azabicyclo[3.1.0] hexan-3- yl)prop-2-en-1- one | 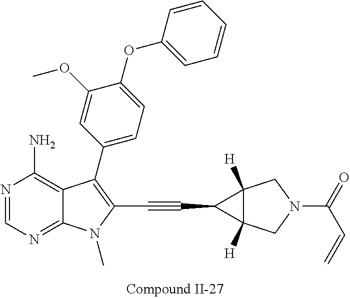 [SMILES: C=CC(=O)N1C[C@@H]2[C@@H](C#Cc3c(-c4ccc(Oc5ccccc5)c(OC)c4)c4c(N)ncnc4n3C)[C@@H]2C1] | ¹H NMR (400 MHz, DMSO-d₆) δ 8.19 (s, 1H), 7.39 − 7.31 (m, 2H), 7.26 (d, J = 1.9 Hz, 1H), 7.13 (d, J = 8.1 Hz, 1H), 7.10 − 7.03 (m, 2H), 7.02 − 6.94 (m, 2H), 6.50 (dd, J = 16.8, 10.3 Hz, 1H), 6.32 (br, 1H), 6.10 (dd, J = 16.8, 2.4 Hz, 1H), 5.66 (dd, J = 10.3, 2.3 Hz, 1H), 3.89 (d, J = 10.8 Hz, 1H), 3.80 (s, 3H), 3.76 (d, J = 12.5 Hz, 1H), 3.71 (s, 3H), 3.64 (dd, J = 10.8, 4.3 Hz, 1H), 3.40 (dd, J = 12.6, 4.3 Hz, 1H), 2.11 (q, J = 3.8 Hz, 1H), 2.04 (dt, J = 7.4, 3.8 Hz, 1H), 1.53 (t, J = 3.5 Hz, 1H). | 506.2 |
| (2E)-1- [(1R,5S,6S)-6- [2-(4-amino-5- [4-[(6- methylpyridin-2- yl)oxy]phenyl]- 7-(oxolan-3-yl)- 7H-pyrrolo[2,3- d]pyrimidin-6- yl)ethynyl]-3- azabicyclo[3.1.0] hexan-3-yl]-4- (dimethylamino) but-2-en-1-one | 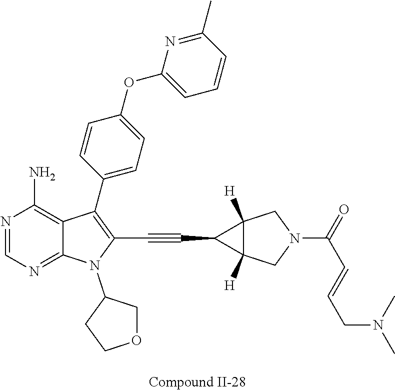 [SMILES: Cc1cccc(Oc2ccc(-c3c(C#C[C@@H]4[C@H]5CN(C(=O)/C=C/CN(C)C)C[C@@H]45)n(C4CCOC4)c4ncnc(N)c34)cc2)n1] | ¹H NMR (400 MHz, DMSO-d₆) δ 8.18 (s, J = 2.1 Hz, 1H), 7.77 (t, J = 7.6 Hz, 1H), 7.51 (d, J = 8.0 Hz, 2H), 7.24 (d, J = 8.1 Hz, 2H), 7.05 (d, J = 7.8 Hz, 1H), 6.86 (d, 1H), 6.57 (dd, J = 14.5, 7.1 Hz, 1H), 6.28 (d, J = 15.2 Hz, 1H), 6.25 − 6.10 (br, 1H), 5.51 (m, J = 8.5 Hz, 1H), 4.22 (dd, J = 7.4 Hz, 1H), 4.06 (d, J = 8.0 Hz, 2H), 3.93 (dd, J = 7.6 Hz, 1H), 3.85 (d, J = 10.9 Hz, 1H), 3.75 (d, J = 12.6 Hz, 1H), 3.62 (d, J = 10.5 Hz, 1H), 3.37 (d, J = 12.6 Hz, 1H), 3.01 (d, J = 6.1 Hz, 2H), 2.65 − 2.56 (m, 1H), 2.37 (s, 4H), 2.14 (d, J = 2.2 Hz, 6H), 2.07 (d, J = 22.6 Hz, 2H), 1.47 (s, J = 3.9 Hz, 1H). | 604.25 |
| 1-(4-(3-((5-(5- chlorobenzo[d] [1,3]dioxol-4- yl)-7-methyl- 7H-pyrrolo[2,3- d]pyrimidin-6- yl)ethynyl) azetidin-1- yl)piperidin-1- yl)prop-2-en-1- one | 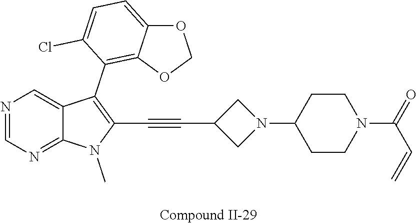 [SMILES: C=CC(=O)N1CCC(N2CC(C#Cc3c(-c4c(Cl)ccc5c4OCO5)c4cncnc4n3C)C2)CC1] | ¹H NMR (400 MHz, DMSO-d₆) δ 8.91 (s, 1H), 8.86 (s, 1H), 7.11 (d, J = 8.4 Hz, 1H), 7.03 (d, J = 8.3 Hz, 1H), 6.79 (dd, J = 16.7, 10.5 Hz, 1H), 6.05 (td, J = 16.7, 1.8 Hz, 3H), 5.64 (dd, J = 10.5, 2.5 Hz, 1H), 3.96 (s, 1H), 3.82 (d, J = 13.5 Hz, 4H), 3.53 (dt, J = 30.9, 7.5 Hz, 3H), 3.19 (t, J = 11.6 Hz, 1H), 3.08 − 2.95 (m, 3H), 2.32 − 2.23 (m, 1H), 1.61 (s, 2H), 1.07 (s, 2H). | 504.15 |
| 1-(4-(3-((5-(2- methoxy-4- phenoxyphenyl)- 7-methyl-7H- pyrrolo[2,3- d]pyrimidin-6- yl)ethynyl) azetidin-1- yl)piperidin-1- yl)prop-2-en-1- one | 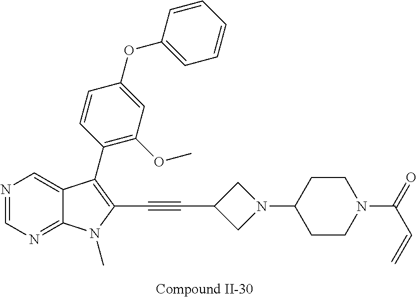 [SMILES: C=CC(=O)N1CCC(N2CC(C#Cc3c(-c4ccc(Oc5ccccc5)cc4OC)c4cncnc4n3C)C2)CC1] | ¹H NMR (400 MHz, DMSO-d₆) δ 8.84 (d, J = 12.8 Hz, 2H), 7.54 (d, J = 8.3 Hz, 1H), 7.49 − 7.40 (m, 2H), 7.25 − 7.12 (m, 3H), 6.91 (d, J = 2.3 Hz, 1H), 6.78 (dd, J = 16.7, 10.5 Hz, 1H), 6.61 (dd, J = 8.4, 2.4 Hz, 1H), 6.07 (dd, J = 16.7, 2.5 Hz, 1H), 5.65 (dd, J = 10.4, 2.5 Hz, 1H), 3.95 (s, 1H), 3.80 (s, 7H), 3.57 (t, J = 6.9 Hz, 2H), 3.48 (p, J = 7.2 Hz, 1H), 3.16 (t, J = 11.4 Hz, 1H), 3.05 (t, J = 6.6 Hz, 2H), 2.98 (t, J = 11.2 Hz, 1H), 2.26 (dt, J = 8.6, 4.6 Hz, 1H), 1.59 (s, 2H), 1.07 (s, 2H). | 548.3 |
| 1-(4-(3-((7- methyl-5-(4- phenoxyphenyl)- 7H- pyrrolo[2,3- d]pyrimidin-6- yl)ethynyl) azetidin-1- yl)piperidin-1- yl)prop-2-en-1- one | 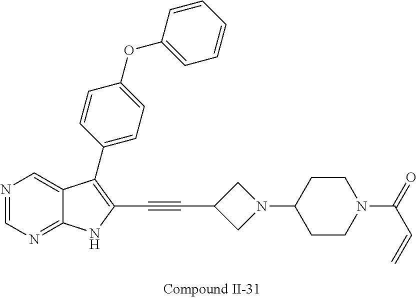 [SMILES: C=CC(=O)N1CCC(N2CC(C#Cc3[nH]c4ncncc4c3-c3ccc(Oc4ccccc4)cc3)C2)CC1] | ¹H NMR (400 MHz, DMSO-d₆) δ 9.19 (s, 1H), 8.91 (s, 1H), 7.88 (d, J = 8.5 Hz, 2H), 7.45 (t, J = 7.9 Hz, 2H), 7.25 − 7.09 (m, 5H), 6.78 (dd, J = 16.7, 10.5 Hz, 1H), 6.07 (dd, J = 16.7, 2.5 Hz, 1H), 5.65 (dd, J = 10.4, 2.5 Hz, 1H), 3.99 (d, J = 13.2 Hz, 1H), 3.84-3.81 (m, 4H), 3.58 (dq, J = 14.5, 7.5 Hz, 3H), 3.14 (q, J = 7.0, 5.9 Hz, 3H), 2.98 (t, J = 11.4 Hz, 1H), 2.29 (tt, J = 8.4, 3.8 Hz, 1H), 1.62 (s, 2H), 1.09 (d, J = 12.8 Hz, 2H). | 518.25 |
| 1-(4-(3-((4- amino-5-(3- (difluoromethoxy)- 4- phenoxyphenyl)- 7-methyl-7H- pyrrolo[2,3- d]pyrimidin-6- yl)ethynyl) azetidin-1- yl)piperidin-1- yl)prop-2-en-1- one | 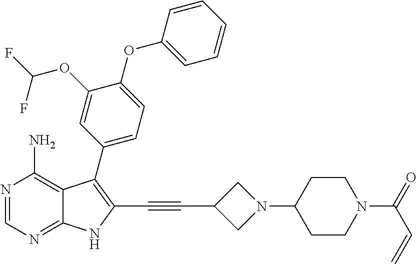 [SMILES: C=CC(=O)N1CCC(N2CC(C#Cc3[nH]c4ncnc(N)c4c3-c3ccc(Oc4ccccc4)c(OC(F)F)c3)C2)CC1] | ¹H NMR (400 MHz, DMSO-d₆) δ 8.20 (s, 1H), 7.47 (d, J = 2.2 Hz, 1H), 7.45 − 7.36 (m, 3H), 7.25 − 6.99 (m, 5H), 6.78 (dd, J = 16.7, 10.5 Hz, 1H), 6.38 (s, 2H), 6.07 (dd, J = 16.7, 2.5 Hz, 1H), 5.64 (dd, J = 10.5, 2.5 Hz, 1H), 3.98 (d, J = 12.8 Hz, 1H), 3.81 (d, J = 13.1 Hz, 1H), 3.73 (s, 3H), 3.56 (t, J = 6.9 Hz, 2H), 3.44 (p, J = 7.6 Hz, 1H), 3.20 − 3.09 (m, 1H), 3.06 − 2.99 (m, 2H), 2.95 (t, J = 11.3 Hz, 1H), 2.25 (s, 1H), 1.59 (s, 2H), 1.05 (s, 2H). | 599.3 |
| | Compound II-35 | | |
| 1-(4-(3-((4- amino-5-(3- ethoxy-4- phenoxyphenyl)- 7-methyl-7H- pyrrolo[2,3- d]pyrimidin-6- yl)ethynyl) azetidin-1- yl)piperidin-1- yl)prop-2-en-1- one | 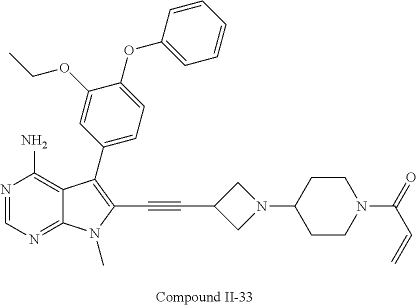 [SMILES: C=CC(=O)N1CCC(N2CC(C#Cc3c(-c4ccc(Oc5ccccc5)c(OCC)c4)c4c(N)ncnc4n3C)C2)CC1] | ¹H NMR (400 MHz, Chloroform-d) δ 8.37 (s, 1H), 7.35 (ddd, J = 8.5, 5.6, 2.0 Hz, 2H), 7.24 − 7.19 (m, 1H), 7.15 − 7.07 (m, 3H), 7.06 − 7.01 (m, 2H), 6.58 (dd, J = 16.8, 10.6 Hz, 1H), 6.27 (dd, J = 16.8, 2.0 Hz, 1H), 5.68 (dd, J = 10.6, 2.0 Hz, 1H), 5.25 (s, 2H), 4.25 (d, J = 13.3 Hz, 1H), 4.13 (q, J = 7.0 Hz, 2H), 3.86 (s, 4H), 3.69 (t, J = 6.9 Hz, 2H), 3.48 (p, J = 7.8 Hz, 1H), 3.18 (t, J = 11.7 Hz, 1H), 3.08 (t, J = 7.2 Hz, 3H), 2.30 (tt, J = 8.6, 3.5 Hz, 1H), 1.70 (dd, J = 13.7, 5.3 Hz, 2H), 1.35 (q, J = 9.7, 8.3 Hz, 3H), 1.27 (tq, J = 9.5, 4.7, 3.8 Hz, 2H). | 577.3 |
| 1-(4-(3-((4- amino-5-(2- ethoxy-4- phenoxyphenyl)- 7-methyl-7H- pyrrolo[2,3- d]pyrimidin-6- yl)ethynyl) azetidin-1- yl)piperidin-1- yl)prop-2-en-1- one | 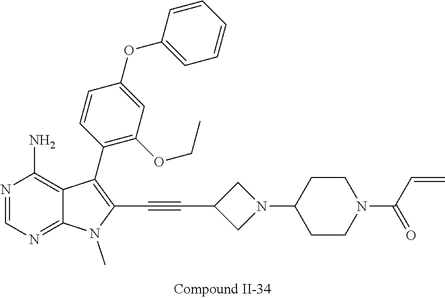 [SMILES: C=CC(=O)N1CCC(N2CC(C#Cc3c(-c4ccc(Oc5ccccc5)cc4OCC)c4c(N)ncnc4n3C)C2)CC1] | ¹H NMR (400 MHz, DMSO-d₆) δ 8.14 (s, 1H), 7.43 (t, J = 7.9 Hz, 2H), 7.30 (d, J = 8.3 Hz, 1H), 7.18 (t, J = 7.4 Hz, 1H), 7.13 (d, J = 8.0 Hz, 2H), 6.84 (d, J = 2.3 Hz, 1H), 6.78 (dd, J = 16.7, 10.5 Hz, 1H), 6.60 (dd, J = 8.4, 2.3 Hz, 1H), 6.07 (dd, J = 16.7, 2.5 Hz, 1H), 6.00 (s, 2H), 5.64 (dd, J = 10.4, 2.4 Hz, 1H), 4.00 (tt, J = 14.6, 6.5 Hz, 3H), 3.80 (d, J = 13.6 Hz, 1H), 3.70 (s, 3H), 3.53 (d, J = 8.7 Hz, 2H), 3.38 (t, J = 7.6 Hz, 1H), 3.16 (s, 1H), 2.94 (dt, J = 13.7, 6.9 Hz, 3H), 2.26 − 2.19 (m, 1H), 1.58 (s, 2H), 1.18 (t, J = 6.9 Hz, 3H), 1.04 (d, J = 14.8 Hz, 2H). | 577.4 |
| 1-(4-(3-((4- amino-5-(2- (difluoromethoxy)- 4- phenoxyphenyl)- 7-methyl-7H- pyrrolo[2,3- d]pyrimidin-6- yl)ethynyl) azetidin-1- yl)piperidin-1- yl)prop-2-en-1- one | 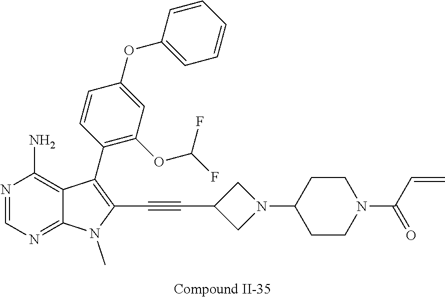 [SMILES: C=CC(=O)N1CCC(N2CC(C#Cc3c(-c4ccc(Oc5ccccc5)cc4OC(F)F)c4c(N)ncnc4n3C)C2)CC1] | ¹H NMR (400 MHz, Methanol-d₄) δ 8.18 (s, 1H), 7.46 (dt, J = 7.9, 3.6 Hz, 3H), 7.24 (t, J = 7.5 Hz, 1H), 7.19 − 7.13 (m, 2H), 7.01 − 6.92 (m, 2H), 6.82 − 6.54 (m, 2H), 6.18 (dd, J = 16.8, 2.0 Hz, 1H), 5.74 (dd, J = 10.6, 2.0 Hz, 1H), 4.37 (d, J = 13.2 Hz, 1H), 4.03 (d, J = 14.1 Hz, 1H), 3.83 (s, 3H), 3.67 (t, J = 7.4 Hz, 2H), 3.48 (p, J = 7.6 Hz, 1H), 3.18 (q, J = 9.4, 7.2 Hz, 3H), 2.91 (t, J = 12.1 Hz, 1H), 2.39 (s, 1H), 1.79 (d, J = 12.8 Hz, 2H), 1.17 (t, J = 11.1 Hz, 2H). | 599.3 |
| 1-[4-(3-[2-[4- amino-5-(5- chloro-2H-1,3- benzodioxol-4- yl)-7-methyl- 7H-pyrrolo[2,3- d]pyrimidin-6- yl]ethynyl] azetidin-1- yl)piperidin-1- yl]prop-2-en-1- one | 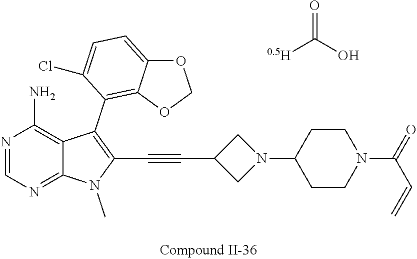 | ¹H NMR (400 MHz, DMSO-d₆) δ 8.22 (s, 0.5H), 8.17 (s, 1H), 7.07 (d, J = 8.4 Hz, 1H), 7.00 (d, J = 8.4 Hz, 1H), 6.79 (dd, J = 16.7, 10.5 Hz, 1H), 6.28 − 6.05 (m, 4.8H), 5.65 (dd, J = 10.5, 2.5 Hz, 1H), 3.97 (d, J = 13.3 Hz, 1H), 3.80 (s, 1H), 3.72 (s, 3H), 3.53 (t, J = 7.0 Hz, 2H), 3.42-3.35 (m, J = 7.5 Hz, 1H), 3.18 (t, 1H), 3.10- 2.92 (m, J = 17.6, 11.1 Hz, 3H), 2.23 (s, 1H), 1.59 (m, 2H), 1.05 (m, 2H). | 519.15 |
| 1-(4-(3-((4- amino-5- (benzo[d][1,3] dioxol-4-yl)-7- methyl-7H- pyrrolo[2,3- d]pyrimidin-6- yl)ethynyl) azetidin-1- yl)piperidin-1- yl)prop-2-en-1- one | 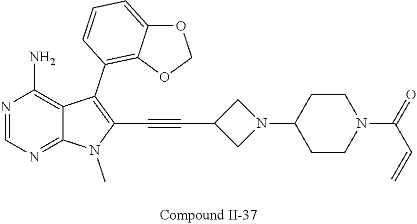 [SMILES: C=CC(=O)N1CCC(N2CC(C#Cc3c(-c4cccc5c4OCO5)c4c(N)ncnc4n3C)C2)CC1] | ¹H NMR (400 MHz, Chloroform-d) δ 8.35 (s, 1H), 7.04 − 6.99 (m, 2H), 6.97 − 6.89 (m, 1H), 6.58 (dd, J = 16.9, 10.6 Hz, 1H), 6.27 (dd, J = 16.9, 1.9 Hz, 1H), 6.04 (s, 2H), 5.69 (dd, J = 10.9, 1.9 Hz, 1H), 5.47 (m, 1H), 4.28 (s, 1H), 3.86 (s, 3H), 3.75 (d, J = 7.5 Hz, 2H), 3.52 (q, J = 7.6 Hz, 1H), 3.20 (s, 1H), 3.14 (d, J = 7.4 Hz, 3H), 2.38 (s, 1H), 1.73 (d, J = 12.9 Hz, 2H), 1.33 (dd, J = 9.4, 4.5 Hz, 2H). | 485.25 |
| 1-((1R,5S,6s)-6- ((4-amino-5-(5- chlorobenzo[d] [1,3]dioxol-4- yl)-7-methyl- 7H-pyrrolo[2,3- d]pyrimidin-6- yl)ethynyl)-3- aza- bicyclo[3.1.0] hexan-3-yl)prop-2- en-1-one | 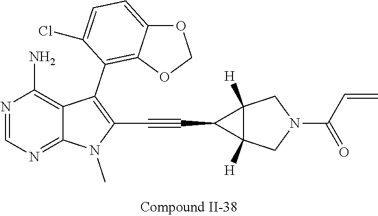 [SMILES: C=CC(=O)N1C[C@@H]2[C@@H](C#Cc3c(-c4c(Cl)ccc5c4OCO5)c4c(N)ncnc4n3C)[C@@H]2C1] | ¹H NMR (400 MHz, DMSO-d₆) δ 8.16 (s, 1H), 7.08 (d, J = 8.3 Hz, 1H), 7.00 (d, J = 8.4 Hz, 1H), 6.48 (dd, J = 16.9, 10.4 Hz, 1H), 6.11 (d, J = 2.2 Hz, 1H), 6.06 (d, J = 12.3 Hz, 3H), 5.66 − 5.63 (m, 1H), 3.87 (d, J = 10.8 Hz, 1H), 3.74 (d, J = 12.5 Hz, 1H), 3.70 (s, 3H), 3.62 (dd, J = 11.1, 4.2 Hz, 1H), 3.38 (dd, J = 12.6, 4.2 Hz, 1H), 2.10 − 1.98 (m, 2H), 1.47 (d, J = 4.0 Hz, 1H). | 462.2 |
| N-(2- ((1R,5S,6s)-6- ((4-amino-5-(5- chlorobenzo[d] [1,3]dioxol-4- yl)-7-methyl- 7H-pyrrolo[2,3- d]pyrimidin-6- yl)ethynyl)-3- aza- bicyclo[3.1.0] hexan-3-yl)-2- oxoethyl) acrylamide | 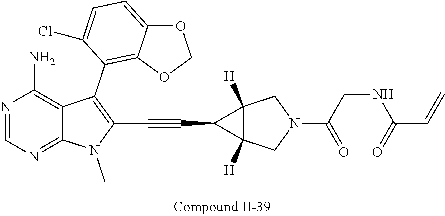 [SMILES: C=CC(=O)NCC(=O)N1C[C@@H]2[C@@H](C#Cc3c(-c4c(Cl)ccc5c4OCO5)c4c(N)ncnc4n3C)[C@@H]2C1] | ¹H NMR (400 MHz, Chloroform-d) δ 8.35 (s, 1H), 7.07 (d, J = 8.3 Hz, 1H), 6.87 (d, J = 8.3 Hz, 1H), 6.62 (s, 1H), 6.33 (dd, J = 17.0, 1.5 Hz, 1H), 6.19 (dd, J = 17.0, 10.2 Hz, 1H), 6.04 − 6.03 (m, 2H), 5.70 (dd, J = 10.2, 1.6 Hz, 1H), 5.15 (s, 2H), 4.04 (t, J = 3.9 Hz, 2H), 3.97 (dd, J = 12.3, 4.1 Hz, 1H), 3.85 (s, 3H), 3.70 (dd, J = 10.6, 3.8 Hz, 1H), 3.65 − 3.58 (m, 1H), 3.54 (dt, J = 12.4, 3.6 Hz, 1H), 2.06 (t, J = 4.4 Hz, 1H), 2.01 (tq, J = 7.4, 3.5 Hz, 1H), 1.36 (t, J = 3.4 Hz, 1H). | 519.2 |
| 1-[(1R,5S,6S)- 6-[2-[4-amino- 5-(2-methoxy-4- phenoxyphenyl)- 7-(oxolan-3- yl)-7H- pyrrolo[2,3- d]pyrimidin-6- yl]ethynyl]-3- azabicyclo[3.1.0] hexan-3- yl]prop-2-en-1- one | 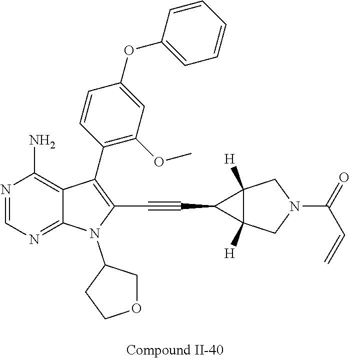 [SMILES: C=CC(=O)N1C[C@@H]2[C@@H](C#Cc3c(-c4ccc(Oc5ccccc5)cc4OC)c4c(N)ncnc4n3C3CCOC3)[C@@H]2C1] | ¹H NMR (400 MHz, Chloroform-d) δ 8.29 (s, 1H), 7.43 (t, J = 7.7 Hz, 2H), 7.32 (d, 1H), 7.21 (t, J = 7.5 Hz, 1H), 7.14 (d, J = 7.9 Hz, 2H), 6.76 (s, 1H), 6.66 (d, J = 8.3 Hz, 1H), 6.40 − 6.27 (m, 2H), 5.76 − 5.68 (m, 1H), 5.62 (t, J = 8.2 Hz, 1H), 5.43 (s, 1H), 4.42 − 4.22 (m, 2H), 4.17 (t, J = 8.8 Hz, 1H), 4.02 (d, J = 12.5 Hz, 2H), 3.84 (d, J = 10.7 Hz, 1H), 3.78 (s, 3H), 3.69 (d, J = 10.7 Hz, 1H), 3.53 (d, J = 12.8 Hz, 1H), 2.74 (m, J = 7.5 Hz, 1H), 2.44 (m, 1H), 1.99 (m, J = 3.7 Hz, 1H), 1.35 (s, 1H). | 562.20 |
| 1-((1R,5S,6s)-6- ((4-amino-5-(2- methoxy-4- phenoxyphenyl)- 7-methyl-7H- pyrrolo[2,3- d]pyrimidin-6- yl)ethynyl)-3- azabicyclo[3.1.0] hexan-3- yl)prop-2-en- 1-one | 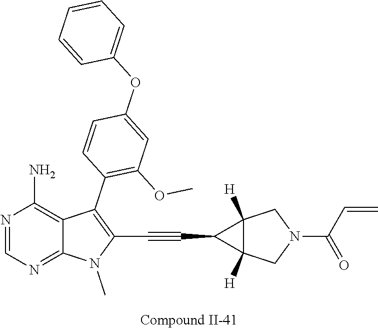 [SMILES: C=CC(=O)N1C[C@@H]2[C@@H](C#Cc3c(-c4ccc(Oc5ccccc5)cc4OC)c4c(N)ncnc4n3C)[C@@H]2C1] | ¹H NMR (400 MHz, Chloroform-d) δ 8.28 (s, 1H), 7.43 (t, J = 7.8 Hz, 2H), 7.31 (s, 1H), 7.20 (t, J = 7.5 Hz, 1H), 7.14 (d, J = 8.0 Hz, 2H), 6.75 (s, 1H), 6.66 (d, J = 8.3 Hz, 1H), 6.43 − 6.29 (m, 2H), 5.71 (dd, J = 8.6, 3.7 Hz, 1H), 5.66 (s, 1H), 4.02 (d, J = 12.8 Hz, 1H), 3.80 (d, J = 15.3 Hz, 7H), 3.69 (dd, J = 10.7, 3.6 Hz, 1H), 3.53 (d, J = 13.5 Hz, 1H), 1.98 (s, 2H), 1.37 (s, 1H), 1.28 (s, 1H). | 506.30 |
| 1-((1R,5S,6s)-6- ((4-amino-5-(2- methoxy-4-((6- methylpyridin-2- yl)oxy)phenyl)- 7-methyl-7H- pyrrolo[2,3- d]pyrimidin-6- yl)ethynyl)-3- azabicyclo[3.1.0] hexan-3- yl)prop-2-en- 1-one | 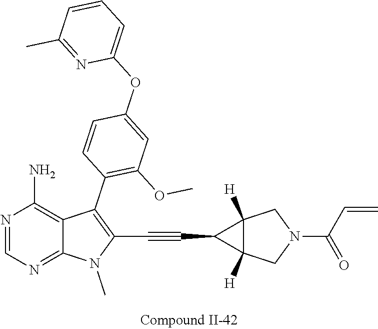 [SMILES: C=CC(=O)N1C[C@@H]2[C@@H](C#Cc3c(-c4ccc(Oc5cccc(C)n5)cc4OC)c4c(N)ncnc4n3C)[C@@H]2C1] | ¹H NMR (400 MHz, DMSO-d₆) δ 8.14 (s, 1H), 7.77 (t, J = 7.8 Hz, 1H), 7.29 (d, J = 8.3 Hz, 1H), 7.06 (d, J = 7.4 Hz, 1H), 6.96 (d, J = 2.3 Hz, 1H), 6.86 (d, J = 8.2 Hz, 1H), 6.78 (dd, J = 8.1, 2.2 Hz, 1H), 6.49 (dd, J = 16.8, 10.3 Hz, 1H), 6.10 (dd, J = 16.8, 2.4 Hz, 1H), 5.98 (br, 1H), 5.65 (dd, J = 10.3, 2.4 Hz, 1H), 3.87 (d, J = 10.8 Hz, 1H), 3.73 (s, 4H), 3.69 (s, 3H), 3.62 (dd, J = 10.7, 4.1 Hz, 1H), 3.38 (dd, J = 12.5, 4.2 Hz, 1H), 2.39 (s, 3H), 2.05 (d, J = 7.7 Hz, 1H), 2.00 (d, J = 9.8 Hz, 1H), 1.45 (t, J = 3.4 Hz, 1H). | 521.3 |
| (E)-1- ((1R,5S,6s)-6- ((4-amino-5-(4- (3- fluorophenoxy) phenyl)-7- methyl-7H- pyrrolo[2,3- d]pyrimidin-6- yl)ethynyl)-3- azabicyclo[3.1.0] hexan-3-yl)-4- (dimethylamino) but-2-en-1-one | 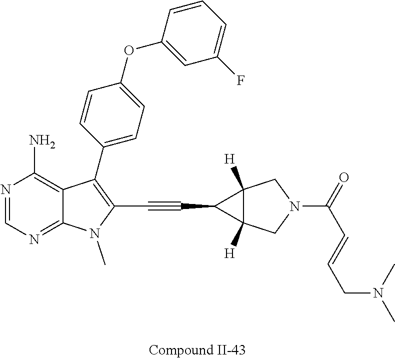 [SMILES: CN(C)C/C=C/C(=O)N1C[C@@H]2[C@@H](C#Cc3c(-c4ccc(Oc5cccc(F)c5)cc4)c4c(N)ncnc4n3C)[C@@H]2C1] | ¹H NMR (400 MHz, DMSO-d₆) δ 8.18 (s, 1H), 7.55 − 7.48 (m, 2H), 7.45 (q, J = 7.7 Hz, 1H), 7.23 − 7.16 (m, 2H), 7.06 − 6.94 (m, 3H), 6.59 (dt, J = 15.3, 6.2 Hz, 1H), 6.28 (d, J = 15.4 Hz, 1H), 3.85 (d, J = 10.7 Hz, 1H), 3.74 (d, J = 12.5 Hz, 1H), 3.70 (s, 3H), 3.63 (dd, J = 10.8, 4.2 Hz, 1H), 3.38 (dd, J = 12.6, 4.3 Hz, 1H), 3.01 (d, J = 6.1 Hz, 2H), 2.14 (s, 6H), 2.13 − 2.07 (m, 1H), 2.11 − 1.99 (m, 1H), 1.48 (t, J = 3.5 Hz, 1H). | 551.4 |
| (E)-1- ((1R,5S,6s)-6- ((4-amino-5-(2- methoxy-4- phenoxyphenyl)- 7-methyl-7H- pyrrolo[2,3- d]pyrimidin-6- yl)ethynyl)-3- azabicyclo[3.1.0] hexan-3-yl)-4- (dimethylamino) but-2-en-1-one formate | 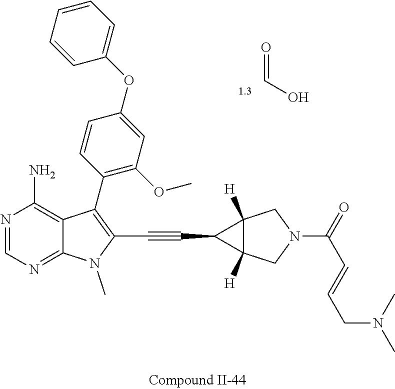 [SMILES: COc1cc(Oc2ccccc2)ccc1-c1c(C#C[C@@H]2[C@H]3CN(C(=O)/C=C/CN(C)C)C[C@@H]23)n(C)c2ncnc(N)c12 ; O=CO] | ¹H NMR (400 MHz, Methanol-d₄₎ δ 8.37 (s, 1H), 8.15 (s, 1H), 7.43 (t, J = 7.7 Hz, 2H), 7.31 (d, J = 8.3 Hz, 1H), 7.19 (t, J = 7.4 Hz, 1H), 7.14 (d, J = 8.0 Hz, 2H), 6.83 (s, 1H), 6.73 (dt, J = 14.4, 7.0 Hz, 1H), 6.63 (t, J = 13.6 Hz, 2H), 3.92 (t, J = 12.6 Hz, 2H), 3.77 (d, J = 6.2 Hz, 9H), 3.58 − 3.50 (m, 1H), 2.78 (s, 6H), 2.06 (d, J = 8.2 Hz, 2H), 1.43 (s, 1H). | 563.35 |
| (E)-1- ((1R,5S,6s)-6- ((4-amino-5-(4- (3- fluorophenoxy)- 2- methoxyphenyl)- 7-methyl-7H- pyrrolo[2,3- d]pyrimidin-6- yl)ethynyl)-3- azabicyclo[3.1.0] hexan-3-yl)-4- (dimethylamino) but-2-en-1-one | 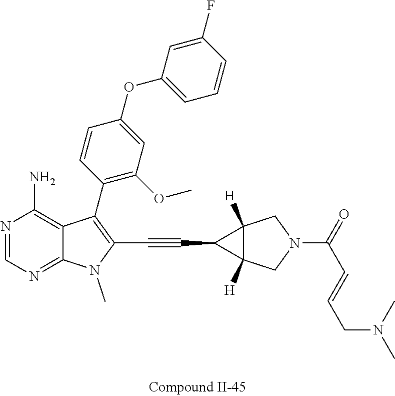 [SMILES: COc1cc(Oc2cccc(F)c2)ccc1-c1c(C#C[C@@H]2[C@H]3CN(C(=O)/C=C/CN(C)C)C[C@@H]23)n(C)c2ncnc(N)c12] | ¹H NMR (400 MHz, DMSO-d₆) δ 8.14 (s, 1H), 7.46 (q, J = 7.7 Hz, 1H), 7.28 (d, J = 8.2 Hz, 1H), 7.07 − 6.96 (m, 3H), 6.92 (d, J = 2.4 Hz, 1H), 6.69 (dd, J = 8.3, 2.3 Hz, 1H), 6.58 (dt, J = 15.2, 6.2 Hz, 1H), 6.32 − 6.23 (m, 1H), 6.02 (s, 2H), 3.83 (d, J = 10.7 Hz, 1H), 3.76 − 3.66 (m, 7H), 3.61 (dd, J = 10.8, 4.2 Hz, 1H), 3.41 − 3.33 (m, 1H), 3.04 − 2.97 (m, 2H), 2.14 (s, 6H), 2.07 − 1.90 (m, 2H), 1.44 (t, J = 3.4 Hz, 1H). | 581.3 |
| (E)-1- ((1R,5S,6s)-6- ((4-amino-7- methyl-5-(4-((6- methylpyridin-2- yl)oxy)phenyl)- 7H-pyrrolo[2,3- d]pyrimidin-6- yl)ethynyl)-3- azabicyclo[3.1.0] hexan-3-yl)-4- (methylamino) but-2-en-1-one | 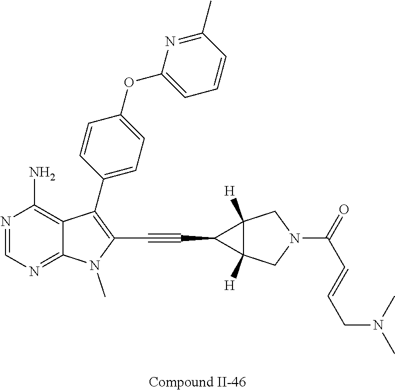 [SMILES: Cc1cccc(Oc2ccc(-c3c(C#C[C@@H]4[C@H]5CN(C(=O)/C=C/CN(C)C)C[C@@H]45)n(C)c4ncnc(N)c34)cc2)n1] | ¹H NMR (400 MHz, DMSO-d₆) δ 8.19 (s, 1H), 7.77 (t, J = 7.7 Hz, 1H), 7.51 (d, J = 8.4 Hz, 2H), 7.25 (d, J = 8.4 Hz, 2H), 7.05 (d, J = 7.4 Hz, 1H), 6.86 (d, J = 8.2 Hz, 1H), 6.64 (dt, J = 15.2, 5.4 Hz, 1H), 6.26 (d, J = 15.1 Hz, 1H), 3.85 (d, J = 10.8 Hz, 1H), 3.72 (d, J = 8.2 Hz, 4H), 3.62 (dd, J = 10.7, 4.2 Hz, 1H), 3.42 − 3.36 (m, 1H), 3.24 (d, J = 5.5 Hz, 2H), 2.38 (s, 3H), 2.27 (s, 3H), 2.12 (dt, J = 7.6, 3.5 Hz, 1H), 2.06 (s, 1H), 1.47 (t, J = 3.4 Hz, 1H). | 533.64 |
| (E)-1- ((1R,5S,6s)-6- ((4-amino-5-(3- methoxy-4- (pyridin-2- yloxy)phenyl)- 7-methyl-7H- pyrrolo[2,3- d]pyrimidin-6- yl)ethynyl)-3- azabicyclo[3.1.0] hexan-3-yl)-4- (3- fluoropyrrolidin- 1-yl)but-2-en- 1-one | 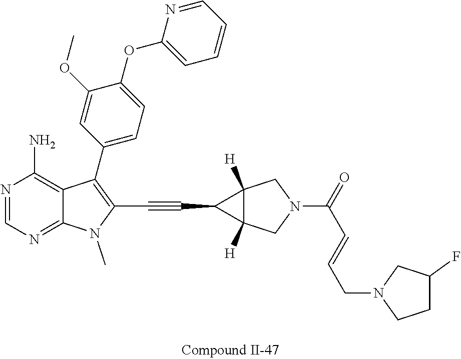 [SMILES: COc1cc(-c2c(C#C[C@@H]3[C@H]4CN(C(=O)/C=C/CN5CCC(F)C5)C[C@@H]34)n(C)c3ncnc(N)c23)ccc1Oc1ccccn1] | ¹H NMR (400 MHz, DMSO-d₆) δ 8.20 (s, 1H), 8.13 (dd, J = 5.1, 1.9 Hz, 1H), 7.84 (ddd, J = 8.7, 7.2, 2.0 Hz, 1H), 7.26 (d, J = 8.2 Hz, 2H), 7.14 − 7.06 (m, 2H), 7.04 (d, J = 8.3 Hz, 1H), 6.67 − 6.53 (m, 1H), 6.31 (dd, J = 15.3, 1.7 Hz, 1H), 5.19 (ddd, J = 55.9, 6.5, 4.8 Hz, 1H), 3.86 (dd, J = 10.7, 2.7 Hz, 1H), 3.79 − 3.69 (m, 7H), 3.64 (dd, J = 10.7, 4.3 Hz, 1H), 3.39 (dd, J = 12.6, 4.4 Hz, 1H), 3.24 − 3.18 (m, 2H), 2.88 − 2.70 (m, 2H), 2.70 − 2.54 (m, 2H), 2.34 (d, J = 15.1 Hz, 1H), 2.20 − 2.02 (m, 3H), 1.87 (ddt, J = 28.6, 14.0, 6.8 Hz, 1H), 1.53 (t, J = 3.4 Hz, 1H). | 607.63 |
| 1-((1R,5S,6s)-6- ((4-amino-5-(2- chloro-4- phenoxyphenyl)- 7-methyl-7H- pyrrolo[2,3- d]pyrimidin-6- yl)ethynyl)-3- azabicyclo[3.1.0] hexan-3- yl)prop-2-en-1- one | 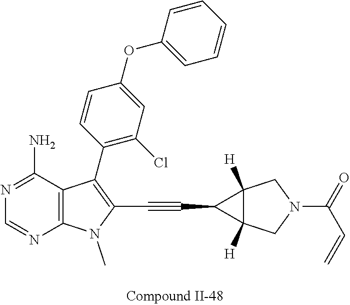 [SMILES: C=CC(=O)N1C[C@@H]2[C@@H](C#Cc3c(-c4ccc(Oc5ccccc5)cc4Cl)c4c(N)ncnc4n3C)[C@@H]2C1] | ¹H NMR (400 MHz, Methanol-d₄) δ 8.18 (s, 1H), 7.47 (dd, J = 8.4, 7.4 Hz, 2H), 7.42 (d, J = 8.4 Hz, 1H), 7.25 (t, J = 7.5 Hz, 1H), 7.17 (dt, J = 4.4, 2.0 Hz, 3H), 7.05 (dd, J = 8.5, 2.5 Hz, 1H), 6.53 (dd, J = 16.8, 10.5 Hz, 1H), 6.25 (dd, J = 16.8, 2.0 Hz, 1H), 5.74 (dd, J = 10.4, 2.0 Hz, 1H), 3.92 (dd, J = 14.9, 11.9 Hz, 2H), 3.80 (s, 3H), 3.74 (dd, J = 11.0, 4.2 Hz, 1H), 3.57 − 3.49 (m, 1H), 2.06 (dh, J = 14.7, 3.7 Hz, 2H), 1.43 (t, J = 3.5 Hz, 1H). | 510.15 |
| 1-[(1R,5S,6R)- 6-[2-[4-amino- 7-(3- hydroxypropyl)- 5-[2-methoxy-4- [(6- methylpyridin-2- yl)oxy]phenyl]- 7H-pyrrolo[2,3- d]pyrimidin-6- yl]ethynyl]-3- azabicyclo[3.1.0] hexan-3- yl]prop-2-en- 1-one | 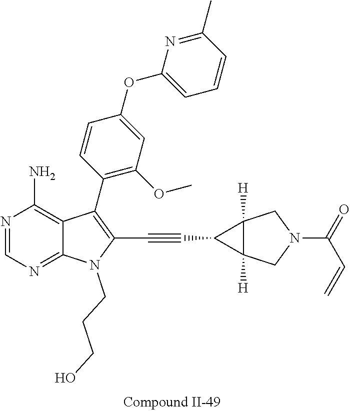 [SMILES: C=CC(=O)N1C[C@@H]2[C@@H](C#Cc3c(-c4ccc(Oc5cccc(C)n5)cc4OC)c4c(N)ncnc4n3CCCO)[C@@H]2C1] | ¹H NMR (400 MHz, DMSO-d₆) δ 8.13 (s, J = 1.4 Hz, 1H), 7.77 (t, J = 7.7 Hz, 1H), 7.30 (d, J = 8.3 Hz, 1H), 7.06 (d, J = 7.4 Hz, 1H), 6.97 (s, J = 2.1 Hz, 1H), 6.86 (d, J = 8.1 Hz, 1H), 6.77 (d, J = 8.3 Hz, 1H), 6.49 (dd, J = 16.7, 10.3 Hz, 1H), 6.10 (d, 1H), 6.05 − 5.75 (br, 1H), 5.65 (d, J = 10.3 Hz, 1H), 4.66 (t, J = 5.3 Hz, 1H), 4.23 (t, J = 7.3 Hz, 2H), 3.88 (d, J = 10.8 Hz, 1H), 3.73 (sd, J = 4.8 Hz, 4H), 3.61 (dd, J = 11.0, 4.2 Hz, 1H), 3.45 (q, J = 6.0 Hz, 2H), 3.37 (dd, 1H), 2.39 (s, 3H), 2.06 (m, J = 7.6, 3.9, 3.4 Hz, 1H), 2.00 (m, J = 9.7 Hz, 1H), 1.89 (p, J = 6.9 Hz, 2H), 1.44 (s, J = 3.7 Hz, 1H). | 565.35 |
| 1-((1R,5S,6s)-6- ((4-amino-5-(2- methoxy-4-((6- methylpyridin-2- yl)oxy)phenyl)-7- ((methylsulfonyl) methyl)-7H- pyrrolo[2,3- d]pyrimidin-6- yl)ethynyl)-3- azabicyclo[3.1.0] hexan-3- yl)prop-2-en-1- one | 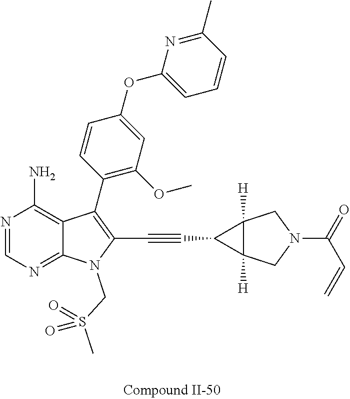 [SMILES: C=CC(=O)N1C[C@@H]2[C@@H](C#Cc3c(-c4ccc(Oc5cccc(C)n5)cc4OC)c4c(N)ncnc4n3CS(C)(=O)=O)[C@@H]2C1] | ¹H NMR (400 MHz, DMSO-d₆) δ 8.20 (s, 1H), 7.78 (t, J = 7.8 Hz, 1H), 7.33 (d, J = 8.3 Hz, 1H), 7.06 (d, J = 7.4 Hz, 1H), 6.98 (d, J = 2.2 Hz, 1H), 6.88 (d, J = 8.2 Hz, 1H), 6.79 (dd, J = 8.3, 2.2 Hz, 1H), 6.49 (dd, J = 16.8, 10.3 Hz, 1H), 6.25 (s, 2H), 6.09 (dd, J =16.8, 2.4 Hz, 1H), 5.68 − 5.55 (m, 3H), 3.87 (d, J = 10.8 Hz, 1H), 3.74 (d, J = 11.8 Hz, 4H), 3.62 (dd, J = 10.7, 4.2 Hz, 1H), 3.38 (dd, J = 12.6, 4.3 Hz, 1H), 3.14 (s, 3H), 2.39 (s, 3H), 2.05 (dtt, J = 21.5, 7.2, 3.9 Hz, 2H), 1.45 (t, J = 3.5 Hz, 1H). | 599.2 |
| 1-((1R,5S,6s)-6- ((4-amino-5-(3- methoxy-4-((6- methylpyridin- 2- yl)oxy)phenyl)- 7- ((methylsulfonyl) methyl)-7H- pyrrolo[2,3- d]pyrimidin-6- yl)ethynyl)-3- azabicyclo[3.1.0] hexan-3- yl)prop-2-en-1- one | 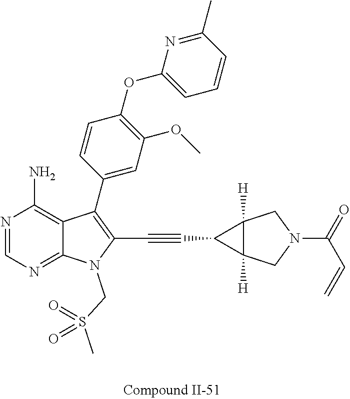 [SMILES: C=CC(=O)N1C[C@@H]2[C@@H](C#Cc3c(-c4ccc(Oc5cccc(C)n5)c(OC)c4)c4c(N)ncnc4n3CS(C)(=O)=O)[C@@H]2C1] | ¹H NMR (400 MHz, DMSO-d₆) δ 8.26 (s, 1H), 7.70 (t, J = 7.7 Hz, 1H), 7.30 − 7.23 (m, 2H), 7.12 (dd, J = 8.1, 2.0 Hz, 1H), 6.96 (d, J = 7.3 Hz, 1H), 6.75 (d, J = 8.2 Hz, 1H), 6.50 (dd, J = 16.7, 10.3 Hz, 1H), 6.10 (dd, J = 16.8, 2.4 Hz, 1H), 5.69 − 5.61 (m, 3H), 3.88 (d, J = 10.8 Hz, 1H), 3.75 (d, J = 10.6 Hz, 4H), 3.64 (dd, J = 10.9, 4.3 Hz, 1H), 3.40 (dd, J = 12.7, 4.3 Hz, 1H), 3.33 (s, 3H), 2.33 (s, 3H), 2.15 (dt, J = 7.5, 3.8 Hz, 1H), 2.08 (dt, J = 7.4, 3.8 Hz, 1H), 1.54 (t, J = 3.4 Hz, 1H). | 599.2 |
| 1-[(1R,5S,6R)- 6-[2-[4-amino- 7-(3- methanesulfonyl propyl)-5-[2- methoxy-4-[(6- methylpyridin- 2- yl)oxy]phenyl]- 7H-pyrrolo[2,3- d]pyrimidin-6- yl]ethynyl]-3- azabicyclo[3.1.0] hexan-3- yl]prop-2-en-1- one | 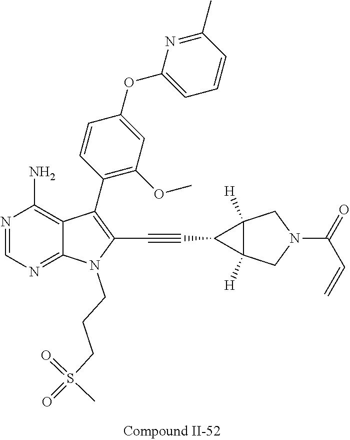 [SMILES: C=CC(=O)N1C[C@@H]2[C@@H](C#Cc3c(-c4ccc(Oc5cccc(C)n5)cc4OC)c4c(N)ncnc4n3CCCS(C)(=O)=O)[C@@H]2C1] | ¹H NMR (400 MHz, Methanol-d4) δ 8.23 (s, 1H), 7.78 (t, J = 7.8 Hz, 1H), 7.41 (d, J = 8.3 Hz, 1H), 7.07 (d, J = 7.4 Hz, 1H), 6.97 (s, J = 2.2 Hz, 1H), 6.89 (d, J = 8.2 Hz, 1H), 6.81 (d, J = 8.2 Hz, 1H), 6.53 (dd, J = 16.8, 10.4 Hz, 1H), 6.25 (d, J = 16.8 Hz, 1H), 5.74 (d, J = 10.3 Hz, 1H), 4.50 (t, J = 7.1 Hz, 2H), 3.92 (t, J = 13.0 Hz, 2H), 3.80 (s, 3H), 3.74 (dd, J = 10.9, 4.2 Hz, 1H), 3.52 (dd, J = 12.8, 4.2 Hz, 1H), 3.28 − 3.20 (m, 2H), 3.01 (s, 3H), 2.46 (s, 3H), 2.38 (t, J = 7.5 Hz, 2H), 2.18 − 2.06 (m, 2H), 1.44 (s, 1H). | 627.35 |
| 1-((3S,4R)-4-(3- ((4-amino-5-(4- chlorophenyl)- 7-isopropyl-7H- pyrrolo[2,3- d]pyrimidin-6- yl)ethynyl) azetidin- 1-yl)-3- hydroxypiperidi n-1-yl)prop-2- en-1-one | 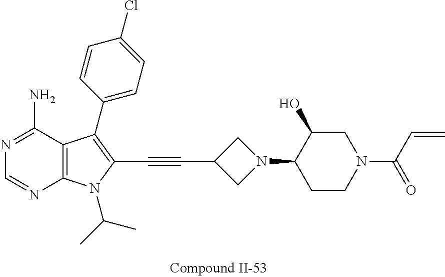 [SMILES: C=CC(=O)N1CC[C@@H](N2CC(C#Cc3c(-c4ccc(Cl)cc4)c4c(N)ncnc4n3C(C)C)C2)[C@@H](O)C1] | ¹H NMR (400 MHz, DMSO-d₆) δ 8.17 (s, 1H), 7.54 (q, J = 8.5 Hz, 4H), 6.84 − 6.67 (m, 1H), 6.05 (dd, J = 16.7, 2.6 Hz, 1H), 5.66 − 5.59 (m, 1H), 5.15 − 5.07 (m, 1H), 4.54 − 4.36 (m, 1H), 4.03 − 3.75 (m, 1H), 3.62 (ddd, J = 30.9, 17.7, 6.8 Hz, 4H), 3.42 (t, J = 7.3 Hz, 1H), 3.23 (d, J = 13.4 Hz, 1H), 3.07 (dt, J = 14.2, 6.8 Hz, 2H), 2.98 − 2.91 (m, 1H), 2.37 − 2.28 (m, 1H), 1.62 (d, J = 6.8 Hz, 6H), 1.52 −1.38 (m, 2H). | 519.3 |
| 1-((3S,4R)-4-(3- ((4-amino-5-(4- chlorophenyl)- 7-(2- methoxyethyl)- 7H-pyrrolo[2,3- d]pyrimidin-6- yl)ethynyl) azetidin- 1-yl)-3- hydroxypiperidin- 1-yl)prop-2- en-1-one | 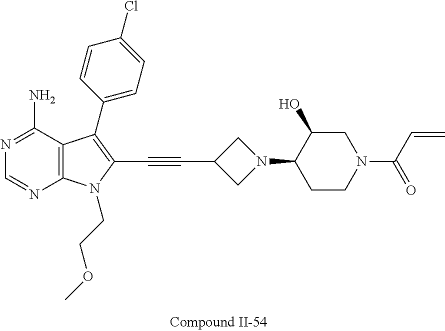 [SMILES: C=CC(=O)N1CC[C@@H](N2CC(C#Cc3c(-c4ccc(Cl)cc4)c4c(N)ncnc4n3CCOC)C2)[C@@H](O)C1] | ¹H NMR (400 MHz, DMSO-d₆) δ 8.19 (s, 1H), 7.54 (q, J = 8.3 Hz, 4H), 6.75 (ddd, J = 20.0, 16.8, 10.4 Hz, 1H), 6.23 (s, 1H), 6.05 (d, J = 16.7 Hz, 1H), 5.69 − 5.58 (m, 1H), 4.59 − 4.33 (m, 3H), 4.00 (d, J = 13.0 Hz, 1H), 3.86 − 3.66 (m, 3H), 3.61 (dt, J = 26.0, 7.3 Hz, 3H), 3.42 (p, J = 7.6 Hz, 1H), 3.22 (d, J = 13.3 Hz, 1H), 3.09 (dt, J = 13.9, 6.9 Hz, 2H), 2.99 − 2.88 (m, 1H), 2.42 − 2.25 (m, 1H), 1.59 − 1.33 (m, 2H). | 535.25 |
| 1-((3S,4R)-4-(3- ((4-amino-5-(4- chloro-3- methoxyphenyl)- 7-isopropyl- 7H-pyrrolo[2,3- d]pyrimidin-6- yl)ethynyl)azetidin- 1-yl)-3- hydroxypiperidin- 1-yl)prop-2- en-1-one | 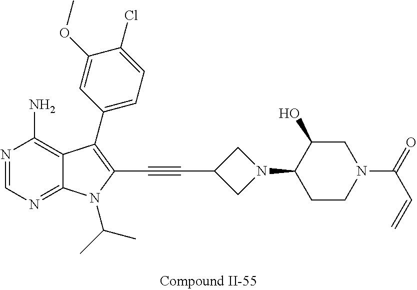 [SMILES: C=CC(=O)N1CC[C@@H](N2CC(C#Cc3c(-c4ccc(Cl)c(OC)c4)c4c(N)ncnc4n3C(C)C)C2)[C@@H](O)C1] | ¹H NMR (400 MHz, DMSO-d₆) δ 8.17 (s, J = 1.3 Hz, 1H), 7.53 (d, J = 8.1 Hz, 1H), 7.22 (s, 1H), 7.08 (d, J = 8.1 Hz, 1H), 6.73 (dd, J = 17.5, 10.8 Hz, 1H), 6.20 (br, 2H), 6.05 (d, J = 16.8 Hz, 1H), 5.63 (d, J = 10.6 Hz, 1H), 5.13 (d, J = 8.5 Hz, 1H), 4.54- 4.41 (m, 1H), 3.97 (s, 1H), 3.90 (s, 3H), 3.82 − 3.74 (m, 1H), 3.63 (d, J = 7.2 Hz, 1H), 3.58 (t, 2H), 3.49 − 3.40 (m, 1H), 3.24 (m, 1H), 3.06 (m, 2H), 2.50-2.27 (m, 2H), 1.63 (d, J = 6.9 Hz, 6H), 1.55-1.38 (m, 2H). | 549.35 |
| 1-((3S,4R)-4-(3- ((4-amino-5-(4- chloro-3- methoxyphenyl)- 7-(2- methoxyethyl)- 7H-pyrrolo[2,3- d]pyrimidin-6- yl)ethynyl)azetidin- 1-yl)-3- hydroxypiperidin- 1-yl)prop-2- en-1-one | 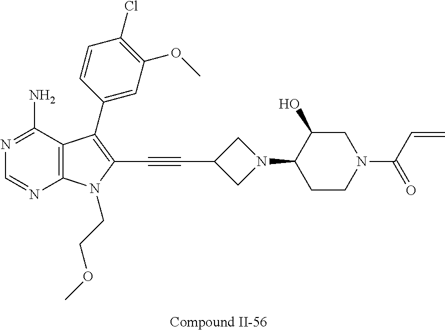 [SMILES: C=CC(=O)N1CC[C@@H](N2CC(C#Cc3c(-c4ccc(Cl)c(OC)c4)c4c(N)ncnc4n3CCOC)C2)[C@@H](O)C1] | ¹H NMR (400 MHz, Methanol-d₄) δ 8.25 (s, 1H), 7.54 (d, J = 8.1 Hz, 1H), 7.22 (d, J = 1.9 Hz, 1H), 7.16 (dd, J = 8.1, 1.9 Hz, 1H), 6.81 − 6.69 (m, 1H), 6.21 (dd, J = 16.8, 1.9 Hz, 1H), 5.82 − 5.72 (m, 1H), 4.73 (dd, J = 26.3, 14.1 Hz, 1H), 4.55 (t, J = 5.6 Hz, 2H), 4.47 (t, J = 9.4 Hz, 2H), 4.32 − 4.14 (m, 3H), 4.14 − 4.01 (m, 2H), 3.96 (s, 3H), 3.83 (t, J = 5.6 Hz, 2H), 3.51 (d, J = 12.6 Hz, 1H), 3.35 (s, 3H), 3.20 − 3.08 (m, 1H), 2.71 (t, J = 12.8 Hz, 1H), 1.86 (d, J = 11.4 Hz, 1H), 1.69 (pd, J = 12.3, 11.7, 4.2 Hz, 1H). | 565.20 |
| 1-((3S,4R)-4-(3- ((4-amino-7- isopropyl-5-(4- (oxetan-3- yloxy)phenyl)- 7H-pyrrolo[2,3- d]pyrimidin-6- yl)ethynyl) azetidin-1-yl)-3- hydroxypiperidin- 1-yl)prop-2- en-1-one | 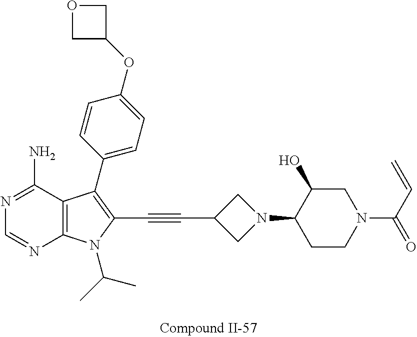 [SMILES: C=CC(=O)N1CC[C@@H](N2CC(C#Cc3c(-c4ccc(OC5COC5)cc4)c4c(N)ncnc4n3C(C)C)C2)[C@@H](O)C1] | ¹H NMR (400 MHz, DMSO-d₆) δ 8.15 (s, 1H), 7.47 − 7.40 (m, 2H), 6.93 (d, J = 8.3 Hz, 2H), 6.76 (ddd, J = 20.4, 16.7, 10.5 Hz, 1H), 6.11 − 6.07 (m, 2H), 5.63 (dd, J = 10.2, 3.0 Hz, 1H), 5.34 (p, J = 5.5 Hz, 1H), 5.13 − 5.03 (m, 1H), 4.97 (t, J = 6.6 Hz, 2H), 4.64 − 4.42 (m, 3H), 3.97 − 3.79 (m, 1H), 3.70 − 3.49 (m, 4H), 3.46 − 3.35 (m, 1H), 3.23 (d, J = 13.3 Hz, 1H), 3.06 (dt, J = 14.1, 6.8 Hz, 2H), 2.93 (dd, J = 13.5, 7.0 Hz, 1H), 2.36 − 2.28 (m, 1H), 1.62 (s, 6H), 1.52 − 1.38 (m, 2H). | 557.4 |
| 1-((3S,4R)-4-(3- ((4-amino-5-(4- chloro-3- (fluoromethyl) phenyl)-7- methyl-7H- pyrrolo[2,3- d]pyrimidin-6- yl)ethynyl) azetidin-1-yl)-3- hydroxypiperidin- 1-yl)prop-2- en-1-one | 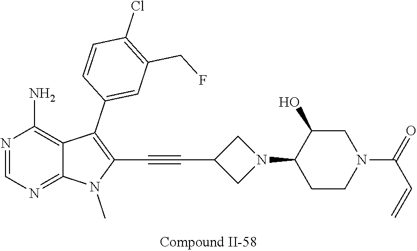 [SMILES: C=CC(=O)N1CC[C@@H](N2CC(C#Cc3c(-c4ccc(Cl)c(CF)c4)c4c(N)ncnc4n3C)C2)[C@@H](O)C1] | ¹H NMR (400 MHz, DMSO-d₆) δ 8.20 (s, 1H), 7.72 (s, 1H), 7.65 (d, J = 8.2 Hz, 1H), 7.53 (d, J = 8.3 Hz, 1H), 6.76 (ddd, J = 21.3, 16.7, 10.4 Hz, 1H), 6.29 (s, 1H), 6.05 (dd, J = 16.7, 2.5 Hz, 1H), 5.64 (d, J = 15.9 Hz, 2H), 5.54 (s, 1H), 4.53 − 4.41 (d, J = 3.1 Hz, 1H), 4.03 − 3.81 (dd, J = 13.3, 4.8 Hz, 1H), 3.73 (s, 3H), 3.66 (d, J = 6.3 Hz, 1H), 3.62 − 3.54 (m, 3H), 3.45 (q, J = 7.6 Hz, 1H), 3.19 (d, J = 13.5 Hz, 1H), 3.09 (dt, J = 13.8, 7.0 Hz, 2H), 2.92 − 2.84 (m, 1H), 2.37 − 2.29 (s, 1H), 1.51-1.38 (d, J = 5.8 Hz, 2H). | 523.2 |
| 1-((3S,4R)-4-(3- ((4-amino-5-(4- chloro-3- (hydroxymethyl) phenyl)-7- methyl-7H- pyrrolo[2,3- d]pyrimidin-6- yl)ethynyl)azetidin- 1-yl)-3- hydroxypiperidin- 1-yl)prop-2- en-1-one | 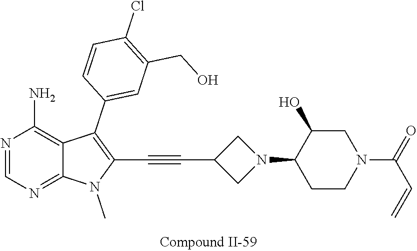 [SMILES: C=CC(=O)N1CC[C@@H](N2CC(C#Cc3c(-c4ccc(Cl)c(CO)c4)c4c(N)ncnc4n3C)C2)[C@@H](O)C1] | ¹H NMR (400 MHz, DMSO-d₆) δ 8.20 (s, 1H), 7.73 (d, J = 2.3 Hz, 1H), 7.53 (d, J = 8.2 Hz, 1H), 7.40 (d, J = 7.8 Hz, 1H), 6.76 (ddd, J = 21.7, 16.7, 10.5 Hz, 1H), 6.24 (s, 1H), 6.05 (dd, J = 16.8, 2.5 Hz, 1H), 5.63 (dt, J = 9.9, 3.7 Hz, 1H), 5.47 (t, J = 4.5 Hz, 1H), 4.64 (d, J = 5.3 Hz, 2H), 4.40 (d, J = 3.2 Hz, 1H), 4.05 − 3.77 (m, 1H), 3.73 (s, 3H), 3.65 (d, J = 6.2 Hz, 1H), 3.65 − 3.56 (m, 3H), 3.45 (q, J = 7.6 Hz, 1H), 3.18 (t, J = 13.4 Hz, 1H), 3.09 (t, J = 6.9 Hz, 2H), 2.92 − 2.84 (m, 1H), 2.31 − 2.28 (s, 1H), 1.38 (s, 2H). | 521.2 |
| 1-((3S,4R)-4-(3- ((4-amino-5-(4- chloro-3,5- dimethoxyphenyl)- 7-methyl-7H- pyrrolo[2,3- d]pyrimidin-6- yl)ethynyl) azetidin-1-yl)-3- hydroxypiperidin- 1-yl)prop-2- en-1-one | 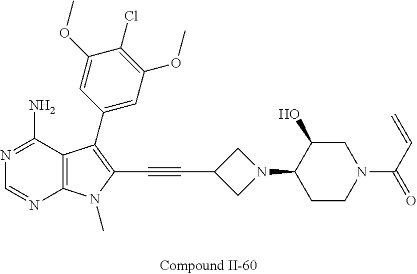 [SMILES: C=CC(=O)N1CC[C@@H](N2CC(C#Cc3c(-c4cc(OC)c(Cl)c(OC)c4)c4c(N)ncnc4n3C)C2)[C@@H](O)C1] | ¹H NMR (400 MHz, DMSO-d₆) δ 8.19 (s, 1H), 6.88 (s, 2H), 6.75 (ddd, J = 20.5, 16.7, 10.5 Hz, 1H), 6.36 (s, 2H), 6.05 (dd, J = 16.8, 2.4 Hz, 2H), 5.67 − 5.58 (m, 1H), 4.54 − 4.42 (m, 1H), 4.02 − 3.96 (m, 1H), 3.89 (s, 6H), 3.80 (dd, J = 13.7, 5.0 Hz, 1H), 3.73 (s, 3H), 3.72 − 3.59 (m, 1H), 3.59 (d, J = 6.0 Hz, 2H), 3.50 (t, J = 7.5 Hz, 1H), 3.31 − 3.11 (m, 1H), 3.09 (q, J = 7.7, 6.8 Hz, 2H), 2.89 (dt, J = 13.4, 6.8 Hz, 1H), 2.36 − 2.28 (m, 1H), 1.51- 1.38 (m, 2H). | 551.3 |
| 1-((3S,4R)-4-(3- ((4-amino-5-(6- methoxypyridin- 2-yl)-7-methyl- 7H-pyrrolo[2,3- d]pyrimidin-6- yl)ethynyl)azetidin- 1-yl)-3- hydroxypiperidin- 1-yl)prop-2- en-1-one | 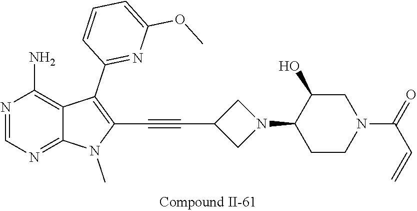 [SMILES: C=CC(=O)N1CC[C@@H](N2CC(C#Cc3c(-c4cccc(OC)n4)c4c(N)ncnc4n3C)C2)[C@@H](O)C1] | ¹H NMR (400 MHz, Methanol-d₄) δ 8.16 (s, 1H), 7.93 (d, J = 7.6 Hz, 1H), 7.81 (t, J = 7.9 Hz, 1H), 6.80 − 6.76 (m, 2H), 6.17 (dd, J = 16.8, 2.0 Hz, 1H), 5.73 − 5.70 (m, 1H), 4.50 − 4.36 (m, 1H), 4.11 − 4.03 (m, 1H), 4.00 (s, 3H), 3.83 (s, 5H), 3.77 (t, J = 7.1 Hz, 1H), 3.64 (p, J = 7.5 Hz, 1H), 3.37 (d, J = 6.2 Hz, 1H), 3.34 − 3.10 (m, 2H), 2.85 (dt, J = 14.0, 7.4 Hz, 1H), 2.51 − 2.42 (m, 1H), 1.60 (dt, J = 7.6, 4.6 Hz, 2H). | 488.4 |
| 1-((1R,5S,6s)-6- ((4-amino-5-(5- chloro-6- methoxypyridin- 2-yl)-7- isopropyl-7H- pyrrolo[2,3- d]pyrimidin-6- yl)ethynyl)-3- azabicyclo[3.1.0] hexan-3- yl)prop-2-en-1- one | 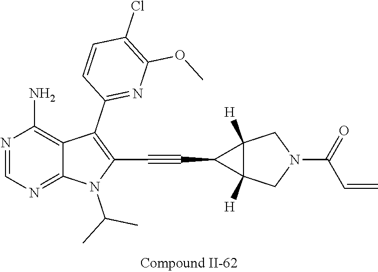 [SMILES: C=CC(=O)N1C[C@@H]2[C@@H](C#Cc3c(-c4ccc(Cl)c(OC)n4)c4c(N)ncnc4n3C(C)C)[C@@H]2C1] | ¹H NMR (400 MHz, DMSO-d₆): δ 8.12-8.15 (m, 1H), 7.98-8.08 (m, 1H), 7.62 (d, J =8.06 Hz, 1H), 7.35-7.48 (m, 2H), 6.40-6.62 (m, 2H), 5.94-6.21 (m, 3H), 5.57-5.70 (m, 2H), 5.00-5.19 (m, 2H), 3.97- 4.07 (m, 3H), 3.86-3.95 (m, 1H), 3.75-3.84 (m, 1H), 3.61-3.69 (m, 2H), 3.37-3.47 (m, 2H), 2.16-2.25 (m, 1H), 2.07- 2.15 (m, 1H), 1.54-1.67 (m, 6H), 1.19-1.26 (m, 1H). | 477 |
| 1-((3S,4R)-4-(3- ((4-amino-7- methyl-5-(1- methyl-1H- indazol-4-yl)- 7H-pyrrolo[2,3- d]pyrimidin-6- yl)ethynyl) azetidin-1-yl)-3- hydroxypiperidin- 1-yl)prop-2- en-1-one | 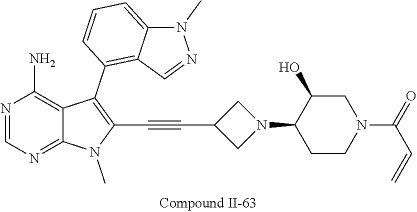 [SMILES: C=CC(=O)N1CC[C@@H](N2CC(C#Cc3c(-c4cccc5c4cnn5C)c4c(N)ncnc4n3C)C2)[C@@H](O)C1] | ¹H NMR (400 MHz, DMSO-d₆) δ 8.22 (s, 1H), 7.86 (d, J = 4.6 Hz, 1H), 7.70 (d, J = 8.4 Hz, 1H), 7.55 − 7.51 (m, 1H), 7.17 (d, J = 7.0 Hz, 1H), 6.72 (dd, J = 16.9, 10.5 Hz, 1H), 6.05 (d, J = 16.6 Hz, 1H), 5.62 (d, J = 10.3 Hz, 1H), 4.46 − 4.34 (s, 1H), 4.10 (s, 3H), 4.01 (d, J = 13.3 Hz, 1H), 3.77 (s, 3H), 3.54 (s, 3H), 3.18 (d, J = 13.4 Hz, 3H), 2.92 (s, 3H), 2.21 (s, 1H), 1.46 − 1.33 (s, 2H). | 511.4 |
| 1-((3S,4R)-4-(3- ((4-amino-5-(4- chloro-3- methylphenyl)- 7-methyl-7H- pyrrolo[2,3- d]pyrimidin-6- yl)ethynyl) azetidin-1-yl)-3- hydroxypiperidin- 1-yl)prop-2- en-1-one | 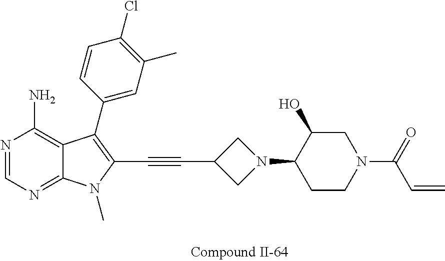 [SMILES: C=CC(=O)N1CC[C@@H](N2CC(C#Cc3c(-c4ccc(Cl)c(C)c4)c4c(N)ncnc4n3C)C2)[C@@H](O)C1] | ¹H NMR (400 MHz, DMSO-d₆) δ 8.19 (s, 1H), 7.57 − 7.48 (m, 2H), 7.37 − 7.30 (m, 1H), 6.83 − 6.67 (m, 1H), 6.21 (s, 1H), 6.05 (d, J = 17.0 Hz, 1H), 5.63 (d, J = 10.1 Hz, 1H), 4.53 − 4.41 (s, 1H), 4.00 − 3.80 (d, J = 14.7 Hz, 1H), 3.72 (s, 3H), 3.67 (d, J = 6.2 Hz, 1H), 3.58 (d, J = 7.5 Hz, 3H), 3.44 (t, J = 7.6 Hz, 1H), 3.21 (d, J = 13.5 Hz, 1H), 3.08 (q, J = 7.5, 6.7 Hz, 2H), 2.91 (s, 1H), 2.41 (s, 3H), 2.29 (s, 1H), 1.52 − 1.39 (s, 2H). | 505.4 |
| 1-((3S,4R)-4-(3- ((4-amino-5-(4- chloro-3- hydroxyphenyl)- 7-methyl-7H- pyrrolo[2,3- d]pyrimidin-6- yl)ethynyl) azetidin-1-yl)-3- hydroxypiperidin- 1-yl)prop-2- en-1-one | 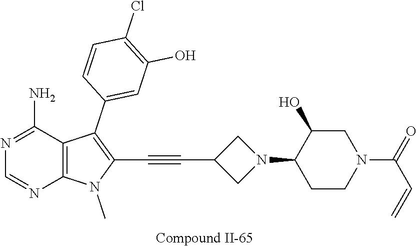 [SMILES: C=CC(=O)N1CC[C@@H](N2CC(C#Cc3c(-c4ccc(Cl)c(O)c4)c4c(N)ncnc4n3C)C2)[C@@H](O)C1] | ¹H NMR (400 MHz, DMSO-d₆) δ 10.39 (s, 1H), 8.18 (s, 1H), 7.44 (d, J = 8.2 Hz, 1H), 7.11 (d, J = 2.0 Hz, 1H), 6.92 (dd, J = 8.1, 2.0 Hz, 1H), 6.75 (ddd, J = 20.9, 16.7, 10.5 Hz, 1H), 6.25 (s, 2H), 6.05 (dd, J = 16.7, 2.5 Hz, 1H), 5.67 − 5.58 (m, 1H), 4.51 (s, 1H), 4.05 (d, J = 13.1 Hz, 1H), 3.82 (dd, J = 13.5, 4.8 Hz, 1H), 3.71 (s, 3H), 3.66 (s, 1H), 3.59 (t, J = 6.7 Hz, 2H), 3.45 (d, J = 7.7 Hz, 1H), 3.20 (d, J = 13.1 Hz, 1H), 3.10 (q, J = 7.3 Hz, 2H), 2.88 (dt, J = 13.7, 6.9 Hz, 1H), 2.38 − 2.30 (m, 1H), 1.52 − 1.38 (m, 2H). | 507.3 |
| 1-((3S,4R)-4-(3- ((4-amino-5-(4- chloro-3- fluorophenyl)-7- methyl-7H- pyrrolo[2,3- d]pyrimidin-6- yl)ethynyl) azetidin-1-yl)-3- hydroxypiperidin- 1-yl)prop-2- en-1-one | 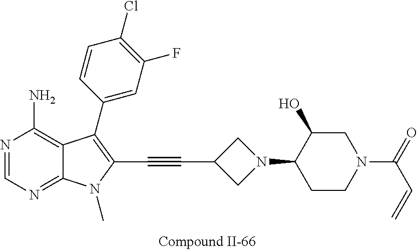 [SMILES: C=CC(=O)N1CC[C@@H](N2CC(C#Cc3c(-c4ccc(Cl)c(F)c4)c4c(N)ncnc4n3C)C2)[C@@H](O)C1] | ¹H NMR (400 MHz, DMSO-d₆) δ 8.19 (s, 1H), 7.68 (dd, J = 7.2, 2.2 Hz, 1H), 7.53 (t, J = 8.9 Hz, 1H), 7.46 (ddd, J = 8.5, 4.8, 2.2 Hz, 1H), 6.72 (dd, J = 16.6, 10.4 Hz, 1H), 6.33 (s, 2H), 6.05 (dd, J = 16.8, 2.5 Hz, 1H), 5.66 − 5.58 (m, 1H), 4.46 (dd, J = 49.0, 3.4 Hz, 1H), 4.02 (d, J = 13.0 Hz, 1H), 3.81 (dd, J = 13.7, 5.0 Hz, 1H), 3.72 (s, 4H), 3.60 (d, J = 6.7 Hz, 2H), 3.45 (t, J = 7.6 Hz, 1H), 3.24 − 3.04 (m, 3H), 2.93 − 2.85 (m, 1H), 2.34 (d, J = 32.6 Hz, 1H), 1.52 (s, 1H), 1.38 (s, 1H). | 509.2 |
| 1-((3S,4R)-4-(3- ((4-amino-5-(4- cyclopropyl-3- methoxyphenyl)- 7-methyl-7H- pyrrolo[2,3- d]pyrimidin-6- yl)ethynyl) azetidin-1-yl)-3- hydroxypiperidin- 1-yl)prop-2- en-1-one | 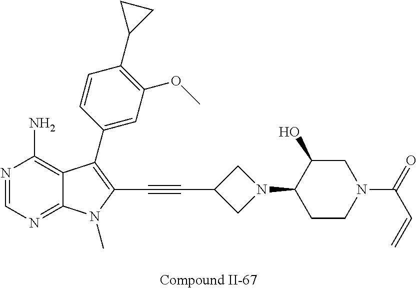 [SMILES: C=CC(=O)N1CC[C@@H](N2CC(C#Cc3c(-c4ccc(C5CC5)c(OC)c4)c4c(N)ncnc4n3C)C2)[C@@H](O)C1] | ¹H NMR (400 MHz, Methanol-d₄) δ 8.18 (s, 1H), 7.09 (s, 1H), 7.03 (d, J = 7.9 Hz, 1H), 6.97 (d, J = 7.8 Hz, 1H), 6.77 (dt, J = 16.8, 11.5 Hz, 1H), 6.17 (d, J = 16.5 Hz, 1H), 5.72 (t, J = 9.6 Hz, 1H), 4.51 − 4.36 (m, 1H), 4.12 − 3.95 (m, 1H), 3.92 (s, 3H), 3.86 − 3.73 (m, 5H), 3.69 (t, J = 7.2 Hz, 1H), 3.52 (p, J = 7.7 Hz, 1H), 3.27 − 3.14 (m, 3H), 3.09 − 2.76 (m, 1H), 2.40 (q, J = 8.3, 7.4 Hz, 1H), 2.23 (td, J = 8.5, 4.3 Hz, 1H), 1.54 (td, J = 8.9, 7.8, 4.2 Hz, 2H), 1.00 − 0.95 (m, 2H), 0.73 − 0.69 (m, 2H). | 527.25 |
| 1-((3S,4R)-4-(3- ((4-amino-5-(4- cyclopropoxy-3- methoxyphenyl)- 7-methyl-7H- pyrrolo[2,3- d]pyrimidin-6- yl)ethynyl)azetidin- 1-yl)-3- hydroxypiperidin- 1-yl)prop-2- en-1-one | 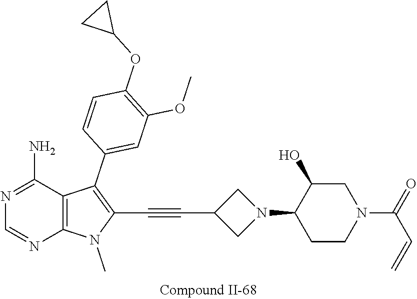 [SMILES: C=CC(=O)N1CC[C@@H](N2CC(C#Cc3c(-c4ccc(OC5CC5)c(OC)c4)c4c(N)ncnc4n3C)C2)[C@@H](O)C1] | ¹H NMR (400 MHz, DMSO-d₆) δ 8.18 (s, 1H), 7.37 (d, J = 8.2 Hz, 1H), 7.11 − 7.02 (m, 2H), 6.75 (ddd, J = 21.6, 16.6, 10.5 Hz, 1H), 6.20 − 6.05 (m, 1H), 5.63 (dt,J = 10.7, 3.4 Hz, 1H), 4.55 − 4.42 (m, 1H), 4.02 − 3.82 (m, 2H), 3.79 − 3.58 (m,10H), 3.46 (t, J = 7.6 Hz, 1H), 3.34 − 3.09 (m, 3H), 2.93 − 2.85 (m, 1H), 2.37 − 2.29 (s, 1H), 1.52 − 1.38 (m, 2H), 0.81 − 0.71 (m, 2H), 0.78 − 0.67 (m, 2H). | 543.40 |
| 1-((3S,4R)-4-(3- ((4-amino-5-(4- cyclopropylphenyl)- 7-methyl- 7H-pyrrolo[2,3- d]pyrimidin-6- yl)ethynyl) azetidin-1-yl)-3- hydroxypiperidin- 1-yl)prop-2- en-1-one | 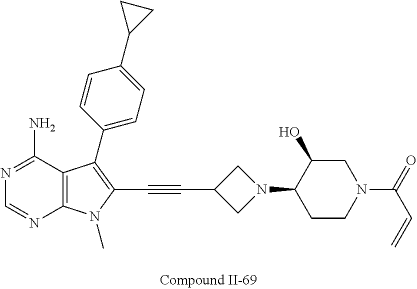 [SMILES: C=CC(=O)N1CC[C@@H](N2CC(C#Cc3c(-c4ccc(C5CC5)cc4)c4c(N)ncnc4n3C)C2)[C@@H](O)C1] | ¹H NMR (400 MHz, DMSO-d₆) δ 8.18 (s, 1H), 7.39 (d, J = 8.0 Hz, 2H), 7.22 (d, J = 8.0 Hz, 2H), 6.84 − 6.67 (m, 1H), 6.15 (d, J = 16.6 Hz, 2H), 6.05 (d, J = 16.6 Hz, 1H), 5.63 (d, J = 10.3 Hz, 1H), 4.52 (d, 1H), 3.95 (d, J = 12.5 Hz, 1H), 3.80 (s, J = 14.2 Hz, 3H), 3.58 (t, J = 7.2 Hz, 5H), 3.19 (m, 3H), 2.91 (m, 1H), 2.37 (m, 1H), 2.05 (m, 1H), 1.52 (m, 2H), 1.39 (d, J = 4.8 Hz, 5H), 1.05 − 0.96 (m, 2H), 0.79 − 0.70 (m, 2H). | 497.3 |
| 1-((3S,4R)-4-(3- ((4-amino-5-(4- cyclo- propoxyphenyl)- 7-methyl- 7H-pyrrolo[2,3- d]pyrimidin-6- yl)ethynyl)azetidin- 1-yl)-3- hydroxypiperidin- 1-yl)prop-2- en-1-one | 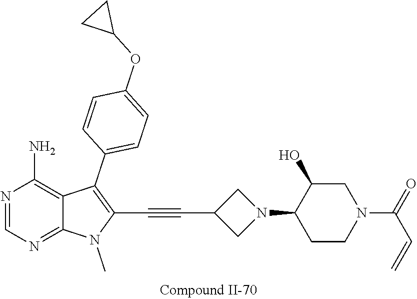 [SMILES: C=CC(=O)N1CC[C@@H](N2CC(C#Cc3c(-c4ccc(OC5CC5)cc4)c4c(N)ncnc4n3C)C2)[C@@H](O)C1] | ¹H NMR (400 MHz, Methanol-d₄) δ 8.18 (d, J = 1.1 Hz, 1H), 7.48 (d, J = 8.2 Hz, 2H), 7.21 (d, J = 8.2 Hz, 2H), 6.77 (dt, J = 16.8, 11.9 Hz, 1H), 6.22 − 6.14 (m, 1H), 5.77 − 5.68 (m, 1H), 4.51 − 4.37 (m, 1H), 4.03 (dd, J = 25.6, 13.8 Hz, 1H), 3.87 (dq, J = 6.1, 3.1 Hz, 1H), 3.80 (s, 4H), 3.74 (t, J = 7.2 Hz, 1H), 3.68 (t, J = 7.1 Hz, 1H), 3.50 (p, J = 7.7 Hz, 1H), 3.23 (dd, J = 12.4, 8.5 Hz, 3H), 3.08 − 2.76 (m, 1H), 2.46 − 2.37 (m, 1H), 1.71 − 1.52 (m, 2H), 0.85 (t, J = 6.0 Hz, 2H), 0.76 (q, J = 4.6 Hz, 2H). | 513.30 |
| 1-((3S,4R)-4-(3- ((4-amino-5-(4- isopropoxy-3- methoxyphenyl)- 7-methyl-7H- pyrrolo[2,3- d]pyrimidin-6- yl)ethynyl)azetidin- 1-yl)-3- hydroxypiperidin- 1-yl)prop-2- en-1-one | 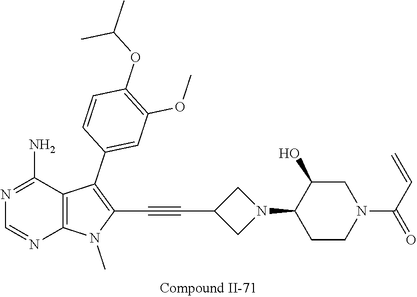 [SMILES: C=CC(=O)N1CC[C@@H](N2CC(C#Cc3c(-c4ccc(OC(C)C)c(OC)c4)c4c(N)ncnc4n3C)C2)[C@@H](O)C1] | ¹H NMR (400 MHz, DMSO-d₆) δ 8.18 (s, 1H), 7.09 (dd, J = 5.1, 3.1 Hz, 2H), 7.01 (dd, J = 8.2, 2.0 Hz, 1H), 6.73 (dd, J = 17.0, 10.3 Hz, 1H), 6.05 (d, J = 16.8 Hz, 1H), 5.63 (d, J = 10.4 Hz, 1H), 4.61 (p, J = 5.9 Hz, 1H), 4.41 (s, 1H), 4.05 (s, 1H), 3.80 (s, 3H), 3.72 (s, 3H), 3.60 (s, 4H), 3.22 (d, J = 13.9 Hz, 4H), 2.35 (s, 1H), 1.40 (s, 2H), 1.30 (d, J = 6.0 Hz, 6H). | 545.3 |
| 1-((3S,4R)-4-(3- ((4-amino-7- methyl-5-(4- (trifluoromethyl) phenyl)-7H- pyrrolo[2,3- d]pyrimidin-6- yl)ethynyl) azetidin-1-yl)-3- hydroxypiperidin- 1-yl)prop-2- en-1-one | 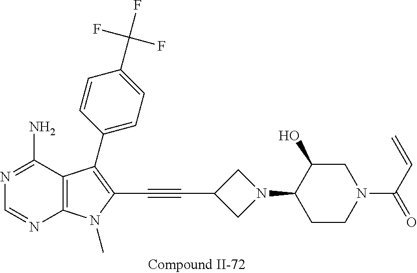 [SMILES: C=CC(=O)N1CC[C@@H](N2CC(C#Cc3c(-c4ccc(C(F)(F)F)cc4)c4c(N)ncnc4n3C)C2)[C@@H](O)C1] | ¹H NMR (400 MHz, DMSO-d₆) δ 8.22 (s, 1H), 7.86 (d, J = 8.0 Hz, 2H), 7.73 (d, J = 8.0 Hz, 2H), 6.83 − 6.67 (m, 1H), 6.29 (s, 2H), 6.05 (dd, J = 16.8, 2.5 Hz, 1H), 5.62 (dd, J = 10.4, 2.5 Hz, 1H), 4.47 (dd, J = 50.7, 3.4 Hz, 1H), 4.02 (d, J = 12.6 Hz, 1H), 3.81 (dd, J = 13.4, 5.0 Hz, 1H), 3.75 (s, 3H), 3.65 (s, 1H), 3.57 (d, J = 6.9 Hz, 2H), 3.45 (p, J = 7.6 Hz, 1H), 3.22 − 3.15 (m, 1H), 3.11 (dt, J = 13.8, 6.9 Hz, 2H), 2.93 − 2.85 (m, 1H), 2.33 (d, J = 31.8 Hz, 1H), 1.45 (d, J = 54.1 Hz, 2H). | 525.2 |
| 1-((3S,4R)-4-(3- ((4-amino-7- methyl-5-(4- (trifluoromethoxy) phenyl)-7H- pyrrolo[2,3- d]pyrimidin-6- yl)ethynyl) azetidin-1-yl)-3- hydroxypiperidin- 1-yl)prop-2- en-1-one | 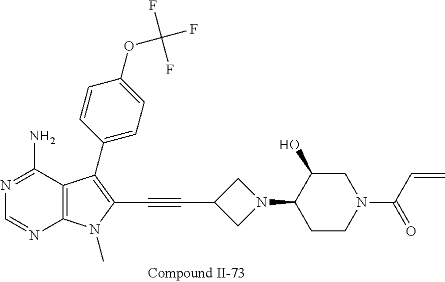 [SMILES: C=CC(=O)N1CC[C@@H](N2CC(C#Cc3c(-c4ccc(OC(F)(F)F)cc4)c4c(N)ncnc4n3C)C2)[C@@H](O)C1] | ¹H NMR (400 MHz, DMSO-d₆) δ 8.20 (s, 1H), 7.66 − 7.59 (m, 2H), 7.50 (d, J = 8.2 Hz, 2H), 6.83 − 6.67 (m, 1H), 6.21 (s, 1H), 6.05 (dd, J = 16.8, 2.4 Hz, 1H), 5.62 (dd, J = 10.4, 2.6 Hz, 1H), 4.46 (d, J = 50.8 Hz, 1H), 4.02 (d, J = 13.2 Hz, 1H), 3.80 (d, J = 14.2 Hz, 1H), 3.73 (s, 3H), 3.65 (s, 1H), 3.59 (d, J = 6.7 Hz, 2H), 3.45 (q, J = 7.5 Hz, 1H), 3.19 (d, J = 13.5 Hz, 1H), 3.08 (q, J = 8.8, 6.8 Hz, 2H), 2.88 (s, 1H), 2.28 (s, 1H), 1.44 (d, J = 52.7 Hz, 2H). | 541.3 |
| 1-((3S,4R)-4-(3- ((4-amino-5- (2,3- dihydrobenzo[b] [1,4]dioxin-6- yl)-7-methyl- 7H-pyrrolo[2,3- d]pyrimidin-6- yl)ethynyl) azetidin-1-yl)-3- hydroxypiperidin- 1-yl)prop-2- en-1-one | 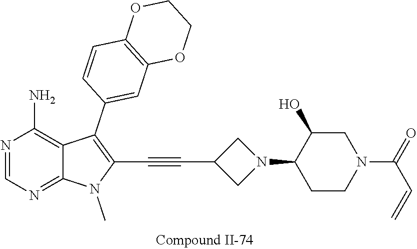 [SMILES: C=CC(=O)N1CC[C@@H](N2CC(C#Cc3c(-c4ccc5c(c4)OCCO5)c4c(N)ncnc4n3C)C2)[C@@H](O)C1] | ¹H NMR (400 MHz, DMSO-d₆) δ 8.17 (s, 1H), 7.03 − 6.97 (m, 2H), 6.75 (ddd, J = 20.1, 16.8, 10.6 Hz, 1H), 6.22 (s, 2H), 6.05 (dd, J = 16.7, 2.6 Hz, 1H), 5.63 (dt, J = 10.4, 3.4 Hz, 1H), 4.55 − 4.37 (m, 1H), 4.30 (s, 4H), 4.03 (d, J = 12.3 Hz, 1H), 3.81 (dd, J = 13.5, 4.9 Hz, 1H), 3.70 (s, 4H), 3.58 (d, J = 7.2 Hz, 2H), 3.44 (q, J = 7.6 Hz, 1H), 3.21 (d, J = 14.0 Hz, 1H), 3.10 (q, J = 6.7 Hz, 2H), 2.95 − 2.86 (m, 1H), 2.35 (d, J = 32.1 Hz, 1H), 1.56 − 1.34 (m, 2H). | 515.3 |
| 1-((3S,4R)-4-(3- ((4-amino-7- methyl-5-(4- methyl-3,4- dihydro-2H- benzo[b][1,4] oxazin-7-yl)-7H- pyrrolo[2,3- d]pyrimidin-6- yl)ethynyl)azetidin- 1-yl)-3- hydroxypiperidin- 1-yl)prop-2- en-1-one | 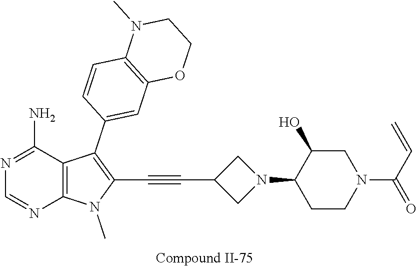 [SMILES: C=CC(=O)N1CC[C@@H](N2CC(C#Cc3c(-c4ccc5c(c4)OCCN5C)c4c(N)ncnc4n3C)C2)[C@@H](O)C1] | ¹H NMR (400 MHz, DMSO-d₆) δ 8.16 (s, 1H), 6.91 (d, J = 8.3 Hz, 1H), 6.88 (m, 1H), 6.79 (m, 1H), 6.73 (dd, J = 16.8, 10.4 Hz, 1H), 6.79 (s, 2H), 6.05 (d, J = 16.5 Hz, 1H), 5.63 (d, J = 10.1 Hz, 1H), 4.54 − 4.42 (d, 1H), 4.28 (s, 2H), 4.04 (m, 1H), 3.85 (d, J = 12.8 Hz, 1H), 3.69 (s, 4H), 3.60 (s, 2H), 3.49 (m, 1H), 3.31 (m, 2H), 3.20 (d, J = 13.8 Hz, 1H), 3.11 (s, 1H), 2.89 (s, 3H), 2.45 (m, 1H), 1.69 − 1.21 (m, 2H). | 528.2 |
| 1-((3S,4R)-4-(3- ((4-amino-5- (3,4- dimethoxyphenyl)- 7-methyl-7H- pyrrolo[2,3- d]pyrimidin-6- yl)ethynyl)azetidin- 1-yl)-3- hydroxypiperidin- 1-yl)prop-2- en-1-one | 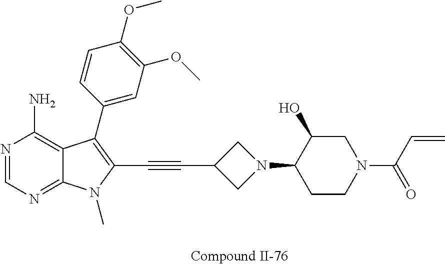 [SMILES: C=CC(=O)N1CC[C@@H](N2CC(C#Cc3c(-c4ccc(OC)c(OC)c4)c4c(N)ncnc4n3C)C2)[C@@H](O)C1] | ¹H NMR (400 MHz, DMSO-d₆) δ 8.18 (s, 1H), 7.10 (dd, J = 5.2, 3.1 Hz, 2H), 7.02 (dd, J = 8.2, 2.0 Hz, 1H), 6.75 (ddd, J = 20.1, 16.7, 10.5 Hz, 1H), 6.19 (s, 2H), 6.05 (dd, J = 16.7, 2.4 Hz, 1H), 5.62 (dt, J = 10.5, 2.6 Hz, 1H), 4.46 (dd, J = 51.1, 3.4 Hz, 1H), 4.02 (d, J = 13.3 Hz, 1H), 3.81 (d, J = 4.5 Hz, 7H), 3.72 (s, 3H), 3.66 (q, J = 7.7, 7.0 Hz, 1H), 3.65 − 3.40 (m, 3H), 3.24 − 3.02 (m, 3H), 2.89 (dt, J = 13.5, 6.8 Hz, 1H), 2.29 (s, 1H), 1.57 − 1.29 (m, 2H). | 517.3 |
| 1-((3S,4R)-4-(3- ((4-amino-7- methyl-5-(4- methyl-3,4- dihydro-2H- benzo[b][1,4] oxazin-6-yl)-7H- pyrrolo[2,3- d]pyrimidin-6- yl)ethynyl) azetidin-1-yl)-3- hydroxypiperidin- 1-yl)prop-2- en-1-one | 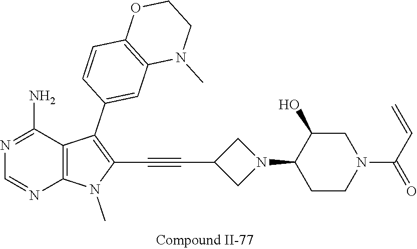 [SMILES: C=CC(=O)N1CC[C@@H](N2CC(C#Cc3c(-c4ccc5c(c4)N(C)CCO5)c4c(N)ncnc4n3C)C2)[C@@H](O)C1] | ¹H NMR (400 MHz, DMSO-d₆) δ 8.16 (s, 1H), 6.83 − 6.68 (m, 4H), 6.19 (s, 2H), 6.05 (dd, J = 16.9, 2.5 Hz, 1H), 5.66 − 5.58 (m, 1H), 4.45 (d, 1H), 4.29 (t, J = 4.3 Hz, 2H), 3.85 (d, J = 13.3 Hz, 1H), 3.70 (s, 3H), 3.55 (m, 3H), 3.45 (m, 1H), 3.35 (m, 1H), 3.31 (m, J = 9.0 Hz, 2H), 3.20 (d, J = 13.4 Hz, 1H), 3.10 (d, J = 7.4 Hz, 2H), 2.91 (s, 1H), 2.88 (s, 3H), 2.30 (s, 1H), 1.38 (s, 2H). | 528.2 |
| 1-((1R,5S,6s)-6- ((4-amino-3-(3- methoxy-4-((6- methylpyridin- 2- yl)oxy)phenyl)- 1-(2- methoxyethyl)- 1H-pyrrolo[3,2- c]pyridin-2- yl)ethynyl)-3- azabicyclo[3.1.0] hexan-3- yl)prop-2-en-1- one | 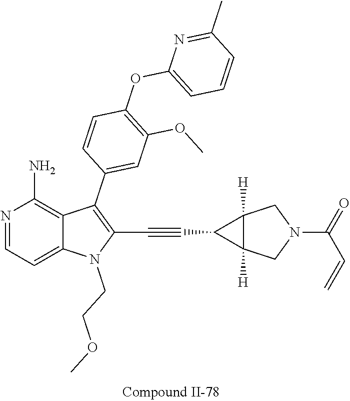 [SMILES: C=CC(=O)N1C[C@@H]2[C@@H](C#Cc3c(-c4ccc(Oc5cccc(C)n5)c(OC)c4)c4c(N)nccc4n3CCOC)[C@@H]2C1] | ¹H NMR (400 MHz, DMSO-d₆) δ 7.76 − 7.62 (m, 2H), 7.31 − 7.17 (m, 2H), 7.07 (d, J = 8.1 Hz, 1H), 6.96 (d, J = 7.3 Hz, 1H), 6.84 (d, J = 6.1 Hz, 1H), 6.74 (d, J = 8.1 Hz, 1H), 6.50 (dd, J = 16.8, 10.3 Hz, 1H), 6.10 (dd, J = 16.9, 2.4 Hz, 1H), 5.66 (d, J = 12.0 Hz, 1H), 4.35 (s, 2H), 4.35 (t, J = 5.6 Hz, 2H), 3.88 (d, J = 10.7 Hz, 1H), 3.76 (d, J = 3.5 Hz, 3H), 3.73 (s, 1H), 3.65 (dt, J = 18.0, 4.8 Hz, 3H), 3.40 (s, 1H), 3.25 (s, 3H), 2.33 (s, 3H), 2.08 (dt, J = 7.6, 3.8 Hz, 1H), 2.00 (dt, J = 7.2, 3.7 Hz, 1H), 1.50 (t, J = 3.5 Hz, 1H). | 564.40 |
| 1-((3R,4S)-4-(3- ((4-amino-3-(3- methoxy-4- phenoxyphenyl)- 1-methyl-1H- pyrrolo[3,2- c]pyridin-2- yl)ethynyl)azetidin- 1-yl)-3- hydroxypiperidin- 1-yl)prop-2- en-1-one | 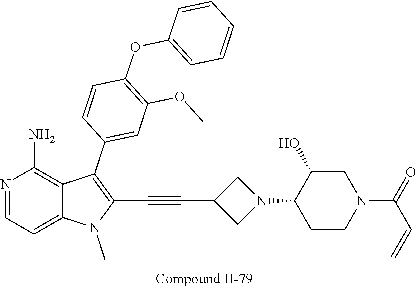 [SMILES: C=CC(=O)N1CC[C@H](N2CC(C#Cc3c(-c4ccc(Oc5ccccc5)c(OC)c4)c4c(N)nccc4n3C)C2)[C@H](O)C1] | ¹H NMR (400 MHz, DMSO-d₆) δ 7.72 (d, J = 5.9 Hz, 1H), 7.39 − 7.32 (m, 2H), 7.26 (d, J = 1.9 Hz, 1H), 7.15 (d, J = 8.3 Hz, 1H), 7.12 − 7.04 (m, 2H), 6.98 − 6.91 (m, 2H), 6.79 (d, J = 6.0 Hz, 1H), 6.33 (s, 1H), 6.04 (dd, J = 16.8, 2.4 Hz, 1H), 5.69 − 5.59 (m, 1H), 5.44 (s, 2H), 4.47 (dd, J = 50.3, 3.4 Hz, 1H), 4.00 (d, J = 13.6 Hz, 1H), 3.80 (s, 3H), 3.74 (s, 3H), 3.66 (d, J = 6.9 Hz, 1H), 3.63 − 3.54 (m, 2H), 3.46 (d, J = 7.6 Hz, 2H), 3.15 (d, J = 13.6 Hz, 1H), 3.12 − 3.00 (m, 2H), 2.83 (s, 1H), 2.25 (s, 1H), 1.55 − 1.23 (m, 2). | 578.40 |
| 1-(4-(3-((4- amino-3-(3- methoxy-4-(6- methylpyridin-2- yloxy)phenyl)- 1-methyl-1H- pyrrolo[3,2- c]pyridin-2- yl)ethynyl) azetidin-1- yl)piperidin-1- yl)prop-2-en-1- one | 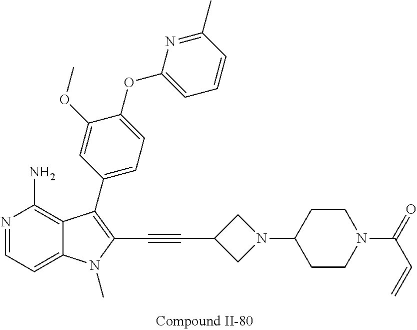 [SMILES: C=CC(=O)N1CCC(N2CC(C#Cc3c(-c4ccc(Oc5cccc(C)n5)c(OC)c4)c4c(N)nccc4n3C)C2)CC1] | ¹H NMR (400 MHz, Chloroform-d) δ 7.85 (d, J = 6.1 Hz, 1H), 7.58 (dd, J = 8.2, 7.4 Hz, 1H), 7.26 − 7.21 (m, 2H), 7.17 (dd, J = 8.0, 2.0 Hz, 1H), 6.90 (d, J = 7.4 Hz, 1H), 6.70 (d, J = 6.1 Hz, 1H), 6.65 (d, J = 8.2 Hz, 1H), 6.58 (dd, J = 16.8, 10.6 Hz, 1H), 6.27 (dd, J = 16.8, 1.9 Hz, 1H), 5.69 (dd, J = 10.5, 2.0 Hz, 1H), 5.01 (s, 2H), 4.26 (d, J = 12.9 Hz, 1H), 3.82 (d, J = 17.6 Hz, 7H), 3.67 (t, J = 7.0 Hz, 2H), 3.47 (p, J = 7.8 Hz, 1H), 3.18 (t, J = 11.8 Hz, 1H), 3.07 (t, J = 7.5 Hz, 3H), 2.47 (s, 3H), 2.30 (tt, J = 8.7, 3.8 Hz, 1H), 1.68 (s, 2H), 1.33 − 1.18 (m, 2H). | 577.3 |
| 1-(4-(3-((4- amino-5-(3- methoxy-4-((6- methylpyridin-2- yl)oxy)phenyl)- 7-methyl-7H- pyrrolo[2,3- d]pyrimidin-6- yl)ethynyl) azetidin-1- yl)piperidin-1- yl)prop-2-en-1- one | 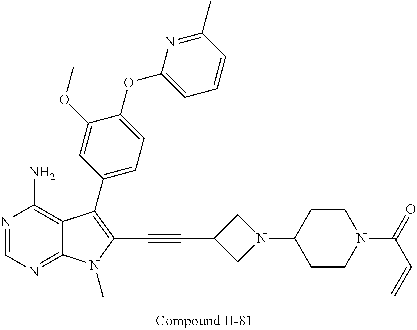 [SMILES: C=CC(=O)N1CCC(N2CC(C#Cc3c(-c4ccc(Oc5cccc(C)n5)c(OC)c4)c4c(N)ncnc4n3C)C2)CC1] | ¹H NMR (400 MHz, DMSO-d₆) δ 8.20 (s, 1H), 7.68 (t, J = 7.8 Hz, 1H), 7.29 − 7.21 (m, 2H), 7.12 (d, J = 8.2 Hz, 1H), 6.95 (d, J = 7.3 Hz, 1H), 6.83 − 6.69 (m, 2H), 6.30 (s, 1H), 6.06 (d, J = 16.7 Hz, 1H), 5.64 (d, J = 10.8 Hz, 1H), 3.97 (d, J = 13.1 Hz, 1H), 3.76 (s, 3H), 3.74 (s, 3H), 3.57 (t, J = 6.7 Hz, 2H), 3.48 (q, J = 7.3 Hz, 1H), 3.13 (t, J = 11.6 Hz, 1H), 3.02 (t, J = 6.7 Hz, 2H), 2.94 (t, J = 11.1 Hz, 1H), 2.32 (s, 3H), 2.24 (s, 1H), 1.61 − 1.55 (m, 2H), 1.03 (d, J = 15.9 Hz, 2H). | 578.4 |
| (3S,4R)-4-(3- ((4-amino-5-(3- methoxy-4- phenoxyphenyl)- 7-methyl-7H- pyrrolo[2,3- d]pyrimidin-6- yl)ethynyl)azetidin- 1-yl)-1- (vinylsulfonyl) piperidin-3-ol | 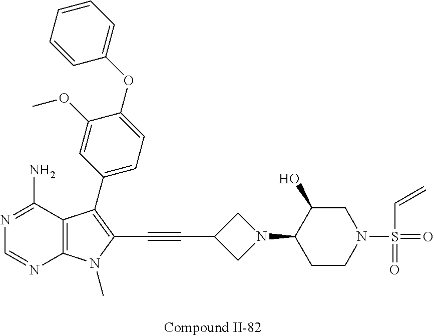 [SMILES: C=CS(=O)(=O)N1CC[C@@H](N2CC(C#Cc3c(-c4ccc(Oc5ccccc5)c(OC)c4)c4c(N)ncnc4n3C)C2)[C@@H](O)C1] | ¹H NMR (400 MHz, DMSO-d₆) δ 8.20 (s, 1H), 7.34 (t, J = 7.7 Hz, 2H), 7.27 (s, 1H), 7.10 (dt, J = 26.8, 7.8 Hz, 3H), 6.97 (d, J = 8.0 Hz, 2H), 6.80 (dd, J = 16.5, 10.1 Hz, 1H), 6.33 (s, 1H), 6.20 − 6.04 (m, 2H), 4.77 (s, 1H), 3.80 (s, 3H), 3.73 (s, 3H), 3.69 − 3.53 (m, 3H), 3.47 (t, J = 7.6 Hz, 1H), 3.20 − 3.08 (m, 2H), 3.06 − 2.92 (m, 3H), 2.87 (s, 1H), 2.29 (s, 1H), 1.50 (d, J = 48.2 Hz, 2H). | 615.35 |
| 5-(3-methoxy-4- phenoxyphenyl- 7-methyl-6-((1- (1- (vinylsulfonyl) piperidin-4- yl)azetidin-3- yl)ethynyl)-7H- pyrrolo[2,3- d]pyrimidin-4- amine | 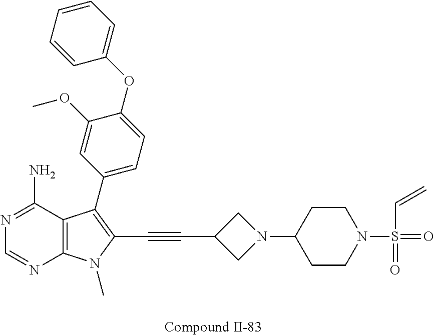 [SMILES: C=CS(=O)(=O)N1CCC(N2CC(C#Cc3c(-c4ccc(Oc5ccccc5)c(OC)c4)c4c(N)ncnc4n3C)C2)CC1] | ¹H NMR (400 MHz, DMSO-d₆) δ 8.20 (s, 1H), 7.34 (t, J = 7.8 Hz, 2H), 7.27 (s, 1H), 7.19 − 7.02 (m, 3H), 6.96 (d, J = 8.0 Hz, 2H), 6.77 (dd, J = 16.5, 10.1 Hz, 1H), 6.33 (s, 1H), 6.21 − 6.03 (m, 2H), 3.80 (s, 3H), 3.73 (s, 3H), 3.55 (t, J = 6.9 Hz, 2H), 3.47 (q, J = 7.4 Hz, 1H), 3.30 − 3.19 (m, 2H), 2.99 (d, J = 6.9 Hz, 2H), 2.75 (t, J = 10.5 Hz, 2H), 2.10 (d, J = 19.5 Hz, 1H), 1.62 (s, 2H), 1.22 (d, J = 10.7 Hz, 2H). | 599.35 |
| 1-(4-(3-((4- amino-5-(4- chlorophenyl)- 7-isopropyl-7H- pyrrolo[2,3- d]pyrimidin-6- yl)ethynyl) azetidin-1- yl)piperidin-1- yl)prop-2-en-1- one | 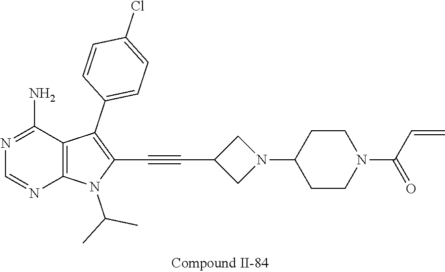 [SMILES: C=CC(=O)N1CCC(N2CC(C#Cc3c(-c4ccc(Cl)cc4)c4c(N)ncnc4n3C(C)C)C2)CC1] | (400 MHz, Chloroform-d) δ 8.34 (s, 1H), 7.55 − 7.44 (m, 4H), 6.59 (dd, J = 16.8, 10.6 Hz, 1H), 6.27 (dd, J = 16.9, 2.0 Hz, 1H), 5.69 (dd, J = 10.6, 1.9 Hz, 1H), 5.22 (hept, J = 7.0 Hz, 1H), 5.12 (s, 1H), 4.24 (s, 1H), 3.88 (d, J = 13.4 Hz, 1H), 3.69 (t, J = 7.0 Hz, 2H), 3.45 (p, J = 7.7 Hz, 1H), 3.23 (s, 1H), 3.08 (t, J = 7.1 Hz, 3H), 2.33 (dt, J = 8.9, 4.9 Hz, 1H), 1.72 (d, J = 6.9 Hz, 8H), 1.31 (dtd, J = 13.2, 9.3, 4.0 Hz, 2H). | 503.2 |
| 1-(4-(3-((4- amino-5-(4- chlorophenyl)- 7-(2- methoxyethyl)- 7H-pyrrolo[2,3- d]pyrimidin-6- yl)ethynyl) azetidin-1- yl)piperidin-1- yl)prop-2-en-1- one | 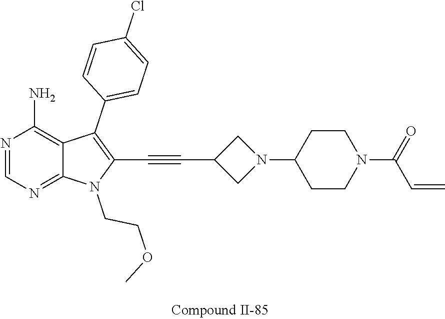 [SMILES: C=CC(=O)N1CCC(N2CC(C#Cc3c(-c4ccc(Cl)cc4)c4c(N)ncnc4n3CCOC)C2)CC1] | ¹H NMR (400 MHz, DMSO-d₆) δ 8.19 (s, 1H), 7.58 − 7.51 (m, 4H), 6.80 (dd, J = 16.7, 10.5 Hz, 1H), 6.25 (br, 1H), 6.08 (dd, J = 16.7, 2.5 Hz, 1H), 5.66 (dd, J = 10.4, 2.4 Hz, 1H), 4.39 (t, J = 5.9 Hz, 2H), 3.98 (d, J = 12.8 Hz, 1H), 3.82 (d, J = 13.4 Hz, 1H), 3.74 (t, J = 5.8 Hz, 2H), 3.55 (t, J = 6.9 Hz, 2H), 3.42 (p, J = 7.3 Hz, 1H), 3.27 (s, 3H), 3.20 (t, J = 11.5 Hz, 1H), 3.02 (q, J = 8.5, 6.8 Hz, 3H), 2.27 (dt, J = 8.7, 4.7 Hz, 1H), 1.62 (s, 2H), 1.08 (s, 2H). | 519.30 |
| 1-(4-(3-((4- amino-5-(4- chloro-3- methoxyphenyl)- 7-isopropyl- 7H-pyrrolo[2,3- d]pyrimidin-6- yl)ethynyl) azetidin-1- yl)piperidin-1- yl)prop-2-en-1- one | 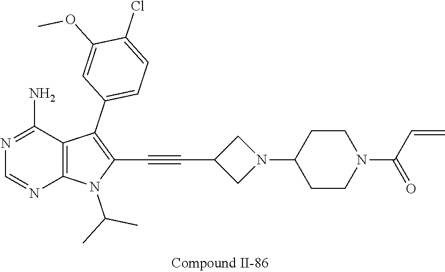 [SMILES: C=CC(=O)N1CCC(N2CC(C#Cc3c(-c4ccc(Cl)c(OC)c4)c4c(N)ncnc4n3C(C)C)C2)CC1] | (400 MHz, Chloroform-d) δ 8.35 (s, 1H), 7.48 (d, J = 8.0 Hz, 1H), 7.15 (d, J = 1.9 Hz, 1H), 7.11 (dd, J = 8.1, 1.9 Hz, 1H), 6.59 (dd, J = 16.8, 10.6 Hz, 1H), 6.27 (dd, J = 16.8, 1.9 Hz, 1H), 5.69 (dd, J = 10.5, 2.0 Hz, 1H), 5.23 (hept, J = 6.9 Hz, 1H), 5.11 (s, 2H), 4.25 (d, J = 13.3 Hz, 1H), 3.97 (s, 3H), 3.87 (d, J = 13.4 Hz, 1H), 3.66 (t, J = 7.0 Hz, 2H), 3.53 − 3.38 (m, 1H), 3.22 (t, J = 11.7 Hz, 1H), 3.07 (q, J = 9.6, 7.1 Hz, 3H), 2.30 (tt, J = 8.5, 3.8 Hz, 1H), 1.72 (d, J = 6.8 Hz, 8H), 1.29 (dtd, J = 13.2, 9.3, 3.9 Hz, 2H). | 533.2 |
| 1-(4-(3-((4- amino-5-(4- chloro-3- methoxyphenyl)- 7-(2- methoxyethyl)- 7H-pyrrolo[2,3- d]pyrimidin-6- yl)ethynyl) azetidin-1- yl)piperidin-1- yl)prop-2-en-1- one | 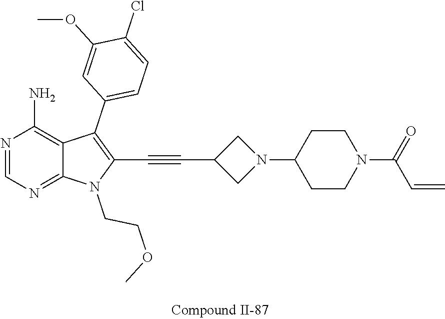 [SMILES: C=CC(=O)N1CCC(N2CC(C#Cc3c(-c4ccc(Cl)c(OC)c4)c4c(N)ncnc4n3CCOC)C2)CC1] | ¹H NMR (400 MHz, DMSO-d₆) δ 8.20 (s, 1H), 7.54 (d, J = 8.2 Hz, 1H), 7.23 (d, J = 1.9 Hz, 1H), 7.10 (dd, J = 8.1, 1.9 Hz, 1H), 6.80 (dd, J = 16.7, 10.5 Hz, 1H), 6.32 (br, 2H), 6.08 (dd, J = 16.7, 2.5 Hz, 1H), 5.65 (dd, J = 10.4, 2.5 Hz, 1H), 4.40 (t, J = 5.9 Hz, 2H), 3.96 (d, J = 13.0 Hz, 1H), 3.91 (s, 3H), 3.83 (d, J = 13.7 Hz, 1H), 3.75 (t, J = 5.9 Hz, 2H), 3.56 (t, J = 6.8 Hz, 2H), 3.45 (p, J = 7.3 Hz, 1H), 3.27 (s, 3H), 3.20 (t, J = 10.7 Hz, 1H), 3.01 (t, J = 6.7 Hz, 3H), 2.26 (dt, J = 8.6, 4.9 Hz, 1H), 1.61 (s, 2H), 1.07 (s, 2H) | 549.35 |
| 1-(4-(3-((4- amino-7- isopropyl-5-(4- (oxetan-3- yloxy)phenyl)- 7H-pyrrolo[2,3- d]pyrimidin-6- yl)ethynyl) azetidin-1- yl)piperidin-1- yl)prop-2-en-1- one | 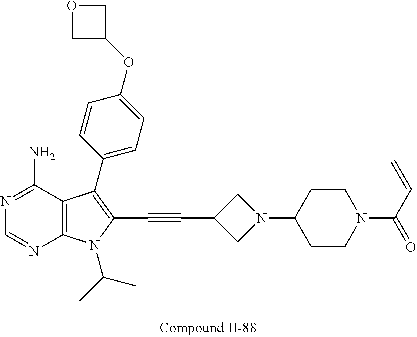 [SMILES: C=CC(=O)N1CCC(N2CC(C#Cc3c(-c4ccc(OC5COC5)cc4)c4c(N)ncnc4n3C(C)C)C2)CC1] | ¹H NMR (400 MHz, Methanol-d₄) δ 8.18 (s, 1H), 7.52 − 7.46 (m, 2H), 7.00 − 6.92 (m, 2H), 6.79 (dd, J = 16.8, 10.6 Hz, 1H), 6.21 (dd, J = 16.9, 1.9 Hz, 1H), 5.77 (dd, J = 10.6, 2.0 Hz, 1H), 5.37 (p, J = 5.4 Hz, 1H), 5.19 (p, J = 7.0 Hz, 1H), 5.08 (t, J = 6.6 Hz, 2H), 4.75 (dd, J = 7.2, 4.9 Hz, 2H), 4.55 (d, J = 13.5 Hz, 1H), 4.15 (q, J = 10.6, 8.0 Hz, 3H), 3.86 − 3.70 (m, 3H), 3.20 (t, J = 12.9 Hz, 1H), 3.00 (s, 1H), 2.84 (t, J = 12.6 Hz, 1H), 1.97 (d, J = 12.7 Hz, 2H), 1.71 (d, J = 6.9 Hz, 6H), 1.37 − 1.22 (m, 2H). | 541.3 |
| 1-(4-(3-((4- amino-5-(4- chloro-3- (fluoromethyl) phenyl)-7- methyl-7H- pyrrolo[2,3- d]pyrimidin-6- yl)ethynyl) azetidin-1- yl)piperidin-1- yl)prop-2-en-1- one | 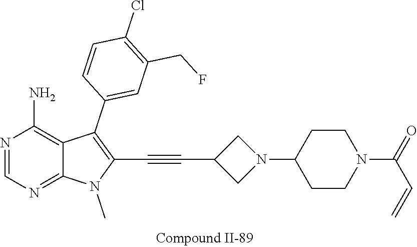 [SMILES: C=CC(=O)N1CCC(N2CC(C#Cc3c(-c4ccc(Cl)c(CF)c4)c4c(N)ncnc4n3C)C2)CC1] | ¹H NMR (400 MHz, DMSO-d₆) δ 8.20 (s, 1H), 7.72 (t, J = 1.8 Hz, 1H), 7.65 (dd, J = 8.2, 1.1 Hz, 1H), 7.53 (dt, J = 8.2, 1.8 Hz, 1H), 6.79 (dd, J = 16.7, 10.5 Hz, 1H), 6.29 (s, 1H), 6.07 (dd, J = 16.7, 2.5 Hz, 1H), 5.67 − 5.63 (m, 2H), 5.53 (s, 1H), 3.99 (d, J = 13.1 Hz, 1H), 3.83 (d, J = 13.9 Hz, 1H), 3.73 (s, 3H), 3.56 (t, J = 6.9 Hz, 2H), 3.43 (p, J = 7.5 Hz, 1H), 3.18 (s, 1H), 3.01 (q, J = 11.0, 8.8 Hz, 3H), 2.26 (dt, J = 8.8, 4.7 Hz, 1H), 1.60 (s, 2H), 1.08 (d, J = 11.6 Hz, 2H). | 507.3 |
| 1-(4-(3-((4- amino-5-(4- chloro-3- (hydroxymethyl) phenyl)-7- methyl-7H- pyrrolo[2,3- d]pyrimidin-6- yl)ethynyl) azetidin-1- yl)piperidin-1- yl)prop-2-en-1- one | 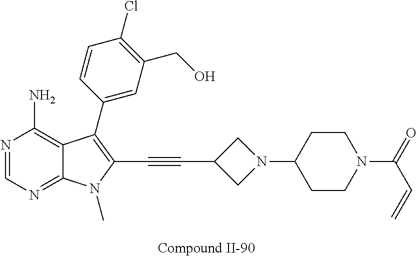 [SMILES: C=CC(=O)N1CCC(N2CC(C#Cc3c(-c4ccc(Cl)c(CO)c4)c4c(N)ncnc4n3C)C2)CC1] | ¹H NMR (400 MHz, DMSO-d₆) δ 8.20 (s, 1H), 7.74 (d, J = 2.2 Hz, 1H), 7.54 (d, J = 8.2 Hz, 1H), 7.41 (dd, J = 8.2, 2.3 Hz, 1H), 6.81 (dd, J = 16.7, 10.5 Hz, 1H), 6.24 (s, 1H), 6.08 (dd, J = 16.7, 2.5 Hz, 1H), 5.65 (dd, J = 10.4, 2.5 Hz, 1H), 5.49 (t, J = 5.5 Hz, 1H), 4.64 (d, J = 5.1 Hz, 2H), 4.02 − 3.96 (m, 1H), 3.84 (d, J = 13.5 Hz, 1H), 3.73 (s, 3H), 3.56 (t, J = 6.9 Hz, 2H), 3.43 (p, J = 7.5 Hz, 1H), 3.18 (d, J = 5.2 Hz, 1H), 3.08 − 2.94 (m, 3H), 2.28 (dt, J = 8.8, 4.8 Hz, 1H), 1.61 (s, 2H), 1.08 (s, 2H). | 505.3 |
| 1-(4-(3-((4- amino-5-(4- chloro-3,5- dimethoxyphenyl)- 7-methyl-7H- pyrrolo[2,3- d]pyrimidin-6- yl)ethynyl) azetidin-1- yl)piperidin-1- yl)prop-2-en-1- one | 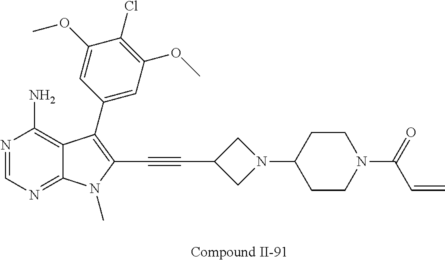 [SMILES: C=CC(=O)N1CCC(N2CC(C#Cc3c(-c4cc(OC)c(Cl)c(OC)c4)c4c(N)ncnc4n3C)C2)CC1] | ¹H NMR (400 MHz, DMSO-d₆) δ 8.20 (s, 1H), 6.88 (s, 2H), 6.79 (dd, J = 16.7, 10.5 Hz, 1H), 6.36 (s, 2H), 6.07 (dd, J = 16.7, 2.5 Hz, 1H), 5.65 (dd, J = 10.5, 2.4 Hz, 1H), 4.00 (d, J = 12.8 Hz, 1H), 3.89 (s, 6H), 3.83 (d, J = 13.4 Hz, 1H), 3.73 (s, 3H), 3.57 (t, J = 6.8 Hz, 2H), 3.48 (p, J = 7.3 Hz, 1H), 3.17 (t, J = 11.5 Hz, 1H), 3.06 − 2.93 (m, 3H), 2.26 (dt, J = 8.9, 4.9 Hz, 1H), 1.61 (s, 1H), 1.07 (s, 3H). | 535.2 |
| 1-(4-(3-((4- amino-5-(6- methoxypyridin- 2-yl)-7-methyl- 7H-pyrrolo[2,3- d]pyrimidin-6- yl)ethynyl) azetidin-1- yl)piperidin-1- yl)prop-2-en-1- one | 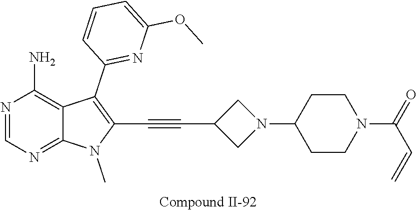 [SMILES: C=CC(=O)N1CCC(N2CC(C#Cc3c(-c4cccc(OC)n4)c4c(N)ncnc4n3C)C2)CC1] | ¹H NMR (400 MHz, DMSO-d₆) δ 8.61 (s, 1H), 8.16 (s, 1H), 7.87 (d, J = 4.5 Hz, 2H), 7.45 (s, 1H), 6.89 − 6.73 (m, 2H), 6.07 (dd, J = 16.8, 2.4 Hz, 1H), 5.65 (dd, J = 10.3, 2.5 Hz, 1H), 4.00 (d, J = 13.1 Hz, 1H), 3.91 (s, 3H), 3.84 (d, J = 13.9 Hz, 1H), 3.73 (s, 3H), 3.59 (dt, J = 26.1, 7.4 Hz, 3H), 3.20 (s, 1H), 3.13 (t, J = 6.3 Hz, 2H), 3.01 (t, J = 11.4 Hz, 1H), 2.32 (s, 1H), 1.64 (s, 2H), 1.10 (s, 2H). | 472.3 |
| 1-(4-(3-((4- amino-7- methyl-5-(1- methyl-1H- indazol-4-yl)- 7H-pyrrolo[2,3- d]pyrimidin-6- yl)ethynyl) azetidin-1- yl)piperidin-1- yl)prop-2-en-1- one | 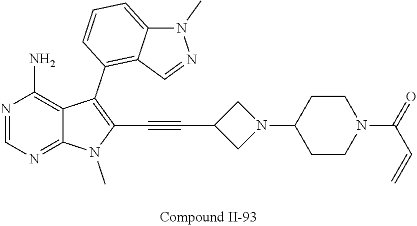 [SMILES: C=CC(=O)N1CCC(N2CC(C#Cc3c(-c4cccc5c4cnn5C)c4c(N)ncnc4n3C)C2)CC1] | ¹H NMR (400 MHz, Methanol-d₄) δ 8.26 (s, 1H), 7.97 (s, 1H), 7.69 (d, J = 8.6 Hz, 1H), 7.60 (dd, J = 8.5, 7.0 Hz, 1H), 7.28 (d, J = 6.8 Hz, 1H), 6.78 (dd, J = 16.7, 10.6 Hz, 1H), 6.21 (dd, J = 16.8, 1.9 Hz, 1H), 5.77 (dd, J = 10.6, 1.9 Hz, 1H), 4.56 (d, J = 13.9 Hz, 1H), 4.16 (s, 4H), 4.07 (dd, J = 18.2, 10.4 Hz, 2H), 3.91 (s, 3H), 3.72 (dt, J = 11.7, 6.7 Hz, 2H), 3.59 (s, 1H), 3.20 (d, J = 13.1 Hz, 1H), 3.01 (s, 1H), 2.82 (t, J = 12.5 Hz, 1H), 1.94 (s, 2H), 1.22 (s, 2H). | 495.3 |
| 1-(4-(3-((4- amino-5-(4- chloro-3- methylphenyl)- 7-methyl-7H- pyrrolo[2,3- d]pyrimidin-6- yl)ethynyl) azetidin-1- yl)piperidin-1- yl)prop-2-en-1- one | 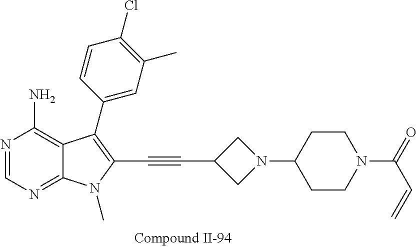 [SMILES: C=CC(=O)N1CCC(N2CC(C#Cc3c(-c4ccc(Cl)c(C)c4)c4c(N)ncnc4n3C)C2)CC1] | ¹H NMR (400 MHz, DMSO-d₆) δ 8.19 (s, 1H), 7.57 − 7.47 (m, 2H), 7.36 − 7.30 (m, 1H), 6.79 (dd, J = 16.7, 10.5 Hz, 1H), 6.21 (s, 1H), 6.07 (dd, J = 16.8, 2.5 Hz, 1H), 5.65 (dd, J = 10.3, 2.5 Hz, 1H), 3.99 (d, J = 13.1 Hz, 1H), 3.83 (d, J = 13.8 Hz, 1H), 3.72 (s, 3H), 3.56 (t, J = 6.8 Hz, 2H), 3.44 (d, J = 7.6 Hz, 1H), 3.17 (d, J = 11.2 Hz, 1H), 3.04 − 2.97 (m, 3H), 2.40 (s, 3H), 2.26 (s, 1H), 1.61 (s, 2H), 1.07 (s, 2H). | 489.3 |
| 1-(4-(3-((4- amino-5-(4- chloro-3- hydroxyphenyl)- 7-methyl-7H- pyrrolo[2,3- d]pyrimidin-6- yl)ethynyl)azeti din-1- yl)piperidin-1- yl)prop-2-en-1- one | 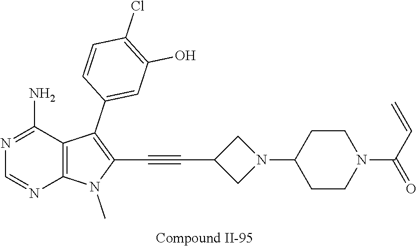 [SMILES: C=CC(=O)N1CCC(N2CC(C#Cc3c(-c4ccc(Cl)c(O)c4)c4c(N)ncnc4n3C)C2)CC1] | ¹H NMR (400 MHz, DMSO-d₆) δ 10.41 (s, 1H), 8.18 (s, 1H), 7.44 (d, J = 8.1 Hz, 1H), 7.12 (d, J = 1.8 Hz, 1H), 6.92 (dd, J = 8.2, 1.8 Hz, 1H), 6.79 (dd, J = 16.7, 10.5 Hz, 1H), 6.25 (s, 2H), 6.12 − 6.02 (m, 1H), 5.65 (dd, J = 10.1, 2.4 Hz, 1H), 3.99 (d, J = 12.8 Hz, 1H), 3.83 (d, J = 13.7 Hz, 1H), 3.71 (s, 3H), 3.57 (t, J = 6.9 Hz, 2H), 3.44 (q, J = 7.6 Hz, 1H), 3.18 (t, J = 11.7 Hz, 1H), 3.03 (d, J = 6.9 Hz, 3H), 2.28 (q, J = 5.3 Hz, 1H), 1.61 (s, 2H), 1.07 (s, 2H). | 419.3 |
| 1-(4-(3-((4- amino-5-(4- chloro-3- fluorophenyl)-7- methyl-7H- pyrrolo[2,3- d]pyrimidin-6- yl)ethynyl) azetidin-1- yl)piperidin-1- yl)prop-2-en-1- one | 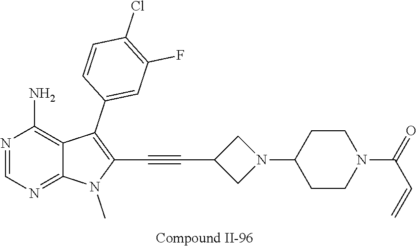 [SMILES: C=CC(=O)N1CCC(N2CC(C#Cc3c(-c4ccc(Cl)c(F)c4)c4c(N)ncnc4n3C)C2)CC1] | ¹H NMR (400 MHz, DMSO-d₆) δ 8.19 (s, 1H), 7.68 (d, J = 7.0 Hz, 1H), 7.54 (t, J = 9.0 Hz, 1H), 7.45 (s, 1H), 6.79 (dd, J = 16.7, 10.5 Hz, 1H), 6.35 (s, 2H), 6.07 (dd, J = 16.6, 2.5 Hz, 1H), 5.65 (dd, J = 10.4, 2.4 Hz, 1H), 3.98 (d, J = 12.9 Hz, 1H), 3.82 (d, J = 13.6 Hz, 1H), 3.71 (s, 3H), 3.57 (t, J = 7.1 Hz, 2H), 3.48 − 3.40 (m, 1H), 3.18 (t, J = 11.6 Hz, 1H), 3.01 (t, J = 6.8 Hz, 3H), 2.26 (d, J = 10.0 Hz, 1H), 1.61 (s, 2H), 1.08 (s, 2H). | 493.2 |
| 1-(4-(3-((4- amino-5-(4- cyclopropyl-3- methoxyphenyl)- 7-methyl-7H- pyrrolo[2,3- d]pyrimidin-6- yl)ethynyl) azetidin-1- yl)piperidin-1- yl)prop-2-en-1- one | 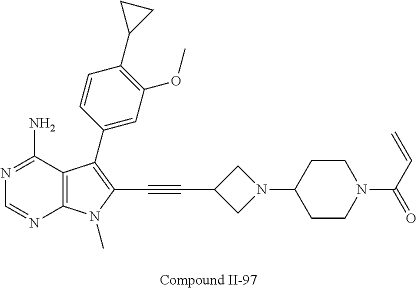 [SMILES: C=CC(=O)N1CCC(N2CC(C#Cc3c(-c4ccc(C5CC5)c(OC)c4)c4c(N)ncnc4n3C)C2)CC1] | ¹H NMR (400 MHz, DMSO-d₆) δ 8.18 (s, 1H), 7.07 (d, J = 1.6 Hz, 1H), 7.03 − 6.91 (m, 2H), 6.79 (dd, J = 16.7, 10.5 Hz, 1H), 6.07 (dd, J = 16.7, 2.5 Hz, 1H), 5.65 (dd, J = 10.4, 2.5 Hz, 1H), 3.99 (d, J = 12.8 Hz, 1H), 3.86 (s, 4H), 3.72 (s, 3H), 3.56 (t, J = 6.9 Hz, 2H), 3.44 (p, J = 7.2 Hz, 1H), 3.18 (t, J = 11.4 Hz, 1H), 3.01 (t, J = 6.8 Hz, 3H), 2.26 (s, 1H), 2.16 (td, J = 8.6, 4.5 Hz, 1H), 1.61 (s, 1H), 1.07 (s, 2H), 0.98 − 0.89 (m, 2H), 0.73 − 0.65 (m, 2H). | 511.4 |
| 1-(4-(3-((4- amino-5-(4- cyclopropoxy-3- methoxyphenyl)- 7-methyl-7H- pyrrolo[2,3- d]pyrimidin-6- yl)ethynyl) azetidin-1- yl)piperidin-1- yl)prop-2-en-1- one | 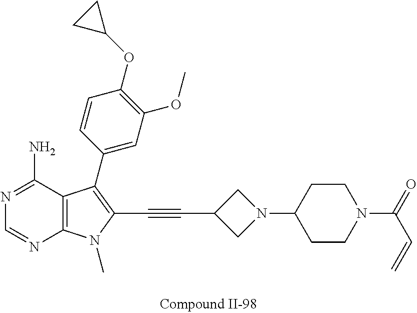 [SMILES: C=CC(=O)N1CCC(N2CC(C#Cc3c(-c4ccc(OC5CC5)c(OC)c4)c4c(N)ncnc4n3C)C2)CC1] | ¹H NMR (400 MHz, DMSO-d₆) δ 8.18 (s, 1H), 7.37 (d, J = 8.2 Hz, 1H), 7.09 − 7.03 (m, 2H), 6.79 (dd, J = 16.7, 10.5 Hz, 1H), 6.28 (s, 1H), 6.07 (dd, J = 16.7, 2.5 Hz, 1H), 5.65 (dd, J = 10.5, 2.4 Hz, 1H), 3.99 (d, J = 13.4 Hz, 1H), 3.91 − 3.83 (m, 1H), 3.81 (s, 1H), 3.79 (s, 3H), 3.72 (s, 3H), 3.57 (t, J = 6.9 Hz, 2H), 3.45 (q, J = 7.4 Hz, 1H), 3.18 (t, J = 11.4 Hz, 1H), 3.02 (t, J = 6.8 Hz, 3H), 2.26 (dt, J = 8.7, 4.7 Hz, 1H), 1.60 (s, 2H), 1.07 (s, 2H), 0.85 − 0.67 (m, 4H). | 527.4 |
| 1-[4-(3-[2-[4- amino-5-(4- cyclopropylphenyl)- 7-methyl- 7H-pyrrolo[2,3- d]pyrimidin-6- yl]ethynyl] azetidin-1- yl)piperidin-1- yl]prop-2-en-1- one | 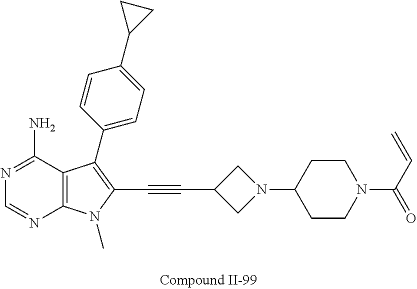 [SMILES: C=CC(=O)N1CCC(N2CC(C#Cc3c(-c4ccc(C5CC5)cc4)c4c(N)ncnc4n3C)C2)CC1] | ¹H NMR (400 MHz, DMSO-d₆) δ 8.19 (s, 1H), 7.40 (d, J = 8.1 Hz, 2H), 7.22 (d, J = 8.1 Hz, 2H), 6.80 (dd, J = 16.7, 10.5 Hz, 1H), 6.08 (dd, J = 16.7, 2.5 Hz, 1H), 5.65 (dd, J = 10.5, 2.5 Hz, 1H), 3.99 (d, J = 13.1 Hz, 1H), 3.83 (d, J = 13.8 Hz, 1H), 3.72 (s, 3H), 3.56 (t, J = 6.9 Hz, 2H), 3.42 (q, J = 7.5 Hz, 1H), 3.20 (t, J = 11.3 Hz, 1H), 3.01 (t, J = 6.9 Hz, 3H), 2.28- 2.26 (dt, J = 8.9, 4.9 Hz, 1H), 2.01- 1.97 (dt, J = 8.8, 5.0 Hz, 1H), 1.61 (m, 2H), 1.08 − 0.98 (m, 4H), 0.77 − 0.73 (m, 2H). | 481.35 |
| 1-(4-(3-((4- amino-5-(4- cyclo- propoxyphenyl)- 7-methyl- 7H-pyrrolo[2,3- d]pyrimidin-6- yl)ethynyl) azetidin-1- yl)piperidin-1- yl)prop-2-en-1- one | 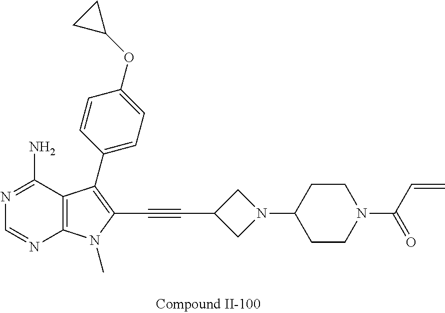 [SMILES: C=CC(=O)N1CCC(N2CC(C#Cc3c(-c4ccc(OC5CC5)cc4)c4c(N)ncnc4n3C)C2)CC1] | ¹H NMR (400 MHz, Chloroform-d) δ 8.36 (s, 1H), 7.52 − 7.45 (m, 2H), 7.22 − 7.14 (m, 2H), 6.58 (dd, J = 16.8, 10.6 Hz, 1H), 6.27 (dd, J = 16.8, 1.9 Hz, 1H), 5.69 (dd, J = 10.5, 2.0 Hz, 1H), 5.30 (s, 1H), 4.25 (s, 1H), 3.96 − 3.76 (m, 5H), 3.72 (t, J = 7.1 Hz, 2H), 3.49 (p, J = 7.8 Hz, 1H), 3.21 (s, 1H), 3.11 (t, J = 7.3 Hz, 3H), 2.40 − 2.17 (m, 1H), 1.80 − 1.67 (m, 2H), 1.32 (ddt, J = 14.4, 9.4, 4.7 Hz, 2H), 0.89 − 0.82 (m, 4H). | 497.4 |
| 1-(4-(3-((4- amino-5-(4- isopropoxy-3- methoxyphenyl)- 7-methyl-7H- pyrrolo[2,3- d]pyrimidin-6- yl)ethynyl) azetidin-1- yl)piperidin-1- yl)prop-2-en-1- one | 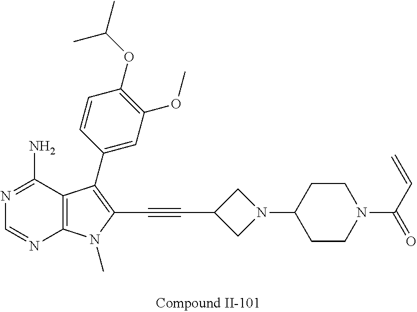 [SMILES: C=CC(=O)N1CCC(N2CC(C#Cc3c(-c4ccc(OC(C)C)c(OC)c4)c4c(N)ncnc4n3C)C2)CC1] | ¹H NMR (400 MHz, DMSO-d₆) δ 8.18 (s, 1H), 7.09 (d, J = 5.8 Hz, 2H), 6.99 (d, J = 8.2 Hz, 1H), 6.78 (dd, J = 16.7, 10.5 Hz, 1H), 6.25 (s, 2H), 6.07 (d, J = 16.6 Hz, 1H), 5.66 (d, J = 10.6 Hz, 1H), 4.61 (p, J = 6.0 Hz, 1H), 3.99 (d, J = 13.1 Hz, 1H), 3.81 (m, 4H), 3.70 (s, 3H), 3.56 (t, J = 6.8 Hz, 2H), 3.45 (q, J = 7.5 Hz, 1H), 3.21 (m, , 1H), 3.00 (t, J = 6.9 Hz, 3H), 2.25 (s, 1H), 1.61 (s, 2H), 1.30 (d, J = 5.9 Hz, 6H), 1.06 (s, 2H). | 529.3 |
| 1-(4-(3-((4- amino-7- methyl-5-(4- (trifluoromethyl) phenyl)-7H- pyrrolo[2,3- d]pyrimidin-6- yl)ethynyl)azetidin- 1- yl)piperidin-1- yl)prop-2-en-1- one | 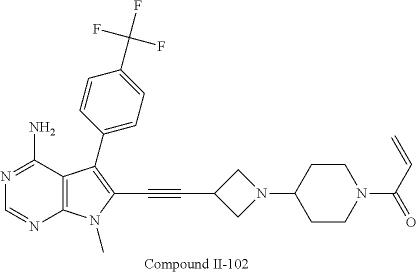 [SMILES: C=CC(=O)N1CCC(N2CC(C#Cc3c(-c4ccc(C(F)(F)F)cc4)c4c(N)ncnc4n3C)C2)CC1] | ¹H NMR (400 MHz, DMSO-d₆) δ 8.22 (s, 1H), 7.85 (d, J = 8.1 Hz, 2H), 7.73 (d, J = 8.0 Hz, 2H), 6.79 (dd, J = 16.7, 10.5 Hz, 1H), 6.30 (br, 2H), 6.07 (dd, J = 16.7, 2.5 Hz, 1H), 5.65 (dd, J = 10.4, 2.5 Hz, 1H), 3.98 (d, J = 12.9 Hz, 1H), 3.82 (d, J = 13.7 Hz, 1H), 3.74 (s, 3H), 3.56 (t, J = 6.9 Hz, 2H), 3.43 (p, J = 7.4 Hz, 1H), 3.18 (t, J = 11.6 Hz, 1H), 3.02 (t, J = 6.8 Hz, 3H), 2.31 − 2.22 (m, 1H), 1.61 (s, 2H), 1.07 (s, 2H). | 509.30 |
| 1-(4-(3-((4- amino-7- methyl-5-(4- (trifluoromethoxy) phenyl)-7H- pyrrolo[2,3- d]pyrimidin-6- yl)ethynyl) azetidin-1- yl)piperidin-1- yl)prop-2-en-1- one | 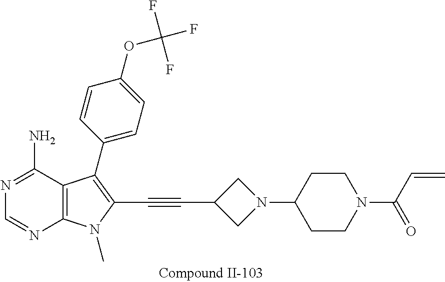 [SMILES: C=CC(=O)N1CCC(N2CC(C#Cc3c(-c4ccc(OC(F)(F)F)cc4)c4c(N)ncnc4n3C)C2)CC1] | ¹H NMR (400 MHz, DMSO-d₆) δ 8.21 (s, 1H), 7.67 − 7.60 (m, 2H), 7.50 (d, J = 8.2 Hz, 2H), 6.80 (dd, J = 16.7, 10.5 Hz, 1H), 6.24 (s, 2H), 6.08 (dd, J = 16.7, 2.4 Hz, 1H), 5.65 (dd, J = 10.4, 2.5 Hz, 1H), 4.01 (s, 1H), 3.86 (s, 1H), 3.74 (s, 3H), 3.52 (d, 2H), 3.42 (d, 1H), 3.16 (s, 2H), 2.98 (s, 2H), 2.29 (s, 1H), 1.65 (s, 2H), 1.09 (s, 2H). | 525.30 |
| 1-[4-(3-[2-[4- amino-5-(2,3- dihydro-1,4- benzodioxin-6- yl)-7-methyl- 7H-pyrrolo[2,3- d]pyrimidin-6- yl]ethynyl] azetidin-1- yl)piperidin-1- yl]prop-2-en-1- one | 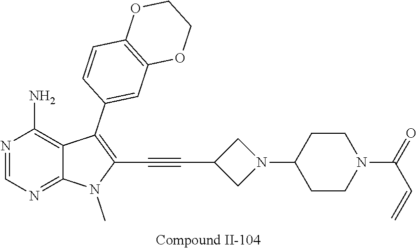 [SMILES: C=CC(=O)N1CCC(N2CC(C#Cc3c(-c4ccc5c(c4)OCCO5)c4c(N)ncnc4n3C)C2)CC1] | 1H NMR (400 MHz, DMSO-d6) δ 8.18 (s, 1H), 7.02 − 6.94 (m, 3H), 6.80 (dd, J = 16.7, 10.5 Hz, 1H), 6.08 (dd, J = 16.7, 2.5 Hz, 1H), 5.66 (dd, J = 10.5, 2.5 Hz, 1H), 4.31 (s, 4H), 3.98 (d, 1H), 3.83 (d, J = 12.8 Hz, 1H), 3.71 (s, 3H), 3.58 (t, J = 6.9 Hz, 2H), 3.44 (t, J = 7.5 Hz, 1H), 3.19 (m, J = 11.8 Hz, 1H), 3.03 (t, J = 7.0 Hz, 3H), 2.30-2.27 (m, 1H), 1.65-1.62 (m, 2H), 1.09 (m, 2H). | 499.30 |
| 1-(4-(3-((4- amino-7- methyl-5-(4- methyl-3,4- dihydro-2H- benzo[b][1,4] oxazin-7-yl)-7H- pyrrolo[2,3- d]pyrimidin-6- yl)ethynyl) azetidin-1- yl)piperidin-1- yl)prop-2-en-1- one | 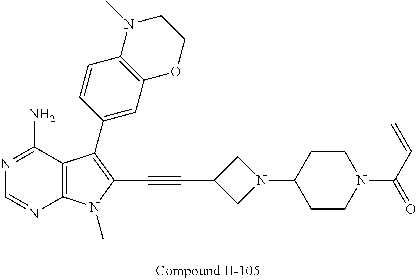 [SMILES: C=CC(=O)N1CCC(N2CC(C#Cc3c(-c4ccc5c(c4)OCCN5C)c4c(N)ncnc4n3C)C2)CC1] | ¹H NMR (400 MHz, DMSO-d₆) δ 8.16 (s, 1H), 6.94 − 6.85 (m, 2H), 6.85 − 6.74 (m, 2H), 6.07 (d, J = 16.7 Hz, 1H), 5.95(s, 2H), 5.65 (d, J = 10.4 Hz, 1H), 4.27 (d, J = 4.6 Hz, 2H), 3.98 (d, J = 12.9 Hz, 1H), 3.83 (d, J = 13.2 Hz, 1H), 3.69 (s, 3H), 3.57 (t, J = 6.9 Hz, 2H), 3.43 (q, J = 7.6 Hz, 1H), 3.33 − 3.26 (m, 2H), 3.20 (t, J = 11.6 Hz, 1H), 3.02 (q, J = 11.2, 8.9 Hz, 3H), 2.89 (s, 3H), 2.29 (s, 1H), 1.62 (s, 2H), 1.08 (s, 2H). | 512.2 |
| 1-(4-(3-((4- amino-5-(3,4- dimethoxyphenyl)- 7-methyl-7H- pyrrolo[2,3- d]pyrimidin-6- yl)ethynyl) azetidin-1- yl)piperidin-1- yl)prop-2-en-1- one | 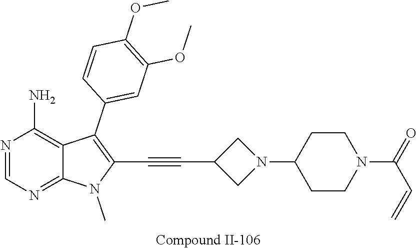 [SMILES: C=CC(=O)N1CCC(N2CC(C#Cc3c(-c4ccc(OC)c(OC)c4)c4c(N)ncnc4n3C)C2)CC1] | ¹H NMR (400 MHz, DMSO-d₆) δ 8.18 (s, 1H), 7.10 (dd, J = 5.2, 3.1 Hz, 2H), 7.02 (dd, J = 8.0, 2.0 Hz, 1H), 6.79 (dd, J = 16.7, 10.5 Hz, 1H), 6.21 (s, 1H), 6.07 (dd, J = 16.8, 2.5 Hz, 1H), 5.65 (dd, J = 10.4, 2.5 Hz, 1H), 3.98 (s, 1H), 3.81 (d, J = 4.5 Hz, 6H), 3.72 (s, 3H), 3.56 (t, J = 7.0 Hz, 2H), 3.44 (p, J = 7.5 Hz, 1H), 3.18 (t, J = 11.4 Hz, 1H), 3.01 (t, J = 6.9 Hz, 3H), 2.26 (s, 1H), 2.08 (s, 1H), 1.61 (s, 2H), 1.05 (d, J = 14.5 Hz, 2H). | 501.3 |
| 1-(4-(3-((4- amino-7- methyl-5-(4- methyl-3,4- dihydro-2H- benzo[b][1,4] oxazin-6-yl)-7H- pyrrolo[2,3- d]pyrimidin-6- yl)ethynyl) azetidin-1- yl)piperidin-1- yl)prop-2-en-1- one | 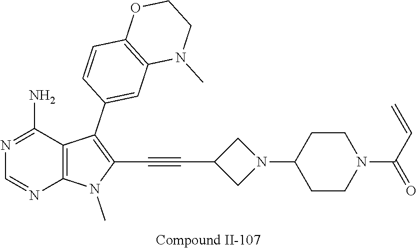 [SMILES: C=CC(=O)N1CCC(N2CC(C#Cc3c(-c4ccc5c(c4)N(C)CCO5)c4c(N)ncnc4n3C)C2)CC1] | ¹H NMR (400 MHz, DMSO-d₆) δ 8.16 (s, 1H), 6.81 (q, J = 9.9, 9.4 Hz, 3H), 6.69 (d, J = 8.1 Hz, 1H), 6.07 (d, J = 16.6 Hz, 1H), 6.07 (d, J = 16.6 Hz, 1H), 5.65 (d, J = 10.5 Hz, 1H), 4.28 (s, 2H), 3.99 (d, J = 12.6 Hz, 1H), 3.83 (d, J = 13.5 Hz, 1H), 3.70 (s, 3H), 3.58 (s, 2H), 3.43 (d, 1H), 3.34 (s, 2H), 3.29 (s, 2H), 3.23 (d, 1H), 3.05 − 2.98 (m, 3H), 2.87 (s, 3H), 2.29 (s, 1H), 1.62 (s, 2H), 1.07 (s, 2H). | 512.3 |
| 1-((3R,4S)-4-(3- ((4-amino-5- (benzo[d][1,3] dioxol-4-yl)-7- isopropyl-7H- pyrrolo[2,3- d]pyrimidin-6- yl)ethynyl)azetidin- 1-yl)-3- hydroxypiperidin- 1-yl)prop-2- en-1-one | 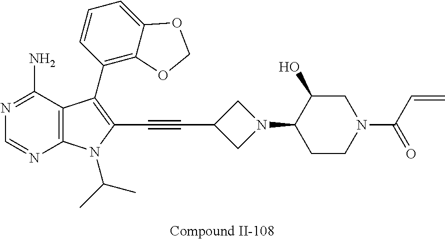 [SMILES: C=CC(=O)N1CC[C@@H](N2CC(C#Cc3c(-c4cccc5c4OCO5)c4c(N)ncnc4n3C(C)C)C2)[C@@H](O)C1] | ¹H NMR (400 MHz, DMSO-d₆) δ 8.15 (s, 1H), 6.97-6.96(d, J = 6.0 Hz, 2H), 6.94 − 6.89 (m, 1H), 6.76-6.74 (td, J = 19.0, 16.8, 10.6 Hz, 1H), 6.69-6.04 (d, J = 12.3 Hz, 5H), 5.64- 5.61 (d, J = 10.3 Hz, 1H), 5.11 − 5.08 (m, 1H), 4.56-4.42 (d, J = 3.5 Hz, 1H), 4.00-3.77 (dd, J = 12.9 Hz, 1H), 3.63 − 3.56 (m, 4H), 3.41 (t, J = 7.6 Hz, 1H), 3.22-3.19 (d, J = 13.4 Hz, 1H), 3.05-3.01 (q, J = 8.4, 7.1 Hz, 2H), 2.94 − 2.92 (m, 1H), 2.34- 2.25 (d, 1H), 1.62 (d, J = 6.9 Hz, 6H), 1.50 − 1.37 (d, 2H). | 529.2 |
| 1-(4-(3-((4- amino-5- (benzo[d][1,3] dioxol-4-yl)-7- isopropyl-7H- pyrrolo[2,3- d]pyrimidin-6- yl)ethynyl) azetidin-1- yl)piperidin-1- yl)prop-2-en-1- one | 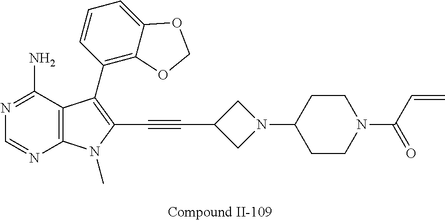 [SMILES: C=CC(=O)N1CCC(N2CC(C#Cc3c(-c4cccc5c4OCO5)c4c(N)ncnc4n3C)C2)CC1] | ¹H NMR (400 MHz, Methanol-d₄) δ 8.17 (s, 1H), 7.05 − 6.97 (m, 3H), 6.82 − 6.75 (m, 1H), 6.20 (dd, J = 16.8, 1.9 Hz, 1H), 6.03 (d, J = 11.2 Hz, 2H), 5.76 (dd, J = 10.7, 2.0 Hz, 1H), 5.19 (p, J = 6.9 Hz, 1H), 4.48 (d, J = 13.4 Hz, 1H), 4.12 (d, J = 14.1 Hz, 1H), 3.96 (s, 2H), 3.70 (t, J = 8.0 Hz, 1H), 3.55 (s, 2H), 3.20 (t, J = 12.4 Hz, 1H), 2.88 (t, J = 12.2 Hz, 1H), 2.78 (s, 1H), 1.90 (d, J = 12.6 Hz, 2H), 1.75 (d, J = 6.9 Hz, 6H), 1.25 (t, J = 11.7 Hz, 2H). | 513.3 |
| 1-((3R,4S)-4-(3- ((4-amino-5- (benzo[d][1,3] dioxol-4-yl)-7- isopropyl-7H- pyrrolo[2,3- d]pyrimidin-6- yl)ethynyl) azetidin-1-yl)-3- fluoropiperidin- 1-yl)prop-2-en- 1-one | 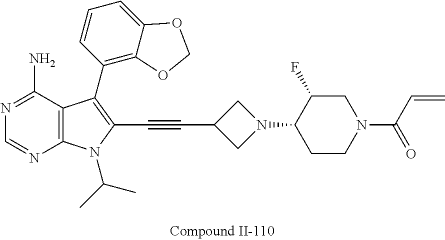 [SMILES: C=CC(=O)N1CC[C@H](N2CC(C#Cc3c(-c4cccc5c4OCO5)c4c(N)ncnc4n3C(C)C)C2)[C@H](F)C1] | ¹H NMR (400 MHz, DMSO-d₆) δ 8.15 (s, 1H), 7.01 (m, 2H), 6.91 (m, 1H), 6.78 (m, 1H), 6.28 (s, 1H), 6.14 − 6.02 (m, 3H), 5.67 (d, J = 10.4 Hz, 1H), 5.09 (p, J = 7.0 Hz, 1H), 4.72 (d, 1H), 4.37 − 3.88 (m, 2H), 3.60 − 3.52 (m, 2H), 3.51 − 3.38 (m, 1H), 3.17 (t, J = 12.8 Hz, 1H), 3.11 − 3.02 (m, 2H), 2.81 (t, J = 12.2 Hz, 1H), 2.39 (m, 1H), 1.62 (d, J = 6.8 Hz, 6H), 1.52 (d, 1H), 1.31 (s, 1H). | 531.2 |
| 1-((3R,4S)-4-(3- ((4-amino-5-(4- chloro-3- methylphenyl)- 7-isopropyl-7H- pyrrolo[2,3- d]pyrimidin-6- yl)ethynyl)azetidin- 1-yl)-3- fluoropiperidin- 1-yl)prop-2-en- 1-one | 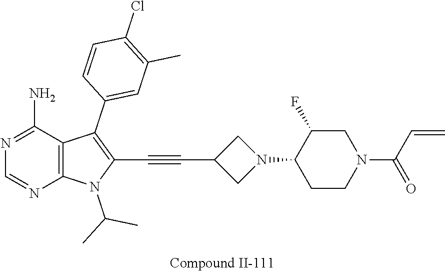 [SMILES: C=CC(=O)N1CC[C@H](N2CC(C#Cc3c(-c4ccc(Cl)c(C)c4)c4c(N)ncnc4n3C(C)C)C2)[C@H](F)C1] | ¹H NMR (400 MHz, DMSO-d₆) δ 8.17 (s, 1H), 7.56 − 7.48 (m, 2H), 7.32 (d, J = 8.2 Hz, 1H), 6.80 (ddd, J = 22.8, 16.5, 10.5 Hz, 1H), 6.25 (s, 1H),6.14 − 6.05 (m, 1H), 5.68 (d, J = 10.6 Hz, 1H), 5.10 (p, J = 6.9 Hz, 1H), 4.73 (d, 1H), 4.36 − 3.89 (m, 2H), 3.62 − 3.54 (m, 2H), 3.46 (dq, J = 15.4, 8.2, 7.8 Hz,1H), 3.28 − 3.06 (m, 3H), 2.82 (t, J = 12.2 Hz, 1H), 2.40 (s, 4H), 1.62 (d, J = 6.9 Hz, 6H), 1.53 (s, 1H), 1.42 − 1.13 (m, J = 12.2 Hz, 1H). | 535.2 |
| 1-((3R,4S)-4-(3- ((4-amino-5-(4- chloro-3- methylphenyl)- 7-isopropyl-7H- pyrrolo[2,3- d]pyrimidin-6- yl)ethynyl)azetidin- 1-yl)-3- hydroxypiperidin- 1-yl)prop-2- en-1-one | 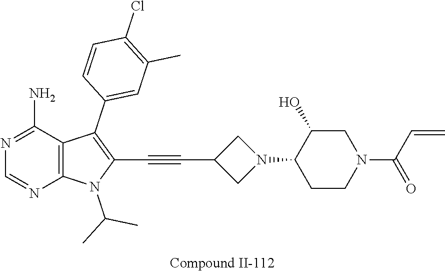 [SMILES: C=CC(=O)N1CC[C@H](N2CC(C#Cc3c(-c4ccc(Cl)c(C)c4)c4c(N)ncnc4n3C(C)C)C2)[C@H](O)C1] | ¹H NMR (400 MHz, Methanol-d₄) δ 8.16 (s, 1H), 7.53 − 7.49 (m, 2H), 7.37-7.35 (d, J = 8.2 Hz, 1H), 6.79 − 6.72 (m, 1H), 6.19-6.15 (d, J = 16.9 Hz, 1H), 5.75-5.72 (t, J = 9.7 Hz, 1H), 5.22-5.16 (p, J = 7.0 Hz, 1H), 4.47-4.36 (dd, J = 26.9, 12.3 Hz, 1H), 4.08-3.97 (dd, J = 28.8, 14.2 Hz, 1H), 3.80 (d, J = 11.5 Hz, 1H), 3.75 (d, J = 7.5 Hz, 1H), 3.75 − 3.64 (m, 1H), 3.50 (q, J = 7.8 Hz, 1H), 3.25 (d, J = 14.3 Hz, 1H), 3.16 (d, J = 6.8 Hz, 2H),3.06-2.84(m,1H), 2.48 (s, 3H), 2.38 (d, J = 7.5 Hz, 1H), 1.70 (d, J = 6.9 Hz, 6H), 1.56 (s, 2H). | 533.3 |
| 1-((3R,4S)-4-(3- ((4-amino-5-(4- chloro-3- methylphenyl)- 7-ethyl-7H- pyrrolo[2,3- d]pyrimidin-6- yl)ethynyl) azetidin-1-yl)-3- hydroxypiperidin- 1-yl)prop-2- en-1-one | 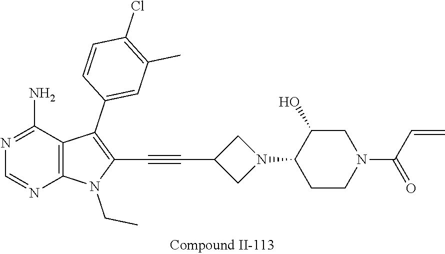 [SMILES: C=CC(=O)N1CC[C@H](N2CC(C#Cc3c(-c4ccc(Cl)c(C)c4)c4c(N)ncnc4n3CC)C2)[C@H](O)C1] | ¹H NMR (400 MHz, DMSO-d₆) δ 8.19 (s, 1H), 7.55 − 7.51 (m, 2H), 7.34 (dd, J = 8.2, 2.2 Hz, 1H), 6.75 (ddd, J = 20.6, 16.7, 10.5 Hz, 1H), 6.22 (s, 1H), 6.06 (dt, J = 16.8, 2.3 Hz, 1H), 5.63 (ddd, J = 10.5, 5.1, 2.5 Hz, 1H), 4.56 − 4.43 (m, 1H), 4.29 (q, J = 7.1 Hz, 2H), 4.29 − 4.23 (m, 1H), 3.79 − 3.78 (m, 4H), 3.45 (q, J = 7.6 Hz, 1H), 3.29 − 3.16 (m, 1H), 3.08 (q, J = 8.1, 6.9 Hz, 2H), 2.98 − 2.84 (m, 1H), 2.41 (s, 4H), 2.08 (s, 1H), 1.39 − 1.32 (m, 5H). | 519.3 |
| 1-(4-(3-((4- amino-5-(4- chloro-3- methylphenyl)- 7-isopropyl-7H- pyrrolo[2,3- d]pyrimidin-6- yl)ethynyl) azetidin-1- yl)piperidin-1- yl)prop-2-en-1- one | 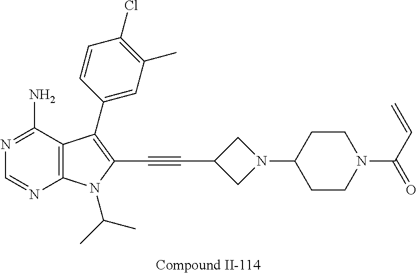 [SMILES: C=CC(=O)N1CCC(N2CC(C#Cc3c(-c4ccc(Cl)c(C)c4)c4c(N)ncnc4n3C(C)C)C2)CC1] | ¹H NMR (400 MHz, DMSO-d₆) δ 8.17 (s, 1H), 7.54 − 7.50 (m, 2H), 7.32 (d, J = 8.4 Hz, 1H), 6.83 − 6.76 (dd, J = 16.7, 10.4 Hz, 1H), 6.07 (d, J = 16.8 Hz, 2H), 5.67 − 5.64 (d, J = 10.4 Hz, 1H), 5.13 − 5.06 (m, 1H), 3.97 (d, J = 13.1 Hz, 1H), 3.85 (d, J = 13.1 Hz, 1H), 3.54 (t, J = 6.9 Hz, 2H), 3.41 (t, J = 7.5 Hz, 1H), 3.21 (dd, J = 15.5, 7.9 Hz, 1H), 2.99 (t, J = 6.7 Hz, 3H), 2.42 (s, 3H), 2.24 (s, 1H), 1.62 (d, J = 6.9 Hz, 8H), 1.12 (s, 2H). | 517.2 |
| (E)-1- ((1R,5S,6s)-6- ((4-amino-5- (benzo[d][1,3] dioxol-4-yl)-7- methyl-7H- pyrrolo[2,3- d]pyrimidin-6- yl)ethynyl)-3- azabicyclo[3.1.0] hexan-3-yl)-4- (dimethylamino) but-2-en-1-one | 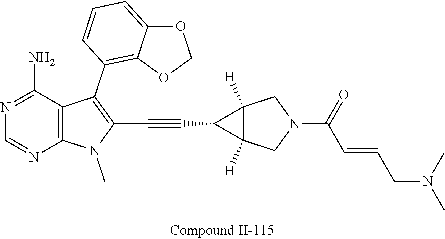 [SMILES: CN(C)C/C=C/C(=O)N1C[C@@H]2[C@@H](C#Cc3c(-c4cccc5c4OCO5)c4c(N)ncnc4n3C)[C@@H]2C1] | ¹H NMR (400 MHz, DMSO-d₆) δ 8.16 (s, 1H), 7.01 − 6.92 (m, 2H), 6.91 − 6.82 (m, 1H), 6.58 (dt, J = 15.2, 6.2 Hz, 1H), 6.28 (dt, J = 15.1, 1.6 Hz, 1H), 6.17 (s, 2H), 6.04 (s, 2H), 3.85 (d, J = 10.8 Hz, 1H), 3.73 (d, J = 12.5 Hz, 1H), 3.69 (s, 3H), 3.62 (dd, J = 10.7, 4.3 Hz, 1H), 3.38 (dd, J = 12.5, 4.3 Hz, 1H), 3.00 (dd, J = 6.2, 1.5 Hz, 2H), 2.13 (s, 6H), 2.06 (dt, J = 7.4, 3.7 Hz, 1H), 2.00 (dt, J = 7.6, 3.9 Hz, 1H), 1.49 (t, J = 3.4 Hz, 1H). | 485.2 |
| (E)-1- ((1R,5S,6s)-6- ((4-amino-5- (benzo[d][1,3] dioxol-4-yl)-7- isopropyl-7H- pyrrolo[2,3- d]pyrimidin-6- yl)ethynyl)-3- azabicyclo[3.1.0] hexan-3-yl)-4- (dimethylamino) but-2-en-1-one | 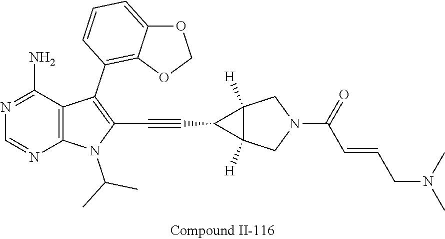 [SMILES: CC(C)n1c(C#C[C@@H]2[C@H]3CN(C(=O)/C=C/CN(C)C)C[C@@H]23)c(-c2cccc3c2OCO3)c2c(N)ncnc21] | ¹H NMR (400 MHz, DMSO-d₆) δ 8.15 (d, J = 5.7 Hz, 1H), 7.00 − 6.92 (m, 2H), 6.87 (dd, J = 6.4, 2.7 Hz, 1H), 6.58 (dt, J = 15.1, 6.3 Hz, 1H), 6.29 (d, J = 15.3 Hz, 1H), 6.16 (s, 1H), 6.04 (d, J = 4.5 Hz, 1H), 5.05 (p, J = 6.9 Hz, 1H), 3.85 (d, J = 10.7 Hz, 1H), 3.73 (d, J = 12.5 Hz, 1H), 3.62 (d, J = 9.4 Hz, 1H), 3.4 (d, 1H), 3.04 (d, J = 6.2 Hz, 2H), 2.16 (s, 6H), 2.02 (s, 1H), 1.96 (s, 1H), 1.60 (d, J = 6.9 Hz, 6H), 1.49 (t, J = 3.4 Hz, 1H). | 513.3 |
| (E)-1- ((1S,5R,6s)-6- ((4-amino-5- (benzo[d][1,3] dioxol-4-yl)-7- ethyl-7H- pyrrolo[2,3- d]pyrimidin-6- yl)ethynyl)-3- aza- bicyclo[3.1.0] hexan-3-yl)-4- (dimethylamino) but-2-en-1-one | 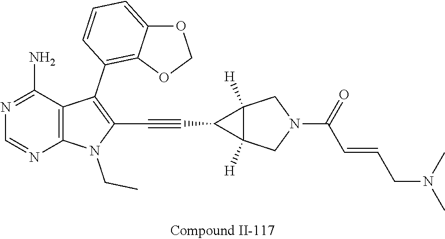 [SMILES: CCn1c(C#C[C@@H]2[C@H]3CN(C(=O)/C=C/CN(C)C)C[C@@H]23)c(-c2cccc3c2OCO3)c2c(N)ncnc21] | ¹H NMR (400 MHz, DMSO-d₆) δ 8.16 (s, 1H), 6.98 − 6.90 (m, 2H), 6.87 (dd, J = 5.8, 3.4 Hz, 1H), 6.58 (dt, J = 15.2, 6.2 Hz, 1H), 6.43 (s, 1H), 6.29 (d, J = 15.2 Hz, 1H), 6.05 (s, 2H), 4.22 (q, J = 7.2 Hz, 2H), 3.85 (d, J = 10.7 Hz, 1H), 3.74 (d, J = 12.5 Hz, 1H), 3.62 (dd, J = 10.7, 4.2 Hz, 1H), 3.42 − 3.34 (m, 1H), 3.03 (d, J = 6.2 Hz, 2H), 2.15 (s, 6H), 2.01 (ddt, J = 27.3, 7.5, 3.8 Hz, 2H), 1.49 (t, J = 3.4 Hz, 1H), 1.31 (t, J = 7.1 Hz, 3H). | 499.3 |
| 1-((3R,4S)-4-(3- ((4-amino-5-(4- chloro-3- (difluoromethyl) phenyl)-7- isopropyl-7H- pyrrolo[2,3- d]pyrimidin-6- yl)ethynyl)azetidin- 1-yl)-3- hydroxypiperidin- 1-yl)prop-2- en-1-one | 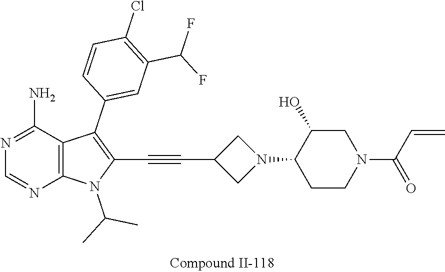 [SMILES: C=CC(=O)N1CC[C@H](N2CC(C#Cc3c(-c4ccc(Cl)c(C(F)F)c4)c4c(N)ncnc4n3C(C)C)C2)[C@H](O)C1] | ¹H NMR (400 MHz, DMSO-d₆) δ 8.19 (s, 1H), 7.83 (s, 1H), 7.71 (d, J = 8.3 Hz, 1H), 7.66 − 7.60 (m, 1H), 7.42 − 7.15 (t, 1H), 6.76 (m, 1H), 6.31 (s, 2H), 6.05 (dd, 1H), 5.63 (dd, J = 10.5, 2.5 Hz, 1H), 5.12 (dd, J = 13.6, 6.9 Hz, 1H), 4.54 − 4.41 (d, J = 3.1 Hz, 1H), 4.01 − 3.80 (dd, 1H), 3.75 − 3.51 (m, 4H), 3.50 − 3.35 (m, 1H), 3.20 (d, J = 13.3 Hz, 1H), 3.06 (m, 3H), 2.27 (dt, 1H), 1.62 (d, J = 6.9 Hz, 6H), 1.37 (d, J = 5.9 Hz, 2H). | 569.2 |
| 1-(4-(3-((4- amino-5- (benzo[d][1,3] dioxol-4-yl)-7- ethyl-7H- pyrrolo[2,3- d]pyrimidin-6- yl)ethynyl) azetidin-1- yl)piperidin-1- yl)prop-2-en-1- one | 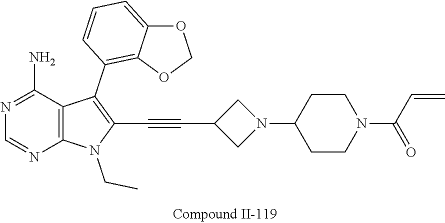 [SMILES: C=CC(=O)N1CCC(N2CC(C#Cc3c(-c4cccc5c4OCO5)c4c(N)ncnc4n3CC)C2)CC1] | ¹H NMR (400 MHz, DMSO-d₆) δ 8.17 (s, 1H), 6.97 (q, J = 3.9, 3.2 Hz, 2H), 6.90 (dd, J = 6.2, 2.9 Hz, 1H), 6.79 (dd, J = 16.7, 10.5 Hz, 1H), 6.23 (br, 1H), 6.05 (s, 3H), 5.65 (dd, J = 10.5, 2.5 Hz, 1H), 4.25 (q, J = 7.1 Hz, 2H), 3.98 (d, J = 13.0 Hz, 1H), 3.81 (d, J = 13.3 Hz, 1H), 3.55 (t, J = 7.0 Hz, 2H), 3.41 (p, J = 7.5 Hz, 1H), 3.19 (t, J = 11.5 Hz, 1H), 2.97 (q, J = 7.3 Hz, 3H), 2.24 (dt, J = 8.8, 4.7 Hz, 1H), 1.60 (s, 2H), 1.33 (t, J = 7.1 Hz, 3H), 1.08 (d, J = 13.0 Hz, 2H). | 499.4 |
| 1-(4-(3-((4- amino-5-(7- fluorobenzo[d] [1,3]dioxol-4- yl)-7-methyl- 7H-pyrrolo[2,3- d]pyrimidin-6- yl)ethynyl)azeti din-1- yl)piperidin-1- yl)prop-2-en-1- one | 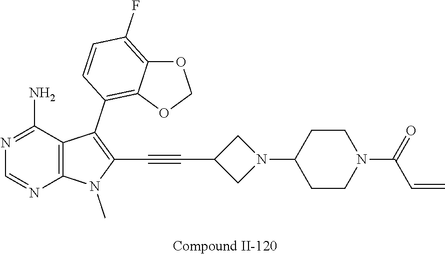 [SMILES: C=CC(=O)N1CCC(N2CC(C#Cc3c(-c4ccc(F)c5c4OCO5)c4c(N)ncnc4n3C)C2)CC1] | ¹H NMR (400 MHz, DMSO-d₆) δ 8.16 (s, 1H), 6.96 (dd, J = 10.0, 8.8 Hz, 1H), 6.88 (dd, J = 8.8, 5.0 Hz, 1H), 6.79 (dd, J = 16.7, 10.5 Hz, 1H), 6.28 (s, 2H), 6.14 (s, 2H), 6.07 (dd, J = 16.7, 2.4 Hz, 1H), 5.65 (dd, J = 10.5, 2.5 Hz, 1H), 3.99 (d, J = 12.7 Hz, 1H), 3.82 (d, J = 13.2 Hz, 1H), 3.71 (s, 3H), 3.55 (t, J = 6.9 Hz, 2H), 3.43 (q, J = 7.4 Hz, 1H), 3.18 (t, J = 11.7 Hz, 1H), 2.98 (q, J = 8.5, 6.9 Hz, 3H), 2.25 (p, J = 4.7 Hz, 1H), 1.61 (s, 2H), 1.06 (s, 2H). | 503.2 |
| 1-(4-(3-((4- amino-7-ethyl- 5-(3-methoxy-4- (6- methylpyridin- 2- yloxy)phenyl)- 7H-pyrrolo[2,3- d]pyrimidin-6- yl)ethynyl) azetidin-1- yl)piperidin-1- yl)prop-2-en-1- one | 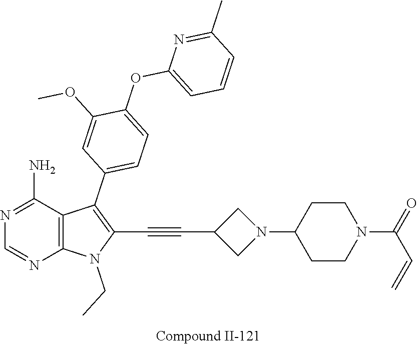 [SMILES: C=CC(=O)N1CCC(N2CC(C#Cc3c(-c4ccc(Oc5cccc(C)n5)c(OC)c4)c4c(N)ncnc4n3CC)C2)CC1] | ¹H NMR (400 MHz, DMSO-d₆) δ 8.20 (s, 1H), 7.68 (dd, J = 8.2, 7.3 Hz, 1H), 7.27 (d, J = 2.0 Hz, 1H), 7.24 (d, J = 8.1 Hz, 1H), 7.12 (dd, J = 8.1, 1.9 Hz, 1H), 6.95 (d, J = 7.3 Hz, 1H), 6.82 − 6.70 (m, 2H), 6.32 (s, 1H), 6.06 (dd, J = 16.7, 2.5 Hz, 1H), 5.64 (dd, J = 10.4, 2.5 Hz, 1H), 4.28 (q, J = 7.0 Hz, 2H), 3.96 (d, J = 13.1 Hz, 1H), 3.76 (s, 4H), 3.57 (t, J = 6.8 Hz, 2H), 3.47 (p, J = 7.3 Hz, 1H), 3.14 (t, J = 11.7 Hz, 1H), 3.02 (t, J = 6.5 Hz, 2H), 2.95 (t, J = 10.6 Hz, 1H), 2.32 (s, 3H), 2.24 (p, J = 4.7 Hz, 1H), 1.58 (s, 2H), 1.35 (t, J = 7.1 Hz, 3H), 1.05 (s, 2H). | 592.40 |
| 1-(4-(3-((4- amino-5-(3- methoxy-4-(6- methylpyridin-2- yloxy)phenyl)- 7-(2- methoxyethyl)- 7H-pyrrolo[2,3- d]pyrimidin-6- yl)ethynyl) azetidin-1- yl)piperidin-1- yl)prop-2-en-1- one | 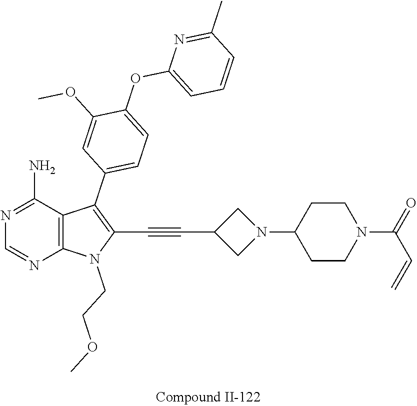 [SMILES: C=CC(=O)N1CCC(N2CC(C#Cc3c(-c4ccc(Oc5cccc(C)n5)c(OC)c4)c4c(N)ncnc4n3CCOC)C2)CC1] | ¹H NMR (400 MHz, DMSO-d₆) δ 8.20 (s, 1H), 7.72 − 7.65 (m, 1H), 7.29 − 7.21 (m, 2H), 7.12 (dd, J = 8.1, 2.0 Hz, 1H), 6.95 (d, J = 7.3 Hz, 1H), 6.81 − 6.70 (m, 2H), 6.32 (s, 1H), 6.06 (dd, J = 16.7, 2.5 Hz, 1H), 5.64 (dd, J = 10.5, 2.5 Hz, 1H), 4.40 (t, J = 5.9 Hz, 2H), 3.96 (d, J = 13.1 Hz, 1H), 3.75 (d, J = 2.4 Hz, 6H), 3.56 (t, J = 6.8 Hz, 2H), 3.46 (p, J = 7.2 Hz, 1H), 3.26 (d, J = 2.8 Hz, 3H), 3.14 (t, J = 11.4 Hz, 1H), 3.02 (t, J = 6.6 Hz, 2H), 2.95 (t, J = 11.1 Hz, 1H), 2.32 (s, 3H), 2.24 (dt, J = 8.8, 4.8 Hz, 1H), 1.58 (s, 2H), 1.05 (s, 2H). | 622.45 |
| 1-((3R,4S)-4-(3- ((4-amino-5-(3- methoxy-4-(6- methylpyridin- 2- yloxy)phenyl)- 7-(2- methoxyethyl)- 7H-pyrrolo[2,3- d]pyrimidin-6- yl)ethynyl)azetidin- 1-yl)-3- fluoropiperidin- 1-yl)prop-2-en- 1-one | 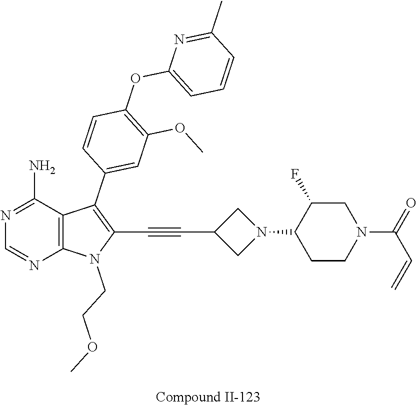 | ¹H NMR (400 MHz, DMSO-d₆) δ 8.20 (s, 1H), 7.72 − 7.65 (m, 1H), 7.28 − 7.22 (m, 2H), 7.12 (dd, J = 8.1, 1.9 Hz, 1H), 6.96 (d, J = 7.3 Hz, 1H), 6.85 − 6.70 (m, 2H), 6.32 (s, 1H), 6.08 (dd, J = 16.6, 2.4 Hz, 1H), 5.67 (d, J = 10.3 Hz, 1H), 4.64 (d, J = 49.0 Hz, 1H), 4.40 (t, J = 5.8 Hz, 2H), 4.26 − 4.10 (m, 1H), 3.88 (d, J = 14.0 Hz, 1H), 3.75 (s, 5H), 3.65 − 3.56 (m, 2H), 3.52 (q, J = 7.1 Hz, 1H), 3.33(dd, J = 14.6, 8.0 Hz, 3H), 3.11(m,3H),2.76 (t, J = 12.3 Hz, 1H), 2.39 (d, J = 11.4 Hz, 1H), 2.33 (s, 3H), 1.51 (d, J = 7.4 Hz, 1H), 1.35 (dd, J = 46.9, 10.8 Hz, 1H). | 640.45 |
| N-((1r,4r)-4-((4- amino-5-(3- methoxy-4-((6- methylpyridin- 2- yl)oxy)phenyl)- 7- (tetrahydrofuran- 3-yl)-7H- pyrrolo[2,3- d]pyrimidin-6- yl)ethynyl) cyclohexyl) acrylamide | 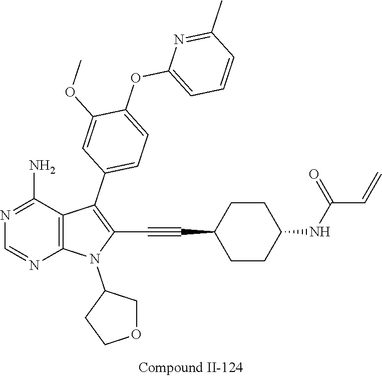 [SMILES: C=CC(=O)N[C@H]1CC[C@H](C#Cc2c(-c3ccc(Oc4cccc(C)n4)c(OC)c3)c3c(N)ncnc3n2C2CCOC2)CC1] | ¹H NMR (400 MHz, DMSO-d₆) δ 8.19 (s, 1H), 7.97 (d, J = 7.7 Hz, 1H), 7.68 (t, J = 7.8 Hz, 1H), 7.26 − 7.19 (m, 2H), 7.09 (dd, J = 8.1, 2.0 Hz, 1H), 6.94 (d, J = 7.3 Hz, 1H), 6.71 (d, J = 8.2 Hz, 1H), 6.20 (dd, J = 17.0, 10.0 Hz, 1H), 6.10 − 6.02 (m, 1H), 5.59 − 5.49 (m, 2H), 4.22 (d, J = 6.8 Hz, 1H), 4.08 (p, J = 8.4 Hz, 2H), 3.98 − 3.90 (m, 1H), 3.74 (s, 3H), 3.61 (s, 1H), 2.61 (t, J = 11.7 Hz, 2H), 2.36 (m, 1H), 2.31 (s, 3H), 1.97 (d, J = 12.7 Hz, 2H), 1.81 (d, J = 12.5 Hz, 2H), 1.47 (m, 2H), 1.25 (m, 2H). | 593.40 |
| 1-(4-(3-((4- amino-5-(3- methoxy-4-(6- methylpyridin- 2- yloxy)phenyl)- 7- (tetrahydrofuran- 3-yl)-7H- pyrrolo[2,3- d]pyrimidin-6- yl)ethynyl) azetidin-1- yl)piperidin-1- yl)prop-2-en-1- one | 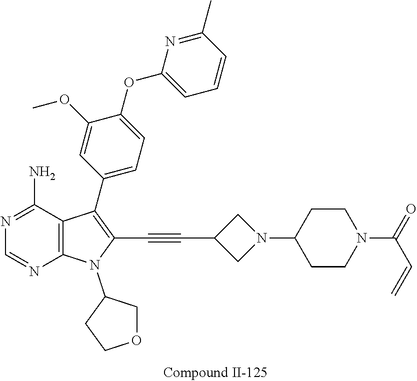 [SMILES: C=CC(=O)N1CCC(N2CC(C#Cc3c(-c4ccc(Oc5cccc(C)n5)c(OC)c4)c4c(N)ncnc4n3C3CCOC3)C2)CC1] | ¹H NMR (400 MHz, Chloroform-d) δ 8.34 (s, 1H), 7.59 (t, J = 7.7 Hz, 1H), 7.28 − 7.18 (m, 2H), 7.14 (dd, J = 8.1, 2.0 Hz, 1H), 6.90 (d, J = 7.3 Hz, 1H), 6.68 (d, J = 8.2 Hz, 1H), 6.58 (dd, J = 16.8, 10.6 Hz, 1H), 6.27 (dd, J = 16.8, 1.9 Hz, 1H), 5.72 − 5.60 (m, 2H), 5.48 (s, 2H), 4.38 (td, J = 8.3, 5.4 Hz, 1H), 4.35 − 4.15 (m, 3H), 4.05 (q, J = 7.6 Hz, 1H), 3.84 (s, 4H), 3.68 (t, J = 7.1 Hz, 2H), 3.54 − 3.42 (m, 1H), 3.23 − 2.97 (m, 4H), 2.78 (dq, J = 14.6, 7.2 Hz, 1H), 2.46 (s, 4H), 2.32 (dt, J = 9.0, 4.8 Hz, 1H), 1.74 − 1.66 (m, 2H), 1.27 (dtd, J = 13.2, 9.4, 3.9 Hz, 2H). | 634.3 |
| 1-((1R,5S,6s)-6- ((4-amino-5- (benzo[d][1,3] dioxol-4-yl)-7- methyl-7H- pyrrolo[2,3- d]pyrimidin-6- yl)ethynyl)-3- azabicyclo[3.1.0] hexan-3- yl)prop-2-en-1- one | 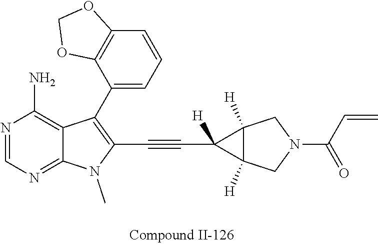 [SMILES: C=CC(=O)N1C[C@@H]2[C@@H](C#Cc3c(-c4cccc5c4OCO5)c4c(N)ncnc4n3C)[C@@H]2C1] | ¹H NMR (500 MHz, DMSO-d₆,): δ = 8.15 (s, 1H), 6.92-7.00 (m, 2H), 6.82- 6.89 (m, 1H), 6.48 (dd, J = 16.7, 10.2 Hz, 1H), 6.09 (dd, J = 16.9, 2.3 Hz, 1H), 6.03 (s, 2H), 5.60-5.67 (m, 1H), 3.87 (d, J = 10.9 Hz, 1H), 3.74 (d, J = 12.7 Hz, 1H), 3.68 (s, 3H), 3.62 (dd, J = 10.8, 4.3 Hz, 1H), 3.38 (dd, J = 12.6, 4.3 Hz, 1H), 2.03-2.10 (m, 1H), 1.96-2.02 (m, 1H), 1.48 (t, J = 3.5 Hz, 1H). | 428.1 |
| 1-((1R,5S,6s)-6- ((4-amino-5-(7- chlorobenzo[d] [1,3]dioxol-4- yl)-7-methyl- 7H-pyrrolo[2,3- d]pyrimidin-6- yl)ethynyl)-3- azabicyclo[3.1.0] hexan-3- yl)prop-2-en-1- one | 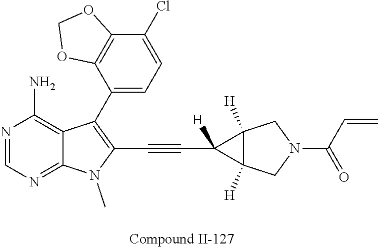 [SMILES: C=CC(=O)N1C[C@@H]2[C@@H](C#Cc3c(-c4ccc(Cl)c5c4OCO5)c4c(N)ncnc4n3C)[C@@H]2C1] | ¹H NMR (400 MHz, DMSO-d₆): δ = 8.14 (s, 1H), 7.03 (d, J = 8.5 Hz, 1H), 6.86 (d, J = 8.5 Hz, 1H), 6.49 (br dd, J = 16.6, 10.3 Hz, 1H), 6.14 (br s, 2H), 6.10 (br dd, J = 16.8, 2.2 Hz, 1H), 5.65 (br dd, J = 10.4, 2.1 Hz, 1H), 3.88 (br d, J = 10.7 Hz, 1H), 3.75 (brd, J = 12.5 Hz, 1H), 3.68 (s, 3H), 3.63 (br dd, J = 10.7, 4.2 Hz, 1H), 3.39 (br dd, J = 12.7, 4.2 Hz, 1H), 2.05-2.13 (m, 1H), 2.02 (br d, J = 3.4 Hz, 1H), 1.49 (br t, J = 3.2 Hz, 1H). | 462.1 |
| 1-((1R,5S,6s)-6- ((4-amino-5-(7- chloro-2,2- difluorobenzo[d] [1,3]dioxol-4- yl)-7-methyl- 7H-pyrrolo[2,3- d]pyrimidin-6- yl)ethynyl)-3- azabicyclo[3.1.0] hexan-3- yl)prop-2-en-1- one | 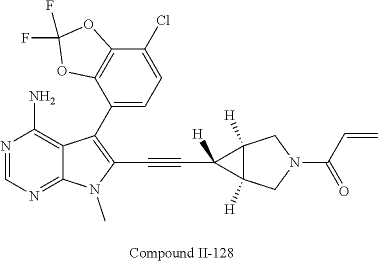 [SMILES: C=CC(=O)N1C[C@@H]2[C@@H](C#Cc3c(-c4ccc(Cl)c5c4OC(F)(F)O5)c4c(N)ncnc4n3C)[C@@H]2C1] | ¹H NMR (500 MHz, DMSO-d₆): δ = 8.18 (s, 1H), 7.42 (d, J = 8.8 Hz, 1H), 7.18 (d, J = 8.8 Hz, 1H), 6.49 (br dd, J = 16.7, 10.2 Hz, 2H), 6.09 (dd, J = 16.7, 1.9 Hz, 1H), 5.61-5.67 (m, 1H), 3.85 (d, J = 10.9 Hz, 1H), 3.67-3.76 (m, 4H), 3.63 (dd, J = 10.8, 4.3 Hz, 1H), 3.39 (dd, J = 12.6, 4.3 Hz, 1H), 1.98-2.10 (m, 2H), 1.46 (t, J = 3.2 Hz, 1H). | 498.1 |
| 1-((1R,5S,6s)-6- ((4-amino-5- (2,2- difluorobenzo[d] [1,3]dioxol-4- yl)-7-methyl- 7H-pyrrolo[2,3- d]pyrimidin-6- yl)ethynyl)-3- azabicyclo[3.1.0] hexan-3- yl)prop-2-en-1- one | 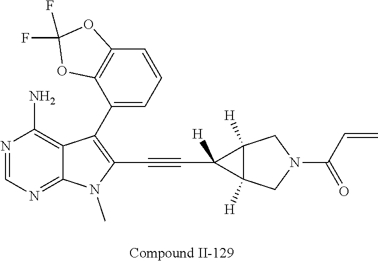 [SMILES: C=CC(=O)N1C[C@@H]2[C@@H](C#Cc3c(-c4cccc5c4OC(F)(F)O5)c4c(N)ncnc4n3C)[C@@H]2C1] | ¹H NMR (400 MHz, DMSO-d₆): δ = 8.19 (s, 1H), 7.41 (dd, J = 8.1, 1.0 Hz, 1H), 7.31 (t, J = 8.1 Hz, 1H), 7.17 (dd, J = 8.1, 1.0 Hz, 1H), 6.49 (dd, J = 16.8, 10.3 Hz, 1H), 6.35 (br s, 2H), 6.10 (dd, J = 16.7, 2.3 Hz, 1H), 5.60- 5.68 (m, 1H), 3.85 (d, J = 11.0 Hz, 1H), 3.67-3.77 (m, 4H), 3.63 (dd, J = 10.7, 4.2 Hz, 1H), 3.38 (dd, J = 12.6, 4.3 Hz, 1H), 1.93-2.09 (m, 2H), 1.45 (t, J = 3.4 Hz, 1H). | 464.1 |
| 1-((1R,5S,6s)-6- ((4-amino-5-(7- fluorobenzo[d] [1,3]dioxol-4- yl)-7-methyl- 7H-pyrrolo[2,3- d]pyrimidin-6- yl)ethynyl)-3- azabicyclo[3.1.0] hexan-3- yl)prop-2-en-1- one | 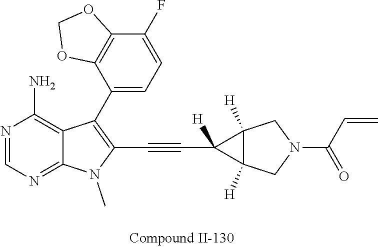 [SMILES: C=CC(=O)N1C[C@@H]2[C@@H](C#Cc3c(-c4ccc(F)c5c4OCO5)c4c(N)ncnc4n3C)[C@@H]2C1] | ¹H NMR (500 MHz, DMSO-d₆): δ = 8.14 (s, 1H), 6.91-7.01 (m, 1H), 6.86 (dd, J = 8.8, 4.9 Hz, 1H), 6.49 (dd, J = 16.9, 10.4 Hz, 1H), 6.13 (s, 2H), 6.10 (dd, J = 16.9, 2.3 Hz, 1H), 5.63-5.67 (m, 1H), 3.87 (d, J = 10.6 Hz, 1H), 3.75 (d, J = 12.4 Hz, 1H), 3.68 (s, 3H), 3.63 (dd, J = 10.6, 4.1 Hz, 1H), 3.39 (dd, J = 12.6, 4.3 Hz, 1H), 1.97- 2.10 (m, 2H), 1.48 (t, J = 3.4 Hz, 1H). | 446.2 |
| 1-((1R,5S,6s)-6- ((4-amino-5-(7- fluorobenzo[d] [1,3]dioxol-4- yl)-7-isopropyl- 7H-pyrrolo[2,3- d]pyrimidin-6- yl)ethynyl)-3- azabicyclo[3.1.0] hexan-3- yl)prop-2-en-1- one | 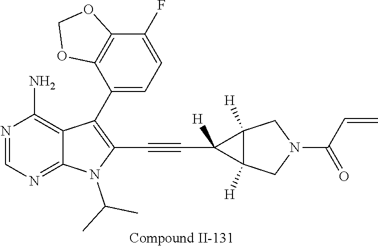 [SMILES: C=CC(=O)N1C[C@@H]2[C@@H](C#Cc3c(-c4ccc(F)c5c4OCO5)c4c(N)ncnc4n3C(C)C)[C@@H]2C1] | ¹H NMR (400 MHz, DMSO-d₆): δ = 8.12 (s, 1H), 6.91-6.99 (m, 1H), 6.81- 6.90 (m, 1H), 6.49 (dd, J = 16.7, 10.4 Hz, 1H), 6.06-6.17 (m, 3H), 5.60-5.69 (m, 1H), 5.04 (dquin, J = 13.7, 6.8 Hz, 1H), 3.88 (br d, J = 11.0 Hz, 1H), 3.75 (br d, J = 12.5 Hz, 1H), 3.62 (br dd, J = 10.7, 4.2 Hz, 1H), 3.39 (br dd, J = 12.7, 4.2 Hz, 1H), 1.92-2.08 (m, 2H), 1.59 (d, J = 7.1 Hz, 6H), 1.49 (t, J = 3.3 Hz, 1H). | 474.1 |
| 1-((1R,5S,6s)-6- ((4-amino-5- (benzo[d][1,3] dioxol-4-yl)-7- isopropyl-7H- pyrrolo[2,3- d]pyrimidin-6- yl)ethynyl)-3- azabicyclo[3.1.0] hexan-3- yl)prop-2-en-1- one | 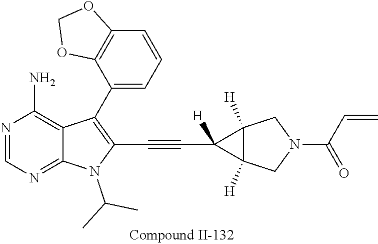 [SMILES: C=CC(=O)N1C[C@@H]2[C@@H](C#Cc3c(-c4cccc5c4OCO5)c4c(N)ncnc4n3C(C)C)[C@@H]2C1] | ¹H NMR (500 MHz, DMSO-d₆): δ = 8.13 (s, 1H), 6.93-7.01 (m, 2H), 6.86 (dd, J = 6.5, 2.6 Hz, 1H), 6.48 (dd, J = 16.6, 10.4 Hz, 1H), 6.09 (br dd, J = 16.7, 2.2 Hz, 1H), 6.03 (br d, J = 5.7 Hz, 2H), 5.61-5.68 (m, 1H), 5.00-5.10 (m, 1H), 3.87 (br d, J = 10.9 Hz, 1H), 3.74 (br d, J = 12.4 Hz, 1H), 3.62 (br dd, J = 10.8, 3.8 Hz, 1H), 3.38 (br dd, J = 12.7, 3.9 Hz, 1H), 1.93-2.05 (m, 2H), 1.59 (d, J = 6.7 Hz, 6H), 1.48 (t, J = 3.2 Hz, 1H). | 456.2 |
| 1-((1R,5S,6s)-6- ((4-amino-5-(7- fluorobenzo[d] [1,3]dioxol-4- yl)-7-isopropyl- 7H-pyrrolo[2,3- d]pyrimidin-6- yl)ethynyl)-3- azabicyclo[3.1.0] hexan-3- yl)propan-1-one | 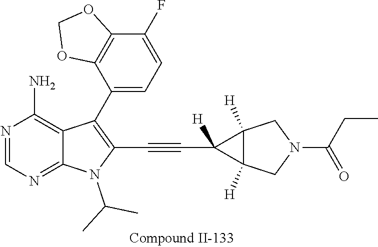 [SMILES: CCC(=O)N1C[C@@H]2[C@@H](C#Cc3c(-c4ccc(F)c5c4OCO5)c4c(N)ncnc4n3C(C)C)[C@@H]2C1] | ¹H NMR (500 MHz, DMSO-d₆): δ = 8.12 (s, 1H), 6.90-6.99 (m, 1H), 6.83- 6.88 (m, 1H), 6.13 (br d, J = 13.2 Hz, 2H), 5.04 (dt, J = 13.7, 6.9 Hz, 1H), 3.67 (br d, J = 12.2 Hz, 2H), 3.52 (br dd, J = 10.5, 3.8 Hz, 1H), 3.25-3.30 (m, 1H), 2.08-2.26 (m, 2H), 1.88-2.03 (m, 2H), 1.58 (d, J = 7.0 Hz, 6H), 1.43 (t, J = 3.1 Hz, 1H), 0.93 (t, J = 7.4 Hz, 3H). | 476.2 |
| 1-(4-(3-((4- amino-5-(7- fluorobenzo[d] [1,3]dioxol-4- yl)-7-isopropyl- 7H-pyrrolo[2,3- d]pyrimidin-6- yl)ethynyl) azetidin-1- yl)piperidin-1- yl)prop-2-en-1- one | 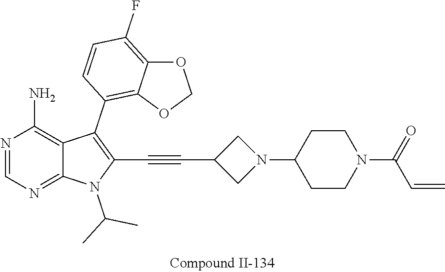 [SMILES: C=CC(=O)N1CCC(N2CC(C#Cc3c(-c4ccc(F)c5c4OCO5)c4c(N)ncnc4n3C(C)C)C2)CC1] | ¹H NMR (500 MHz, DMSO-d₆): δ 8.00-8.22 (m, 1H), 6.84-7.01 (m, 1H), 6.64-6.82 (m, 1H), 5.97-6.27 (m, 3H), 5.50-5.71 (m, 1H), 4.98-5.17 (m, 1H), 3.88-4.05 (m, 1H), 3.70-3.85 (m, 1H), 3.44-3.59 (m, 1H), 3.35-3.43 (m, 1H), 3.10-3.24 (m, 1H), 3.03 (s, 1H), 2.84- 2.96 (m, 1H), 2.15-2.34 (m, 1H), 1.47-1.71 (m, 6H). | 531 |
| 1-((1R,5S,6s)-6- ((4-amino-7- methyl-5-(7-((6- methylpyridin- 2- yl)oxy)benzo[d] [1,3]dioxol-4- yl)-7H- pyrrolo[2,3- d]pyrimidin-6- yl)ethynyl)-3- azabicyclo[3.1.0] hexan-3- yl)prop-2-en-1- one | 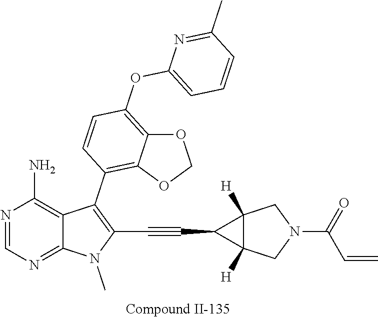 [SMILES: C=CC(=O)N1C[C@@H]2[C@@H](C#Cc3c(-c4ccc(Oc5cccc(C)n5)c5c4OCO5)c4c(N)ncnc4n3C)[C@@H]2C1] | ¹H NMR (500 MHz, DMSO-d₆): δ 7.50 (t, J =7.78 Hz, 1H), 6.89-6.98 (m, 1H), 6.79-6.84 (m, 1H), 6.73-6.79 (m, 1H), 6.55-6.61 (m, 1H), 6.43-6.53 (m, 1H), 5.97-6.18 (m, 4H), 5.56-5.70 (m, 1H), 3.82-3.93 (m, 1H), 3.67-3.78 (m, 2H), 3.58-3.66 (m, 1H), 3.53-3.56 (m, 3H), 2.14-2.26 (m, 4H), 1.89-2.04 (m, 2H), 1.33-1.44 (m, 1H). | 535 |

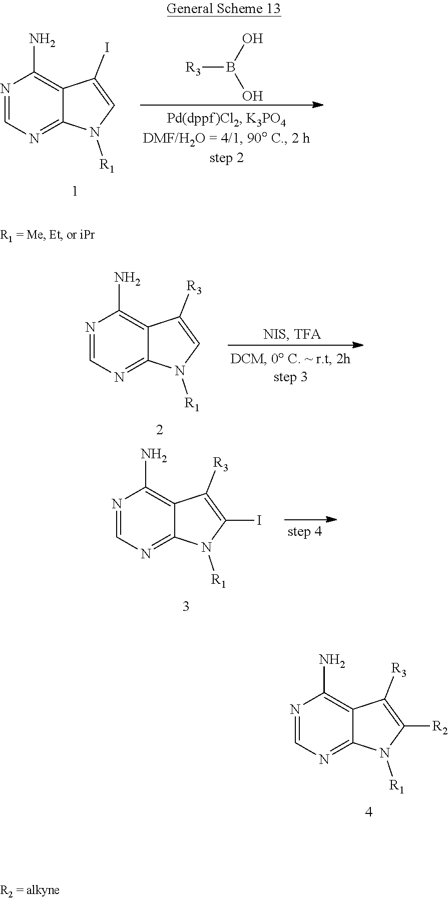

### Example 21

### 1-(4-(3-((4-amino-5-(4-chloro-3-methoxyphenyl)-7-methyl-7H-pyrrolo[2,3-d]pyrimidin-6-yl)ethynyl)azetidin-1-yl)piperidin-1-yl)prop-2-en-1-one

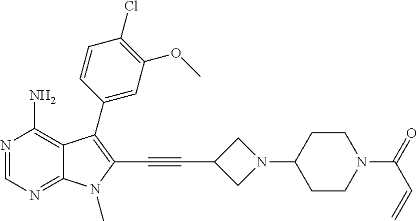 [SMILES: C=CC(=O)N1CCC(N2CC(C#Cc3c(-c4ccc(Cl)c(OC)c4)c4c(N)ncnc4n3C)C2)CC1]

### 5-(4-chloro-3-methoxyphenyl)-7-methyl-7H-pyrrolo[2,3-d]pyrimidin-4-amine

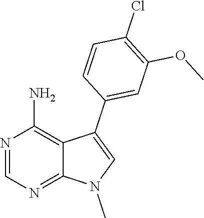 [SMILES: COc1cc(-c2cn(C)c3ncnc(N)c23)ccc1Cl]

### Step 1

A round bottomed flask was charged with 5-iodo-7-methyl-7H-pyrrolo[2,3-d]pyrimidin-4-amine (3 g, 10.9 mmol), Pd(dppf)Cl₂ (796 mg, 1.09 mmol), K₃PO₄ (6.91 g, 32.6 mmol) and a stirbar. DMF/H₂O=8/1 (45 mL) was added, and the solution was stirred at 90° C. under N₂ for 2 h. The reaction mixture was diluted with H₂O (500 mL), and the aqueous phase was extracted with dichloromethane (500 mL) three times. The combined organic layers were washed with brine, dried over sodium sulfate, filtered, and concentrated in vacuo. The resulting crude material was purified by silica gel chromatography (MeOH/DCM=1/30). Concentration in vacuo resulted in 5-(4-chloro-3-methoxyphenyl)-7-methyl-7H-pyrrolo[2,3-d]pyrimidin-4-amine (2.20 g, 70%).

### 5-(4-chloro-3-methoxyphenyl)-6-iodo-7-methyl-7H-pyrrolo[2,3-d]pyrimidin-4-amine

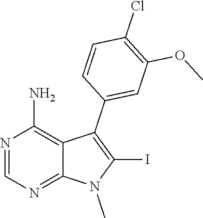 [SMILES: COc1cc(-c2c(I)n(C)c3ncnc(N)c23)ccc1Cl]

### Step 2

A round bottomed flask was charged with 5-(4-chloro-3-methoxyphenyl)-7-methyl-7H-pyrrolo[2,3-d]pyrimidin-4-amine (2.2 g, 7.61 mmol), TFA (2.59 g, 22.8 mmol), DCM (20 mL) and a stirbar. The mixture was cooled to 0° C. and NIS (1.71 g, 7.61 mmol) was added, and the solution was stirred for 2 h at room temperature. The pH of the solution was adjusted to 7-8 with saturated Na₂SO₃ solution. The solid was filtered and washed with H₂O, then washed with a small amount of DCM and resulted in 5-(4-chloro-3-methoxyphenyl)-6-iodo-7-methyl-7H-pyrrolo[2,3-d]pyrimidin-4-amine (2.50 g, 79%).

### tert-butyl 4-(3-((4-amino-5-(4-chloro-3-methoxyphenyl)-7-methyl-7H-pyrrolo[2,3-d]pyrimidin-6-yl)ethynyl)azetidin-1-yl)piperidine-1-carboxylate

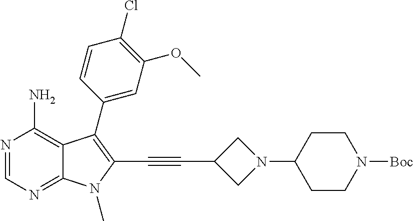 [SMILES: COc1cc(-c2c(C#CC3CN(C4CCN(C(=O)OC(C)(C)C)CC4)C3)n(C)c3ncnc(N)c23)ccc1Cl]

### Step 3

A round bottomed flask was charged with 5-(4-chloro-3-methoxyphenyl)-6-iodo-7-methyl-7H-pyrrolo[2,3-d]pyrimidin-4-amine (240 mg, 0.58 mmol), tert-butyl 4-(3-ethynylazetidin-1-yl)piperidine-1-carboxylate (183 mg, 0.70 mmol), Pd(PPh₃)₂Cl₂ (85 mg, 0.11 mmol), CuI (43.9 mg, 0.23 mmol), TEA (291 mg, 2.89 mmol) and a stirbar. Dimethylformamide (10 mL) was added, and the solution was stirred at 50° C. for 3 h under N₂. The resulting crude material was purified by silica gel chromatography (hexanes/ethyl acetate=10/1). Yielded tert-butyl 4-(3-{2-[4-amino-5-(4-chloro-3-methoxyphenyl)-7-methyl-7H-pyrrolo[2,3-d]pyrimidin-6-yl]ethynyl}azetidin-1-yl)piperidine-1-carboxylate (138.0 mg, 43.2%).

The remaining steps were carried out according to procedures described herein.

### Example 22

### 1-(4-(3-((4-amino-5-(4-chloro-3-methoxyphenyl)-7-methyl-7H-pyrrolo[2,3-d]pyrimidin-6-yl)ethynyl)-3-fluoroazetidine-1-yl)piperidin-1-yl)prop-2-en-1-one

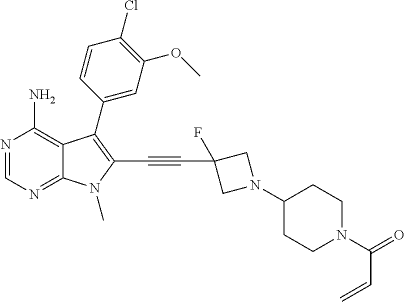 [SMILES: C=CC(=O)N1CCC(N2CC(F)(C#Cc3c(-c4ccc(Cl)c(OC)c4)c4c(N)ncnc4n3C)C2)CC1]

### tert-butyl 3-((4-amino-5-(4-chloro-3-methoxyphenyl)-7-methyl-7H-pyrrolo[2,3-d]pyrimidin-6-yl)ethynyl)-3-fluoroazetidine-1-carboxylate

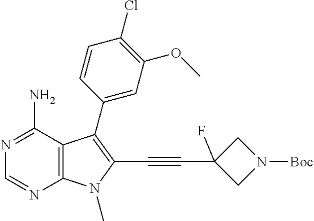 [SMILES: COc1cc(-c2c(C#CC3(F)CN(C(=O)OC(C)(C)C)C3)n(C)c3ncnc(N)c23)ccc1Cl]

### Step 1

To a solution of tert-butyl 3-{2-[4-amino-5-(4-chloro-3-methoxyphenyl)-7-methyl-7H-pyrrolo[2,3-d]pyrimidin-6-yl]ethynyl}-3-hydroxyazetidine-1-carboxylate (190 mg, 392 μmol) in absolute DCM (10 mL), cooled at −78° C., was added DAST (126 mg, 784 μmol) dropwise over 5 min. The reaction was stirred at −78° C. for 1 hour then allowed to warm to 0° C. for 30 min. The reaction mixture was poured onto sat. aq. NaHCO₃ (10 mL). The two layers were separated, and aq. layer was extracted with DCM (100 mL). The solvent was removed under reduced pressure. The crude product was purified by column chromatography on silica gel, elution gradient 0 to 2% MeOH in DCM. Pure fractions were evaporated to dryness to afford tert-butyl 3-{2-[4-amino-5-(4-chloro-3-methoxyphenyl)-7-methyl-7H-pyrrolo[2,3-d]pyrimidin-6-yl]ethynyl}-3-fluoroazetidine-1-carboxylate (110 mg, 58%).

The remaining steps were carried out according to procedures described herein.

### Example 23

### 1-((2S)-4-(3-((4-amino-5-(4-chloro-3-methoxyphenyl)-7-methyl-7H-pyrrolo[2,3-d]pyrimidin-6-yl)ethynyl)azetidin-1-yl)-2-(hydroxymethyl)piperidin-1-yl)prop-2-en-1-one

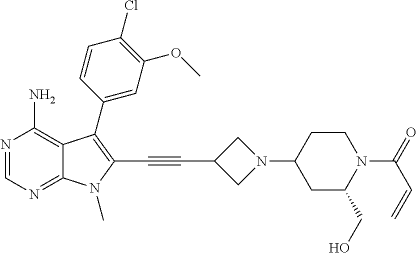 [SMILES: C=CC(=O)N1CCC(N2CC(C#Cc3c(-c4ccc(Cl)c(OC)c4)c4c(N)ncnc4n3C)C2)C[C@H]1CO]

### (2S)-tert-butyl 4-(3-((4-amino-5-(4-chloro-3-methoxyphenyl)-7-methyl-7H-pyrrolo[2,3-d]pyrimidin-6-yl)ethynyl)azetidin-1-yl)-2-(hydroxymethyl)piperidine-1-carboxylate

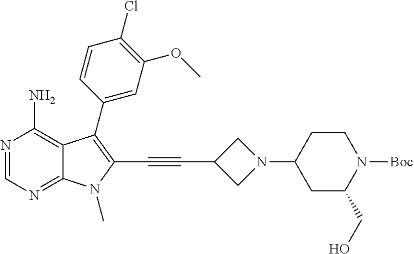 [SMILES: COc1cc(-c2c(C#CC3CN(C4CCN(C(=O)OC(C)(C)C)[C@H](CO)C4)C3)n(C)c3ncnc(N)c23)ccc1Cl]

### Step 1

A round bottomed flask was charged with 1-tert-butyl 2-methyl (2S)-4-(3-{2-[4-amino-5-(4-chloro-3-methoxyphenyl)-7-methyl-7H-pyrrolo[2,3-d]pyrimidin-6-yl]ethynyl}azetidin-1-yl)piperidine-1,2-dicarboxylate (210 mg, 344 μmol), dichloromethane (20 mL) and a stirbar. DIBAL-H (194 mg, 1.37 mmol) was added at −20° C., and the solution was stirred at −20° C. for 2 h. The reaction was then quenched by the addition of 10 mL of water. The mixture with DCM extraction. The resulting crude material was purified by silica gel chromatography (dichloromethane/methanol=20/1). Concentration in vacuo resulted in tert-butyl (2S)-4-(3-{2-[4-amino-5-(4-chloro-3-methoxyphenyl)-7-methyl-7H-pyrrolo[2,3-d]pyrimidin-6-yl]ethynyl}azetidin-1-yl)-2-(hydroxymethyl)piperidine-1-carboxylate (190 mg, 92%).

The remaining steps were carried out according to procedures described herein.

### Example 24

### (E)-1-((1R,5S,6s)-6-((4-amino-5-(4-chloro-3-methoxyphenyl)-7-methyl-7H-pyrrolo[2,3-d]pyrimidin-6-yl)ethynyl)-3-aza-bicyclo[3.1.0]hexan-3-yl)-4-(dimethylamino)but-2-en-1-one

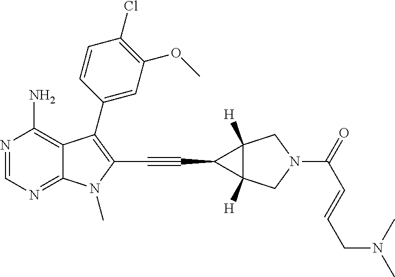 [SMILES: COc1cc(-c2c(C#C[C@@H]3[C@H]4CN(C(=O)/C=C/CN(C)C)C[C@@H]34)n(C)c3ncnc(N)c23)ccc1Cl]

### Step 1

A solution of (2E)-4-(dimethylamino)but-2-enoic acid (50.5 mg, 0.39 mmol) in DMF (5 mL) was treated with HATU (148.66 mg, 0.39 mmol) for 5 min at room temperature under nitrogen atmosphere followed by the addition of 6-[2-[(1R,5S,6S)-3-azabicyclo[3.1.0]hexan-6-yl]ethynyl]-5-(4-chloro-3-methoxyphenyl)-7-methyl-7H-pyrrolo[2,3-d]pyrimidin-4-amine (140 mg, 0.36 mmol) and DIEA (459.4 mg, 3.55 mmol) dropwise at room temperature. The resulting mixture was stirred for 2 h at room temperature under nitrogen atmosphere. The reaction was monitored by LCMS. The resulting mixture was extracted with EtOAc and water. The combined organic layers were dried over anhydrous Na₂SO₄. After filtration, the filtrate was concentrated under reduced pressure. The crude product was purified by Prep-HPLC to afford (2E)-1-[(1R,5S,6S)-6-[2-[4-amino-5-(4-chloro-3-methoxyphenyl)-7-methyl-7H-pyrrolo[2,3-d]pyrimidin-6-yl]ethynyl]-3-azabicyclo[3.1.0]hexan-3-yl]-4-(dimethylamino)but-2-en-1-one (38.4 mg, 61%).

The remaining steps were carried out according to procedures described herein.

### Example 25

### 1-((3S,4S)-4-(3-((4-amino-5-(4-chloro-3-methoxyphenyl)-7-methyl-7H-pyrrolo[2,3-d]pyrimidin-6-yl)ethynyl)azetidin-1-yl)-3-hydroxypiperidin-1-yl)prop-2-en-1-one

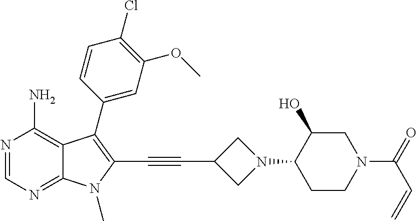 [SMILES: C=CC(=O)N1CC[C@H](N2CC(C#Cc3c(-c4ccc(Cl)c(OC)c4)c4c(N)ncnc4n3C)C2)[C@@H](O)C1]

### (3S,4S)-tert-butyl 4-(3-((4-amino-5-(4-chloro-3-methoxyphenyl)-7-methyl-7H-pyrrolo[2,3-d]pyrimidin-6-yl)ethynyl)azetidin-1-yl)-3-hydroxypiperidine-1-carboxylate

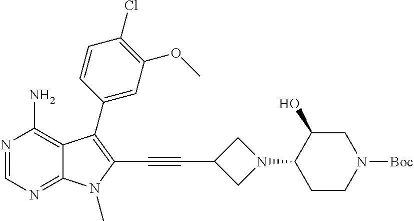 [SMILES: COc1cc(-c2c(C#CC3CN([C@H]4CCN(C(=O)OC(C)(C)C)C[C@@H]4O)C3)n(C)c3ncnc(N)c23)ccc1Cl]

### Step 1

A round bottomed flask was charged with 6-[2-(azetidin-3-yl)ethynyl]-5-(4-chloro-3-methoxyphenyl)-7-methyl-7H-pyrrolo[2,3-d]pyrimidin-4-amine (220 mg, 598 μmol), tert-butyl 7-oxa-3-azabicyclo[4.1.0]heptane-3-carboxylate (237 mg, 1.19 mmol) and a stirbar in EtOH (10 mL), and the solution was stirred at 100° C. for 16 hours. The solution was concentrated under vacuum. The resulting crude material was purified by silica gel chromatography (dichloromethane/methanol; ratio 60:1). Concentration in vacuo resulted in tert-butyl (3S,4S)-4-(3-{2-[4-amino-5-(4-chloro-3-methoxyphenyl)-7-methyl-7H-pyrrolo[2,3-d]pyrimidin-6-yl]ethynyl}azetidin-1-yl)-3-hydroxypiperidine-1-carboxylate (100 mg, 30%).

The remaining steps were carried out according to procedures described herein.

Compounds prepared by substantially the same methods, and their characterization data, are listed in Table 16, below.

TABLE 16
Selected Compounds

| | | | MS |
|---|---|---|---|
| | | | [M + |
| Compound | Structure | Proton NMR | 1] |
| 7-isopropyl-5-(4- phenoxyphenyl)-6- (piperidin-4- ylethynyl)-7H- pyrrolo[2,3- d]pyrimidin-4- amine | 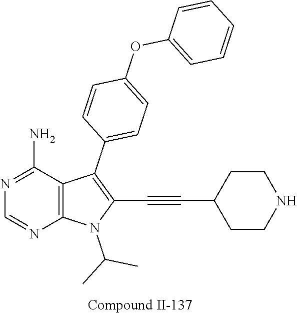 [SMILES: CC(C)n1c(C#CC2CCNCC2)c(-c2ccc(Oc3ccccc3)cc2)c2c(N)ncnc21] | ¹H NMR (300 MHz, DMSO-d₆) δ 8.16 (s, 1H), 7.52 − 7.45 (m, 2H), 7.43 − 7.39 (m, 2H), 7.20 − 6.97 (m, 5H), 6.14 (s, 1H), 5.13 (p, J = 6.9 Hz, 1H), 2.81 (tt, J = 12.8, 4.3 Hz, 3H), 2.60 − 2.50 (m, 1H), 2.28 (m, 1H), 1.72 (ddd, J = 12.9, 6.1, 3.4 Hz, 2H), 1.62 (d, J = 6.9 Hz, 6H), 1.56 − 1.40 (m, 2H). | 452.3 |
| 4-((4-amino-7- isopropyl-5-(4- phenoxyphenyl)- 7H-pyrrolo[2,3- d]pyrimidin-6- yl)ethynyl) cyclohexanecarboxylic acid | 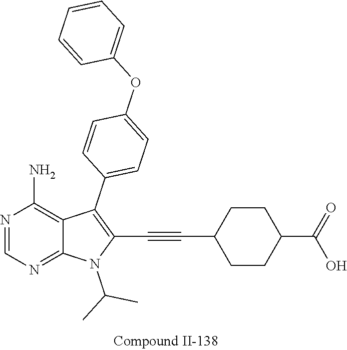 [SMILES: CC(C)n1c(C#CC2CCC(C(=O)O)CC2)c(-c2ccc(Oc3ccccc3)cc2)c2c(N)ncnc21] | ¹H NMR (300 MHz, DMSO-d₆) δ 8.15 (s, 1H), 7.51 (dd, J = 8.7, 3.0 Hz, 2H), 7.48 − 7.38 (m, 2H), 7.18 (t, J = 7.3 Hz, 1H), 7.12 (s, 1H), 7.12 (d, J = 8.4 Hz, 3H), 6.14 (s, 1H), 5.13 (p, J = 7.1 Hz, 1H), 2.16 (s, 1H), 1.93-1.86 (m, 3H), 1.70 − 1.63 (m, 8H), 1.39 (t, J = 9.4 Hz, 3H), 1.25 (s, 1H). | 495.4 |
| 1-(4-((4-amino-7- isopropyl-5-(4- phenoxyphenyl)- 7H-pyrrolo[2,3- d]pyrimidin-6- yl)ethynyl)piperidin- 1-yl)ethanone | 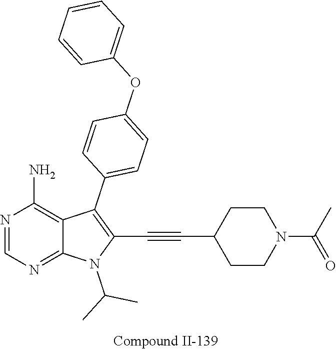 [SMILES: CC(=O)N1CCC(C#Cc2c(-c3ccc(Oc4ccccc4)cc3)c3c(N)ncnc3n2C(C)C)CC1] | ¹H NMR (400 MHz, Chloroform-d) δ 10.95 (s, 1H), 8.15 (s, 1H), 7.52 − 7.40 (m, 4H), 7.21 (t, J = 7.4 Hz, 1H), 7.11 − 7.08 (m, 4H), 5.73 (s, 1H), 5.18 (p, J = 6.9 Hz, 1H), 3.82 (dd, J = 12.1, 6.3 Hz, 1H), 3.57 − 3.53 (m, 1H), 3.42 − 3.28 (m, 2H), 2.93 (dq, J = 8.0, 4.0 Hz, 1H), 2.10 (s, 3H), 1.87 (dd, J = 6.8, 3.4 Hz, 4H), 1.72 (d, J = 6.9 Hz, 6H). | 494.2 |
| 7-isopropyl-6-((1- (methylsulfonyl) piperidin-4- yl)ethynyl)-5-(4- phenoxyphenyl)- 7H-pyrrolo[2,3- d]pyrimidin-4- amine | 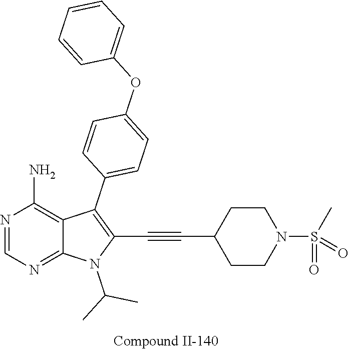 [SMILES: CC(C)n1c(C#CC2CCN(S(C)(=O)=O)CC2)c(-c2ccc(Oc3ccccc3)cc2)c2c(N)ncnc21] | ¹H NMR (300 MHz, DMSO-d₆) δ 8.17 (s, 1H), 7.52 − 7.41 (m, 4H), 7.21 − 7.10 (m, 5H), 6.14 (s, 1H), 5.14 − 5.09 (m, 1H), 3.32 − 3.19 (m, 2H), 3.07 − 2.90 (m, 3H), 2.82 (s, 3H), 1.91 (ddd, J = 10.2, 6.9, 3.6 Hz, 2H), 1.63 (d, J = 6.9 Hz, 8H). | 530.2 |
| 7-isopropyl-6-((1- methylpiperidin-4- yl)ethynyl)-5-(4- phenoxyphenyl)- 7H-pyrrolo[2,3- d]pyrimidin-4- amine | 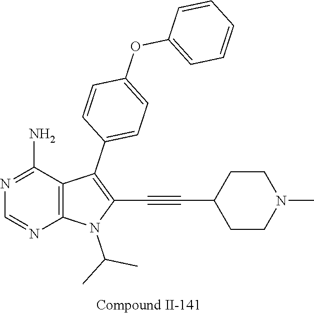 [SMILES: CC(C)n1c(C#CC2CCN(C)CC2)c(-c2ccc(Oc3ccccc3)cc2)c2c(N)ncnc21] | ¹H NMR (300 MHz, Chloroform-d) δ 8.31 (s, 1H), 7.53 (d, J = 8.6 Hz, 2H), 7.40 (t, J = 7.8 Hz, 2H), 7.18 (t, J = 7.4 Hz, 1H), 7.16 − 7.07 (m, 4H), 5.26 (p, J = 6.9 Hz, 1H), 5.03 (s, 2H), 2.63 (s, 3H), 2.29 (s, 5H), 1.94 (s, 2H), 1.80 (s, 2H), 1.72 (d, J = 7.0 Hz, 6H). | 466.3 |
| 7-isopropyl-6-((1- isopropylpiperidin- 4-yl)ethynyl)-5- (4- phenoxyphenyl)- 7H-pyrrolo[2,3- d]pyrimidin-4- amine | 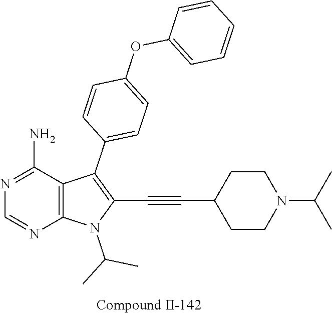 [SMILES: CC(C)N1CCC(C#Cc2c(-c3ccc(Oc4ccccc4)cc3)c3c(N)ncnc3n2C(C)C)CC1] | ¹H NMR (300 MHz, Chloroform-d) δ 8.34 (s, 1H), 7.63 − 7.31 (m, 4H), 7.22 − 7.09 (m, 5H), 5.24 (s, 1H), 5.05 (s, 2H), 2.80 (s, 4H), 1.85 (d, J = 16.6 Hz, 2H), 1.72 (d, J = 6.9 Hz, 6H), 1.61 (s, 4H), 1.16 (s, 6H). | 494.4 |
| 2-(4-((4-amino-7- isopropyl-5-(4- phenoxyphenyl)- 7H-pyrrolo[2,3- d]pyrimidin-6- yl)ethynyl)piperidin- 1-yl)-2- phenylethanol | 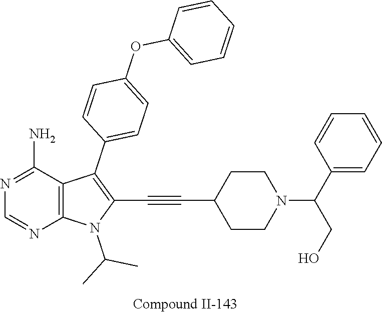 [SMILES: CC(C)n1c(C#CC2CCN(C(CO)c3ccccc3)CC2)c(-c2ccc(Oc3ccccc3)cc2)c2c(N)ncnc21] | ¹H NMR (400 MHz, DMSO-d₆) δ 8.15 (s, 1H), 7.47 − 7.40 (m, 4H), 7.31 − 7.16 (m, 6H), 7.12 − 7.08 (m, 4H), 6.12 (s, 1H), 5.08 (hept, J = 6.6 Hz, 1H), 4.40 (dd, J = 6.0, 4.6 Hz, 1H), 3.79 (ddd, J = 11.1, 6.5, 4.7 Hz, 1H), 3.64 (dt, J = 11.3, 6.0 Hz, 1H), 3.42 (t, J = 6.2 Hz, 1H), 2.65 (s, 3H), 2.25 (t, J = 9.9 Hz, 2H), 1.81 − 1.74 (m, 2H), 1.57 (d, J = 7.0 Hz, 8H). | 572.2 |
| 7-isopropyl-5-(4- phenoxyphenyl)-6- ((1-(1- phenylethyl)piperidin- 4-yl)ethynyl)- 7H-pyrrolo[2,3- d]pyrimidin-4- amine | 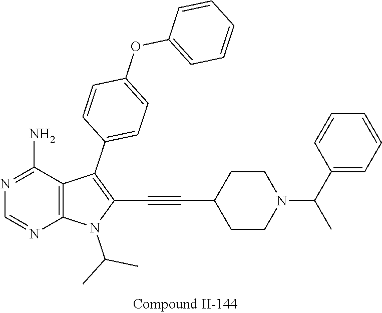 [SMILES: CC(c1ccccc1)N1CCC(C#Cc2c(-c3ccc(Oc4ccccc4)cc3)c3c(N)ncnc3n2C(C)C)CC1] | ¹H NMR (300 MHz, DMSO-d₆) δ 8.14 (s, 1H), 7.49 − 7.42 (m, 2H), 7.40 − 7.33 (m, 2H), 7.30 − 7.07 (m, 10H), 6.12 (s, 1H), 5.08 (p, J = 6.8 Hz, 1H), 3.40 (q, J = 6.5 Hz, 1H), 2.67 (s, 2H), 2.17 (d, J = 9.8 Hz, 2H), 1.76 (s, 2H), 1.58 (d, J = 6.9 Hz, 8H), 1.38 (d, J = 2.5 Hz, 1H), 1.25 (d, J = 6.7 Hz, 3H). | 556.3 |
| 6-((1- benzylpiperidin-4- yl)ethynyl)-7- isopropyl-5-(4- phenoxyphenyl)- 7H-pyrrolo[2,3- d]pyrimidin-4- amine | 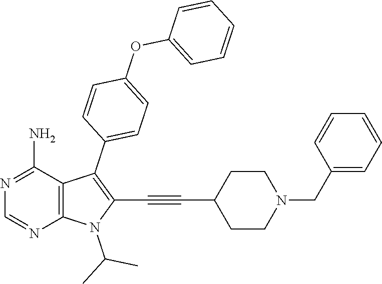 [SMILES: CC(C)n1c(C#CC2CCN(Cc3ccccc3)CC2)c(-c2ccc(Oc3ccccc3)cc2)c2c(N)ncnc21] | ¹H NMR (300 MHz, Chloroform-d) δ 8.31 (s, 1H), 7.56 − 7.51 (m, 2H), 7.41 − 7.32 (m, 3H), 7.34 (s, 3H), 7.38 − 7.26 (m, 1H), 7.20 − 7.11 (m, 1H), 7.17 − 7.08 (m, 4H), 5.23 (h, J = 6.8 Hz, 1H), 5.06 (s, 2H), 3.52 (s, 2H), 2.69 (s, 3H), 2.27 (s, 2H), 1.93 (s, 2H), 1.78 (td, J = 8.8, 3.9 Hz, 2H), 1.71 (d, J = 7.0 Hz, 6H). | 542.4 |
| | Compound II-145 | | |
| 2-(4-((4-amino-7- isopropyl-5-(4- phenoxyphenyl)- 7H-pyrrolo[2,3- d]pyrimidin-6- yl)ethynyl)piperidin- 1-yl)propan-1-ol | 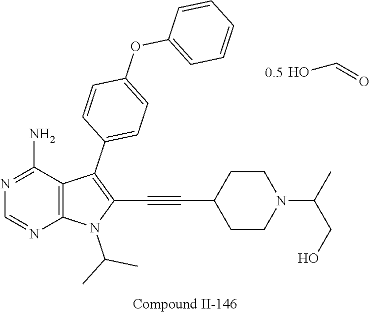 [SMILES: CC(CO)N1CCC(C#Cc2c(-c3ccc(Oc4ccccc4)cc3)c3c(N)ncnc3n2C(C)C)CC1 ; O=CO] | ¹H NMR (400 MHz, DMSO-d₆) δ 8.24 (s, 0.5H), 8.16 (s, 1H), 7.51 − 7.49 (m, 2H), 7.45 − 7.41 (m, 2H), 7.18 (td, J = 7.3, 1.1 Hz, 1H), 7.13 − 7.09 (m, 4H), 6.12 (s, 1H), 5.13 (p, J = 6.9 Hz, 1H), 3.42 (dd, J = 10.6, 6.3 Hz, 1H), 3.24 (dd, J = 10.6, 6.5 Hz, 1H), 2.69 (s, 2H), 2.67 − 2.53 (m, 2H), 2.45 − 2.34 (m, 2H), 1.79 (s, 2H), 1.62 (d, J = 6.9 Hz, 6H), 1.59 − 1.56 (m, 2H), 0.88 (d, J = 6.6 Hz, 3H). | 510.3 |
| 2-(4-((4-amino-7- isopropyl-5-(4- phenoxyphenyl)- 7H-pyrrolo[2,3- d]pyrimidin-6- yl)ethynyl)piperidin- 1-yl)ethanol | 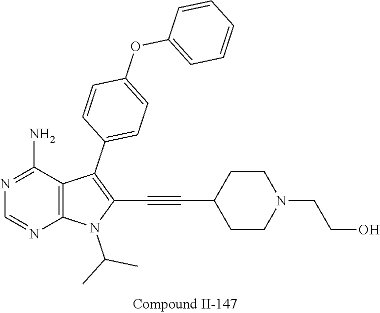 [SMILES: CC(C)n1c(C#CC2CCN(CCO)CC2)c(-c2ccc(Oc3ccccc3)cc2)c2c(N)ncnc21] | ¹H NMR (300 MHz, DMSO-d₆) δ 8.17 (s, 1H), 7.53 − 7.41 (m, 4H), 7.21 − 7.09 (m, 5H), 6.15 (s, 2H), 5.14 (p, J = 6.9 Hz, 1H), 4.35 (d, J = 5.4 Hz, 1H), 3.47 (d, J = 5.6 Hz, 2H), 2.72 (s, 1H), 2.58 (s, 2H), 2.35 (t, J = 6.3 Hz, 2H), 2.24 (s, 2H), 1.81 (d, J = 12.8 Hz, 2H), 1.63 (d, J = 6.9 Hz, 8H). | 496.3 |
| methyl 2-(4-((4- amino-7- isopropyl-5-(4- phenoxyphenyl)- 7H-pyrrolo[2,3- d]pyrimidin-6- yl)ethynyl)piperidin- 1-yl)propanoate | 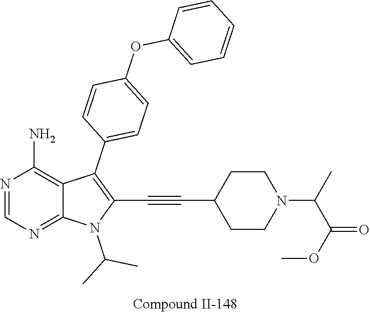 [SMILES: COC(=O)C(C)N1CCC(C#Cc2c(-c3ccc(Oc4ccccc4)cc3)c3c(N)ncnc3n2C(C)C)CC1] | ¹H NMR (400 MHz, DMSO-d₆) δ 8.16 (s, 1H), 7.50 − 7.48 (m, 2H), 7.44 − 7.40 (m, 2H), 7.20 − 7.16 (m, 1H), 7.12 − 7.09 (m, 4H), 6.12 (s, 2H), 5.13 (p, J = 6.9 Hz, 1H), 3.59 (s, 4H), 2.71 (dt, J = 7.3, 4.0 Hz, 3H), 2.42 (t, J = 10.4 Hz, 1H), 2.35 (t, J = 10.4 Hz, 1H), 1.80 (t, J = 10.4 Hz, 2H), 1.62 (d, J = 6.9 Hz, 6H), 1.55 (s, 2H), 1.16 (d, J = 7.0 Hz, 3H). | 538.4 |
| 2-(4-((4-amino-7- isopropyl-5-(4- phenoxyphenyl)- 7H-pyrrolo[2,3- d]pyrimidin-6- yl)ethynyl)piperidin- 1-yl)propanoic acid | 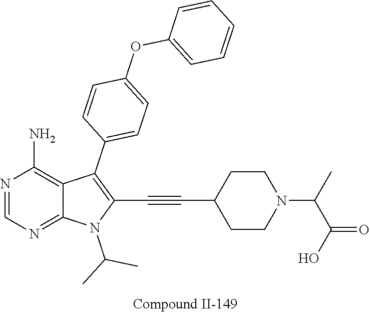 [SMILES: CC(C(=O)O)N1CCC(C#Cc2c(-c3ccc(Oc4ccccc4)cc3)c3c(N)ncnc3n2C(C)C)CC1] | ¹H NMR (400 MHz, DMSO-d₆) δ 8.16 (s, 1H), 7.52 − 7.48 (m, 2H), 7.46 − 7.42 (m, 2H), 7.23 − 7.14 (m, 1H), 7.14 − 7.07 (m, 4H), 6.14 (s, 1H), 5.13 (p, J = 6.9 Hz, 1H), 3.21 (p, J = 6.7 Hz, 1H), 2.81 (d, J = 13.3 Hz, 3H), 2.61 (dd, J = 21.2, 10.0 Hz, 2H), 1.92 − 1.84 (m, 2H), 1.62 (d, J = 6.9 Hz, 8H), 1.17 (d, J = 7.1 Hz, 3H). | 524.3 |
| 2-(4-((4-amino-7- isopropyl-5-(4- phenoxyphenyl)- 7H-pyrrolo[2,3- d]pyrimidin-6- yl)ethynyl)piperidin- 1- yl)propanamide | 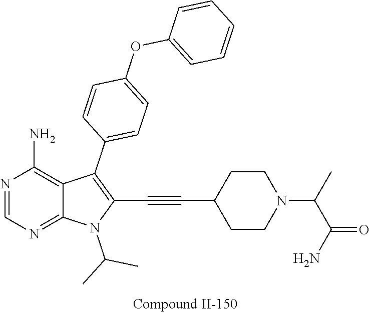 [SMILES: CC(C(N)=O)N1CCC(C#Cc2c(-c3ccc(Oc4ccccc4)cc3)c3c(N)ncnc3n2C(C)C)CC1] | ¹H NMR (400 MHz, DMSO-d₆) δ 8.15 (s, 1H), 7.49 (d, J = 8.6 Hz, 2H), 7.43 (dd, J = 8.6, 7.4 Hz, 2H), 7.18 (t, J = 7.2 Hz, 2H), 7.14 − 7.07 (m, 3H), 7.11 (s, 1H), 6.96 (s, 1H), 5.12 (q, J = 7.0 Hz, 1H), 2.96 (q, J = 6.9 Hz, 1H), 2.72 (s, 1H), 2.61 (s, 2H), 2.28 (s, 2H), 1.82 (s, 2H), 1.62 (d, J = 6.9 Hz, 8H), 1.03 (d, J = 6.9 Hz, 3H). | 523.3 |
| 6-((4- aminocyclohexyl) ethynyl)-7- isopropyl-5-(4- phenoxyphenyl)- 7H-pyrrolo[2,3- d]pyrimidin-4- amine | 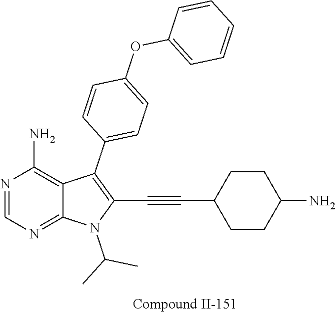 [SMILES: CC(C)n1c(C#CC2CCC(N)CC2)c(-c2ccc(Oc3ccccc3)cc2)c2c(N)ncnc21] | ¹H NMR (400 MHz, DMSO-d₆) δ 8.42 (s, 1H), 8.15 (s, 1H), 7.53 − 7.38 (m, 4H), 7.20 − 7.13 (m, 1H), 7.18 − 7.09 (m, 4H), 6.13 (s, 1H), 5.11 (p, J = 6.9 Hz, 1H), 2.84 (d, J = 11.4 Hz, 1H), 2.00 − 1.91 (m, 2H), 1.91 − 1.82 (m, 2H), 1.60 (d, J = 6.9 Hz, 6H), 1.48 − 1.34 (m, 2H), 1.33 − 1.19 (m, 2H). | 466.2 |
| 6-(azetidin-3- ylethynyl)-7- isopropyl-5-(4- phenoxyphenyl)- 7H-pyrrolo[2,3- d]pyrimidin-4- amine | 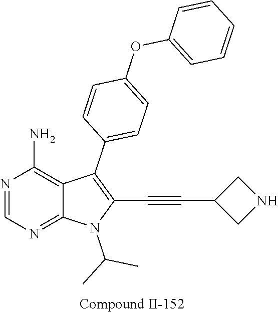 [SMILES: CC(C)n1c(C#CC2CNC2)c(-c2ccc(Oc3ccccc3)cc2)c2c(N)ncnc21] | ¹H NMR (400 MHz, DMSO-d₆) δ 8.16 (s, 1H), 7.60 − 7.41 (m, 4H), 7.19 − 7.10 (m, 5H), 6.14 (s, 2H), 5.10 (p, J = 6.7, 6.0 Hz, 1H), 1.62 (d, J = 6.8 Hz, 6H). | 424.1 |
| 6-((1-(azetidin-3- yl)piperidin-4- yl)ethynyl)-7- isopropyl-5-(4- phenoxyphenyl)- 7H-pyrrolo[2,3- d]pyrimidin-4- amine | 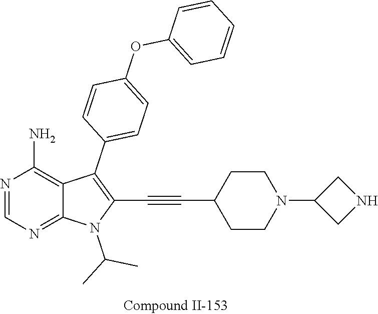 [SMILES: CC(C)n1c(C#CC2CCN(C3CNC3)CC2)c(-c2ccc(Oc3ccccc3)cc2)c2c(N)ncnc21] | ¹H NMR (400 MHz, DMSO-d₆) δ 8.16 (s, 1H), 7.53 − 7.47 (m, 2H), 7.44 (dd, J = 8.6, 7.3 Hz, 2H), 7.23 − 7.07 (m, 5H), 5.11 (p, J = 6.9 Hz, 1H), 3.28 (s, 4H), 2.98 (s, 1H), 2.74 (s, 1H), 2.37 (s, 2H), 2.03 (s, 2H), 1.79 (s, 2H), 1.62 (d, J = 6.9 Hz, 6H), 1.57 (s, 2H). | 507.2 |
| methyl 2-(4- amino-5-(4- phenoxyphenyl)-6- (piperidin-4- ylethynyl)-7H- pyrrolo[2,3- d]pyrimidin-7- yl)propanoate | 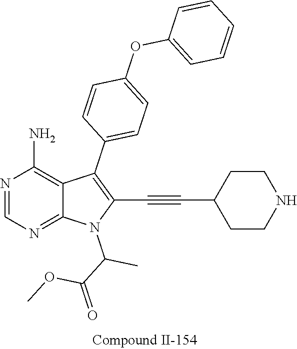 [SMILES: COC(=O)C(C)n1c(C#CC2CCNCC2)c(-c2ccc(Oc3ccccc3)cc2)c2c(N)ncnc21] | ¹H NMR (300 MHz, DMSO-d₆) δ 8.16 (s, 1H), 7.54 − 7.50 (m, 2H), 7.45 − 7.40 (m, 2H), 7.20 − 7.10 (m, 5H), 6.28 (s, 1H), 5.69 (q, J = 7.0 Hz, 1H), 3.65 (s, 3H), 2.86 − 2.71 (m, 3H), 2.55 (s, 2H), 1.70-1.68 (m, 5H), 1.48 − 1.41 (m, 2H). | 496.2 |
| 5-(4- phenoxyphenyl)-6- (piperidin-4- ylethynyl)-7- (tetrahydrofuran- 3-yl)-7H- pyrrolo[2,3- d]pyrimidin-4- amine | 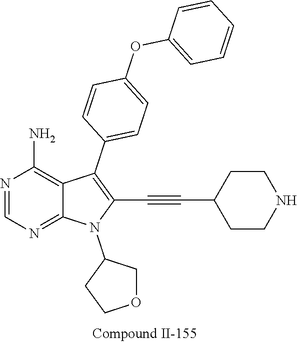 [SMILES: Nc1ncnc2c1c(-c1ccc(Oc3ccccc3)cc1)c(C#CC1CCNCC1)n2C1CCOC1] | ¹H NMR (400 MHz, DMSO-d₆) δ 8.19 (s, 1H), 7.59 − 7.38 (m, 4H), 7.24 − 7.04 (m, 5H), 5.53 (s, 1H), 4.25 − 3.86 (m, 4H), 2.83 (s, 3H), 2.65 (s, 2H), 2.33 (s, 2H), 1.75 (s, 2H), 1.49 (s, 2H), 1.15 (s, 1H). | 480.2 |
| 1-(4-((4-amino-5- (4- phenoxyphenyl)-7- (tetrahydrofuran- 3-yl)-7H- pyrrolo[2,3- d]pyrimidin-6- yl)ethynyl)piperidin- 1-yl)propan-1- one | 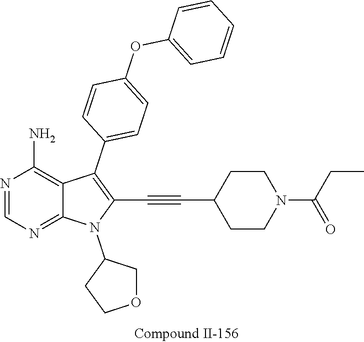 [SMILES: CCC(=O)N1CCC(C#Cc2c(-c3ccc(Oc4ccccc4)cc3)c3c(N)ncnc3n2C2CCOC2)CC1] | ¹H NMR (300 MHz, DMSO-d₆) δ 8.19 (s, 1H), 7.54 − 7.40 (m, 4H), 7.21 − 7.08 (m, 5H), 5.60 − 5.46 (m, 1H), 4.20 (q, J = 8.3 Hz, 1H), 4.07 (dd, J = 7.9, 2.9 Hz, 2H), 3.93 (q, J = 7.4 Hz, 1H), 3.54 (d, J = 34.7 Hz, 2H), 3.30-3.21 (m, 2H), 2.99 (dd, J = 7.7, 4.0 Hz, 1H), 2.63 (dd, J = 12.7, 6.7 Hz, 1H), 2.29 (q, J = 7.4 Hz, 3H), 1.77 (s, 2H), 1.51 (s, 2H), 0.97 (t, J = 7.4 Hz, 3H). | 536.3 |
| 7-isopropyl-5-(4- phenoxyphenyl)-6- (pyrrolidin-3- ylethynyl)-7H- pyrrolo[2,3- d]pyrimidin-4- amine | 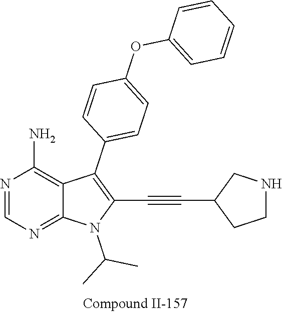 [SMILES: CC(C)n1c(C#CC2CCNC2)c(-c2ccc(Oc3ccccc3)cc2)c2c(N)ncnc21] | ¹H NMR (300 MHz, DMSO-d₆) δ 8.15 (s, 1H), 7.51 − 7.40 (m, 4H), 7.20 − 7.09 (m, 5H), 6.13 (s, 2H), 5.10 (p, J = 6.9 Hz, 1H), 3.17 − 3.06 (m, 1H), 3.02 − 2.92 (m, 1H), 2.78 (t, J = 6.9 Hz, 2H), 2.64 (dd, J = 10.4, 6.3 Hz, 1H), 2.02 (dt, J = 19.9, 7.4 Hz, 1H), 1.73-1.58 (m,7H). | 438.2 |
| 1-(3-(4-((4-amino- 7-isopropyl-5-(4- phenoxyphenyl)- 7H-pyrrolo[2,3- d]pyrimidin-6- yl)ethynyl)piperidin- 1-yl)azetidin-1- yl)propan-1-one | 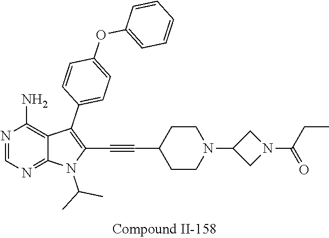 [SMILES: CCC(=O)N1CC(N2CCC(C#Cc3c(-c4ccc(Oc5ccccc5)cc4)c4c(N)ncnc4n3C(C)C)CC2)C1] | ¹H NMR (400 MHz, DMSO-d₆) δ 8.15 (s, 1H), 7.51 − 7.47 (m, 2H), 7.47 − 7.39 (m, 2H), 7.22 − 7.15 (m, 1H), 7.15 − 7.08 (m, 4H), 5.13 (p, J = 6.9 Hz, 1H), 4.05 (t, J = 7.9 Hz, 1H), 3.87 (dd, J = 8.6, 5.1 Hz, 1H), 3.80 (dd, J = 9.7, 7.2 Hz, 1H), 3.60 (dd, J = 9.8, 5.2 Hz, 1H), 3.02 (ddd, J = 12.4, 7.1, 5.2 Hz, 1H), 2.78 (s, 1H), 2.44 (s, 2H), 2.12 (s, 2H), 2.04 (q, J = 7.5 Hz, 2H), 1.83 (d, J = 13.3 Hz, 2H), 1.62 (d, J = 6.9 Hz, 8H), 0.95 (t, J = 7.5 Hz, 3H). | 563.3 |
| 7-methyl-5-(4- phenoxyphenyl)-6- (piperidin-4- ylethynyl)-7H- pyrrolo[2,3- d]pyrimidin-4- amine | 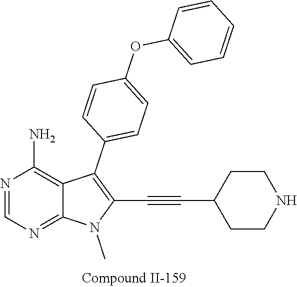 [SMILES: Cn1c(C#CC2CCNCC2)c(-c2ccc(Oc3ccccc3)cc2)c2c(N)ncnc21] | ¹H NMR (300 MHz, DMSO-d₆) δ 8.18 (s, 1H), 7.54 − 7.48 (m, 2H), 7.48 − 7.36 (m, 2H), 7.23 − 7.10 (m, 5H), 6.20 (s, 1H), 3.72 (s, 3H), 2.83 (dddd, J = 13.9, 10.5, 7.3, 4.0 Hz, 3H), 2.56 (dd, J = 8.7, 3.2 Hz, 2H), 1.74 (ddt, J = 13.1, 6.7, 3.6 Hz, 2H), 1.48 (dtd, J = 12.3, 8.6, 3.5 Hz, 2H). | 424.2 |
| 1-(4-((4-amino-5- (4-bromo-3- methoxyphenyl)- 7-isopropyl-7H- pyrrolo[2,3- d]pyrimidin-6- yl)ethynyl)piperidin- 1-yl)prop-2-en- 1-one | 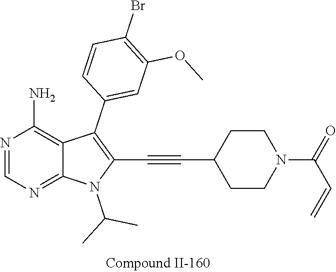 [SMILES: C=CC(=O)N1CCC(C#Cc2c(-c3ccc(Br)c(OC)c3)c3c(N)ncnc3n2C(C)C)CC1] | ¹H NMR (400 MHz, DMSO-d6): δ 8.04-8.19 (m, 2H), 7.59-7.72 (m, 2H), 7.11-7.21 (m, 2H), 6.90-7.07 (m, 2H), 6.63-6.86 (m, 3H), 5.95- 6.14 (m, 3H), 5.65 (dd, J =2.32, 10.38 Hz, 2H), 5.04-5.21 (m, 2H), 3.84-3.89 (m, 3H), 3.77-3.83 (m, 1H), 3.67-3.75 (m, 1H), 2.89-3.08 (m, 3H), 1.73-1.99 (m, 4H), 1.60 (d, J = 7.08 Hz, 6H). | 523 |
| 1-(4-(3-((4-amino- 5-(4-bromo-3- methoxyphenyl)- 7-isopropyl-7H- pyrrolo[2,3- d]pyrimidin-6- yl)ethynyl)azetidin- 1-yl)piperidin-1- yl)prop-2-en-1-one | 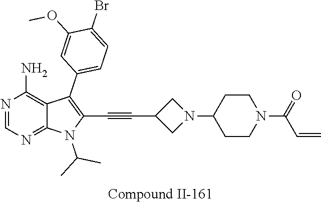 [SMILES: C=CC(=O)N1CCC(N2CC(C#Cc3c(-c4ccc(Br)c(OC)c4)c4c(N)ncnc4n3C(C)C)C2)CC1] | ¹H NMR (500 MHz, DMSO-d6): δ 8.15-8.15 (m, 1H), 8.12-8.21 (m, 1H), 8.07-8.26 (m, 1H), 8.21 (br s, 1H), 7.60-7.74 (m, 1H), 7.11-7.20 (m, 1H), 6.93-7.04 (m, 1H), 6.64- 6.85 (m, 1H), 5.97-6.13 (m, 1H), 5.57-5.69 (m, 1H), 5.01-5.19 (m, 1H), 3.92-4.01 (m, 1H), 3.86-3.92 (m, 2H), 3.74-3.84 (m, 1H), 3.48- 3.58 (m, 1H), 3.37-3.46 (m, 1H), 3.11-3.23 (m, 1H), 2.90-3.03 (m, 2H), 2.18-2.26 (m, 1H), 1.51-1.66 (m, 6H). | 577 |
| N-(3-(4-amino-7- methyl-5-(4- phenoxyphenyl)- 7H-pyrrolo[2,3- d]pyrimidin-6- yl)prop-2-yn-1- yl)acrylamide | 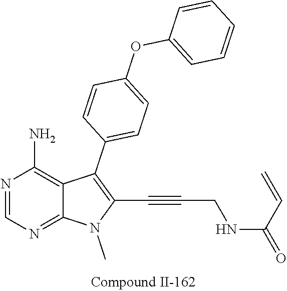 [SMILES: C=CC(=O)NCC#Cc1c(-c2ccc(Oc3ccccc3)cc2)c2c(N)ncnc2n1C] | ¹H NMR (400 MHz, DMSO-d₆) δ 8.64 (s, 1H), 8.19 (s, 1H), 7.62-7.42 (m, J = 26.3, 7.9 Hz, 4H), 7.21-7.08 (m, J = 7.3 Hz, 5H), 6.48-6.10 (m, J = 17.3, 10.0 Hz, 3H), 5.63-5.61 (d, J = 10.0 Hz, 1H), 4.25-4.24 (d, J = 5.5 Hz, 2H), 3.73 (s, 3H). MS: 424.15 | 424.2 |
| N-(3-(4-amino-7- methyl-5-(4- phenoxyphenyl)- 7H-pyrrolo[2,3- d]pyrimidin-6- yl)prop-2-yn-1- yl)-N- methylacrylamide | 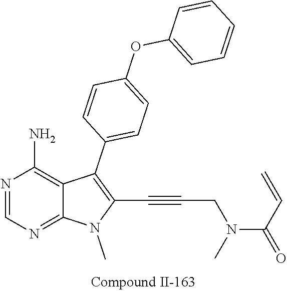 [SMILES: C=CC(=O)N(C)CC#Cc1c(-c2ccc(Oc3ccccc3)cc2)c2c(N)ncnc2n1C] | ¹H NMR (400 MHz, DMSO-d₆) δ 8.20 (s, 1H), 7.51 − 7.42 (m, 4H), 7.21-7.14 (t, J = 7.3 Hz, 1H), 7.12- 7.09 (dd, J = 11.7, 8.1 Hz, 4H), 6.83 − 6.72 (m, 1H), 6.24-6.23 (dd, J = 16.9, 8.6 Hz, 1H), 6.17-6.11 (m, 8.6 Hz, 1H) 5.73 − 5.64(m, 1H), 4.59- 4.50 (d, 2H), 3.73 (s, 3H), 3.08 − 2.95 (d, 3H). MS: 438.10 | 438.1 |
| 1-((3R,4S)-4-(3- ((4-amino-7- methyl-5-(4- phenoxyphenyl)- 7H-pyrrolo[2,3- d]pyrimidin-6- yl)ethynyl)azetidin- 1-yl)-3- hydroxypiperidin- 1-yl)prop-2-en-1- one | 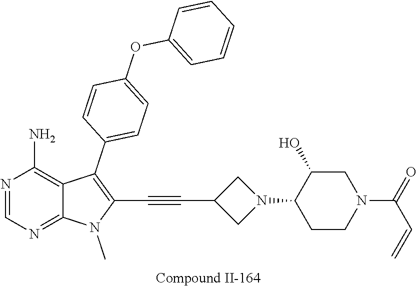 [SMILES: C=CC(=O)N1CC[C@H](N2CC(C#Cc3c(-c4ccc(Oc5ccccc5)cc4)c4c(N)ncnc4n3C)C2)[C@H](O)C1] | ¹H NMR (400 MHz, DMSO-d₆) δ 8.19 (s, 1H), 7.53 − 7.51 (m, 2H), 7.45 − 7.41 (m, 2H), 7.20 − 7.12 (m, 5H), 6.74 (ddd, J = 18.6, 16.7, 10.5 Hz, 1H), 6.22 (s, 1H), 6.05 (dd, J = 16.8, 2.5 Hz, 1H), 5.63 (ddd, J = 10.6, 5.1, 2.4 Hz, 1H), 4.46 (dd, J = 50.9, 3.4 Hz, 1H), 4.01 (d, J = 12.6 Hz, 1H), 3.79 (dd, J = 13.9, 5.0 Hz, 1H), 3.70 − 3.52 (m, 4H), 3.44 (p, J = 7.6 Hz, 1H), 3.19 − 2.84 (m, 3H), 2.40 − 2.23 (m, 1H), 1.36 (q, J = 7.3, 5.7 Hz, 2H). | 549.30 |
| 1-((3S,4R)-4-(3- ((4-amino-7- methyl-5-(4- phenoxyphenyl)- 7H-pyrrolo[2,3- d]pyrimidin-6- yl)ethynyl)azetidin- 1-yl)-3- hydroxypiperidin- 1-yl)prop-2-en-1- one | 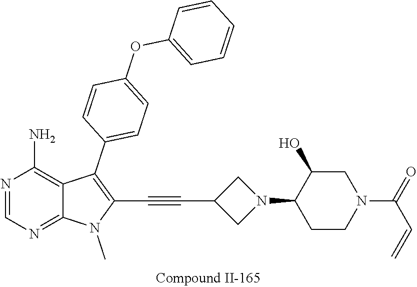 [SMILES: C=CC(=O)N1CC[C@@H](N2CC(C#Cc3c(-c4ccc(Oc5ccccc5)cc4)c4c(N)ncnc4n3C)C2)[C@@H](O)C1] | ¹H NMR (400 MHz, DMSO-d₆) δ 8.19 (s, 1H), 7.563- 7.51 (m, 2H), 7.47 − 7.38 (m, 2H), 7.24 − 7.07 (m, 5H), 6.82 − 6.67 (m, 1H), 6.21 (s, 1H), 6.05 (dd, J = 16.8, 2.5 Hz, 1H), 5.63 (ddd, J = 10.5, 5.1, 2.4 Hz, 1H), 4.45 (dd, J = 50.8, 3.4 Hz, 1H), 4.01 (d, J = 13.0 Hz, 1H), 3.85 − 3.69 (m, 4H), 3.66 (q, J = 6.5, 6.0 Hz, 1H), 3.58 (t, J = 7.1 Hz, 2H), 3.44 (p, J = 7.6 Hz, 1H), 3.29 − 3.04 (m, 3H), 2.93 − 2.81 (m, 1H), 2.31 (d, J = 30.1 Hz, 1H), 1.58 − 1.32 (m, 2H). | 549.30 |
| 4-acryloyl-1-(3- ((4-amino-7- methyl-5-(4- phenoxyphenyl)- 7H-pyrrolo[2,3- d]pyrimidin-6- yl)ethynyl) cyclobutyl) piperazin-2- one | 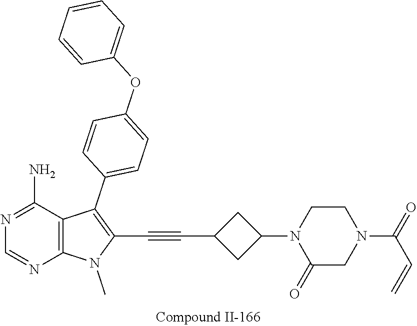 [SMILES: C=CC(=O)N1CCN(C2CC(C#Cc3c(-c4ccc(Oc5ccccc5)cc4)c4c(N)ncnc4n3C)C2)C(=O)C1] | ¹H NMR (400 MHz, DMSO-d₆) δ 8.18 (s, 1H), 7.60 − 7.48 (m, 2H), 7.48 − 7.37 (m, 2H), 7.15 (m, 5H), 6.75 (dd, J = 16.7, 10.4 Hz, 1H), 6.15 (dd, J = 16.6, 2.3 Hz, 1H), 5.72 (dd, J = 10.4, 2.3 Hz, 1H), 4.78 (p, J = 9.0 Hz, 1H), 4.21 (s, 1H), 4.06 (s, 1H), 3.77 (s, 1H), 3.72 (s, 4H), 3.38 (d, J = 16.6 Hz, 2H), 3.14 − 2.96 (m, 1H), 2.48(s, 2H), 2.28 (dt, J = 12.2, 9.8 Hz, 2H). | 546.43 |
| 1-(4-(3-((4-amino- 7-methyl-5-(4- phenoxyphenyl)- 7H-pyrrolo[2,3- d]pyrimidin-6- yl)ethynyl) cyclobutyl) piperazin-1- yl)prop-2-en-1-one | 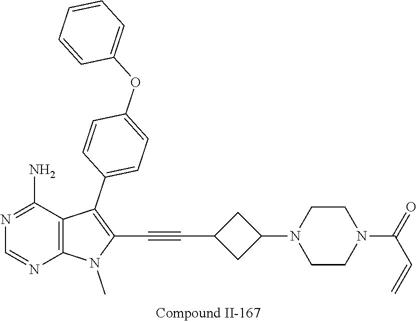 [SMILES: C=CC(=O)N1CCN(C2CC(C#Cc3c(-c4ccc(Oc5ccccc5)cc4)c4c(N)ncnc4n3C)C2)CC1] | ¹H NMR (400 MHz, DMSO-d₆) δ 8.19 (s, 1H), 7.52 (d, 2H), 7.43 (m, 2H), 6.82 − 6.75 (m, 1H), 6.25 (s, 2H), 6.11 (d, 1H), 5.68 (t, 1H), 3.72(s, 3H), 3.42 (m, 4H), 2.97 (m, 1H), 2.66 (m, 1H), 2.45 (m, 2H), 2.22 (m, 4H), 1.95 (d, 2H). | 533.3 |
| (R)-1-(4-(3-((4- amino-7-(1- hydroxypropan-2- yl)-5-(4- phenoxyphenyl)- 7H-pyrrolo[2,3- d]pyrimidin-6- yl)ethynyl)azetidin- 1-yl)piperidin-1- yl)prop-2-en-1-one | 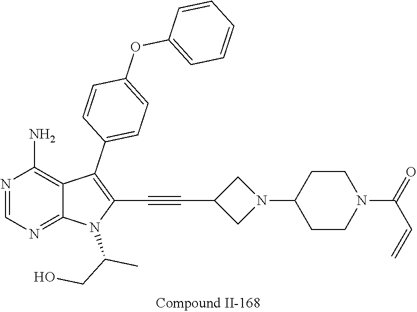 [SMILES: C=CC(=O)N1CCC(N2CC(C#Cc3c(-c4ccc(Oc5ccccc5)cc4)c4c(N)ncnc4n3[C@H](C)CO)C2)CC1] | ¹H NMR (400 MHz, Methanol-d₄) δ 8.16 (s, 1H), 7.57 − 7.55 (m, 2H), 7.43 − 7.40 (m, 2H), 7.20 − 7.11 (m, 5H), 6.76 (dd, J = 16.8, 10.7 Hz, 1H), 6.18 (dd, J = 16.8, 2.0 Hz, 1H), 5.73 (dd, J = 10.6, 2.0 Hz, 1H), 5.03 (td, J = 7.5, 5.1 Hz, 1H), 4.41 − 4.21 (m, 2H), 4.01 (dt, J = 11.5, 5.0 Hz, 2H), 3.66 (t, J = 7.4 Hz, 2H), 3.48 (p, J = 7.6 Hz, 1H), 3.17 (t, J = 7.3 Hz, 3H), 2.90 (t, J = 12.1 Hz, 1H), 2.38 (tt, J = 9.3, 3.8 Hz, 1H), 1.78 (d, J = 12.8 Hz, 2H), 1.62 (d, J = 7.0 Hz, 3H), 1.17 (t, J = 10.6 Hz, 2H). | 577.25 |
| (S)-1-(4-(3-((4- amino-7-(1- hydroxypropan-2- yl)-5-(4- phenoxyphenyl)- 7H-pyrrolo[2,3- d]pyrimidin-6- yl)ethynyl)azetidin- 1-yl)piperidin-1- yl)prop-2-en-1-one | 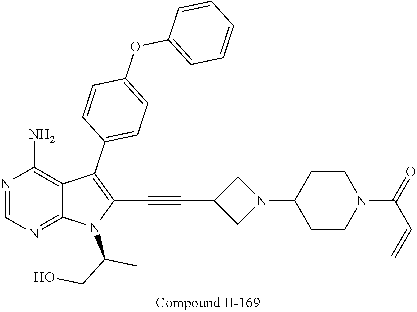 [SMILES: C=CC(=O)N1CCC(N2CC(C#Cc3c(-c4ccc(Oc5ccccc5)cc4)c4c(N)ncnc4n3[C@@H](C)CO)C2)CC1] | ¹H NMR (400 MHz, Methanol-d₄) δ 8.16 (s, 1H), 7.56 − 7.54 (m, 2H), 7.41 (t, J = 7.9 Hz, 2H), 7.14 (ddd, J = 17.8, 15.4, 7.7 Hz, 5H), 6.76 (dd, J = 16.8, 10.7 Hz, 1H), 6.18 (dd, J = 16.8, 2.0 Hz, 1H), 5.73 (dd, J = 10.7, 2.0 Hz, 1H), 5.03 (td, J = 7.5, 5.1 Hz, 1H), 4.43 − 4.21 (m, 2H), 4.01 (dt, J = 11.4, 5.4 Hz, 2H), 3.66 (t, J = 7.3 Hz, 2H), 3.47 (p, J = 7.6 Hz, 1H), 3.18 (q, J = 7.3, 6.2 Hz, 3H), 2.90 (t, J = 12.1 Hz, 1H), 2.37 (dq, J = 9.5, 4.9, 3.9 Hz, 1H), 1.77 (d, J = 12.8 Hz, 2H), 1.62 (d, J = 7.1 Hz, 3H), 1.17 (t, J = 11.1 Hz, 2H). | 577.30 |
| 1-((3R,4S)-4-(3- ((4-amino-7- methyl-5-(4- phenoxyphenyl)- 7H-pyrrolo[2,3- d]pyrimidin-6- yl)ethynyl)azetidin- 1-yl)-3- fluoropiperidin-1- yl)prop-2-en-1-one | 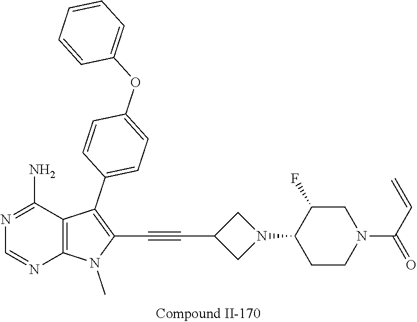 [SMILES: C=CC(=O)N1CC[C@H](N2CC(C#Cc3c(-c4ccc(Oc5ccccc5)cc4)c4c(N)ncnc4n3C)C2)[C@H](F)C1] | ¹H NMR (400 MHz, DMSO-d₆) δ 8.19 (s, 1H), 7.52 (d, J = 8.3 Hz, 2H), 7.43 (t, J = 7.7 Hz, 2H), 7.18 (t, J = 7.3 Hz, 1H), 7.13 (dd, J = 8.6, 3.3 Hz, 4H), 6.78 (ddd, J = 21.0, 16.6, 10.4 Hz, 1H), 6.24 (s, 1H), 6.09 (dd, J = 16.8, 2.4 Hz, 1H), 5.67 (d, J = 10.0 Hz, 1H), 4.71- 4.59 (s, 1H), 4.38 − 3.89 (d, J = 13.2 Hz, 2H), 3.72 (s, 3H), 3.63 − 3.55 (m, 2H), 3.47 (p, J = 7.5 Hz, 1H), 3.27 − 2.77 (t, J = 12.3 Hz, 4H), 2.47 − 2.41 (t, J = 12.5 Hz, 1H), 1.52 (d, J = 10.1 Hz, 1H), 1.42 − 1.21 (m, 1H). | 551.3 |
| 1-((3S,4R)-4-(3- ((4-amino-7- methyl-5-(4- phenoxyphenyl)- 7H-pyrrolo[2,3- d]pyrimidin-6- yl)ethynyl)azetidin- 1-yl)-3- fluoropiperidin-1- yl)prop-2-en-1-one | 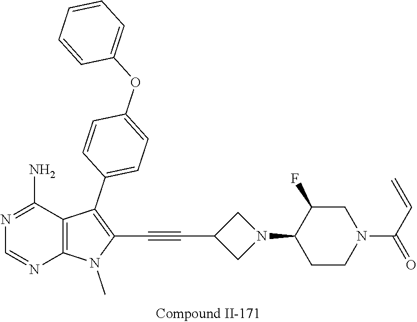 [SMILES: C=CC(=O)N1CC[C@@H](N2CC(C#Cc3c(-c4ccc(Oc5ccccc5)cc4)c4c(N)ncnc4n3C)C2)[C@@H](F)C1] | ¹H NMR (400 MHz, DMSO-d₆) δ 8.19 (s, 1H), 7.52 (d, J = 8.3 Hz, 2H), 7.43 (t, J = 7.7 Hz, 2H), 7.18 (t, J = 7.3 Hz, 1H), 7.13 (dd, J = 8.6, 3.3 Hz, 4H), 6.78 (ddd, J = 21.0, 16.6, 10.4 Hz, 1H), 6.24 (s, 1H), 6.09 (dd, J = 16.8, 2.4 Hz, 1H), 5.67 (d, J = 10.0 Hz, 1H), 4.71-4.59 (s, 1H), 4.38 − 3.89 (d, J = 13.2 Hz, 2H), 3.72 (s, 3H), 3.63 − 3.55 (m, 2H), 3.47 (p, J = 7.5 Hz, 1H), 3.27 − 2.77 (t, J = 12.3 Hz, 4H), 2.47 − 2.41 (t, J = 12.5 Hz, 1H), 1.52 (d, J = 10.1 Hz, 1H), 1.42 − 1.21 (m, 1H). | 551.3 |
| (E)-1-(3-((4- amino-7-methyl-5- (4- phenoxyphenyl)- 7H-pyrrolo [2,3- d]pyrimidin-6- yl)ethynyl)azetidin- 1-yl)-4-(3- fluoroazetidin-1- yl)but-2-en-1-one | 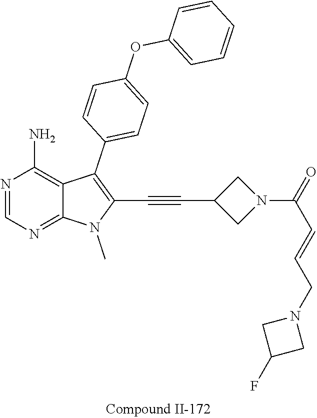 [SMILES: Cn1c(C#CC2CN(C(=O)/C=C/CN3CC(F)C3)C2)c(-c2ccc(Oc3ccccc3)cc2)c2c(N)ncnc21] | ¹H NMR (400 MHz, DMSO-d₆) δ 8.19 (s, 1H), 7.55 − 7.49 (m, 2H), 7.43 (t, J = 7.8 Hz, 2H), 7.22 − 7.08 (m, 5H), 6.53 (dt, J = 15.3, 5.2 Hz, 1H), 6.25 (s, 1H), 6.01 (d, J = 15.4 Hz, 1H), 5.08 (d, 1H), 4.52 (t, J = 8.4 Hz, 1H), 4.28 − 4.15 (m, 2H), 3.91 − 3.76 (m, 2H), 3.74 (s, 3H), 3.57 (dt, J = 15.7, 7.3 Hz, 2H), 3.21 (dd, J = 5.4, 1.7 Hz, 2H), 3.14 (s, 1H), 3.08 (s, 1H). | 537.3 |
| (E)-1-(6-((4- amino-7-methyl-5- (4- phenoxyphenyl)- 7H-pyrrolo[2,3- d]pyrimidin-6- yl)ethynyl)-2- azaspiro[3.3]heptan- 2-yl)-4- (azetidin-1-yl)but- 2-en-1-one | 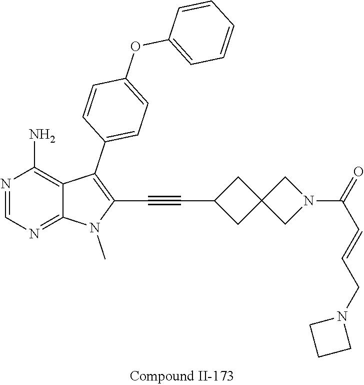 [SMILES: Cn1c(C#CC2CC3(C2)CN(C(=O)/C=C/CN2CCC2)C3)c(-c2ccc(Oc3ccccc3)cc2)c2c(N)ncnc21] | ¹H NMR (400 MHz, Methanol-d₄) δ 8.19 (d, J = 2.1 Hz, 1H), 7.55 (t, J = 8.7 Hz, 2H), 7.41 (t, J = 7.8 Hz, 2H), 7.22 − 7.07 (m, 5H), 6.70 − 6.59 (m, 1H), 6.04 (d, J = 16.1 Hz, 1H), 4.30 (s, 1H), 4.19 (s, 1H), 4.04 (s, 1H), 3.99 (s, 1H), 3.81 (s, 3H), 3.27 (m, 6.5 Hz, 6H), 2.67 (t, J = 10.1 Hz, 2H), 2.36 (t, 2H), 2.27 (d, J = 11.2 Hz, 1H), 2.15 (dt, J = 13.7, 7.1 Hz, 2H). | 559.25 |
| (E)-1-(6-((4- amino-7-methyl-5- (4- phenoxyphenyl)- 7H-pyrrolo[2,3- d]pyrimidin-6- yl)ethynyl)-2- azaspiro[3.3]heptan- 2-yl)-4-(3- hydroxyazetidin-1- yl)but-2-en-1-one | 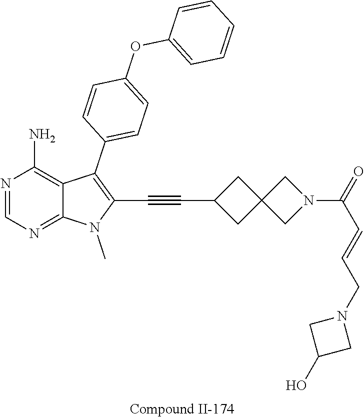 [SMILES: Cn1c(C#CC2CC3(C2)CN(C(=O)/C=C/CN2CC(O)C2)C3)c(-c2ccc(Oc3ccccc3)cc2)c2c(N)ncnc21] | ¹H NMR (400 MHz, Methanol-d4) δ 8.18 (d, J = 2.1 Hz, 1H), 7.54 (t, J = 8.8 Hz, 2H), 7.41 (t, J = 7.8 Hz, 2H), 7.22 − 7.06 (m, 5H), 6.73 − 6.60 (m, 1H), 6.06 (dd, J = 21.8, 15.4 Hz, 1H), 4.36 (p, J = 6.7 Hz, 1H), 4.30 (s, 1H), 4.18 (s, 1H), 4.04 (s, 1H), 3.99 (s, 1H), 3.80 (s, 3H), 3.67 (ddd, J = 12.8, 8.4, 6.1 Hz, 2H), 3.33 − 3.25 (m, 2H), 3.24 (s, 1H), 2.95 (td, J = 8.9, 6.2 Hz, 2H), 2.66 (t, J = 10.1 Hz, 2H), 2.40 − 2.30 (m, 2H). | 575.30 |
| (E)-1-(6-((4- amino-7-methyl-5- (4- phenoxyphenyl)- 7H-pyrrolo[2,3- d]pyrimidin-6- yl)ethynyl)-2- azaspiro[3.3]heptan- 2-yl)-4-(3- fluoroazetidin-1- yl)but-2-en-1-one | 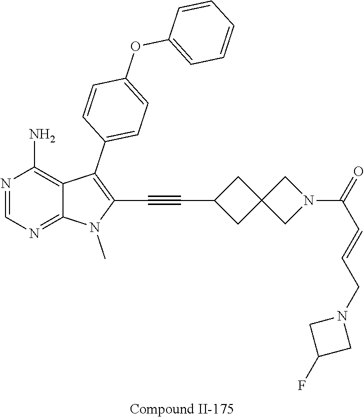 [SMILES: Cn1c(C#CC2CC3(C2)CN(C(=O)/C=C/CN2CC(F)C2)C3)c(-c2ccc(Oc3ccccc3)cc2)c2c(N)ncnc21] | ¹H NMR (400 MHz, Methanol-d4) δ 8.19 (s, 1H), 7.55 (t, 2H), 7.41 (t, 2H), 7.22 − 7.07 (m, 5H), 6.73 − 6.60 (m, 1H), 6.08 (dd, J = 21.6, 15.4 Hz, 1H), 5.23-5.08 (dt, J = 10.6, 5.2 Hz, 1H), 4.31 (s, 1H), 4.18 (s, 1H), 4.04 (s, 1H), 3.99 (s, 1H), 3.81 (s, 3H), 3.66 (tt, J = 16.1, 8.0 Hz, 2H), 3.36 (d, J = 5.6 Hz, 2H), 3.33 − 3.20 (m, 3H), 2.67 (t, J = 10.0 Hz, 2H), 2.41 − 2.24 (m, 2H). | 577.25 |
| 1-(4-(3-((4-amino- 7-methyl-5-(4- phenoxyphenyl)- 7H-pyrrolo[2,3- d]pyrimidin-6- yl)ethynyl)-3- fluoroazetidin-1- yl)piperidin-1- yl)prop-2-en-1-one | 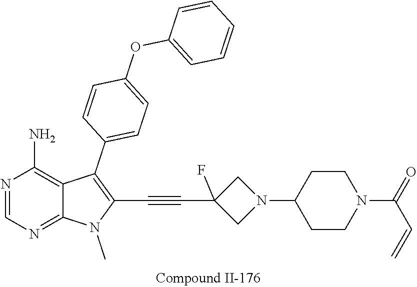 [SMILES: C=CC(=O)N1CCC(N2CC(F)(C#Cc3c(-c4ccc(Oc5ccccc5)cc4)c4c(N)ncnc4n3C)C2)CC1] | ¹H NMR (400 MHz, Methanol-d4) δ 8.22 (s, 1H), 7.57 (d, J = 8.3 Hz, 2H), 7.43 (t, J = 7.7 Hz, 2H), 7.21- 7.10 (m, 5H), 6.76 (dd, J = 16.8, 10.6 Hz, 1H), 6.18 (dd, J = 16.7, 1.9 Hz, 1H), 5.73 (dd, J = 10.7, 1.9 Hz, 1H), 4.32 (d, J = 13.3 Hz, 1H), 4.01 (d, J = 13.6 Hz, 1H), 3.85 (s, 3H), 3.73 (dd, J = 15.2, 8.9 Hz, 2H), 3.62 (dd, J = 19.3, 9.0 Hz, 2H), 3.22 (t, J = 12.2 Hz, 1H), 2.97 (t, J = 11.7 Hz, 1H), 2.56 − 2.46 (m, 1H), 1.79 (d, J = 12.7 Hz, 2H), 1.22 (d, J = 10.4 Hz, 2H). | 551.20 |
| (E)-7-methyl-5-(4- phenoxyphenyl)-6- ((1-(1-(prop-1- enylsulfonyl) piperidin- 4-yl)azetidin- 3-yl)ethynyl)-7H- pyrrolo[2,3- d]pyrimidin-4- amine | 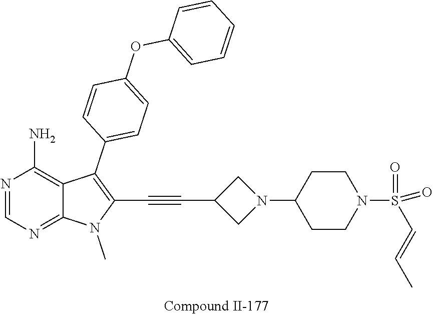 [SMILES: C/C=C/S(=O)(=O)N1CCC(N2CC(C#Cc3c(-c4ccc(Oc5ccccc5)cc4)c4c(N)ncnc4n3C)C2)CC1] | ¹H NMR (400 MHz, DMSO-d₆) δ 8.19 (s, 1H), 7.52 (d, J = 8.1 Hz, 2H), 7.43 (t, J = 7.7 Hz, 2H), 7.19 (t, J = 7.4 Hz, 1H), 7.15 − 7.09 (m, 4H), 6.62 (dq, J = 13.6, 6.7 Hz, 1H), 6.47 (d, J = 15.0 Hz, 1H), 6.25 (s, 2H), 3.72 (s, 3H), 3.54 (t, J = 6.9 Hz, 2H), 3.41 (p, J = 7.4 Hz, 1H), 3.23 (dt, J = 10.5, 4.6 Hz, 2H), 2.97 (t, J = 6.7 Hz, 2H), 2.72 (t, J = 10.2 Hz, 2H), 2.16 − 2.06 (m, 1H), 1.90 (d, J = 6.7 Hz, 3H), 1.64 (d, J = 13.0 Hz, 2H), 1.22 (d, J = 10.5 Hz, 2H). | 583.2 |
| 1-(4-((4-amino-7- methyl-5-(4- phenoxyphenyl)- 7H-pyrrolo[2,3- d]pyrimidin-6- yl)ethynyl)azepan- 1-yl)prop-2-en-1- one | 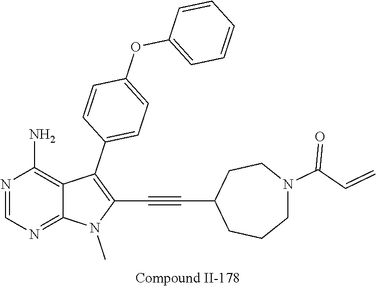 [SMILES: C=CC(=O)N1CCCC(C#Cc2c(-c3ccc(Oc4ccccc4)cc3)c3c(N)ncnc3n2C)CC1] | ¹H NMR (400 MHz, DMSO-d₆) δ 8.18 (s, 1H), 7.50 (d, J = 8.3 Hz, 2H), 7.42 (t, J = 7.7 Hz, 2H), 7.17 (t, J = 7.3 Hz, 1H), 7.11 (t, J = 7.7 Hz, 4H), 6.76 − 6.67 (m, 1H), 6.14 (s, 1H), 6.07 (ddd, J = 17.0, 7.2, 2.5 Hz, 1H), 5.66 − 5.57 (m, 1H), 3.66 − 3.52 (m, 2H), 3.50 − 3.39 (m, 2H), 3.06 (s, 1H), 1.90 − 1.70 (m, 6H). | 492.20 |
| (E)-1-(4-((4- amino-7-methyl-5- (4- phenoxyphenyl)- 7H-pyrrolo[2,3- d]pyrimidin-6- yl)ethynyl)azepan- 1-yl)-4- (dimethylamino) but-2-en-1-one | 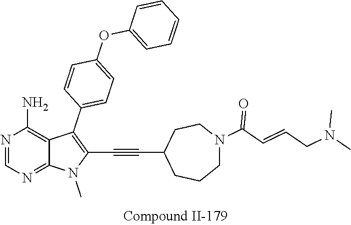 [SMILES: CN(C)C/C=C/C(=O)N1CCCC(C#Cc2c(-c3ccc(Oc4ccccc4)cc3)c3c(N)ncnc3n2C)CC1] | ¹H NMR (400 MHz, DMSO-d₆) δ 8.17 (s, 1H), 7.50 (dd, J = 8.7, 3.0 Hz, 2H), 7.42 (t, J = 7.8 Hz, 2H), 7.18 (d, J = 7.4 Hz, 1H), 7.11 (dd, J = 8.1, 5.9 Hz, 4H), 6.58 − 6.44 (m, 2H), 6.16 (s, 1H), 3.70 (s, 3H), 3.57 (s, 4H), 3.06 (s, 1H), 2.97 (d, J = 4.7 Hz, 1H), 2.95 − 2.83 (m, 1H), 2.10 (s, 3H), 2.04 (s, 3H), 1.88 − 1.68 (d, J = 6.8 Hz, 6H). | 549.3 |
| (E)-1-(4-((4- amino-7-methyl-5- (4- phenoxyphenyl)- 7H-pyrrolo[2,3- d]pyrimidin-6- yl)ethynyl)azepan- 1-yl)-4-(piperidin- 1-yl)but-2-en-1- one formate | 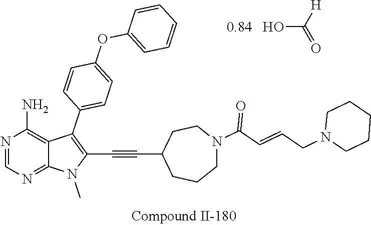 [SMILES: Cn1c(C#CC2CCCN(C(=O)/C=C/CN3CCCCC3)CC2)c(-c2ccc(Oc3ccccc3)cc2)c2c(N)ncnc21 ; O=CO] | 1H NMR (400 MHz, Methanol-d₄) δ 8.38 (s, 1H), 8.20 (d, J = 3.3 Hz, 1H), 7.55 − 7.49 (m, 2H), 7.46 − 7.36 (m, 2H), 7.22 − 7.10 (m, 3H), 7.09 (s, 1H), 7.08 − 7.05 (m, 1H), 6.87 (dd, J = 15.1, 8.6 Hz, 1H), 6.67 (tt, J = 15.2, 7.1 Hz, 1H), 4.82 (s, 1H), 3.94 − 3.78 (m, 4H), 3.75 − 3.50 (m, 5H), 3.07 (s, 5H), 2.08 − 1.80 (m, 9H), 1.64 (s, 4H). | 589.3 |
| (E)-1-(4-((4- amino-7-methyl-5- (4- phenoxyphenyl)- 7H-pyrrolo[2,3- d]pyrimidin-6- yl)ethynyl)azepan- 1-yl)-4- morpholinobut-2- en-1-one | 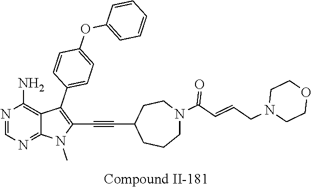 [SMILES: Cn1c(C#CC2CCCN(C(=O)/C=C/CN3CCOCC3)CC2)c(-c2ccc(Oc3ccccc3)cc2)c2c(N)ncnc21] | ¹H NMR (400 MHz, Methanol-d₄) δ 8.19 (d, J = 2.4 Hz, 1H), 7.53 (dd, J = 8.7, 1.5 Hz, 2H), 7.46 − 7.36 (m, 2H), 7.21 − 7.05 (m, 5H), 6.80 − 6.57 (m, 2H), 3.85 (m, 1H), 3.77 − 3.45 (m, 8H), 3.28 − 3.08 (m, 3H), 2.53 (d, J = 5.0 Hz, 2H), 2.44 (s, 2H), 2.08 − 1.94 (m, 2H), 1.94 − 1.68 (m, 4H). | 591.3 |
| (E)-1-(4-((4- amino-7-methyl-5- (4- phenoxyphenyl)- 7H-pyrrolo[2,3- d]pyrimidin-6- yl)ethynyl)azepan- 1-yl)-4-(azetidin- 1-yl)but-2-en-1- one | 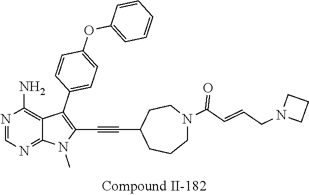 [SMILES: Cn1c(C#CC2CCCN(C(=O)/C=C/CN3CCC3)CC2)c(-c2ccc(Oc3ccccc3)cc2)c2c(N)ncnc21] | 1H NMR (400 MHz, Methanol-d4) δ 8.19 (d, J = 1.5 Hz, 1H), 7.54 (dd, J = 8.6, 2.4 Hz, 2H), 7.41 (t, J = 7.8 Hz, 2H), 7.19 − 7.08 (m, 5H), 6.62 − 6.51 (m, 2H), 3.81 (s, 3H), 3.68 (s, 1H), 3.59 (d, J = 5.7 Hz, 2H), 3.31 − 3.07 (m, 7H), 2.21 (d, J = 29.3 Hz, 1H), 2.16 − 1.71 (m, 8H). | 561.30 |
| (E)-1-(4-((4- amino-7-methyl-5- (4- phenoxyphenyl)- 7H-pyrrolo[2,3- d]pyrimidin-6- yl)ethynyl)azepan- 1-yl)-4-(3- hydroxyazetidin-1- yl)but-2-en-1-one | 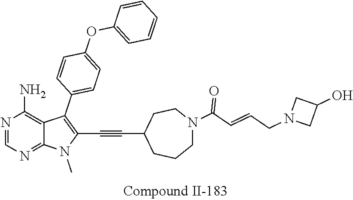 [SMILES: Cn1c(C#CC2CCCN(C(=O)/C=C/CN3CC(O)C3)CC2)c(-c2ccc(Oc3ccccc3)cc2)c2c(N)ncnc21] | ¹H NMR (400 MHz, Methanol-d₄) δ 8.19 (d, J = 1.8 Hz, 1H), 7.54 (d, J = 8.1 Hz, 2H), 7.42 (t, J = 7.7 Hz, 2H), 7.19-7.08 (m, 5H), 6.62 (tt, J = 15.8, 5.5 Hz, 1H), 6.54 − 6.51 (m, 1H), 4.34 (dp, J = 18.3, 6.0 Hz, 1H), 3.81 (s, 3H), 3.71 − 3.51 (m, 6H), 3.28 (d, J = 5.8 Hz, 1H), 3.24 − 3.11 (m, 2H), 2.92 (dt, J = 20.9, 7.1 Hz, 2H), 2.03 − 1.77 (m, 6H). | 577.25 |
| (E)-1-(4-((4- amino-7-methyl-5- (4- phenoxyphenyl)- 7H-pyrrolo[2,3- d]pyrimidin-6- yl)ethynyl)azepan- 1-yl)-4-(3- fluoroazetidin-1- yl)but-2-en-1-one | 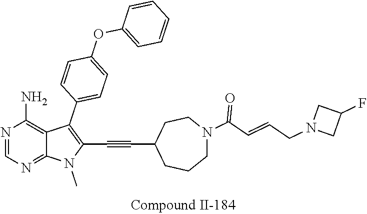 [SMILES: Cn1c(C#CC2CCCN(C(=O)/C=C/CN3CC(F)C3)CC2)c(-c2ccc(Oc3ccccc3)cc2)c2c(N)ncnc21] | ¹H NMR (400 MHz, Methanol-d₄) δ 8.19 (d, J = 1.7 Hz, 1H), 7.54 (dd, J = 8.7, 2.1 Hz, 2H), 7.41 (t, J = 7.8 Hz, 2H), 7.19 − 7.08 (m, 5H), 6.63 − 6.49 (m, 2H), 5.24 − 4.99 (m, 1H), 3.81 (s, 3H), 3.69 (q, J = 7.6, 6.7 Hz, 2H), 3.61 (d, J = 18.1 Hz, 4H), 3.28 − 3.15 (m, 5H), 2.05 − 1.77 (m, 6H). | 579.25 |
| N-((R)-1-(4-((4- amino-5-(4- phenoxyphenyl)-7- ((S)- tetrahydrofuran-3- yl)-7H- pyrrolo[2,3- d]pyrimidin-6- yl)ethynyl)piperidin- 1-yl)-1- oxopropan-2- yl)acrylamide | 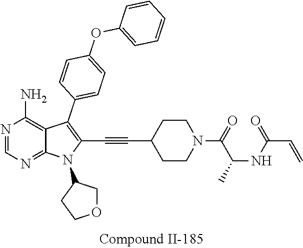 | ¹H NMR (400 MHz, DMSO-d₆) δ 8.42 (t, J = 7.2 Hz, 1H), 8.18 (s, 1H), 7.47 (dt, J = 33.0, 7.2 Hz, 4H), 7.21 − 7.07 (m, 5H), 6.31 (dd, J = 17.1, 10.2 Hz, 1H), 6.09 (d, J = 17.1 Hz, 2H), 5.63 − 5.46 (m, 2H), 4.83 (s, 1H), 4.19 (q, J = 7.7 Hz, 1H), 4.06 (d, J = 7.3 Hz, 2H), 3.92 (d, J = 7.8 Hz, 1H), 3.70 (q, J = 7.7 Hz, 1H), 3.51 (s, 1H), 3.45 (s, 1H), 3.30 − 3.10 (m, 1H), 3.04 (s, 1H), 2.62 (s, 1H), 2.35 (s, 1H), 1.81 (s, 2H), 1.54 (s, 2H), 1.17 (dd, J = 6.8, 2.7 Hz, 3H). | 605.3 |
| 1-((2R)-4-(3-((4- amino-7-methyl-5- (4- phenoxyphenyl)- 7H-pyrrolo[2,3- d]pyrimidin-6- yl)ethynyl)azetidin- 1-yl)-2- (methoxymethyl)p iperidin-1-yl)prop- 2-en-1-one | 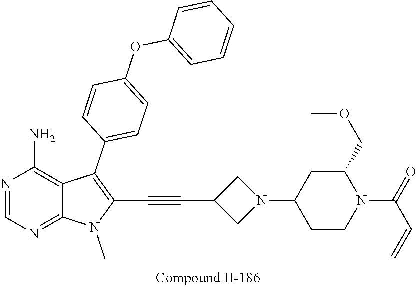 [SMILES: C=CC(=O)N1CCC(N2CC(C#Cc3c(-c4ccc(Oc5ccccc5)cc4)c4c(N)ncnc4n3C)C2)C[C@@H]1COC] | ¹H NMR (400 MHz, Chloroform-d) δ 8.35 (s, 1H), 7.53 (d, J = 8.4 Hz, 2H), 7.42 (t, J = 7.7 Hz, 2H), 7.20 (t, J = 7.4 Hz, 1H), 7.13 (dd, J = 8.2, 4.4 Hz, 4H), 6.63 (dd, J = 16.9, 10.6 Hz, 1H), 6.28 (dd, J = 16.9, 1.9 Hz, 1H), 5.71 − 5.63 (m, 1H), 5.51 (s, 1H), 4.29 (s, 1H), 3.86 (s, 3H), 3.79 (t, J = 8.5 Hz, 1H), 3.66 (dd, J = 13.9, 5.9 Hz, 3H), 3.41 (t, J = 7.6 Hz, 1H), 3.35 (s, 3H), 3.10 (d, J = 7.1 Hz, 1H), 3.03 (d, J = 7.3 Hz, 1H), 2.50 (s, 1H), 2.00 (s, 2H), 1.77 (d, J = 14.8 Hz, 1H), 1.62 (d, J = 24.7 Hz, 3H). | 577.3 |
| (E)-1-(4-(3-((4- amino-7-methyl-5- (4- phenoxyphenyl)- 7H-pyrrolo[2,3- d]pyrimidin-6- yl)ethynyl)azetidin- 1-yl)piperidin-1- yl)-4-methoxybut- 2-en-1-one | 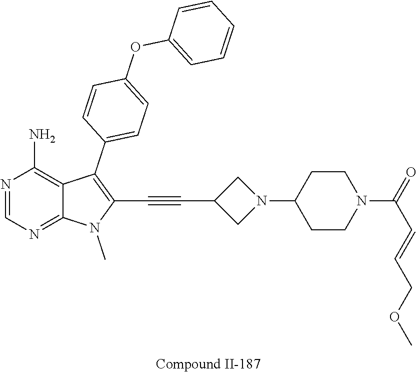 [SMILES: COC/C=C/C(=O)N1CCC(N2CC(C#Cc3c(-c4ccc(Oc5ccccc5)cc4)c4c(N)ncnc4n3C)C2)CC1] | ¹H NMR (400 MHz, DMSO-d₆) δ 8.18 (s, 1H), 7.64 (m, 1H), 7.54 (m, 2H), 7.43 (m, 2H), 7.15 (m, 5H), 6.64 (m, 2H), 6.25 (s, 2H), 4.04 (d, 2H), 3.94 (s, 1H), 3.75 (m, 1H), 3.72(s, 3H), 3.55(m, 2H), 3.43(m, 1H), 3.28(s, 3H), 3.15(s, 1H), 2.98(m, 3H), 2.25(s, 1H), 1.62(s, 2H), 1..05(s, 2H). | 577.4 |
| (1-(4-((4-amino-7- methyl-5-(4- phenoxyphenyl)- 7H-pyrrolo[2,3- d]pyrimidin-6- yl)ethynyl) piperidin-1-yl)-3- (dimethylamino) propan-1-one | 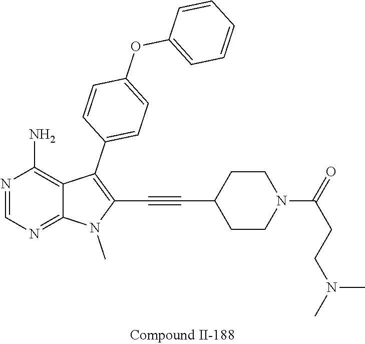 [SMILES: CN(C)CCC(=O)N1CCC(C#Cc2c(-c3ccc(Oc4ccccc4)cc3)c3c(N)ncnc3n2C)CC1] | ¹H NMR (400 MHz, Methanol-d₄) δ 8.20 (s, 1H), 7.58 − 7.53 (m, 2H), 7.46 − 7.39 (m, 2H), 7.21 − 7.07 (m, 5H), 3.82 (s, 3H), 3.75 − 3.62 (m, 2H), 3.59 − 3.45 (m, 2H), 3.12 − 3.05 (m, 1H), 2.89 (t, J = 7.1 Hz, 2H), 2.69 (t, J = 7.1 Hz, 2H), 2.48 (s, 6H), 2.00 − 1.85 (m, 2H), 1.70 (dd, J = 24.4, 13.5 Hz, 2H). | 523.3 |
| 1-(4-(3-((4-amino- 7-(2- methoxyethyl)-5-(4- phenoxyphenyl)- 7H-pyrrolo[2,3- d]pyrimidin-6- yl)ethynyl)azetidin- 1-yl)piperidin-1- yl)prop-2-en-1-one | 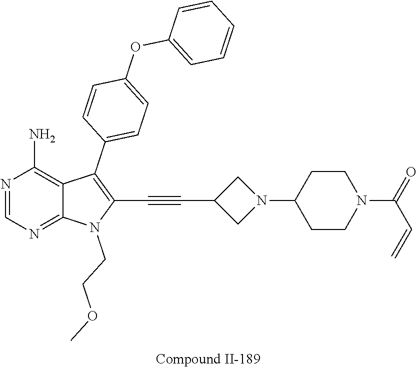 [SMILES: C=CC(=O)N1CCC(N2CC(C#Cc3c(-c4ccc(Oc5ccccc5)cc4)c4c(N)ncnc4n3CCOC)C2)CC1] | ¹H NMR (400 MHz, DMSO-d₆) δ 8.18 (s, 1H), 7.57 − 7.49 (m, 2H), 7.47 − 7.39 (m, 2H), 7.18 (t, J = 7.4 Hz, 1H), 7.17 − 7.09 (m, 4H), 6.78 (dd, J = 16.7, 10.5 Hz, 1H), 6.22 (s, 2H), 6.07 (dd, J = 16.7, 2.5 Hz, 1H), 5.65 (dd, J = 10.4, 2.4 Hz, 1H), 4.38 (t, J = 5.9 Hz, 2H), 3.96 (d, J = 13.2 Hz, 1H), 3.80 (d, J = 13.2 Hz, 1H), 3.74 (t, J = 5.9 Hz, 2H), 3.54 (t, J = 6.9 Hz, 2H), 3.41 (p, J = 7.3 Hz, 1H), 3.27 (s, 3H), 3.16 (t, J = 11.2 Hz, 1H), 3.00 (t, J = 6.6 Hz, 3H), 2.28 − 2.19 (m, 1H), 1.59 (s, 2H), 1.06 (s, 2H). | 577.3 |
| 1-(4-(3-((4-amino- 7-(2- hydroxyethyl)-5-(4- phenoxyphenyl)- 7H-pyrrolo[2,3- d]pyrimidin-6- yl)ethynyl)azetidin- 1-yl)piperidin-1- yl)prop-2-en-1-one | 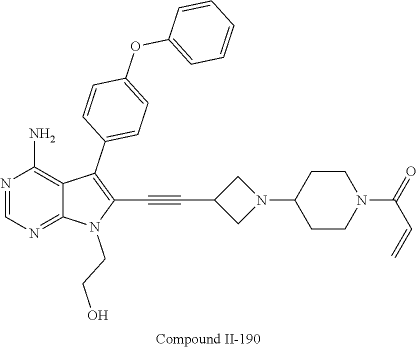 [SMILES: C=CC(=O)N1CCC(N2CC(C#Cc3c(-c4ccc(Oc5ccccc5)cc4)c4c(N)ncnc4n3CCO)C2)CC1] | ¹H NMR (400 MHz, Chloroform-d) δ 8.31 (s, 1H), 7.53 (d, J = 8.3 Hz, 2H), 7.41 (t, J = 7.8 Hz, 2H), 7.20 (t, J = 7.4 Hz, 1H), 7.13 (t, J = 7.6 Hz, 4H), 6.58 (dd, J = 16.8, 10.6 Hz, 1H), 6.27 (dd, J = 16.9, 1.9 Hz, 1H), 5.69 (dd, J = 10.6, 2.0 Hz, 1H), 5.46 (s, 2H), 4.48 (t, J = 4.7 Hz, 2H), 4.29 (d, J = 13.1 Hz, 1H), 4.06 (t, J = 4.8 Hz, 2H), 3.88 (d, J = 13.1 Hz, 1H), 3.68 (t, J = 7.1 Hz, 2H), 3.45 (p, J = 7.5 Hz, 1H), 3.14 (q, J = 9.2, 7.0 Hz, 3H), 3.04 (s, 1H), 2.38 − 2.29 (m, 1H), 1.72 (d, J = 12.7 Hz, 2H), 1.30 (ddt, J = 17.7, 13.4, 6.4 Hz, 2H). | 563.3 |
| (E)-1-(3-((4- amino-7-(2- methoxyethyl)-5-(4- phenoxyphenyl)- 7H-pyrrolo[2,3- d]pyrimidin-6- yl)ethynyl)azetidin- 1-yl)-4- (dimethylamino) but-2-en-1-one | 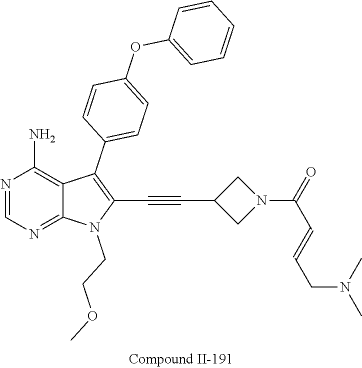 [SMILES: COCCn1c(C#CC2CN(C(=O)/C=C/CN(C)C)C2)c(-c2ccc(Oc3ccccc3)cc2)c2c(N)ncnc21] | ¹H NMR (400 MHz, DMSO-d₆) δ 8.19 (s, 1H), 7.57 − 7.49 (m, 2H), 7.48 − 7.39 (m, 2H), 7.18 (t, J = 7.4 Hz, 1H), 7.15 − 7.08 (m, 4H), 6.60 (dt, J = 15.4, 6.1 Hz, 1H), 6.26 (s, 2H), 6.08 (dt, J = 15.3, 1.6 Hz, 1H), 4.52 (t, J = 8.5 Hz, 1H), 4.40 (t, J = 5.8 Hz, 2H), 4.24 (t, J = 9.0 Hz, 1H), 4.20 − 4.13 (m, 1H), 3.91 − 3.84 (m, 1H), 3.84 − 3.74 (m, 1H), 3.74 (t, J = 5.7 Hz, 2H), 3.26 (s, 3H), 3.00 (dd, J = 6.1, 1.6 Hz, 2H), 2.13 (s, 6H). | 551.3 |
| (E)-4-(4-amino-6- ((1-(4- (dimethylamino) but-2- enoyl)azetidin-3- yl)ethynyl)-5-(4- phenoxyphenyl)- 7H-pyrrolo[2,3- d]pyrimidin-7-yl)- 1-methylpyrrolidin- 2-one | 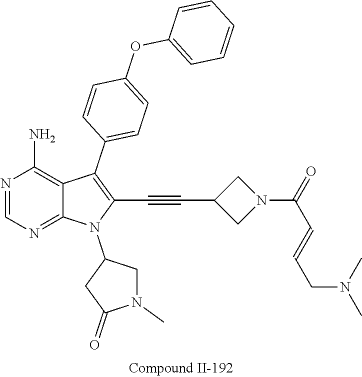 [SMILES: CN(C)C/C=C/C(=O)N1CC(C#Cc2c(-c3ccc(Oc4ccccc4)cc3)c3c(N)ncnc3n2C2CC(=O)N(C)C2)C1] | ¹H NMR (400 MHz, Methanol-d₄) δ 8.19 (s, 1H), 7.57 − 7.49 (m, 2H), 7.49 − 7.42 (m, 2H), 7.24 (s, 1H), 7.19 − 7.11 (m, 2H), 7.06 − 6.99 (m, 2H), 6.83 (dt, J = 15.4, 6.5 Hz, 1H), 6.21 (dt, J = 15.4, 1.6 Hz, 1H), 5.55 (tt, J = 9.0, 4.8 Hz, 1H), 4.64 (t, J = 8.7 Hz, 1H), 4.46 − 4.27 (m, 2H), 4.12 − 3.98 (m, 2H), 3.83 − 3.70 (m, 2H), 3.18 (dd, J = 6.6, 1.5 Hz, 2H), 3.12 − 3.01 (m, 1H), 2.96 (s, 3H), 2.85 (dd, J = 17.3, 5.1 Hz, 1H), 2.29 (s, 6H). | 590.45 |
| (E)-4-(4-amino-6- ((2-(4- (dimethylamino)but- 2-enoyl)-2- azaspiro[3.3]heptan- 6-yl)ethynyl)-5-(4- phenoxyphenyl)- 7H-pyrrolo[2,3- d]pyrimidin-7-yl)-1- methylpyrrolidin- 2-one | 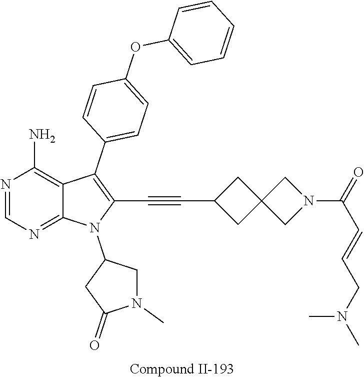 [SMILES: CN(C)C/C=C/C(=O)N1CC2(CC(C#Cc3c(-c4ccc(Oc5ccccc5)cc4)c4c(N)ncnc4n3C3CC(=O)N(C)C3)C2)C1] | ¹H NMR (400 MHz, Methanol-d₄) δ 8.19 (s, 1H), 7.56 − 7.48 (m, 2H), 7.43 − 7.36 (m, 2H), 7.24 (s, 1H), 7.18 − 7.11 (m, 2H), 7.01 (d, J = 8.7 Hz, 2H), 6.85 − 6.73 (m, 1H), 6.18 (dd, J = 15.4, 1.7 Hz, 1H), 5.55 (tt, J = 8.9, 4.8 Hz, 1H), 4.32 (d, J = 10.1 Hz, 2H), 4.06 (dd, J = 20.7, 8.2 Hz, 3H), 3.75 (dd, J = 10.7, 4.5 Hz, 1H), 3.24 − 3.13 (m, 3H), 3.07 (dd, J = 17.3, 9.0 Hz, 1H), 2.96 (s, 3H), 2.85 (dd, J = 17.3, 5.2 Hz, 1H), 2.66 (t, J = 9.9 Hz, 2H), 2.40 (dd, J = 10.8, 7.8 Hz, 2H), 2.29 (d, J = 2.4 Hz, 6H), 2.05 (s, 1H). | 630.4 |
| (E)-1-(3-((4- amino-7-(2- hydroxyethyl)-5- (4- phenoxyphenyl)- 7H-pyrrolo[2,3- d]pyrimidin-6- yl)ethynyl)azetidin- 1-yl)-4- (dimethylamino) but-2-en-1-one | 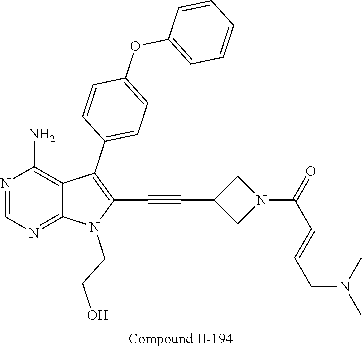 [SMILES: CN(C)C/C=C/C(=O)N1CC(C#Cc2c(-c3ccc(Oc4ccccc4)cc3)c3c(N)ncnc3n2CCO)C1] | ¹H NMR (400 MHz, Methanol-d₄) δ 8.19 (s, 1H), 7.60 − 7.53 (m, 2H), 7.42 (t, J = 7.8 Hz, 2H), 7.14 (ddd, J = 21.2, 13.7, 7.8 Hz, 5H), 6.82 (dt, J = 15.4, 6.5 Hz, 1H), 6.17 (d, J = 15.5 Hz, 1H), 4.62 (t, J = 8.7 Hz, 1H), 4.48 − 4.34 (m, 3H), 4.32 − 4.25 (m, 1H), 4.04 (dd, J = 9.9, 6.2 Hz, 1H), 3.94 (t, J = 5.9 Hz, 2H), 3.85 − 3.75 (m, 1H), 3.15 (d, J = 6.4 Hz, 2H), 2.28 (s, 6H). | 537.3 |
| 1-(3-(3-((4-amino- 7-methyl-5-(4- phenoxyphenyl)- 7H-pyrrolo[2,3- d]pyrimidin-6- yl)ethynyl)azetidin- 1-yl)pyrrolidin-1- yl)prop-2-en-1-one | 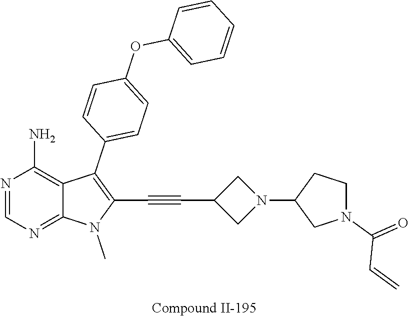 [SMILES: C=CC(=O)N1CCC(N2CC(C#Cc3c(-c4ccc(Oc5ccccc5)cc4)c4c(N)ncnc4n3C)C2)C1] | ¹H NMR (400 MHz, Chloroform-d) δ 8.35 (s, 1H), 7.52 (dd, J = 8.7, 2.0 Hz, 2H), 7.42 (t, J = 7.7 Hz, 2H), 7.25 − 7.07 (m, 5H), 6.49 − 6.35 (m, 2H), 5.72 − 5.65 (m, 1H), 5.51 (br, 1H), 3.86 (s, 3H), 3.74 − 3.56 (m, 4H), 3.55 − 3.41 (m, 3H), 3.14 − 2.95 (m, 3H), 1.98 − 1.78 (m, 2H). | 519.3 |
| N-(3-(4-((4- amino-7-methyl-5- (4-phenoxyphenyl)- 7H-pyrrolo[2,3- d]pyrimidin-6- yl)ethynyl)piperidine- 1-carbonyl)-1- methylazetidin-3- yl)acrylamide | 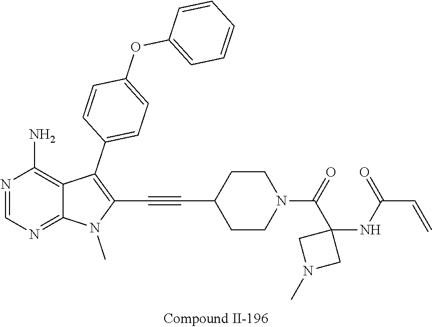 [SMILES: C=CC(=O)NC1(C(=O)N2CCC(C#Cc3c(-c4ccc(Oc5ccccc5)cc4)c4c(N)ncnc4n3C)CC2)CN(C)C1] | ¹H NMR (400 MHz, Chloroform-d) δ 8.36 (s, 1H), 7.51 (d, J = 8.6 Hz, 2H), 7.41 (dd, J = 8.5, 7.3 Hz, 2H), 7.22 − 7.07 (m, 6H), 6.32 (d, J = 16.5 Hz, 1H), 6.14 (dd, J = 16.9, 10.2 Hz, 1H), 5.69 (d, J = 10.2 Hz, 1H), 5.11 (s, 2H), 3.83 (s, 3H), 3.81 (s, 2H), 3.71 (s, 5H), 3.39 (s, 1H), 2.96 (s, 1H), 2.44 (s, 3H), 1.68 (s, 4H). | 590.4 |
| N-[(2R)-1-(4-[2- [4-amino-7- methyl-5-(4- phenoxyphenyl)- 7H-pyrrolo[2,3- d]pyrimidin-6- yllethynyl]piperidin- 1-yl)-3-hydroxy- 1-oxopropan-2- yl]prop-2-enamide | 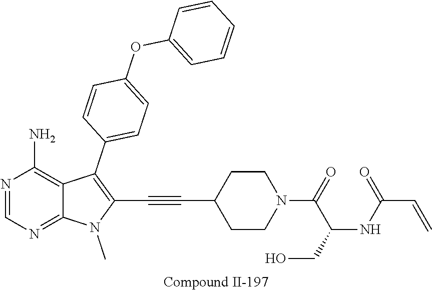 [SMILES: C=CC(=O)N[C@H](CO)C(=O)N1CCC(C#Cc2c(-c3ccc(Oc4ccccc4)cc3)c3c(N)ncnc3n2C)CC1] | ¹H NMR (400 MHz, DMSO-d₆) δ 8.34 (d, J = 8.2 Hz, 1H), 8.19 (s, 1H), 7.52 (d, J = 8.1 Hz, 2H), 7.48 − 7.39 (m, 2H), 7.24 − 7.07 (m, 5H), 6.36 (dd, J = 17.0, 10.2 Hz, 1H), 6.10 (d, J = 17.0 Hz, 1H), 5.59 (t, J = 10.7 Hz, 1H), 4.91 (m, J = 6.4 Hz, 2H), 3.79 − 3.66 (m, 5H), 3.60 (dt, J = 12.0, 6.4 Hz, 1H), 3.46 (dt, J = 11.0, 5.9 Hz, 2H), 3.29 (m, 1H), 3.04 (s, 1H), 1.83 (s, 2H), 1.55 (s, 2H). | 565.25 |
| N-[(2S)-1-(4-[2- [4-amino-7- methyl-5-(4- phenoxyphenyl)- 7H-pyrrolo[2,3- d]pyrimidin-6- yllethynyl]piperidin- 1-yl)-3-hydroxy- 1-oxopropan-2- yl]prop-2-enamide | 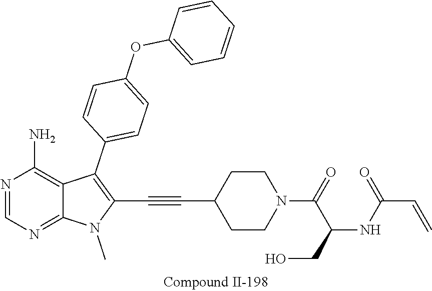 [SMILES: C=CC(=O)N[C@@H](CO)C(=O)N1CCC(C#Cc2c(-c3ccc(Oc4ccccc4)cc3)c3c(N)ncnc3n2C)CC1] | ¹H NMR (400 MHz, DMSO-d₆) δ 8.34 (d, J = 8.2 Hz, 1H), 8.19 (s, 1H), 7.52 (d, J = 8.1 Hz, 2H), 7.48 − 7.39 (m, 2H), 7.24 − 7.07 (m, 5H), 6.36 (dd, J = 17.0, 10.2 Hz, 1H), 6.10 (d, J = 17.0 Hz, 1H), 5.59 (t, J = 10.7 Hz, 1H), 4.91 (m, J = 6.4 Hz, 2H), 3.79 − 3.66 (m, 5H), 3.60 (dt, J = 12.0, 6.4 Hz, 1H), 3.46 (dt, J = 11.0, 5.9 Hz, 2H), 3.29 (m, 1H), 3.04 (s, 1H), 1.83 (s, 2H), 1.55 (s, 2H). | 565.25 |
| 1-((2S)-4-(3-((4- amino-7-methyl-5- (4-(6- methylpyridin-2- yloxy)phenyl)-7H- pyrrolo[2,3- d]pyrimidin-6- yl)ethynyl)azetidin- 1-yl)-2- (hydroxymethyl) piperidin-1-yl)prop- 2-en-1-one | 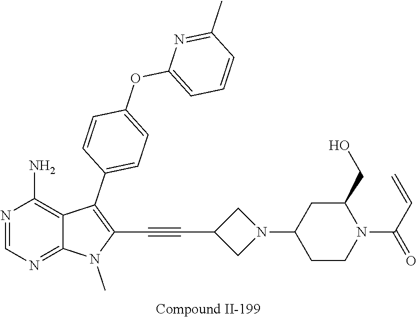 [SMILES: C=CC(=O)N1CCC(N2CC(C#Cc3c(-c4ccc(Oc5cccc(C)n5)cc4)c4c(N)ncnc4n3C)C2)C[C@H]1CO] | ¹H NMR (400 MHz, Chloroform-d) δ 8.36 (s, 1H), 7.65 (t, J = 7.8 Hz, 1H), 7.61 − 7.53 (m, 2H), 7.30 − 7.22 (m, 2H), 6.97 (d, J = 7.3 Hz, 1H), 6.76 (d, J = 8.1 Hz, 1H), 6.59 (dd, J = 16.8, 10.6 Hz, 1H), 6.27 (d, J = 16.8 Hz, 1H), 5.68 (dd, J = 10.6, 2.0 Hz, 1H), 5.55 (s, 1H), 4.72 (s, 1H), 3.87 (s, 4H), 3.79 − 3.66 (m, 5H), 3.49 (p, J = 7.6 Hz, 1H), 3.21 (dt, J = 22.2, 7.1 Hz, 2H), 2.63 − 2.57 (m, 1H), 2.51 (s, 3H), 1.93 (ddd, J = 14.9, 7.4, 3.8 Hz, 2H), 1.71 (s, 1H), 1.70 − 1.50 (m, 3H). | 578.3 |
| (R)-N-(1-(4-((4- amino-7-methyl-5- (4-((6- methylpyridin-2- yl)oxy)phenyl)- 7H-pyrrolo[2,3- d]pyrimidin-6- yl)ethynyl)piperidin- 1-yl)-3-hydroxy- 1-oxopropan-2- yl)acrylamide | 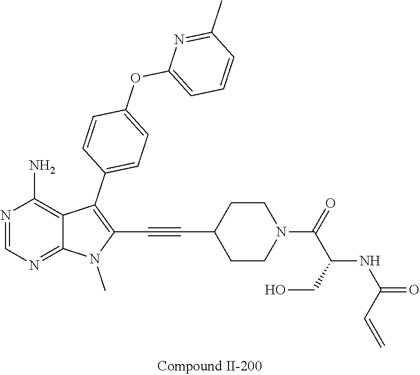 [SMILES: C=CC(=O)N[C@H](CO)C(=O)N1CCC(C#Cc2c(-c3ccc(Oc4cccc(C)n4)cc3)c3c(N)ncnc3n2C)CC1] | ¹H NMR (400 MHz, DMSO-d₆) δ 8.33 (d, J = 8.1 Hz, 1H), 8.20 (s, 1H), 7.76 (t, J = 7.7 Hz, 1H), 7.54 (d, J = 8.1 Hz, 2H), 7.24 (d, J = 8.3 Hz, 2H), 7.04 (d, J = 7.5 Hz, 1H), 6.83 (d, J = 8.1 Hz, 1H), 6.40 − 6.30 (m, 1H), 6.09 (d, J = 13.0 Hz, 1H), 5.58 (t, J = 11.3 Hz, 1H), 4.89 (s, 2H), 3.74 (s, 5H), 3.58 (s, 1H), 3.45 (dt, J = 11.2, 6.1 Hz, 2H), 3.30 − 3.19 (m, 1H), 3.05 (s, 1H), 2.37 (s, 3H), 1.85 (s, 2H), 1.56 (s, 2H). | 580.3 |
| 1-((3S,4R)-4-(3- ((4-amino-7- methyl-5-(4-(6- methylpyridin-2- yloxy)phenyl)-7H- pyrrolo[2,3- d]pyrimidin-6- yl)ethynyl)azetidin- 1-yl)-3- fluoropiperidin-1- yl)prop-2-en-1-one | 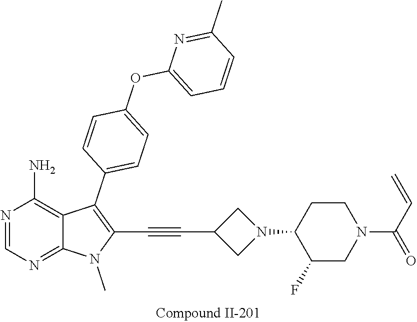 [SMILES: C=CC(=O)N1CC[C@@H](N2CC(C#Cc3c(-c4ccc(Oc5cccc(C)n5)cc4)c4c(N)ncnc4n3C)C2)[C@@H](F)C1] | ¹H NMR (400 MHz, DMSO-d₆) δ 8.20 (s, 1H), 7.76 (t, J = 7.8 Hz, 1H), 7.58 − 7.54 (m, 2H), 7.29 − 7.22 (m, 2H), 7.05 (d, J = 7.4 Hz, 1H), 6.85 (d, J = 8.2 Hz, 1H), 6.76 (dt, J = 17.0, 9.8 Hz, 1H), 6.20 (s, 1H), 6.08 (dd, J = 16.7, 2.4 Hz, 1H), 5.67 (d, J = 10.5 Hz, 1H), 4.71- 4.59 (s, 1H), 4.37 − 3.80 (m, 2H), 3.74 (s, 3H), 3.64 − 3.56 (m, 2H), 3.49 (p, J = 7.7 Hz, 1H), 3.14 (dd, J = 15.2, 8.4 Hz, 3H), 2.77 (s, 1H), 2.37 (s, 4H), 1.52 (s, 1H), 1.30 (d, J = 10.7 Hz, 1H). | 566.3 |
| 1-((3R,4S)-4-(3- ((4-amino-7- methyl-5-(4-(6- methylpyridin-2- yloxy)phenyl)-7H- pyrrolo[2,3- d]pyrimidin-6- yl)ethynyl)azetidin- 1-yl)-3- fluoropiperidin-1- yl)prop-2-en-1-one | 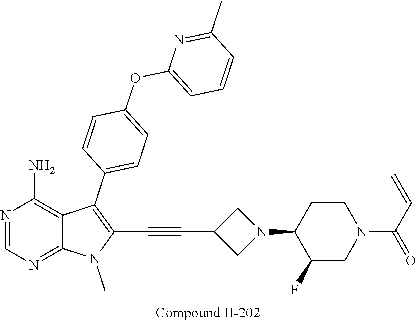 [SMILES: C=CC(=O)N1CC[C@H](N2CC(C#Cc3c(-c4ccc(Oc5cccc(C)n5)cc4)c4c(N)ncnc4n3C)C2)[C@H](F)C1] | ¹H NMR (400 MHz, DMSO-d₆) δ 8.20 (s, 1H), 7.76 (t, J = 7.8 Hz, 1H), 7.58 − 7.54 (m, 2H), 7.29 − 7.22 (m, 2H), 7.05 (d, J = 7.4 Hz, 1H), 6.85 (d, J = 8.2 Hz, 1H), 6.76 (dt, J = 17.0, 9.8 Hz, 1H), 6.20 (s, 1H), 6.08 (dd, J = 16.7, 2.4 Hz, 1H), 5.67 (d, J = 10.5 Hz, 1H), 4.71- 4.59 (s, 1H), 4.37 − 3.80 (m, 2H), 3.74 (s, 3H), 3.64 − 3.56 (m, 2H), 3.49 (p, J = 7.7 Hz, 1H), 3.25 (s, 1H), 3.14 (dd, J = 15.2, 8.4 Hz, 3H), 2.77 (s, 1H), 2.37 (s, 3H), 1.52 (s, 1H), 1.30 (d, J = 10.7 Hz, 1H). | 566.3 |
| 1-(4-[3-[2-(4- amino-7-methyl-5- [4-[(6- methylpyridin-2- yl)oxy]phenyl]- 7H-pyrrolo [2,3- d]pyrimidin-6- yl)ethynyl]-3- fluoroazetidin-1- yl]piperidin-1- yl)prop-2-en-1-one | 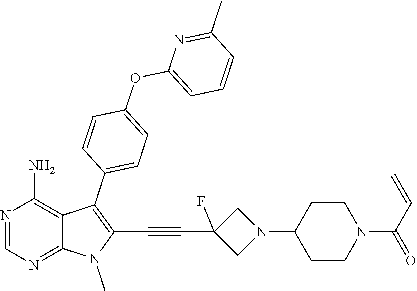 [SMILES: C=CC(=O)N1CCC(N2CC(F)(C#Cc3c(-c4ccc(Oc5cccc(C)n5)cc4)c4c(N)ncnc4n3C)C2)CC1] | ¹H NMR (400 MHz, DMSO-d₆) δ 8.24 (s, 1H), 7.76 (t, J = 7.7 Hz, 1H), 7.56 (d, J = 8.7 Hz, 2H), 7.26 (d, J = 8.7 Hz, 2H), 7.05 (d, J = 7.4 Hz, 1H), 6.86 (d, J = 8.1 Hz, 1H), 6.77 (dd, J = 16.7, 10.5 Hz, 1H), 6.06 (dd, J = 16.7, 2.5 Hz, 1H), 5.64 (dd, J = 10.4, 2.5 Hz, 1H), 4.01 (m, 1H), 3.85 (m, 1H), 3.77 (s, 3H), 3.64 (dd, J = 16.1, 8.6 Hz, 2H), 3.53 (dd, J = 19.8, 8.8 Hz, 2H), 3.22 − 3.10 (m, 1H), 2.95 (m, 1H), 2.37 (m, 4H), 1.63 (m, 2H), 1.09 (m, 2H). | 566.25 |
| | Compound II-203 | | |
| 1-((2S)-4-(3-((4- amino-7-methyl-5- (4-((6- methylpyridin-2- yl)oxy)phenyl)- 7H-pyrrolo[2,3- d]pyrimidin-6- yl)ethynyl)-3- fluoroazetidin-1- yl)-2- (hydroxymethyl) piperidin-1-yl)prop- 2-en-1-one | 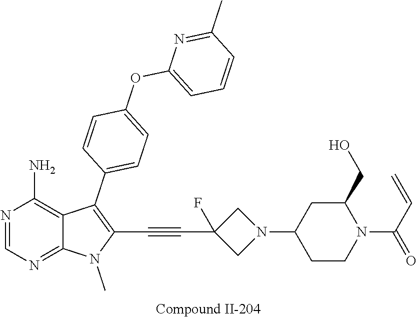 [SMILES: C=CC(=O)N1CCC(N2CC(F)(C#Cc3c(-c4ccc(Oc5cccc(C)n5)cc4)c4c(N)ncnc4n3C)C2)C[C@H]1CO] | ¹H NMR (400 MHz, Chloroform-d) δ 8.39 (s, 1H), 7.65 (t, J = 7.8 Hz, 1H), 7.63 − 7.55 (m, 2H), 7.30 − 7.23 (m, 2H), 6.97 (d, J = 7.4 Hz, 1H), 6.77 (d, J = 8.1 Hz, 1H), 6.58 (dd, J = 16.7, 10.8 Hz, 1H), 6.26 (d, J = 16.8 Hz, 1H), 5.67 (dd, J = 10.6, 1.9 Hz, 1H), 5.55 (s, 1H), 5.30 (s, 2H), 4.71 (s, 1H), 3.89 (s, 3H), 3.75 (s, 5H), 3.79 − 3.69 (m, 1H), 3.56 (td, J = 18.7, 18.0, 9.8 Hz, 2H), 2.68 (s, 1H), 2.50 (s, 3H), 1.92 (s, 2H), 1.70 (d, J = 15.1 Hz, 1H). | 596.30 |
| 1-((2S,4R)-4-(3- ((4-amino-7- methyl-5-(4- phenoxyphenyl)- 7H-pyrrolo[2,3- d]pyrimidin-6- yl)ethynyl)-3- fluoroazetidin-1- yl)-2- (hydroxymethyl) piperidin-1-yl)prop- 2-en-1-one | 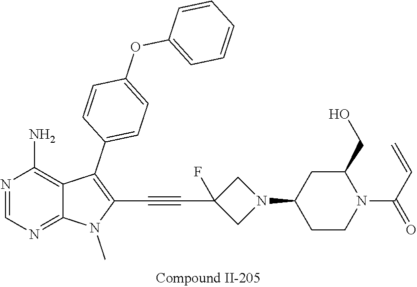 [SMILES: C=CC(=O)N1CC[C@@H](N2CC(F)(C#Cc3c(-c4ccc(Oc5ccccc5)cc4)c4c(N)ncnc4n3C)C2)C[C@H]1CO] | ¹H NMR (400 MHz, Chloroform-d) δ 8.38 (s, 1H), 7.56 (d, J = 8.3 Hz, 2H), 7.42 (t, J = 7.8 Hz, 2H), 7.24 − 7.09 (m, 5H), 6.58 (dd, J = 16.9, 10.5 Hz, 1H), 6.27 (d, J = 16.8 Hz, 1H), 5.69 (d, J = 10.6 Hz, 1H), 5.41 (s, 2H), 4.73 (s, 1H), 3.89 (s, 4H), 3.77 (dt, J = 21.6, 11.9 Hz, 4H), 3.57 (dt, J = 19.2, 9.9 Hz, 2H), 2.69 (s, 1H), 2.30 − 1.54 (m, 6H). | 581.3 |
| 1-[(1R,5S,6S)-6- [2-(4-amino-7- methyl-5-[4-[(6- methylpyridin-2- yl)oxy]phenyl]- 7H-pyrrolo[2,3- d]pyrimidin-6- yl)ethynyl]-3- azabicyclo[3.1.0] hexan-3-yl]prop-2- en-1-one | 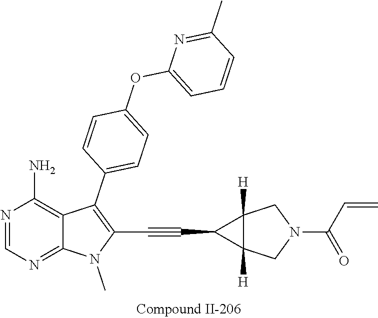 [SMILES: C=CC(=O)N1C[C@@H]2[C@@H](C#Cc3c(-c4ccc(Oc5cccc(C)n5)cc4)c4c(N)ncnc4n3C)[C@@H]2C1] | ¹H NMR (400 MHz, DMSO-d₆) δ 8.19 (s, 1H), 7.77 (t, 1H), 7.52 (d, J = 8.6 Hz, 2H), 7.25 (d, J = 8.6 Hz, 2H), 7.05 (d, J = 7.4 Hz, 1H), 6.87 (d, J = 8.2 Hz, 1H), 6.50 (dd, J = 16.8, 10.3 Hz, 1H), 6.30-6.14(br, 1H), 6.11 (dd, J = 16.7, 2.4 Hz, 1H), 5.66 (dd, J = 10.3, 2.4 Hz, 1H), 3.90 (d, J = 10.8 Hz, 1H), 3.76 (d, J = 12.6 Hz, 1H), 3.72 (s, 3H), 3.64 (dd, J = 10.7, 4.2 Hz, 1H), 3.40 (dd, J = 12.6, 4.3 Hz, 1H), 2.38 (s, 3H), 2.13 (m, J = 7.5, 3.8 Hz, 1H), 2.11 − 2.03 (m, 1H), 1.50 (t, J = 3.5 Hz, 1H). | 491.15 |
| (2E)-1- [(1R,5S,6S)-6-[2- [4-amino-7- methyl-5-(4- phenoxyphenyl)- 7H-pyrrolo[2,3- d]pyrimidin-6- ylethynyl]-3- azabicyclo[3.1.0] hexan-3-yl]-4- (dimethylamino) but-2-en-1-one | 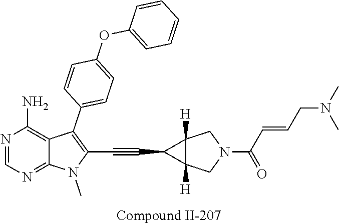 [SMILES: CN(C)C/C=C/C(=O)N1C[C@@H]2[C@@H](C#Cc3c(-c4ccc(Oc5ccccc5)cc4)c4c(N)ncnc4n3C)[C@@H]2C1] | ¹H NMR (400 MHz, DMSO-d₆) δ 8.18 (s, 1H), 7.50 − 7.42 (m, 4H), 7.20 − 7.12 (m, 5H), 6.62 − 6.55 (m, 1H), 6.28 (d, J = 15.2 Hz, 1H), 3.85 (d, J = 10.8 Hz, 1H), 3.75 (d, J = 12.6 Hz, 1H), 3.70 (s, 3H), 3.63 (dd, J = 10.8, 4.3 Hz, 1H), 3.38 (dd, J = 12.4, 4.2 Hz, 1H), 3.01 (d, J = 6.2 Hz, 2H), 2.14 (s, 6H), 2.10 (m, J = 7.3, 3.6 Hz, 1H), 2.04 (m, J = 3.7 Hz, 1H), 1.48 (s, 1H). | 533.20 |
| (2E)-1- [(1R,5S,6S)-6-[2- (4-amino-7- methyl-5-[4-[(6- methylpyridin-2- yl)oxy]phenyl]- 7H-pyrrolo[2,3- d]pyrimidin-6- yl)ethynyl]-3- azabicyclo[3.1.0] hexan-3-yl]-4- (dimethylamino) but-2-en-1-one | 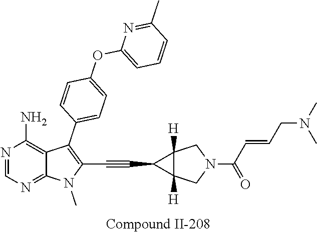 [SMILES: Cc1cccc(Oc2ccc(-c3c(C#C[C@@H]4[C@H]5CN(C(=O)/C=C/CN(C)C)C[C@@H]45)n(C)c4ncnc(N)c34)cc2)n1] | ¹H NMR (400 MHz, DMSO-d₆) δ 8.19 (s, 1H), 7.77 (t, J = 7.8 Hz, 1H), 7.51 (d, J = 8.6 Hz, 2H), 7.25 (d, J = 8.7 Hz, 2H), 7.05 (d, J = 7.4 Hz, 1H), 6.86 (d, J = 8.1 Hz, 1H), 6.58 (m, J = 15.2, 6.2 Hz, 1H), 6.28 (d, J = 15.3 Hz, 1H), 3.85 (d, J = 10.7 Hz, 1H), 3.75 (d, J = 12.6 Hz, 1H), 3.71 (s, 3H), 3.63 (dd, J = 10.6, 4.2 Hz, 1H), 3.38 (dd, 1H), 3.00 (dd, 2H), 2.38 (s, 3H), 2.14 (s, 6H), 2.11 (m, J = 7.5, 3.8 Hz, 1H), 2.05 (m, J = 7.5, 3.8 Hz, 1H), 1.49 (t, J = 3.4 Hz, 1H). | 548.40 |
| 1-((1R,5S,6s)-6- ((4-amino-5-(4- phenoxyphenyl)-7- (tetrahydrofuran- 3-yl)-7H- pyrrolo[2,3- d]pyrimidin-6- yl)ethynyl)-3-aza- bicyclo[3.1.0]hexan- 3-yl)prop-2-en- 1-one | 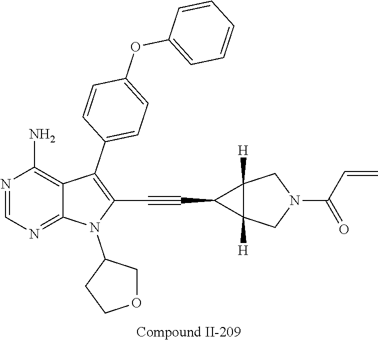 [SMILES: C=CC(=O)N1C[C@@H]2[C@@H](C#Cc3c(-c4ccc(Oc5ccccc5)cc4)c4c(N)ncnc4n3C3CCOC3)[C@@H]2C1] | ¹H NMR (400 MHz, DMSO-d₆) δ 8.18 (s, 1H), 7.55 − 7.37 (m, 4H), 7.26 − 7.10 (m, 4H), 6.50 (dd, J = 16.8, 10.4 Hz, 1H), 6.21 (s, 1H), 6.10 (dd, J = 16.7, 2.4 Hz, 1H), 5.65 (dd, J = 10.3, 2.4 Hz, 1H), 5.50 (p, J = 7.8 Hz, 1H), 4.30 − 4.14 (m, 1H), 4.14 − 3.99 (m, 2H), 3.90 (dd, J = 16.6, 9.2 Hz, 2H), 3.76 (d, J = 12.6 Hz, 1H), 3.63 (dd, J = 10.7, 4.2 Hz, 1H), 3.39 (dd, J = 12.6, 4.3 Hz, 1H), 2.60 (dt, J = 12.6, 7.2 Hz, 1H), 2.45 − 2.28 (m, 1H), 2.10 (dt, J = 7.5, 3.8 Hz, 1H), 2.03 (dt, J = 7.4, 3.8 Hz, 1H), 1.47 (t, J = 3.4 Hz, 1H). | 532.30 |
| N-[(2S)-1- [(1R,5S,6S)-6-[2- [4-amino-7- methyl-5-(4- phenoxyphenyl)- 7H-pyrrolo[2,3- d]pyrimidin-6- yl]ethynyl]-3- azabicyclo[3.1.0] hexan-3-yl]-3- hydroxy-1- oxopropan-2- yl]prop-2-enamide | 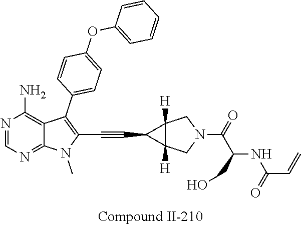 [SMILES: C=CC(=O)N[C@@H](CO)C(=O)N1C[C@@H]2[C@@H](C#Cc3c(-c4ccc(Oc5ccccc5)cc4)c4c(N)ncnc4n3C)[C@@H]2C1] | ¹H NMR (400 MHz, DMSO-d₆) δ 8.31 (t, J = 9.0 Hz, 1H), 8.18 (s, 1H), 7.55 − 7.41 (m, 4H), 7.25 − 7.10 (m, 5H), 6.34 (ddd, J = 15.8, 10.2, 4.8 Hz, 1H), 6.09 (d, J = 17.1 Hz, 1H), 5.60 (d, J = 10.2 Hz, 1H), 4.98 (m, J = 6.0 Hz, 1H), 4.69 − 4.47 (m, 1H), 3.98 (t, J = 11.1 Hz, 1H), 3.83 − 3.61 (m, 5H), 3.52 (ddt, J = 22.2, 10.8, 6.2 Hz, 2H), 3.29 (m, J = 4.2 Hz, 1H), 2.11 (s, 1H), 2.02 (s, J = 6.9 Hz, 1H), 1.41 (d, J = 56.0 Hz, 1H). | 563.20 |
| N-[2-[(1R,5S,6S)- 6-[2-[4-amino-7- methyl-5-(4- phenoxyphenyl)- 7H-pyrrolo[2,3- d]pyrimidin-6- yl]ethynyl]-3- azabicyclo[3.1.0] hexan-3-yl]-2- oxoethyl]prop-2- enamide | 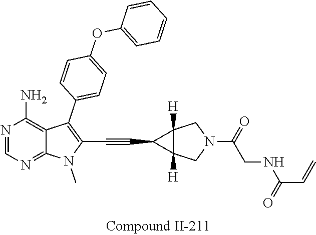 [SMILES: C=CC(=O)NCC(=O)N1C[C@@H]2[C@@H](C#Cc3c(-c4ccc(Oc5ccccc5)cc4)c4c(N)ncnc4n3C)[C@@H]2C1] | ¹H NMR (400 MHz, DMSO-d₆) δ 8.21 (t, J = 5.7 Hz, 1H), 8.18 (s, 1H), 7.69 − 7.53 (m, 1H), 7.53 − 7.40 (m, 4H), 7.24 − 7.10 (m, 5H), 6.38 (dd, J = 17.1, 10.2 Hz, 1H), 6.10 (d, J = 17.3 Hz, 1H), 5.60 (d, J = 10.2 Hz, 1H), 3.90 (t, J = 5.2 Hz, 2H), 3.72 (sd, J = 11.8 Hz, 5H), 3.59 (dd, J = 10.7, 4.2 Hz, 1H), 3.36 (dd, J = 4.4 Hz, 1H), 2.16 − 2.00 (m, 2H), 1.46 (t, J = 3.5 Hz, 1H). | 533.30 |
| N-[2-[(1R,5S,6S)- 6-[2-(4-amino-7- methyl-5-[4-[(6- methylpyridin-2- yl)oxy]phenyl]- 7H-pyrrolo[2,3- d]pyrimidin-6- yl)ethynyl]-3- azabicyclo[3.1.0] hexan-3-yl]-2- oxoethyl]prop-2- enamide | 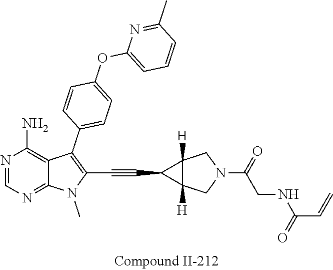 [SMILES: C=CC(=O)NCC(=O)N1C[C@@H]2[C@@H](C#Cc3c(-c4ccc(Oc5cccc(C)n5)cc4)c4c(N)ncnc4n3C)[C@@H]2C1] | ¹H NMR (400 MHz, DMSO-d₆) δ 8.22 (t, 1H), 8.20 (s, 1H), 7.78 (t, 1H), 7.52 (d, J = 8.6 Hz, 1H), 7.26 (d, J = 8.6 Hz, 2H), 7.05 (d, J = 7.3 Hz, 1H), 6.87 (d, J = 8.2 Hz, 1H), 6.38 (dd, J = 17.1, 10.2 Hz, 1H), 6.30-6.15 (br, 1H), 6.10 (dd, J = 17.1, 2.2 Hz, 1H), 5.60 (dd, J = 10.2, 2.2 Hz, 1H), 3.90 (dd, J = 5.5, 3.6 Hz, 2H), 3.73 (m, J = 8.1 Hz, 5H), 3.59 (dd, J = 10.6, 4.2 Hz, 1H), 3.34 (dd, J = 4.3 Hz, 1H), 2.38 (s, 3H), 2.14 (dt, J = 7.4, 3.8 Hz, 1H), 2.05 (dt, J = 7.5, 3.8 Hz, 1H), 1.49 (t, J = 3.5 Hz, 1H). | 548.25 |
| 4-(6-((1-(1- acryloylpiperidin- 4-yl)azetidin-3- yl)ethynyl)-4- amino-5-(4- phenoxyphenyl)- 7H-pyrrolo[2,3- d]pyrimidin-7-yl)- 1- methylpyrrolidin- 2-one | 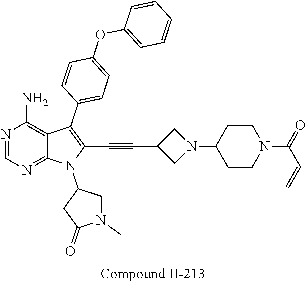 [SMILES: C=CC(=O)N1CCC(N2CC(C#Cc3c(-c4ccc(Oc5ccccc5)cc4)c4c(N)ncnc4n3C3CC(=O)N(C)C3)C2)CC1] | ¹H NMR (400 MHz, DMSO-d₆) δ 8.17 (s, 1H), 7.55 − 7.47 (m, 2H), 7.47 − 7.37 (m, 3H), 7.20 − 7.11 (m, 2H), 7.08 − 7.00 (m, 2H), 6.79 (dd, J = 16.7, 10.5 Hz, 1H), 6.22 (s, 2H), 6.07 (dd, J = 16.7, 2.5 Hz, 1H), 5.65 (dd, J = 10.4, 2.5 Hz, 1H), 5.54 − 5.43 (m, 1H), 4.00 (d, J = 12.9 Hz, 1H), 3.94 − 3.78 (m, 2H), 3.66 − 3.53 (m, 3H), 3.39 (p, J = 7.5 Hz, 1H), 3.18 (t, J = 11.2 Hz, 1H), 3.02 (t, J = 7.0 Hz, 3H), 2.88 (dd, J = 16.9, 8.9 Hz, 1H), 2.81 (s, 3H), 2.73 (dd, J = 16.9, 6.0 Hz, 1H), 2.34 − 2.25 (m, 1H), 1.62 (s, 2H), 1.08 (s, 2H). | 616.35 |
| 4-acryloyl-1- ((1s,3s)-3-((4- amino-7-methyl-5- (4- phenoxyphenyl)- 7H-pyrrolo[2,3- d]pyrimidin-6- yl)ethynyl)cyclobutyl) piperazin-2- one | 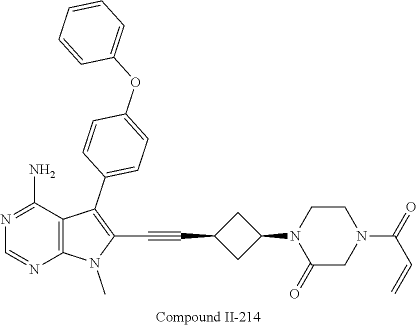 [SMILES: C=CC(=O)N1CCN([C@H]2C[C@@H](C#Cc3c(-c4ccc(Oc5ccccc5)cc4)c4c(N)ncnc4n3C)C2)C(=O)C1] | ¹H NMR (400 MHz, DMSO-d₆) δ 8.19 (s, 1H), 7.60 − 7.50 (m, 2H), 7.45 − 7.36 (m, 2H), 7.21 − 7.08 (m, 5H), 6.92 − 6.67 (m, 1H), 6.17 (d, J = 16.6 Hz, 2H), 5.74 (t, J = 8.9 Hz, 1H), 5.17 (p, J = 8.9 Hz, 1H), 4.20 (s, 1H), 4.06 (s, 1H), 3.82 (s, 1H), 3.75 (s, 4H), 3.43 (s, 2H), 3.21 (t, J = 9.6 Hz, 1H), 2.65 (q, J = 9.8 Hz, 2H), 2.18 (t, J = 9.4 Hz, 2H). | 546.63 |
| 1-(4-(3-((4-amino- 7-methyl-5-(4-(6- methylpyridin-2- yloxy)phenyl)-7H- pyrrolo[2,3- d]pyrimidin-6- yl)ethynyl)azetidin- 1-yl)-3- hydroxypiperidin- 1-yl)prop-2-en-1- one | 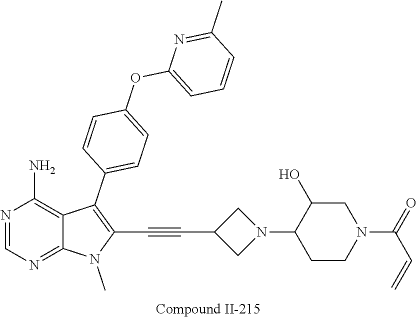 [SMILES: C=CC(=O)N1CCC(N2CC(C#Cc3c(-c4ccc(Oc5cccc(C)n5)cc4)c4c(N)ncnc4n3C)C2)C(O)C1] | ¹H NMR (400 MHz, Methanol-d₄) δ 8.20 (s, 1H), 7.76 (t, J = 7.8 Hz, 1H), 7.62 − 7.59 (m, 2H), 7.27 − 7.25 (m, 2H), 7.05 (d, J = 7.4 Hz, 1H), 6.83 (d, J = 8.2 Hz, 1H), 6.80 − 6.69 (m, 1H), 6.16 (dd, J = 16.8, 2.0 Hz, 1H), 5.72 (ddd, J = 10.5, 8.0, 2.0 Hz, 1H), 4.49 − 4.36 (m, 1H), 4.01 (dd, J = 27.0, 14.3 Hz, 1H), 3.83 (s, 3H), 3.80 − 3.65 (m, 3H), 3.51 (p, J = 7.7 Hz, 1H), 3.22 (dp, J = 7.8, 3.2, 2.4 Hz, 3H), 2.86 − 2.73 (m, 1H), 2.45 (s, 3H), 2.40 (q, J = 10.0, 9.3 Hz, 1H), 1.55 − 1.52 (m, 2H). | 564.30 |
| 1-(4-(3-((4-amino- 7-methyl-5-(4-(6- methylpyridin-2- yloxy)phenyl)-7H- pyrrolo[2,3- d]pyrimidin-6- yl)ethynyl)azetidin- 1-yl)-3- fluoropiperidin-1- yl)prop-2-en-1-one | 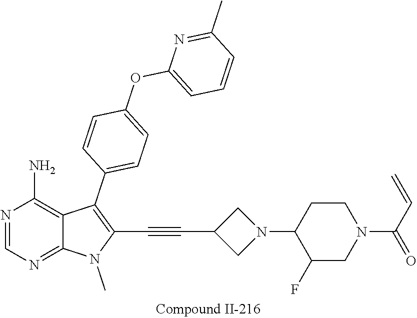 [SMILES: C=CC(=O)N1CCC(N2CC(C#Cc3c(-c4ccc(Oc5cccc(C)n5)cc4)c4c(N)ncnc4n3C)C2)C(F)C1] | ¹H NMR (400 MHz, DMSO-d₆) δ 8.20 (s, 1H), 7.76 (t, J = 7.8 Hz, 1H), 7.58 − 7.54 (m, 2H), 7.29 − 7.22 (m, 2H), 7.05 (d, J = 7.4 Hz, 1H), 6.85 (d, J = 8.2 Hz, 1H), 6.76 (dt, J = 17.0, 9.8 Hz, 1H), 6.20 (s, 1H), 6.08 (dd, J = 16.7, 2.4 Hz, 1H), 5.67 (d, J = 10.5 Hz, 1H), 4.71- 4.59 (s, 1H), 4.37 − 3.80 (m, 2H), 3.74 (s, 4H), 3.64 − 3.56 (m, 1H), 3.49 − 2.96 (m, 5H), 2.77 (s, 1H), 2.37 (s, 3H), 1.52 (s, 1H), 1.30 (d, J = 10.7 Hz, 1H). | 566.3 |
| 1-((3R,4R)-4-(3- ((4-amino-5-(3- methoxy-4-((6- methylpyridin-2- yl)oxy)phenyl)-7- methyl-7H- pyrrolo[2,3- d]pyrimidin-6- yl)ethynyl)azetidin- 1-yl)-3- hydroxypiperidin- 1-yl)prop-2-en-1- one | 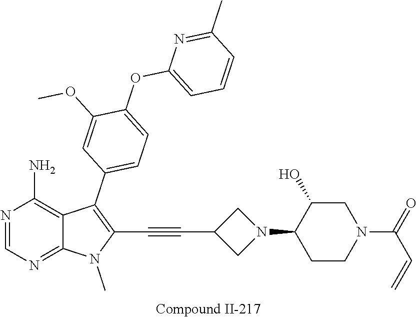 [SMILES: C=CC(=O)N1CC[C@@H](N2CC(C#Cc3c(-c4ccc(Oc5cccc(C)n5)c(OC)c4)c4c(N)ncnc4n3C)C2)[C@H](O)C1] | ¹H NMR (400 MHz, DMSO-d₆) δ 8.20 (s, 1H), 7.68 (t, J = 7.8 Hz, 1H), 7.31 − 7.21 (m, 2H), 7.12 (dd, J = 8.0, 1.9 Hz, 1H), 6.95 (d, J = 7.3 Hz, 1H), 6.81 − 6.66 (m, 2H), 6.09 − 5.98 (m, 1H), 5.62 (d, J = 10.5 Hz, 1H), 4.46 (d, J = 48.5 Hz, 1H), 4.00 (d, J = 12.9 Hz, 1H), 3.75 (d, J = 5.3 Hz, 7H), 3.63 − 3.43 (m, 4H), 3.19 − 3.03 (m, 4H), 2.84 (s, 1H), 2.33 (s, 3H), 2.27 (s, 1H), 1.33 (d, J = 18.9 Hz, 2H). | 593.69 |
| 1-((1R,5S,6s)-6- ((4-amino-5-(3- methoxy-4-((6- methylpyridin-2- yl)oxy)phenyl)-7- methyl-7H- pyrrolo[2,3- d]pyrimidin-6- yl)ethynyl)-3- azabicyclo[3.1.0] hexan-3-yl)prop-2- en-1-one | 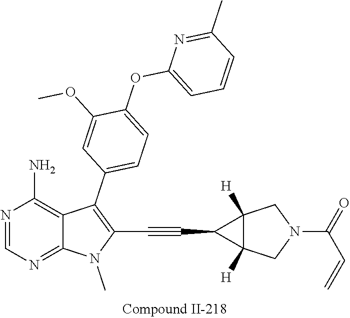 [SMILES: C=CC(=O)N1C[C@@H]2[C@@H](C#Cc3c(-c4ccc(Oc5cccc(C)n5)c(OC)c4)c4c(N)ncnc4n3C)[C@@H]2C1] | ¹H NMR (400 MHz, DMSO-d₆) δ 8.20 (s, 1H), 7.73 − 7.65 (m, 1H), 7.28 − 7.21 (m, 2H), 7.09 (dd, J = 8.1, 2.0 Hz, 1H), 6.96 (d, J = 7.3 Hz, 1H), 6.74 (d, J = 8.2 Hz, 1H), 6.50 (dd, J = 16.8, 10.3 Hz, 1H), 6.29 (br, 1H), 6.10 (dd, J = 16.7, 2.4 Hz, 1H), 5.66 (dd, J = 10.3, 2.4 Hz, 1H), 3.89 (d, J = 10.8 Hz, 1H), 3.76 (s, 3H), 3.74 (s, 3H), 3.64 (dd, J = 10.7, 4.2 Hz, 1H), 3.40 (dd, J = 12.6, 4.3 Hz, 1H), 2.33 (s, 3H), 2.13 (dt, J = 7.5, 3.8 Hz, 1H), 2.05 (dt, J = 7.4, 3.8 Hz, 1H), 1.54 (t, J = 3.5 Hz, 1H). | 521.25 |
| 1-((1R,5S,6s)-6- ((4-amino-7- isopropyl-5-(3- methoxy-4-((6- methylpyridin-2- yl)oxy)phenyl)- 7H-pyrrolo[2,3- d]pyrimidin-6- yl)ethynyl)-3- azabicyclo[3.1.0] hexan-3-yl)prop-2- en-1-one | 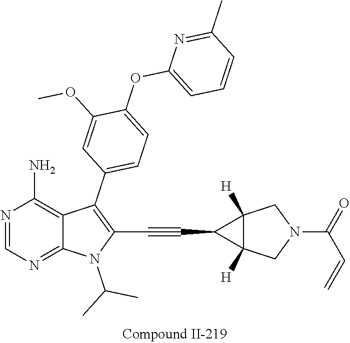 [SMILES: C=CC(=O)N1C[C@@H]2[C@@H](C#Cc3c(-c4ccc(Oc5cccc(C)n5)c(OC)c4)c4c(N)ncnc4n3C(C)C)[C@@H]2C1] | ¹H NMR (500 MHz, DMSO-d₆): δ 8.06-8.20 (m, 1H), 7.60-7.74 (m, 1H), 7.17-7.29 (m, 2H), 7.00-7.11 (m, 1H), 6.89-6.97 (m, 1H), 6.66- 6.77 (m, 1H), 6.42-6.55 (m, 1H), 6.00-6.15 (m, 1H), 5.57-5.70 (m, 1H), 5.01-5.15 (m, 1H), 3.84-3.91 (m, 1H), 3.73-3.78 (m, 3H), 3.57- 3.65 (m, 1H), 3.34-3.43 (m, 1H), 2.27-2.36 (m, 3H), 1.91-2.11 (m, 2H), 1.55-1.70 (m, 6H), 1.19-1.29 (m, 1H). | 549 |
| 1-((3S,4S)-4-(3- ((4-amino-7- methyl-5-(4-((6- methylpyridin-2- yl)oxy)phenyl)- 7H-pyrrolo[2,3- d]pyrimidin-6- yl)ethynyl)-3- fluoroazetidin-1- yl)-3- hydroxypiperidin- 1-yl)prop-2-en-1- one | 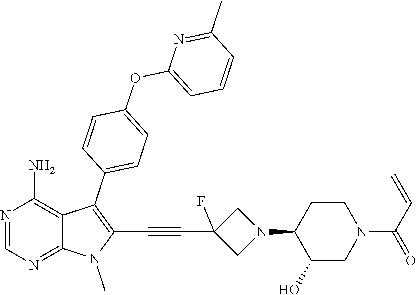 [SMILES: C=CC(=O)N1CC[C@H](N2CC(F)(C#Cc3c(-c4ccc(Oc5cccc(C)n5)cc4)c4c(N)ncnc4n3C)C2)[C@@H](O)C1] | ¹H NMR (400 MHz, DMSO-d₆) δ 8.23 (s, 1H), 7.76 − 7.71 (m, 1H), 7.54 (d, J = 8.4 Hz, 2H), 7.24 (d, J = 7.4 Hz, 2H), 7.03 (d, J = 8.3 Hz, 1H), 6.78 (d, J = 13.8 Hz, 1H), 6.75 − 6.67 (m, 1H), 6.35 (s, 2H), 6.05 (dd, J = 16.8, 2.4 Hz, 1H), 5.63 (d, J = 10.5 Hz, 1H), 4.76 − 4.52 (m, 1H), 3.96 − 3.62 (m, 5H), 3.60 − 3.42 (m, 5H),3.24 (d, J = 13.2 Hz, 1H), 2.98 (t, J = 11.3 Hz, 1H), 2.45 (s, 1H), 2.35 (s, 3H), 1.49 (d, J = 58.6 Hz, 2H). | 582.3 |
| 1-((3S,4S)-4-(3- ((4-amino-7- methyl-5-(4- phenoxyphenyl)- 7H-pyrrolo[2,3- d]pyrimidin-6- yl)ethynyl)azetidin- 1-yl)-3- hydroxypiperidin- 1-yl)prop-2-en-1- one | 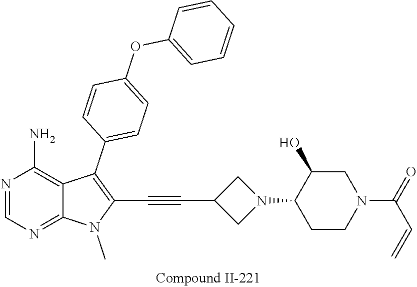 [SMILES: C=CC(=O)N1CC[C@H](N2CC(C#Cc3c(-c4ccc(Oc5ccccc5)cc4)c4c(N)ncnc4n3C)C2)[C@@H](O)C1] | ¹H NMR (400 MHz, DMSO-d₆) δ 8.19 (s, 1H), 7.52 (d, J = 8.2 Hz, 2H), 7.44 (t, J = 7.7 Hz, 2H), 7.16 (dd, J = 24.6, 7.8 Hz, 5H), 6.75 (td, J = 16.8, 10.5 Hz, 1H), 6.22 (s, 1H), 6.06 (d, J = 16.7 Hz, 1H), 5.64 (d, J = 10.6 Hz, 1H), 4.94 (d, J = 4.3 Hz, 1H), 3.93 − 3.62 (d, J = 6.8 Hz, 5H), 3.58 (d, J = 11.6 Hz, 1H), 3.44 (td, J = 16.7, 14.9, 8.8 Hz, 2H), 3.29 − 2.85 (m, 4H), 2.19 (s, 1H), 1.69 (s, 1H), 1.09 (s, 1H). | 549.3 |
| 1-((3R,4R)-3-(3- ((4-amino-7- methyl-5-(4- phenoxyphenyl)- 7H-pyrrolo[2,3- d]pyrimidin-6- yl)ethynyl)azetidin- 1-yl)-4- hydroxypiperidin- 1-yl)prop-2-en-1- one | 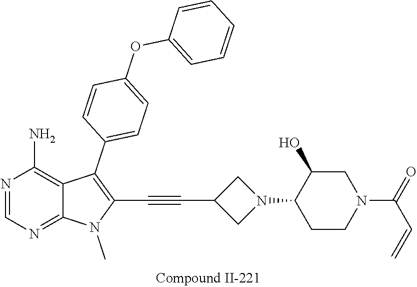 [SMILES: C=CC(=O)N1CC[C@H](N2CC(C#Cc3c(-c4ccc(Oc5ccccc5)cc4)c4c(N)ncnc4n3C)C2)[C@@H](O)C1] | ¹H NMR (400 MHz, DMSO-d₆) δ 8.19 (s, 1H), 7.52 (d, J = 8.3 Hz, 2H), 7.44 (t, J = 7.7 Hz, 2H), 7.18 (t, J = 7.3 Hz, 1H), 7.14 (d, J = 8.1 Hz, 4H), 6.77 (dt, J = 16.9, 11.9 Hz, 1H), 6.21 (s, 1H), 6.08 (dd, J = 16.6, 2.5 Hz, 1H), 5.65 (dd, J = 10.4, 2.5 Hz, 1H), 4.84 (dd, J = 9.3, 4.4 Hz, 1H), 3.72 (s, 3H), 3.65 − 3.47 (m, 5H), 3.42 (s, 2H), 3.24 (dd, J = 13.6, 6.0 Hz, 1H), 3.15 (s, 1H), 3.09 (t, J = 6.9 Hz, 1H), 2.05 (s, 1H), 1.79 − 1.67 (m, 1H), 1.33 (s, 1H). | 549.3 |
| (E)-1-((1R,5S,6s)- 6-((4-amino-7- methyl-5-(4- phenoxyphenyl)- 7H-pyrrolo[2,3- d]pyrimidin-6- yl)ethynyl)-3- azabicyclo[3.1.0] hexan-3-yl)-4- (diethylamino)but- 2-en-1-one | 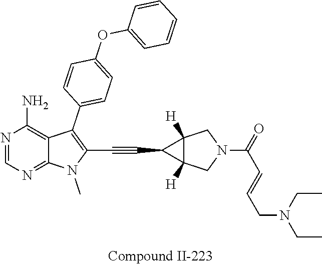 [SMILES: CCN(CC)C/C=C/C(=O)N1C[C@@H]2[C@@H](C#Cc3c(-c4ccc(Oc5ccccc5)cc4)c4c(N)ncnc4n3C)[C@@H]2C1] | ¹H NMR (400 MHz, Methanol-d₄) δ 8.18 (s, 1H), 7.55 − 7.47 (m, 2H), 7.46 − 7.37 (m, 2H), 7.23 − 7.08 (m, 5H), 6.85 (dt, J = 15.2, 6.6 Hz, 1H), 6.46 − 6.37 (m, 1H), 3.92 (dd, J = 14.6, 11.8 Hz, 2H), 3.79 (s, 3H), 3.75 (m, 1H),3.52 (dd, J = 12.8, 4.3 Hz, 1H), 3.36 (s, 1H), 2.62 (q, J = 7.2 Hz, 4H), 2.09 (dtd, J = 14.9, 7.4, 3.8 Hz, 2H), 1.45 (t, J = 3.5 Hz, 1H), 1.31 (s, 1H), 1.10 (t, J = 7.2 Hz, 6H). | 561.40 |
| (E)-1-((1R,5S,6s)- 6-((4-amino-7- methyl-5-(4- phenoxyphenyl)- 7H-pyrrolo[2,3- d]pyrimidin-6- yl)ethynyl)-3- azabicyclo[3.1.0] hexan-3-yl)-4-((2- methoxyethyl) (methyl)amino)but-2- en-1-one | 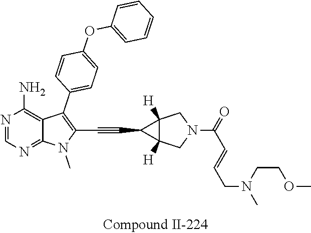 [SMILES: COCCN(C)C/C=C/C(=O)N1C[C@@H]2[C@@H](C#Cc3c(-c4ccc(Oc5ccccc5)cc4)c4c(N)ncnc4n3C)[C@@H]2C1] | ¹H NMR (400 MHz, DMSO-d₆) δ 8.18 (s, 1H), 7.53 − 7.38 (m, 4H), 7.23 − 7.08 (m, 5H), 6.59 (dt, J = 15.2, 6.3 Hz, 1H), 6.37 (d, J = 15.1 Hz, 1H), 6.22 (br, 1H), 3.85 (d, J = 10.8 Hz, 1H), 3.75 (d, J = 12.5 Hz, 1H), 3.70 (s, 3H), 3.63 (dd, J = 10.7, 4.2 Hz, 1H), 3.47 (t, J = 5.7 Hz, 2H), 3.39 (dd, J = 12.5, 4.3 Hz, 3H), 3.26 (s, 3H), 2.68 (d, J = 14.5 Hz, 2H), 2.31 (s, 3H), 2.11 (dt, J = 7.5, 3.7 Hz, 1H), 2.04 (dt, J = 7.5, 3.8 Hz, 1H), 1.48 (t, J = 3.4 Hz, 1H). | 557.4 |
| (E)-1-((1R,5S,6s)- 6-((4-amino-7- methyl-5-(4- phenoxyphenyl)- 7H-pyrrolo[2,3- d]pyrimidin-6- yl)ethynyl)-3- azabicyclo[3.1.0] hexan-3-yl)-4-(3- fluoropyrrolidin-1- yl)but-2-en-1-one | 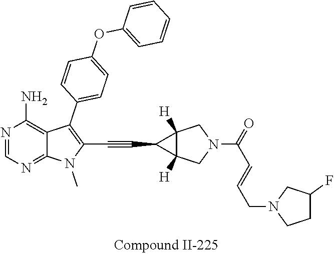 [SMILES: Cn1c(C#C[C@@H]2[C@H]3CN(C(=O)/C=C/CN4CCC(F)C4)C[C@@H]23)c(-c2ccc(Oc3ccccc3)cc2)c2c(N)ncnc21] | ¹H NMR (400 MHz, DMSO-d₆) δ 8.18 (s, 1H), 7.54 − 7.40 (m, 4H), 7.23 − 7.09 (m, 5H), 6.61 (dt, J = 15.0, 6.2 Hz, 1H), 6.33 (d, J = 15.2 Hz, 1H), 6.21 (br, 1H), 5.22 (d, J = 55.8 Hz, 1H), 3.90 − 3.82 (m, 1H), 3.80 − 3.68 (m, 4H), 3.64 (dd, J = 10.5, 4.3 Hz, 1H), 3.39 (dd, J = 12.6, 4.2 Hz, 2H), 3.29 (s, 2H), 2.84 (s, 2H), 2.74 (s, 2H), 2.43 − 2.36(m, 1H), 2.18 (dd, J = 14.3, 7.3 Hz, 1H), 2.11 (dt, J = 7.6, 3.8 Hz, 1H), 2.04 (dt, J = 7.6, 3.8 Hz, 1H), 1.99 − 1.83 (m, 1H), 1.48 (t, J = 3.5 Hz, 1H). | 577.4 |
| (E)-1-((1R,5S,6s)- 6-((4-amino-5-(2- methoxy-4-((6- methylpyridin-2- yl)oxy)phenyl)-7- methyl-7H- pyrrolo[2,3- d]pyrimidin-6- yl)ethynyl)-3- azabicyclo[3.1.0] hexan-3-yl)-4- (dimethylamino) but-2-en-1-one | 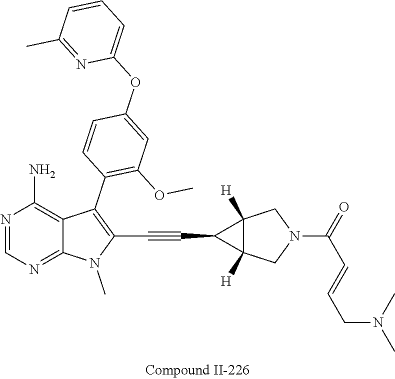 [SMILES: COc1cc(Oc2cccc(C)n2)ccc1-c1c(C#C[C@@H]2[C@H]3CN(C(=O)/C=C/CN(C)C)C[C@@H]23)n(C)c2ncnc(N)c12] | ¹H NMR (400 MHz, DMSO-d₆) δ 8.15 (s, 1H), 7.78 (t, J = 7.8 Hz, 1H), 7.29 (d, J = 8.3 Hz, 1H), 7.06 (d, J = 7.4 Hz, 1H), 6.97 (d, J = 2.3 Hz, 1H), 6.86 (d, J = 8.2 Hz, 1H), 6.78 (d, J = 8.3 Hz, 1H), 6.64 − 6.53 (m, 1H), 6.27 (d, J = 15.2 Hz, 1H), 5.96 (s, 2H), 3.84 (d, J = 10.7 Hz, 1H), 3.77 − 3.67 (m, 7H), 3.61 (dd, J = 10.8, 4.2 Hz, 1H), 3.37 (dd, J = 12.3, 4.2 Hz, 1H), 3.00 (d, J = 6.1 Hz, 2H), 2.40 (s, 3H), 2.14 (s, 6H), 2.08 − 1.95 (m, 2H), 1.45 (t, J = 3.4 Hz, 1H). | 578.3 |
| (E)-1-((1R,5S,6s)- 6-((4-amino-5-(2- methoxy-4- (pyridin-2- yloxy)phenyl)-7- methyl-7H- pyrrolo[2,3- d]pyrimidin-6- yl)ethynyl)-3- azabicyclo[3.1.0] hexan-3-yl)-4- (dimethylamino) but-2-en-1-one | 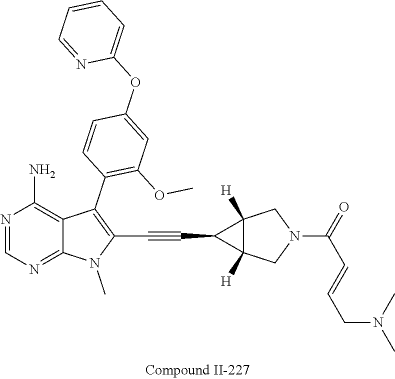 [SMILES: COc1cc(Oc2ccccn2)ccc1-c1c(C#C[C@@H]2[C@H]3CN(C(=O)/C=C/CN(C)C)C[C@@H]23)n(C)c2ncnc(N)c12] | ¹H NMR (400 MHz, Methanol-d₄) δ 8.22 − 8.11 (m, 2H), 7.91 (ddd, J = 8.3, 7.2, 2.0 Hz, 1H), 7.38 (d, J = 8.2 Hz, 1H), 7.18 (ddd, J = 7.3, 5.0, 0.9 Hz, 1H), 7.13 (d, J = 8.3 Hz, 1H), 6.94 (d, J = 2.2 Hz, 1H), 6.85 − 6.78 (m, 1H), 6.78 − 6.69 (m, 1H), 6.55 (d, J = 15.2 Hz, 1H), 3.91 (t, J = 12.4 Hz, 2H), 3.82 − 3.72 (m, 7H), 3.63 (dd, J = 7.0, 1.3 Hz, 2H), 3.53 (dd, J = 12.8, 4.2 Hz, 1H), 2.66 (s, 6H), 2.13 − 2.00 (m, 2H), 1.42 (t, J = 3.5 Hz, 1H). | 564.4 |
| (E)-1-((1R,5S,6s)- 6-((4-amino-5-(4- (2- fluorophenoxy)-2- methoxyphenyl)- 7-methyl-7H- pyrrolo[2,3- d]pyrimidin-6- yl)ethynyl)-3- azabicyclo[3.1.0] hexan-3-yl)-4- (dimethylamino) but-2-en-1-one | 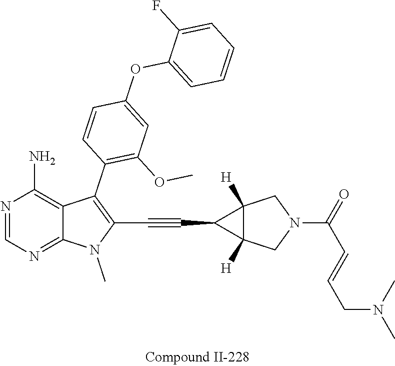 [SMILES: COc1cc(Oc2ccccc2F)ccc1-c1c(C#C[C@@H]2[C@H]3CN(C(=O)/C=C/CN(C)C)C[C@@H]23)n(C)c2ncnc(N)c12] | ¹H NMR (400 MHz, DMSO-d₆) δ 8.13 (s, 1H), 7.49 − 7.38 (m, 1H), 7.38 − 7.21 (m, 4H), 6.90 (d, J = 2.4 Hz, 1H), 6.64 − 6.50 (m, 2H), 6.27 (d, J = 15.2 Hz, 1H), 5.95 (s, 2H), 3.83 (d, J = 10.8 Hz, 1H), 3.77 − 3.70 (m, 4H), 3.68 (s, 3H), 3.61 (dd, J = 10.7, 4.2 Hz, 1H), 3.41 − 3.33 (m, 1H), 3.01 (d, J = 6.2 Hz, 2H), 2.14 (s, 6H), 2.08 − 1.90 (m, 2H), 1.43 (t, J = 3.4 Hz, 1H). | 581.2 |
| 1-((1R,5S,6s)-6- ((4-amino-7-(2- methoxyethyl)-5- (4- phenoxyphenyl)- 7H-pyrrolo[2,3- d]pyrimidin-6- yl)ethynyl)-3- azabicyclo[3.1.0] hexan-3-yl)prop-2- en-1-one | 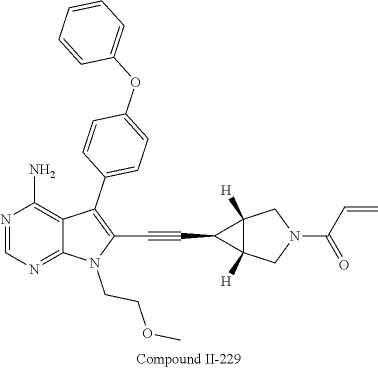 [SMILES: C=CC(=O)N1C[C@@H]2[C@@H](C#Cc3c(-c4ccc(Oc5ccccc5)cc4)c4c(N)ncnc4n3CCOC)[C@@H]2C1] | ¹H NMR (400 MHz, DMSO-d₆) δ 8.17 (s, 1H), 7.53 − 7.40 (m, 4H), 7.19 (t, J = 7.4 Hz, 1H), 7.14 (dd, J = 8.2, 4.5 Hz, 4H), 6.50 (dd, J = 16.7, 10.3 Hz, 1H), 6.38 (s, 2H), 6.10 (dd, J = 16.8, 2.4 Hz, 1H), 5.65 (dd, J = 10.3, 2.3 Hz, 1H), 4.36 (t, J = 5.9 Hz, 2H), 3.89 (d, J = 10.8 Hz, 1H), 3.79 − 3.69 (m, 3H), 3.64 (dd, J = 10.7, 4.2 Hz, 1H), 3.40 (dd, J = 12.6, 4.3 Hz, 1H), 3.27 (s, 3H), 3.18 (d, J = 5.2 Hz, 1H), 2.09 (dt, J = 7.5, 3.7 Hz, 1H), 2.02 (dt, J = 7.6, 3.9 Hz, 1H), 1.48 (t, J = 3.5 Hz, 1H) | 520.2 |
| (E)-1-((1R,5S,6s)- 6-((4-amino-7- methyl-5-(4-((6- methylpyridin-2- yl)oxy)phenyl)- 7H-pyrrolo[2,3- d]pyrimidin-6- yl)ethynyl)-3- azabicyclo[3.1.0] hexan-3-yl)-4-(3- fluoropyrrolidin-1- yl)but-2-en-1-one | 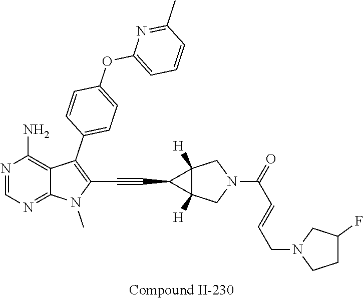 [SMILES: Cc1cccc(Oc2ccc(-c3c(C#C[C@@H]4[C@H]5CN(C(=O)/C=C/CN6CCC(F)C6)C[C@@H]45)n(C)c4ncnc(N)c34)cc2)n1] | ¹H NMR (400 MHz, DMSO-d₆) δ 8.19 (s, 1H), 7.77 (t, J = 7.7 Hz, 1H), 7.55 − 7.48 (m, 2H), 7.29 − 7.21 (m, 2H), 7.04 (d, J = 7.3 Hz, 1H), 6.86 (d, J = 8.1 Hz, 1H), 6.61 (dt, J = 15.2, 6.0 Hz, 1H), 6.30 (dt, J = 15.1, 1.7 Hz, 1H), 6.20 (s, 2H), 5.31 − 5.05 (m, 1H), 3.85 (dd, J = 10.8, 2.8 Hz, 1H), 3.75 (d, J = 12.5 Hz, 1H), 3.71 (s, 3H), 3.63 (dd, J = 10.8, 4.2 Hz, 1H), 3.38 (dd, J = 12.6, 4.3 Hz, 1H), 3.21 (dq, J = 4.7, 2.2 Hz, 2H), 2.78 (ddd, J = 16.6, 14.1, 7.7 Hz, 2H), 2.61 (ddt, J = 31.5, 11.4, 4.5 Hz, 1H), 2.37 (s, 3H), 2.36 − 2.27 (m, 1H), 2.22 − 2.00 (m, 3H), 1.87 (ddt, J = 29.1, 14.2, 6.9 Hz, 1H), 1.49 (t, J = 3.4 Hz, 1H). | 592.3 |
| (E)-1-((1R,5S,6s)- 6-((4-amino-5-(3- methoxy-4- (pyridin-2- yloxy)phenyl)-7- methyl-7H- pyrrolo[2,3- d]pyrimidin-6- yl)ethynyl)-3- azabicyclo[3.1.0] hexan-3-yl)-4-((2- methoxyethyl) (methyl)amino) but-2- en-1-one | 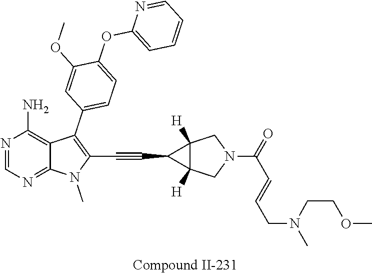 [SMILES: COCCN(C)C/C=C/C(=O)N1C[C@@H]2[C@@H](C#Cc3c(-c4ccc(Oc5ccccn5)c(OC)c4)c4c(N)ncnc4n3C)[C@@H]2C1] | ¹H NMR (400 MHz, Methanol-d₄) δ 8.19 (s, 1H), 8.06 (dd, J = 5.2, 1.9 Hz, 1H), 7.91 − 7.82 (m, 1H), 7.29 − 7.22 (m, 2H), 7.20 − 7.04 (m, 3H), 6.82 (dt, J = 15.3, 6.2 Hz, 1H), 6.42 (d, J = 15.2 Hz, 1H), 3.93 (dd, J = 18.4, 11.8 Hz, 2H), 3.83 − 3.70 (m, 7H), 3.53 (q, J = 6.8, 6.0 Hz, 3H), 3.36 (s, 3H), 3.28 (d, J = 6.3 Hz, 2H), 2.64 (t, J = 5.5 Hz, 2H), 2.32 (s, 3H), 2.11 (dtd, J = 14.9, 7.3, 3.7 Hz, 2H), 1.48 (t, J = 3.4 Hz, 1H). | 608.4 |
| (E)-1-((1R,5S,6s)- 6-((4-amino-5-(4- (3- fluorophenoxy) phenyl)-7-methyl-7H- pyrrolo[2,3- d]pyrimidin-6- yl)ethynyl)-3- azabicyclo[3.1.0] hexan-3-yl)-4-((2- methoxyethyl) (methyl)amino)but-2- en-1-one | 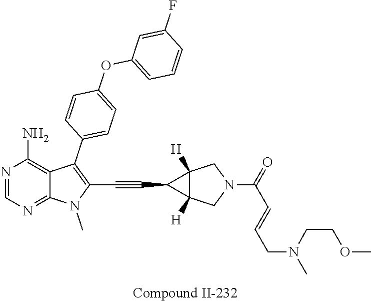 [SMILES: COCCN(C)C/C=C/C(=O)N1C[C@@H]2[C@@H](C#Cc3c(-c4ccc(Oc5cccc(F)c5)cc4)c4c(N)ncnc4n3C)[C@@H]2C1] | ¹H NMR (400 MHz, DMSO-d₆) δ 8.18 (s, 1H), 7.55 − 7.47 (m, 2H), 7.45 (q, J = 7.8 Hz, 1H), 7.24 − 7.16 (m, 2H), 7.06 − 6.94 (m, 3H), 6.59 (dt, J = 15.2, 6.0 Hz, 1H), 6.40 (s, 2H), 6.30 (d, J = 15.2 Hz, 1H), 3.84 (d, J = 10.7 Hz, 1H), 3.74 (d, J = 12.5 Hz, 1H), 3.70 (s, 3H), 3.62 (dd, J = 10.7, 4.3 Hz, 1H), 3.45 − 3.35 (m, 3H), 3.24 (s, 3H), 3.16 − 3.10 (m, 2H), 2.47 (d, J = 5.9 Hz, 2H), 2.18 (s, 3H), 2.11 (dt, J = 7.4, 3.7 Hz, 1H), 2.03 (dt, J = 7.5, 3.8 Hz, 1H), 1.48 (t, J = 3.4 Hz, 1H). | 595.4 |
| (E)-1-((1R,5S,6s)- 6-((4-amino-5-(3- methoxy-4- (pyridin-2- yloxy)phenyl)-7- methyl-7H- pyrrolo[2,3- d]pyrimidin-6- yl)ethynyl)-3- azabicyclo[3.1.0] hexan-3-yl)-4- (dimethylamino) but-2-en-1-one | 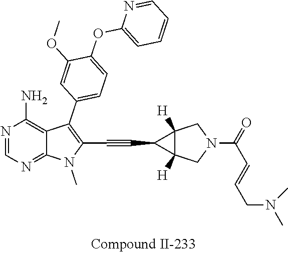 [SMILES: COc1cc(-c2c(C#C[C@@H]3[C@H]4CN(C(=O)/C=C/CN(C)C)C[C@@H]34)n(C)c3ncnc(N)c23)ccc1Oc1ccccn1] | ¹H NMR (400 MHz, DMSO-d₆) δ 8.20 (s, 1H), 8.16 − 8.10 (m, 1H), 7.83 (t, J = 7.6 Hz, 1H), 7.26 (d, J = 7.5 Hz, 2H), 7.09 (d, J = 7.1 Hz, 2H), 7.04 (d, J = 8.3 Hz, 1H), 6.59 (dt, J = 13.5, 6.2 Hz, 1H), 6.28 (d, J = 15.3 Hz, 2H), 3.86 (d, J = 10.7 Hz, 1H), 3.74 (t, J = 8.8 Hz, 7H), 3.67 − 3.60 (m, 1H), 3.39 (dd, J = 12.8, 4.3 Hz, 2H), 3.01 (d, J = 6.2 Hz, 2H), 2.14 (s, 7H), 2.09 − 2.03 (m, 1H), 1.53 (d, J = 3.6 Hz, 1H). | 564.2 |
| (E)-1-((1R,5S,6s)- 6-((4-amino-5-(4- (3- fluorophenoxy) phenyl)-7- methyl-7H- pyrrolo[2,3- d]pyrimidin-6- yl)ethynyl)-3- azabicyclo[3.1.0] hexan-3-yl)-4-(3- fluoropyrrolidin-1- yl)but-2-en-1-one | 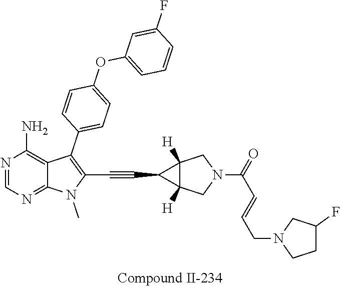 [SMILES: Cn1c(C#C[C@@H]2[C@H]3CN(C(=O)/C=C/CN4CCC(F)C4)C[C@@H]23)c(-c2ccc(Oc3cccc(F)c3)cc2)c2c(N)ncnc21] | ¹H NMR (400 MHz, DMSO-d₆) δ 8.18 (s, 1H), 7.56 − 7.47 (m, 2H), 7.51 − 7.40 (m, 1H), 7.24 − 7.16 (m, 2H), 7.06 − 6.94 (m, 3H), 6.62 (dt, J = 15.2, 6.0 Hz, 1H), 6.30 (dt, J = 15.3, 1.6 Hz, 1H), 6.23 (s, 1H), 5.33 − 5.08 (m, 1H), 3.85 (dd, J = 10.7, 2.7 Hz, 1H), 3.75 (d, J = 12.5 Hz, 1H), 3.71 (s, 3H), 3.69 − 3.59 (m, 1H), 3.38 (dd, J = 12.6, 4.3 Hz, 1H), 3.24 − 3.18 (m, 2H), 2.85 − 2.72 (m, 2H), 2.70 − 2.54 (m, 1H), 2.33 (s, 1H), 2.22 − 2.02 (m, 2H), 2.06 − 1.99 (m, 1H), 1.87 (ddt, J = 29.3, 14.4, 7.2 Hz, 1H), 1.48 (t, J = 3.4 Hz, 1H). | 595.3 |
| (E)-1-((1R,5S,6s)- 6-((4-amino-5-(2- methoxy-4-((6- methylpyridin-2- yl)oxy)phenyl)-7- methyl-7H- pyrrolo[2,3- d]pyrimidin-6- yl)ethynyl)-3- azabicyclo[3.1.0] hexan-3-yl)-4-((2- methoxyethyl) (methyl)amino)but-2- en-1-one | 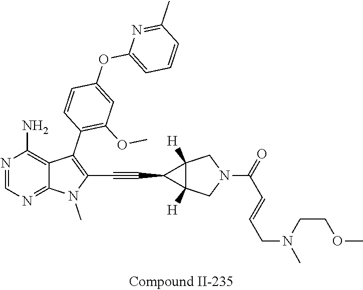 [SMILES: COCCN(C)C/C=C/C(=O)N1C[C@@H]2[C@@H](C#Cc3c(-c4ccc(Oc5cccc(C)n5)cc4OC)c4c(N)ncnc4n3C)[C@@H]2C1] | ¹H NMR (400 MHz, Methanol-d₄) δ 8.15 (s, 1H), 7.77 (t, J = 7.8 Hz, 1H), 7.37 (d, J = 8.2 Hz, 1H), 7.06 (d, J = 7.4 Hz, 1H), 6.95 (d, J = 2.2 Hz, 1H), 6.89 − 6.76 (m, 3H), 6.41 (d, J = 15.3 Hz, 1H), 3.91 (dd, J = 17.8, 11.8 Hz, 2H), 3.79 (d, J = 1.1 Hz, 6H), 3.72 (dd, J = 10.9, 4.2 Hz, 1H), 3.57 − 3.46 (m, 3H), 3.36 (s, 3H), 3.32 − 3.25 (m, 2H), 2.65 (t, J = 5.5 Hz, 2H), 2.47 (s, 3H), 2.33 (s, 3H), 2.05 (dtt, J = 16.9, 11.2, 5.7 Hz, 2H), 1.42 (t, J = 3.4 Hz, 1H). | 622.4 |
| (E)-1-((1R,5S,6s)- 6-((4-amino-5-(2- methoxy-4-((6- methylpyridin-2- yl)oxy)phenyl)-7- methyl-7H- pyrrolo[2,3- d]pyrimidin-6- yl)ethynyl)-3- azabicyclo[3.1.0] hexan-3-yl)-4-(3- fluoropyrrolidin-1- yl)but-2-en-1-one | 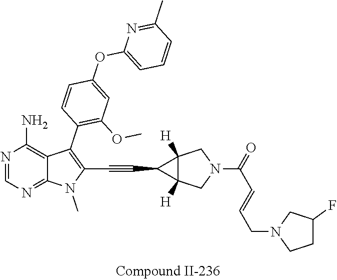 [SMILES: COc1cc(Oc2cccc(C)n2)ccc1-c1c(C#C[C@@H]2[C@H]3CN(C(=O)/C=C/CN4CCC(F)C4)C[C@@H]23)n(C)c2ncnc(N)c12] | ¹H NMR (400 MHz, DMSO-d₆) δ 8.14 (s, 1H), 7.77 (t, J = 7.7 Hz, 1H), 7.29 (d, J = 8.3 Hz, 1H), 7.06 (d, J = 7.3 Hz, 1H), 6.96 (d, J = 2.3 Hz, 1H), 6.86 (d, J = 8.1 Hz, 1H), 6.78 (dd, J = 8.3, 2.2 Hz, 1H), 6.61 (dt, J = 15.2, 6.0 Hz, 1H), 6.29 (dt, J = 15.1, 1.6 Hz, 1H), 5.96 (s, 2H), 5.30 − 5.07 (m, 1H), 3.84 (d, J = 10.9 Hz, 1H), 3.71 (d, J = 17.2 Hz, 7H), 3.61 (dd, J = 11.0, 4.0 Hz, 1H), 3.38 (d, J = 4.3 Hz, 1H), 3.24 − 3.18 (m, 2H), 2.79 (td, J = 14.6, 13.1, 9.9 Hz, 2H), 2.70 − 2.54 (m, 1H), 2.39 (s, 3H), 2.33 (d, J = 8.4 Hz, 1H), 2.22 − 2.04 (m, 1H), 2.07 − 1.97 (m, 1H), 2.00 − 1.77 (m, 1H), 1.45 (t, J = 3.4 Hz, 1H). | 622.4 |
| (E)-1-((1R,5S,6s)- 6-((4-amino-5-(3- methoxy-4-((6- methylpyridin-2- yl)oxy)phenyl)-7- methyl-7H- pyrrolo[2,3- d]pyrimidin-6- yl)ethynyl)-3- azabicyclo[3.1.0] hexan-3-yl)-4-((2- methoxyethyl) (methyl)amino)but-2- en-1-one | 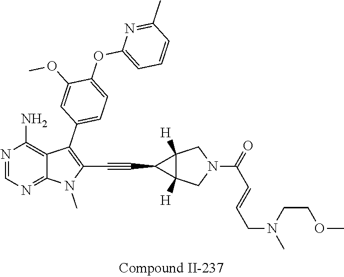 [SMILES: COCCN(C)C/C=C/C(=O)N1C[C@@H]2[C@@H](C#Cc3c(-c4ccc(Oc5cccc(C)n5)c(OC)c4)c4c(N)ncnc4n3C)[C@@H]2C1] | ¹H NMR (400 MHz, DMSO-d₆) δ 8.20 (s, 1H), 7.69 (dd, J = 8.3, 7.3 Hz, 1H), 7.28 − 7.21 (m, 2H), 7.09 (dd, J = 8.1, 2.0 Hz, 1H), 6.96 (d, J = 7.3 Hz, 1H), 6.74 (d, J = 8.2 Hz, 1H), 6.64 − 6.50 (m, 1H), 6.30 (dt, J = 15.1, 1.6 Hz, 1H), 3.85 (d, J = 10.7 Hz, 1H), 3.74 (d, J = 14.2 Hz, 7H), 3.63 (dd, J = 10.7, 4.2 Hz, 1H), 3.45 − 3.34 (m, 3H), 3.23 (s, 3H), 3.13 (dd, J = 6.1, 1.5 Hz, 2H), 2.47 (d, J = 5.9 Hz, 2H), 2.33 (s, 3H), 2.18 (s, 3H), 2.12 (dt, J = 7.5, 3.7 Hz, 1H), 2.04 (dt, J = 7.5, 3.8 Hz, 1H), 1.53 (t, J = 3.4 Hz, 1H). | 622.4 |
| (E)-1-((1R,5S,6s)- 6-((4-amino-5-(3- methoxy-4-((6- methylpyridin-2- yl)oxy)phenyl)-7- methyl-7H- pyrrolo[2,3- d]pyrimidin-6- yl)ethynyl)-3- azabicyclo[3.1.0] hexan-3-yl)-4-(3- fluoropyrrolidin-1- yl)but-2-en-1-one | 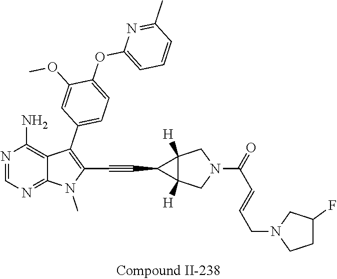 [SMILES: COc1cc(-c2c(C#C[C@@H]3[C@H]4CN(C(=O)/C=C/CN5CCC(F)C5)C[C@@H]34)n(C)c3ncnc(N)c23)ccc1Oc1cccc(C)n1] | ¹H NMR (400 MHz, DMSO-d₆) δ 8.20 (s, 1H), 7.69 (t, J = 7.8 Hz, 1H), 7.28 − 7.21 (m, 2H), 7.09 (dd, J = 8.1, 2.0 Hz, 1H), 6.96 (d, J = 7.3 Hz, 1H), 6.74 (d, J = 8.2 Hz, 1H), 6.62 (dt, J = 15.2, 6.0 Hz, 1H), 6.30 (d, J = 15.2 Hz, 1H), 5.19 (d, J = 56.0 Hz, 1H), 3.85 (dd, J = 10.8, 2.8 Hz, 1H), 3.74 (dd, J = 13.4, 3.8 Hz, 7H), 3.67 − 3.60 (m, 1H), 3.38 (dd, J = 12.4, 4.3 Hz, 1H), 3.25 − 3.18 (m, 2H), 2.86 − 2.75 (m, 2H), 2.69 − 2.54 (m, 1H), 2.33 (s, 4H), 2.22 − 2.00 (m, 3H), 1.87 (ddt, J = 29.6, 14.4, 7.1 Hz, 1H), 1.53 (t, J = 3.4 Hz, 1H). | 622.3 |
| (E)-1-((1R,5S,6s)- 6-((4-amino-5-(3- methoxy-4-((6- methylpyridin-2- yl)oxy)phenyl)-7- methyl-7H- pyrrolo[2,3- d]pyrimidin-6- yl)ethynyl)-3- azabicyclo[3.1.0] hexan-3-yl)-4- (dimethylamino) but-2-en-1-one | 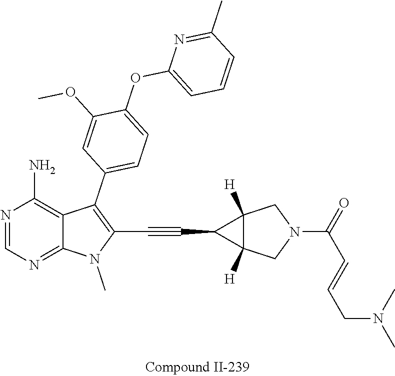 [SMILES: COc1cc(-c2c(C#C[C@@H]3[C@H]4CN(C(=O)/C=C/CN(C)C)C[C@@H]34)n(C)c3ncnc(N)c23)ccc1Oc1cccc(C)n1] | ¹H NMR (400 MHz, DMSO-d₆) δ 8.20 (s, 1H), 7.69 (t, J = 7.8 Hz, 1H), 7.28 − 7.21 (m, 2H), 7.09 (dd, J = 8.1, 2.0 Hz, 1H), 6.96 (d, J = 7.3 Hz, 1H), 6.74 (d, J = 8.2 Hz, 1H), 6.59 (dt, J = 15.2, 6.2 Hz, 1H), 6.28 (dt, J = 15.1, 1.6 Hz, 1H), 3.85 (d, J = 10.7 Hz, 1H), 3.74 (dd, J = 13.5, 3.1 Hz, 7H), 3.63 (dd, J = 10.7, 4.3 Hz, 1H), 3.38 (dd, J = 12.5, 4.3 Hz, 1H), 3.04 − 2.98 (m, 2H), 2.33 (s, 3H), 2.14 (s, 6H), 2.11 (q, J = 3.7 Hz, 1H), 2.04 (dt, J = 7.3, 3.8 Hz, 1H), 1.53 (t, J = 3.4 Hz, 1H). | 578.3 |
| (E)-1-((1R,5S,6s)- 6-((4-amino-7- methyl-5-(4-((4- methylpyridin-2- yl)oxy)phenyl)- 7H-pyrrolo[2,3- d]pyrimidin-6- yl)ethynyl)-3- azabicyclo[3.1.0] hexan-3-yl)-4-(3- fluoropyrrolidin-1- yl)but-2-en-1-one | 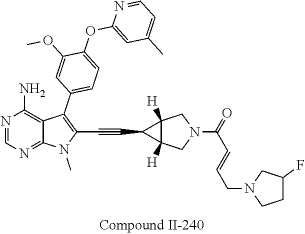 [SMILES: COc1cc(-c2c(C#C[C@@H]3[C@H]4CN(C(=O)/C=C/CN5CCC(F)C5)C[C@@H]34)n(C)c3ncnc(N)c23)ccc1Oc1cc(C)ccn1] | ¹H NMR (400 MHz, DMSO-d₆) δ 8.19 (s, 1H), 8.07 (d, J = 5.1 Hz, 1H), 7.55 − 7.47 (m, 2H), 7.29 − 7.21 (m, 2H), 7.02 (d, J = 5.2 Hz, 1H), 6.95 (s, 1H), 6.61 (dt, J = 15.2, 6.0 Hz, 1H), 6.30 (d, J = 15.3 Hz, 1H), 6.18 (s, 1H), 5.19 (dt, J = 55.6, 5.8 Hz, 1H), 3.86 (dd, J = 10.9, 2.8 Hz, 1H), 3.75 (d, J = 12.6 Hz, 1H), 3.71 (s, 3H), 3.67 − 3.59 (m, 1H), 3.38 (dd, J = 12.6, 4.3 Hz, 1H), 3.24 (d, J = 10.8 Hz, 2H), 2.79 (td, J = 14.6, 12.9, 9.5 Hz, 2H), 2.69 − 2.54 (m, 1H), 2.36 (s, 4H), 2.22 − 2.01 (m, 3H), 1.87 (ddt, J = 28.7, 13.6, | 592.3 |
| | | 6.7 Hz, 1H), 1.49 (t, J = 3.4 Hz, 1H). | |
| (E)-1-((1R,5S,6s)- 6-((4-amino-7- methyl-5-(4-((4- methylpyridin-2- yl)oxy)phenyl)- 7H-pyrrolo[2,3- d]pyrimidin-6- yl)ethynyl)-3- azabicyclo[3.1.0] hexan-3-yl)-4- (dimethylamino) but-2-en-1-one | 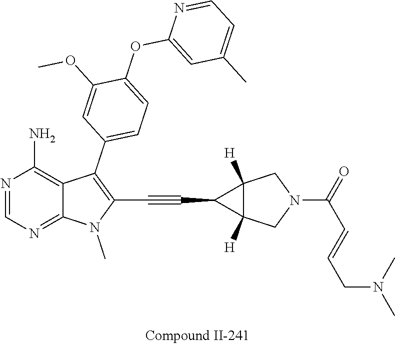 [SMILES: COc1cc(-c2c(C#C[C@@H]3[C@H]4CN(C(=O)/C=C/CN(C)C)C[C@@H]34)n(C)c3ncnc(N)c23)ccc1Oc1cc(C)ccn1] | ¹H NMR (400 MHz, DMSO-d₆) δ 8.19 (s, 1H), 8.07 (d, J = 5.1 Hz, 1H), 7.52 (d, J = 8.2 Hz, 2H), 7.25 (d, J = 8.2 Hz, 2H), 7.02 (d, J = 5.1 Hz, 1H), 6.95 (s, 1H), 6.59 (dt, J = 14.7, 6.0 Hz, 1H), 6.28 (d, J = 15.2 Hz, 1H), 6.17 (s, 1H), 3.86 (d, J = 10.7 Hz, 1H), 3.79 − 3.70 (m, 4H), 3.63 (dd, J = 10.6, 4.2 Hz, 1H), 3.39 (dd, J = 12.6, 4.4 Hz, 1H), 3.01 (d, J = 6.2 Hz, 2H), 2.37 (s, 3H), 2.15 (s, 7H), 2.06 (d, J = 5.0 Hz, 1H), 1.52 − 1.46 (m, 1H). | 548.2 |
| 1-((2S)-4-(3-((4- amino-5-(3- methoxy-4-(6- methylpyridin-2- yloxy)phenyl)-7- methyl-7H- pyrrolo[2,3- d]pyrimidin-6- yl)ethynyl)azetidin- 1-yl)-2- (hydroxymethyl) piperidin-1-yl)prop- 2-en-1-one | 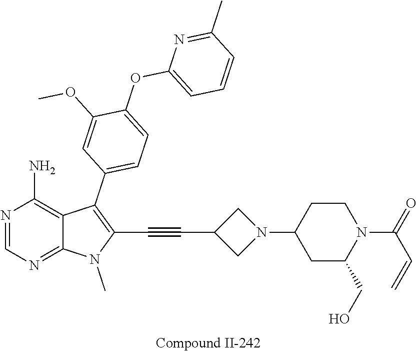 [SMILES: C=CC(=O)N1CCC(N2CC(C#Cc3c(-c4ccc(Oc5cccc(C)n5)c(OC)c4)c4c(N)ncnc4n3C)C2)C[C@H]1CO] | ¹H NMR (400 MHz, DMSO-d₆) δ 8.20 (s, 1H), 7.68 (t, J = 7.7 Hz, 1H), 7.30 − 7.18 (m, 2H), 7.12 (d, J = 8.2 Hz, 1H), 6.95 (d, J = 7.3 Hz, 1H), 6.88 − 6.65 (m, 2H), 6.33 (br, 1H), 6.04 (dd, J = 16.8, 2.6 Hz, 1H), 5.59 (d, J = 10.6 Hz, 1H), 4.86 (s, 1H), 4.29 − 3.91 (m, 2H), 3.79 (s, 6H), 3.58 (d, J = 7.1 Hz, 2H), 3.52 − 3.37 (m, 2H), 3.08 − 2.92 (m, 2H), 2.36 (d, J = 27.1 Hz, 4H), 1.53 (dd, J = 30.7, 18.7 Hz, 3H), 1.36 (s, 1H). | 608.25 |
| 1-(4-[3-[2-(4- amino-5-[3- methoxy-4-[(6- methylpyridin-2- yl)oxy]phenyl]-7- methyl-7H- pyrrolo[2,3- d]pyrimidin-6- yl)ethynyl]-3- fluoroazetidin-1- yllpiperidin-1- yl)prop-2-en-1-one | 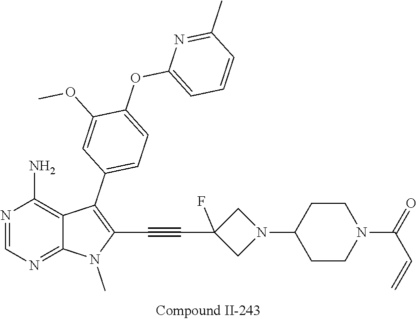 [SMILES: C=CC(=O)N1CCC(N2CC(F)(C#Cc3c(-c4ccc(Oc5cccc(C)n5)c(OC)c4)c4c(N)ncnc4n3C)C2)CC1] | ¹H NMR (400 MHz, DMSO-d₆) δ 8.24 (s, 1H), 7.68 (t, J = 7.7 Hz, 1H), 7.34 − 7.22 (m, 2H), 7.13 (d, J = 8.2 Hz, 1H), 6.95 (d, J = 7.3 Hz, 1H), 6.86 − 6.69 (m, 2H), 6.65 − 6.15 (br, 1H), 6.06 (dd, J = 16.8, 2.5 Hz, OH), 5.64 (dd, J = 10.4, 2.5 Hz, 1H), 4.04 (d, J = 12.9 Hz, 1H), 3.84 (d, J = 13.6 Hz, 1H), 3.77 (s, 3H), 3.75 (s, 3H), 3.64 (dd, J = 15.9, 8.7 Hz, 2H), 3.54 (dd, J = 20.1, 8.7 Hz, 2H), 3.13 (t, J = 11.8 Hz, 1H), 2.91 (t, J = 11.4 Hz, 1H), 2.39 (m, J = 9.5 Hz, 1H), 2.32 (s, 3H), 1.63 (s, 1H), 1.08 (s, 1H). | 596.30 |
| 1-((2S)-4-(3-((4- amino-7-methyl-5- (4-(pyridin-2- yloxy)phenyl)-7H- pyrrolo[2,3- d]pyrimidin-6- yl)ethynyl)azetidin- 1-yl)-2- (hydroxymethyl) piperidin-1-yl)prop- 2-en-1-one | 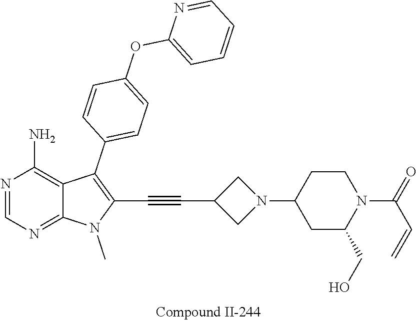 [SMILES: C=CC(=O)N1CCC(N2CC(C#Cc3c(-c4ccc(Oc5ccccn5)cc4)c4c(N)ncnc4n3C)C2)C[C@H]1CO] | ¹H NMR (400 MHz, DMSO-d₆) δ 8.22 (m, 1H), 7.91 (ddd, J = 8.6, 7.2, 2.0 Hz, 1H), 7.60 − 7.52 (m, 2H), 7.31 − 7.23 (m, 2H), 7.18 (dd, J = 7.2, 4.9 Hz, 1H), 7.10 (d, J = 8.3 Hz, 1H), 6.77 (dd, J = 16.8, 10.5 Hz, 1H), 6.25 (s, 2H), 6.04 (dd, J = 16.8, 2.5 Hz, 1H), 5.60 (d, J = 11.0 Hz, 1H), 4.89 (s, 1H), 4.17 − 3.99 (d, 2H), 3.74 (s, 3H), 3.56 (t, J = 7.1 Hz, 3H), 3.42 (p, J = 7.1 Hz, 1H), 3.08 − 2.86 (m, 3H), 2.44 − 2.37 (m, 1H), 1.60 (s, 1H), 1.50 (d, J = 14.7 Hz, 2H), 1.36 (s, 1H). | 564.3 |
| 1-((2S)-4-(3-((4- amino-5-(2- methoxy-4- (pyridin-2- yloxy)phenyl)-7- methyl-7H- pyrrolo[2,3- d]pyrimidin-6- yl)ethynyl)azetidin- 1-yl)-2- (hydroxymethyl) piperidin-1-yl)prop- 2-en-1-one | 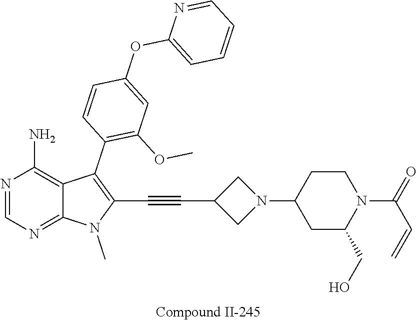 [SMILES: C=CC(=O)N1CCC(N2CC(C#Cc3c(-c4ccc(Oc5ccccn5)cc4OC)c4c(N)ncnc4n3C)C2)C[C@H]1CO] | ¹H NMR (400 MHz, DMSO-d₆) δ 8.23 (dd, J = 5.0, 1.9 Hz, 1H), 8.15 (s, 1H), 7.89 (ddd, J = 8.8, 7.2, 2.1 Hz, 1H), 7.33 (d, J = 8.3 Hz, 1H), 7.19 (dd, J = 7.2, 4.9 Hz, 1H), 7.10 (d, J = 8.3 Hz, 1H), 6.97 (d, J = 2.2 Hz, 1H), 6.84 − 6.71 (m, 2H), 6.15 (s, 2H), 6.04 (dd, J = 16.7, 2.5 Hz, 1H), 5.60 (d, J = 10.4 Hz, 1H), 4.88 (s, 1H), 3.98 (s, 2H), 3.73 (d, J = 4.3 Hz, 6H), 3.53 (d, J = 8.0 Hz, 3H), 3.40 − 3.32 (m, 2H), 2.96 (ddd, J = 20.2, 13.0, 7.3 Hz, 3H), 2.41 − 2.35 (m, 1H), 1.58 (s, 1H), 1.55 − 1.45 (m, 2H), 1.35 (s, 1H). | 594.3 |
| 1-(4-(3-((4-amino- 5-(3-methoxy-4- phenoxyphenyl)-7- methyl-7H- pyrrolo[2,3- d]pyrimidin-6- yl)ethynyl)-3- fluoroazetidin-1- yl)piperidin-1- yl)prop-2-en-1-one | 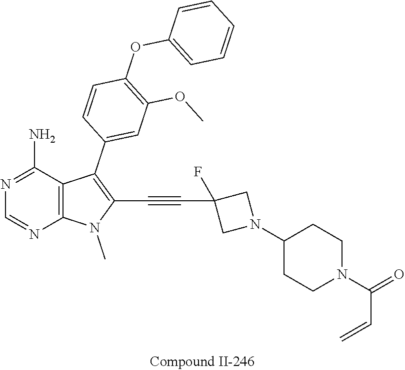 [SMILES: C=CC(=O)N1CCC(N2CC(F)(C#Cc3c(-c4ccc(Oc5ccccc5)c(OC)c4)c4c(N)ncnc4n3C)C2)CC1] | ¹H NMR (400 MHz, Methanol-d₄) δ 8.24 (s, 1H), 7.36 − 7.32 (m, 3H), 7.16 (q, J = 8.2 Hz, 2H), 7.08 (t, J = 7.4 Hz, 1H), 6.97 (d, J = 8.0 Hz, 2H), 6.75 (dd, J = 16.8, 10.6 Hz, 1H), 6.18 (d, J = 16.8 Hz, 1H), 5.73 (dd, J = 10.7, 1.8 Hz, 1H), 4.31 (d, J = 13.2 Hz, 1H), 4.00 (d, J = 14.0 Hz, 1H), 3.87 (s, 6H), 3.73 (dd, J = 15.4, 8.9 Hz, 2H), 3.63 (dd, J = 19.4, 8.9 Hz, 2H), 3.21 (t, J = 12.2 Hz, 1H), 2.97 (t, J = 12.1 Hz, 1H), 2.50 (dd, J = 9.2, 4.8 Hz, 1H), 1.79 (d, J = 12.9 Hz, 2H), 1.24 (s, 2H). | 581.25 |
| 1-((3R,4S)-4-(3- ((4-amino-5-(3- methoxy-4- phenoxyphenyl)-7- methyl-7H- pyrrolo[2,3- d]pyrimidin-6- yl)ethynyl)azetidin- 1-yl)-3- hydroxypiperidin- 1-yl)prop-2-en-1- one | 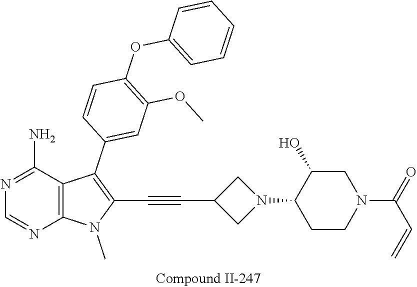 [SMILES: C=CC(=O)N1CC[C@H](N2CC(C#Cc3c(-c4ccc(Oc5ccccc5)c(OC)c4)c4c(N)ncnc4n3C)C2)[C@H](O)C1] | ¹H NMR (400 MHz, DMSO-d₆) δ 8.20 (s, 1H), 7.39 − 7.30 (m, 2H), 7.27 (d, J = 1.9 Hz, 1H), 7.18 − 7.02 (m, 3H), 7.00 − 6.93 (m, 2H), 6.74 (td, J = 17.7, 10.5 Hz, 1H), 6.04 (d, J = 16.4 Hz, 1H), 5.62 (d, J = 10.3 Hz, 1H), 4.39 (s, 1H), 4.00 (d, J = 13.0 Hz, 1H), 3.80 (s, 3H), 3.74 (s, 3H), 3.68 (s, 1H), 3.58 (s, 3H), 3.52 − 3.44 (m, 1H), 3.16 (d, J = 13.7 Hz, 1H), 3.08 (s, 2H), 2.85 (s, 1H), 2.27 (s, 1H), 1.36 (s, 2H). | 579.3 |
| 1-((2S)-4-(3-((4- amino-5-(3- methoxy-4- phenoxyphenyl)-7- methyl-7H- pyrrolo[2,3- d]pyrimidin-6- yl)ethynyl)azetidin- 1-yl)-2- (hydroxymethyl) piperidin-1-yl)prop- 2-en-1-one | 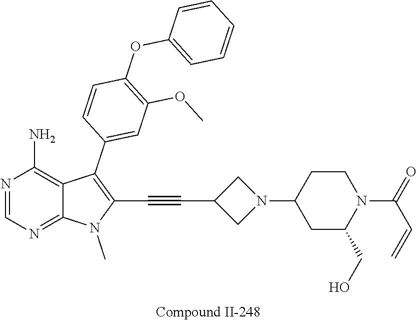 [SMILES: C=CC(=O)N1CCC(N2CC(C#Cc3c(-c4ccc(Oc5ccccc5)c(OC)c4)c4c(N)ncnc4n3C)C2)C[C@H]1CO] | ¹H NMR (400 MHz, Methanol-d₄) δ 8.21 (s, 1H), 7.37 − 7.28 (m, 3H), 7.17 (d, J = 8.4 Hz, 1H), 7.13 (d, J = 8.2 Hz, 1H), 7.07 (t, J = 7.4 Hz, 1H), 6.97 (d, J = 8.0 Hz, 2H), 6.79 (s, 1H), 6.17 (d, J = 16.7 Hz, 1H), 5.71 (d, J = 10.6 Hz, 1H), 4.12 (s, 2H), 3.85 (d, J = 12.7 Hz, 7H), 3.67 (s, 4H), 3.47 (d, J = 6.9 Hz, 1H), 3.16 (s, 3H), 2.51 (s, 1H), 1.75 (s, 2H), 1.63 (s, 2H). | 593.30 |
| 1-((3R,4S)-4-(3- ((4-amino-5-(4-(3- fluorophenoxy) phenyl)-7-methyl-7H- pyrrolo[2,3- d]pyrimidin-6- yl)ethynyl)azetidin- 1-yl)-3- hydroxypiperidin- 1-yl)prop-2-en-1- one | 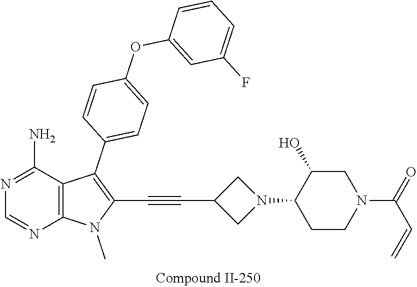 [SMILES: C=CC(=O)N1CC[C@H](N2CC(C#Cc3c(-c4ccc(Oc5cccc(F)c5)cc4)c4c(N)ncnc4n3C)C2)[C@H](O)C1] | ¹H NMR (400 MHz, DMSO-d₆) δ 8.19 (s, 1H), 7.55 (d, J = 8.2 Hz, 2H), 7.46 (q, J = 8.1 Hz, 1H), 7.20 (d, J = 8.1 Hz, 2H), 7.04 − 6.91 (m, 3H), 6.74 (td, J = 16.7, 10.4 Hz, 1H), 6.26 (s, 1H), 6.04 (dd, J = 16.8, 2.5 Hz, 1H), 5.67 − 5.58 (m, 1H), 4.45 (dd, J = 49.1, 3.4 Hz, 1H), 4.01 (d, J = 12.9 Hz, 1H), 3.79 (dd, J = 13.6, 4.9 Hz, 1H), 3.66 (d, J = 6.2 Hz, 1H), 3.57 (d, J = 6.9 Hz, 2H), 3.44 (p, J = 7.6 Hz, 1H), 3.28 − 3.03 (m, 3H), 2.86 (dt, J = 13.8, 6.6 Hz, 1H), 2.41 − 2.22 (m, 1H), 1.50 − 1.36 (m, 2H). | 567.30 |
| 1-((2S)-4-(3-((4- amino-5-(4-(3- fluorophenoxy) phenyl)-7-methyl-7H- pyrrolo[2,3- d]pyrimidin-6- yl)ethynyl)azetidin- 1-yl)-2- (hydroxymethyl) piperidin-1-yl)prop- 2-en-1-one | 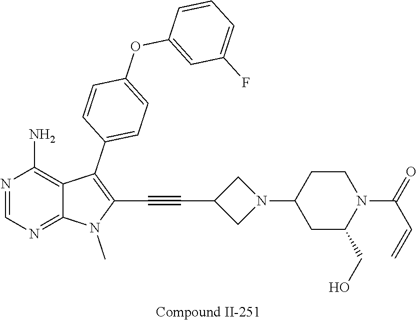 [SMILES: C=CC(=O)N1CCC(N2CC(C#Cc3c(-c4ccc(Oc5cccc(F)c5)cc4)c4c(N)ncnc4n3C)C2)C[C@H]1CO] | ¹H NMR (400 MHz, Methanol-d₄) δ 8.19 (d, J = 1.9 Hz, 1H), 7.61 − 7.58 (m, 2H), 7.45 (q, J = 7.8 Hz, 1H), 7.19 (dd, J = 8.6, 2.1 Hz, 2H), 6.96 − 6.87 (m, 2H), 6.87 − 6.72 (m, 2H), 6.17 (d, J = 16.8 Hz, 1H), 5.71 (d, J = 10.6 Hz, 1H), 4.24 (d, J = 60.5 Hz, 2H), 3.82 (d, J = 2.0 Hz, 4H), 3.64 (t, J = 7.0 Hz, 3H), 3.45 (q, J = 7.4 Hz, 1H), 3.16 (s, 3H), 2.51 (s, 1H), 1.75 (s, 2H), 1.66 − 1.58 (m, 2H). | 581.25 |
| 1-(4-(3-((4-amino- 5-(4-chloro-3- methoxyphenyl)- 7-methyl-7H- pyrrolo[2,3- d]pyrimidin-6- yl)ethynyl)azetidin- 1-yl)piperidin-1- yl)prop-2-en-1-one | 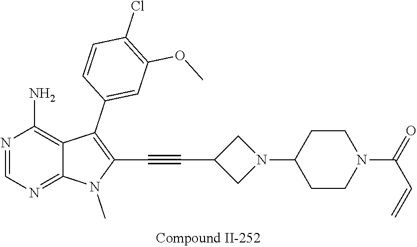 [SMILES: C=CC(=O)N1CCC(N2CC(C#Cc3c(-c4ccc(Cl)c(OC)c4)c4c(N)ncnc4n3C)C2)CC1] | ¹H NMR (400 MHz, DMSO-d₆) δ 8.20 (s, 1H), 7.54 (d, J = 8.1 Hz, 1H), 7.22 (d, J = 2.0 Hz, 1H), 7.09 (dd, J = 8.1, 1.9 Hz, 1H), 6.80 (dd, J = 16.7, 10.5 Hz, 1H), 6.30 (s, 2H), 6.08 (dd, J = 16.6, 2.4 Hz, 1H), 5.65 (dd, J = 10.5, 2.4 Hz, 1H), 4.05 (s, 1H), 3.90 (s, 3H), 3.86 (s, 1H), 3.73 (s, 3H), 3.63 (s, 2H), 3.52 (s, 1H), 3.16 (s, 4H), 2.94 (s, 1H), 2.34 (s, 1H), 1.66 (s, 2H), 1.09 (s, 2H). | 505.3 |
| 1-(4-(3-((4-amino- 5-(4-chloro-3- methoxyphenyl)- 7-methyl-7H- pyrrolo[2,3- d]pyrimidin-6- yl)ethynyl)-3- fluoroazetidin-1- yl)piperidin-1- yl)prop-2-en-1-one | 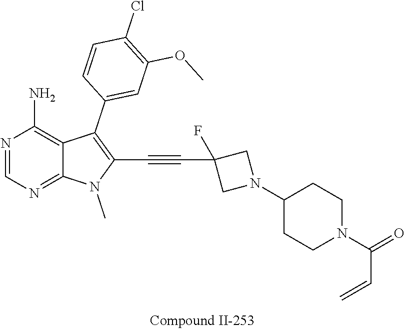 [SMILES: C=CC(=O)N1CCC(N2CC(F)(C#Cc3c(-c4ccc(Cl)c(OC)c4)c4c(N)ncnc4n3C)C2)CC1] | ¹H NMR (400 MHz, Methanol-d₄) δ 8.26 (s, 1H), 7.53 (d, J = 8.1 Hz, 1H), 7.27 (d, J = 2.0 Hz, 1H), 7.14 (dd, J = 8.1, 1.9 Hz, 1H), 6.78 (dd, J = 16.8, 10.6 Hz, 1H), 6.19 (dd, J = 16.8, 2.0 Hz, 1H), 5.74 (dd, J = 10.7, 2.0 Hz, 1H), 4.36 (d, J = 13.3 Hz, 1H), 4.05 (d, J = 13.4 Hz, 1H), 3.96 (s, 3H), 3.87 (s, 3H), 3.78 (ddt, J = 28.4, 19.1, 9.4 Hz, 4H), 3.24 (t, J = 12.3 Hz, 1H), 2.97 (t, J = 12.0 Hz, 1H), 2.61 (dt, J = 9.9, 5.7 Hz, 1H), 1.84 (d, J = 12.8 Hz, 2H), 1.26 (t, J = 11.0 Hz, 2H). | 523.20 |
| 1-[(1R,5S,6S)-6- [2-[4-amino-5-(4- chloro-3- methoxyphenyl)- 7-methyl-7H- pyrrolo[2,3- d]pyrimidin-6- yl]ethynyl]-3- azabicyclo[3.1.0] hexan-3-yl]prop-2- en-1-one | 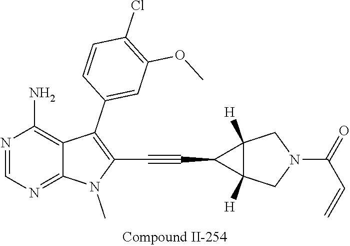 [SMILES: C=CC(=O)N1C[C@@H]2[C@@H](C#Cc3c(-c4ccc(Cl)c(OC)c4)c4c(N)ncnc4n3C)[C@@H]2C1] | ¹H NMR (400 MHz, DMSO-d₆) δ 8.19 (s, 1H), 7.53 (d, J = 8.1 Hz, 1H), 7.22 (d, J = 1.9 Hz, 1H), 7.06 (dd, J = 8.1, 1.9 Hz, 1H), 6.50 (dd, J = 16.7, 10.3 Hz, 1H), 6.28 (br, 1.3H), 6.11 (dd, J = 16.7, 2.4 Hz, 1H), 5.65 (dd, J = 10.3, 2.4 Hz, 1H), 3.91-3.87 (m, J = 15.1 Hz, 4H), 3.76 (d, J = 12.6 Hz, 1H), 3.70 (s, 3H), 3.64 (dd, J = 10.8, 4.2 Hz, 1H), 3.41 (dd, J = 12.6, 4.3 Hz, 1H), 2.15 − 2.09 (m, 1H), 2.05 (m, J = 7.5, 3.8 Hz, 1H), 1.52 (t, J = 3.4 Hz, 1H). | 448.15 |
| 1-((3R,4S)-4-(3- ((4-amino-5-(4- chloro-3- methoxyphenyl)- 7-methyl-7H- pyrrolo[2,3- d]pyrimidin-6- yl)ethynyl)azetidin- 1-yl)-3- hydroxypiperidin- 1-yl)prop-2-en-1- one | 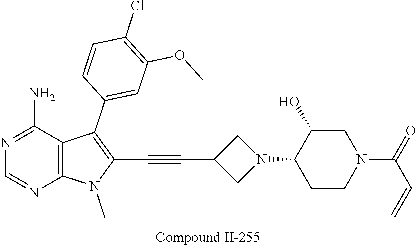 [SMILES: C=CC(=O)N1CC[C@H](N2CC(C#Cc3c(-c4ccc(Cl)c(OC)c4)c4c(N)ncnc4n3C)C2)[C@H](O)C1] | ¹H NMR (400 MHz, DMSO-d₆) δ 8.19 (s, 1H), 7.54 (d, J = 8.1 Hz, 1H), 7.23 (d, J = 1.9 Hz, 1H), 7.09 (dd, J = 8.1, 1.9 Hz, 1H), 6.84 − 6.73 (m, 1H), 6.25 (s, 2H), 6.05 (dd, J = 16.7, 2.5 Hz, 1H), 5.68 − 5.59 (m, 1H), 4.55 (d, J = 3.6 Hz, 1H), 4.02 (d, J = 13.2 Hz, 1H), 3.90 (s, 3H), 3.85 − 3.76 (m, 1H), 3.73 (s, 3H), 3.66 (d, J = 6.5 Hz, 1H), 3.59 (t, J = 6.5 Hz, 2H), 3.48 (q, J = 7.5 Hz, 1H), 3.20 (d, J = 13.3 Hz, 1H), 3.10 (dt, J = 14.0, 7.0 Hz, 2H), 2.89 (dt, J = 13.2, 6.6 Hz, 1H), 2.36 (s, 1H), 1.51 (s, 2H), | 521.3 |
| 1-((2S)-4-(3-((4- amino-5-(4- chloro-3- methoxyphenyl)- 7-methyl-7H- pyrrolo[2,3- d]pyrimidin-6- yl)ethynyl)azetidin- 1-yl)-2- (hydroxymethyl) piperidin-1-yl)prop- 2-en-1-one | 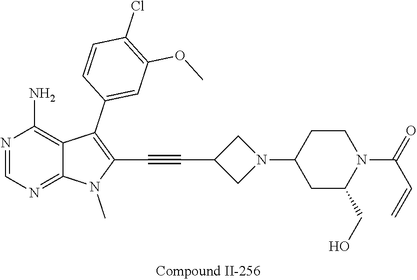 [SMILES: C=CC(=O)N1CCC(N2CC(C#Cc3c(-c4ccc(Cl)c(OC)c4)c4c(N)ncnc4n3C)C2)C[C@H]1CO] | ¹H NMR (400 MHz, DMSO-d₆) δ 8.20 (s, 1H), 7.54 (d, J = 8.1 Hz, 1H), 7.22 (d, J = 2.0 Hz, 1H), 7.10 (dd, J = 8.1, 1.9 Hz, 1H), 6.79 (dd, J = 16.8, 10.5 Hz, 1H), 6.25 (s, 2H),6.05 (dd, J = 16.8, 2.5 Hz, 1H), 5.61 (d, J = 10.0 Hz, 1H), 4.88 (s, 1H), 4.35 − 3.91 (m, 2H), 3.89 (s, 3H),3.74 (s, 3H), 3.56 − 3.45(m, 4H), 3.31 − 2.82 (m, 3H), 2.41 (s, 1H), 1.62 (m, 3H), 1.39 (s, 1H). | 535.3 |
| (2E)-1- [(1R,5S,6S)-6-[2- [4-amino-5-(4- chloro-3- methoxyphenyl)- 7-methyl-7H- pyrrolo[2,3- d]pyrimidin-6- ylethynyl]-3- azabicyclo[3.1.0] hexan-3-yl]-4- (dimethylamino) but-2-en-1-one | 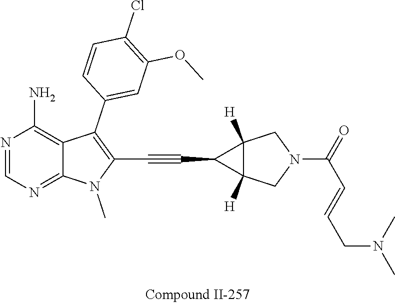 [SMILES: COc1cc(-c2c(C#C[C@@H]3[C@H]4CN(C(=O)/C=C/CN(C)C)C[C@@H]34)n(C)c3ncnc(N)c23)ccc1Cl] | ¹H NMR (400 MHz, DMSO-d₆) δ 8.19 (s, 1H), 7.54 (d, J = 8.1 Hz, 1H), 7.21 (d, J = 2.0 Hz, 1H), 7.06 (dd, J = 8.1, 1.9 Hz, 1H), 6.58 (dt, J = 15.2, 6.2 Hz, 1H), 6.28 (dt, J = 15.2, 1.6 Hz, 2.6H), 3.91 (s, 3H), 3.85 (d, J = 10.7 Hz, 1H), 3.75 (d, J = 12.5 Hz, 1H), 3.70 (s, 3H), 3.63 (dd, J = 10.7, 4.2 Hz, 1H), 3.39 (dd, J = 12.6, 4.3 Hz, 1H), 3.01 (dd, J = 6.3, 1.5 Hz, 2H), 2.14 (s, 6H), 2.12 − 2.09 (m, 1H), 2.06-2.03 (dt, J = 7.5, 3.8 Hz, 1H), 1.52 (t, J = 3.5 Hz, 1H). | 505.30 |
| 1-((3S,4S)-4-(3- ((4-amino-5-(4- chloro-3- methoxyphenyl)- 7-methyl-7H- pyrrolo[2,3- d]pyrimidin-6- yl)ethynyl)azetidin- 1-yl)-3- hydroxypiperidin- 1-yl)prop-2-en-1- one | 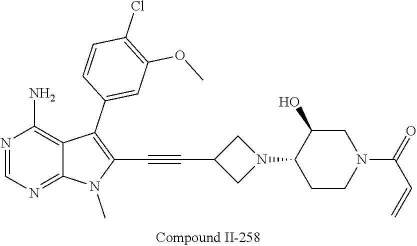 [SMILES: C=CC(=O)N1CC[C@H](N2CC(C#Cc3c(-c4ccc(Cl)c(OC)c4)c4c(N)ncnc4n3C)C2)[C@@H](O)C1] | ¹H NMR (400 MHz, DMSO-d₆) δ 8.20 (s, 1H), 7.53 (d, J = 8.1 Hz, 1H), 7.23 (d, J = 1.9 Hz, 1H), 7.09 (dd, J = 8.1, 1.9 Hz, 1H), 6.72 (dd, J = 17.0, 10.8 Hz, 1H), 6.25 (s, 2H), 6.06 (d, J = 16.7 Hz, 1H), 5.68 − 5.60 (d, 1H), 4.92 (s, 1H), 3.96 (m, 1H),3.91 (s, 3H), 3.73 (m, 4H), 3.67 (s, 1H), 3.58 (m, 3H), 3.46 − 2.85 (m, 4H), 2.20 (s, 1H), 1.70 (s, 1H), 1.09 (s, 1H). | 521.3 |
| 1-((3R,4S)-4-(3- ((4-amino-5-(4- chloro-3- methoxyphenyl)- 7-methyl-7H- pyrrolo[2,3- d]pyrimidin-6- yl)ethynyl)azetidin- 1-yl)-3- fluoropiperidin-1- yl)prop-2-en-1-one | 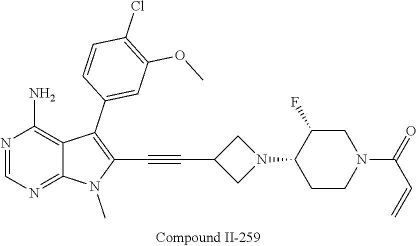 [SMILES: C=CC(=O)N1CC[C@H](N2CC(C#Cc3c(-c4ccc(Cl)c(OC)c4)c4c(N)ncnc4n3C)C2)[C@H](F)C1] | ¹H NMR (400 MHz, DMSO-d₆) δ 8.20 (s, 1H), 7.54 (d, J = 8.1 Hz, 1H), 7.23 (d, J = 1.9 Hz, 1H), 7.09 (dd, J = 8.1, 1.9 Hz, 1H), 6.79 (ddd, J = 24.1, 16.6, 10.5 Hz, 1H), 6.30, (br, 1 H), 6.09 (dd, J = 16.7, 2.4 Hz, 1H), 5.67 (d, J = 10.2 Hz, 1H), 4.73- 4.61 (m, 1H), 4.28-4.24 (d, J = 13.1 Hz, 1H), 3.90 (s, 3H), 3.74 (s, 3H), 3.64 − 3.56 (m, 2H), 3.51 (p, J = 7.4 Hz, 1H), 3.33 (m, 1H), 3.22 − 3.09 (m, 3H), 2.81 (t, J = 12.2 Hz, 1H), 2.43 (t, J = 11.7 Hz, 1H), 1.55 (d, J = 12.4 Hz, 1H), 1.45- 1.21 (m, 1H). | 523.25 |
| 1-((1R,5S,6s)-6- ((4-amino-5-(5- chloro-2- methoxyphenyl)- 7-methyl-7H- pyrrolo[2,3- d]pyrimidin-6- yl)ethynyl)-3- azabicyclo[3.1.0] hexan-3-yl)prop-2- en-1-one | 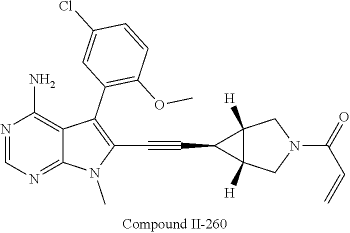 [SMILES: C=CC(=O)N1C[C@@H]2[C@@H](C#Cc3c(-c4cc(Cl)ccc4OC)c4c(N)ncnc4n3C)[C@@H]2C1] | ¹H NMR (400 MHz, DMSO-d₆) δ 8.15 (s, 1H), 7.46 (dd, J = 8.8, 2.7 Hz, 1H), 7.31 (d, J = 2.7 Hz, 1H), 7.18 (d, J = 8.9 Hz, 1H), 6.49 (dd, J = 16.9, 10.5 Hz, 1H), 6.10 (dd, J = 16.8, 2.4 Hz, 1H), 5.95 (s, 2H), 5.65 (dd, J = 10.3, 2.4 Hz, 1H), 3.87 (dd, J = 10.8, 4.2 Hz, 1H), 3.76 (s, 3H), 3.79 − 3.70 (m, 1H), 3.69 (s, 3H), 3.63 (dd, J = 11.5, 3.7 Hz, 1H), 3.39 (dt, J = 12.6, 3.5 Hz, 1H), 3.33 (s, 1H), 2.01 (tq, J = 24.1, 3.7 Hz, 3H), 1.46 (t, J = 3.4 Hz, 1H). | 448.1 |
| (E)-1-((1R,5S,6s)- 6-((4-amino-5-(5- chloro-2- methoxyphenyl)- 7-methyl-7H- pyrrolo[2,3- d]pyrimidin-6- yl)ethynyl)-3- azabicyclo[3.1.0] hexan-3-yl)-4- (dimethylamino) but-2-en-1-one | 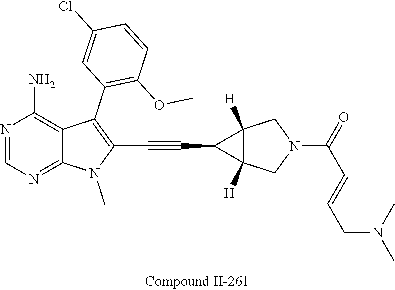 [SMILES: COc1ccc(Cl)cc1-c1c(C#C[C@@H]2[C@H]3CN(C(=O)/C=C/CN(C)C)C[C@@H]23)n(C)c2ncnc(N)c12] | ¹H NMR (400 MHz, DMSO-d₆) δ 8.15 (s, 1H), 7.46 (dd, J = 8.8, 2.7 Hz, 1H), 7.31 (d, J = 2.7 Hz, 1H), 7.18 (d, J = 8.9 Hz, 1H), 6.58 (dt, J = 15.1, 6.2 Hz, 1H), 6.27 (d, J = 15.2 Hz, 1H), 5.95 (s, , 2H), 3.84 (dd, J = 10.6, 4.2 Hz, 1H), 3.76 (s, 3H), 3.76 − 3.70 (m, 1H), 3.68 (s, 3H), 3.62 (d, J = 10.7 Hz, 1H), 3.37 (dd, J = 12.5, 4.2 Hz, 1H), 3.00 (d, J = 6.1 Hz, 2H), 2.14 (s, 6H), 2.08 − 1.90 (m, 2H), 1.45 (t, J = 3.4 Hz, 1H) | 505.2 |
| 1-(4-(3-((4-amino- 5-(2,2- difluorobenzo[d][1,3] dioxol-4-yl)-7- methyl-7H- pyrrolo[2,3- d]pyrimidin-6- yl)ethynyl)azetidin- 1-yl)piperidin-1- yl)prop-2-en-1-one | 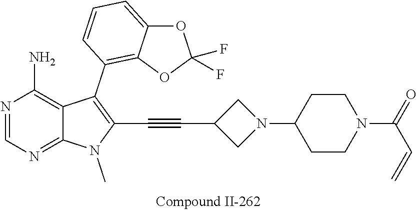 [SMILES: C=CC(=O)N1CCC(N2CC(C#Cc3c(-c4cccc5c4OC(F)(F)O5)c4c(N)ncnc4n3C)C2)CC1] | ¹H NMR (400 MHz, DMSO-d₆) δ 8.21 (s, 1H), 7.43 (dd, J = 8.1, 1.2 Hz, 1H), 7.31 (t, J = 8.0 Hz, 1H), 7.19 (dd, J = 8.0, 1.2 Hz, 1H), 6.79 (dd, J = 16.7, 10.5 Hz, 1H), 6.38 (s, 1H), 6.07 (dd, J = 16.7, 2.5 Hz, 1H), 5.65 (dd, J = 10.5, 2.5 Hz, 1H), 3.97 (d, J = 12.8 Hz, 1H), 3.81 (d, J = 13.6 Hz, 1H), 3.75 (s, 3H), 3.54 (t, J = 6.9 Hz, 2H), 3.43 (q, J = 7.5 Hz, 1H), 3.18 (t, J = 11.8 Hz, 1H), 2.97 (q, J = 7.8, 7.1 Hz, 3H), 2.31 − 2.17 (m, 1H), 1.59 (s, 2H), 1.06 (s, 2H). | 521.3 |
| 1-((3R,4S)-4-(3- ((4-amino-5-(2,2- difluorobenzo[d] [1,3]dioxol-4-yl)-7- methyl-7H- pyrrolo[2,3- d]pyrimidin-6- yl)ethynyl)azetidin- 1-yl)-3- hydroxypiperidin- 1-yl)prop-2-en-1- one | 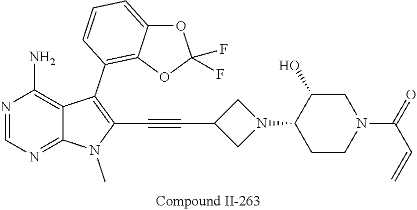 [SMILES: C=CC(=O)N1CC[C@H](N2CC(C#Cc3c(-c4cccc5c4OC(F)(F)O5)c4c(N)ncnc4n3C)C2)[C@H](O)C1] | ¹H NMR (400 MHz, DMSO-d₆) δ 8.21 (s, 1H), 7.42 (d, J = 8.0 Hz, 1H), 7.31 (t, J = 8.0 Hz, 1H), 7.19 (dd, J = 8.0, 1.2 Hz, 1H), 6.74 (ddd, J = 20.5, 16.8, 10.5 Hz, 1H), 6.36 (s, 2H), 6.04 (dd, J = 16.8, 2.5 Hz, 1H), 5.66 − 5.58 (m, 1H), 4.44 (dd, J = 50.1, 3.4 Hz, 1H), 4.00 (d, J = 12.5 Hz, 1H), 3.74 (s, 4H), 3.63 (s, 1H), 3.60 − 3.52 (m, 3H), 3.43 (q, J = 7.6 Hz, 1H), 3.19 (d, J = 13.4 Hz, 1H), 3.04 (dt, J = 14.2, 7.1 Hz, 2H), 2.89 (d, J = 13.1 Hz, 1H), 2.30 (d, J = 31.5 Hz, 1H), 1.36 (s, 2H). | 537.3 |
| 1-(4-(3-((4-amino- 5-(3-chloro-2- methoxyphenyl)- 7-methyl-7H- pyrrolo[2,3- d]pyrimidin-6- yl)ethynyl)azetidin- 1-yl)piperidin-1- yl)prop-2-en-1-one | 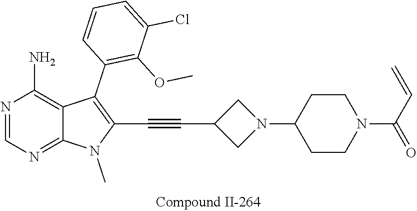 [SMILES: C=CC(=O)N1CCC(N2CC(C#Cc3c(-c4cccc(Cl)c4OC)c4c(N)ncnc4n3C)C2)CC1] | ¹H NMR (400 MHz, DMSO-d₆) δ 8.18 (s, 1H), 7.57 (d, J = 7.8 Hz, 1H), 7.32 (d, J = 7.8 Hz, 1H), 7.26 (t, J = 7.7 Hz, 1H), 6.79 (dd, J = 16.7, 10.5 Hz, 1H), 6.07 (dd, J = 16.8, 2.4 Hz, 1H), 5.95 (s, 2H), 5.65 (dd, J = 10.5, 2.5 Hz, 1H), 3.96 (d, J = 12.8 Hz, 1H), 3.81 (d, J = 13.9 Hz, 1H), 3.74 (s, 3H), 3.52 (t, J = 6.2 Hz, 2H), 3.42 (s, 3H), 3.36 (m, 1H), 3.21 (m, 1H), 3.01 (t, J = 11.2 Hz, 1H), 2.92 (q, J = 7.4 Hz, 2H), 2.23 (s, 1H), 1.59 (s, 2H), 1.07 (d, J = 13.2 Hz, 2H) | 505.2 |
| 1-((1R,5S,6s)-6- ((4-amino-5-(3- chloro-2- methoxyphenyl)- 7-methyl-7H- pyrrolo[2,3- d]pyrimidin-6- yl)ethynyl)-3- azabicyclo[3.1.0] hexan-3-yl)prop-2- en-1-one | 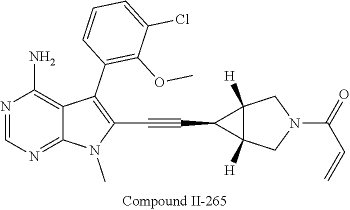 [SMILES: C=CC(=O)N1C[C@@H]2[C@@H](C#Cc3c(-c4cccc(Cl)c4OC)c4c(N)ncnc4n3C)[C@@H]2C1] | ¹H NMR (400 MHz, DMSO-d₆) δ 8.15 (s, 1H), 7.57 (dd, J = 7.6, 2.0 Hz, 1H), 7.33 − 7.21 (m, 2H), 6.48 (dd, J = 16.7, 10.3 Hz, 1H), 6.09 (dd, J = 16.8, 2.3 Hz, 1H), 5.95 (s, 2H), 5.64 (dd, J = 10.4, 2.4 Hz, 1H), 3.86 (d, J = 10.8 Hz, 1H), 3.78 − 3.69 (m, 1H), 3.71 (s, 3H), 3.62 (dd, J = 10.8, 4.3 Hz, 1H), 3.33 (s, 3H), 3.31 (s, 1H), 2.00 (ddt, J = 21.4, 7.4, 3.7 Hz, 2H), 1.43 (t, J = 3.4 Hz, 1H). | 448.1 |
| 1-[(3R,4S)-4-(3- [2-[4-amino-5-(3- chloro-2- methoxyphenyl)- 7-methyl-7H- pyrrolo[2,3- d]pyrimidin-6- yl]ethynyl]azetidin- 1-yl)-3- hydroxypiperidin- 1-yl]prop-2-en-1- one | 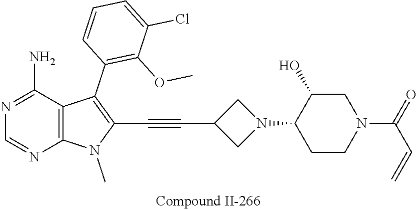 [SMILES: C=CC(=O)N1CC[C@H](N2CC(C#Cc3c(-c4cccc(Cl)c4OC)c4c(N)ncnc4n3C)C2)[C@H](O)C1] | ¹H NMR (400 MHz, DMSO-d₆) δ 8.18 (s, 1H), 7.57 (dd, J = 7.9, 1.8 Hz, 1H), 7.31 (dd, J = 7.6, 1.8 Hz, 1H), 7.25 (t, J = 7.7 Hz, 1H), 6.75 − 6.69 (m, 1H), 6.05 (dd, J = 16.7, 2.5 Hz, 2.3H), 5.62 (dt, J = 10.2, 3.5 Hz, 1H), 4.50-4.37 (dd, J = 49.5, 3.4 Hz, 1H), 3.99 − 3.76 (m, 1H), 3.74 (s, 3H), 3.62 − 3.55 (m, 4H), 3.47 (s, 3H), 3.37 (t, J = 7.6 Hz, 1H), 3.30 − 3.18 (m, 1H), 3.00 (p, J = 7.2, 6.5 Hz, 2H), 2.94 − 2.83 (m, 1H), 2.33-2.25 (m, 1H), 1.49-1.36 (d, J = 50.9 Hz, 2H). | 521.25 |
| (E)-1-((1R,5S,6s)- 6-((4-amino-5-(3- chloro-2- methoxyphenyl)- 7-methyl-7H- pyrrolo[2,3- d]pyrimidin-6- yl)ethynyl)-3- azabicyclo[3.1.0] hexan-3-yl)-4- (dimethylamino) but-2-en-1-one | 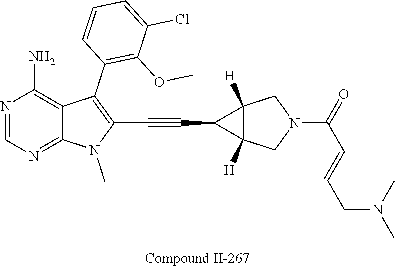 [SMILES: COc1c(Cl)cccc1-c1c(C#C[C@@H]2[C@H]3CN(C(=O)/C=C/CN(C)C)C[C@@H]23)n(C)c2ncnc(N)c12] | ¹H NMR (400 MHz, DMSO-d₆) δ 8.17 (s, 1H), 7.57 (dd, J = 7.7, 2.0 Hz, 1H), 7.33 − 7.21 (m, 2H), 6.57 (dt, J = 15.0, 6.1 Hz, 1H), 6.26 (d, J = 15.2 Hz, 1H), 5.95(s, 2H), 3.82 (d, J = 10.7 Hz, 1H), 3.71 (m, J = 12.5 Hz, 4H), 3.61 (dd, J = 10.8, 4.3 Hz, 1H), 3.45 (s, 3H), 3.36 (dd, J = 12.6, 4.3 Hz, 1H), 3.00 (d, J = 6.2 Hz, 2H), 2.13 (s, 6H), 2.02 (s, 1H), 1.92 (m, 1H), 1.43 (t, J = 3.4 Hz, 1H). | |
| 1-(4-(3-((4-amino- 5-(2- methoxyphenyl)- 7-methyl-7H- pyrrolo[2,3- d]pyrimidin-6- yl)ethynyl)azetidin- 1-yl)piperidin-1- yl)prop-2-en-1-one | 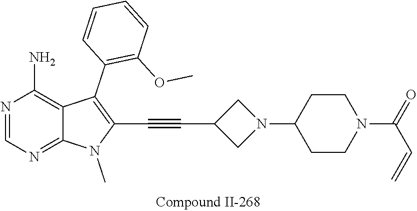 [SMILES: C=CC(=O)N1CCC(N2CC(C#Cc3c(-c4ccccc4OC)c4c(N)ncnc4n3C)C2)CC1] | ¹H NMR (400 MHz, DMSO-d₆) δ 8.15 (s, 1H), 7.43 (t, J = 7.9 Hz, 1H), 7.31 (d, J = 7.4 Hz, 1H), 7.17 (d, J = 8.3 Hz, 1H), 7.07 (t, J = 7.4 Hz, 1H), 6.78 (dd, J = 16.8, 10.5 Hz, 1H), 6.06 (dd, J = 16.3, 2.4 Hz, 1H), 5.68 − 5.60 (m, 1H), 3.96 (d, J = 12.8 Hz, 1H), 3.76 (s, 4H), 3.71 (s, 3H), 3.53 (s, 2H), 3.36 (t, J = 7.6 Hz, 1H), 3.18 (s, 1H), 2.94 (q, J = 6.9 Hz, 3H), 2.24 (s, 1H), 1.59 (s, 2H), 1.06 (s, 2H). | 471.3 |
| 1-((1R,5S,6s)-6- ((4-amino-5-(2- methoxyphenyl)- 7-methyl-7H- pyrrolo[2,3- d]pyrimidin-6- yl)ethynyl)-3- azabicyclo[3.1.0] hexan-3-yl)prop-2- en-1-one | 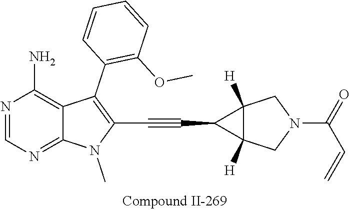 [SMILES: C=CC(=O)N1C[C@@H]2[C@@H](C#Cc3c(-c4ccccc4OC)c4c(N)ncnc4n3C)[C@@H]2C1] | ¹H NMR (400 MHz, DMSO-d₆) δ 8.15 (s, 1H), 7.44 (t, J = 7.9 Hz, 1H), 7.30 (d, J = 7.4 Hz, 1H), 7.18 (d, J = 8.3 Hz, 1H), 7.09 (t, J = 7.4 Hz, 1H), 6.49(m, J = 16.8, 10.4 Hz, 1H), 6.10 (dd, J = 16.7, 2.3 Hz, 1H), 5.85 (s, 2H), 5.68 − 5.61 (m, 1H), 3.86 (d, J = 10.8 Hz, 1H), 3.73 (d, J = 27.3 Hz, 7H), 3.62 (dd, J = 10.8, 4.2 Hz, 1H), 3.38 (dd, J = 12.5, 4.4 Hz, 1H), 2.05 − 1.94 (m, 2H), 1.42 (s, 1H). | 414.2 |
| 1-((1R,5S,6s)-6- ((4-amino-5-(4- chloro-3- methoxyphenyl)- 7-methyl-7H- pyrrolo[2,3- d]pyrimidin-6- yl)ethynyl)-3- azabicyclo[3.1.0] hexan-3-yl)prop-2- en-1-one | 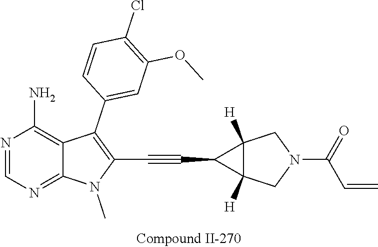 [SMILES: C=CC(=O)N1C[C@@H]2[C@@H](C#Cc3c(-c4ccc(Cl)c(OC)c4)c4c(N)ncnc4n3C)[C@@H]2C1] | ¹H NMR (400 MHz, DMSO-d₆) δ 8.02-8.29 (m, 2H), 7.52 (d, J =8.30 Hz, 1H), 7.20 (d, J =1.71 Hz, 1H), 6.97-7.10 (m, 2H), 6.39-6.59 (m, 2H), 5.99-6.16 (m, 2H), 5.53-5.69 (m, 1H), 3.87-3.92 (m, 3H), 3.71- 3.80 (m, 1H), 3.67-3.71 (m, 3H), 3.59-3.66 (m, 1H), 3.36-3.43 (m, 1H), 2.07-2.14 (m, 1H), 2.00-2.07 (m, 1H), 1.44-1.55 (m, 1H). | 448 |
| 1-((1R,5S,6s)-6- ((4-amino-5-(4- chloro-3- methoxyphenyl)- 7-isopropyl-7H- pyrrolo[2,3- d]pyrimidin-6- yl)ethynyl)-3- azabicyclo[3.1.0] hexan-3-yl)prop-2- en-1-one | 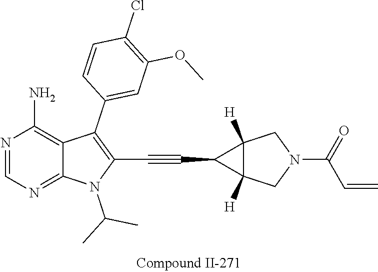 [SMILES: C=CC(=O)N1C[C@@H]2[C@@H](C#Cc3c(-c4ccc(Cl)c(OC)c4)c4c(N)ncnc4n3C(C)C)[C@@H]2C1] | ¹H NMR (400 MHz, DMSO-d₆) δ 8.14 (s, 1H), 7.51 (d, J =8.06 Hz, 2H), 7.20 (d, J =1.95 Hz, 1H), 7.04 (dd, J =1.95, 8.06 Hz, 2H), 6.48 (dd, J =10.25, 16.85 Hz, 2H), 5.99-6.18 (m, 2H), 5.75 (s, 2H), 5.57-5.70 (m, 1H), 4.93-5.16 (m, 2H), 3.83-3.97 (m, 4H), 3.68-3.79 (m, 2H), 3.56- 3.67 (m, 2H), 3.39 (br dd, J-4.27, 12.57 Hz, 2H), 1.92-2.15 (m, 3H), 1.59 (d, J =6.84 Hz, 6H), 1.48-1.53 (m, 1H). | 476 |
| 1-((1R,5S,6s)-6- ((4-amino-5-(4- bromo-3- methoxyphenyl)- 7-isopropyl-7H- pyrrolo[2,3- d]pyrimidin-6- yl)ethynyl)-3- azabicyclo[3.1.0] hexan-3-yl)prop-2- en-1-one | 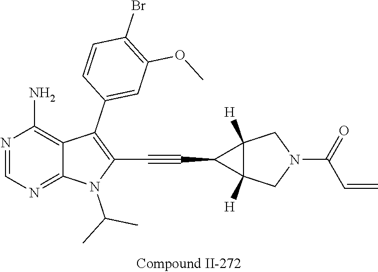 [SMILES: C=CC(=O)N1C[C@@H]2[C@@H](C#Cc3c(-c4ccc(Br)c(OC)c4)c4c(N)ncnc4n3C(C)C)[C@@H]2C1] | ¹H NMR (400 MHz, DMSO-d₆) δ 8.02-8.29 (m, 2H), 7.66 (d, J =8.06 Hz, 1H), 7.17 (d, J =1.95 Hz, 1H), 6.98 (dd, J =1.83, 8.18 Hz, 1H), 6.39-6.60 (m, 2H), 6.03-6.19 (m, 2H), 5.70-5.81 (m, 2H), 5.58-5.70 (m, 1H), 4.96-5.17 (m, 2H), 3.89 (s, 4H), 3.70-3.80 (m, 2H), 3.55-3.67 (m, 1H), 3.35-3.46 (m, 1H), 1.92- 2.12 (m, 3H), 1.55-1.64 (m, 6H), 1.48-1.54 (m, 1H). | 520 |
| 1-(6-((4-amino-5- (4-bromo-2- fluoro-3- methoxyphenyl)- 7-isopropyl-7H- pyrrolo[2,3- d]pyrimidin-6- yl)ethynyl)-3- azabicyclo[3.1.0] hexan-3-yl)prop-2- en-1-one | 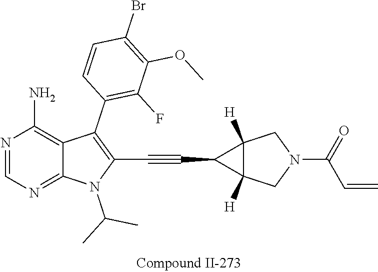 [SMILES: C=CC(=O)N1C[C@@H]2[C@@H](C#Cc3c(-c4ccc(Br)c(OC)c4F)c4c(N)ncnc4n3C(C)C)[C@@H]2C1] | ¹H NMR (500 MHz, DMSO-d6): δ 8.03-8.19 (m, 1H), 7.44-7.58 (m, 1H), 7.02-7.13 (m, 1H), 6.36-6.56 (m, 1H), 6.14-6.33 (m, 1H), 5.99- 6.14 (m, 1H), 5.53-5.69 (m, 1H), 4.97-5.18 (m, 1H), 3.98-4.07 (m, 1H), 3.90-3.95 (m, 3H), 3.82-3.90 (m, 1H), 3.69-3.75 (m, 1H), 3.57- 3.65 (m, 1H), 3.34-3.41 (m, 1H), 1.99-2.06 (m, 1H), 1.91-1.96 (m, 1H), 1.53-1.65 (m, 6H), 1.19-1.31 (m, 1H). | 538 |
| 1-((3R,4S)-4-(3- ((4-amino-5-(2- methoxyphenyl)- 7-methyl-7H- pyrrolo[2,3- d]pyrimidin-6- yl)ethynyl)azetidin- 1-yl)-3- hydroxypiperidin- 1-yl)prop-2-en-1- one | 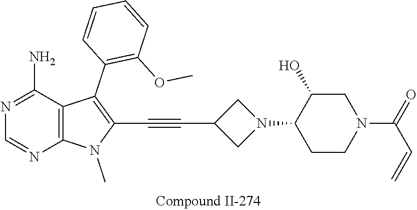 [SMILES: C=CC(=O)N1CC[C@H](N2CC(C#Cc3c(-c4ccccc4OC)c4c(N)ncnc4n3C)C2)[C@H](O)C1] | ¹H NMR (400 MHz, DMSO-d₆) δ 8.15 (s, 1H), 7.43 (t, J = 7.9 Hz, 1H), 7.32 (d, J = 7.4 Hz, 1H), 7.17 (d, J = 8.4 Hz, 1H), 7.07 (t, J = 7.4 Hz, 1H), 6.74 (dd, J = 17.2, 10.8 Hz, 1H), 6.05 (d, J = 16.8 Hz, 1H), 5.88 (s, 1H), 5.62 (d, J = 10.5 Hz, 1H), 4.42 (d, 1H), 4.02 (m, 1H), 3.78 (s, , 3H), 3.72 (s, 3H), 3.61 (m, 3H), 3.39 (m, 1H), 3.18 (d, 2H), 2.89 (s, 1H), 2.29 (s, 1H), 1.39 (s, 2H). | 487.2 |
| (E)-1-((1R,5S,6s)- 6-((4-amino-5-(2- methoxyphenyl)- 7-methyl-7H- pyrrolo[2,3- d]pyrimidin-6- yl)ethynyl)-3- azabicyclo[3.1.0] hexan-3-yl)-4- (dimethylamino) but-2-en-1-one | 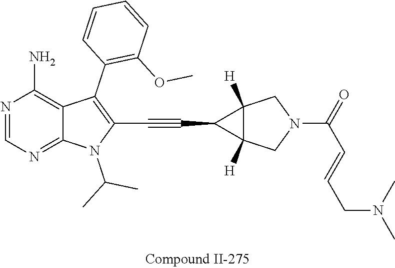 [SMILES: COc1ccccc1-c1c(C#C[C@@H]2[C@H]3CN(C(=O)/C=C/CN(C)C)C[C@@H]23)n(C(C)C)c2ncnc(N)c12] | ¹H NMR (400 MHz, DMSO-d₆) δ 8.16 (s, 1H), 7.45 (t, J = 7.8 Hz, 1H), 7.30 (d, J = 7.3 Hz, 1H), 7.18 (d, J = 8.6 Hz, 1H), 7.08 (t, J = 7.4 Hz, 1H), 6.64 − 6.52 (m, 1H), 6.26 (d, J = 15.2 Hz, 1H), 5.85 (s, 2H),3.84 (d, J = 10.8 Hz, 1H), 3.79(s, 3H), 3.76 (s, 4H), 3.61 (dd, J = 11.0, 4.2 Hz, 1H), 3.41 − 3.33 (m, 1H), 3.00 (d, J = 6.2 Hz, 2H), 2.14 (s, 6H), 1.96 (d, J = 9.2 Hz, 2H), 1.42 (d, J = 3.5 Hz, 1H). | 471.3 |
| 1-((1R,5S,6s)-6- ((4-amino-5-(2- methoxy-4-((6- methylpyridin-2- yl)oxy)phenyl)-7- (2-methoxyethyl)- 7H-pyrrolo[2,3- d]pyrimidin-6- yl)ethynyl)-3- azabicyclo[3.1.0] hexan-3-yl)prop-2- en-1-one | 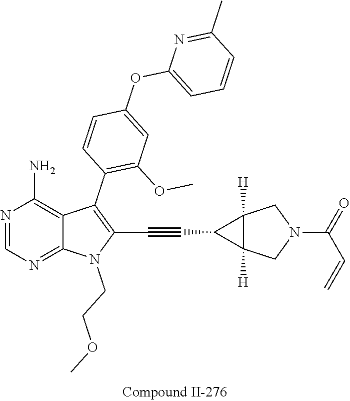 [SMILES: C=CC(=O)N1C[C@@H]2[C@@H](C#Cc3c(-c4ccc(Oc5cccc(C)n5)cc4OC)c4c(N)ncnc4n3CCOC)[C@@H]2C1] | ¹H NMR (400 MHz, DMSO-d₆) δ 8.19 (s, 1H), 7.78 (t, J = 7.8 Hz, 1H), 7.31 (d, J = 8.2 Hz, 1H), 7.06 (d, J = 7.4 Hz, 1H), 6.97 (s, 1H), 6.86 (d, J = 8.2 Hz, 1H), 6.78 (d, J = 8.3 Hz, 1H), 6.49 (dd, J = 16.7, 10.3 Hz, 1H), 6.25(s, 2H), 6.10 (d, J = 16.8 Hz, 1H), 5.65 (d, J = 10.4 Hz, 1H), 4.36 (t, J = 5.4 Hz, 2H), 3.88 (d, J = 10.8 Hz, 1H), 3.73 (d, J = 6.7 Hz, 6H), 3.62 (dd, J = 10.9, 4.2 Hz, 1H), 3.39 (d, J = 4.2 Hz, 1H), 3.25 (s, 3H), 2.39 (s, 3H), 2.05 (s, 2H), 1.46 (d, J = 3.8 Hz, 1H). | 565.3 |
| 1-((1R,5S,6s)-6- ((4-amino-5-(3- methoxy-4-((6- methylpyridin-2- yl)oxy)phenyl)-7- (2-methoxyethyl)- 7H-pyrrolo[2,3- d]pyrimidin-6- yl)ethynyl)-3- azabicyclo[3.1.0] hexan-3-yl)prop-2- en-1-one | 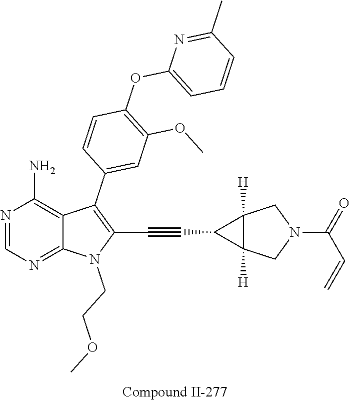 [SMILES: C=CC(=O)N1C[C@@H]2[C@@H](C#Cc3c(-c4ccc(Oc5cccc(C)n5)c(OC)c4)c4c(N)ncnc4n3CCOC)[C@@H]2C1] | ¹H NMR (400 MHz, DMSO-d₆) δ 8.19 (d, J = 1.4 Hz, 1H), 7.69 (t, J = 7.9 Hz, 1H), 7.29 − 7.20 (m, 2H), 7.09 (d, J = 8.1 Hz, 1H), 6.96 (d, J = 7.3 Hz, 1H), 6.74 (d, J = 8.2 Hz, 1H), 6.50 (dd, J = 16.8, 10.3 Hz, 1H), 6.28 (s, 2H), 6.15 − 6.06 (m, 1H), 5.69 − 5.62 (m, 1H), 4.37 (t, 2H), 3.89 (d, J = 10.8 Hz, 1H), 3.76 (d, J = 4.5 Hz, 6H), 3.64 (dd, J = 10.8, 4.2 Hz, 1H), 3.38 (m, 1H), 3.25 (S, 3H), 2.33 (s, 3H), 2.15 − 2.03 (m, 2H), 1.53 (t, J = 3.3 Hz, 1H) | 565.4 |
| 1-((1R,5S,6s)-6- ((4-amino-7-(3- hydroxypropyl)-5- (3-methoxy-4-((6- methylpyridin-2- yl)oxy)phenyl)- 7H-pyrrolo[2,3- d]pyrimidin-6- yl)ethynyl)-3- azabicyclo[3.1.0] hexan-3-yl)prop-2- en-1-one | 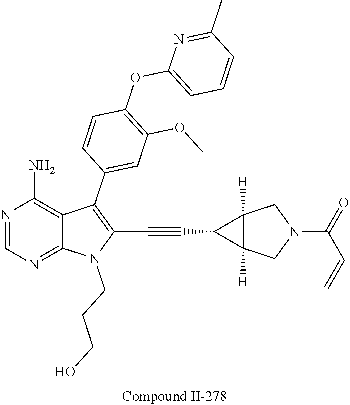 [SMILES: C=CC(=O)N1C[C@@H]2[C@@H](C#Cc3c(-c4ccc(Oc5cccc(C)n5)c(OC)c4)c4c(N)ncnc4n3CCCO)[C@@H]2C1] | ¹H NMR (400 MHz, DMSO-d₆) δ 8.19 (d, J = 1.4 Hz, 1H), 7.69 (t, J = 7.9 Hz, 1H), 7.29 − 7.20 (m, 2H), 7.09 (d, J = 8.1 Hz, 1H), 6.96 (d, J = 7.3 Hz, 1H), 6.74 (d, J = 8.2 Hz, 1H), 6.50 (dd, J = 16.8, 10.3 Hz, 1H), 6.28 (s, 2H), 6.15 − 6.06 (m, 1H), 5.69 − 5.62 (m, 1H), 4.63 (t, J = 5.2 Hz, 1H), 4.27 (t, J = 7.3 Hz, 2H), 3.89 (d, J = 10.8 Hz, 1H), 3.76 (d, J = 4.5 Hz, 4H), 3.64 (dd, J = 10.8, 4.2 Hz, 1H), 3.51 − 3.35 (m, 3H), 2.33 (s, 3H), 2.15 − 2.03 (m, 2H), 1.90 (q, J = 6.9 Hz, 2H), 1.52 (t, J = 3.3 Hz, 1H). | 565.4 |
| 1-((1R,5S,6s)-6- ((4-amino-5-(3- methoxy-4-((6- methylpyridin-2- yl)oxy)phenyl)-7- (3- (methylsulfonyl) propyl)-7H- pyrrolo[2,3- d]pyrimidin-6- yl)ethynyl)-3- azabicyclo[3.1.0] hexan-3-yl)prop-2- en-1-one | 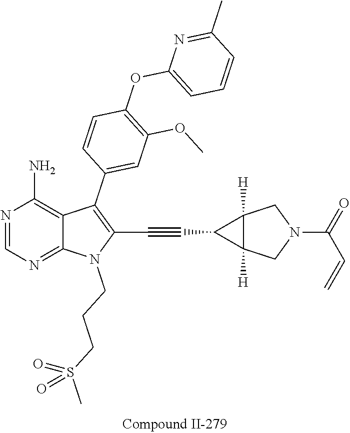 [SMILES: C=CC(=O)N1C[C@@H]2[C@@H](C#Cc3c(-c4ccc(Oc5cccc(C)n5)c(OC)c4)c4c(N)ncnc4n3CCCS(C)(=O)=O)[C@@H]2C1] | ¹H NMR (400 MHz, DMSO-d₆) δ 8.21 (s, 1H), 7.69 (t, J = 7.7 Hz, 1H), 7.29 − 7.21 (m, 2H), 7.10 (d, J = 8.2 Hz, 1H), 6.96 (d, J = 7.4 Hz, 1H), 6.74 (d, J = 8.2 Hz, 1H), 6.50 (dd, J = 16.8, 10.3 Hz, 1H), 6.25 (s, 2H), 6.10 (d, J = 16.8 Hz, 1H), 5.66 (d, J = 10.4 Hz, 1H), 4.35 (t, J = 7.0 Hz, 2H), 3.89 (d, J = 10.7 Hz, 1H), 3.76 (d, J = 12.4 Hz, 1H), 3.76 (s, 3H), 3.64 (dd, J = 10.8, 4.2 Hz, 1H), 3.41 (d, J = 4.4 Hz, 1H), 3.23 − 3.15 (m, 2H), 3.01 (s, 3H), 2.33 (s, 3H), 2.20 (t, J = 7.4 Hz, 1H), 2.15 (s, 2H), 2.09 (d, J = 5.3 Hz, 1H), 1.54 (d, J = 3.9 Hz, 1H). | 627.3 |
| (3S,4R)-4-(3-((4- amino-7-methyl-5- (4- phenoxyphenyl)- 7H-pyrrolo[2,3- d]pyrimidin-6- yl)ethynyl)azetidin- 1-yl)-1- (vinylsulfonyl) piperidin-3- ol formate | 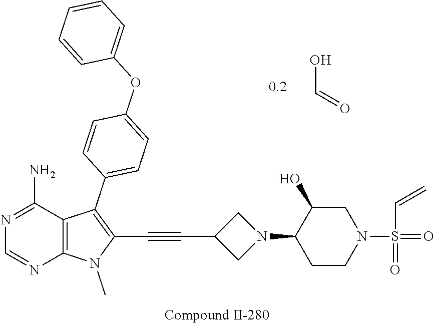 [SMILES: C=CS(=O)(=O)N1CC[C@@H](N2CC(C#Cc3c(-c4ccc(Oc5ccccc5)cc4)c4c(N)ncnc4n3C)C2)[C@@H](O)C1 ; O=CO] | ¹H NMR (400 MHz, DMSO-d₆) δ 8.18 (d, J = 5.1 Hz, 1.2H), 7.52 (d, J = 8.3 Hz, 2H), 7.43 (t, J = 7.8 Hz, 2H), 7.23 − 7.10 (m, 5H), 6.80 (dd, J = 16.5, 10.0 Hz, 1H), 6.11 (d, J = 10.0 Hz, 1H), 6.07 (d, J = 16.5 Hz, 2H), 4.78 (s, 1H), 3.72 (s, 3H), 3.64 (s, 3H), 3.56 (t, J = 6.8 Hz, 1H), 3.42 (t, J = 7.6 Hz, 1H), 3.20 − 2.95 (m, 3H), 2.90 (s, 1H), 2.51 (s, 2H), 2.30 (s, 1H), 1.56 (s, 1H), 1.44 (s, 1H). | 585.35 |
| 7-methyl-5-(4- phenoxyphenyl)-6- ((1-(1- (vinylsulfonyl) piperidin-4- yl)azetidin-3- yl)ethynyl)-7H- pyrrolo[2,3- d]pyrimidin-4- amine | 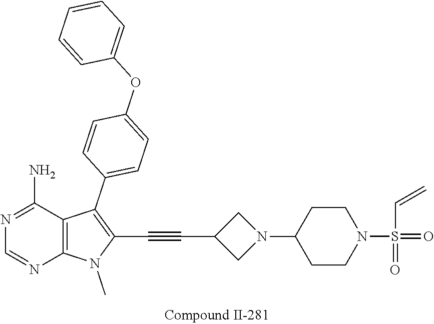 [SMILES: C=CS(=O)(=O)N1CCC(N2CC(C#Cc3c(-c4ccc(Oc5ccccc5)cc4)c4c(N)ncnc4n3C)C2)CC1] | ¹H NMR (400 MHz, DMSO-d₆) δ 8.19 (s, 1H), 7.52 (d, J = 8.2 Hz, 2H), 7.43 (t, J = 7.7 Hz, 2H), 7.18 (t, J = 7.3 Hz, 1H), 7.15 − 7.09 (m, 4H), 6.78 (dd, J = 16.5, 10.1 Hz, 1H), 6.27 (br, 1H), 6.15 (d, J = 10.1 Hz, 1H), 6.09 (d, J = 16.5 Hz, 1H), 3.72 (s, 3H), 3.54 (t, J = 7.1 Hz, 2H), 3.46 − 3.37 (m, 1H), 3.26 (d, J = 12.7 Hz, 3H), 2.98 (t, J = 6.9 Hz, 2H), 2.78 (t, J = 10.0 Hz, 2H), 2.14 (s, 1H), 1.63 (s, 2H), 1.23 (d, J = 10.3 Hz, 2H). | 569.35 |
| 1-((3R,4R)-3-(3- ((4-amino-5-(4- chloro-3- methoxyphenyl)- 7-methyl-7H- pyrrolo[2,3- d]pyrimidin-6- yl)ethynyl)azetidin- 1-yl)-4- hydroxypiperidin- 1-yl)prop-2-en-1- one | 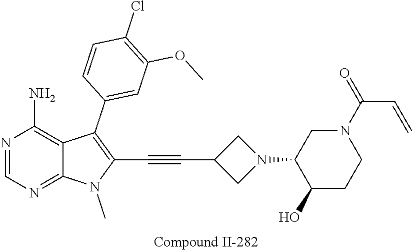 [SMILES: C=CC(=O)N1CC[C@@H](O)[C@H](N2CC(C#Cc3c(-c4ccc(Cl)c(OC)c4)c4c(N)ncnc4n3C)C2)C1] | ¹H NMR (400 MHz, DMSO-d₆) δ 8.20 (s, 1H), 7.53 (d, J = 8.1 Hz, 1H), 7.23 (d, J = 2.0 Hz, 1H), 7.09 (dd, J = 8.1, 1.9 Hz, 1H), 6.78 (dd, J = 16.8, 9.8 Hz, 1H), 6.30 (s, 2H), 6.13 − 6.03 (m, 1H), 5.66 (d, J = 10.4 Hz, 1H), 4.80 (s, 1H), 3.91 (s, 3H), 3.73 (s, 3H), 3.55 (m, 6H), 3.18 (m, 3H), 2.06 (s, 1H), 1.71 (s, 1H), 1.33 (s, 1H). | 521.2 |
| 1-((3R,4S)-4-(3- ((4-amino-5-(4- chloro-3- methylphenyl)-7- ethyl-7H- pyrrolo[2,3- d]pyrimidin-6- yl)ethynyl)azetidin- 1-yl)-3- fluoropiperidin-1- yl)prop-2-en-1-one | 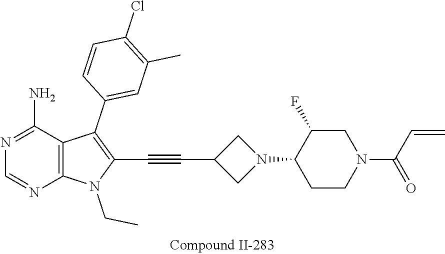 [SMILES: C=CC(=O)N1CC[C@H](N2CC(C#Cc3c(-c4ccc(Cl)c(C)c4)c4c(N)ncnc4n3CC)C2)[C@H](F)C1] | ¹H NMR (400 MHz, DMSO-d₆) δ 8.19 (s, 1H), 7.56 − 7.48 (m, 2H), 7.34 (dd, J = 8.2, 2.2 Hz, 1H), 6.79 (ddd, J = 23.3, 16.5, 10.3 Hz, 2H), 6.09 (br, 2.4 Hz, 1H), 5.68 (d, J = 10.4 Hz, 1H), 4.68 (d, J = 48.8 Hz, 2H), 4.26 (q, J = 7.0 Hz, 4H), 3.66 − 3.57 (m, 2H), 3.49 (q, J = 7.4 Hz, 1H), 3.20 − 3.06 (m, 3H), 2.82 (t, J = 11.7 Hz, 1H), 2.40 (s, 4H), 1.54 (s, 1H), 1.33 (t, J = 7.1 Hz, 4H). | 521.3 |
| 1-((3R,4S)-4-(3- ((4-amino-5-(4- chloro-3- methoxyphenyl)- 7-ethyl-7H- pyrrolo[2,3- d]pyrimidin-6- yl)ethynyl)azetidin- 1-yl)-3- hydroxypiperidin- 1-yl)prop-2-en-1- one | 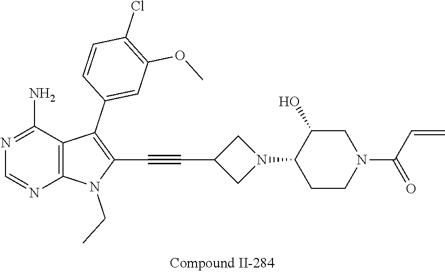 [SMILES: C=CC(=O)N1CC[C@H](N2CC(C#Cc3c(-c4ccc(Cl)c(OC)c4)c4c(N)ncnc4n3CC)C2)[C@H](O)C1] | ¹H NMR (400 MHz, DMSO-d₆) δ 8.19 (s, 1H), 7.53 (d, J = 8.1 Hz, 1H), 7.23 (d, J = 1.9 Hz, 1H), 7.09 (dd, J = 8.1, 1.9 Hz, 1H), 6.73 (ddd, J = 21.1, 16.7, 10.5 Hz, 1H), 6.30 (s, 2H), 6.05 (dt, J = 16.7, 2.4 Hz, 1H), 5.63 (ddd, J = 10.5, 5.3, 2.5 Hz, 1H), 4.50 (dd, J = 53.7, 3.6 Hz, 1H), 4.27 (q, J = 7.1 Hz, 2H), 4.00 (d, J = 12.8 Hz, 1H), 3.91 (s, 3H), 3.80 (dd, J = 13.6, 5.1 Hz, 1H), 3.67 (d, J = 7.4 Hz, 1H), 3.59 (d, J = 7.1 Hz, 2H), 3.51 − 3.35 (m, 1H), 3.32 − 3.17 (m, 1H), 3.09 (dt, J = 13.9, 7.0 Hz, 2H), 2.91 (dt, J = 13.3, 6.7 Hz, 1H), 2.32 (d, J = 32.4 Hz, 1H), 1.36 (dt, J = 14.3, 6.2 Hz, 5H). | 535.2 |
| 1-((3R,4S)-4-(3- ((4-amino-5-(7- fluorobenzo[d][1,3] dioxol-4-yl)-7- isopropyl-7H- pyrrolo[2,3- d]pyrimidin-6- yl)ethynyl)azetidin- 1-yl)-3- hydroxypiperidin- 1-yl)prop-2-en-1- one | 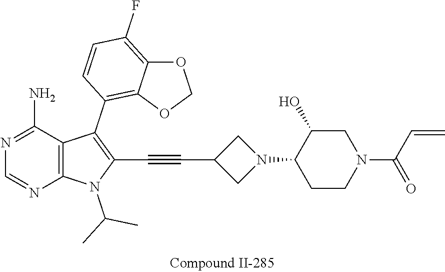 [SMILES: C=CC(=O)N1CC[C@H](N2CC(C#Cc3c(-c4ccc(F)c5c4OCO5)c4c(N)ncnc4n3C(C)C)C2)[C@H](O)C1] | ¹H NMR (400 MHz, DMSO-d₆) δ 8.14 (s, 1H), 7.00 − 6.85 (m, 2H), 6.76 (ddd, J = 20.4, 16.8, 10.5 Hz, 1H), 6.38 − 5.94 (m, 5H), 5.68 − 5.59 (m, 1H), 5.09 (t, J = 6.9 Hz, 1H), 4.50 (d, J = 54.6 Hz, 1H), 4.07 − 3.74 (m, 1H), 3.70 − 3.56 (m, 4H), 3.41 (s, 1H), 3.24 − 2.85 (m, 4H), 2.30 (d, J = 33.2 Hz, 1H), 2.08 (s, 1H), 1.61 (d, J = 6.9 Hz, 6H), 1.37 (d, J = 6.2 Hz, 2H). | 547.3 |
| 1-[(3R,4S)-4-(3- [2-[4-amino-5-(7- fluoro-2H-1,3- benzodioxol-4-yl)- 7-(propan-2-yl)- 7H-pyrrolo [2,3- d]pyrimidin-6- yl]ethynyl)azetidin- 1-yl)-3- fluoropiperidin-1- yl]prop-2-en-1-one | 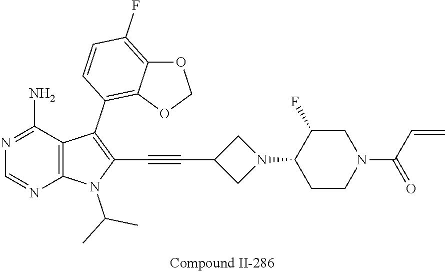 [SMILES: C=CC(=O)N1CC[C@H](N2CC(C#Cc3c(-c4ccc(F)c5c4OCO5)c4c(N)ncnc4n3C(C)C)C2)[C@H](F)C1] | ¹H NMR (400 MHz, DMSO-d₆) δ 8.14 (s, 1H), 6.96 (dd, J = 9.9, 8.9 Hz, 1H), 6.88 (dd, J = 8.9, 5.1 Hz, 1H), 6.82 − 6.73 (m, 1H), 6.17 − 6.07 (m, 4.9H), 5.69 − 5.66 (m, 1H), 5.08 (p, J = 6.9 Hz, 1H), 4.67 (d, J = 48.8 Hz, 1H), 4.47 − 4.23 (m, 1H), 4.20 − 3.86 (m, 1H), 3.58 (d, J = 7.9 Hz, 2H), 3.46 (m, J = 7.4 Hz, 1H), 3.15 (t, J = 13.4 Hz, 1H), 3.06 (d, J = 12.9 Hz, 2H), 2.80 (t, J = 11.8 Hz, 1H), 2.41 (m, 1H), 1.61 (d, J = 6.9 Hz, 6H), 1.55 (d, J = 12.6 Hz, 1H), 1.48 − 1.23 (m, 1H). | 549.35 |
| 1-((3R,4S)-4-(3- ((4-amino-5-(7- fluorobenzo[d][1,3] dioxol-4-yl)-7- isopropyl-7H- pyrrolo[2,3- d]pyrimidin-6- yl)ethynyl)azetidin- 1-yl)-3- fluoropiperidin-1- yl)prop-2-en-1-one | 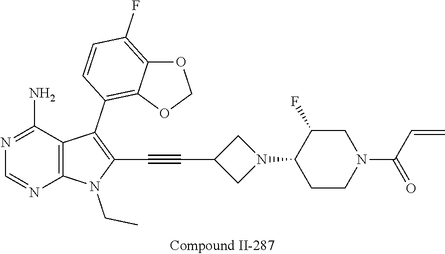 [SMILES: C=CC(=O)N1CC[C@H](N2CC(C#Cc3c(-c4ccc(F)c5c4OCO5)c4c(N)ncnc4n3CC)C2)[C@H](F)C1] | ¹H NMR (400 MHz, DMSO-d₆) δ 8.16 (s, 1H), 6.96 (dd, J = 10.0, 8.8 Hz, 1H), 6.89 (dd, J = 8.9, 5.0 Hz, 1H), 6.85 − 6.72 (m, 1H), 6.27 (s, 1H), 6.18 − 6.05 (m, 3H), 5.68 (ddd, J = 10.5, 4.6, 2.4 Hz, 1H), 4.67 (d, J = 48.7 Hz, 1H), 4.48 − 4.21 (m, 3H), 4.04 (dd, J = 104.4, 11.9 Hz, 1H), 3.66 − 3.55 (m, 2H), 3.46 (p, J = 7.4 Hz, 1H), 3.21 − 3.03 (m, 3H), 2.80 (t, J = 11.9 Hz, 1H), 2.41 (t, J = 12.7 Hz, 1H), 1.57 − 1.53 (m, 1H), 1.33 (t, J = 7.1 Hz, 4H). | 535.25 |
| 1-((3R,4S)-4-(3- ((4-amino-5-(4- chloro-3- methoxyphenyl)- 7-isopropyl-7H- pyrrolo[2,3- d]pyrimidin-6- yl)ethynyl)azetidin- 1-yl)-3- fluoropiperidin-1- yl)prop-2-en-1-one | 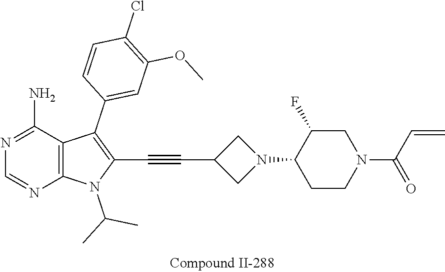 [SMILES: C=CC(=O)N1CC[C@H](N2CC(C#Cc3c(-c4ccc(Cl)c(OC)c4)c4c(N)ncnc4n3C(C)C)C2)[C@H](F)C1] | ¹H NMR (400 MHz, Methanol-d₄) δ 8.17 (s, 1H), 7.52 (d, J = 8.1 Hz, 1H), 7.23 (d, J = 1.9 Hz, 1H), 7.11 (dd, J = 8.1, 1.9 Hz, 1H), 6.78 (ddd, J = 25.4, 16.8, 10.7 Hz, 1H), 6.25 − 6.15 (m, 1H), 5.76 (ddd, J = 10.7, 6.0, 1.9 Hz, 1H), 5.20 (p, J = 6.9 Hz, 1H), 4.77 − 4.52 (m, 2H), 4.39 − 4.05 (m, 1H), 3.96 (s, 3H), 3.74 (dt, J = 14.4, 7.2 Hz, 2H), 3.55 (p, J = 7.8 Hz, 1H), 3.31 − 3.12 (m, 3H), 2.87 (t, J = 12.4 Hz, 1H), 2.54 (d, J = 26.3 Hz, 1H), 1.70 (d, J = 7.0 Hz, 7H), 1.57 (dd, J = 26.8, 13.4 Hz, 1H). | 551.2 |
| 1-((3R,4S)-4-(3- ((4-amino-7-ethyl- 5-(7- fluorobenzo[d][1,3] dioxol-4-yl)-7H- pyrrolo[2,3- d]pyrimidin-6- yl)ethynyl)azetidin- 1-yl)-3- hydroxypiperidin- 1-yl)prop-2-en-1- one | 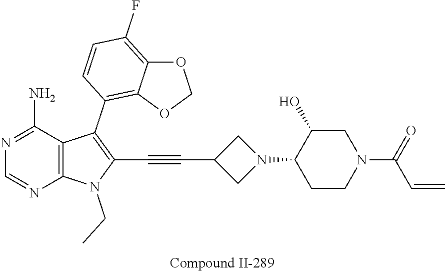 [SMILES: C=CC(=O)N1CC[C@H](N2CC(C#Cc3c(-c4ccc(F)c5c4OCO5)c4c(N)ncnc4n3CC)C2)[C@H](O)C1] | ¹H NMR (400 MHz, DMSO-d₆) δ 8.15 (s, 1H), 6.96 (t, J = 9.4 Hz, 1H), 6.89 (dd, J = 8.8, 5.0 Hz, 1H), 6.76 (ddd, J = 20.9, 16.7, 10.5 Hz, 1H), 6.27 (s, 1H), 6.15 (s, 2H), 6.05 (dt, J = 16.8, 2.4 Hz, 1H), 5.63 (ddd, J = 10.4, 5.3, 2.5 Hz, 1H), 4.54 − 4.42 (d, J = 3.3 Hz, 1H), 4.24 (q, J = 7.9, 7.4 Hz, 2H), 4.00 − 3.80 (dd, J = 13.4, 5.0 Hz, 1H), 3.74 − 3.56 (m, 4H), 3.43 (q, J = 7.6 Hz, 1H), 3.24 − 3.14 (m, 1H), 3.05 (dt, J = 13.8, 6.9 Hz, 2H), 2.90 (dt, J = 13.2, 6.8 Hz, 1H), 2.26 (d, J = 7.1 Hz, 1H), 1.51 − 1.29 (m, 5H). | 533.2 |
| 1-(4-(3-((4-amino- 7-ethyl-5-(7- fluorobenzo[d][1,3] dioxol-4-yl)-7H- pyrrolo[2,3- d]pyrimidin-6- yl)ethynyl)azetidin- 1-yl)piperidin-1- yl)prop-2-en-1-one | 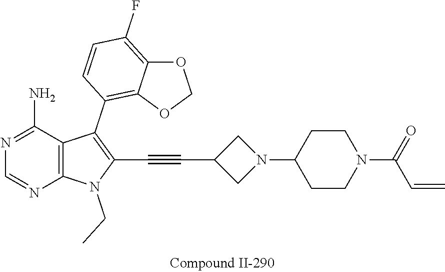 [SMILES: C=CC(=O)N1CCC(N2CC(C#Cc3c(-c4ccc(F)c5c4OCO5)c4c(N)ncnc4n3CC)C2)CC1] | ¹H NMR (400 MHz, DMSO-d₆) δ 8.15 (s, 1H), 6.96 (dd, J = 10.0, 8.8 Hz, 1H), 6.89 (dd, J = 8.8, 5.0 Hz, 1H), 6.79 (dd, J = 16.7, 10.5 Hz, 1H), 6.28 (s, 1H), 6.15 (s, 2H), 6.07 (dd, J = 16.6, 2.5 Hz, 1H), 5.65 (dd, J = 10.4, 2.5 Hz, 1H), 4.24 (q, J = 8.2, 7.6 Hz, 2H), 3.98 (d, J = 13.1 Hz, 1H), 3.82 (d, J = 13.2 Hz, 1H), 3.55 (t, J = 6.9 Hz, 2H), 3.41 (p, J = 7.4 Hz, 1H), 3.19 (t, J = 11.2 Hz, 1H), 2.97 (q, J = 7.1 Hz, 3H), 2.25 (td, J = 8.5, 4.1 Hz, 1H), 1.63 − 1.57 (m, 2H), 1.33 (t, J = 7.1 Hz, 3H), 1.07 (s, 2H). | 517.4 |
| (2E)-1- [(1R,5S,6R)-6-[2- [4-amino-5-(7- fluoro-2H-1,3- benzodioxol-4-yl)- 7-methyl-7H- pyrrolo[2,3- d]pyrimidin-6- ylethynyl]-3- azabicyclo[3.1.0] hexan-3-yl]-4- (dimethylamino) but-2-en-1-one | 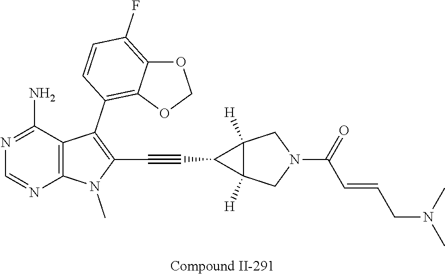 [SMILES: CN(C)C/C=C/C(=O)N1C[C@@H]2[C@@H](C#Cc3c(-c4ccc(F)c5c4OCO5)c4c(N)ncnc4n3C)[C@@H]2C1] | ¹H NMR (400 MHz, DMSO-d₆) δ 8.15 (s, 1H), 6.96 (dd, J = 10.0, 8.8 Hz, 1H), 6.86 (dd, J = 8.8, 4.9 Hz, 1H), 6.62-6.55 (m, J = 15.2, 6.2 Hz, 1H), 6.28 (d, J = 15.1 Hz, 2H), 6.14 (s, 2H), 3.85 (d, J = 10.7 Hz, 1H), 3.75 − 3.72 (m, 1H), 3.68 (s, 3H), 3.62 (dd, J = 10.8, 4.1 Hz, 1H), 3.38 (dd, J = 12.6, 4.3 Hz, 1H), 3.00 (d, J = 6.2 Hz, 2H), 2.14 (s, 6H), 2.06 (m, 1H), 2.01 (m, 1H), 1.48 (t, J = 3.5 Hz, 1H). | 503.30 |
| 1-((3R,4S)-4-(3- ((4-amino-5-(4- chloro-3- methoxyphenyl)- 7-(2-hydroxy-2- methylpropyl)-7H- pyrrolo[2,3- d]pyrimidin-6- yl)ethynyl)azetidin- 1-yl)-3- fluoropiperidin-1- yl)prop-2-en-1-one | 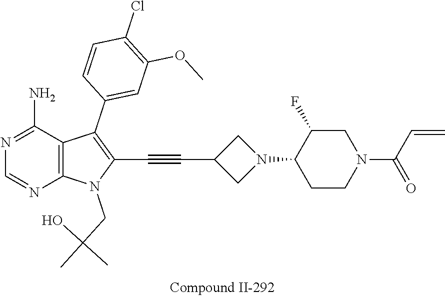 [SMILES: C=CC(=O)N1CC[C@H](N2CC(C#Cc3c(-c4ccc(Cl)c(OC)c4)c4c(N)ncnc4n3CC(C)(C)O)C2)[C@H](F)C1] | ¹H NMR (400 MHz, DMSO-d₆) δ 8.19 (s, 1H), 7.54 (d, J = 8.1 Hz, 1H), 7.23 (d, J = 1.9 Hz, 1H), 7.10 (dd, J = 8.1, 1.9 Hz, 1H), 6.79 (ddd, J = 23.5, 16.6, 10.5 Hz, 1H), 6.44 (br, 1H), 6.09 (dd, J = 16.7, 2.4 Hz, 1H), 5.67 (d, J = 10.1 Hz, 1H), 4.94 (s, 1H), 4.67 (d, J = 48.6 Hz, 1H), 4.06 (d, J = 123.0 Hz, 7H), 3.67 − 3.52 (m, 2H), 3.52 − 3.40 (m, 1H), 3.29 − 3.03 (m, 3H), 2.82 (t, J = 12.1 Hz, 1H), 2.48 − 2.30 (m, 1H), 1.55 (d, J = 12.3 Hz, 1H), 1.46 − 1.25 (m, 1H), 1.16 (s, 6H). | 581.25 |
| (E)-1-((1R,5S,6s)- 6-((4-amino-5-(7- fluorobenzo[d][1,3] dioxol-4-yl)-7- isopropyl-7H- pyrrolo[2,3- d]pyrimidin-6- yl)ethynyl)-3- azabicyclo[3.1.0] hexan-3-yl)-4- (dimethylamino) but-2-en-1-one | 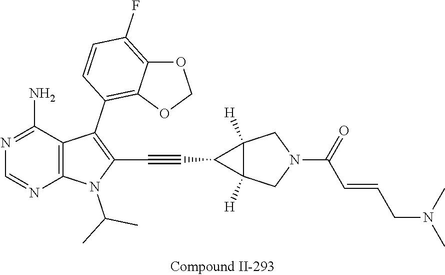 [SMILES: CC(C)n1c(C#C[C@@H]2[C@H]3CN(C(=O)/C=C/CN(C)C)C[C@@H]23)c(-c2ccc(F)c3c2OCO3)c2c(N)ncnc21] | ¹H NMR (400 MHz, DMSO-d₆) δ 8.13 (s, 1H), 6.95 (dd, J = 10.0, 8.8 Hz, 1H), 6.86 (dd, J = 8.9, 5.0 Hz, 1H), 6.58 (dt, J = 15.2, 6.1 Hz, 1H), 6.28 (dt, J = 15.4, 1.6 Hz, 1H), 6.19 (s, 2H), 6.14 (d, J = 10.7 Hz, 2H), 5.04 (p, J = 6.9 Hz, 1H), 3.85 (d, J = 10.8 Hz, 1H), 3.74 (d, J = 12.5 Hz, 1H), 3.62 (dd, J = 10.9, 4.2 Hz, 1H), 3.32 (dd, J = 10.9, 4.2 Hz, 1H), 3.00 (dd, J = 6.2, 1.5 Hz, 2H), 2.13 (s, 6H), 1.96 (s, 1H), 1.58 (d, J = 6.9 Hz, 6H), 1.48 (t, J = 3.4 Hz, 1H). | 531.3 |
| (E)-1-((1R,5S,6s)- 6-((4-amino-7- ethyl-5-(7- fluorobenzo[d][1,3] dioxol-4-yl)-7H- pyrrolo[2,3- d]pyrimidin-6- yl)ethynyl)-3- azabicyclo[3.1.0] hexan-3-yl)-4- (dimethylamino) but-2-en-1-one | 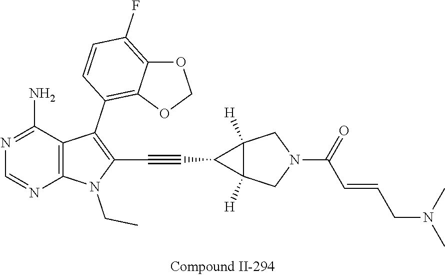 [SMILES: CCn1c(C#C[C@@H]2[C@H]3CN(C(=O)/C=C/CN(C)C)C[C@@H]23)c(-c2ccc(F)c3c2OCO3)c2c(N)ncnc21] | ¹H NMR (400 MHz, DMSO-d₆) δ 8.14 (s, 1H), 6.96 (dd, J = 10.0, 8.8 Hz, 1H), 6.87 (dd, J = 8.9, 5.0 Hz, 1H), 6.58 (dt, J = 15.0, 6.2 Hz, 1H), 6.28 (m, 2H), 6.15 (s, 2H), 4.26 − 4.18 (m, 2H), 3.85 (d, J = 10.8 Hz, 1H), 3.74 (d, J = 12.5 Hz, 1H), 3.62 (dd, J = 10.6, 4.2 Hz, 1H), 3.38 (dd, J = 12.5, 4.3 Hz, 1H), 3.04 − 2.98 (m, 2H), 2.14 (s, 6H), 2.05 (m, 2H), 1.49 (t, J = 3.5 Hz, 1H), 1.30 (t, J = 7.1 Hz, 3H). | 517.30 |
| 1-((3R,4S)-4-(3- ((4-amino-5-(4- bromo-3- methoxyphenyl)- 7-ethyl-7H- pyrrolo[2,3- d]pyrimidin-6- yl)ethynyl)azetidin- 1-yl)-3- hydroxypiperidin- 1-yl)prop-2-en-1- one | 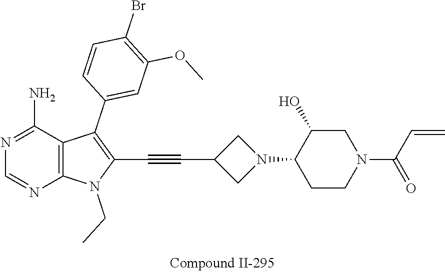 [SMILES: C=CC(=O)N1CC[C@H](N2CC(C#Cc3c(-c4ccc(Br)c(OC)c4)c4c(N)ncnc4n3CC)C2)[C@H](O)C1] | ¹H NMR (400 MHz, DMSO-d₆) δ 8.19 (s, 1H), 7.68 (d, J = 8.1 Hz, 1H), 7.20 (d, J = 1.9 Hz, 1H), 7.04 (dd, J = 8.1, 1.9 Hz, 1H), 6.80 − 6.67 (m, 1H), 6.31 (br, 1H), 6.08 − 6.02 (m, 1H), 5.66 − 5.60 (m, 1H), 4.58 − 4.40 (m, 1H), 4.27 (q, J = 7.0 Hz, 2H), 4.00 (d, J = 12.4 Hz, 1H), 3.90 (s, 3H), 3.79 (d, J = 13.7 Hz, 1H), 3.72 − 3.51 (m, 4H), 3.46 (t, J = 7.5 Hz, 1H), 3.21 (d, J = 13.4 Hz, 1H), 3.08 (q, J = 8.3, 6.9 Hz, 2H), 2.92 (s, 1H), 2.28 (s, 1H), 1.36 (dt, J = 14.2, 5.6 Hz, 5H). | 581.3 |
| 1-((3R,4S)-4-(3- ((4-amino-5-(4- chloro-3- methoxyphenyl)- 7- (cyclopropylmethyl)- 7H-pyrrolo[2,3- d]pyrimidin-6- yl)ethynyl)azetidin- 1-yl)-3- hydroxypiperidin- 1-yl)prop-2-en-1- one | 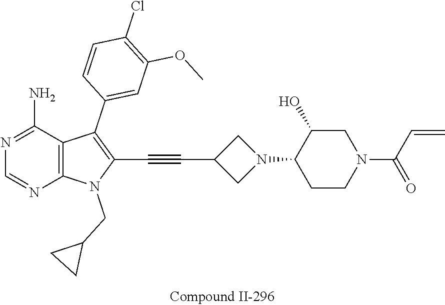 [SMILES: C=CC(=O)N1CC[C@H](N2CC(C#Cc3c(-c4ccc(Cl)c(OC)c4)c4c(N)ncnc4n3CC3CC3)C2)[C@H](O)C1] | ¹H NMR (400 MHz, DMSO-d₆) δ 8.20 (s, 1H), 7.54 (d, J = 8.1 Hz, 1H), 7.23 (d, J = 1.9 Hz, 1H), 7.12 (dd, J = 8.1, 1.9 Hz, 1H), 6.78 (ddd, J = 27.2, 16.7, 10.5 Hz, 1H), 6.33 (br, 1H), 6.07 (dd, J = 16.7, 2.5 Hz, 1H), 5.73 − 5.59 (m, 1H), 4.13 (d, J = 7.1 Hz, 3H), 3.91 (s, 5H), 3.72 (s, 4H), 3.19 (d, J = 13.7 Hz, 2H), 2.78 (s, 1H), 1.54 (s, 1H), 1.47 − 1.20 (m, 2H), 0.49 (ddd, J = 6.3, 4.1, 2.0 Hz, 4H). | 561.25 |
| 1-((3R,4S)-4-(3- ((4-amino-5-(4- chloro-3- ethylphenyl)-7- isopropyl-7H- pyrrolo[2,3- d]pyrimidin-6- yl)ethynyl)azetidin- 1-yl)-3- hydroxypiperidin- 1-yl)prop-2-en-1- one | 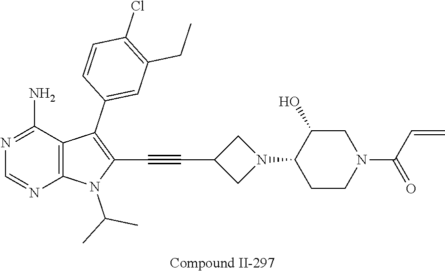 [SMILES: C=CC(=O)N1CC[C@H](N2CC(C#Cc3c(-c4ccc(Cl)c(CC)c4)c4c(N)ncnc4n3C(C)C)C2)[C@H](O)C1] | ¹H NMR (400 MHz, DMSO-d₆) δ 8.17 (s, 1H), 7.56 − 7.45 (m, 2H), 7.34 (dd, J = 8.2, 2.2 Hz, 1H), 6.76 (m, 1H), 6.18 (s, 2H), 6.05 (dd, J = 16.7, 2.7 Hz, 1H), 5.68 − 5.59 (m, 1H), 5.15 − 5.07 (m, 1H), 4.57 − 4.43 (d, 1H), 4.04 − 3.76 (dd, J = 13.5, 5.3 Hz, 1H), 3.74 − 3.51 (m, 4H), 3.41 − 3.38 (m, 1H), 3.22 (d, J = 13.4 Hz, 1H), 3.06 (d, J = 7.2 Hz, 2H), 2.95 (m, 1H),2.81 (q, J = 7.5 Hz, 2H), 2.35 (dd, 1H), 1.62 (d, J = 6.9 Hz,6H), 1.41 − 1.35 (m, 2H), 1.23 (t, J = 7.5 Hz, 3H). | 547.3 |
| 1-((3R,4S)-4-(3- ((4-amino-5-(4- chloro-3- (difluoromethoxy) phenyl)-7- isopropyl-7H- pyrrolo[2,3- d]pyrimidin-6- yl)ethynyl)azetidin- 1-yl)-3- hydroxypiperidin- 1-yl)prop-2-en-1- one | 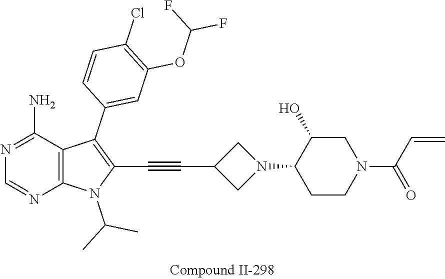 [SMILES: C=CC(=O)N1CC[C@H](N2CC(C#Cc3c(-c4ccc(Cl)c(OC(F)F)c4)c4c(N)ncnc4n3C(C)C)C2)[C@H](O)C1] | ¹H NMR (400 MHz, DMSO-d₆) δ 8.18 (s, 1H), 7.70 (d, J = 8.4 Hz, 1H), 7.67 − 7.53 (m, 1H), 7.53 − 7.13 (m, 3H), 6.76 (ddd, J = 21.1, 16.7, 10.4 Hz, 1H), 6.31 (s, 1H), 6.05 (dd, J = 16.7, 2.5 Hz, 1H), 5.67 − 5.60 (m, 1H), 5.11 (d, J = 7.3 Hz, 1H), 4.49 (d, J = 54.1 Hz, 1H), 3.90 (dd, J = 82.1, 13.1 Hz, 1H), 3.61 (dd, J = 27.9, 8.3 Hz, 4H), 3.21 (d, J = 13.5 Hz, 1H), 3.06 (s, 2H), 2.92 (s, 1H), 2.28 (s, 1H), 1.62 (d, J = 7.0 Hz, 6H), 1.38 (s, 2H). | 585.4 |
| 1-((3R,4S)-4-(3- ((4-amino-5-(4- chloro-3- (methylthio)phenyl)- 7-isopropyl-7H- pyrrolo[2,3- d]pyrimidin-6- yl)ethynyl)azetidin- 1-yl)-3- hydroxypiperidin- 1-yl)prop-2-en-1- one | 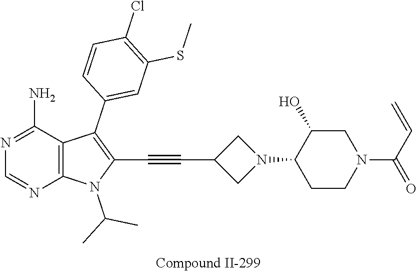 [SMILES: C=CC(=O)N1CC[C@H](N2CC(C#Cc3c(-c4ccc(Cl)c(SC)c4)c4c(N)ncnc4n3C(C)C)C2)[C@H](O)C1] | ¹H NMR (400 MHz, DMSO-d₆) δ 8.18 (s, 1H), 7.55 (d, J = 8.0 Hz, 1H), 7.36 (d, J = 2.0 Hz, 1H), 7.28 (dd, J = 8.2, 1.9 Hz, 1H), 6.76 − 6.70 (m, 1H), 6.24 (br, 1H), 6.05 (d, J = 16.7 Hz, 1H), 5.64-5.61 (m, 1H), 5.12 (d, J = 7.2 Hz, 1H), 4.49 (dd, J = 54.4, 3.3 Hz, 1H), 3.90 (dd, J = 79.9, 11.1 Hz, 1H), 3.62 (dt, J = 25.3, 7.3 Hz, 4H), 3.44 − 3.42 (m, 1H), 3.26 (d, J = 13.6 Hz, 1H), 3.12 − 2.98 (m, 2H), 2.93 (d, J = 7.3 Hz, 1H), 2.53 (s, 3H), 2.27 (s, 1H), 1.63 (d, J = 6.9 Hz, 6H), 1.55 − 1.36 (m, 2H). | 565.3 |
| 4-(6-((1-((3R,4S)- 1-acryloyl-3- hydroxypiperidin- 4-yl)azetidin-3- yl)ethynyl)-4- amino-7- isopropyl-7H- pyrrolo[2,3- d]pyrimidin-5-yl)- 2- methoxybenzonitrile | 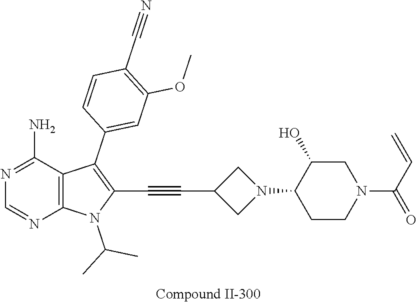 [SMILES: C=CC(=O)N1CC[C@H](N2CC(C#Cc3c(-c4ccc(C#N)c(OC)c4)c4c(N)ncnc4n3C(C)C)C2)[C@H](O)C1] | ¹H NMR (400 MHz, Chloroform-d) δ 8.34 (s, 1H), 7.72 (d, J = 7.8 Hz, 1H), 7.24 (d, J = 8.2 Hz, 1H), 7.15 (s, 1H), 6.64 (dd, J = 16.7, 10.6 Hz, 1H), 6.28 (d, J = 16.9 Hz, 1H), 5.87 (br, 1H), 5.69 (d, J = 11.0 Hz, 1H), 5.22 (p, J = 6.8 Hz, 1H), 4.75 (d, J = 13.4 Hz, 1H), 4.20 (d, 1H) 4.00 (s, 4H), 3.86 (s, 2H), 3.67 (d, J = 8.5 Hz, 1H), 3.42 (t, J = 7.4 Hz, 1H), 3.29 (d, J = 25.8 Hz, 1H), 3.12 (d, J = 14.4 Hz, 1H), 2.58 (d, J = 18.6 Hz, 2H), 1.73 (d, J = 6.9 Hz, 6H), 1.62 (d, J = 16.7 Hz, 2H). | 540.30 |
| 1-((3R,4S)-4-(3- ((4-amino-5-(4- chloro-3- (methylsulfonyl) phenyl)-7- isopropyl-7H- pyrrolo[2,3- d]pyrimidin-6- yl)ethynyl)azetidin- 1-yl)-3- hydroxypiperidin- 1-yl)prop-2-en-1- one | 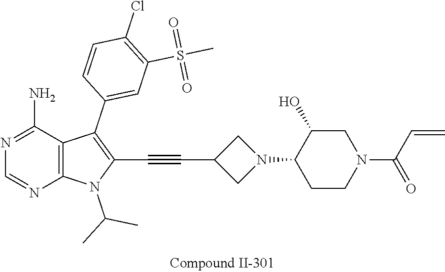 [SMILES: C=CC(=O)N1CC[C@H](N2CC(C#Cc3c(-c4ccc(Cl)c(S(C)(=O)=O)c4)c4c(N)ncnc4n3C(C)C)C2)[C@H](O)C1] | ¹H NMR (400 MHz, DMSO-d₆) δ 8.19 (d, J = 2.0 Hz, 2H), 7.84 (d, J = 8.6 Hz, 1H), 7.78 (dd, J = 8.3, 2.2 Hz, 1H), 6.76 (ddd, J = 22.2, 16.7, 10.5 Hz, 1H), 6.49 (br, 2H), 6.05 (dd, J = 16.8, 2.5 Hz, 1H), 5.63 (dt, J = 10.3, 3.3 Hz, 1H), 5.20 − 5.05 (m, 1H), 4.45 (dd, J = 51.2, 3.4 Hz, 1H), 4.11 − 3.78 (m, 1H), 3.78 − 3.50 (m, 4H), 3.46 (q, J = 7.7 Hz, 4H), 3.20 (d, J = 13.3 Hz, 1H), 3.17 − 3.04 (m, 2H), 2.89 (dd, J = 13.4, 6.7 Hz, 1H), 2.35 (d, J = 30.0 Hz, 1H), 1.63 (d, J = 6.9 Hz, 6H), 1.37 (d, J = 5.5 Hz, 2H). | 597.20 |
| 1-[4-[2-(4-amino- 5-[3-methoxy-4- [(6-methylpyridin- 2-yl)oxy]phenyl]- 7-(propan-2-yl)- 7H-pyrrolo[2,3- d]pyrimidin-6- yl)ethynyl]piperidin- 1-yl]prop-2-en- 1-one | 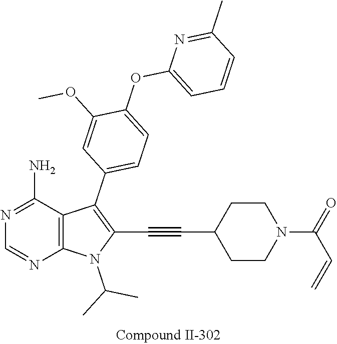 [SMILES: C=CC(=O)N1CCC(C#Cc2c(-c3ccc(Oc4cccc(C)n4)c(OC)c3)c3c(N)ncnc3n2C(C)C)CC1] | ¹H NMR (400 MHz, DMSO-d₆) δ 8.18 (s, 1H), 7.68 (dd, J = 8.3, 7.3 Hz, 1H), 7.24 (d, J = 2.0 Hz, 1H), 7.21 (d, J = 8.1 Hz, 1H), 7.10 (dd, J = 8.1, 1.9 Hz, 1H), 6.95 (d, J = 7.3 Hz, 1H), 6.83 − 6.67 (m, 2H), 6.45 − 6.15 (br, 1H), 6.07 (dd, J = 16.7, 2.4 Hz, 1H), 5.64 (dd, J = 10.5, 2.4 Hz, 1H), 5.14 (p, J = 6.9 Hz, 1H), 3.81 (dd, 1H), 3.73 (s, 4H), 3.40 (t, J = 11.2 Hz, 1H), 3.28 (t, 1H), 3.06 (tt, J = 8.3, 4.0 Hz, 1H), 2.31 (s, 3H), 1.84 (m, 1H), 1.64 (s, 3H), 1.63 (s, 3H), 1.54 (t, 1H). | 551.35 |
| 1-(4-((4-amino-5- (3-methoxy-4-((6- methylpyridin-2- yl)oxy)phenyl)-7- methyl-7H- pyrrolo[2,3- d]pyrimidin-6- yl)ethynyl)piperidin- 1-yl)prop-2-en- 1-one | 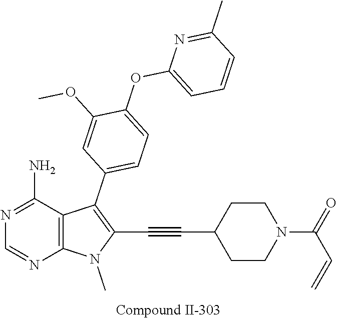 [SMILES: C=CC(=O)N1CCC(C#Cc2c(-c3ccc(Oc4cccc(C)n4)c(OC)c3)c3c(N)ncnc3n2C)CC1] | ¹H NMR (400 MHz, DMSO-d₆) 8.20 (s, 1H), 7.68 (t, J = 7.8 Hz, 1H), 7.27 − 7.19 (m, 2H), 7.12 (dd, J = 8.1, 2.0 Hz, 1H), 6.95 (d, J = 7.3 Hz, 1H), 6.78 (dd, J = 16.7, 10.5 Hz, 1H), 6.70 (d, J = 8.2 Hz, 1H), 6.25 (s, 2H), 6.08 (dd, J = 16.7, 2.4 Hz, 1H), 5.65 (dd, J = 10.5, 2.4 Hz, 1H), 3.74 (m, 8H), 3.44 (s, 2H), 3.11 − 3.03 (m, 1H), 2.32 (s, 3H), 1.85 (s, 2H), 1.58 (s, 2H). | 523.3 |
| 1-(4-((4-amino-5- (3-methoxy-4-((6- methylpyridin-2- yl)oxy)phenyl)-7- methyl-7H- pyrrolo[2,3- d]pyrimidin-6- yl)ethynyl)-4- hydroxypiperidin- 1-yl)prop-2-en-1- one | 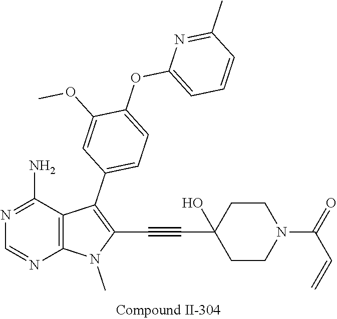 [SMILES: C=CC(=O)N1CCC(O)(C#Cc2c(-c3ccc(Oc4cccc(C)n4)c(OC)c3)c3c(N)ncnc3n2C)CC1] | ¹H NMR (400 MHz, DMSO-d₆) δ 8.22 (s, 1H), 7.68 (dd, J = 8.2, 7.3 Hz, 1H), 7.27 (d, J = 2.0 Hz, 1H), 7.22 (d, J = 8.1 Hz, 1H), 7.12 (dd, J = 8.1, 1.9 Hz, 1H), 6.95 (d, J = 7.3 Hz, 1H), 6.78 (dd, J = 16.7, 10.5 Hz, 1H), 6.70 (d, J = 8.2 Hz, 1H), 6.25 (s, 2H), 6.07 (dd, J = 16.7, 2.4 Hz, 1H), 5.95 (s, 1H), 5.64 (dd, J = 10.5, 2.4 Hz, 1H), 3.86 (s, 1H), 3.76 (d, J = 9.8 Hz, 7H), 3.42 (t, J = 11.4 Hz, 1H), 3.22 (m, 1H), 2.32 (s, 3H), 1.87 (s, 2H), 1.67 (d, J = 12.2 Hz, 2H). | 539.2 |
| 1-(4-(3-((4-amino- 5-(3-methoxy-4- (6-methylpyridin- 2-yloxy)phenyl)-7- methyl-7H- pyrrolo[2,3- d]pyrimidin-6- yl)ethynyl)-3- methylazetidin-1- yl)piperidin-1- yl)prop-2-en-1-one formate | 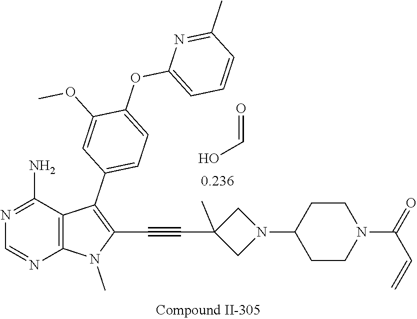 | ¹H NMR (400 MHz, Chloroform-d) δ 8.30 (s, 1H), 7.60 (t, J = 7.7 Hz, 1H), 7.25 (d, J = 8.1 Hz, 1H), 7.19 (d, J = 1.9 Hz, 1H), 7.13 (dd, J = 8.1, 2.0 Hz, 1H), 6.90 (d, J = 7.4 Hz, 1H), 6.69 (d, J = 8.2 Hz, 1H), 6.57 (dd, J = 16.9, 10.6 Hz, 1H), 6.26 (dd, J = 16.8, 1.9 Hz, 1H), 5.69 (dd, J = 10.6, 1.9 Hz, 1H), 4.30 (d, J = 13.1 Hz, 1H), 3.88 (s, 4H), 3.84 (s, 3H), 3.39 (s, 4H), 3.16 (t, J = 11.8 Hz, 1H), 3.06 − 2.96 (m, 1H), 2.45-2.42 (m, 4H), 1.71 (d, J = 12.7 Hz, 2H), 1.64 (s, 3H), 1.33 (d, J = 10.6 Hz, 2H). | 592.25 |
| 1-[4-[2-(4-amino- 5-[3-methoxy-4- [(6-methylpyridin- 2-yl)oxy]phenyl]- 7-(propan-2-yl)- 7H-pyrrolo[2,3- d]pyrimidin-6- yl)ethynyl]-4- methylpiperidin-1- yl]prop-2-en-1-one | 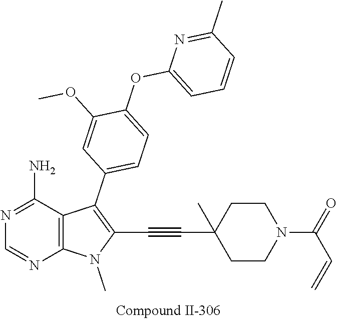 [SMILES: C=CC(=O)N1CCC(C)(C#Cc2c(-c3ccc(Oc4cccc(C)n4)c(OC)c3)c3c(N)ncnc3n2C)CC1] | ¹H NMR (400 MHz, DMSO-d₆) δ 8.18 (s, 1H), 7.67 (t, 1H), 7.29 − 7.16 (m, 2H), 7.12 (dd, J = 8.1, 1.9 Hz, 1H), 6.94 (d, J = 7.3 Hz, 1H), 6.73 (dd, J = 16.7, 10.5 Hz, 1H), 6.65 (d, J = 8.2 Hz, 1H), 6.40 − 6.12 (br, 1H), 6.06 (dd, J = 16.7, 2.4 Hz, 1H), 5.62 (dd, J = 10.5, 2.4 Hz, 1H), 5.31 − 4.92 (m, 1H), 4.35 (d, J = 13.4 Hz, 1H), 3.97 (d, J = 13.8 Hz, 1H), 3.72 (s, 3H), 3.16 (t, J = 13.1 Hz, 1H), 2.79 (t, J = 12.8 Hz, 1H), 2.31 (s, 3H), 1.74 (d, J = 13.1 Hz, 2H), 1.65 (s, 3H), 1.64 (s, 3H), 1.42 (q, J = 13.0 Hz, 2H), 1.30 (s, 3H). | 565.40 |
| 1-(4-(3-((4-amino- 7-isopropyl-5-(3- methoxy-4-(6- methylpyridin-2- yloxy)phenyl)-7H- pyrrolo[2,3- d]pyrimidin-6- yl)ethynyl)azetidin- 1-yl)piperidin-1- yl)prop-2-en-1-one | 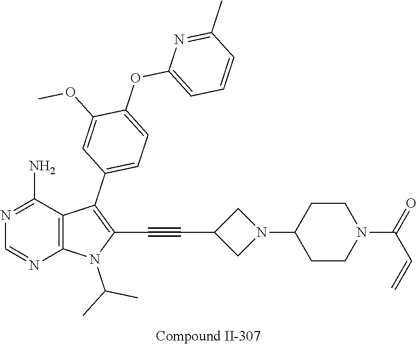 [SMILES: C=CC(=O)N1CCC(N2CC(C#Cc3c(-c4ccc(Oc5cccc(C)n5)c(OC)c4)c4c(N)ncnc4n3C(C)C)C2)CC1] | ¹H NMR (400 MHz, DMSO-d₆) δ 8.18 (s, 1H), 7.69 (t, J = 7.8 Hz, 1H), 7.26 − 7.22 (m, 2H), 7.11 (dd, J = 8.1, 1.9 Hz, 1H), 6.95 (d, J = 7.3 Hz, 1H), 6.84 − 6.68 (m, 2H), 6.27 (s, 1H), 6.06 (dd, J = 16.7, 2.5 Hz, 1H), 5.64 (dd, J = 10.4, 2.5 Hz, 1H), 5.12 (hept, J = 6.9 Hz, 1H), 3.95 (d, J = 12.4 Hz, 1H), 3.76 (s, 4H), 3.55 (t, J = 6.8 Hz, 2H), 3.45 (p, J = 7.2 Hz, 1H), 3.21 − 3.10 (m, 1H), 3.07 − 2.89 (m, 3H), 2.32 (s, 3H), 2.22 (tt, J = 8.6, 3.7 Hz, 1H), 1.65-1.56 (d, J = 6.9 Hz, 8H), 1.06 (d, J = 12.9 Hz, 2H). | 606.30 |
| 1-((2S)-4-(3-((4- amino-5-(3- methoxy-4-(6- methylpyridin-2- yloxy)phenyl)-7- methyl-7H- pyrrolo[2,3- d]pyrimidin-6- yl)ethynyl)-3- fluoroazetidin-1- yl)-2- (hydroxymethyl) piperidin-1-yl)prop- 2-en-1-one | 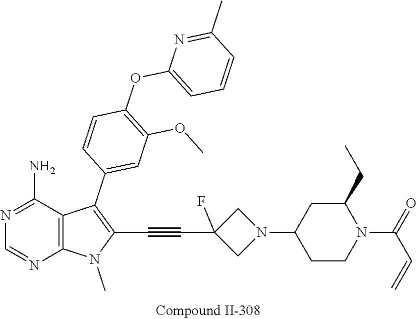 [SMILES: C=CC(=O)N1CCC(N2CC(F)(C#Cc3c(-c4ccc(Oc5cccc(C)n5)c(OC)c4)c4c(N)ncnc4n3C)C2)C[C@H]1CC] | ¹H NMR (400 MHz, Chloroform-d) δ 8.38 (s, 1H), 7.60 (t, J = 7.7 Hz, 1H), 7.27 − 7.16 (m, 3H), 6.90 (d, J = 7.3 Hz, 1H), 6.71 (d, J = 8.1 Hz, 1H), 6.58 (dd, J = 16.8, 10.6 Hz, 1H), 6.26 (d, J = 16.8 Hz, 1H), 5.87 − 5.62 (m, 3H), 4.69 (s, 1H), 3.99 − 3.50 (m, 14H), 2.73 − 2.62 (m, 1H), 2.45 (s, 3H), 1.93 (d, J = 14.2 Hz, 2H), 1.75 − 1.58 (m, 3H). | 626.20 |
| 1-((3R,4S)-4-(3- ((4-amino-5-(3- methoxy-4-((6- methylpyridin-2- yl)oxy)phenyl)-7- methyl-7H- pyrrolo[2,3- d]pyrimidin-6- yl)ethynyl)azetidin- 1-yl)-3- fluoropiperidin-1- yl)prop-2-en-1-one | 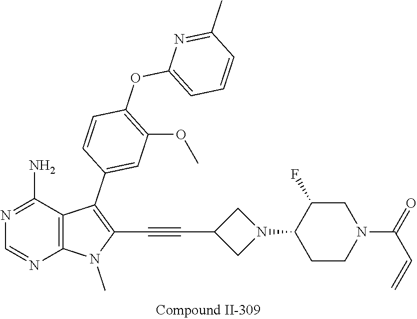 [SMILES: C=CC(=O)N1CC[C@H](N2CC(C#Cc3c(-c4ccc(Oc5cccc(C)n5)c(OC)c4)c4c(N)ncnc4n3C)C2)[C@H](F)C1] | ¹H NMR (400 MHz, DMSO-d₆) δ 8.21 (s, 1H), 7.68 (t, J = 7.8 Hz, 1H), 7.29 − 7.21 (m, 2H), 7.12 (dd, J = 8.1, 1.9 Hz, 1H), 6.96 (d, J = 7.3 Hz, 1H), 6.86 − 6.69 (m, 2H), 6.33 (br, 2H), 6.08 (dd, J = 16.7, 2.4 Hz, 1H), 5.67 (d, J = 9.9 Hz, 1H), 4.71 (s, 1H), 4.59-3.89 (m, 2H), 3.75 (d, J = 4.0 Hz, 6H), 3.61 −3.54 ( m, 3H), 3.26-2.76 (m, 4H), 2.47 (s, 1H), 2.33 (s, 3H), 1.52 (s, 1H), 1.41- 1.29 (m, 1H). | 596.45 |
| N-(2-((1R,5S,6s)- 6-((4-amino-5-(3- methoxy-4-(6- methylpyridin-2- yloxy)phenyl)-7- methyl-7H- pyrrolo[2,3- d]pyrimidin-6- yl)ethynyl)-3-aza- bicyclo[3.1.0] hexan-3-yl)-2- oxoethyl)acrylamide | 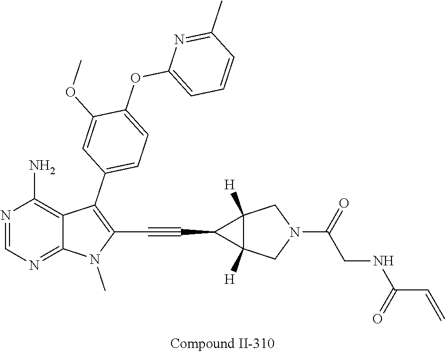 [SMILES: C=CC(=O)NCC(=O)N1C[C@@H]2[C@@H](C#Cc3c(-c4ccc(Oc5cccc(C)n5)c(OC)c4)c4c(N)ncnc4n3C)[C@@H]2C1] | ¹H NMR (400 MHz, DMSO-d₆) δ 8.23 (t, J = 5.5 Hz, 1H), 8.20 (s, 1H), 7.69 (dd, J = 8.2, 7.3 Hz, 1H), 7.28 − 7.21 (m, 2H), 7.09 (dd, J = 8.1, 2.0 Hz, 1H), 6.96 (d, J = 7.3 Hz, 1H), 6.74 (d, J = 8.2 Hz, 1H), 6.38 (dd, J = 17.1, 10.2 Hz, 2H), 6.09 (dd, J = 17.1, 2.2 Hz, 1H), 5.60 (dd, J = 10.2, 2.2 Hz, 1H), 3.98 − 3.82 (m, 2H), 3.73 (dd, J = 13.3, 8.5 Hz, 8H), 3.59 (dd, J = 10.6, 4.2 Hz, 1H), 3.32 (s, 1H), 2.33 (s, 3H), 2.13 (dt, J = 7.4, 3.7 Hz, 1H), 2.04 (dt, J = 7.4, 3.8 Hz, 1H), 1.51 (t, J = 3.4 Hz, 1H). | 578.25 |
| (R)-N-(1-(4-((4- amino-5-(3- methoxy-4-((6- methylpyridin-2- yl)oxy)phenyl)-7- methyl-7H- pyrrolo[2,3- d]pyrimidin-6- yl)ethynyl)piperidin- 1-yl)-3-hydroxy- 1-oxopropan-2- yl)acrylamide | 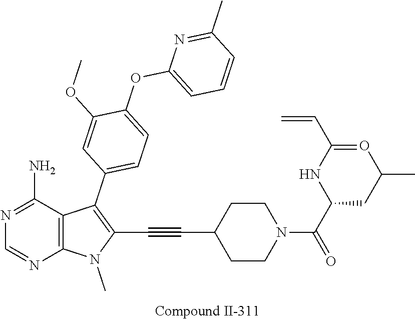 | ¹H NMR (400 MHz, DMSO-d₆) δ 8.34 (d, J = 8.3 Hz, 1H), 8.20 (s, 1H), 7.68 (t, J = 8.0 Hz, 1H), 7.27 − 7.20 (m, 2H), 7.12 (d, J = 8.1 Hz, 1H), 6.95 (dd, J = 7.6, 2.6 Hz, 1H), 6.71 (d, J = 8.2 Hz, 1H), 6.35 (ddd, J = 15.3, 10.0, 4.2 Hz, 1H), 6.25 (s, 2H), 6.09 (dd, J = 17.0, 4.9 Hz, 1H), 5.58 (t, J = 10.4 Hz, 1H), 4.90 (q, J = 8.0, 7.2 Hz, 2H), 3.74 (m, 5H), 3.58 (dt, J = 12.1, 6.1 Hz, 1H), 3.45 (dt, J = 11.1, 6.0 Hz, 2H), 3.35 (m, 4H), 3.07 (s, 1H), 2.32 (s, 3H), 1.86 (s, 2H), 1.66 − 1.56 (m, 1H), 1.53 (d, J = 12.6 Hz, 1H). | 610.3 |
| N-[(1r,4r)-4-[2-[4- amino-7-(2- hydroxyethyl)-5- [3-methoxy-4-[(6- methylpyridin-2- yl)oxy]phenyl]- 7H-pyrrolo[2,3- d]pyrimidin-6- yllethynyl] cyclohexyl] prop-2- enamide | 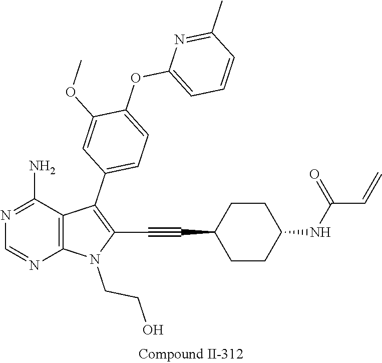 [SMILES: C=CC(=O)N[C@H]1CC[C@H](C#Cc2c(-c3ccc(Oc4cccc(C)n4)c(OC)c3)c3c(N)ncnc3n2CCO)CC1] | ¹H NMR (400 MHz, DMSO-d₆) δ 8.19 (s, 1H), 7.99 (d, J = 7.6 Hz, 1H), 7.69 (t, J = 7.8 Hz, 1H), 7.23 (d, J = 8.0 Hz, 2H), 7.11 (d, J = 8.1 Hz, 1H), 6.95 (d, J = 7.3 Hz, 1H), 6.72 (d, J = 8.3 Hz, 1H), 6.45 − 6.25 (br, 1H), 6.21 (dd, J = 16.8, 10.0 Hz, 1H), 6.06 (dd, J = 17.4, 2.1 Hz, 1H), 5.56 (d, J = 9.9 Hz, 1H), 4.97 (t, J = 5.8 Hz, 1H), 4.29 (t, 2H), 3.75 (s, 5H), 3.62 (m, 1H), 2.62 (m, 1H), 2.32 (s, 3H), 1.99 (d, J = 12.5 Hz, 2H), 1.83 (d, J = 10.7 Hz, 2H), 1.49 (q, 2H), 1.26 (q, J = 12.1 Hz, 2H). | 567.40 |

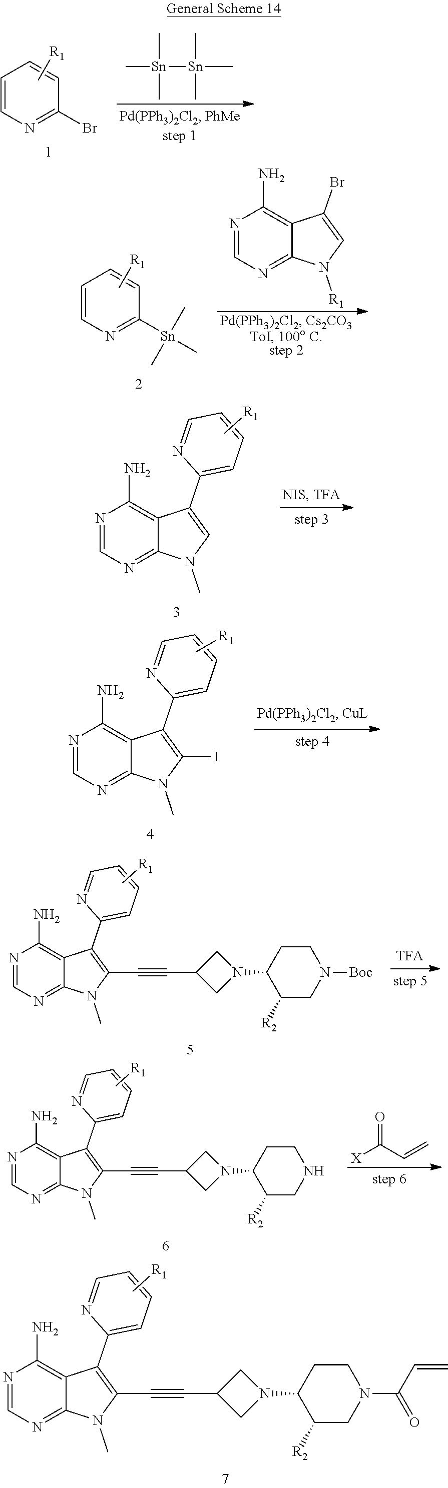

### Example 26

### 1-((3S,4R)-4-(3-((4-amino-5-(5-chloro-6-methoxypyridin-2-yl)-7-methyl-7H-pyrrolo[2,3-d]pyrimidin-6-yl)ethynyl)azetidin-1-yl)-3-hydroxypiperidin-1-yl)prop-2-en-1-one

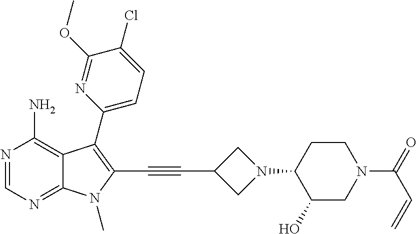 [SMILES: C=CC(=O)N1CC[C@@H](N2CC(C#Cc3c(-c4ccc(Cl)c(OC)n4)c4c(N)ncnc4n3C)C2)[C@@H](O)C1]

### 6-bromo-3-chloro-2-methoxypyridine

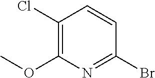

### Step 1

A round bottomed flask was charged with 6-bromo-2-methoxypyridin-3-amine (3.2 g, 15.7 mmol) and HCl (10.5 mL) at 0° C. A solution of sodium nitrite (1.90 g, 27.6 mmol) and water (3 mL) were added into the mixture and stirred 10 min at 0° C., then the mixture of chlorocopper (4.06 g, 41.1 mmol) and HCl (4.5 mL) were dropwise added into the reaction and stirred 75 min at 65° C. The reaction mixture was diluted with water (100 mL), and the aqueous phase was extracted with ethylacetate (60 mL) three times. The combined organic layers dried over sodium sulfate, filtered, and concentrated in vacuo. The resulting crude material was purified by silica gel chromatography (500 g column; eluting with hexanes/ethyl acetate; 10/1). Concentration in vacuo resulted in 6-bromo-3-chloro-2-methoxypyridine (3.05 g, 13.7 mmol).

### 3-chloro-2-methoxy-6-(trimethylstannyl)pyridine

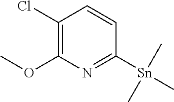

### Step 2

A resealable reaction vial was charged with 6-bromo-3-chloro-2-methoxypyridine (3.5 g, 15.7 mmol), hexamethyldistannane (6.68 g, 20.4 mmol), Pd(PPh₃)₂Cl₂ (1.10 g, 1.57 mmol), and purged with nitrogen three times. Toluene (25 mL) was added, and the mixture was stirred 2 h at 100° C. LCMS indicated conversion to product and the reaction mixture was used in the next step without further purification.

### 5-(5-chloro-6-methoxypyridin-2-yl)-7-methyl-7H-pyrrolo[2,3-d]pyrimidin-4-amine

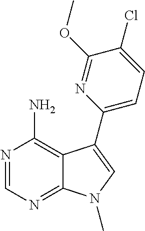 [SMILES: COc1nc(-c2cn(C)c3ncnc(N)c23)ccc1Cl]

### Step 3

A resealable reaction vial was charged with 5-bromo-7-methyl-7H-pyrrolo[2,3-d]pyrimidin-4-amine (3 g, 13.2 mmol), 3-chloro-2-methoxy-6-(trimethylstannyl)pyridine (4.84 g, 15.8 mmol), Pd(PPh₃)₂Cl₂ (926 mg, 1.32 mmol), CS₂CO₃ (12.9 g, 39.5 mmol), and purged with nitrogen three times. Toluene (50 mL) was added, and the mixture was stirred 18 h at 100° C. The reaction mixture was diluted with H₂O (400 mL), and the aqueous phase was extracted with DCM (400 mL) three times. The combined organic layers were dried over sodium sulfate, filtered, and concentrated in vacuo. The resulting crude material was purified by silica gel chromatography (300 g column; eluting with dichloromethane/methanol; 100:1). Concentration in vacuo resulted in 5-(5-chloro-6-methoxypyridin-2-yl)-7-methyl-7H-pyrrolo[2,3-d]pyrimidin-4-amine (1.20 g, 4.14 mmol).

The remaining steps were carried out according to procedures described herein.

Compounds prepared by substantially the same methods, and their characterization data, are listed in Table 17, below.

TABLE 17
Selected Compounds

| | | | MS |
|---|---|---|---|
| Compound | Structure | Proton NMR | [M + 1] |
| 1-(4-(3-((4-amino-7- methyl-5-(5- phenoxypyridin-2-yl)- 7H-pyrrolo[2,3- d]pyrimidin-6- yl)ethynyl)azetidin-1- yl)piperidin-1-yl)prop- 2-en-1-one | 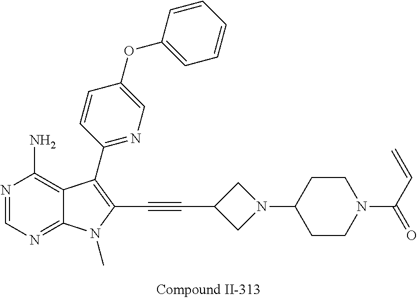 [SMILES: C=CC(=O)N1CCC(N2CC(C#Cc3c(-c4ccc(Oc5ccccc5)cn4)c4c(N)ncnc4n3C)C2)CC1] | ¹H NMR (400 MHz, Methanol- d₄) δ 8.48 (d, J = 8.9 Hz, 1H), 8.40 (d, J = 2.9 Hz, 1H), 8.14 (s, 1H), 7.58-7.33 (m, 3H), 7.24 (t, J = 7.4 Hz, 1H), 7.15 (d, J = 8.0 Hz, 2H), 6.77 (dd, J = 16.8, 10.6 Hz, 1H), 6.19 (d, J = 16.7 Hz, 1H), 5.78-5.72 (m, 1H), 4.39 (d, J = 13.3 Hz, 1H), 4.06 (d, J = 13.6 Hz, 1H), 3.67 (dq, J = 16.4, 8.6, 8.0 Hz, 1H), 3.59-3.38 (m, 2H), 3.19 (t, J = 12.8 Hz, 1H), 2.91 (t, J = 12.3 Hz, 1H), 2.71- 2.48 (m, 1H), 2.10-1.75 (m, 3H), 1.21 (t, J = 11.3 Hz, 2H). | 534.4 |
| 1-(4-(3-((4-amino-5- (6-chloro-5- phenoxypyridin-2-yl)- 7-methyl-7H- pyrrolo[2,3- d]pyrimidin-6- yl)ethynyl)azetidin-1- yl)piperidin-1-yl)prop- 2-en-1-one | 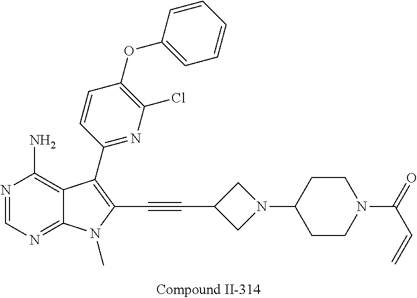 [SMILES: C=CC(=O)N1CCC(N2CC(C#Cc3c(-c4ccc(Oc5ccccc5)c(Cl)n4)c4c(N)ncnc4n3C)C2)CC1] | ¹H NMR (400 MHz, DMSO-d₆) δ 8.40 (d, J = 8.7 Hz, 1H), 8.17 (s, 1H), 7.66 (d, J = 8.6 Hz, 1H), 7.46 (t, J = 7.8 Hz, 2H), 7.24 (t, J = 7.4 Hz, 1H), 7.16 (d, J = 8.0 Hz, 2H), 6.78 (dd, J = 16.7, 10.5 Hz, 1H), 6.12-6.03 (m, 1H), 5.69-5.61 (m, 1H), 4.01 (s, 1H), 3.80 (s, 1H), 3.74 (s, 3H), 3.58 (s, 3H), 3.15 (s, 3H), 2.95 (s, 1H), 2.28 (s, 1H), 1.60 (s, 2H), 1.05 (s, 2H). | 568.4 |
| 1-(4-(3-((4-amino-5- (6-methoxy-5- phenoxypyridin-2-yl)- 7-methyl-7H- pyrrolo[2,3- d]pyrimidin-6- yl)ethynyl)azetidin-1- yl)piperidin-1-yl)prop- 2-en-1-one | 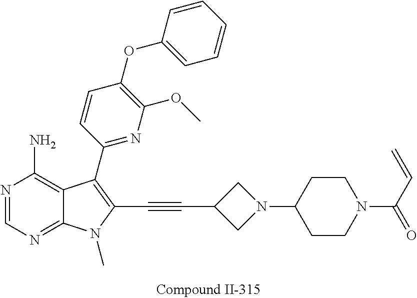 [SMILES: C=CC(=O)N1CCC(N2CC(C#Cc3c(-c4ccc(Oc5ccccc5)c(OC)n4)c4c(N)ncnc4n3C)C2)CC1] | ¹H NMR (400 MHz, Chloroform-d) δ 8.35 (s, 1H), 7.77 (d, J = 8.2 Hz, 1H), 7.39 (t, J = 7.7 Hz, 2H), 7.28 (d, J = 8.7 Hz, 1H), 7.17 (t, J = 7.4 Hz, 1H), 7.07 (d, J = 8.0 Hz, 2H), 6.58 (dd, J = 16.8, 10.6 Hz, 1H), 6.27 (d, J = 16.8 Hz, 1H), 5.69 (d, J = 10.6 Hz, 1H), 4.24 (d, J = 12.9 Hz, 1H), 4.07 (s, 3H), 3.85 (M, 4H), 3.72 (t, J = 7.0 Hz, 2H), 3.54 (p, J = 7.7 Hz, 1H), 3.15 (t, J = 7.0 Hz, 3H), 3.08 (s, 1H), 2.32 (s, 1H), 1.99 (s, 2H), 1.71 (d, J = 13.1 Hz, 2H), 1.29 (d, J = 10.0 Hz, 2H). | 564.3 |
| 1-((3S,4R)-4-(3-((4- amino-5-(6-methoxy- 5-phenoxypyridin-2- yl)-7-methyl-7H- pyrrolo[2,3- d]pyrimidin-6- yl)ethynyl)azetidin-1- yl)-3- hydroxypiperidin-1- yl)prop-2-en-1-one | 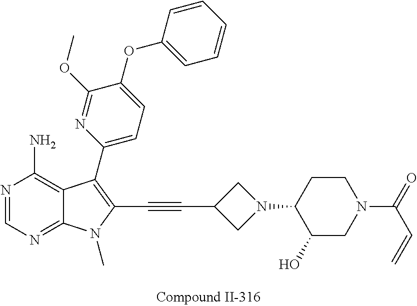 [SMILES: C=CC(=O)N1CC[C@@H](N2CC(C#Cc3c(-c4ccc(Oc5ccccc5)c(OC)n4)c4c(N)ncnc4n3C)C2)[C@@H](O)C1] | ¹H NMR (400 MHz, DMSO-d₆) δ 8.17 (s, 1H), 7.85-7.79 (m, 1H), 7.56 (d, J = 8.2 Hz, 1H), 7.43-7.35 (m, 2H), 7.13 (t, J = 7.4 Hz, 1H), 7.06-6.99 (m, 2H), 6.74 (td, J = 17.1, 10.5 Hz, 1H), 6.04 (dd, J = 16.8, 2.4 Hz, 1H), 5.62 (dd, J = 10.5, 2.5 Hz, 0H), 4.47 (dd, J = 49.8, 3.4 Hz, 1H), 4.01 (d, J = 13.2 Hz, 1H), 3.93 (s, 3H), 3.85-3.66 (m, 5H), 3.66-3.50 (m, 3H), 3.24- 3.13 (m, 3H), 2.85 (s, 1H), 2.34 (d, J = 33.1 Hz, 1H), 1.44 (d, J = 55.8 Hz, 2H). | 580.4 |
| 1-(4-(3-((4-amino-5- (6-methoxy-5- phenoxypyridin-2-yl)- 7-methyl-7H- pyrrolo[2,3- d]pyrimidin-6- yl)ethynyl)-3- fluoroazetidin-1- yl)piperidin-1-yl)prop- 2-en-1-one | 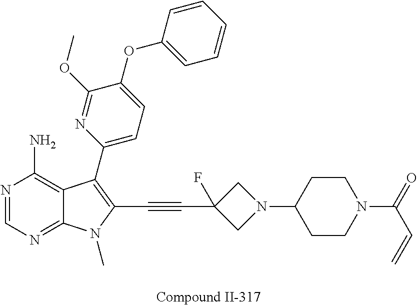 [SMILES: C=CC(=O)N1CCC(N2CC(F)(C#Cc3c(-c4ccc(Oc5ccccc5)c(OC)n4)c4c(N)ncnc4n3C)C2)CC1] | ¹H NMR (400 MHz, DMSO-d₆) δ 8.21 (s, 1H), 7.70 (d, J = 8.1 Hz, 1H), 7.52 (d, J = 8.1 Hz, 1H), 7.39 (t, J = 7.8 Hz, 2H), 7.14 (t, J = 7.4 Hz, 1H), 7.04 (d, J = 8.0 Hz, 2H), 6.77 (dd, J = 16.7, 10.5 Hz, 1H), 6.07 (dd, J = 16.8, 2.5 Hz, 1H), 5.65 (dd, J = 10.5, 2.4 Hz, 1H), 4.03 (d, J = 13.0 Hz, 1H), 3.95 (s, 3H), 3.84 (d, J = 13.6 Hz, 1H), 3.80-3.68 (m, 5H), 3.55 (dd, J = 20.0, 8.8 Hz, 2H), 3.14 (t, J = 11.8 Hz, 1H), 2.93 (t, J = 11.0 Hz, 1H), 2.41 (dt, J = 9.4, 5.1 Hz, 1H), 1.64 (d, J = 12.7 Hz, 2H), 1.08 (s, 2H). | 582.30 |
| 1-((3S,4R)-4-(3-((4- amino-5-(5-chloro-6- methoxypyridin-2-yl)- 7-methyl-7H- pyrrolo[2,3- d]pyrimidin-6- yl)ethynyl)azetidin-1- yl)-3- hydroxypiperidin-1- yl)prop-2-en-1-one | 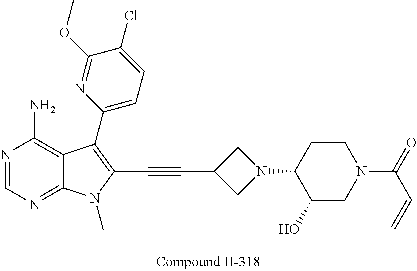 [SMILES: C=CC(=O)N1CC[C@@H](N2CC(C#Cc3c(-c4ccc(Cl)c(OC)n4)c4c(N)ncnc4n3C)C2)[C@@H](O)C1] | ¹H NMR (400 MHz, DMSO-d₆) δ 8.26 (s, 1H), 8.12-8.02 (m, 1H), 7.91-7.87 (m, 1H), 7.58 (s, 2H), 6.74 (d, J = 8.0 Hz, 1H), 6.05 (d, J = 16.6 Hz, 1H), 5.63 (d, J = 8.0 Hz, 1H), 4.58-4.20 (m, 1H), 4.02-3.91 (m, 4H), 3.73-3.62 (m, 9H), 3.19-3.00 (m, 2H), 2.92-2.79 (m, 1H), 2.33- 2.16 (m, 1H), 1.53-1.23 (m, 2H). | 522.30 |
| 1-((2S)-4-(3-((4- amino-5-(5-chloro-6- methoxypyridin-2-yl)- 7-methyl-7H- pyrrolo[2,3- d]pyrimidin-6- yl)ethynyl)azetidin-1- yl)-2- (hydroxymethyl) piperidin-1-yl)prop- 2-en-1-one | 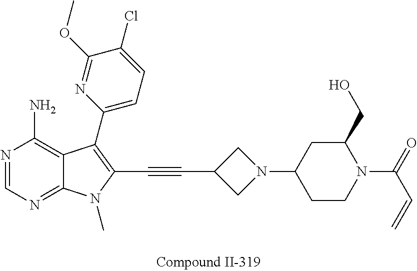 [SMILES: C=CC(=O)N1CCC(N2CC(C#Cc3c(-c4ccc(Cl)c(OC)n4)c4c(N)ncnc4n3C)C2)C[C@H]1CO] | ¹H NMR (400 MHz, DMSO-d₆) δ 8.17 (s, 1H), 8.03-8.01 (m, 1H), 7.78-7.76 (m, 1H), 7.69 (s, 2H), 6.78 (d, J = 27.2 Hz, 1H), 6.07 (d, J = 20.0 Hz, 1H), 5.62 (d, J = 10.1 Hz, 1H), 4.91 (s, 1H), 4.18-3.91 (m, 5H), 3.73 (s, 3H), 3.60-3.49 (m, 5H), 3.14- 3.09 (m, 2H), 2.87 (m, 1H), 2.50-2.32 (m, 1H), 1.64-1.56 (m, 4H). | 536.35 |
| 1-((3S,4R)-4-(3-((4- amino-5-(5-chloro-6- methoxypyridin-2-yl)- 7-methyl-7H- pyrrolo[2,3- d]pyrimidin-6- yl)ethynyl)azetidin-1- yl)-3-fluoropiperidin- 1-yl)prop-2-en-1-one | 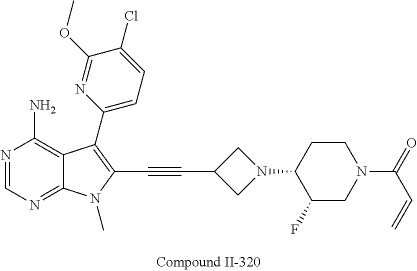 [SMILES: C=CC(=O)N1CC[C@@H](N2CC(C#Cc3c(-c4ccc(Cl)c(OC)n4)c4c(N)ncnc4n3C)C2)[C@@H](F)C1] | ¹H NMR (400 MHz, DMSO-d₆) δ 8.17 (s, 1H), 8.02-8.00 (m, 1H), 7.78-7.68 (m, 1H), 7.67 (s, 2H), 6.86 (d, J = 24 Hz, 1H), 6.07 (d, J = 16 Hz, 1H), 5.69 (d, J = 12 Hz, 1H), 4.76-4.64 (m, 1H), 4.41-4.20 (m, 2H), 4.00- 3.94 (m, 4H), 3.73-3.58 (m, 6H), 3.40-3.22 (m, 3H), 3.08-2.81 (m, 1H), 1.45-1.31 (m, 2H). | 524.30 |
| 1-((3S,4R)-4-(3-((4- amino-5-(5- chloropyridin-2-yl)-7- methyl-7H- pyrrolo[2,3- d]pyrimidin-6- yl)ethynyl)azetidin-1- yl)-3- hydroxypiperidin-1- yl)prop-2-en-1-one | 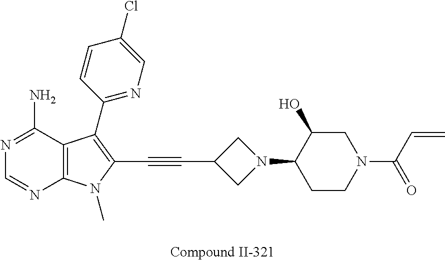 [SMILES: C=CC(=O)N1CC[C@@H](N2CC(C#Cc3c(-c4ccc(Cl)cn4)c4c(N)ncnc4n3C)C2)[C@@H](O)C1] | ¹H NMR (400 MHz, DMSO-d₆) δ 8.76 (d, J = 2.5 Hz, 1H), 8.40 (d, J = 8.8 Hz, 1H), 8.16 (s, 1H), 8.09 (dd, J = 8.7, 2.8 Hz, 1H), 7.39 (s, 1H), 6.85-6.68 (m, 1H), 6.05 (dd, J = 16.8, 2.4 Hz, 1H), 5.67-5.59 (m, 1H), 4.58- 4.45 (m, 1H), 4.04-3.78 (m, 1H), 3.76-3.54 (m, 8H), 3.30- 3.16 (m, 3H), 2.97-2.89 (m, 1H), 2.44-2.36 (m, 1H), 1.56- 1.42 (m, 2H). | 492.3 |
| 5-(6-methoxy-5- phenoxypyridin-2-yl)- 7-methyl-6-((1-(1- (vinylsulfonyl) piperidin-4-yl) azetidin-3-yl)ethynyl)- 7H-pyrrolo[2,3- d]pyrimidin-4-amine | 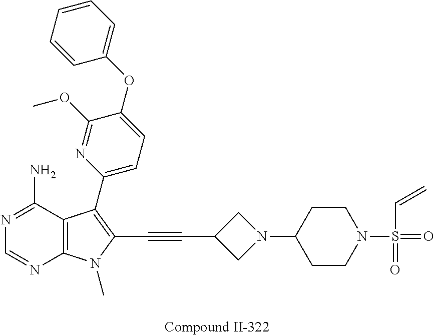 [SMILES: C=CS(=O)(=O)N1CCC(N2CC(C#Cc3c(-c4ccc(Oc5ccccc5)c(OC)n4)c4c(N)ncnc4n3C)C2)CC1] | ¹H NMR (400 MHz, Chloroform-d) δ 8.35 (s, 1H), 7.77 (d, J = 8.2 Hz, 1H), 7.44- 7.35 (m, 2H), 7.27 (s, 1H), 7.22- 7.13 (m, 1H), 7.10-7.03 (m, 2H), 6.44 (dd, J = 16.6, 9.9 Hz, 1H), 6.25 (d, J = 16.6 Hz, 1H), 6.03 (d, J = 9.9 Hz, 1H), 4.07 (s, 3H), 3.85 (s, 3H), 3.74-3.65 (m, 2H), 3.51 (tt, J = 10.7, 6.2 Hz, 3H), 3.16-3.08 (m, 2H), 2.90 (ddd, J = 12.3, 9.1, 3.3 Hz, 2H), 2.22 (dt, J = 8.3, 4.4 Hz, 1H), 1.95-1.62 (m, 2H), 1.43 (dtd, J = 12.8, 8.8, 3.7 Hz, 2H). | 600.3 |
| (3S,4R)-4-(3-((4- amino-5-(6-methoxy- 5-phenoxypyridin-2- yl)-7-methyl-7H- pyrrolo[2,3- d]pyrimidin-6- yl)ethynyl)azetidin-1- yl)-1- (vinylsulfonyl) piperidin-3-ol | 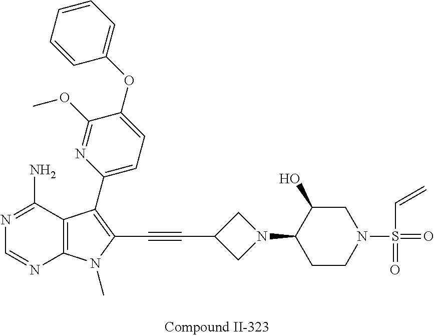 [SMILES: C=CS(=O)(=O)N1CC[C@@H](N2CC(C#Cc3c(-c4ccc(Oc5ccccc5)c(OC)n4)c4c(N)ncnc4n3C)C2)[C@@H](O)C1] | ¹H NMR (400 MHz, DMSO-d₆) δ 8.17 (s, 1H), 8.02 (br, 1H), 7.81 (d, J = 8.2 Hz, 1H), 7.55 (d, J = 8.2 Hz, 1H), 7.39 (t, J = 7.8 Hz, 2H), 7.13 (t, J = 7.4 Hz, 1H), 7.02 (d, J = 8.0 Hz, 2H), 6.80 (dd, J = 16.5, 10.0 Hz, 1H), 6.13- 6.04 (m, 2H), 4.77 (d, J = 3.7 Hz, 1H), 3.93 (s, 3H), 3.74 (s, 3H), 3.70-3.62 (m, 2H), 3.60 (t, J = 6.5 Hz, 1H), 3.53 (q, J = 7.1 Hz, 1H), 3.16 (dt, J = 19.9, 7.0 Hz, 3H), 3.02 (ddt, J = 25.8, 11.6, 4.8 Hz, 2H), 2.88 (ddd, J = 11.6, 7.1, 3.9 Hz, 1H), 2.32 (dt, J = 6.9, 3.1 Hz, 1H), 1.64-1.52 (m, 1H), 1.44 (tt, J = 9.7, 3.7 Hz, 1H). | 616.3 |
| 1-(4-(3-((4-amino-5- (5-chloro-6- methoxypyridin-2-yl)- 7-methyl-7H- pyrrolo[2,3- d]pyrimidin-6- yl)ethynyl)azetidin-1- yl)piperidin-1-yl)prop- 2-en-1-one | 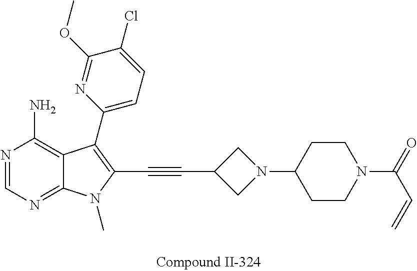 [SMILES: C=CC(=O)N1CCC(N2CC(C#Cc3c(-c4ccc(Cl)c(OC)n4)c4c(N)ncnc4n3C)C2)CC1] | ¹H NMR (400 MHz, DMSO-d6) δ 8.18 (s, 1H), 8.02 (d, J = 8.1 Hz, 1H), 7.76 (d, J = 8.1 Hz, 1H), 7.64 (s, 2H), 6.79 (dd, J = 16.7, 10.5 Hz, 1H), 6.07 (dd, J = 16.7, 2.5 Hz, 1H), 5.65 (dd, J = 10.5, 2.5 Hz, 1H), 4.01 (s, 4H), 3.84 (d, J = 12.5 Hz, 1H), 3.74 (s, 3H), 3.61 (t, J = 6.7 Hz, 2H), 3.54 (p, J = 7.3, 6.8 Hz, 1H), 3.20 (t, J = 11.8 Hz, 1H), 3.12 (t, J = 6.2 Hz, 2H), 3.02 (t, J = 11.3 Hz, 1H), 2.32 (dd, J = 9.2, 5.1 Hz, 1H), 1.64 (s, 1H), 1.10 (s, 2H). | 506.4 |
| 1-(4-(3-((4-amino-5- (5-chloropyridin-2-yl)- 7-methyl-7H- pyrrolo[2,3- d]pyrimidin-6- yl)ethynyl)azetidin-1- yl)piperidin-1-yl)prop- 2-en-1-one | 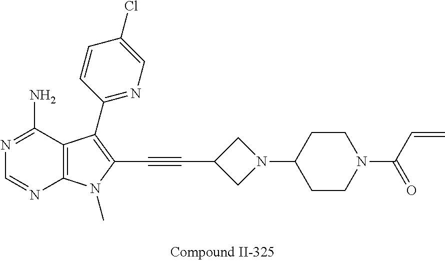 [SMILES: C=CC(=O)N1CCC(N2CC(C#Cc3c(-c4ccc(Cl)cn4)c4c(N)ncnc4n3C)C2)CC1] | ¹H NMR (400 MHz, Chloroform- d) δ 8.76 (s, 1H), 8.42-8.35 (m, 1H), 8.16 (s, 1H), 8.08 (d, J = 9.0 Hz, 1H), 7.41 (s, 1H), 6.86-6.75 (m, 1H), 6.08 (d, J = 16.6 Hz, 1H), 5.69-5.61 (m, 1H), 4.01 (s, 1H), 3.85 (s, 1H), 3.74 (s, 3H), 3.62 (s, 3H), 3.17 (s, 3H), 3.02 (s, 1H), 2.33 (s, 1H), 1.66 (s, 2H), 1.11 (s, 2H). | 476.25 |
| 1-(4-(3-((4-amino-5- (5-chloro-6- methylpyridin-2-yl)-7- isopropyl-7H- pyrrolo[2,3- d]pyrimidin-6- yl)ethynyl)azetidin-1- yl)piperidin-1-yl)prop- 2-en-1-one | 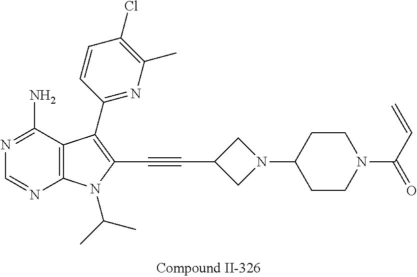 [SMILES: C=CC(=O)N1CCC(N2CC(C#Cc3c(-c4ccc(Cl)c(C)n4)c4c(N)ncnc4n3C(C)C)C2)CC1] | ¹H NMR (400 MHz, Chloroform-d) δ 8.29 (s, 1H), 8.14 (d, J = 8.5 Hz, 1H), 7.69 (d, J = 8.5 Hz, 1H), 6.60 (dd, J = 16.8, 10.6 Hz, 1H), 6.28 (dd, J = 16.9, 2.0 Hz, 1H), 5.70 (dd, J = 10.6, 2.0 Hz, 1H), 5.26 (hept, J = 7.1 Hz, 1H), 4.25 (d, J = 13.2 Hz, 1H), 3.88 (d, J = 12.7 Hz, 1H), 3.73 (t, J = 7.0 Hz, 2H), 3.56 (p, J = 7.5 Hz, 1H), 3.20 (h, J = 11.0 Hz, 4H), 2.69 (s, 3H), 2.52 (s, 1H), 2.36 (tt, J = 8.4, 3.8 Hz, 1H), 1.71 (d, J = 7.0 Hz, 6H), 1.33 (dtd, J = 13.2, 9.3, 3.9 Hz, 2H). | 518.25 |
| 1-(4-(3-((4-amino-5- (5-chloro-6- methoxypyridin-2-yl)- 7-isopropyl-7H- pyrrolo[2,3- d]pyrimidin-6- yl)ethynyl)azetidin-1- yl)piperidin-1-yl)prop- 2-en-1-one | 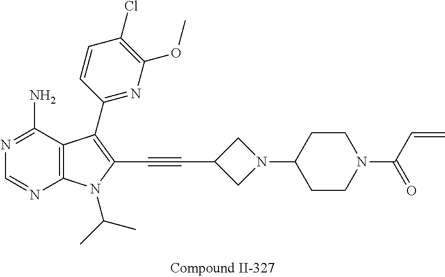 [SMILES: C=CC(=O)N1CCC(N2CC(C#Cc3c(-c4ccc(Cl)c(OC)n4)c4c(N)ncnc4n3C(C)C)C2)CC1] | ¹H NMR (400 MHz, Methanol- d₄) δ 8.17 (s, 1H), 7.90 (d, J = 8.1 Hz, 1H), 7.77 (d, J = 8.1 Hz, 1H), 6.79 (dd, J = 16.7, 10.6 Hz, 1H), 6.19 (d, J = 16.8 Hz, 1H), 5.75 (d, J = 10.6 Hz, 1H), 5.24 (p, J = 7.0 Hz, 1H), 4.38 (d, J = 13.6 Hz, 1H), 4.09 (d, J = 1.4 Hz, 4H), 3.74 (t, J = 7.4 Hz, 2H), 3.62 (p, J = 7.4 Hz, 1H), 3.31 (d, J = 7.4 Hz, 3H), 3.02-2.91 (m, 1H), 2.46 (s, 1H), 1.84 (d, J = 13.1 Hz, 2H), 1.72 (d, J = 6.9 Hz, 6H), 1.22 (t, J = 10.7 Hz, 2H). | 534.3 |
| 1-((3R,4S)-4-(3-((4- amino-5-(5-chloro-6- methylpyridin-2-yl)-7- isopropyl-7H- pyrrolo[2,3- d]pyrimidin-6- yl)ethynyl)azetidin-1- yl)-3-fluoropiperidin- 1-yl)prop-2-en-1-one | 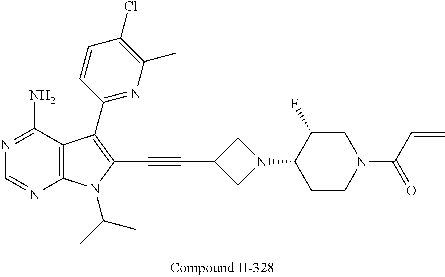 [SMILES: C=CC(=O)N1CC[C@H](N2CC(C#Cc3c(-c4ccc(Cl)c(C)n4)c4c(N)ncnc4n3C(C)C)C2)[C@H](F)C1] | ¹H NMR (400 MHz, Chloroform- d) δ 8.29 (s, 1H), 8.15 (d, J = 8.5 Hz, 1H), 7.70 (d, J = 8.5 Hz, 1H), 6.59 (dd, J = 16.8, 10.6 Hz, 1H), 6.30 (d, J = 18.7 Hz, 1H), 5.74 (dd, J = 10.6, 1.9 Hz, 1H), 5.26 (p, J = 6.9 Hz, 1H), 4.64 (d, J = 47.7 Hz, 1H), 4.38-4.12 (m, 1H), 3.82 (dt, J = 23.2, 7.1 Hz, 2H), 3.64 (p, J = 7.6 Hz, 1H), 3.56-3.37 (m, 1H), 3.30 (q, J = 8.0, 7.0 Hz, 2H), 3.21 (s, 1H), 2.69 (s, 3H), 2.56 (d, J = 28.5 Hz, 1H), 1.72- 1.66 (m, 9H). | 536.25 |
| 1-((2S)-4-(3-((4- amino-5-(6-methoxy- 5-phenoxypyridin-2- yl)-7-methyl-7H- pyrrolo[2,3- d]pyrimidin-6- yl)ethynyl)azetidin-1- yl)-2- (hydroxymethyl) piperidin-1-yl)prop-2- en-1-one | 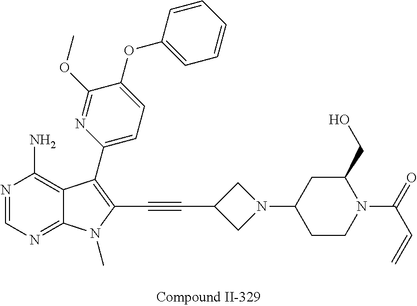 [SMILES: C=CC(=O)N1CCC(N2CC(C#Cc3c(-c4ccc(Oc5ccccc5)c(OC)n4)c4c(N)ncnc4n3C)C2)C[C@H]1CO] | ¹H NMR (400 MHz, DMSO-d₆) δ 8.17 (s, 1H), 7.82 (d, J = 8.2 Hz, 1H), 7.54 (d, J = 8.2 Hz, 1H), 7.39 (t, J = 7.8 Hz, 2H), 7.13 (t, J = 7.4 Hz, 1H), 7.02 (d, J = 8.0 Hz, 2H), 6.77 (dd, J = 16.9, 10.6 Hz, 1H), 6.04 (d, J = 16.3 Hz, 1H), 5.60 (d, J = 10.6 Hz, 1H), 4.90 (s, 1H), 4.20-3.90 (s, 6H), 3.74 (s, 3H), 3.61-3.40 (m, 4H), 3.15-2.77 (m, 3H), 2.42 (s, 1H), 1.65-1.18 (m, 4H). | 594.3 |
| 1-((3S,4R)-4-(3-((4- amino-5-(6-methoxy- 5-phenoxypyridin-2- yl)-7-methyl-7H- pyrrolo[2,3- d]pyrimidin-6- yl)ethynyl)azetidin-1- yl)-3-fluoropiperidin- 1-yl)prop-2-en-1-one | 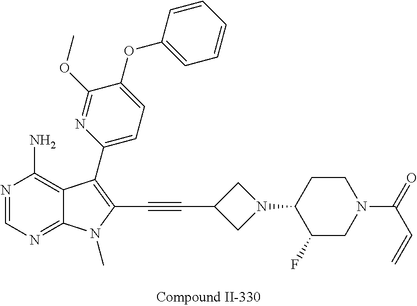 [SMILES: C=CC(=O)N1CC[C@@H](N2CC(C#Cc3c(-c4ccc(Oc5ccccc5)c(OC)n4)c4c(N)ncnc4n3C)C2)[C@@H](F)C1] | ¹H NMR (400 MHz, Chloroform- d) δ 8.35 (s, 1H), 7.78 (d, J = 8.2 Hz, 1H), 7.40 (t, J = 7.7 Hz, 2H), 7.30 (s, 1H), 7.18 (t, J = 7.4 Hz, 1H), 7.07 (d, J = 8.0 Hz, 2H), 6.57 (dd, J = 16.8, 10.6 Hz, 1H), 6.29 (d, J = 16.9 Hz, 1H), 5.77-5.69 (m, 1H), 4.60 (d, J = 47.9 Hz, 1H), 4.25 (d, J = 49.2 Hz, 1H), 4.07 (s, 3H), 3.87-3.75 (m, 5H), 3.60 (p, J = 7.7 Hz, 1H), 3.30-3.11 (m, 3H), 2.56 (s, 1H), 2.30-1.70 (d, J = 93.8 Hz, 4H). | 582.3 |

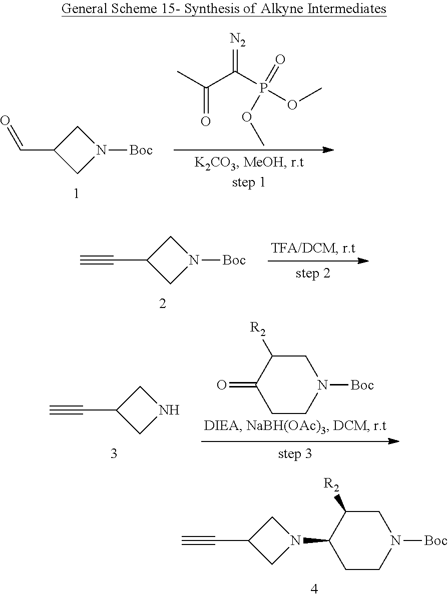

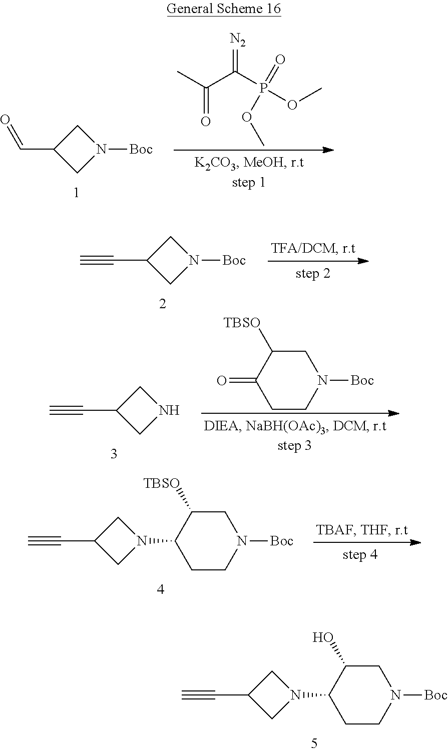

### Example 27

### (3R,4S)-tert-butyl 4-(3-ethynylazetidin-1-yl)-3-hydroxypiperidine-1-carboxylate

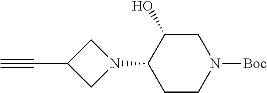 [SMILES: C#CC1CN([C@H]2CCN(C(=O)OC(C)(C)C)C[C@H]2O)C1]

### Step 1

A round bottomed flask was charged with tert-butyl 3-formylazetidine-1-carboxylate (30 g, 161 mmol), K₂CO₃ (66.6 g, 483 mmol), MeOH (300 mL) and a stir bar. The mixture was cooled to 0° C. and dimethyl-(1-diazo-2-oxopropyl)-phosphonate (46.2 g, 241 mmol) was added, and the solution was stirred for 4 h at room temperature. The reaction mixture was filtered and washed with DCM. The organic layers were concentrated in vacuo. The resulting crude material was purified by silica gel chromatography (100 g column; eluting with EA/PE=1/20). Concentration in vacuo resulted in tert-butyl 3-ethynylazetidine-1-carboxylate (28.0 g, 154 mmol).

### Step 2

A round bottomed flask was charged with tert-butyl 3-ethynylazetidine-1-carboxylate (2 g, 11.0 mmol), DCM (12 mL), TFA (3 mL) and a stir bar. The solution was stirred for 1 h at room temperature. The reaction mixture was concentrated in vacuo resulted in 3-ethynylazetidine as a yellow oil, (Theoretical Mass:890 mg) The product was used in the next step directly without further purification.

### Step 3

A round bottomed flask was charged with 3-ethynylazetidine (890 mg, 10.9 mmol), DCM (10 mL) and a stir bar. The pH of solution was adjusted to 6-8 with DIEA. Then the tert-butyl 3-[(tert-butyldimethylsilyl)-oxy]-4-oxopiperidine-1-carboxylate (5.37 g, 16.3 mmol) was added and the mixture was cooled to 0° C. Then NaBH(OAc)₃ (2.75 g, 13.0 mmol) was added. The solution was stirred for 2 h at room temperature. The reaction mixture was diluted with H₂O (100 mL), and the aqueous phase was extracted with EA (150 mL) three times. The combined organic layers were washed with brines, dried over sodium sulfate, filtered, and concentrated in vacuo. The resulting crude material was purified by silica gel chromatography (20 g column; eluting with EA/PE=1/8). Concentration in vacuo resulted in tert-butyl 3-[(tert-butyldimethylsilyl)oxy]-4-(3-ethynylazetidin-1-yl)piperidine-1-carboxylate (3.30 g, 8.36 mmol).

### Step 4

A round bottomed flask was charged with tert-butyl 3-[(tert-butyldimethylsilyl)-oxy]-4-(3-ethynylazetidin-1-yl)piperidine-1-carboxylate (1.5 g, 3.80 mmol), THE (4 mL) TBAF (1.98 g, 7.60 mmol, 1 mol/L in THF), and a stir bar. The solution was stirred for 2 h at room temperature. The resulting crude material was purified by silica gel chromatography (5 g column; eluting with EA/PE=2/3). Concentration in vacuo resulted in tert-butyl 4-(3-ethynylazetidin-1-yl)-3-hydroxypiperidine-1-carboxylate (980 mg, 3.49 mmol).

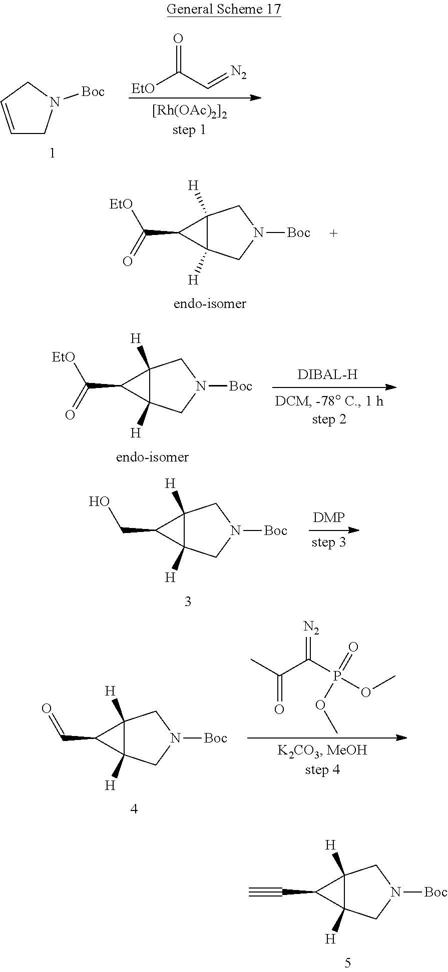

### Example 28

### (1R,5S,6s)-tert-butyl 6-ethynyl-3-aza-bicyclo[3.1.0]hexane-3-carboxylate

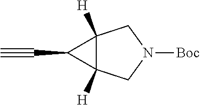

### Step 1

A round bottomed flask was charged with tert-butyl 2,5-dihydro-1H-pyrrole-1-carboxylate (15 g, 88.6 mmol) and, bis((acetyloxy)rhodio acetate) (1.95 g, 4.43 mmol) and a stirbar. 300 mL of DCM was added and stirred under N₂, then ethyl 2-diazoacetate (13.1 g, 115 mmol) dissolved in 100 mL of DCM was slowly added dropwise for 3 h at r.t. After stirring 24 hours, the mixture was filtered through Celite and concentrated under reduced pressure, the residue was purified by silica gel column chromatography, eluted with PE/EtOAc (6:1-3:1) to afford 8 g of exo-isomer and 4 g of endo-isomer.

### Step 2

To a stirred solution of 3-tert-butyl6-methyl 3-azabicyclo[3.1.0]hexane-3,6-dicarboxylate (10.3 g, 42.69 mmol, 1 equiv) in DCM were added DIBAL-H (15.2 g, 106.72 mmol, 2.5 equiv) dropwise at −78° C. under nitrogen atmosphere. The reaction was monitored by TLC. The reaction was quenched with Water at −78° C. The precipitated solids were collected by filtration and washed with DCM. The resulting mixture was concentrated under reduced pressure. The residue was purified by silica gel column chromatography, eluted with PE/EtOAc (4:1) to afford tert-butyl 6-(hydroxymethyl)-3-azabicyclo[3.1.0]hexane-3-carboxylate (7.5 g, 82.4%).

### Step 3

A solution of tert-butyl 6-(hydroxymethyl)-3-azabicyclo[3.1.0]hexane-3-carboxylate (7.5 g, 35.17 mmol, 1 equiv) and Dess-Martin reagent (29.8 g, 70.33 mmol, 2 equiv) in DCM was stirred for 3 h at room temperature. The reaction was monitored by TLC. The reaction mixture was diluted with NaHCO₃ (300 mL), and the aqueous phase was extracted with DCM (500 mL) three times. The combined organic layers were washed with brine, dried over sodium sulfate, filtered, and concentrated in vacuo. The residue was purified by silica gel column chromatography, eluted with PE/EtOAc (5:1) to afford tert-butyl 6-formyl-3-azabicyclo[3.1.0]hexane-3-carboxylate (6.5 g, 87.5%).

### Step 4

To a stirred solution of tert-butyl 6-formyl-3-azabicyclo[3.1.0]hexane-3-carboxylate (6.5 g, 30.77 mmol, 1 equiv) and K₂CO₃ (12.8 g, 92.30 mmol, 3 equiv) in MeOH was added dimethyl (1-diazo-2-oxopropyl)phosphonate (8.9 g, 46.15 mmol, 1.5 equiv) dropwise at 0° C. The resulting mixture was stirred for 2 h at room temperature. The reaction was monitored by TLC. The reaction mixture was diluted with H₂O (300 mL), and the aqueous phase was extracted with DCM (500 mL) three times. The combined organic layers were washed with brine, dried over sodium sulfate, filtered, and concentrated in vacuo. The residue was purified by silica gel column chromatography, eluted with PE/EtOAc (15:1) to afford tert-butyl 6-ethynyl-3-azabicyclo[3.1.0]hexane-3-carboxylate (4 g, 63%).

### Example 29

### Selected Additional Compounds

Additional compounds prepared by substantially the same methods as those described herein are listed in Table 18, below.

TABLE 18
Selected Compounds

| Compound | Structure |
|---|---|
| III-1 | 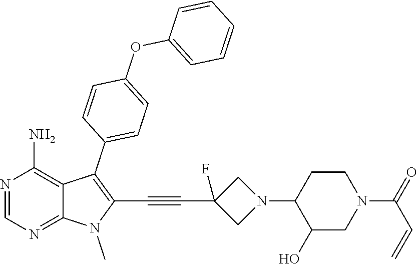 [SMILES: C=CC(=O)N1CCC(N2CC(F)(C#Cc3c(-c4ccc(Oc5ccccc5)cc4)c4c(N)ncnc4n3C)C2)C(O)C1] |
| III-2 | 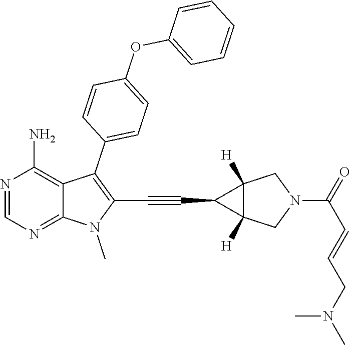 [SMILES: CN(C)C/C=C/C(=O)N1C[C@@H]2[C@@H](C#Cc3c(-c4ccc(Oc5ccccc5)cc4)c4c(N)ncnc4n3C)[C@@H]2C1] |
| III-3 | 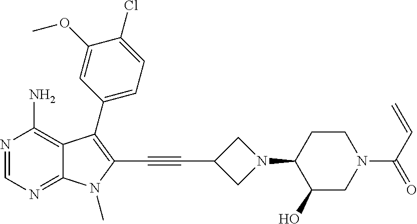 [SMILES: C=CC(=O)N1CC[C@H](N2CC(C#Cc3c(-c4ccc(Cl)c(OC)c4)c4c(N)ncnc4n3C)C2)[C@H](O)C1] |
| III-4 | 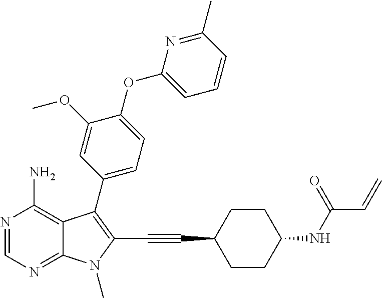 [SMILES: C=CC(=O)N[C@H]1CC[C@H](C#Cc2c(-c3ccc(Oc4cccc(C)n4)c(OC)c3)c3c(N)ncnc3n2C)CC1] |
| III-5 | 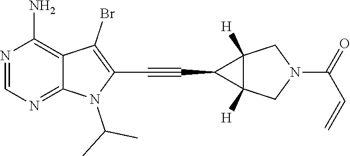 [SMILES: C=CC(=O)N1C[C@@H]2[C@@H](C#Cc3c(Br)c4c(N)ncnc4n3C(C)C)[C@@H]2C1] |
| III-6 | 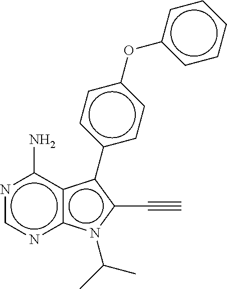 |
| III-7 | 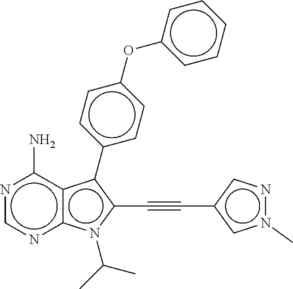 |
| III-8 | 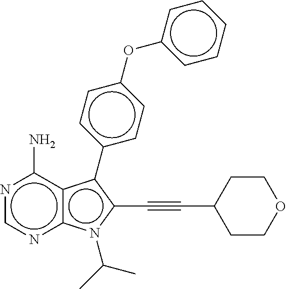 |
| III-9 | 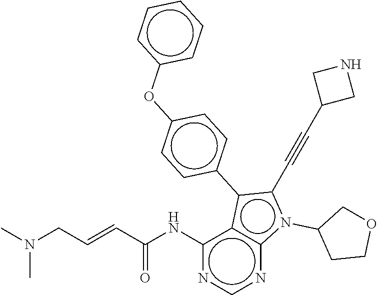 |
| III-18 | 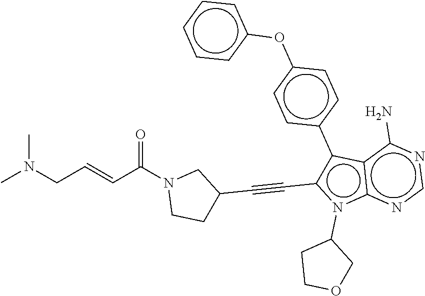 |
| III-23 | 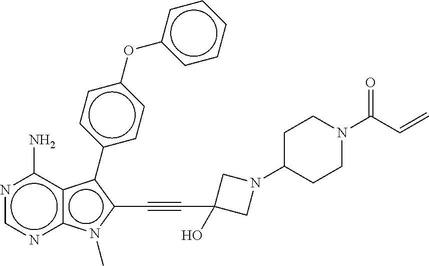 |
| III-25 | 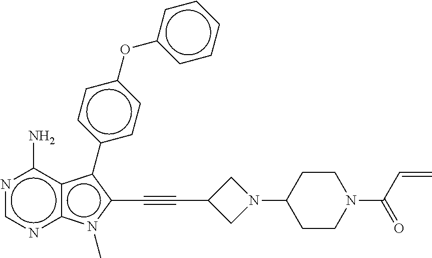 |
| III-27 | 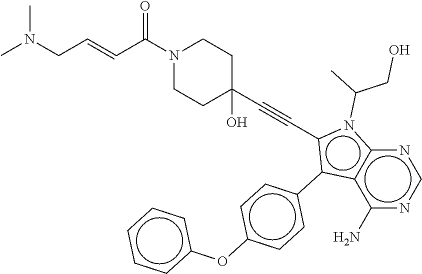 |
| III-30 | 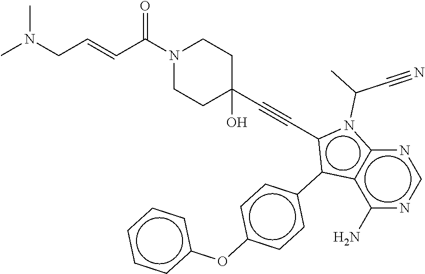 |
| III-31 | 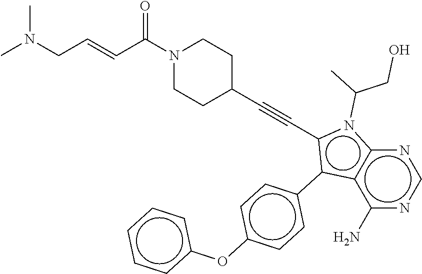 |
| III-32 | 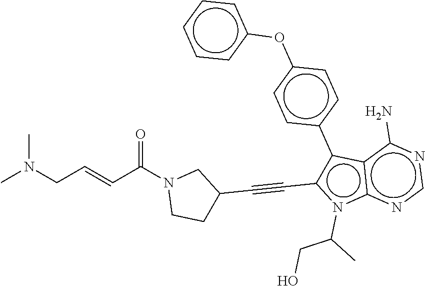 |
| III-33 | 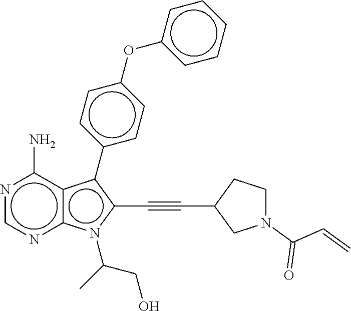 |
| III-34 | 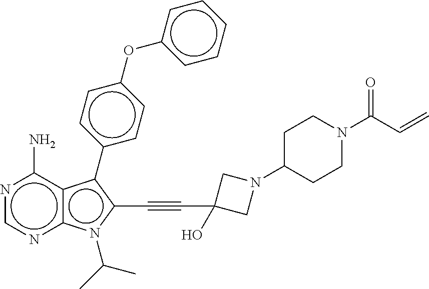 |
| III-38 | 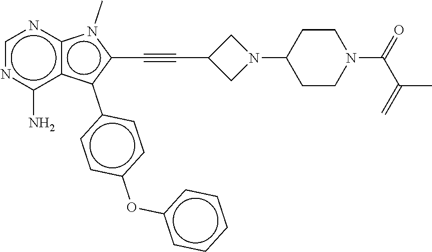 |
| III-42 | 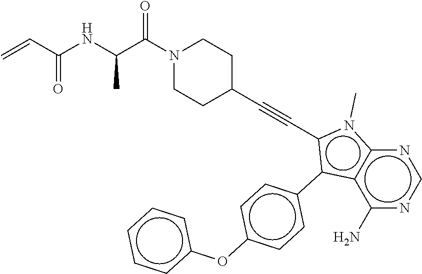 |
| III-43 | 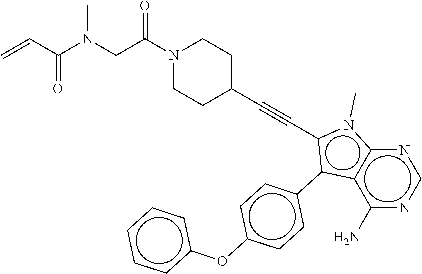 |
| III-44 | 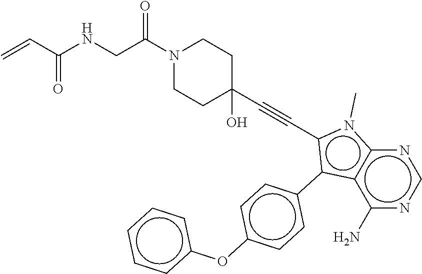 |
| III-45 | 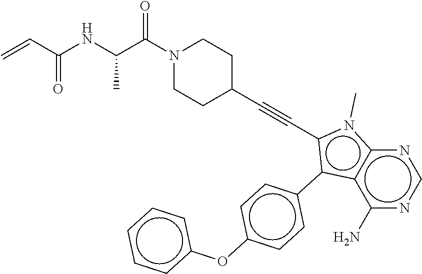 |
| III-46 | 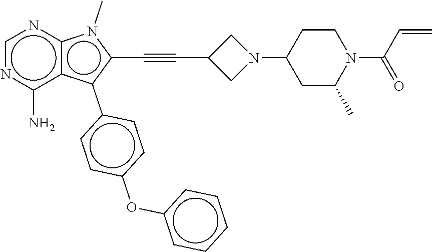 |
| III-47 | 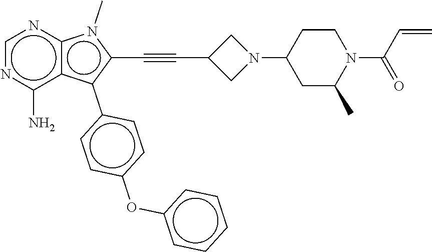 |
| III-49 | 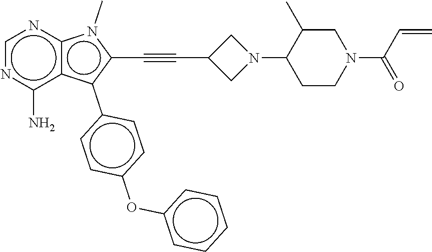 |
| III-51 | 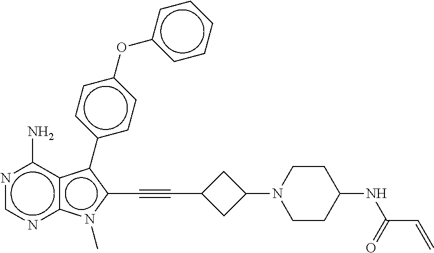 |
| III-53 | 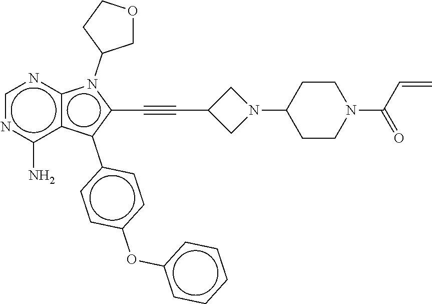 |
| III-54 | 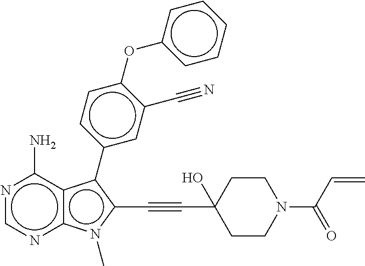 |
| III-55 | 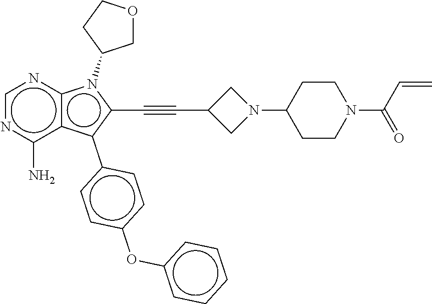 |
| III-56 | 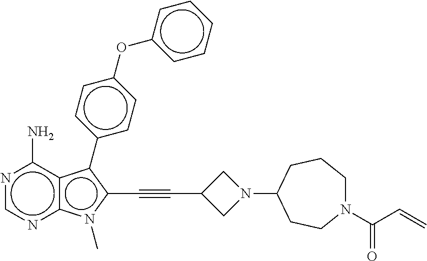 |
| III-59 | 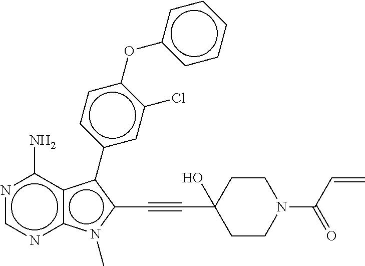 |
| III-60 | 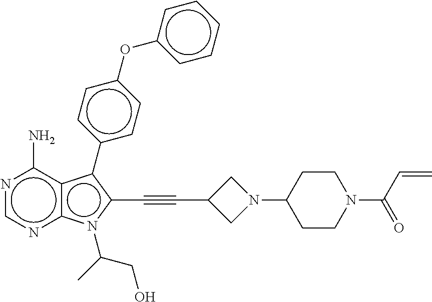 |
| III-62 | 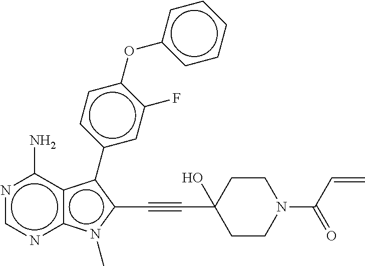 |
| III-63 | 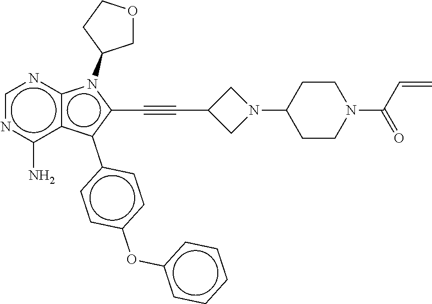 |
| III-64 | 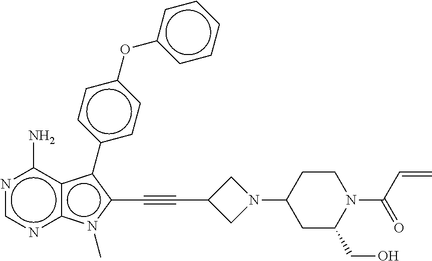 |
| III-65 | 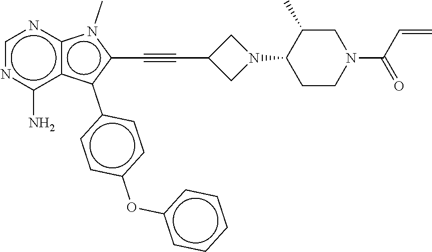 |
| III-66 | 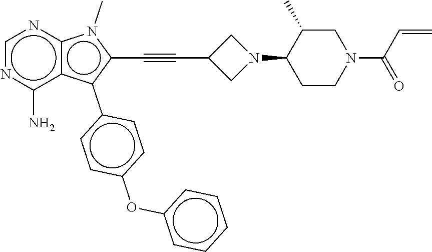 |
| III-67 | 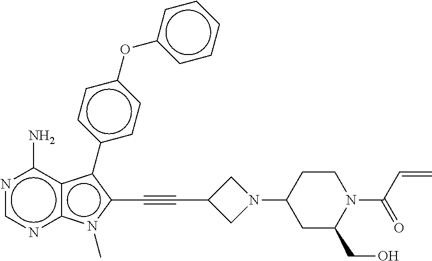 |
| III-68 | 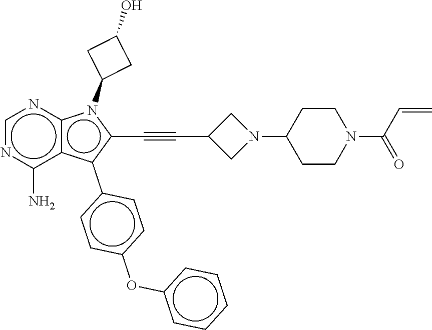 |
| III-69 | 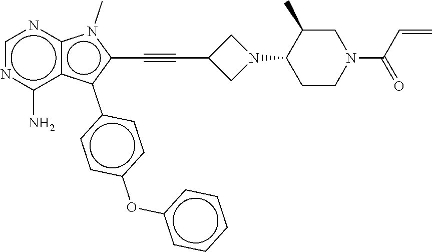 |
| III-70 | 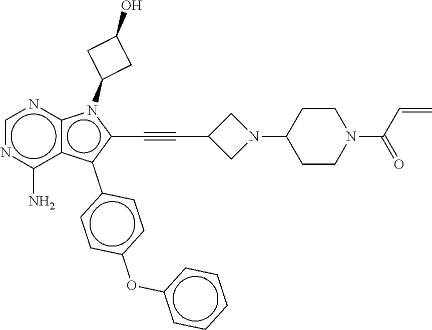 |
| III-73 | 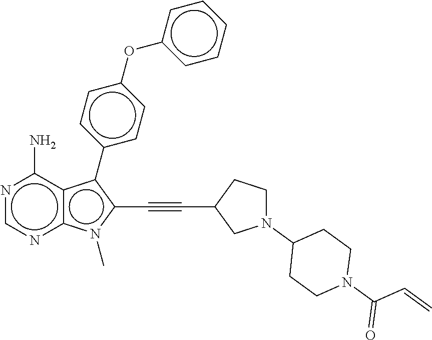 |
| III-74 | 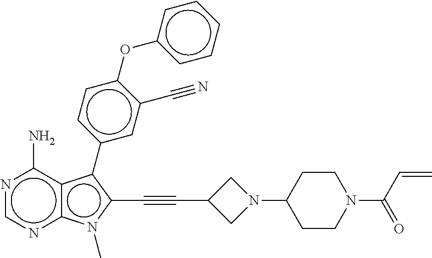 |
| III-75 | 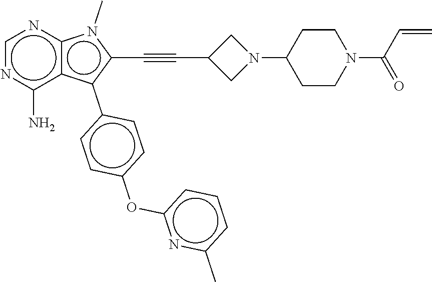 |
| III-76 | 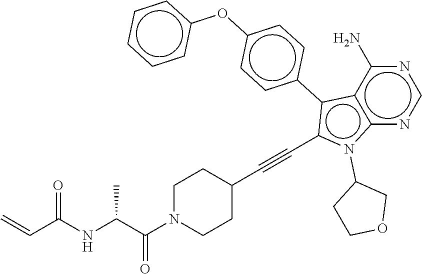 |
| III-80 | 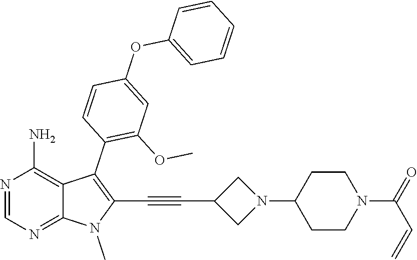 [SMILES: C=CC(=O)N1CCC(N2CC(C#Cc3c(-c4ccc(Oc5ccccc5)cc4OC)c4c(N)ncnc4n3C)C2)CC1] |
| III-82 | 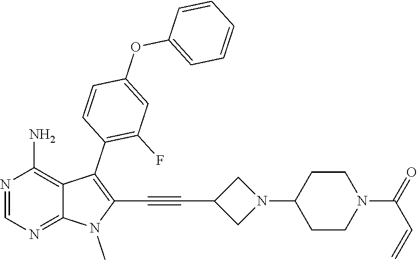 [SMILES: C=CC(=O)N1CCC(N2CC(C#Cc3c(-c4ccc(Oc5ccccc5)cc4F)c4c(N)ncnc4n3C)C2)CC1] |
| III-83 | 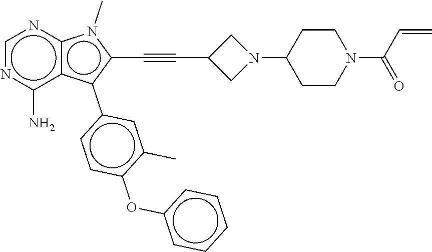 |
| III-85 | 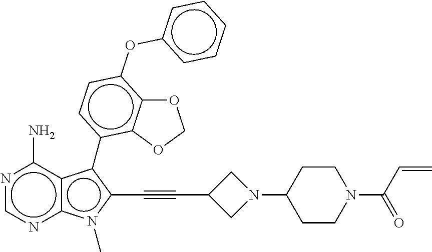 |
| III-86 | 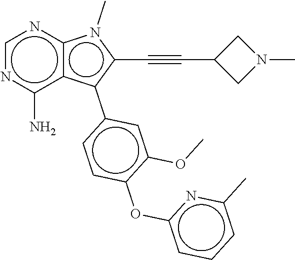 |
| III-87 | 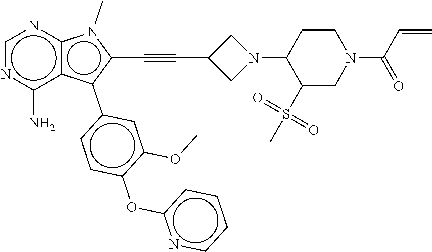 |
| III-88 | 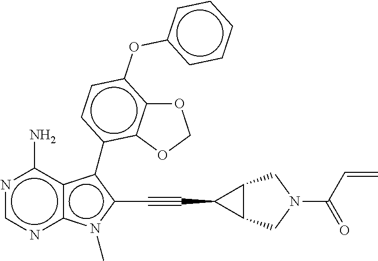 |
| III-89 | 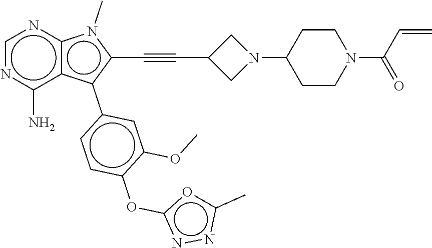 |
| III-90 | 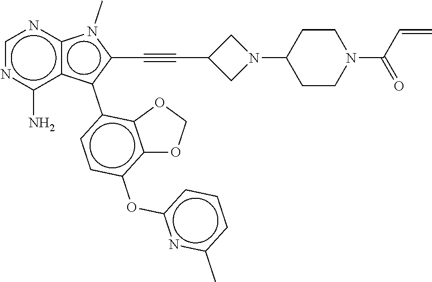 |
| III-91 | 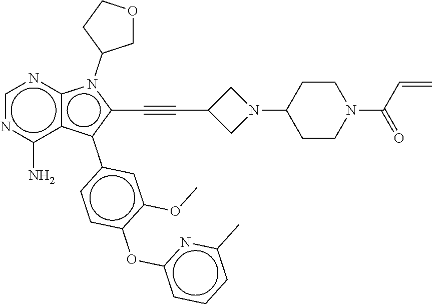 |
| III-92 | 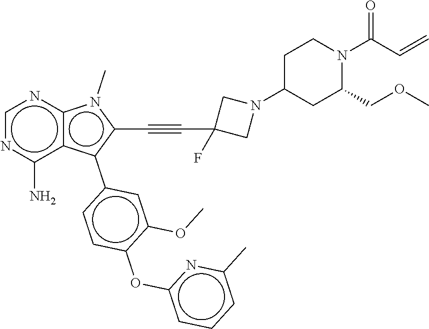 |
| III-93 | 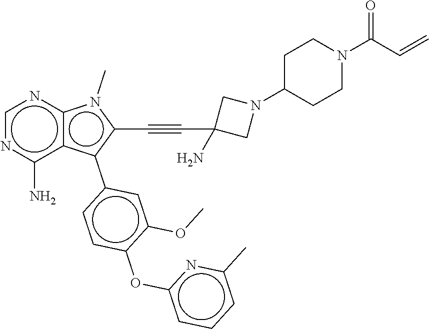 |
| III-94 | 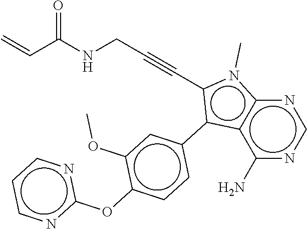 |
| III-95 | 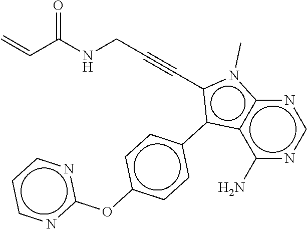 |

### Example 30

### SRC Biochemical Assay Protocol

Src biochemical kinase activity was measured using a surrogate fluorescent peptide substrate [FLPeptide4; 5-FAM-EGIYGVLFKKK-CONH₂ (PerkinElmer Cat. No. 760348)] with a microfluidic mobility shift assay. Reactions were performed in 384-well flat bottom polystyrene microplates (Corning Cat. No. 3570) at a final volume of 20 μL in 50 mM HEPES pH 7.4, 1 mM EGTA, 10 mM MgCl₂, 0.01% Brij-35, 2 mM DTT, 0.05% BSA, 1% DMSO. Inhibition experiments were performed with compounds of the invention varying from 0.0017-100 μM with 0.26 nM SRC, 1.5 μM FLPeptide4, and 50 μM ATP. Specifically, 15 μl 1.3× mixtures of SRC+FLPeptide4+compounds were preincubated for 30 minutes at room temperature followed by addition of 5 μL 4×ATP. Reactions were carried out for 90 minutes then quenched with 40 μl 500 mM EDTA. Phosphorylated peptide product was measured with a mobility shift assay using a LabChip® EZ Reader system (PerkinElmer Cat. No. 122919). Inhibition ratios were calculated relative to neutral controls and IC₅₀ values were obtained from dose response data fitted to a 4-parameter logistic model.

### Example 31

### Cell Viability Protocol

RBE or SNU-1079 cells were cultured in T-75 or T-175 flasks at 37° C., 5% CO₂, 95% relative humidity in RPMI 1640 medium with 10% FBS and 1% Penicillin/Streptomycin (media) and detached with 0.25% trypsin/EDTA at 80-90% confluency. Cells were then seeded in 40 μL media per well in 384-well flat-bottom polystyrene microplates (Corning Cat. No. 3570) at a seeding density of 1200 cells/well and incubated for 24 hours at 37° C. with 5% CO₂, 95% relative humidity. On the next day, test compounds were prepared in 10 mM DMSO stock solution and diluted to the indicated concentrations in a 384-well compound source plate. DMSO was employed as a vehicle control. Compounds (40 nL) were transferred to 384-well cell culture plate(s) using the liquid handler Echo550. Plates were incubated at 37° C. with 5% CO₂ for 72 hour or 120 hour (as indicated). CellTiterGlo reagent was warmed to 37° C. and added into each well (40 μL per well). Plates were incubated at room temperature for 30 min followed by reading on the EnSpire or EnVision plate reader. Relative viability (% remaining activity) was calculated relative to DMSO control and IC₅₀ values were obtained from dose-response data fitted to a 4-parameter curve.

### Example 32

### Cellular Proliferation Protocol

Cellular phospho-S6RP (Ser 235/236) was measured using a Fluorescence Resonance Energy Transfer (FRET) sandwich assay format (Cisbio HTRF Cat. No. 64RP6PEG). This format uses 2 different, labelled antibodies (Eu³⁺-Cryptate donor and d2 acceptor) that positively modulate a specific signal in proportion to phospho-S6RP (Ser 235/236) directly in cells. RBE or SNU-1079 cells were cultivated in T-75 flasks at 37° C., 5% CO₂, 95% relative humidity in RPMI 1640 medium with 10% FBS and 1% Penicillin/Streptomycin and detached with trypsin/EDTA at 80-90% confluency. Cells were then seeded in 40 μL volume per well in 384-well flat-bottom polystyrene microplates (Corning Cat. No. 3570) at a seeding density of 2000 cells/well for RBE and 3000 cells/well for SNU-1079 followed by 24 hour incubation at 37° C., 5% CO₂, 95% relative humidity. On the next day, compounds were diluted to 2× the indicated concentrations and 40 nL of these treatment compounds, along with DMSO vehicle control, were added to the cells (final concentration of compounds is then the indicated concentrations). Treatment was carried out for 6 hours at 37° C., 5% CO₂, 95% relative humidity after which the plates were cooled to room temperature before aspirating the medium. Cells were then lysed on a shaker for 45 minutes at room temperature with 20 μL of 1× supplemented lysis buffer per well. After lysis, 5 μL of pre-mixed antibodies was added per well. Plates were covered with black sealing film and incubated overnight at room temperature on a shaker before being read on a PerkinElmer EnVision microplate reader. Percent activation was calculated relative to DMSO control and IC₅₀ values were obtained from dose-response data fitted to a 4-parameter curve.

### Example 33

### Biological Activity of Selected Compounds

Results of testing selected compounds in the Src Biochemical Assay Protocol, Cell Viability Protocol, and Cellular Proliferation Protocol described in Examples 30-32, above, are presented in Table 19. Compounds denoted as “A” had an IC₅₀ less than or equal to 0.01 μM, compounds denoted as “B” had an IC₅₀ between 0.01 μM and 1.0 μM, compounds denoted as “C” had an IC₅₀ between 1 μM and 10 μM, and compounds denoted as “D” had an IC₅₀ greater than 10 μM. Compounds denoted as “---” were not tested.

TABLE 19
Results of Src Biochemical Assay Protocol,
Cell Viability Protocol, and Cellular Proliferation Protocol

| | | | Cellular |
|---|---|---|---|
| | Src Biochemical | Cell Viability | Proliferation |
| Compound | Assay Protocol | Protocol | Protocol |
| I-1 | B | B | A |
| I-2 | B | — | — |
| I-3 | A | — | — |
| I-4 | B | B | A |
| I-5 | A | A | A |
| I-6 | A | B | — |
| I-7 | B | B | A |
| I-8 | B | B | — |
| I-9 | B | — | — |
| I-10 | B | — | — |
| I-11 | B | — | — |
| I-12 | B | — | — |
| I-13 | C | — | — |
| I-14 | B | — | — |
| I-15 | B | — | — |
| I-16 | B | — | — |
| I-17 | A | — | — |
| I-18 | B | — | — |
| I-19 | A | A | A |
| I-20 | B | B | A |
| I-21 | B | — | — |
| I-22 | B | — | — |
| I-23 | B | B | B |
| I-24 | B | — | — |
| I-25 | A | A | A |
| I-26 | B | — | — |
| I-27 | B | B | A |
| I-28 | B | — | — |
| I-29 | B | B | — |
| I-30 | B | — | — |
| I-31 | B | — | — |
| I-32 | B | B | A |
| I-33 | B | B | B |
| I-34 | C | — | — |
| I-35 | B | B | — |
| I-36 | B | — | — |
| I-37 | A | — | — |
| I-38 | B | — | — |
| I-39 | B | — | — |
| I-40 | B | C | C |
| I-41 | B | B | — |
| I-42 | B | B | B |
| I-43 | A | — | — |
| I-44 | A | A | A |
| I-45 | A | A | A |
| I-46 | B | — | — |
| I-47 | B | — | — |
| I-48 | B | B | — |
| I-49 | A | A | A |
| I-50 | B | B | B |
| I-51 | B | B | A |
| I-52 | B | B | B |
| I-53 | A | — | — |
| I-54 | A | B | A |
| I-55 | B | B | — |
| I-56 | B | B | B |
| I-57 | B | B | B |
| I-58 | B | B | — |
| I-59 | B | B | — |
| I-60 | B | B | — |
| I-61 | B | C | C |
| I-62 | B | B | — |
| I-63 | B | B | — |
| I-64 | B | C | — |
| I-65 | B | B | — |
| I-66 | B | B | — |
| I-67 | B | B | — |
| I-68 | B | B | — |
| I-69 | B | B | — |
| I-70 | A | A | — |
| I-71 | B | A | — |
| II-1 | B | A | A |
| II-2 | B | B | B |
| II-3 | B | B | B |
| II-4 | B | B | B |
| II-5 | B | B | B |
| II-6 | B | B | B |
| II-7 | B | B | B |
| II-8 | B | B | B |
| II-9 | C | B | B |
| II-10 | C | B | C |
| II-11 | C | B | B |
| II-12 | B | B | B |
| II-13 | B | B | B |
| II-14 | C | C | C |
| II-15 | C | B | B |
| II-16 | C | B | B |
| II-17 | B | A | A |
| II-18 | B | B | B |
| II-19 | B | C | B |
| II-20 | B | B | B |
| II-21 | B | B | B |
| II-22 | B | B | B |
| II-23 | B | A | A |
| II-24 | A | A | A |
| II-25 | B | B | B |
| II-26 | B | B | B |
| II-27 | A | A | A |
| II-28 | B | B | B |
| II-29 | C | C | C |
| II-30 | C | B | B |
| II-31 | C | B | B |
| II-32 | B | B | B |
| II-33 | B | B | B |
| II-34 | B | B | B |
| II-35 | B | B | B |
| II-36 | B | B | B |
| II-37 | B | B | B |
| II-38 | B | B | B |
| II-39 | A | A | A |
| II-40 | A | A | A |
| II-41 | A | A | A |
| II-42 | B | B | B |
| II-43 | B | A | — |
| II-44 | B | A | — |
| II-45 | B | B | B |
| II-46 | B | B | — |
| II-47 | B | B | — |
| II-48 | B | A | A |
| II-49 | B | B | B |
| II-50 | B | C | C |
| II-51 | B | B | B |
| II-52 | B | C | B |
| II-53 | A | B | B |
| II-54 | B | B | B |
| II-55 | A | B | A |
| II-56 | B | B | B |
| II-57 | B | C | C |
| II-58 | B | B | B |
| II-59 | B | C | B |
| II-60 | B | B | B |
| II-61 | B | C | B |
| II-62 | A | B | B |
| II-63 | C | C | C |
| II-64 | B | B | B |
| II-65 | A | B | B |
| II-66 | B | B | B |
| II-67 | B | B | B |
| II-68 | C | C | B |
| II-69 | B | B | B |
| II-70 | C | B | B |
| II-71 | B | B | B |
| II-72 | B | B | B |
| II-73 | B | B | B |
| II-74 | B | B | B |
| II-75 | C | C | C |
| II-76 | B | C | C |
| II-77 | C | C | C |
| II-78 | C | — | — |
| II-79 | B | B | B |
| II-80 | C | B | C |
| II-81 | B | B | B |
| II-82 | A | A | B |
| II-83 | A | B | B |
| II-84 | A | B | B |
| II-85 | B | B | B |
| II-86 | A | B | A |
| II-87 | B | B | B |
| II-88 | C | C | C |
| II-89 | B | B | B |
| II-90 | B | B | B |
| II-91 | B | B | B |
| II-92 | B | B | B |
| II-93 | C | C | C |
| II-94 | B | B | B |
| II-95 | A | B | B |
| II-96 | B | B | B |
| II-97 | B | B | B |
| II-98 | B | B | B |
| II-99 | B | B | B |
| II-100 | C | B | B |
| II-101 | B | B | B |
| II-102 | B | B | B |
| II-103 | B | B | B |
| II-104 | B | B | B |
| II-105 | B | B | B |
| II-106 | B | B | B |
| II-107 | C | C | C |
| II-108 | A | A | A |
| II-109 | A | B | B |
| II-110 | A | B | A |
| II-111 | A | B | B |
| II-112 | A | B | B |
| II-113 | A | B | B |
| II-114 | A | B | B |
| II-115 | A | B | B |
| II-116 | A | A | A |
| II-117 | A | A | A |
| II-118 | A | B | B |
| II-119 | A | B | B |
| II-120 | B | B | B |
| II-121 | B | — | — |
| II-125 | B | — | — |
| II-126 | A | B | B |
| II-127 | A | B | B |
| II-128 | C | B | C |
| II-129 | C | B | C |
| II-130 | A | B | B |
| II-131 | A | A | A |
| II-132 | A | A | A |
| II-133 | A | B | B |
| II-134 | A | B | B |
| II-135 | B | B | B |
| II-137 | A | C | — |
| II-138 | A | B | — |
| II-139 | B | B | — |
| II-140 | B | B | — |
| II-141 | A | — | — |
| II-142 | A | B | — |
| II-143 | B | C | — |
| II-144 | B | C | — |
| II-145 | B | C | — |
| II-146 | A | B | — |
| II-147 | A | B | — |
| II-148 | B | — | — |
| II-149 | A | — | — |
| II-150 | B | — | — |
| II-151 | A | — | — |
| II-152 | A | — | — |
| II-153 | A | — | — |
| II-154 | B | — | — |
| II-155 | A | — | — |
| II-156 | B | — | — |
| II-157 | A | — | — |
| II-158 | B | — | — |
| II-159 | B | — | — |
| II-160 | A | B | B |
| II-161 | A | B | B |
| II-162 | B | B | — |
| II-163 | A | B | B |
| II-164 | B | A | A |
| II-165 | B | A | A |
| II-166 | A | A | A |
| II-167 | B | A | B |
| II-168 | B | A | A |
| II-169 | B | A | A |
| II-170 | B | A | A |
| II-171 | B | A | A |
| II-172 | B | B | B |
| II-173 | A | A | B |
| II-174 | A | B | B |
| II-175 | B | A | B |
| II-176 | B | A | A |
| II-177 | B | B | B |
| II-178 | B | B | B |
| II-179 | B | B | B |
| II-180 | B | C | C |
| II-181 | B | C | C |
| II-182 | A | A | A |
| II-183 | B | B | C |
| II-184 | B | C | C |
| II-185 | B | C | C |
| II-186 | A | A | A |
| II-187 | B | A | A |
| II-188 | B | B | B |
| II-189 | B | C | C |
| II-190 | B | A | B |
| II-191 | B | B | B |
| II-192 | B | C | C |
| II-193 | B | C | C |
| II-194 | B | B | B |
| II-195 | B | B | B |
| II-196 | B | A | A |
| II-197 | B | B | B |
| II-198 | A | A | A |
| II-199 | A | A | A |
| II-200 | B | B | B |
| II-201 | B | B | B |
| II-202 | B | B | B |
| II-203 | B | B | B |
| II-204 | B | B | B |
| II-205 | B | B | B |
| II-206 | B | A | A |
| II-207 | B | B | B |
| II-208 | B | B | B |
| II-209 | A | A | A |
| II-210 | A | A | A |
| II-211 | A | A | A |
| II-212 | A | A | A |
| II-213 | C | C | C |
| II-214 | A | A | A |
| II-215 | B | B | B |
| II-216 | B | B | B |
| II-217 | B | B | B |
| II-218 | B | B | B |
| II-219 | A | A | A |
| II-220 | B | B | B |
| II-221 | A | A | A |
| II-222 | B | B | A |
| II-223 | B | B | B |
| II-224 | B | B | — |
| II-225 | B | B | — |
| II-226 | B | B | B |
| II-227 | B | B | B |
| II-228 | B | B | B |
| II-229 | A | A | A |
| II-230 | B | B | B |
| II-231 | B | B | — |
| II-232 | B | B | B |
| II-233 | B | B | — |
| II-234 | B | B | B |
| II-235 | B | C | — |
| II-236 | B | B | — |
| II-237 | B | B | B |
| II-238 | B | B | B |
| II-239 | B | B | B |
| II-240 | B | B | B |
| II-241 | B | B | B |
| II-242 | B | B | — |
| II-243 | B | B | B |
| II-244 | B | B | — |
| II-245 | B | B | — |
| II-246 | B | A | B |
| II-247 | A | A | A |
| II-248 | A | B | B |
| II-249 | B | A | A |
| II-250 | B | A | B |
| II-251 | B | B | B |
| II-252 | B | B | B |
| II-253 | B | B | B |
| II-254 | B | B | B |
| II-255 | B | B | B |
| II-256 | B | B | B |
| II-257 | A | B | B |
| II-258 | B | B | B |
| II-259 | B | B | B |
| II-260 | B | B | B |
| II-261 | B | C | B |
| II-262 | D | C | C |
| II-263 | C | C | C |
| II-264 | C | C | C |
| II-265 | B | B | — |
| II-266 | B | C | C |
| II-267 | B | C | — |
| II-268 | B | C | C |
| II-269 | B | B | B |
| II-271 | A | A | A |
| II-272 | A | A | A |
| II-273 | A | B | B |
| II-274 | B | C | C |
| II-275 | B | B | B |
| II-276 | B | B | B |
| II-277 | A | A | A |
| II-278 | A | B | B |
| II-279 | B | B | B |
| II-280 | A | A | B |
| II-281 | B | B | B |
| II-282 | A | B | B |
| II-283 | A | B | B |
| II-284 | A | B | B |
| II-285 | A | B | A |
| II-286 | A | B | A |
| II-287 | A | B | B |
| II-288 | A | A | A |
| II-289 | A | B | B |
| II-290 | A | B | B |
| II-291 | A | B | B |
| II-292 | B | B | B |
| II-293 | A | A | A |
| II-294 | A | A | A |
| II-295 | A | B | B |
| II-296 | A | B | A |
| II-297 | B | B | B |
| II-298 | B | B | B |
| II-299 | B | B | B |
| II-300 | A | B | B |
| II-301 | C | B | C |
| II-302 | B | — | — |
| II-303 | B | B | B |
| II-304 | B | B | B |
| II-306 | B | — | — |
| II-307 | B | B | B |
| II-309 | B | B | B |
| II-310 | A | A | A |
| II-311 | A | — | — |
| II-313 | B | B | B |
| II-314 | D | B | B |
| II-315 | B | A | B |
| II-316 | B | A | A |
| II-317 | B | A | B |
| II-318 | B | B | B |
| II-319 | B | B | B |
| II-320 | B | B | B |
| II-321 | B | B | B |
| II-322 | A | B | B |
| II-323 | A | A | B |
| II-324 | B | B | B |
| II-325 | B | C | C |
| II-326 | B | B | B |
| II-327 | A | B | B |
| II-328 | B | B | B |
| II-329 | B | B | B |
| II-330 | A | A | A |
| III-1 | B | A | A |
| III-2 | B | A | A |
| III-3 | A | B | B |
| III-4 | B | — | — |
| III-5 | C | C | — |
| III-6 | B | — | — |
| III-7 | B | — | — |
| III-8 | B | — | — |
| III-9 | B | — | — |
| III-10 | B | — | — |
| III-11 | B | — | — |
| III-12 | A | — | — |
| III-13 | A | — | — |
| III-14 | B | — | — |
| III-15 | B | — | — |
| III-16 | B | — | — |
| III-18 | B | — | — |
| III-19 | B | — | — |
| III-20 | B | — | — |
| III-21 | B | — | — |
| III-22 | B | — | — |
| III-23 | B | — | — |
| III-24 | A | — | — |
| III-25 | B | — | — |
| III-26 | B | — | — |
| III-27 | B | — | — |
| III-29 | B | — | — |
| III-31 | B | — | — |
| III-32 | B | — | — |
| III-33 | B | — | — |
| III-35 | B | — | — |
| III-36 | B | — | — |
| III-37 | B | — | — |
| III-38 | B | — | — |
| III-39 | B | — | — |
| III-40 | B | — | — |
| III-41 | B | — | — |
| III-43 | B | — | — |
| III-45 | A | — | — |
| III-46 | B | — | — |
| III-47 | B | — | — |
| III-48 | B | — | — |
| III-49 | B | — | — |
| III-50 | B | — | — |
| III-51 | B | — | — |
| III-52 | B | — | — |
| III-53 | A | — | — |
| III-54 | C | — | — |
| III-55 | A | — | — |
| III-56 | B | — | — |
| III-57 | B | — | — |
| III-58 | B | — | — |
| III-59 | B | — | — |
| III-60 | B | — | — |
| III-61 | B | — | — |
| III-62 | B | — | — |
| III-63 | A | — | — |
| III-64 | B | — | — |
| III-65 | B | — | — |
| III-66 | B | — | — |
| III-67 | B | — | — |
| III-68 | B | — | — |
| III-69 | B | — | — |
| III-70 | A | — | — |
| III-71 | B | — | — |
| III-73 | B | — | — |
| III-74 | D | — | — |
| III-75 | B | — | — |
| III-76 | A | — | — |
| III-77 | B | — | — |
| III-78 | B | — | — |
| III-79 | B | — | — |
| III-80 | B | — | — |
| III-81 | B | — | — |
| III-82 | B | — | — |
| III-83 | B | — | — |
| III-84 | B | — | — |
| III-85 | A | — | — |
| III-86 | B | — | — |
| III-87 | B | — | — |
| III-88 | A | — | — |
| III-89 | B | — | — |
| III-90 | B | — | — |
| III-91 | B | — | — |
| III-92 | B | — | — |
| III-93 | C | — | — |
| III-94 | C | — | — |
| III-95 | C | — | — |

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound of Formula (V) or (VI), or a pharmaceutically acceptable salt thereof: 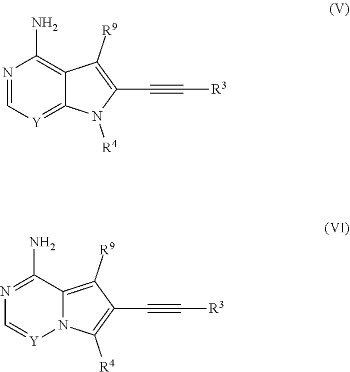
wherein:
Y is CR^{Y} or N;
R³ is —Z¹-L¹-Z²-L²-R⁶;
Z¹ is C₁₋₆ aliphatic; a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z¹ is substituted by m instances of R⁷, and n instances of R⁸;
Z² is a covalent bond; C₁₋₆ aliphatic; a 3-7 membered monocyclic heterocyclic ring having 1-2 heteroatoms independently selected from nitrogen, oxygen, and sulfur; or a 6-10 membered bicyclic heterocyclic ring having 1-2 heteroatoms independently selected from nitrogen, oxygen, and sulfur; wherein Z² is substituted by m instances of R⁷, and n instances of R⁸;
each of L¹ and L² is independently a covalent bond, —N(H)—, —N(R⁵)—, or —C(O)—;
R⁴ is hydrogen, or an optionally substituted group selected from the group consisting of C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R⁶ is —CN, 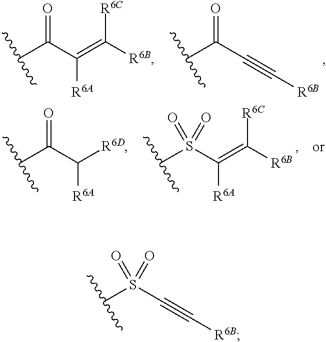
each of R^{6A}, R^{6B}, and R^{6C} is independently hydrogen, halogen, —CN, —C(O)R, —C(O)OR, —C(O)NR₂, —C(O)N(R)OR, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R^{6D} is halogen or —OS(O)₂R;
each instance of R⁵, R⁷, R⁸, and R¹⁰ is independently hydrogen or R^{L};
each instance of R^{L} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R^{Y} is hydrogen or R^{L};
R⁹ is —R¹—X—R²; or a ring selected from the group consisting of phenyl; a 5-6 membered monocyclic heteroaryl ring having 1-4 heteroatoms independently selected from nitrogen, oxygen, and sulfur; and an 8-10 membered bicyclic ring having 1-4 heteroatoms independently selected from nitrogen, oxygen, and sulfur; wherein said ring is substituted by p instances of R¹⁰;
R¹ is selected from arylene and heteroarylene; each of which is optionally substituted;
R² is selected from aryl, heteroaryl, and cycloalkyl; each of which is optionally substituted;
X is a C₁₋₂ bivalent hydrocarbon chain wherein one or more methylene units of the chain are optionally and independently replaced by —CH(R^{L})—, C₃₋₅ cycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each instance of m and n is independently 0-4; and
p is 0-4.

2. The compound of claim 1, wherein at least one of Z¹ and Z² is a cyclic group.

3. The compound of claim 1, wherein R³ is:

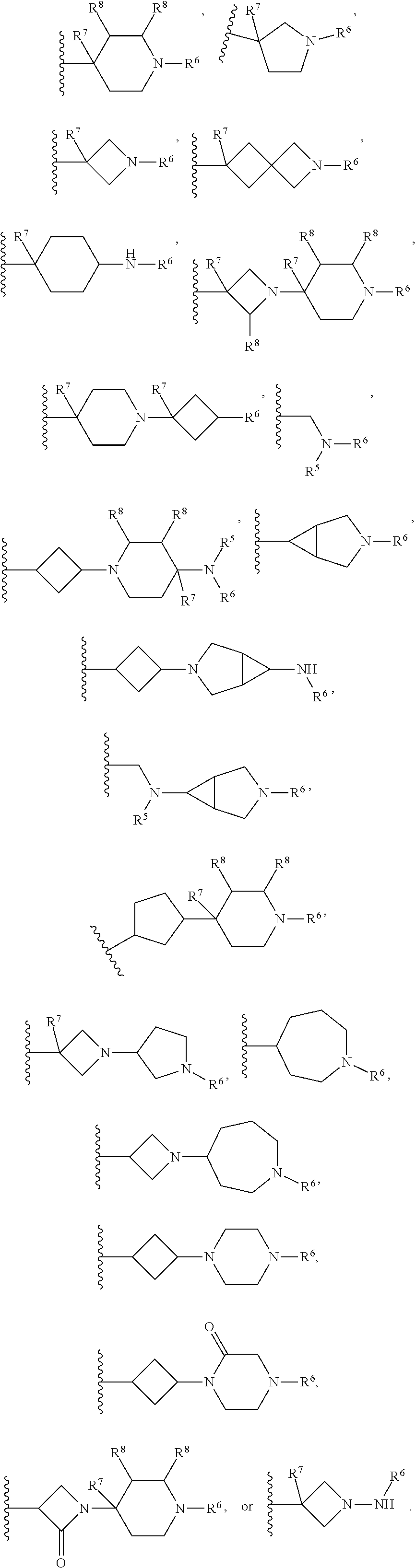

4. The compound of claim 1, wherein each instance of R⁷ is independently hydrogen, —OH, —CH₃, —F, or —OMe.

5. The compound of claim 1, wherein each instance of R⁸ is independently hydrogen, —OH, —CH₃, —F, —CH₂OH, —CH₂OMe, or —OMe.

6. The compound of claim 1, wherein R⁴ is hydrogen or C₁₋₆ aliphatic optionally substituted with —OH, —O—(C₁₋₆ aliphatic), —C(O)NH₂, —C(O)OH, or —C(O)—O—(C₁₋₆ aliphatic).

7. The compound of claim 1, wherein R⁴ is C₁₋₃ aliphatic.

8. The compound of claim 1, wherein R⁹ is —R¹—X—R².

9. The compound of claim 1, wherein R¹ is phenylene or pyridinylene, each of which is optionally substituted with fluoro, chloro, —CN, —OMe, —OEt, —OCF₃, —OCHF₂, —OCH₂F, or —CH₃.

10. The compound of claim 1, wherein R² is phenyl or pyridinyl, each of which is optionally substituted with —F, —Cl, —CN, or —CH₃.

11. The compound of claim 1, wherein X is —O—, —N(H)—, —OCH₂—, or —C(H)(CN)—.

12. The compound of claim 1, wherein R⁹ is an 8-10 membered bicyclic ring having 1-4 heteroatoms independently selected from nitrogen, oxygen, and sulfur; wherein said ring is substituted by p instances of R¹⁰.

13. The compound of claim 1, wherein R⁹ is 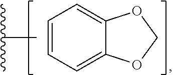
which is substituted by p instances of R¹⁰.

14. The compound of claim 1, wherein R⁶ is CN, —C(O)C(H)CH₂, —C(O)CH₂Cl, —C(O)C(H)(Cl)CH₃, —S(O)₂C(H)CH₂, —S(O)₂C(H)CH—CH₃, —C(O)CCMe, —C(O)C(CH₃)CH₂, or —C(O)C(H)C(H)CH₃.

15. The compound of claim 1, wherein R⁶ is —C(O)C(H)CH₂.

16. The compound of claim 1, wherein Y is N.

17. The compound of claim 1, wherein the compound is selected from those depicted in Tables 3-19, or a pharmaceutically acceptable salt thereof.

18. (canceled)

19. A pharmaceutical composition comprising the compound of claim 1, or a pharmaceutically acceptable salt thereof, and a pharmaceutically acceptable carrier.

20. A method for treating a Src-mediated disorder in a subject, comprising administering to the subject a therapeutically effective amount of the compound of claim 1, or a pharmaceutically acceptable salt thereof.

21. A method for treating a proliferative disorder or cancer in a subject, comprising administering to the subject a therapeutically effective amount of the compound of claim 1, or a pharmaceutically acceptable salt thereof.

22. (canceled)
